(19) United States
(12) Patent Application Publication
(10) Pub. No.: US 2025/0144036 A1
(43) Pub. Date: May 8, 2025

(54) COMPOUNDS AND COMPOSITIONS FOR DELIVERY OF THERAPEUTIC AGENTS

(71) Applicant: ModernaTX, Inc., Cambridge, MA (US)

(72) Inventors: Kerry E. BENENATO, Cambridge, MA (US); Juneyoung LEE, Cambridge, MA (US); Mohindra SEEPERSAUD, Cambridge, MA (US)

(21) Appl. No.: 18/716,220

(22) PCT Filed: Dec. 9, 2022
(86) PCT No.: PCT/US2022/052347

§ 371 Date: Jun. 4, 2024

Related U.S. Application Data
(60) Provisional application No. 63/288,317, filed on Dec. 10, 2021.

(51) Int. Cl. A61K 9/51; C07C 229/06

(52) U.S. Cl. CPC A61K 9/5123; C07C 229/06

(57) ABSTRACT

The disclosure features novel lipids and compositions involving the same. Lipid nanoparticles (e.g., empty LNPs or loaded LNPs) include a novel cationic lipid as well as additional lipids such as ionizable lipids, phospholipids, structural lipids, and PEG lipids. Lipid nanoparticles (e.g., empty LNPs or loaded LNPs) further including therapeutic and/or prophylactics such as RNA are useful in the delivery of therapeutic and/or prophylactics to mammalian cells or organs to, for example, regulate polypeptide, protein, or gene expression.

### RELATED APPLICATION

This application claims priority to, and the benefit of, U.S. Application No. 63/288,317, filed Dec. 10, 2021, the entire content of which is incorporated herein by reference.

### FIELD OF DISCLOSURE

The present disclosure provides novel cationic lipids, compositions comprising such lipids, and methods involving lipid nanoparticle compositions to deliver one or more therapeutic and/or prophylactics to and/or produce polypeptides in mammalian cells or organs. In addition to a novel cationic lipid, lipid nanoparticle compositions of the disclosure may include one or more ionizable amino lipids, phospholipids including polyunsaturated lipids, PEG lipids, structural lipids, and/or therapeutic and/or prophylactics in specific fractions.

### BACKGROUND

The effective targeted delivery of biologically active substances such as small molecule drugs, proteins, and nucleic acids represents a continuing medical challenge. In particular, the delivery of nucleic acids to cells is made difficult by the relative instability and low cell permeability of such species. Thus, there exists a need to develop methods and compositions to facilitate the delivery of therapeutic and/or prophylactics such as nucleic acids to cells.

Lipid-containing nanoparticle compositions, liposomes, and lipoplexes have proven effective as transport vehicles into cells and/or intracellular compartments for biologically active substances such as small molecule drugs, proteins, and nucleic acids. Such compositions generally include one or more “cationic” and/or amino (ionizable) lipids, phospholipids including polyunsaturated lipids, structural lipids (e.g., sterols), and/or lipids containing polyethylene glycol (PEG lipids). Cationic and/or ionizable lipids include, for example, amine-containing lipids that can be readily protonated. Though a variety of such lipid-containing nanoparticle compositions have been demonstrated, improvements in safety, efficacy, and specificity are still lacking.

### SUMMARY

The present disclosure provides novel cationic lipids and compositions (e.g. lipid nanoparticles) and methods involving the same.

In some aspects, the disclosure relates to a cationic lipid of Formula (I):

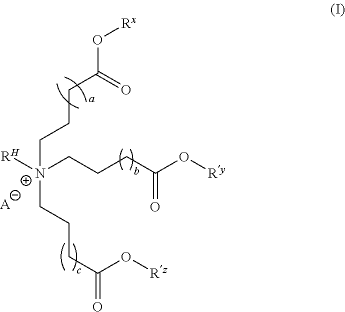

or an isomer thereof, wherein:
R′^{x} is:

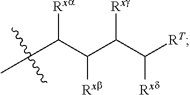

wherein R′^{y} is:

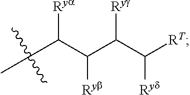

and R′^{z} is:

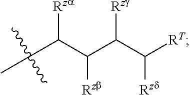

wherein

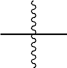

denotes a point of attachment;
R^{xα}, R^{xγ}, R^{xγ}, and R^{xδ} are each independently selected from the group consisting of H, C₁₋₁₂ alkyl, and C₂₋₁₂ alkenyl;
R^{yα}, R^{yβ}, R^{yγ}, and R^{yδ} are each independently selected from the group consisting of H, C₁₋₁₂ alkyl, and C₂₋₁₂ alkenyl;
R^{zα}, R^{zβ}, R^{zγ}, and R^{zδ} are each independently selected from the group consisting of H, C₁₋₁₂ alkyl, and C₂₋₁₂ alkenyl;
R^{H} is —(CH₂)_{q}OH, wherein q is selected from 1, 2, 3, 4, and 5; each R^{T} is independently selected from C₁₋₁₂ alkyl and C₂₋₁₂ alkenyl;
a is selected from 1, 2, 3, 4, 5, 6, 7, 8, and 9;
b is selected from 1, 2, 3, 4, 5, 6, 7, 8, and 9;
c is selected from 1, 2, 3, 4, 5, 6, 7, 8, and 9; and
A⁻ is any pharmaceutically acceptable anion.

In some embodiments, the cationic lipid of Formula (I) has one of the following structures:

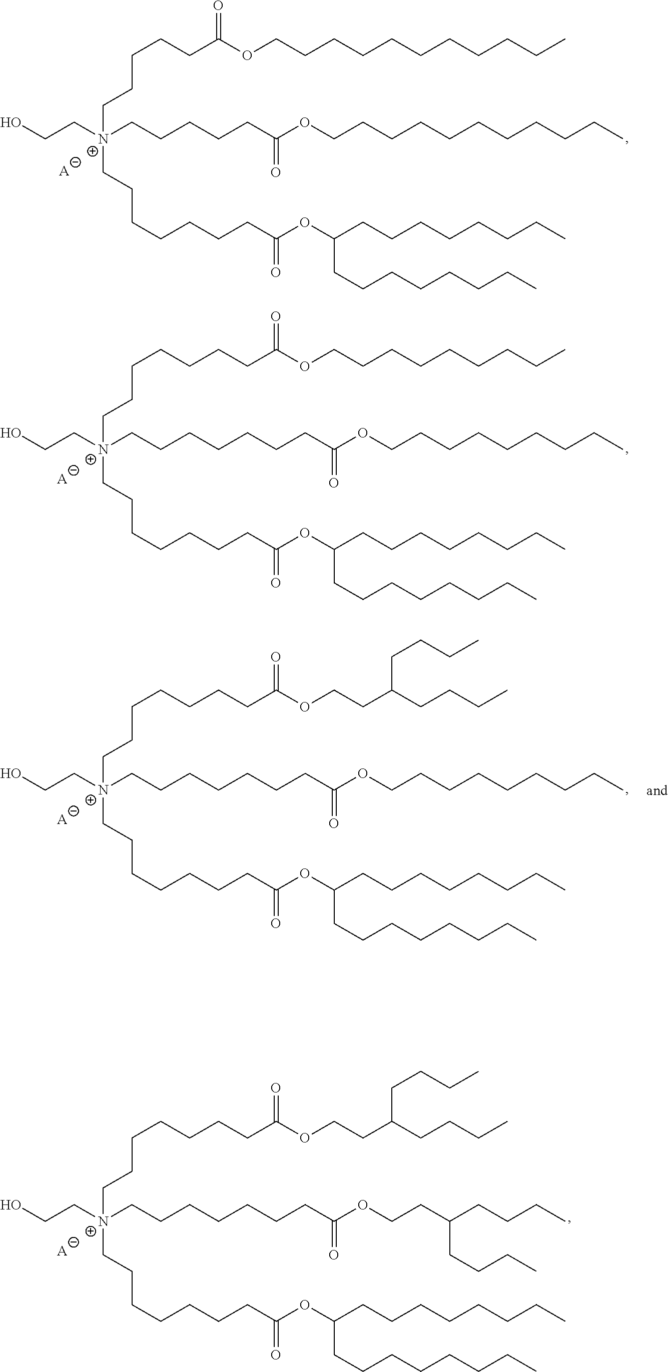

wherein A⁻ is selected from chloride, bromide, iodide, hydroxide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate.

In some embodiments, the cationic lipid of Formula (I) has one of the following structures:

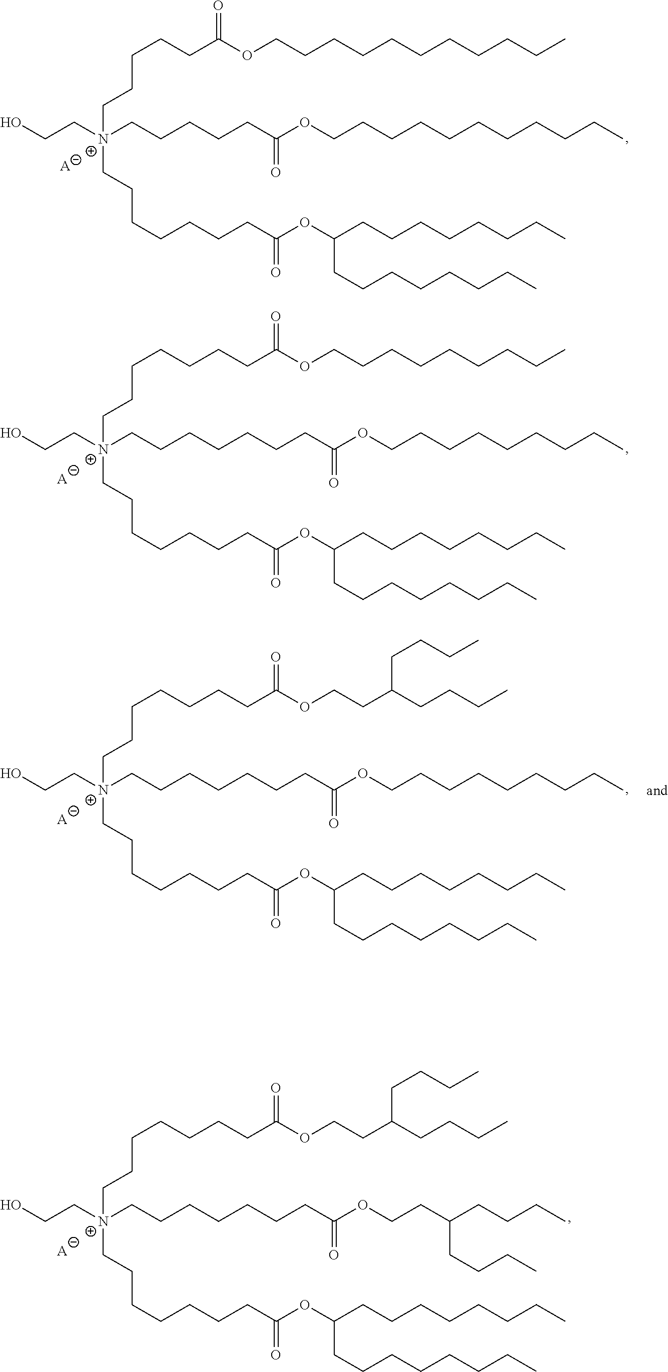

wherein A⁻ is bromide, chloride, hydroxide, or a combination thereof. In some embodiments, A⁻ is bromide or hydroxide. In some embodiments A⁻ is chloride or hydroxide. In some embodiments A⁻ is bromide. In some embodiments A⁻ is chloride. In some embodiments A⁻ is hydroxide.

### DETAILED DESCRIPTION

The present disclosure provides novel cationic lipids including a central amine moiety and at least one biodegradable group. The cationic lipids described herein may be advantageously used in lipid nanoparticles (e.g., empty LNPs or loaded LNPs) for the delivery of therapeutic and/or prophylactics to mammalian cells or organs. For example, the cationic lipids described herein may be advantageously used in lipid nanoparticles (e.g., empty LNPs or loaded LNPs) for the delivery of therapeutic and/or prophylactics to specific mammalian cells or organs. In some embodiments, the cationic lipids described herein may be advantageously used in lipid nanoparticles (e.g., empty LNPs or loaded LNPs) for the delivery of therapeutic and/or prophylactics to endothelial cells or the lung.

The present disclosure also provides lipid nanoparticles (LNPs) comprising the novel cationic lipids for delivery of a therapeutic and/or prophylactic agent to endothelial cells. For example, such LNPs can be used to deliver therapeutic and/or prophylactic agents, e.g., mRNA therapeutics, to endothelial cells. In some embodiments, such LNPs can be used to deliver nucleic acid molecules for gene editing, small molecules, or other payloads to ameliorate endothelial cell dysfunction. In some embodiments, such LNPs can be used to deliver antigens to endothelial cells, which are major participants in and regulators of inflammatory reactions. Resting endothelial cells prevent coagulation, control blood flow and passage of proteins from blood into tissues, and inhibit inflammation. In some embodiments, the antigen is in the form of an mRNA construct present in the LNP resulting in the expression of a polypeptide or peptide such that an immune response to the antigen is produced.

LNPs are an ideal platform for the safe and effective delivery of therapeutic and/or prophylactic agents, e.g., mRNAs, to target cells. LNPs have the unique ability to deliver therapeutic and/or prophylactic agents (e.g., nucleic acids, e.g. mRNAs) by a mechanism involving cellular uptake, intracellular transport

and endosomal release or endosomal escape. Some embodiments provided herein feature LNPs that have improved properties. In some embodiments, the LNPs provided herein comprise a lipid nanoparticle core, a therapeutic and/or prophylactic agent encapsulated within the core for delivery into a cell, and a cationic agent disposed primarily on the outer surface of the nanoparticle. Without being bound by a particular theory, LNPs having a cationic agent disposed primarily on the outer surface of the core can improve accumulation of the LNP in cells such as human pulmonary endothelial cells and also improve function of the therapeutic and/or prophylactic agent, e.g., as measured by mRNA expression in cells, e.g., endothelial cells in the lungs.

In some embodiments, provided herein is a loaded LNP comprising:
(a) a lipid nanoparticle core,
(b) a therapeutic and/or prophylactic agent encapsulated within the core for delivery into a cell, and
(c) a cationic agent (e.g., a cationic lipid of Formula (I)) disposed primarily on the outer surface of the core, wherein the loaded LNP has a greater than neutral zeta potential at physiologic pH.

In some embodiments, provided herein is a loaded LNP comprising:
(a) a loaded LNP core comprising:
   (i) an ionizable lipid,
   (ii) a phospholipid,
   (iii) a structural lipid, and
   (iv) a PEG-lipid, and
(b) a therapeutic and/or prophylactic agent encapsulated within the core for delivery into a cell, and
(c) a cationic agent (e.g., a cationic lipid of Formula (I)).

In some embodiments, provided herein is a loaded LNP comprising:
(a) a lipid nanoparticle core comprising:
   (i) an ionizable lipid,
   (ii) a phospholipid,
   (iii) a structural lipid, and
   (iv) a PEG-lipid, and
(b) a polynucleotide or polypeptide payload therapeutic and/or prophylactic agent encapsulated within the core for delivery into a cell, and
(c) a cationic agent (e.g., a cationic lipid of Formula (I)) disposed primarily on the outer surface of the core.

In some embodiments, provided herein is a loaded LNP comprising:
(a) a lipid nanoparticle core,
(b) a therapeutic and/or prophylactic agent encapsulated within the core for delivery into a cell, and
(c) a cationic agent (e.g., a cationic lipid of Formula (I)),
   wherein the loaded LNP exhibits a cellular accumulation of at least about 20% of cells and exhibits about 5% or greater expression in cells in a population of cells to which the loaded LNP is administered. In some embodiments, the loaded LNP exhibits a cellular accumulation in about 1% to about 75%, 5% to about 50%, about 10% to about 40%, or about 15% to about 25% of cells in a population of cells to which the loaded LNP is administered.

In some embodiments, the loaded LNP exhibits expression in from about 0.5% to about 50%, from about 1% to about 40%, from about 3% to about 20%, or from about 5% to about 15% of cells in which the loaded LNP is accumulated.

In some embodiments aspect, provided herein is a loaded LNP comprising:
(a) a lipid nanoparticle core,
(b) a therapeutic and/or prophylactic agent encapsulated within the core for delivery into a cell, and
(c) a cationic agent (e.g., a cationic lipid of Formula (I)) disposed primarily on the outer surface of the core,
   wherein the loaded LNP exhibits a cellular accumulation in at least about 20% of cells in a population of cells to which the loaded LNP is administered and exhibits about 5% or greater expression in cells in which the loaded LNP is accumulated. In some embodiments, the loaded LNP exhibits a cellular accumulation in from about 1% to about 75%, from about 5% to about 50%, from about 10% to about 40%, or from about 15% to about 25% of cells in a population of cells to which the loaded LNP is administered. In some embodiments, the loaded LNP exhibits from about 0.5% to about 50%, from about 1% to about 40%, from about 3% to about 20%, or from about 5% to about 15% expression in cells in which the loaded LNP is accumulated.

In some embodiments, provided herein is a loaded LNP comprising:
(a) a lipid nanoparticle core,
(b) a therapeutic and/or prophylactic agent encapsulated within the core for delivery into a cell, and
(c) a cationic agent (e.g., a cationic lipid of Formula (I)) disposed primarily on the outer surface of the core,
   wherein the therapeutic and/or prophylactic agent expresses a protein and wherein loaded LNP exhibits protein expression of from about 0.5% to 50% in cells in a population of cells to which the loaded LNP is administered. In some embodiments, the loaded LNP exhibits protein expression of from about 0.1% to about 60%, from about 0.5% to about 40%, from about 1% to about 30%, or from about 1% to about 20% in cells in which the loaded LNP is accumulated.

In some embodiments, provided herein is a loaded LNP comprising:
(a) a lipid nanoparticle core,
(b) a therapeutic and/or prophylactic agent encapsulated within the core for delivery into a cell, and
(c) a cationic agent (e.g., a cationic lipid of Formula (I)),
   wherein the loaded LNP exhibits protein expression of about 0.5% to 50% in cells in which the loaded LNP is accumulated. In some embodiments, the loaded LNP exhibits protein expression of from about 0.1% to about 60%, from about 0.5% to about 40%, from about 1% to about 30%, or from about 1% to about 20% in cells in which the loaded LNP is accumulated.

In some embodiments, provided herein is a loaded LNP comprising:
(a) a lipid nanoparticle core,
(b) a therapeutic and/or prophylactic agent encapsulated within the core for delivery into a cell, and
(c) a cationic agent (e.g., a cationic lipid of Formula (I)),
   wherein the loaded LNP exhibits a cellular accumulation of at least about 20% in endothelial cells and exhibits about 5% or greater expression in endothelial cells in which the loaded LNP is accumulated. In some embodiments, the loaded LNP exhibits a cellular accumulation of from about 1% to about 75%, from about 5% to about 50%, from about 10% to about 40%, or from about 15% to about 25% of endothelial cells in a population of endothelial cells cells to which the loaded LNP is administered. In some embodiments, the loaded LNP exhibits from about 0.5% to about 50%, from about 1% to about 40%, from about 3% to about 20%, or from about 5% to about 15% expression in endothelial cells in which the loaded LNP is accumulated.

In some embodiments, provided herein is a loaded LNP comprising:
(a) a lipid nanoparticle core,
(b) a therapeutic and/or prophylactic agent encapsulated within the core for delivery into a cell, and
(c) a cationic agent (e.g., a cationic lipid of Formula (I)),
   wherein the loaded LNP exhibits protein expression of about 0.5% to 50% of endothelial cells in which the loaded LNP is accumulated. In some embodiments, the loaded LNP exhibits protein expression of from about 0.1% to about 60%, from about 0.5% to about 40%, about 1% to about 30%, or from about 1% to about 20% of endothelial cells in which the loaded LNP is accumulated.

In some embodiments, provided herein is a loaded LNP comprising:
(a) a lipid nanoparticle core,
(b) a therapeutic and/or prophylactic agent encapsulated within the core for delivery into a cell, and
(c) a cationic agent (e.g., a cationic lipid of Formula (I)),
   wherein the loaded LNP exhibits protein expression in about 0.5% to about 50% of lung cells in which the loaded LNP is accumulated. In some embodiments, the loaded LNP exhibits protein expression of from about 0.1% to about 60%, from about 0.5% to about 40%, from about 1% to about 30%, or from about 1% to about 20% of pulmonary endothelial cells in which the loaded LNP is accumulated.

In some embodiments, provided herein is a loaded LNP comprising:
(a) a lipid loaded LNP core,
(b) a therapeutic and/or prophylactic agent encapsulated within the core for delivery into a cell, and
(c) a cationic agent (e.g., a cationic lipid of Formula (I)), wherein the loaded LNP exhibits a cellular accumulation of at least about 20% in respiratory endothelial cells and exhibits about 5% or greater expression in respiratory endothelial cells in which the loaded LNP is accumulated. In some embodiments, the loaded LNP exhibits a cellular accumulation of from about 1% to about 75%, from about 5% to about 50%, from about 10% to about 40%, or from about 15% to about 25% of respiratory endothelial cells in a population of respiratory endothelial cells to which the loaded LNP is administered. In some embodiments, the loaded LNP exhibits from about 0.5% to about 50%, from about 1% to about 40%, from about 3% to about 20%, or from about 5% to about 15% expression in respiratory endothelial cells in which the loaded LNP is accumulated.

In some embodiments, provided herein is a loaded LNP comprising:
(a) a lipid loaded LNP core,
(b) a therapeutic and/or prophylactic agent encapsulated within the core for delivery into a cell, and
(c) a cationic agent (e.g., a cationic lipid of Formula (I)),
   wherein the loaded LNP exhibits protein expression in from about 0.5% to about 50% respiratory endothelial cells in which the loaded LNP is accumulated. In some embodiments, the loaded LNP exhibits protein expression of from about 0.1% to about 60%, from about 0.5% to about 40%, from about 1% to about 30%, or from about 1% to about 20% of respiratory endothelial cells in which the loaded LNP is accumulated.

In some embodiments, provided herein is a loaded LNP comprising:
(a) a lipid loaded LNP core,
(b) a therapeutic and/or prophylactic agent encapsulated within the core for delivery into a cell, and
(c) a cationic agent (e.g., a cationic lipid of Formula (I)),
   wherein the loaded LNP exhibits protein expression in about 0.5% to about 50% of HeLa cells in which the loaded LNP is accumulated. In some embodiments, the loaded LNP exhibits protein expression of from about 0.1% to about 60%, from about 0.5% to about 40%, from about 1% to about 30%, or from about 1% to about 20% of HeLa cells in which the loaded LNP is accumulated.

In some embodiments, provided herein is a loaded LNP comprising:
(a) a lipid loaded LNP core,
(b) a therapeutic and/or prophylactic agent encapsulated within the core for delivery into a cell, and
(c) a cationic agent (e.g., a cationic lipid of Formula (I)),
   wherein the loaded LNP exhibits a cellular accumulation of at least about 20% in bronchial endothelial cells in a population of bronchial endothelial cells to which the loaded LNP is administered and exhibits about 5% or greater expression in bronchial endothelial cells in which the loaded LNP is accumulated. In some embodiments, the loaded LNP exhibits a cellular accumulation of from about 1% to about 75%, from about 5% to about 50%, about 10% to about 40%, or from about 15% to about 25% of respiratory endothelial cells in a population of respiratory endothelial cells to which the loaded LNP is administered. In some embodiments, the loaded LNP exhibits from about 0.5% to about 50%, from about 1% to about 40%, from about 3% to about 20%, or from about 5% to about 15% expression in lung endothelial cells in which the loaded LNP is accumulated.

In some embodiments, a molar ratio of the cationic agent to the therapeutic and/or prophylactic agent is about 0.1:1 to about 20:1. In some embodiments, a molar ratio of the cationic agent to the therapeutic and/or prophylactic agent is about 1.5:1 to about 10:1. In some embodiments, a molar ratio of the cationic agent to the therapeutic and/or prophylactic agent is about 1.5:1 to about 9:1. In some embodiments, a molar ratio of the cationic agent to the therapeutic and/or prophylactic agent is about 1.5:1 to about 8:1. In some embodiments, a molar ratio of the cationic agent to the therapeutic and/or prophylactic agent is about 1.5:1 to about 7:1. In some embodiments, a molar ratio of the cationic agent to the therapeutic and/or prophylactic agent is about 1.5:1 to about 6:1. In some embodiments, a molar ratio of the cationic agent to the therapeutic and/or prophylactic agent is about 1.5:1 to about 5:1. In some embodiments, a molar ratio of the cationic agent to the therapeutic and/or prophylactic agent is about 1.5:1. In some embodiments, a molar ratio of the cationic agent to the therapeutic and/or prophylactic agent is about 2:1. In some embodiments, a molar ratio of the cationic agent to the therapeutic and/or prophylactic agent is about 3:1. In some embodiments, a molar ratio of the cationic agent to the therapeutic and/or prophylactic agent is about 4:1. In some embodiments, a molar ratio of the cationic agent to the therapeutic and/or prophylactic agent is about 5:1.

In some embodiments, a nanoparticle of the disclosure (e.g., an empty LNP or loaded LNP) has a zeta potential of about 5 mV to about 20 mV. In some embodiments, the nanoparticle has a zeta potential of about 5 mV to about 15 mV. In some embodiments, the nanoparticle has a zeta potential of about 5 mV to about 12 mV. In some embodiments, the nanoparticle has a zeta potential of about 5 mV to about 10 mV.

### Cationic Agent

The cationic agent can comprise any aqueous/organic soluble molecule or substance that has a net positive charge. Such agent may also be lipid soluble but may also be soluble in aqueous solution. The cationic agent can be charged at physiologic pH. Physiological pH is the pH level normally observed in the human body. Physiological pH can be about 7.30-7.45 or about 7.35-7.45. Physiological pH can be about 7.40. Generally speaking, the cationic agent features a net positive charge at physiologic pH because it contains one or more basic functional groups that are protonated at physiologic pH in aqueous media. For example, the cationic agent can contain one or more amine groups, e.g. primary, secondary, or tertiary amines each having a pKa of 8.0 or greater. The pKa can be greater than about 9. In some embodiments, the cationic agent is a cationic lipid (i.e., a lipid that has a positive or partial positive charge at physiological pH). In some embodiments, the cationic agent is a cationic lipid of Formula (I).

In some embodiments, the lipids of Formula (I), include one or more of the following features when applicable.

In some embodiments, R′^{x} is:

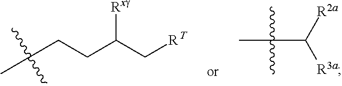

R′^{y} is:

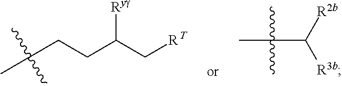

R′^{z} is:

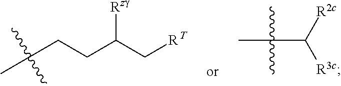

wherein

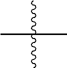

denotes a point of attachment;
R^{zγ} is selected from the group consisting of H, C₁₋₁₂ alkyl, and C₂₋₁₂ alkenyl;
R^{yγ} Tis selected from the group consisting of H, C₁₋₁₂ alkyl, and C₂₋₁₂ alkenyl;
R^{zγ} is selected from the group consisting of H, C₁₋₁₂ alkyl, and C₂₋₁₂ alkenyl; and
R^{2a}, R^{2b}, R^{2c}, R^{3a}, R^{3b}, and R^{3c} are each independently selected from the group consisting of C₁₋₁₄ alkyl and C₂₋₁₄ alkenyl.

In some embodiments,
R′^{a} is:

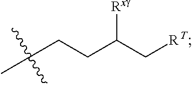

R′^{b} is:

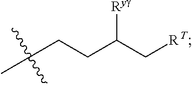

and R′^{c} is:

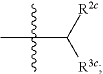

wherein

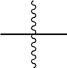

denotes a point of attachment;
R^{xγ} is selected from the group consisting of H, C₁₋₁₂ alkyl, and C₂₋₁₂ alkenyl;
R^{yγ} is selected from the group consisting of H, C₁₋₁₂ alkyl, and C₂₋₁₂ alkenyl; and
R^{2c} and R^{3c} are each independently selected from the group consisting of C₁₋₁₄ alkyl and C₂₋₁₄ alkenyl.

In some embodiments, R^{xγ} and R^{yγ} are each H.

In some embodiments, R^{xγ} and R^{yγ} are each C₁₋₁₂ alkyl or C₂₋₁₂ alkenyl.

In some embodiments, R^{xγ} is C₁₋₁₂ alkyl or C₂₋₁₂ alkenyl, and R^{yγ} is H.

In some embodiments, q is 2.

In some embodiments the cationic lipid of Formula (I) described herein is suitable for making a nanoparticle composition for intramuscular administration.

In some embodiments, the cationic lipid of Formula (I) is selected from the lipids of Table 1 or an isomer thereof.

TABLE 1
Cationic Lipids.

| Cpd | Structure |
|---|---|
| 1 | 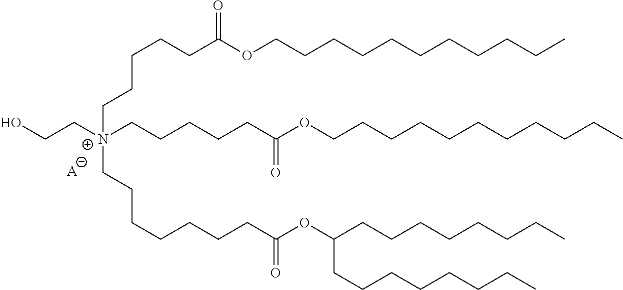 |
| 2 | 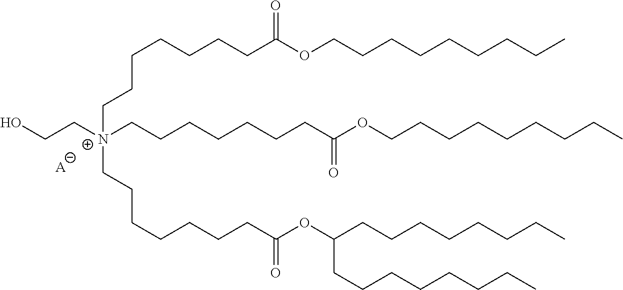 |
| 3 | 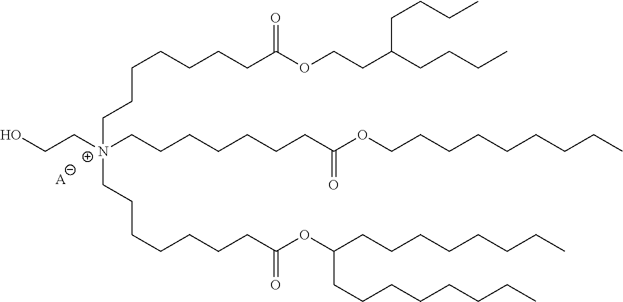 |
| 4 | 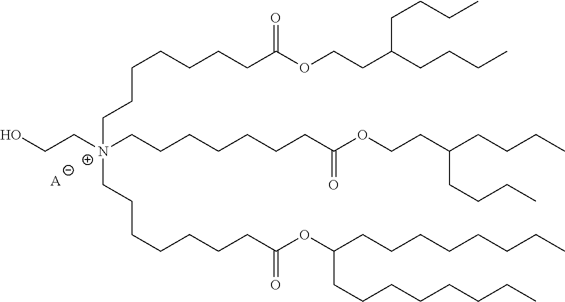 |

wherein A⁻ is bromide or hydroxide.

### Definitions

As used herein, the term “alkyl” or “alkyl group” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The notation “C₁₋₁₄ alkyl” means an optionally substituted linear or branched, saturated hydrocarbon including 1-14 carbon atoms. Unless otherwise specified, an alkyl group described herein refers to both unsubstituted and substituted alkyl groups. As used herein, a “linear” alkyl means a straight chain alkyl (methyl, ethyl, n-propyl, n-butyl, n-pentyl, n-hexyl, n-heptyl, n-octyl, n-nonyl, n-decyl, n-undecyl or n-dodecyl), wherein the attachment point is at the C₁ carbon.

As used herein, the term “alkenyl” or “alkenyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The notation “C₂₋₁₄ alkenyl” means an optionally substituted linear or branched hydrocarbon including 2-14 carbon atoms and at least one carbon-carbon double bond. An alkenyl group may include one, two, three, four, or more carbon-carbon double bonds. For example, C₁₈ alkenyl may include one or more double bonds. A C₁₈ alkenyl group including two double bonds may be a linoleyl group. Unless otherwise specified, an alkenyl group described herein refers to both unsubstituted and substituted alkenyl groups.

As used herein, the term “alkynyl” or “alkynyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one carbon-carbon triple bond, which is optionally substituted. The notation “C₂₋₁₄ alkynyl” means an optionally substituted linear or branched hydrocarbon including 2-14 carbon atoms and at least one carbon-carbon triple bond. An alkynyl group may include one, two, three, four, or more carbon-carbon triple bonds. For example, C₁₈ alkynyl may include one or more carbon-carbon triple bonds. Unless otherwise specified, an alkynyl group described herein refers to both unsubstituted and substituted alkynyl groups.

As used herein, the term “carbocycle” or “carbocyclic group” means an optionally substituted mono- or multi-cyclic system including one or more rings of carbon atoms. Rings may be three, four, five, six, seven, eight, nine, ten, eleven, twelve, thirteen, fourteen, fifteen, sixteen, seventeen, eighteen, nineteen, or twenty membered rings. The notation “C₃₋₆ carbocycle” means a carbocycle including a single ring having 3-6 carbon atoms. Carbocycles may include one or more carbon-carbon double or triple bonds and may be non-aromatic or aromatic (e.g., cycloalkyl or aryl groups). A carbocycle may be a mono- or multi-ring (e.g., fused, bridged, or spiro rings) system. Examples of carbocycles include cyclopropyl, cyclopentyl, cyclohexyl, phenyl, naphthyl, and 1,2-dihydronaphthyl groups. The term “cycloalkyl” as used herein means a non-aromatic carbocycle and may or may not include any double or triple bond. Unless otherwise specified, carbocycles described herein refers to both unsubstituted and substituted carbocycle groups, i.e., optionally substituted carbocycles. In some embodiments, the carbocycle is a C₃₋₈ cycloalkyl. In some embodiments, the carbocycle is a C₃₋₆ cycloalkyl. In some embodiments, the carbocycle is a C₆₋₁₀ aryl.

“Aryl” includes groups with aromaticity, including “conjugated,” or multicyclic systems with at least one aromatic ring and do not contain any heteroatom in the ring structure. Examples include phenyl, benzyl, 1,2,3,4-tetrahydronaphthalenyl, etc. In some embodiments, an “aryl” is a C₆₋₁₀ carbocycle with aromatity (e.g., an “aryl” is a C₆₋₁₀ aryl).

As used herein, the term “heterocycle” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. The term “heterocycloalkyl” as used herein means a non-aromatic heterocycle and may or may not include any double or triple bond. Unless otherwise specified, heterocycles described herein refers to both unsubstituted and substituted heterocycle groups, i.e., optionally substituted heterocycles. In some embodiments, the heterocycle is a 4 to 12-membered heterocycloalkyl. In some embodiments, the heterocycle is a 5- or 6-membered heteroaryl.

“Heteroaryl” groups are aryl groups, as defined above, except having from one to four heteroatoms in the ring structure, and may also be referred to as “aryl heterocycles” or “heteroaromatics.” As used herein, the term “heteroaryl” is intended to include a stable 5-, 6- or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen sulfur, and boron. The nitrogen atom may be substituted or unsubstituted (i.e., N or NR wherein R is H or other substituents, as defined). The nitrogen and sulfur heteroatoms may optionally be oxidized (i.e., N→O and S(O)ₚ, where p=1 or 2).

Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

As used herein, a “biodegradable group” is a group that may facilitate faster metabolism of a lipid in a mammalian entity. A biodegradable group may be selected from the group consisting of, but is not limited to, —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group. As used herein, an “aryl group” is an optionally substituted carbocyclic group including one or more aromatic rings. Examples of aryl groups include phenyl and naphthyl groups. As used herein, a “heteroaryl group” is an optionally substituted heterocyclic group including one or more aromatic rings. Examples of heteroaryl groups include pyrrolyl, furyl, thiophenyl, imidazolyl, oxazolyl, and thiazolyl. Both aryl and heteroaryl groups may be optionally substituted. For example, M and M′ can be selected from the non-limiting group consisting of optionally substituted phenyl, oxazole, and thiazole. In the formulas herein, M and M′ can be independently selected from the list of biodegradable groups above. Unless otherwise specified, aryl or heteroaryl groups described herein refers to both unsubstituted and substituted groups, i.e., optionally substituted aryl or heteroaryl groups.

Alkyl, alkenyl, and cyclyl (e.g., carbocyclyl and heterocyclyl) groups may be optionally substituted unless otherwise specified. Optional substituents may be selected from the group consisting of, but are not limited to, a halogen atom (e.g., a chloride, bromide, fluoride, or iodide group), a carboxylic acid (e.g., —C(O)OH), an alcohol (e.g., a hydroxyl, —OH), an ester (e.g., —C(O)OR or —OC(O)R), an aldehyde (e.g., —C(O)H), a carbonyl (e.g., —C(O)R, alternatively represented by C═O), an acyl halide (e.g., —C(O)X, in which X is a halide selected from bromide, fluoride, chloride, and iodide), a carbonate (e.g., —OC(O)OR), an alkoxy (e.g., —OR), an acetal (e.g., —C(OR)₂R″″, in which each OR are alkoxy groups that can be the same or different and R″″ is an alkyl or alkenyl group), a phosphate (e.g., P(O)₄³⁻), a thiol (e.g., —SH), a sulfoxide (e.g., —S(O)R), a sulfinic acid (e.g., —S(O)OH), a sulfonic acid (e.g., —S(O)₂OH), a thial (e.g., —C(S)H), a sulfate (e.g., S(O)₄²⁻), a sulfonyl (e.g., —S(O)₂—), an amide (e.g., —C(O)NR₂, or —N(R)C(O)R), an azido (e.g., —N₃), a nitro (e.g., —NO₂), a cyano (e.g., —CN), an isocyano (e.g., —NC), an acyloxy (e.g., —OC(O)R), an amino (e.g., —NR₂, —NRH, or —NH₂), a carbamoyl (e.g., —OC(O)NR₂, —OC(O)NRH, or —OC(O)NH₂), a sulfonamide (e.g., —S(O)₂NR₂, —S(O)₂NRH, —S(O)₂NH₂, —N(R)S(O)₂R, —N(H)S(O)₂R, —N(R)S(O)₂H, or —N(H)S(O)₂H), an alkyl group, an alkenyl group, and a cyclyl (e.g., carbocyclyl or heterocyclyl) group. In any of the preceding, R is an alkyl or alkenyl group, as defined herein. In some embodiments, the substituent groups themselves may be further substituted with, for example, one, two, three, four, five, or six substituents as defined herein. For example, a C₁₋₆ alkyl group may be further substituted with one, two, three, four, five, or six substituents as described herein.

Lipids of the disclosure that contain nitrogens can be converted to N-oxides by treatment with an oxidizing agent (e.g., 3-chloroperoxybenzoic acid (mCPBA) and/or hydrogen peroxides) to afford other lipids of the disclosure. Thus, all shown and claimed nitrogen-containing lipids are considered, when allowed by valency and structure, to include both the lipid as shown and its N-oxide derivative (which can be designated as N→O or N⁺—O⁻). Furthermore, in other instances, the nitrogens in the lipids of the disclosure can be converted to N-hydroxy or N-alkoxy lipids. For example, N-hydroxy lipids can be prepared by oxidation of the parent amine by an oxidizing agent such as m-CPBA. All shown and claimed nitrogen-containing lipids are also considered, when allowed by valency and structure, to cover both the lipid as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

About, Approximately: As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11, 1%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given lipid in a lipid component of a nanoparticle composition, “about” may mean +/−10% of the recited value. For instance, a nanoparticle composition including a lipid component having about 40% of a given lipid may include 30-50% of the lipid.

As used herein, the term “lipid,” is meant to include all isomers and isotopes of the structure depicted. “Isotopes” refers to atoms having the same atomic number but different mass numbers resulting from a different number of neutrons in the nuclei. For example, isotopes of hydrogen include tritium and deuterium. Further, a lipid, salt, or complex of the present disclosure can be prepared in combination with solvent or water molecules to form solvates and hydrates by routine methods.

As used herein, the term “contacting” means establishing a physical connection between two or more entities. For example, contacting a mammalian cell with a nanoparticle composition means that the mammalian cell and a nanoparticle are made to share a physical connection. Methods of contacting cells with external entities both in vivo and ex vivo are well known in the biological arts. For example, contacting a nanoparticle composition and a mammalian cell disposed within a mammal may be performed by varied routes of administration (e.g., intravenous, intramuscular, intradermal, and subcutaneous) and may involve varied amounts of lipid nanoparticles (e.g., empty LNPs or loaded LNPs). Moreover, more than one mammalian cell may be contacted by a nanoparticle composition.

As used herein, the term “delivering” means providing an entity to a destination. For example, delivering a therapeutic and/or prophylactic to a subject may involve administering a nanoparticle composition including the therapeutic and/or prophylactic to the subject (e.g., by an intravenous, intramuscular, intradermal, or subcutaneous route). Administration of a nanoparticle composition to a mammal or mammalian cell may involve contacting one or more cells with the nanoparticle composition.

As used herein, the term “enhanced delivery” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target tissue of interest (e.g., mammalian liver or lung) compared to the level of delivery of a therapeutic and/or prophylactic by a control nanoparticle to a target tissue of interest (e.g., MC3, KC2, or DLinDMA). The level of delivery of a nanoparticle to a particular tissue may be measured by comparing the amount of protein produced in a tissue to the weight of said tissue, comparing the amount of therapeutic and/or prophylactic in a tissue to the weight of said tissue, comparing the amount of protein produced in a tissue to the amount of total protein in said tissue, or comparing the amount of therapeutic and/or prophylactic in a tissue to the amount of total therapeutic and/or prophylactic in said tissue. It will be understood that the enhanced delivery of a nanoparticle to a target tissue need not be determined in a subject being treated, it may be determined in a surrogate such as an animal model (e.g., a rat model). In certain embodiments, a nanoparticle composition including a cationic lipid of the disclosure has substantively the same level of delivery enhancement regardless of administration routes. For example, certain lipids disclosed herein exhibit similar delivery enhancement when they are used for delivering a therapeutic and/or prophylactic either intravenously or intramuscularly. In other embodiments, certain lipids disclosed herein exhibit a higher level of delivery enhancement when they are used for delivering a therapeutic and/or prophylactic intramuscularly than intravenously.

As used herein, the term “specific delivery,” “specifically deliver,” or “specifically delivering” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target tissue of interest (e.g., mammalian liver or lung) compared to an off-target tissue (e.g., mammalian spleen). The level of delivery of a nanoparticle to a particular tissue may be measured by comparing the amount of protein produced in a tissue to the weight of said tissue, comparing the amount of therapeutic and/or prophylactic in a tissue to the weight of said tissue, comparing the amount of protein produced in a tissue to the amount of total protein in said tissue, or comparing the amount of therapeutic and/or prophylactic in a tissue to the amount of total therapeutic and/or prophylactic in said tissue. For example, for renovascular targeting, a therapeutic and/or prophylactic is specifically provided to a mammalian kidney as compared to the liver and spleen if 1.5, 2-fold, 3-fold, 5-fold, 10-fold, 15 fold, or 20 fold more therapeutic and/or prophylactic per 1 g of tissue is delivered to a kidney compared to that delivered to the liver or spleen following systemic administration of the therapeutic and/or prophylactic. It will be understood that the ability of a nanoparticle to specifically deliver to a target tissue need not be determined in a subject being treated, it may be determined in a surrogate such as an animal model (e.g., a rat model).

As used herein, “encapsulation efficiency” refers to the amount of a therapeutic and/or prophylactic that becomes part of a nanoparticle composition, relative to the initial total amount of therapeutic and/or prophylactic used in the preparation of a nanoparticle composition. For example, if 97 mg of therapeutic and/or prophylactic are encapsulated in a nanoparticle composition out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

As used herein, “encapsulation”, “encapsulated”, “loaded”, and “associated” may refer to complete, substantial, or partial enclosure, confinement, surrounding, or encasement. As used herein, “encapsulation” or “association” may refer to the process of confining an individual nucleic acid molecule within a nanoparticle and/or establishing a physiochemical relationship between an individual nucleic acid molecule and a nanoparticle. As used herein, an “empty nanoparticle” may refer to a nanoparticle that is substantially free of a therapeutic or prophylactic agent. As used herein, an “empty nanoparticle” or an “empty lipid nanoparticle” may refer to a nanoparticle that is substantially free of a nucleic acid. As used herein, an “empty nanoparticle” or an “empty lipid nanoparticle” may refer to a nanoparticle that is substantially free of a nucleotide or a polypeptide. As used herein, an “empty nanoparticle” or an “empty lipid nanoparticle” may refer to a nanoparticle that consists substantially of only lipid components. As used herein, a “loaded LNP”, “loaded nanoparticle” or a “loaded lipid nanoparticle” (also referred to as a “full nanoparticle” or a “full lipid nanoparticle”) may refer to a nanoparticle comprising the components of the empty nanoparticle, and a substantial amount of a therapeutic or prophylactic agent. In some embodiments, the loaded LNP comprises a therapeutic or prophylactic agent that is at least partially in the interior of the LNP. In some embodiments, the loaded LNP comprises a substantial amount of a therapeutic or prophylactic agent that is associated with the surface of the LNP or conjugated to the exterior of the LNP. As used herein, a “loaded LNP As used herein, a “loaded LNP”, “loaded nanoparticle” or a “loaded lipid nanoparticle” (also referred to as a “full nanoparticle” or a “full lipid nanoparticle”) may refer to a nanoparticle comprising the components of the empty nanoparticle, and a substantial amount of a nucleotide or polypeptide. In some embodiments, the loaded LNP comprises a nucleotide or polypeptide that is at least partially in the interior of the LNP. In some embodiments, the loaded LNP comprises a nucleotide or polypeptide that is associated with the surface of the LNP or conjugated to the exterior of the LNP. As used herein, a “loaded LNP”, “loaded nanoparticle” or a “loaded lipid nanoparticle” (also referred to as a “full nanoparticle” or a “full lipid nanoparticle”) may refer to a nanoparticle comprising the components of the empty nanoparticle, and a substantial amount of a nucleic acid. In some embodiments, the loaded LNP comprises a nucleic acid (e.g., an mRNA) that is at least partially in the interior of the LNP. In some embodiments, the loaded LNP comprises nucleic acid (e.g., an mRNA) that is associated with the surface of the LNP or conjugated to the exterior of the LNP.

As used herein, “expression” of a nucleic acid sequence refers to translation of an mRNA into a polypeptide or protein and/or post-translational modification of a polypeptide or protein.

As used herein “expression of a loaded LNP” refers to expression by an agent comprised in the loaded LNP. For example, in some embodiments, the agent is a nucleic acid (e.g. an mRNA). For example, in some embodiments, the agent expresses a protein or a polypeptide. In some embodiments, the protein is a fluorescent protein. For example, in some embodiments, the term “the loaded LNP exhibits expression” means that a loaded LNP accumulated in a cell delivers an agent to the cell, and that said agent (e.g., a nucleic acid, e.g. an mRNA) expresses e.g., a protein or polypeptide in the cell.

As used herein “hydrophobicity” of a lipid describes the tendency of a lipid to exclude water. In some embodiments, the hydrophobicity of a lipid nanoparticle surface impacts the penetration of a lipid nanoparticle across the lipid bilayer of a cell. In some embodiments, hydrophobic nanoparticles show increased cellular uptake relative to hydrophilic lipid nanoparticles.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

As used herein, the term “isomer” means any geometric isomer, tautomer, zwitterion, stereoisomer, enantiomer, or diastereomer of a compound (e.g., a lipid of the disclosure). Compounds compound (e.g., lipids of the disclosure) may include one or more chiral centers and/or double bonds and may thus exist as stereoisomers, such as double-bond isomers (i.e., geometric E/Z isomers) or diastereomers (e.g., enantiomers (i.e., (+) or (−)) or cis/trans isomers). The present disclosure encompasses any and all isomers of the lipids described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of lipids and means of resolving them into their component enantiomers or stereoisomers are well-known.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerization is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine. An example of tautomerism in di-substituted guanidine is shown below.

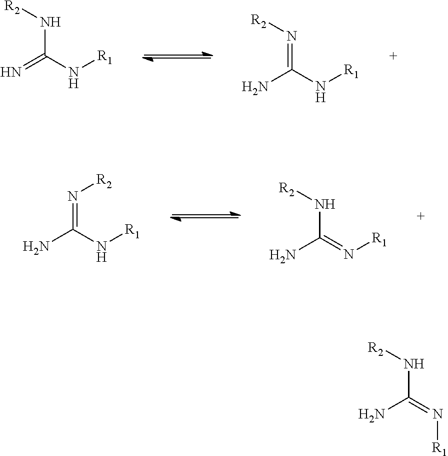

It is to be understood that the lipids of the disclosure may be depicted as different tautomers. It should also be understood that when lipids have tautomeric forms, all tautomeric forms are intended to be included in the scope of the disclosure, and the naming of the lipids does not exclude any tautomer form.

As used herein, a “lipid component” is that component of a nanoparticle composition that includes one or more lipids. For example, the lipid component may include one or more cationic lipids, ionizable lipids, PEGylated, structural, or other lipids, such as phospholipids. In some embodiments of the empty LNP or loaded LNP of the disclosure, the lipid component comprises at least one cationic lipid and at least one ionizable lipid.

As used herein, a “linker” is a moiety connecting two moieties, for example, the connection between two nucleosides of a cap species. A linker may include one or more groups including but not limited to phosphate groups (e.g., phosphates, boranophosphates, thiophosphates, selenophosphates, and phosphonates), alkyl groups, amidates, or glycerols. For example, two nucleosides of a cap analog may be linked at their 5′ positions by a triphosphate group or by a chain including two phosphate moieties and a boranophosphate moiety.

As used herein, “methods of administration” may include intravenous, intramuscular, intradermal, subcutaneous, or other methods of delivering a composition to a subject. A method of administration may be selected to target delivery (e.g., to specifically deliver) to a specific region or system of a body.

As used herein, “modified” means non-natural. For example, an RNA may be a modified RNA. That is, an RNA may include one or more nucleobases, nucleosides, nucleotides, or linkers that are non-naturally occurring. A “modified” species may also be referred to herein as an “altered” species. Species may be modified or altered chemically, structurally, or functionally. For example, a modified nucleobase species may include one or more substitutions that are not naturally occurring.

As used herein, the “N:P ratio” is the molar ratio of ionizable (in the physiological pH range) nitrogen atoms in a lipid to phosphate groups in an RNA, e.g., in a nanoparticle composition including a lipid component and an RNA.

As used herein, a “nanoparticle composition” is a composition comprising one or more lipids. Nanoparticle compositions are typically sized on the order of micrometers or smaller and may include a lipid bilayer. Nanoparticle compositions encompass lipid nanoparticles (LNPs), liposomes (e.g., lipid vesicles), and lipoplexes. For example, a nanoparticle composition may be a liposome having a lipid bilayer with a diameter of 500 nm or less.

As used herein, “naturally occurring” means existing in nature without artificial aid.

As used herein, “patient” refers to a subject who may seek or be in need of treatment, requires treatment, is receiving treatment, will receive treatment, or a subject who is under care by a trained professional for a particular disease or condition.

As used herein, a “PEG lipid” or “PEGylated lipid” refers to a lipid comprising a polyethylene glycol component.

The phrase “pharmaceutically acceptable” is used herein to refer to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient,” as used herein, refers to any ingredient other than the lipids described herein (for example, a vehicle capable of suspending, complexing, or dissolving the active lipid) and having the properties of being substantially nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, xylitol, and other species disclosed herein.

In the present specification, the structural formula of the lipid represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. In addition, a crystal polymorphism may be present for the lipids represented by the formula. It is noted that any crystal form, crystal form mixture, or anhydride or hydrate thereof is included in the scope of the present disclosure.

The term “crystal polymorphs”, “polymorphs” or “crystal forms” means crystal structures in which a compound (e.g., a lipid of the disclosure; or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the lipids can be prepared by crystallization under different conditions.

The compounds of the present disclosure include the compounds themselves, as well as their salts and their solvates, if applicable. A salt, for example, can be formed between an anion and a positively charged group, e.g., a quaternary amino group. Suitable anions include chloride, bromide, iodide, sulfate, bisulfate, sulfamate, nitrate, phosphate, citrate, hydroxide, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. In the salt form, it is understood that the ratio of the compound to the cation or anion of the salt can be 1:1, or any ratio other than 1:1, e.g., 3:1, 2:1, 1:2, or 1:3.

Compositions may also include salts of one or more lipids. Salts may be pharmaceutically acceptable salts. As used herein, “pharmaceutically acceptable salts” refers to derivatives of the disclosed lipids wherein the parent lipid is altered by converting an existing acid or base moiety to its salt form (e.g., by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent lipid formed, for example, from non-toxic inorganic or organic acids. The pharmaceutically acceptable salts of the present disclosure can be synthesized from the parent lipid which contains a basic or acidic moiety by conventional chemical methods. Generally, such salts can be prepared by reacting the free acid or base forms of these lipids with a stoichiometric amount of the appropriate base or acid in water or in an organic solvent, or in a mixture of the two; generally, nonaqueous media like ether, ethyl acetate, ethanol, isopropanol, or acetonitrile are preferred.

It is understood that the compounds of the present disclosure include the compounds themselves, as well as their ionic derivatives, if applicable. As used herein, the term “ionic derivative” refers to an ionic form of the referenced structure. The ionic form may be a free cation, a free anion, or a zwitterion. For example, when a structure is described herein as a salt, the disclosure intends to include the corresponding free cation of the structure, or the corresponding free anion of the structure. For example, a compound of Formula (I):

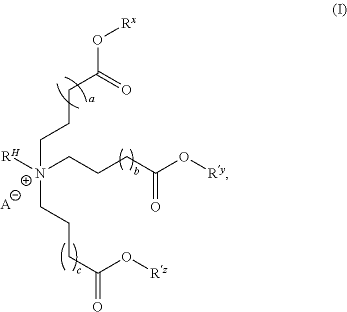

intends to include its corresponding free cation:

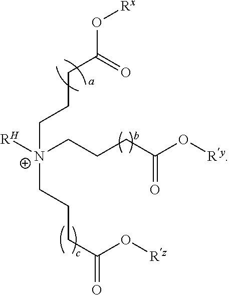

As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

As used herein, the “polydispersity index,” or “PDI” is a ratio that describes the homogeneity of the particle size distribution of a system. A small value, e.g., less than 0.3, indicates a narrow particle size distribution.

As used herein, the term “polypeptide” or “polypeptide of interest” refers to a polymer of amino acid residues typically joined by peptide bonds that can be produced naturally (e.g., isolated or purified) or synthetically. The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. The term, as used herein, refers to proteins, polypeptides, and peptides of any size, structure, or function. Polypeptides include encoded polynucleotide products, naturally occurring polypeptides, synthetic polypeptides, homologs, orthologs, paralogs, fragments and other equivalents, variants, and analogs of the foregoing. A polypeptide can be a monomer or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some embodiments, a “peptide” can be less than or equal to 50 amino acids long, e.g., about 5, 10, 15, 20, 25, 30, 35, 40, 45, or 50 amino acids long.

As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest.

As used herein, a “DNA” refers to a desoxyribonucleic acid that may be naturally or non-naturally occurring. For example, a DNA may be a synthetic molecule, e.g., a synthetic DNA molecule produced in vitro. In some embodiments, the DNA molecule is a recombinant molecule. As used herein, a “recombinant DNA molecule” refers to a DNA molecule that does not exist as a natural product, but is produced using molecular biology techniques.

As used herein, a “single unit dose” is a dose of any therapeutic administered in one dose/at one time/single route/single point of contact, i.e., single administration event.

As used herein, a “split dose” is the division of single unit dose or total daily dose into two or more doses.

As used herein, a “total daily dose” is an amount given or prescribed in 24 hour period. It may be administered as a single unit dose.

As used herein, “size” or “mean size” in the context of lipid nanoparticles (e.g., empty LNPs or loaded LNPs) refers to the mean diameter of a nanoparticle composition.

As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

As used herein, “targeted cells” refers to any one or more cells of interest. The cells may be found in vitro, in vivo, in situ, or in the tissue or organ of an organism. The organism may be an animal, preferably a mammal, more preferably a human and most preferably a patient.

As used herein “target tissue” refers to any one or more tissue types of interest in which the delivery of a therapeutic and/or prophylactic would result in a desired biological and/or pharmacological effect. Examples of target tissues of interest include specific tissues, organs, and systems or groups thereof. In particular applications, a target tissue may be a kidney, a lung, a spleen, vascular endothelium in vessels (e.g., intra-coronary or intra-femoral), or tumor tissue (e.g., via intratumoral injection). An “off-target tissue” refers to any one or more tissue types in which the expression of the encoded protein does not result in a desired biological and/or pharmacological effect. In particular applications, off-target tissues may include the liver and the spleen.

The term “therapeutic agent” or “prophylactic agent” refers to any agent that, when administered to a subject, has a therapeutic, diagnostic, and/or prophylactic effect and/or elicits a desired biological and/or pharmacological effect. Therapeutic agents are also referred to as “actives” or “active agents.” Such agents include, but are not limited to, cytotoxins, radioactive ions, chemotherapeutic agents, small molecule drugs, proteins, and nucleic acids.

As used herein, the term “therapeutically effective amount” means an amount of an agent to be delivered (e.g., nucleic acid, drug, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

As used herein, “transfection” refers to the introduction of a species (e.g., an RNA) into a cell. Transfection may occur, for example, in vitro, ex vivo, or in vivo.

As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the “zeta potential” is the electrokinetic potential of a lipid, e.g., in a particle composition.

### Nanoparticle Compositions

The disclosure also features lipid nanoparticles (e.g., an empty LNP or a loaded LNP) comprising a cationic lipid according to Formula (I) as described herein.

In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter), e.g., when measured by dynamic light scattering (DLS), transmission electron microscopy, scanning electron microscopy, or another method. Nanoparticle compositions include, for example, lipid nanoparticles (LNPs; e.g., empty LNPs or loaded LNPs), liposomes, lipid vesicles, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers may be functionalized and/or cross-linked to one another. Lipid bilayers may include one or more ligands, proteins, or channels.

A lipid nanoparticle (e.g., an empty LNP or a loaded LNP) of the present disclosure comprise at least one cationic lipid according to Formula (I). In some embodiments, an empty LNP or a loaded LNP of the disclosure includes one or more of cationic lipids of Table 1. Nanoparticle compositions may also include a variety of other components. For example, in some embodiments, the empty LNP or a loaded LNP includes one or more ionizable lipids in addition to a lipid according to Formula (I).

### Ionizable Lipids

In some embodiments, a lipid nanoparticle (e.g., an empty LNP or a loaded LNP) of the disclosure further includes one or more ionizable lipids in addition to the cationic lipid of Formula (I).

In some aspects, the ionizable lipid is of compound of Formula (IL-A):

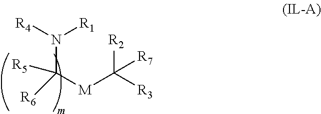

### or its N-oxide,

or a salt or isomer thereof, wherein:
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of hydrogen, a C₃₋₆ carbocycle, —(CH₂)ₙQ, —(CH₂)ₙCHQR, —(CH₂)ₒC(R¹²)₂(CH₂)ₙ₋ₒQ, —CHQR, —CQ(R)₂, —C(O)NQR and unsubstituted C₁₋₆ alkyl, where Q is selected from a carbocycle, heterocycle, —OR, —O(CH₂)ₙN(R)₂, —C(O)OR, —OC(O)R, —OC(O)O—, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R′, —N(R)S(O)₂R⁸, —O(CH₂)ₙOR, —N(R)C(═NR₉)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, —(CH₂)ₙN(R)₂ and —C(R)N(R)₂C(O)OR, NR^{A}S(O)₂R^{SX}, and

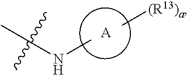

wherein A is a 3-14 membered heterocycle containing one or more heteroatoms selected from N, O and S; and a is 1, 2, 3, or 4; wherein

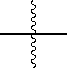

denotes a point of attachment;
each o is independently selected from 1, 2, 3, and 4, and each n is independently selected from 1, 2, 3, 4, and 5;
R⁸ is selected from the group consisting of C₃₋₆ carbocycle and heterocycle;
R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
R¹² is selected from the group consisting of H, OH, C₁₋₃ alkyl, and C₂₋₃ alkenyl;
each R is independently selected from the group consisting of C₁₋₆ alkyl, C₁₋₃ alkyl-aryl, C₂₋₃ alkenyl, and H;
R^{A} is selected from H and C₁₋₃ alkyl;
R^{SX} is selected from a C₃₋₈ carbocycle, a 3-14 membered heterocycle containing one or more heteroatoms selected from N, O and S, C₁₋₆ alkyl, C₂₋₆ alkenyl, (C₁₋₃ alkoxy)C₁₋₃ alkyl, (CH₂)ₚ₁O(CH₂)ₚ₂R^{SX1}, and (CH₂)ₚ₁R^{SX1}, wherein the carbocycle and heterocycle are optionally substituted with one or more groups selected from oxo, C₁₋₆ alkyl, and (C₁₋₃ alkoxy)C₁₋₃ alkyl;
R^{SX1} is selected from C(O)NR¹⁴R¹⁴′, a C₃₋₈ carbocycle, and a 3-14 membered heterocycle containing one or more heteroatoms selected from N, O and S, wherein the carbocycle and heterocycle are each optionally substituted with one or more groups selected from oxo, halo, C₁₋₃ alkyl, (C₁₋₃ alkoxy)C₁₋₃ alkyl, C₁₋₆ alkylamino, di-(C₁₋₆ alkyl) amino, and NH₂;
each R¹³ is selected from the group consisting of OH, oxo, halo, C₁₋₆ alkyl, C₁₋₆ alkoxy, C₂₋₆ alkenyl, C₁₋₆ alkylamino, di-(C₁₋₆ alkyl) amino, NH₂, C(O)NH₂, CN, and NO₂;
R¹⁴ and R^{14′} are each independently selected from the group consisting of H and C₁₋₆ alkyl;
p₁ is selected from 1, 2, 3, 4, and 5;
p₂ is selected from 1, 2, 3, 4, and 5;
each R⁵ is independently selected from the group consisting of OH, C₁₋₃ alkyl, C₂₋₃ alkenyl, and H;
each R⁶ is independently selected from the group consisting of OH, C₁₋₃ alkyl, C₂₋₃ alkenyl, and H;
R⁷ is selected from the group consisting of C₁₋₃ alkyl, C₂₋₃ alkenyl, and H;
M and M′ are independently selected from —C(O)O—, —OC(O)—, —OC(O)O—, —OC(O)-M″-C(O)O—,
   —C(O)N(R^{M})—, —N(R^{M})C(O)—, —C(O)—, —C(S)—, —C(S)S—, —SC(S)—, —CH(OH)—, —P(O)(O R^{M})O—, —S(O)₂—, —S—S—, an aryl group, and a heteroaryl group, in which M″ is a bond, C₁₋₁₃ alkyl or C₂₋₁₃ alkenyl;
each R^{M} is independently selected from the group consisting of H, C₁₋₆ alkyl and C₂₋₆ alkenyl;
each R′ is independently selected from the group consisting of C₁₋₁₈ alkyl, C₂₋₁₈ alkenyl, —R*YR″, —YR″, (CH₂)_{q′}OR*, and H,
and each q′ is independently selected from 1, 2, and 3;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some aspects, the ionizable lipid is of compound of Formula (IL-B):

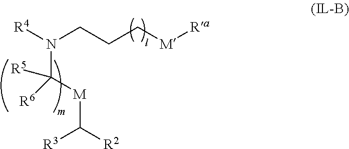

or its N-oxide, or a salt or isomer thereof,
wherein R′^{a} is R′^{branched}; wherein
R′^{branched} is:

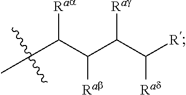

wherein

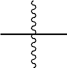

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)nOH, wherein n is selected from the group consisting of 1, 2, 3, 4, and 5, and

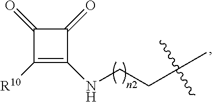

wherein

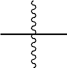

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some aspects, the ionizable lipid is of compound a compound of Formula (IL-C):

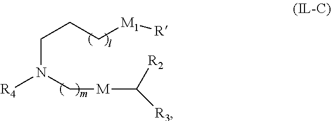

or a salt or isomer thereof, wherein
l is selected from 1, 2, 3, 4, and 5;
M₁ is M′;
R₄ is —(CH₂)ₙQ, in which Q is OH, and n is selected from 1, 2, 3, 4, or 5;
M and M′ are independently selected from —C(O)O— and —OC(O)—;
R₂ and R₃ are both C₁₋₁₄ alkyl, or C₂₋₁₄ alkenyl; and
R′ is a C₁-C₁₂ linear alkyl.

In some aspects, the ionizable lipid is of compound a compound of Formula (IL-D):

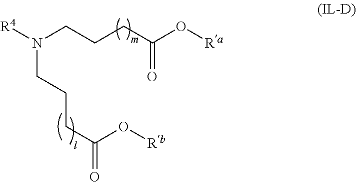

or its N-oxide, or a salt or isomer thereof,
wherein R′^{a} is R′^{branched} or R′^{cyclic}; wherein
R′^{branched} is:

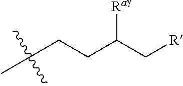

and R′b is:

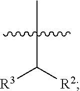

wherein

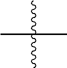

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some aspects, the ionizable lipid is a of compound of Formula (IL-I):

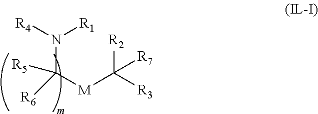

or its N-oxide, or a salt or isomer thereof, wherein:
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of hydrogen, a C₃₋₆ carbocycle, —(CH₂)ₙQ, —(CH₂)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;
each R⁵ is independently selected from the group consisting of OH, C₁₋₃ alkyl, C₂₋₃ alkenyl, and H;
each R⁶ is independently selected from the group consisting of OH, C₁₋₃ alkyl, C₂₋₃ alkenyl, and H;
M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R⁷ is selected from the group consisting of C₁₋₃ alkyl, C₂₋₃ alkenyl, and H;
R′ is selected from the group consisting of C₃₋₆ carbocycle and heterocycle;
R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
R¹⁰ is selected from the group consisting of H, OH, C₁₋₃ alkyl, and C₂₋₃ alkenyl;
each R is independently selected from the group consisting of C₁₋₃ alkyl, C₂₋₃ alkenyl, (CH₂)_{q}OR*, and H,
and each q is independently selected from 1, 2, and 3;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some aspects, the ionizable lipid is a of compound of Formula (IL-IA):

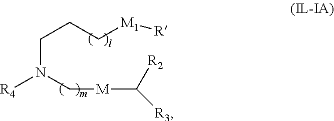

or a salt or isomer thereof, wherein
l is selected from 1, 2, 3, 4, and 5;
m is selected from 5, 6, 7, 8, and 9;
M¹ is a bond or M′;
R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, or heteroaryl, and each n is selected from 1, 2, 3, 4, or 5;
M and M′ are independently selected from —C(O)O—, —OC(O)—, —C(O)N(R′)—, —P(O)(OR′)O—, —S—S—, an aryl group, and a heteroaryl group; and
   R₂ and R₃ are both C₁₋₁₄ alkyl or C₂₋₁₄ alkenyl;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H; and
R′ is a C₁₋₁₈ alkyl or C₂₋₁₈ alkenyl.

In some aspects, the ionizable lipid is a compound of Formula (IL-IB):

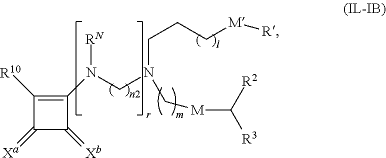

### or its N-oxide,

or a salt or isomer thereof, wherein
l is selected from 1, 2, 3, 4, and 5;
m is selected from 5, 6, 7, 8, and 9;
R′ is selected from the group consisting of C₁₋₁₄ alkyl and C₂₋₁₄ alkenyl; and
R² and R³ are independently selected from the group consisting of C₁₋₁₄ alkyl, and C₂₋₁₄ alkenyl;
M and M′ are independently selected from —C(O)O— and —OC(O)—;
R^{N} is H, or C₁₋₃ alkyl;
X^{a} and X^{b} are each independently O or S;
   R¹⁰ is selected from the group consisting of H, halo, —OH, R, —N(R)₂, —CN, —N₃, —C(O)OH, —C(O)OR, —OC(O)R, —OR, —SR, —S(O)R, —S(O)OR, —S(O)₂OR, —NO₂, —S(O)₂N(R)₂, —N(R)S(O)₂R, —NH(CH₂)ₜ₁N(R)₂, —NH(CH₂)ₚ₁O(CH₂)_{q1}N(R)₂, —NH(CH₂)ₛ₁OR, —N((CH₂)ₛOR)₂, —N(R)-carbocycle, —N(R)-heterocycle, —N(R)-aryl, —N(R)-heteroaryl, —N(R)(CH₂)ₜ₁-carbocycle, —N(R)(CH₂)ₜ₁-heterocycle, —N(R)(CH₂)ₜ₁-aryl, —N(R)(CH₂)ₜ₁-heteroaryl, a carbocycle, a heterocycle, aryl and heteroaryl;
each R is independently selected from the group consisting of C₁₋₁₂ alkyl, C₂₋₁₂ alkenyl, and H;
m is selected from 5, 6, 7, 8, 9, 10, 11, 12, and 13;
n2 is selected from 1, 2, 3, 4, 5, 6, 7, 8, 9, and 10;
r is 0 or 1;
t¹ is selected from 1, 2, 3, 4, and 5;
p¹ is selected from 1, 2, 3, 4, and 5;
q¹ is selected from 1, 2, 3, 4, and 5; and
s¹ is selected from 1, 2, 3, 4.

In some aspects, the ionizable lipid is a compound of Formula (IL-IC):

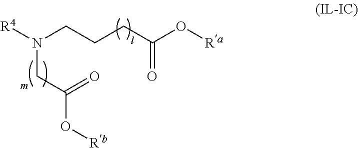

or its N-oxide, or a salt or isomer thereof,
wherein R′^{a} is R′^{branched} or R′^{cyclic}; wherein
R′^{branched} is

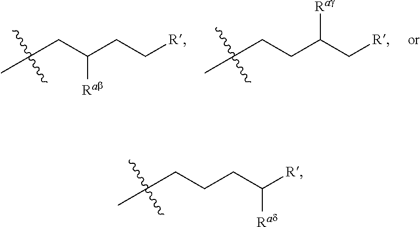

and R′^{cyclic} is:

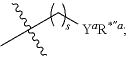

and
R′b is:

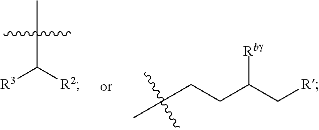

wherein

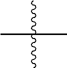

denotes a point of attachment;
wherein R^{aγ}, R^{aγ}, and R^{aγ} are each C₁₋₁₂ alkyl or C₂₋₁₂ alkenyl;
R^{bγ} is H, C₁₋₁₂ alkyl or C₂₋₁₂ alkenyl;
R² and R³ are each independently selected from the group consisting of C₁₋₁₄ alkyl and C₂₋₁₄ alkenyl;
R⁴ is —(CH₂)ₙOH; or

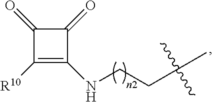

wherein

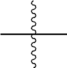

denotes a point of attachment;
each R′ independently is a C₁₋₁₂ alkyl or C₂₋₁₂ alkenyl;
R¹⁰ is N(R)₂; each R is independently selected from the group consisting of C₁₋₆ alkyl, C₂₋₃ alkenyl, and H; and n and n2 are each selected from the group consisting of 1, 2, 3, 4, and 5;
Y^{a} is a C₃₋₆ carbocycle;
R*″^{a} is selected from the group consisting of C₁₋₁₅ alkyl and C₂₋₁₅ alkenyl;
l is selected from 1, 2, 3, 4, and 5;
m is selected from 5, 6, 7, 8, and 9; and
s is 2 or 3.

In some embodiments, the ionizable lipid is a compound selected from Table IL-1.

TABLE IL-1
Ionizable lipids

| Cpd | |
|---|---|
| # | Structure |
| I-1 | 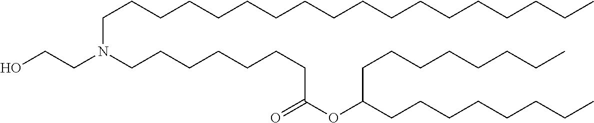 [SMILES: CCCCCCCCCCCCCCCCCCN(CCO)CCCCCCCC(=O)OC(CCCCCCCC)CCCCCCCC] |
| I-2 | 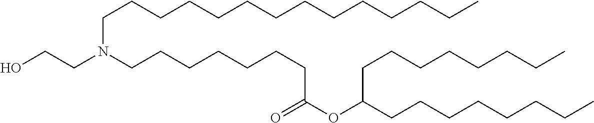 [SMILES: CCCCCCCCCCCCCCN(CCO)CCCCCCCC(=O)OC(CCCCCCCC)CCCCCCCC] |
| I-3 | 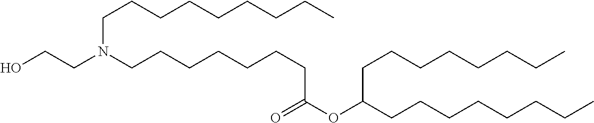 [SMILES: CCCCCCCCCN(CCO)CCCCCCCC(=O)OC(CCCCCCCC)CCCCCCCC] |
| I-4 | 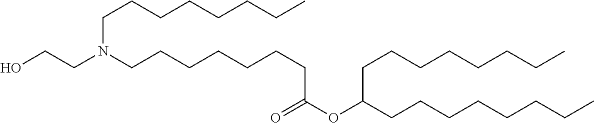 [SMILES: CCCCCCCCC(CCCCCCCC)OC(=O)CCCCCCCN(CCO)CCCCCCCC] |
| I-5 | 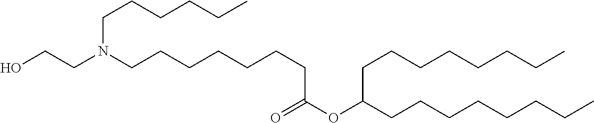 [SMILES: CCCCCCCCC(CCCCCCCC)OC(=O)CCCCCCCN(CCO)CCCCCC] |
| I-6 | 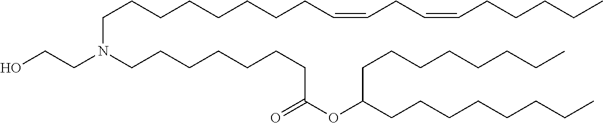 [SMILES: CCCCC/C=C\C/C=C\CCCCCCCCN(CCO)CCCCCCCC(=O)OC(CCCCCCCC)CCCCCCCC] |
| I-7 | 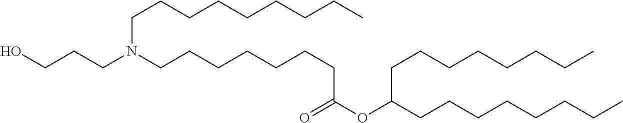 [SMILES: CCCCCCCCCN(CCCO)CCCCCCCC(=O)OC(CCCCCCCC)CCCCCCCC] |
| I-8 | 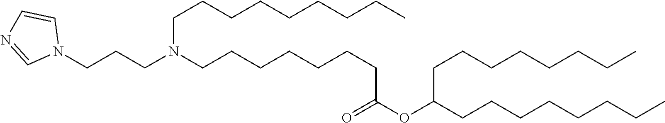 [SMILES: CCCCCCCCCN(CCCCCCCC(=O)OC(CCCCCCCC)CCCCCCCC)CCCn1ccnc1] |
| I-9 | 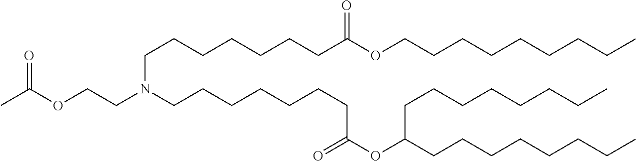 [SMILES: CCCCCCCCCOC(=O)CCCCCCCN(CCCCCCCC(=O)OC(CCCCCCCC)CCCCCCCC)CCOC(C)=O] |
| I-10 | 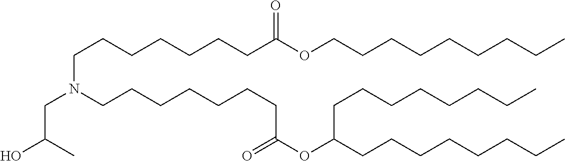 [SMILES: CCCCCCCCCOC(=O)CCCCCCCN(CCCCCCCC(=O)OC(CCCCCCCC)CCCCCCCC)CC(C)O] |
| I-11 | 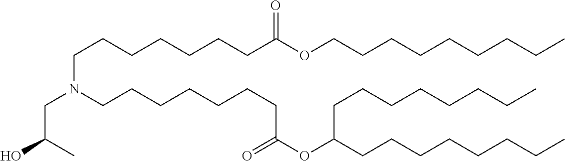 [SMILES: CCCCCCCCCOC(=O)CCCCCCCN(CCCCCCCC(=O)OC(CCCCCCCC)CCCCCCCC)C[C@@H](C)O] |
| I-12 | 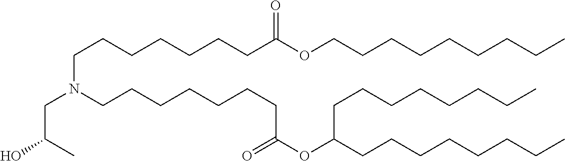 [SMILES: CCCCCCCCCOC(=O)CCCCCCCN(CCCCCCCC(=O)OC(CCCCCCCC)CCCCCCCC)C[C@H](C)O] |
| I-13 | 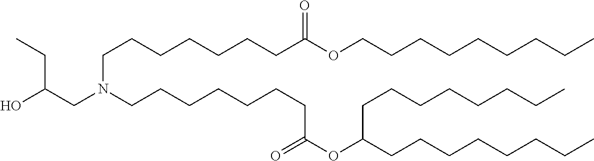 [SMILES: CCCCCCCCCOC(=O)CCCCCCCN(CCCCCCCC(=O)OC(CCCCCCCC)CCCCCCCC)CC(O)CC] |
| I-14 | 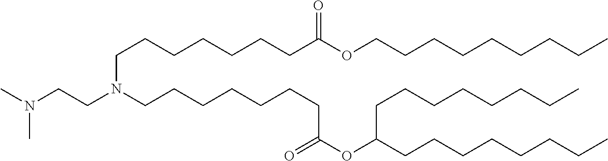 [SMILES: CCCCCCCCCOC(=O)CCCCCCCN(CCCCCCCC(=O)OC(CCCCCCCC)CCCCCCCC)CCN(C)C] |
| I-15 | 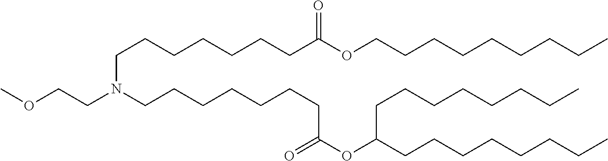 [SMILES: CCCCCCCCCOC(=O)CCCCCCCN(CCCCCCCC(=O)OC(CCCCCCCC)CCCCCCCC)CCOC] |
| I-16 | 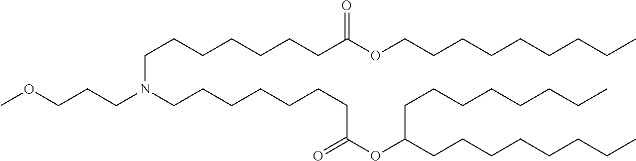 [SMILES: CCCCCCCCCOC(=O)CCCCCCCN(CCCCCCCC(=O)OC(CCCCCCCC)CCCCCCCC)CCCOC] |
| I-17 | 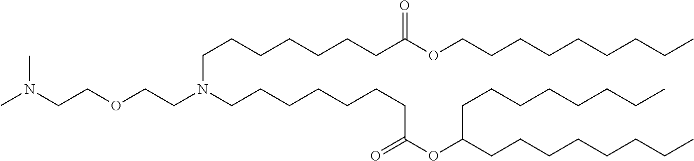 [SMILES: CCCCCCCCCOC(=O)CCCCCCCN(CCCCCCCC(=O)OC(CCCCCCCC)CCCCCCCC)CCOCCN(C)C] |
| I-18 | 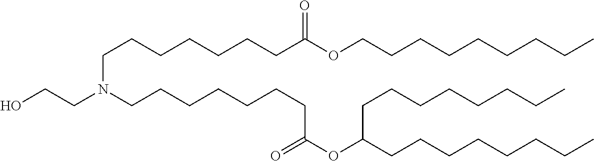 [SMILES: CCCCCCCCCOC(=O)CCCCCCCN(CCO)CCCCCCCC(=O)OC(CCCCCCCC)CCCCCCCC] |
| I-19 | 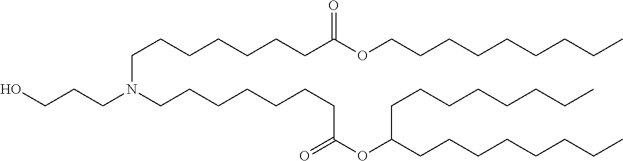 [SMILES: CCCCCCCCCOC(=O)CCCCCCCN(CCCO)CCCCCCCC(=O)OC(CCCCCCCC)CCCCCCCC] |
| I-20 | 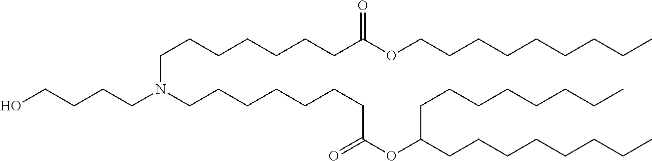 [SMILES: CCCCCCCCCOC(=O)CCCCCCCN(CCCCO)CCCCCCCC(=O)OC(CCCCCCCC)CCCCCCCC] |
| I-21 | 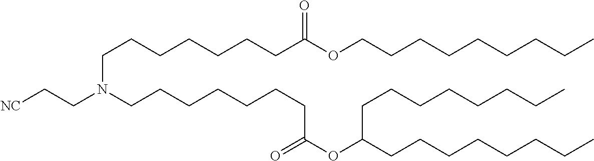 [SMILES: CCCCCCCCCOC(=O)CCCCCCCN(CCC#N)CCCCCCCC(=O)OC(CCCCCCCC)CCCCCCCC] |
| I-22 | 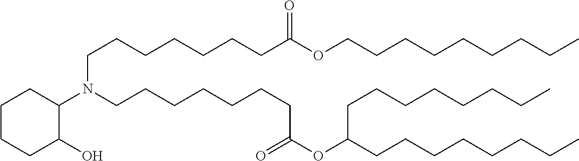 [SMILES: CCCCCCCCCOC(=O)CCCCCCCN(CCCCCCCC(=O)OC(CCCCCCCC)CCCCCCCC)C1CCCCC1O] |
| I-23 | 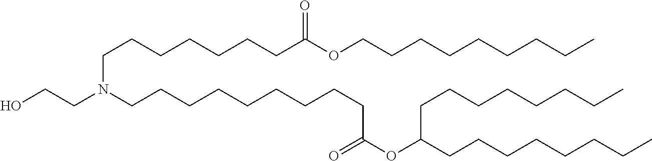 [SMILES: CCCCCCCCCOC(=O)CCCCCCCN(CCO)CCCCCCCCCC(=O)OC(CCCCCCCC)CCCCCCCC] |
| I-24 | 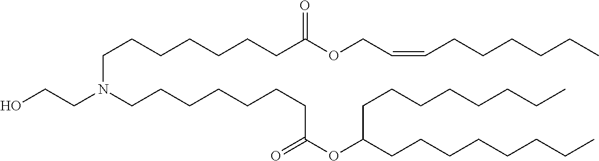 [SMILES: CCCCCC/C=C\COC(=O)CCCCCCCN(CCO)CCCCCCCC(=O)OC(CCCCCCCC)CCCCCCCC] |
| I-25 | 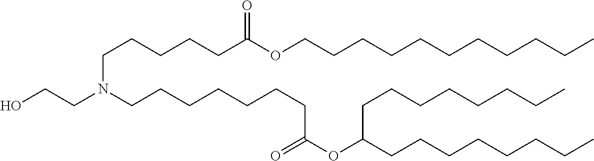 [SMILES: CCCCCCCCCCCOC(=O)CCCCCN(CCO)CCCCCCCC(=O)OC(CCCCCCCC)CCCCCCCC] |
| I-26 | 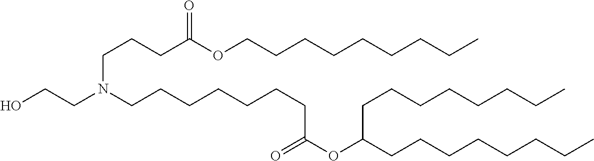 [SMILES: CCCCCCCCCOC(=O)CCCN(CCO)CCCCCCCC(=O)OC(CCCCCCCC)CCCCCCCC] |
| I-27 | 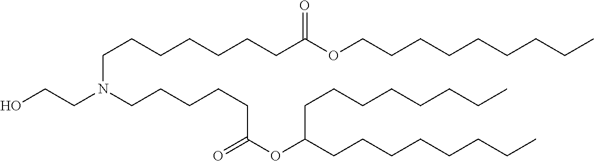 [SMILES: CCCCCCCCCOC(=O)CCCCCCCN(CCO)CCCCCC(=O)OC(CCCCCCCC)CCCCCCCC] |
| I-28 | 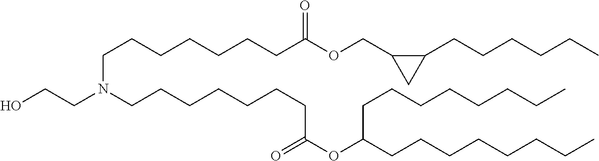 [SMILES: CCCCCCCCC(CCCCCCCC)OC(=O)CCCCCCCN(CCO)CCCCCCCC(=O)OCC1CC1CCCCCC] |
| I-29 | 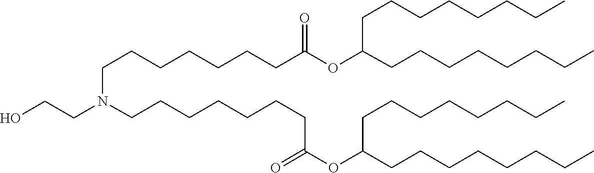 [SMILES: CCCCCCCCC(CCCCCCCC)OC(=O)CCCCCCCN(CCO)CCCCCCCC(=O)OC(CCCCCCCC)CCCCCCCC] |
| I-30 | 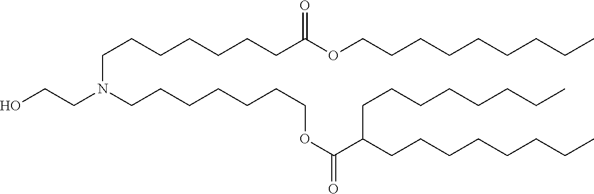 [SMILES: CCCCCCCCCOC(=O)CCCCCCCN(CCO)CCCCCCCOC(=O)C(CCCCCCCC)CCCCCCCC] |
| I-31 | 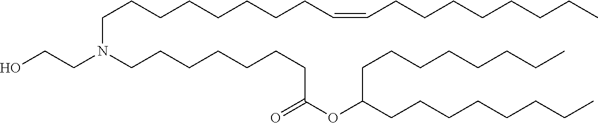 [SMILES: CCCCCCCC/C=C\CCCCCCCCN(CCO)CCCCCCCC(=O)OC(CCCCCCCC)CCCCCCCC] |
| I-32 | 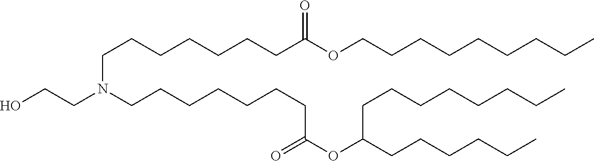 [SMILES: CCCCCCCCCOC(=O)CCCCCCCN(CCO)CCCCCCCC(=O)OC(CCCCCC)CCCCCCCC] |
| I-33 | 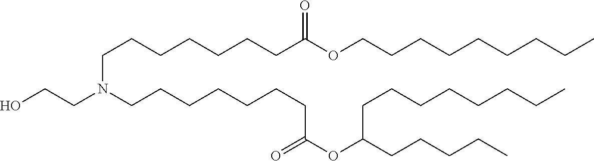 [SMILES: CCCCCCCCCOC(=O)CCCCCCCN(CCO)CCCCCCCC(=O)OC(CCCCC)CCCCCCCC] |
| I-34 | 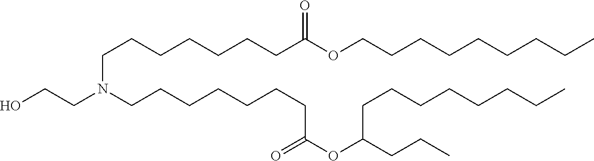 [SMILES: CCCCCCCCCOC(=O)CCCCCCCN(CCO)CCCCCCCC(=O)OC(CCC)CCCCCCCC] |
| I-35 | 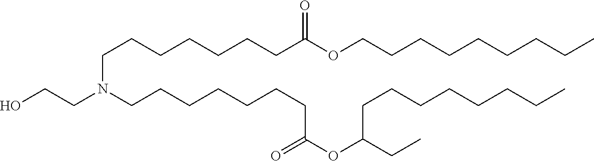 [SMILES: CCCCCCCCCOC(=O)CCCCCCCN(CCO)CCCCCCCC(=O)OC(CC)CCCCCCCC] |
| I-36 | 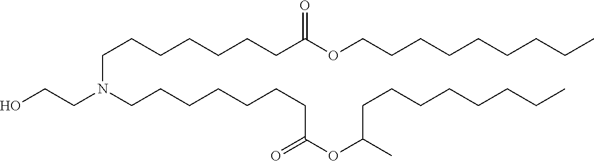 [SMILES: CCCCCCCCCOC(=O)CCCCCCCN(CCO)CCCCCCCC(=O)OC(C)CCCCCCCC] |
| I-37 | 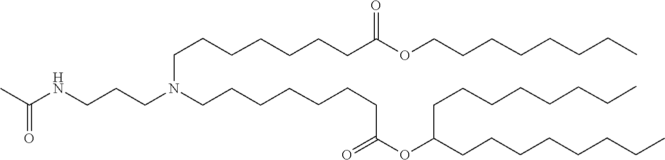 [SMILES: CCCCCCCCOC(=O)CCCCCCCN(CCCCCCCC(=O)OC(CCCCCCCC)CCCCCCCC)CCCNC(C)=O] |
| I-38 | 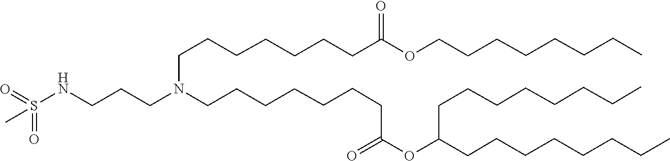 [SMILES: CCCCCCCCOC(=O)CCCCCCCN(CCCCCCCC(=O)OC(CCCCCCCC)CCCCCCCC)CCCNS(C)(=O)=O] |
| I-39 | 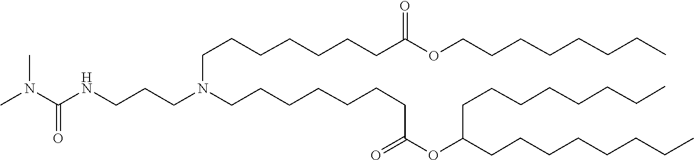 [SMILES: CCCCCCCCOC(=O)CCCCCCCN(CCCCCCCC(=O)OC(CCCCCCCC)CCCCCCCC)CCCNC(=O)N(C)C] |
| I-40 | 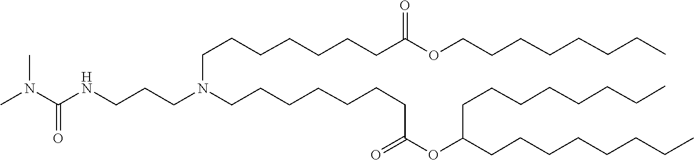 [SMILES: CCCCCCCCOC(=O)CCCCCCCN(CCCCCCCC(=O)OC(CCCCCCCC)CCCCCCCC)CCCNC(=O)N(C)C] |
| I-41 | 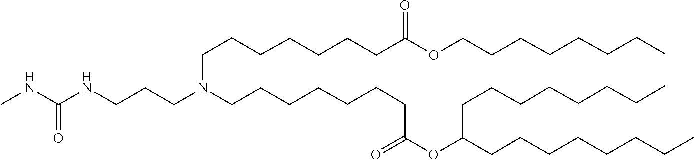 [SMILES: CCCCCCCCOC(=O)CCCCCCCN(CCCCCCCC(=O)OC(CCCCCCCC)CCCCCCCC)CCCNC(=O)NC] |
| I-42 | 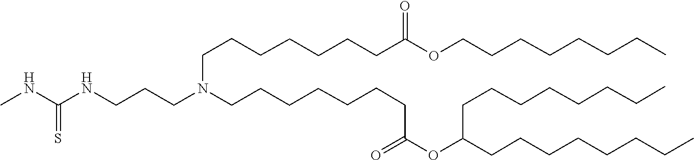 [SMILES: CCCCCCCCOC(=O)CCCCCCCN(CCCCCCCC(=O)OC(CCCCCCCC)CCCCCCCC)CCCNC(=S)NC] |
| I-43 | 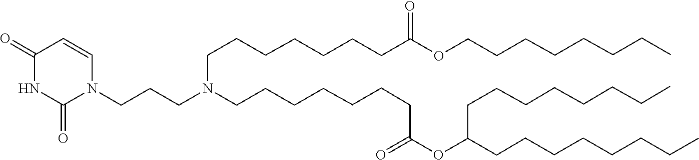 [SMILES: CCCCCCCCOC(=O)CCCCCCCN(CCCCCCCC(=O)OC(CCCCCCCC)CCCCCCCC)CCCn1ccc(=O)[nH]c1=O] |
| I-44 | 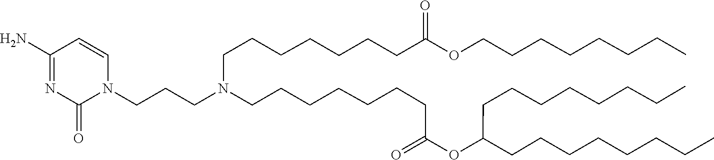 [SMILES: CCCCCCCCOC(=O)CCCCCCCN(CCCCCCCC(=O)OC(CCCCCCCC)CCCCCCCC)CCCn1ccc(N)nc1=O] |
| I-45 | 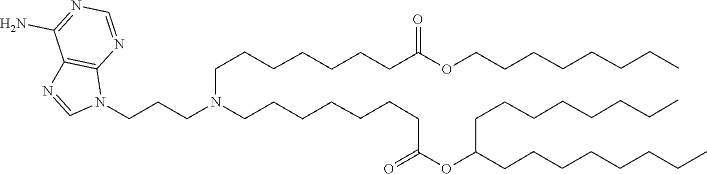 [SMILES: CCCCCCCCOC(=O)CCCCCCCN(CCCCCCCC(=O)OC(CCCCCCCC)CCCCCCCC)CCCn1cnc2c(N)ncnc21] |
| I-46 | 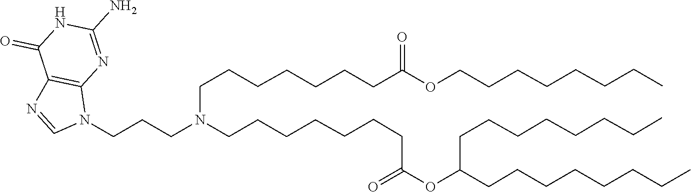 [SMILES: CCCCCCCCOC(=O)CCCCCCCN(CCCCCCCC(=O)OC(CCCCCCCC)CCCCCCCC)CCCn1cnc2c(=O)[nH]c(N)nc21] |
| I-47 | 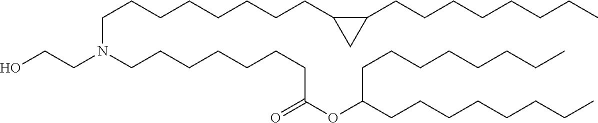 [SMILES: CCCCCCCCC(CCCCCCCC)OC(=O)CCCCCCCN(CCO)CCCCCCCCC1CC1CCCCCCCC] |
| I-48 | 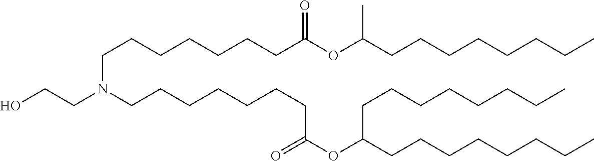 [SMILES: CCCCCCCCC(C)OC(=O)CCCCCCCN(CCO)CCCCCCCC(=O)OC(CCCCCCCC)CCCCCCCC] |
| I-49 | 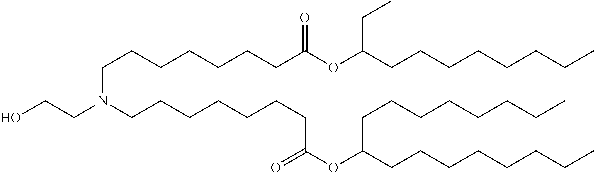 [SMILES: CCCCCCCCC(CC)OC(=O)CCCCCCCN(CCO)CCCCCCCC(=O)OC(CCCCCCCC)CCCCCCCC] |
| I-50 | 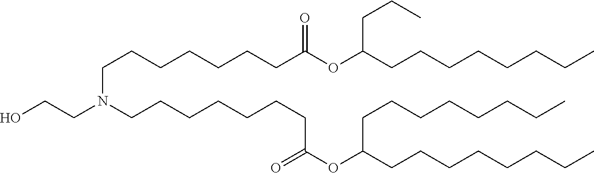 [SMILES: CCCCCCCCC(CCC)OC(=O)CCCCCCCN(CCO)CCCCCCCC(=O)OC(CCCCCCCC)CCCCCCCC] |
| I-51 | 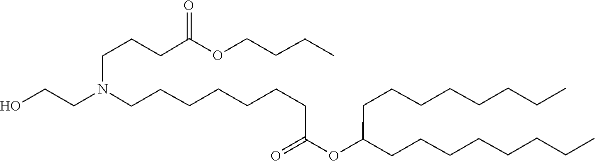 [SMILES: CCCCCCCCC(CCCCCCCC)OC(=O)CCCCCCCN(CCO)CCCC(=O)OCCCC] |
| I-52 | 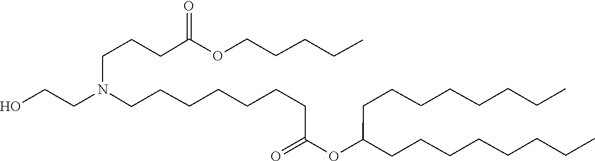 [SMILES: CCCCCCCCC(CCCCCCCC)OC(=O)CCCCCCCN(CCO)CCCC(=O)OCCCCC] |
| I-53 | 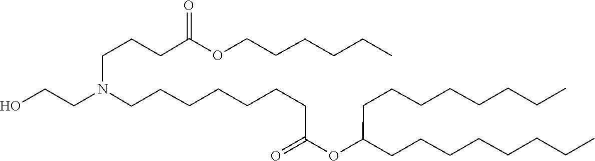 [SMILES: CCCCCCCCC(CCCCCCCC)OC(=O)CCCCCCCN(CCO)CCCC(=O)OCCCCCC] |
| I-54 | 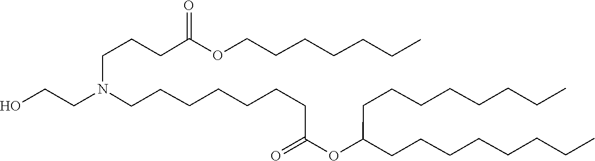 [SMILES: CCCCCCCCC(CCCCCCCC)OC(=O)CCCCCCCN(CCO)CCCC(=O)OCCCCCCC] |
| I-55 | 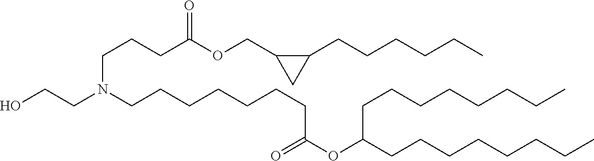 [SMILES: CCCCCCCCC(CCCCCCCC)OC(=O)CCCCCCCN(CCO)CCCC(=O)OCC1CC1CCCCCC] |
| I-56 | 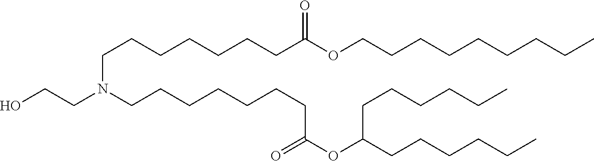 [SMILES: CCCCCCCCCOC(=O)CCCCCCCN(CCO)CCCCCCCC(=O)OC(CCCCCC)CCCCCC] |
| I-57 | 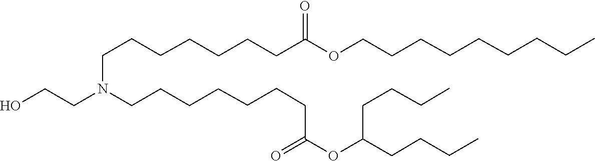 [SMILES: CCCCCCCCCOC(=O)CCCCCCCN(CCO)CCCCCCCC(=O)OC(CCCC)CCCC] |
| I-58 | 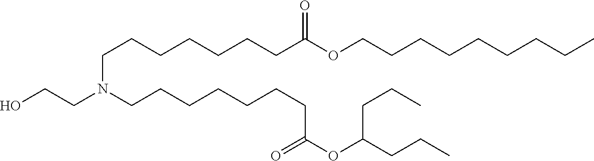 [SMILES: CCCCCCCCCOC(=O)CCCCCCCN(CCO)CCCCCCCC(=O)OC(CCC)CCC] |
| I-59 | 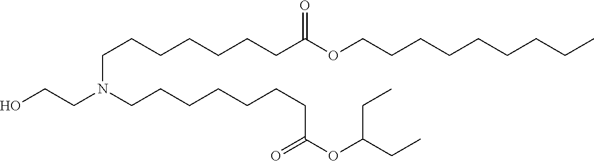 [SMILES: CCCCCCCCCOC(=O)CCCCCCCN(CCO)CCCCCCCC(=O)OC(CC)CC] |
| I-60 | 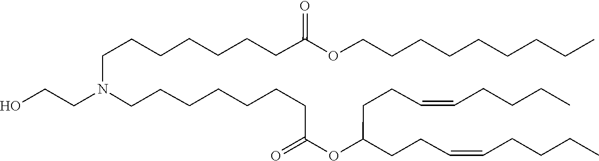 [SMILES: CCCC/C=C\CCC(CC/C=C\CCCC)OC(=O)CCCCCCCN(CCO)CCCCCCCC(=O)OCCCCCCCCC] |
| I-61 | 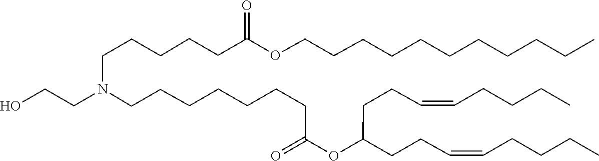 [SMILES: CCCC/C=C\CCC(CC/C=C\CCCC)OC(=O)CCCCCCCN(CCO)CCCCCC(=O)OCCCCCCCCCCC] |
| I-62 | 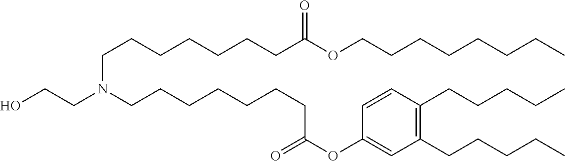 [SMILES: CCCCCCCCOC(=O)CCCCCCCN(CCO)CCCCCCCC(=O)Oc1ccc(CCCCC)c(CCCCC)c1] |
| I-63 | 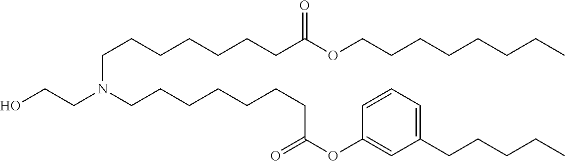 [SMILES: CCCCCCCCOC(=O)CCCCCCCN(CCO)CCCCCCCC(=O)Oc1cccc(CCCCC)c1] |
| I-64 | 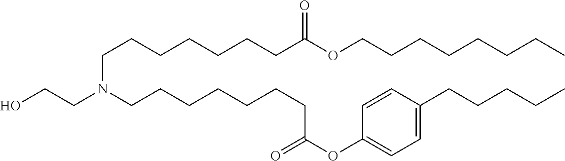 [SMILES: CCCCCCCCOC(=O)CCCCCCCN(CCO)CCCCCCCC(=O)Oc1ccc(CCCCC)cc1] |
| I-65 | 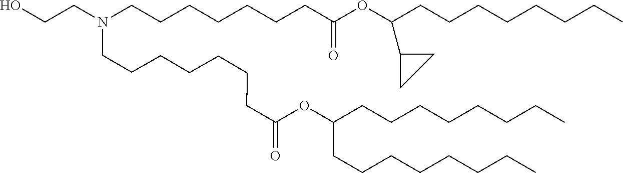 [SMILES: CCCCCCCCC(CCCCCCCC)OC(=O)CCCCCCCN(CCO)CCCCCCCC(=O)OC(CCCCCCCC)C1CC1] |
| I-66 | 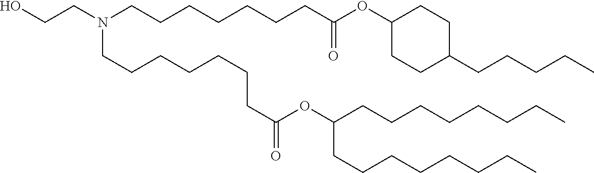 [SMILES: CCCCCCCCC(CCCCCCCC)OC(=O)CCCCCCCN(CCO)CCCCCCCC(=O)OC1CCC(CCCCC)CC1] |
| I-67 | 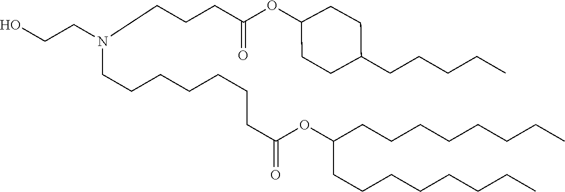 [SMILES: CCCCCCCCC(CCCCCCCC)OC(=O)CCCCCCCN(CCO)CCCC(=O)OC1CCC(CCCCC)CC1] |
| I-68 | 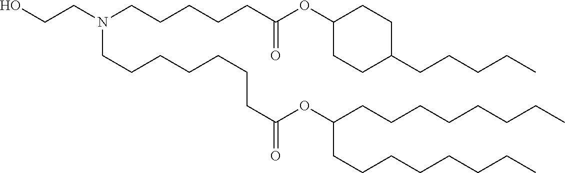 [SMILES: CCCCCCCCC(CCCCCCCC)OC(=O)CCCCCCCN(CCO)CCCCCC(=O)OC1CCC(CCCCC)CC1] |
| I-69 | 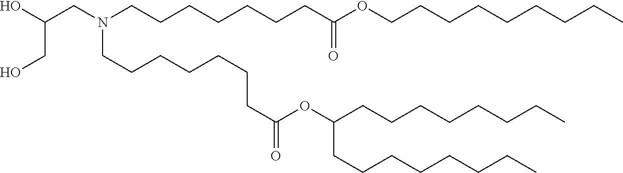 [SMILES: CCCCCCCCCOC(=O)CCCCCCCN(CCCCCCCC(=O)OC(CCCCCCCC)CCCCCCCC)CC(O)CO] |
| I-70 | 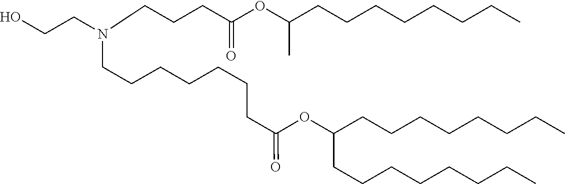 [SMILES: CCCCCCCCC(C)OC(=O)CCCN(CCO)CCCCCCCC(=O)OC(CCCCCCCC)CCCCCCCC] |
| I-71 | 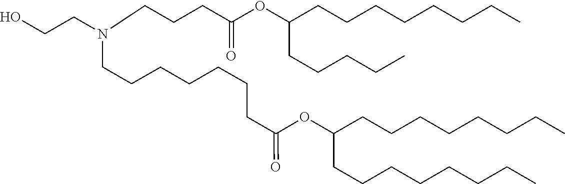 [SMILES: CCCCCCCCC(CCCCCCCC)OC(=O)CCCCCCCN(CCO)CCCC(=O)OC(CCCCC)CCCCCCCC] |
| I-72 | 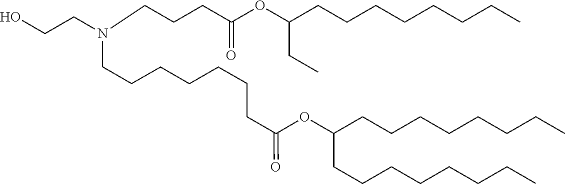 [SMILES: CCCCCCCCC(CC)OC(=O)CCCN(CCO)CCCCCCCC(=O)OC(CCCCCCCC)CCCCCCCC] |
| I-73 | 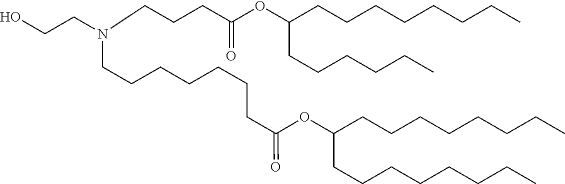 [SMILES: CCCCCCCCC(CCCCCCCC)OC(=O)CCCCCCCN(CCO)CCCC(=O)OC(CCCCCC)CCCCCCCC] |
| I-74 | 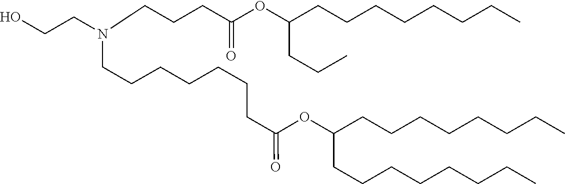 [SMILES: CCCCCCCCC(CCC)OC(=O)CCCN(CCO)CCCCCCCC(=O)OC(CCCCCCCC)CCCCCCCC] |
| I-75 | 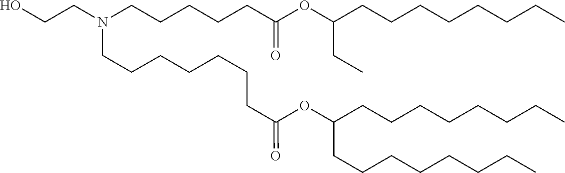 [SMILES: CCCCCCCCC(CC)OC(=O)CCCCCN(CCO)CCCCCCCC(=O)OC(CCCCCCCC)CCCCCCCC] |
| I-76 | 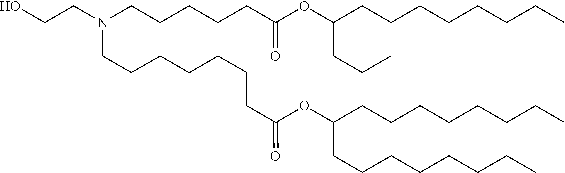 [SMILES: CCCCCCCCC(CCC)OC(=O)CCCCCN(CCO)CCCCCCCC(=O)OC(CCCCCCCC)CCCCCCCC] |
| I-77 | 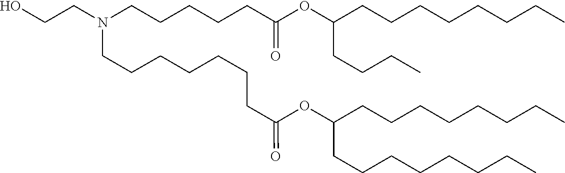 [SMILES: CCCCCCCCC(CCCC)OC(=O)CCCCCN(CCO)CCCCCCCC(=O)OC(CCCCCCCC)CCCCCCCC] |
| I-78 | 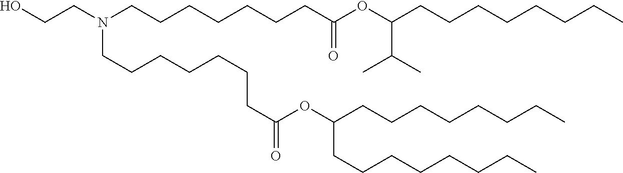 [SMILES: CCCCCCCCC(CCCCCCCC)OC(=O)CCCCCCCN(CCO)CCCCCCCC(=O)OC(CCCCCCCC)C(C)C] |
| I-79 | 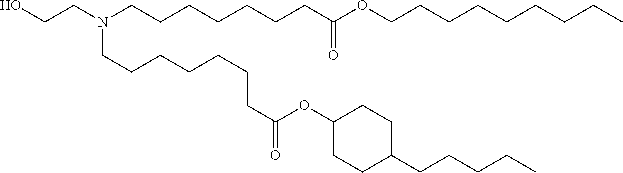 [SMILES: CCCCCCCCCOC(=O)CCCCCCCN(CCO)CCCCCCCC(=O)OC1CCC(CCCCC)CC1] |
| I-80 | 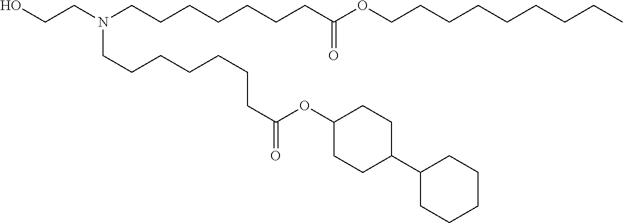 [SMILES: CCCCCCCCCOC(=O)CCCCCCCN(CCO)CCCCCCCC(=O)OC1CCC(C2CCCCC2)CC1] |
| I-81 | 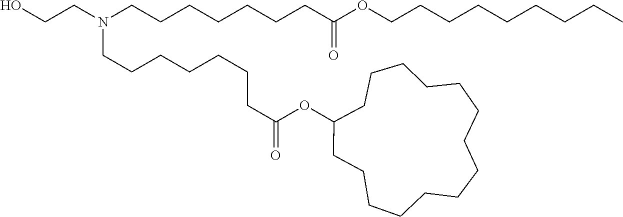 [SMILES: CCCCCCCCCOC(=O)CCCCCCCN(CCO)CCCCCCCC(=O)OC1CCCCCCCCCCCCCC1] |
| I-82 | 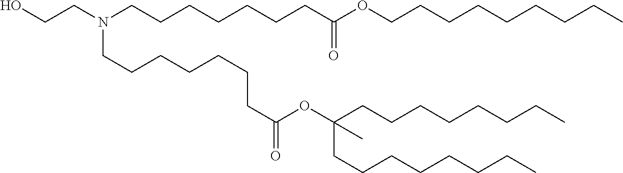 [SMILES: CCCCCCCCCOC(=O)CCCCCCCN(CCO)CCCCCCCC(=O)OC(C)(CCCCCCCC)CCCCCCCC] |
| I-83 | 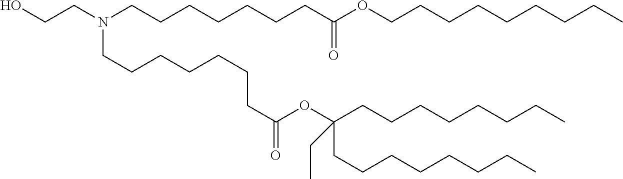 [SMILES: CCCCCCCCCOC(=O)CCCCCCCN(CCO)CCCCCCCC(=O)OC(CC)(CCCCCCCC)CCCCCCCC] |
| I-84 | 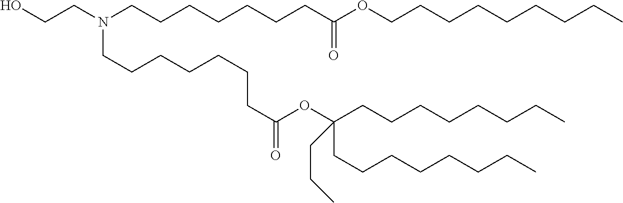 [SMILES: CCCCCCCCCOC(=O)CCCCCCCN(CCO)CCCCCCCC(=O)OC(CCC)(CCCCCCCC)CCCCCCCC] |
| I-85 | 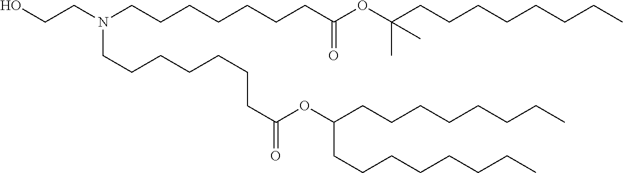 [SMILES: CCCCCCCCC(CCCCCCCC)OC(=O)CCCCCCCN(CCO)CCCCCCCC(=O)OC(C)(C)CCCCCCCC] |
| I-86 | 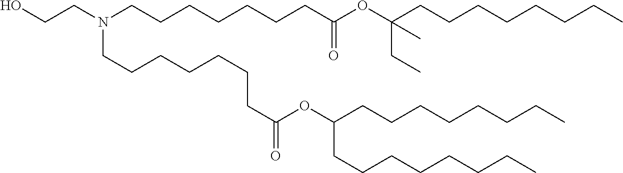 [SMILES: CCCCCCCCC(CCCCCCCC)OC(=O)CCCCCCCN(CCO)CCCCCCCC(=O)OC(C)(CC)CCCCCCCC] |
| I-87 | 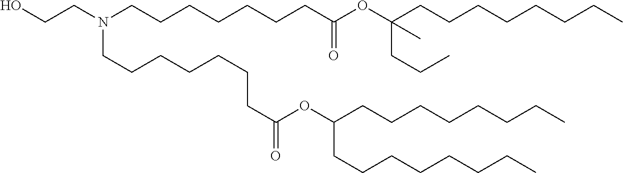 [SMILES: CCCCCCCCC(CCCCCCCC)OC(=O)CCCCCCCN(CCO)CCCCCCCC(=O)OC(C)(CCC)CCCCCCCC] |
| I-88 | 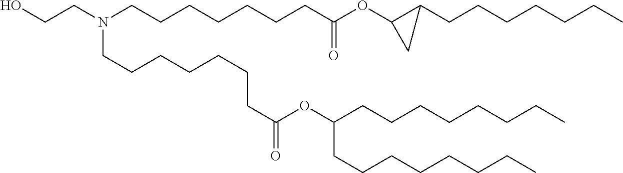 [SMILES: CCCCCCCCC(CCCCCCCC)OC(=O)CCCCCCCN(CCO)CCCCCCCC(=O)OC1CC1CCCCCCC] |
| I-89 | 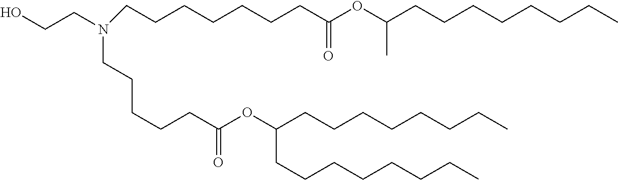 [SMILES: CCCCCCCCC(C)OC(=O)CCCCCCCN(CCO)CCCCCC(=O)OC(CCCCCCCC)CCCCCCCC] |
| I-90 | 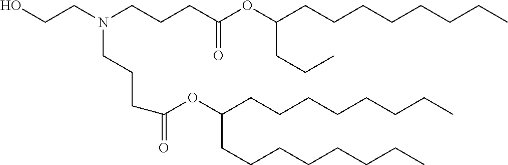 [SMILES: CCCCCCCCC(CCC)OC(=O)CCCN(CCO)CCCC(=O)OC(CCCCCCCC)CCCCCCCC] |
| I-91 | 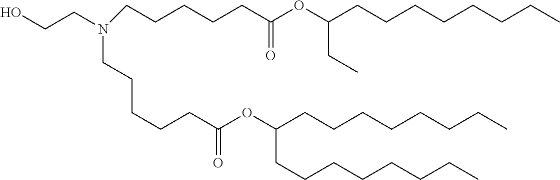 [SMILES: CCCCCCCCC(CC)OC(=O)CCCCCN(CCO)CCCCCC(=O)OC(CCCCCCCC)CCCCCCCC] |
| I-92 | 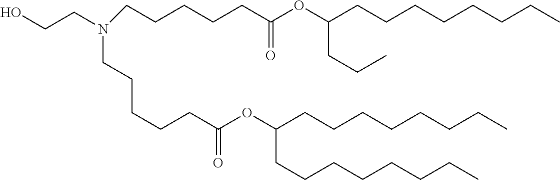 [SMILES: CCCCCCCCC(CCC)OC(=O)CCCCCN(CCO)CCCCCC(=O)OC(CCCCCCCC)CCCCCCCC] |
| I-93 | 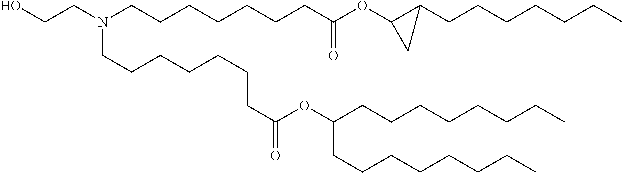 [SMILES: CCCCCCCCC(CCCCCCCC)OC(=O)CCCCCCCN(CCO)CCCCCCCC(=O)OC1CC1CCCCCCC] |
| I-94 | 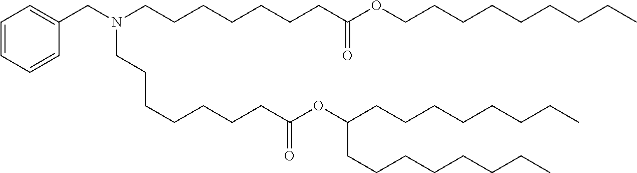 [SMILES: CCCCCCCCCOC(=O)CCCCCCCN(CCCCCCCC(=O)OC(CCCCCCCC)CCCCCCCC)Cc1ccccc1] |
| I-95 | 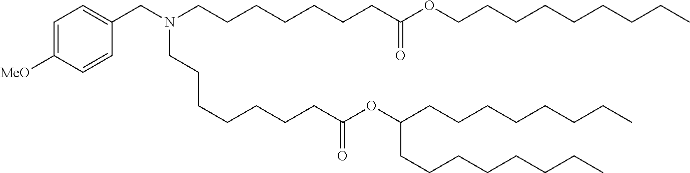 [SMILES: CCCCCCCCCOC(=O)CCCCCCCN(CCCCCCCC(=O)OC(CCCCCCCC)CCCCCCCC)Cc1ccc(OC)cc1] |
| I-96 | 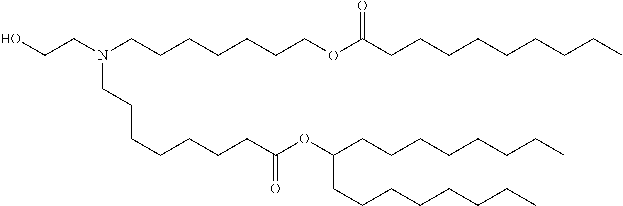 [SMILES: CCCCCCCCCC(=O)OCCCCCCCN(CCO)CCCCCCCC(=O)OC(CCCCCCCC)CCCCCCCC] |
| I-97 | 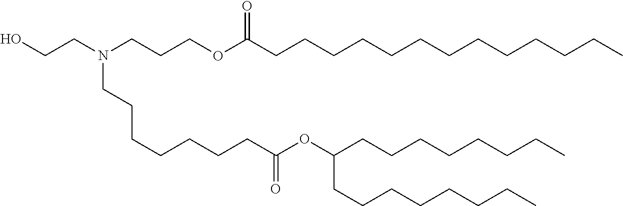 [SMILES: CCCCCCCCCCCCCC(=O)OCCCN(CCO)CCCCCCCC(=O)OC(CCCCCCCC)CCCCCCCC] |
| I-98 | 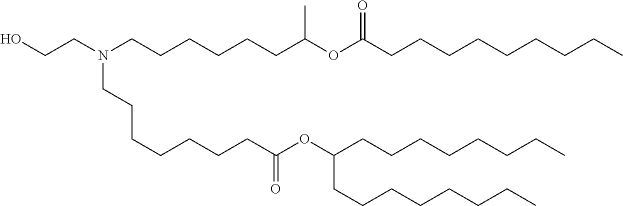 [SMILES: CCCCCCCCCC(=O)OC(C)CCCCCCN(CCO)CCCCCCCC(=O)OC(CCCCCCCC)CCCCCCCC] |
| I-99 | 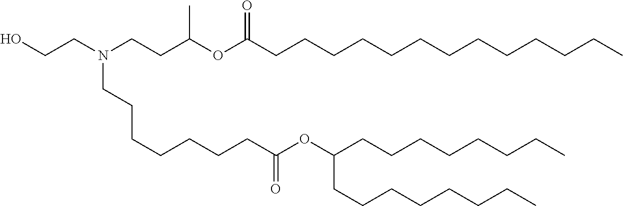 [SMILES: CCCCCCCCCCCCCC(=O)OC(C)CCN(CCO)CCCCCCCC(=O)OC(CCCCCCCC)CCCCCCCC] |
| I-100 | 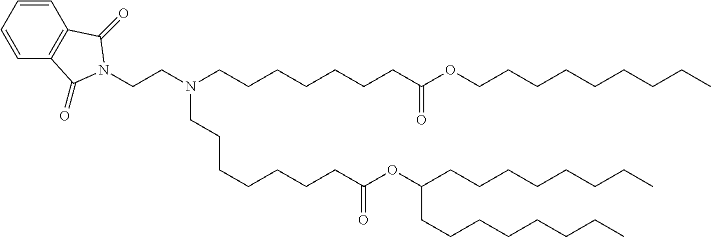 [SMILES: CCCCCCCCCOC(=O)CCCCCCCN(CCCCCCCC(=O)OC(CCCCCCCC)CCCCCCCC)CCN1C(=O)c2ccccc2C1=O] |
| I-101 | 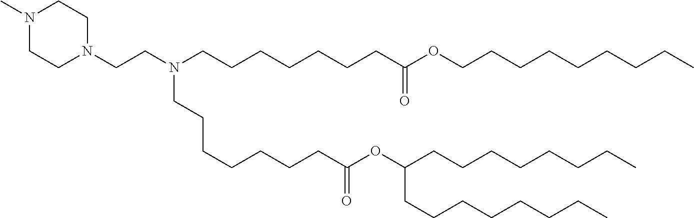 [SMILES: CCCCCCCCCOC(=O)CCCCCCCN(CCCCCCCC(=O)OC(CCCCCCCC)CCCCCCCC)CCN1CCN(C)CC1] |
| I-102 | 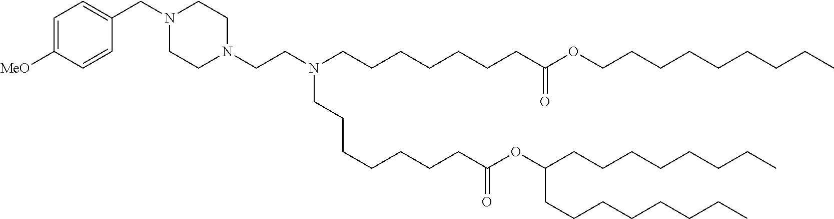 [SMILES: CCCCCCCCCOC(=O)CCCCCCCN(CCCCCCCC(=O)OC(CCCCCCCC)CCCCCCCC)CCN1CCN(Cc2ccc(OC)cc2)CC1] |
| I-103 | 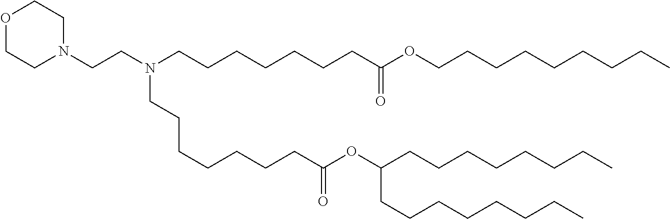 [SMILES: CCCCCCCCCOC(=O)CCCCCCCN(CCCCCCCC(=O)OC(CCCCCCCC)CCCCCCCC)CCN1CCOCC1] |
| I-104 | 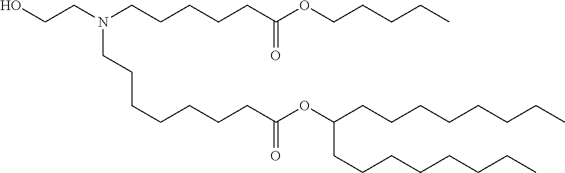 [SMILES: CCCCCCCCC(CCCCCCCC)OC(=O)CCCCCCCN(CCO)CCCCCC(=O)OCCCCC] |
| I-105 | 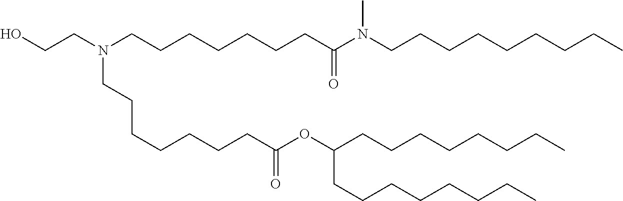 [SMILES: CCCCCCCCCN(C)C(=O)CCCCCCCN(CCO)CCCCCCCC(=O)OC(CCCCCCCC)CCCCCCCC] |
| I-106 | 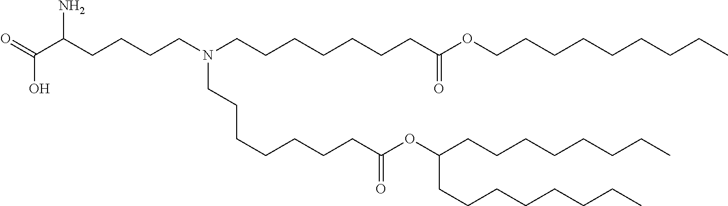 [SMILES: CCCCCCCCCOC(=O)CCCCCCCN(CCCCCCCC(=O)OC(CCCCCCCC)CCCCCCCC)CCCCC(N)C(=O)O] |
| I-107 | 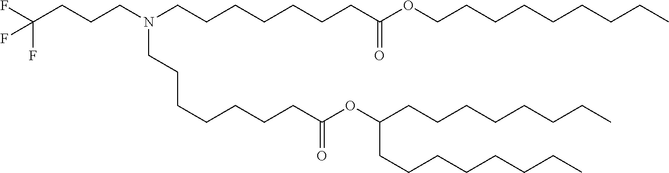 [SMILES: CCCCCCCCCOC(=O)CCCCCCCN(CCCCCCCC(=O)OC(CCCCCCCC)CCCCCCCC)CCCC(F)(F)F] |
| I-108 | 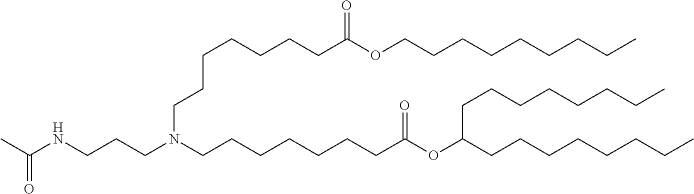 [SMILES: CCCCCCCCCOC(=O)CCCCCCCN(CCCCCCCC(=O)OC(CCCCCCCC)CCCCCCCC)CCCNC(C)=O] |
| I-109 | 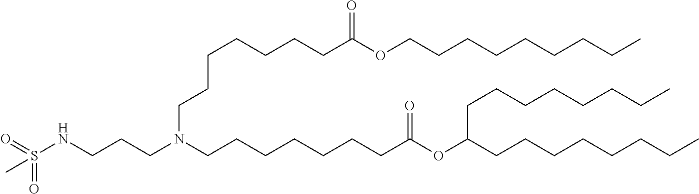 [SMILES: CCCCCCCCCOC(=O)CCCCCCCN(CCCCCCCC(=O)OC(CCCCCCCC)CCCCCCCC)CCCNS(C)(=O)=O] |
| I-110 | 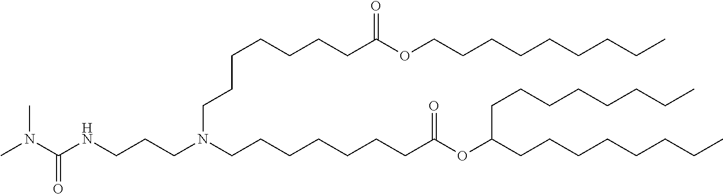 [SMILES: CCCCCCCCCOC(=O)CCCCCCCN(CCCCCCCC(=O)OC(CCCCCCCC)CCCCCCCC)CCCNC(=O)N(C)C] |
| I-111 | 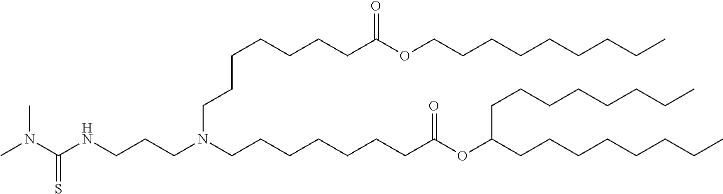 [SMILES: CCCCCCCCCOC(=O)CCCCCCCN(CCCCCCCC(=O)OC(CCCCCCCC)CCCCCCCC)CCCNC(=S)N(C)C] |
| I-112 | 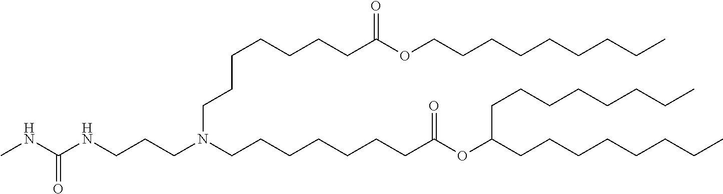 [SMILES: CCCCCCCCCOC(=O)CCCCCCCN(CCCCCCCC(=O)OC(CCCCCCCC)CCCCCCCC)CCCNC(=O)NC] |
| I-113 | 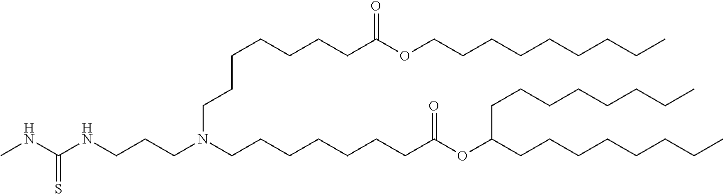 [SMILES: CCCCCCCCCOC(=O)CCCCCCCN(CCCCCCCC(=O)OC(CCCCCCCC)CCCCCCCC)CCCNC(=S)NC] |
| I-114 | 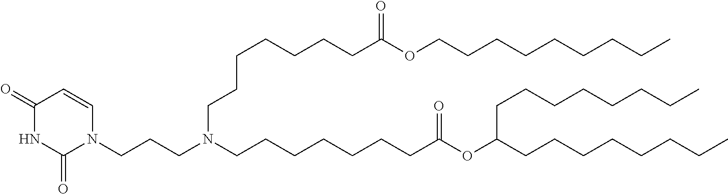 [SMILES: CCCCCCCCCOC(=O)CCCCCCCN(CCCCCCCC(=O)OC(CCCCCCCC)CCCCCCCC)CCCn1ccc(=O)[nH]c1=O] |
| I-115 | 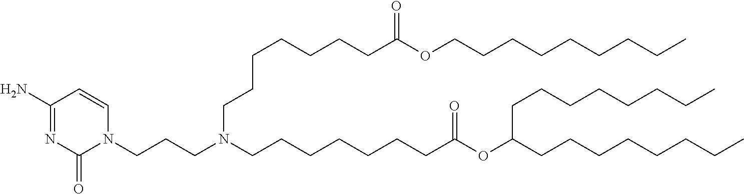 [SMILES: CCCCCCCCCOC(=O)CCCCCCCN(CCCCCCCC(=O)OC(CCCCCCCC)CCCCCCCC)CCCn1ccc(N)nc1=O] |
| I-116 | 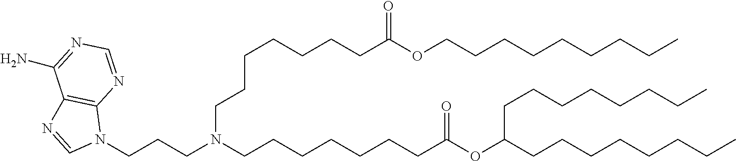 [SMILES: CCCCCCCCCOC(=O)CCCCCCCN(CCCCCCCC(=O)OC(CCCCCCCC)CCCCCCCC)CCCn1cnc2c(N)ncnc21] |
| I-117 | 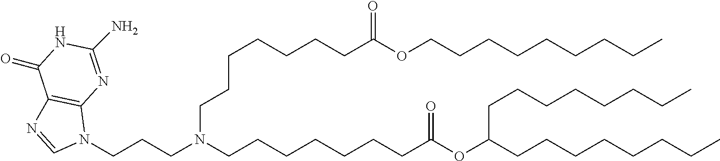 [SMILES: CCCCCCCCCOC(=O)CCCCCCCN(CCCCCCCC(=O)OC(CCCCCCCC)CCCCCCCC)CCCn1cnc2c(=O)[nH]c(N)nc21] |
| I-118 | 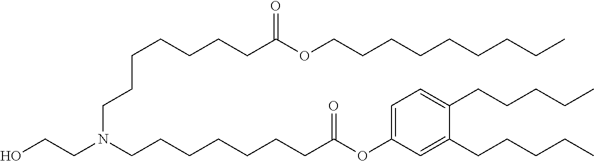 [SMILES: CCCCCCCCCOC(=O)CCCCCCCN(CCO)CCCCCCCC(=O)Oc1ccc(CCCCC)c(CCCCC)c1] |
| I-119 | 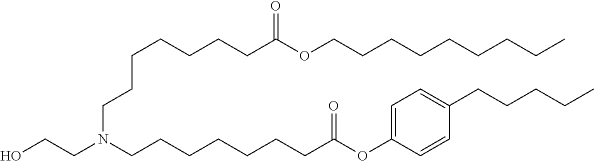 [SMILES: CCCCCCCCCOC(=O)CCCCCCCN(CCO)CCCCCCCC(=O)Oc1ccc(CCCCC)cc1] |
| I-120 | 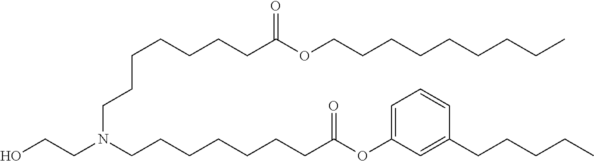 [SMILES: CCCCCCCCCOC(=O)CCCCCCCN(CCO)CCCCCCCC(=O)Oc1cccc(CCCCC)c1] |
| I-121 | 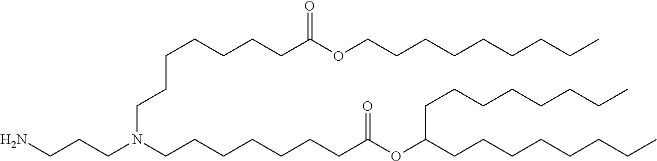 [SMILES: CCCCCCCCCOC(=O)CCCCCCCN(CCCN)CCCCCCCC(=O)OC(CCCCCCCC)CCCCCCCC] |
| I-122 | 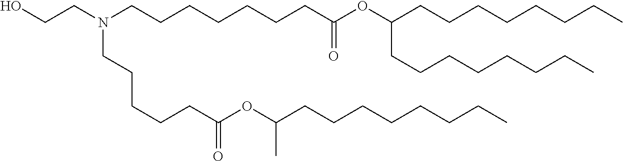 [SMILES: CCCCCCCCC(C)OC(=O)CCCCCN(CCO)CCCCCCCC(=O)OC(CCCCCCCC)CCCCCCCC] |
| I-123 | 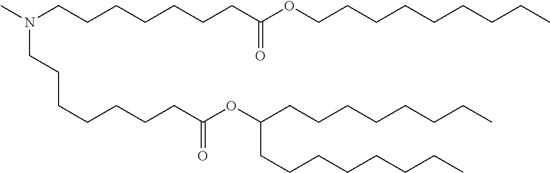 [SMILES: CCCCCCCCCOC(=O)CCCCCCCN(C)CCCCCCCC(=O)OC(CCCCCCCC)CCCCCCCC] |
| I-124 | 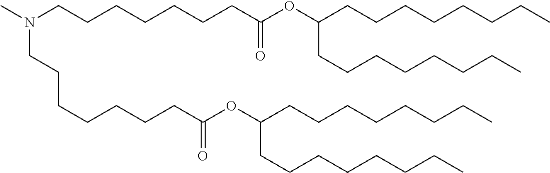 [SMILES: CCCCCCCCC(CCCCCCCC)OC(=O)CCCCCCCN(C)CCCCCCCC(=O)OC(CCCCCCCC)CCCCCCCC] |
| I-125 | 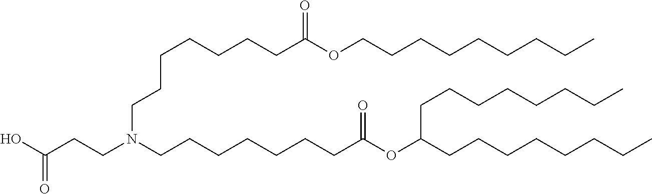 [SMILES: CCCCCCCCCOC(=O)CCCCCCCN(CCCCCCCC(=O)OC(CCCCCCCC)CCCCCCCC)CCC(=O)O] |
| I-126 | 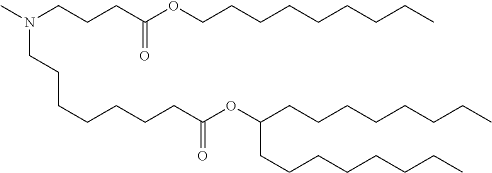 [SMILES: CCCCCCCCCOC(=O)CCCN(C)CCCCCCCC(=O)OC(CCCCCCCC)CCCCCCCC] |
| I-127 | 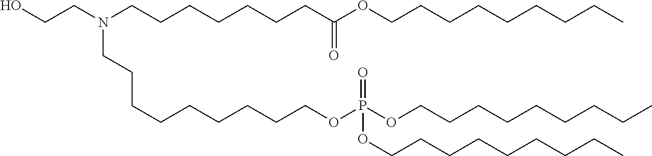 [SMILES: CCCCCCCCCOC(=O)CCCCCCCN(CCO)CCCCCCCCCOP(=O)(OCCCCCCCCC)OCCCCCCCCC] |
| I-128 | 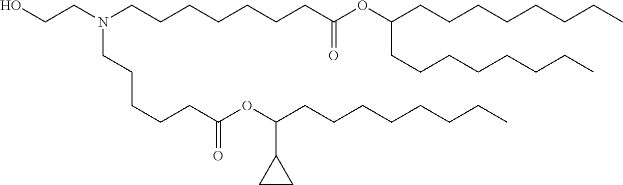 [SMILES: CCCCCCCCC(CCCCCCCC)OC(=O)CCCCCCCN(CCO)CCCCCC(=O)OC(CCCCCCCC)C1CC1] |
| I-129 | 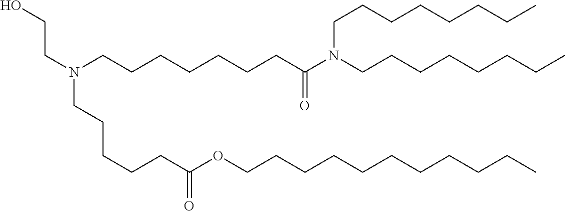 [SMILES: CCCCCCCCCCCOC(=O)CCCCCN(CCO)CCCCCCCC(=O)N(CCCCCCCC)CCCCCCCC] |
| I-130 | 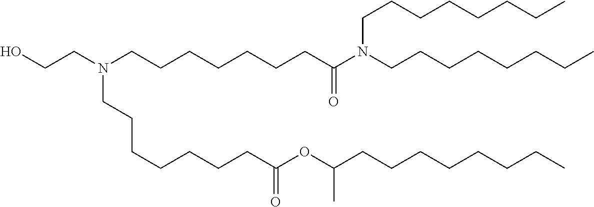 [SMILES: CCCCCCCCC(C)OC(=O)CCCCCCCN(CCO)CCCCCCCC(=O)N(CCCCCCCC)CCCCCCCC] |
| I-131 | 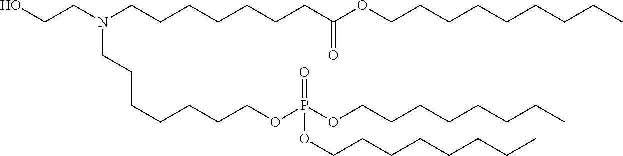 [SMILES: CCCCCCCCCOC(=O)CCCCCCCN(CCO)CCCCCCCOP(=O)(OCCCCCCCC)OCCCCCCCC] |
| I-132 | 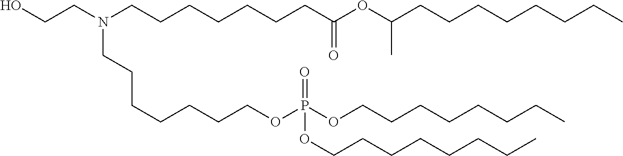 [SMILES: CCCCCCCCOP(=O)(OCCCCCCCC)OCCCCCCCN(CCO)CCCCCCCC(=O)OC(C)CCCCCCCC] |
| I-133 | 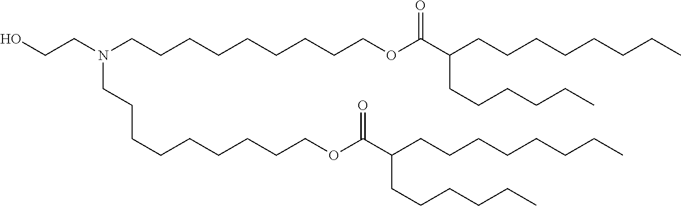 [SMILES: CCCCCCCCC(CCCCCC)C(=O)OCCCCCCCCCN(CCO)CCCCCCCCCOC(=O)C(CCCCCC)CCCCCCCC] |
| I-134 | 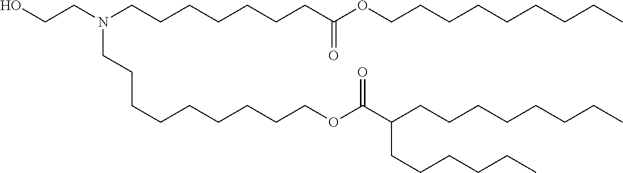 [SMILES: CCCCCCCCCOC(=O)CCCCCCCN(CCO)CCCCCCCCCOC(=O)C(CCCCCC)CCCCCCCC] |
| I-135 | 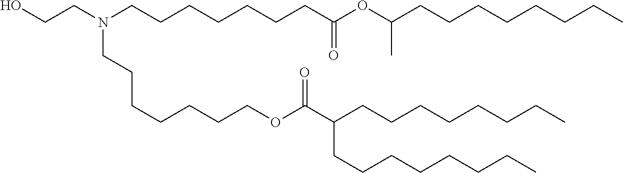 [SMILES: CCCCCCCCC(C)OC(=O)CCCCCCCN(CCO)CCCCCCCOC(=O)C(CCCCCCCC)CCCCCCCC] |
| I-136 | 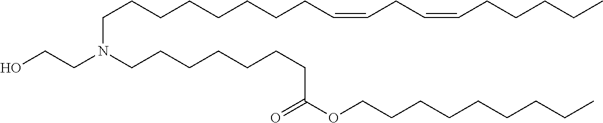 [SMILES: CCCCC/C=C\C/C=C\CCCCCCCCN(CCO)CCCCCCCC(=O)OCCCCCCCCC] |
| I-137 | 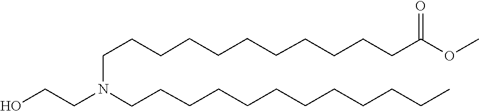 [SMILES: CCCCCCCCCCCCN(CCO)CCCCCCCCCCCC(=O)OC] |
| I-138 | 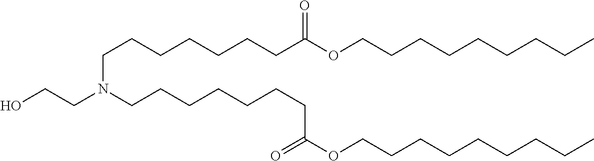 [SMILES: CCCCCCCCCOC(=O)CCCCCCCN(CCO)CCCCCCCC(=O)OCCCCCCCCC] |
| I-139 | 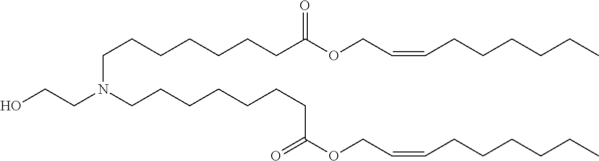 [SMILES: CCCCCC/C=C\COC(=O)CCCCCCCN(CCO)CCCCCCCC(=O)OC/C=C\CCCCCC] |
| I-140 | 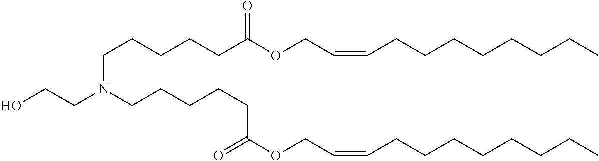 [SMILES: CCCCCCCC/C=C\COC(=O)CCCCCN(CCO)CCCCCC(=O)OC/C=C\CCCCCCCC] |
| I-141 | 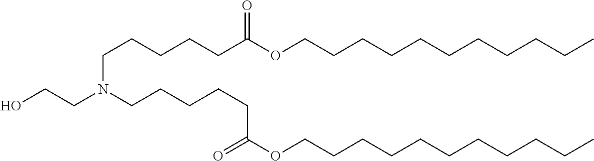 [SMILES: CCCCCCCCCCCOC(=O)CCCCCN(CCO)CCCCCC(=O)OCCCCCCCCCCC] |
| I-142 | 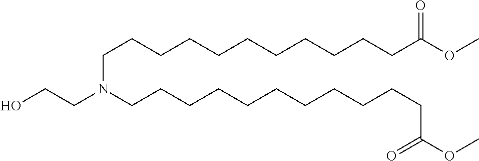 [SMILES: COC(=O)CCCCCCCCCCCN(CCO)CCCCCCCCCCCC(=O)OC] |
| I-143 | 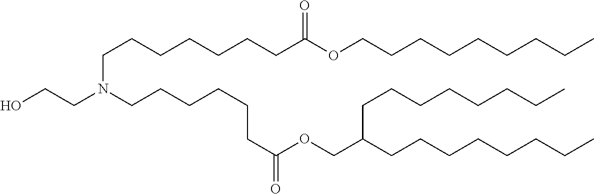 [SMILES: CCCCCCCCCOC(=O)CCCCCCCN(CCO)CCCCCCC(=O)OCC(CCCCCCCC)CCCCCCCC] |
| I-144 | 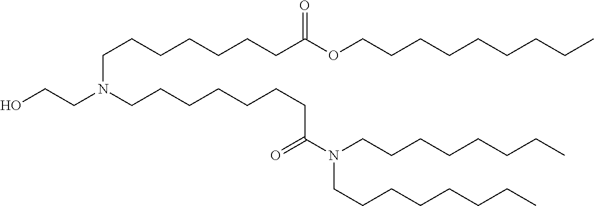 [SMILES: CCCCCCCCCOC(=O)CCCCCCCN(CCO)CCCCCCCC(=O)N(CCCCCCCC)CCCCCCCC] |
| I-145 | 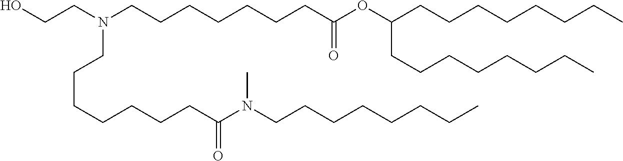 [SMILES: CCCCCCCCC(CCCCCCCC)OC(=O)CCCCCCCN(CCO)CCCCCCCC(=O)N(C)CCCCCCCC] |
| I-146 | 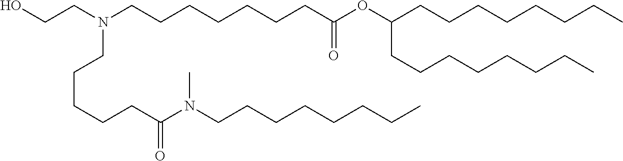 [SMILES: CCCCCCCCC(CCCCCCCC)OC(=O)CCCCCCCN(CCO)CCCCCC(=O)N(C)CCCCCCCC] |
| I-147 | 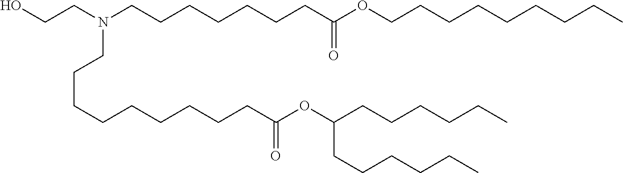 [SMILES: CCCCCCCCCOC(=O)CCCCCCCN(CCO)CCCCCCCCCC(=O)OC(CCCCCC)CCCCCC] |
| I-148 | 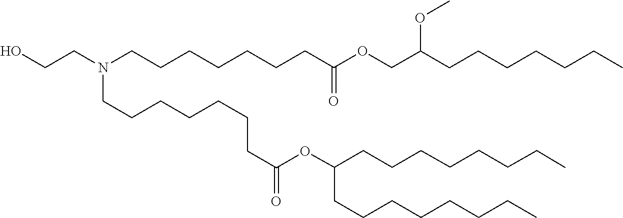 [SMILES: CCCCCCCCC(CCCCCCCC)OC(=O)CCCCCCCN(CCO)CCCCCCCC(=O)OCC(CCCCCCC)OC] |
| I-149 | 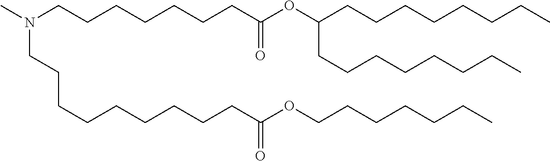 [SMILES: CCCCCCCCC(CCCCCCCC)OC(=O)CCCCCCCN(C)CCCCCCCCCC(=O)OCCCCCCC] |
| I-150 | 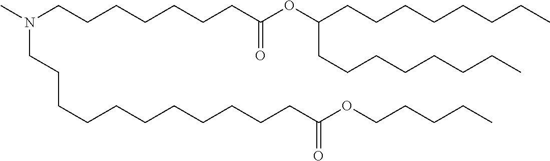 [SMILES: CCCCCCCCC(CCCCCCCC)OC(=O)CCCCCCCN(C)CCCCCCCCCCCC(=O)OCCCCC] |
| I-151 | 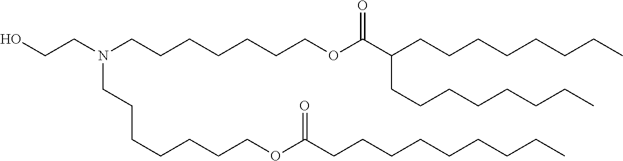 [SMILES: CCCCCCCCCC(=O)OCCCCCCCN(CCO)CCCCCCCOC(=O)C(CCCCCCCC)CCCCCCCC] |
| I-152 | 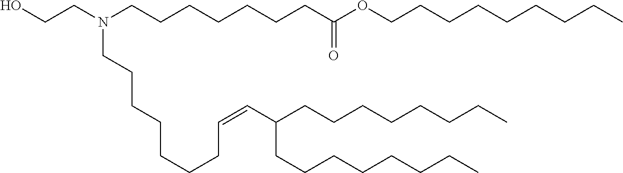 [SMILES: CCCCCCCCCOC(=O)CCCCCCCN(CCO)CCCCCCC/C=C\C(CCCCCCCC)CCCCCCCC] |
| I-153 | 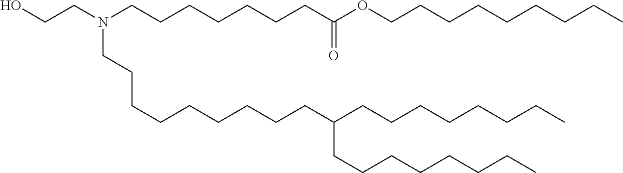 [SMILES: CCCCCCCCCOC(=O)CCCCCCCN(CCO)CCCCCCCCCC(CCCCCCCC)CCCCCCCC] |
| I-154 | 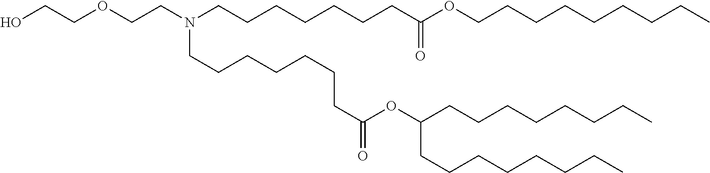 [SMILES: CCCCCCCCCOC(=O)CCCCCCCN(CCCCCCCC(=O)OC(CCCCCCCC)CCCCCCCC)CCOCCO] |
| I-155 | 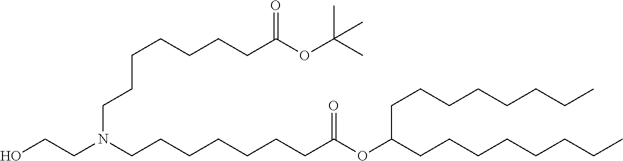 [SMILES: CCCCCCCCC(CCCCCCCC)OC(=O)CCCCCCCN(CCO)CCCCCCCC(=O)OC(C)(C)C] |
| I-156 | 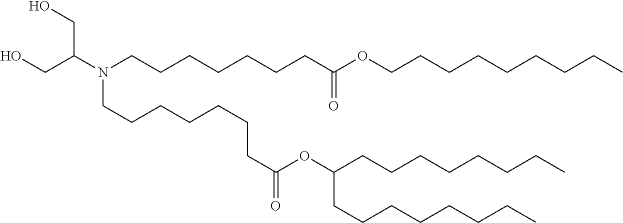 [SMILES: CCCCCCCCCOC(=O)CCCCCCCN(CCCCCCCC(=O)OC(CCCCCCCC)CCCCCCCC)C(CO)CO] |
| I-157 | 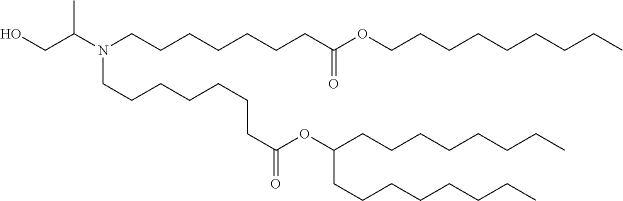 [SMILES: CCCCCCCCCOC(=O)CCCCCCCN(CCCCCCCC(=O)OC(CCCCCCCC)CCCCCCCC)C(C)CO] |
| I-158 | 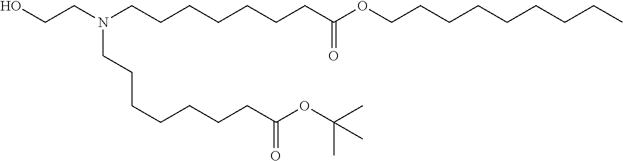 [SMILES: CCCCCCCCCOC(=O)CCCCCCCN(CCO)CCCCCCCC(=O)OC(C)(C)C] |
| I-159 | 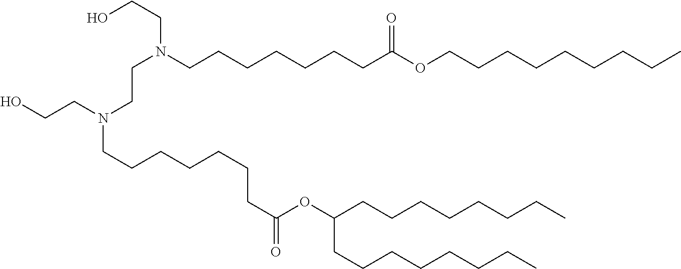 [SMILES: CCCCCCCCCOC(=O)CCCCCCCN(CCO)CCN(CCO)CCCCCCCC(=O)OC(CCCCCCCC)CCCCCCCC] |
| I-160 | 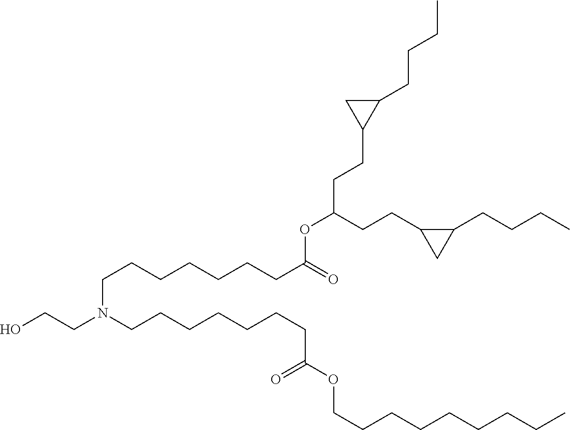 [SMILES: CCCCCCCCCOC(=O)CCCCCCCN(CCO)CCCCCCCC(=O)OC(CCC1CC1CCCC)CCC1CC1CCCC] |
| I-161 | 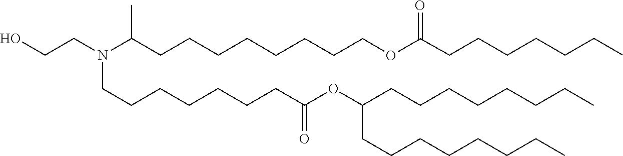 [SMILES: CCCCCCCCC(CCCCCCCC)OC(=O)CCCCCCCN(CCO)C(C)CCCCCCCCOC(=O)CCCCCCC] |
| I-162 | 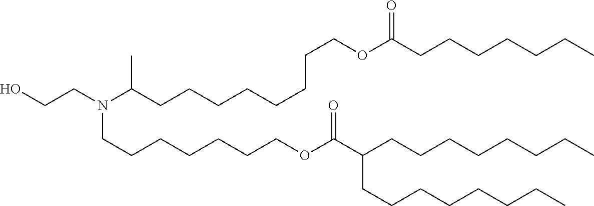 [SMILES: CCCCCCCCC(CCCCCCCC)C(=O)OCCCCCCCN(CCO)C(C)CCCCCCCCOC(=O)CCCCCCC] |
| I-163 | 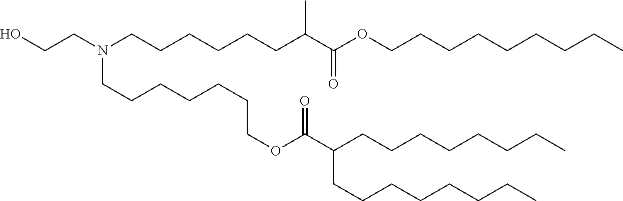 [SMILES: CCCCCCCCCOC(=O)C(C)CCCCCCN(CCO)CCCCCCCOC(=O)C(CCCCCCCC)CCCCCCCC] |
| I-164 | 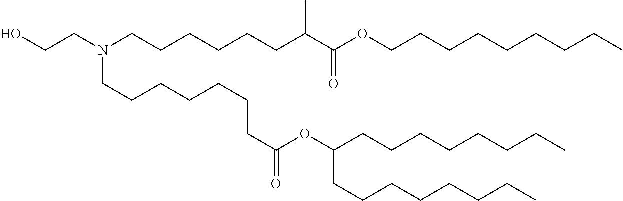 [SMILES: CCCCCCCCCOC(=O)C(C)CCCCCCN(CCO)CCCCCCCC(=O)OC(CCCCCCCC)CCCCCCCC] |
| I-165 | 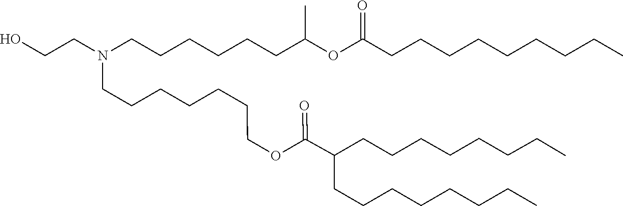 [SMILES: CCCCCCCCCC(=O)OC(C)CCCCCCN(CCO)CCCCCCCOC(=O)C(CCCCCCCC)CCCCCCCC] |
| I-166 | 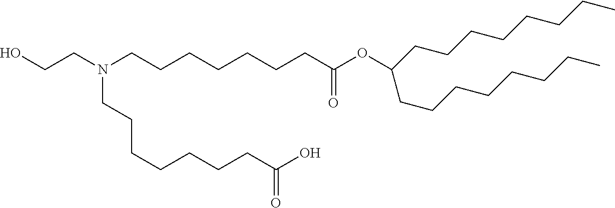 [SMILES: CCCCCCCCC(CCCCCCCC)OC(=O)CCCCCCCN(CCO)CCCCCCCC(=O)O] |
| I-167 | 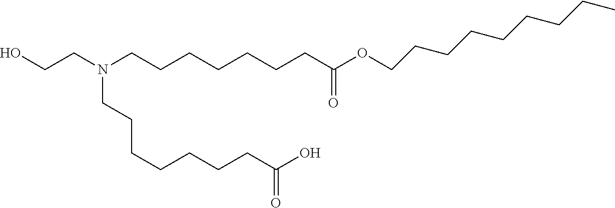 [SMILES: CCCCCCCCCOC(=O)CCCCCCCN(CCO)CCCCCCCC(=O)O] |
| I-168 | 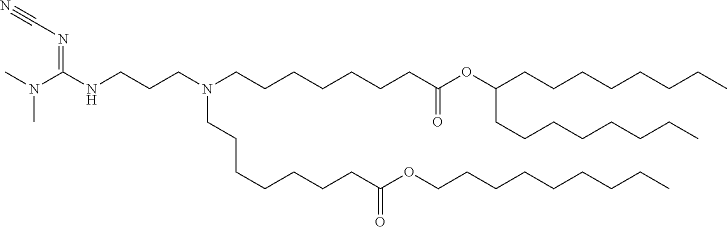 [SMILES: CCCCCCCCCOC(=O)CCCCCCCN(CCCCCCCC(=O)OC(CCCCCCCC)CCCCCCCC)CCCN/C(=N/C#N)N(C)C] |
| I-169 | 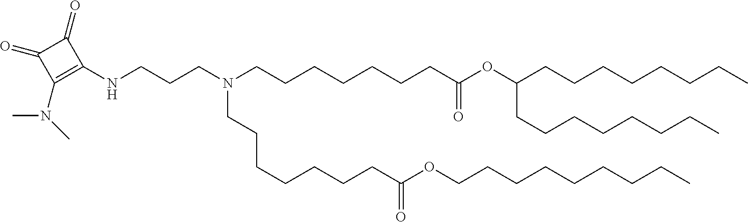 [SMILES: CCCCCCCCCOC(=O)CCCCCCCN(CCCCCCCC(=O)OC(CCCCCCCC)CCCCCCCC)CCCNc1c(N(C)C)c(=O)c1=O] |
| I-170 | 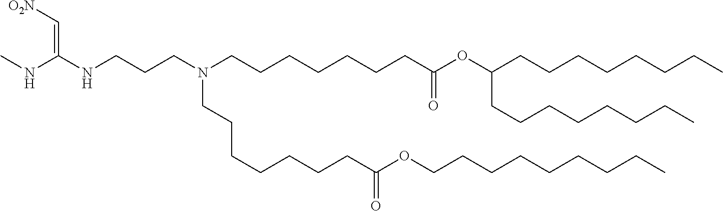 [SMILES: CCCCCCCCCOC(=O)CCCCCCCN(CCCCCCCC(=O)OC(CCCCCCCC)CCCCCCCC)CCCN/C(=C/[N+](=O)[O-])NC] |
| I-171 | 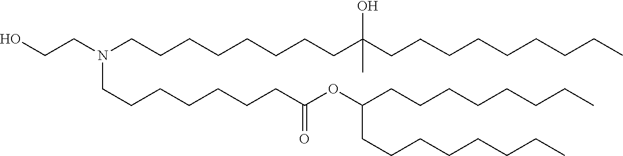 [SMILES: CCCCCCCCCC(C)(O)CCCCCCCCN(CCO)CCCCCCCC(=O)OC(CCCCCCCC)CCCCCCCC] |
| I-172 | 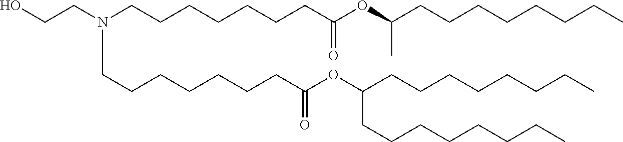 [SMILES: CCCCCCCCC(CCCCCCCC)OC(=O)CCCCCCCN(CCO)CCCCCCCC(=O)O[C@H](C)CCCCCCCC] |
| I-173 | 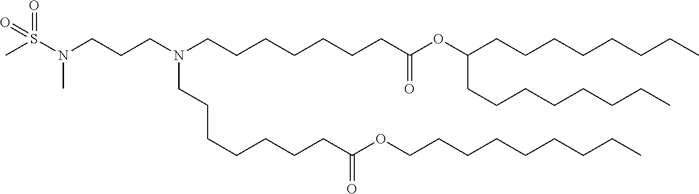 [SMILES: CCCCCCCCCOC(=O)CCCCCCCN(CCCCCCCC(=O)OC(CCCCCCCC)CCCCCCCC)CCCN(C)S(C)(=O)=O] |
| I-174 | 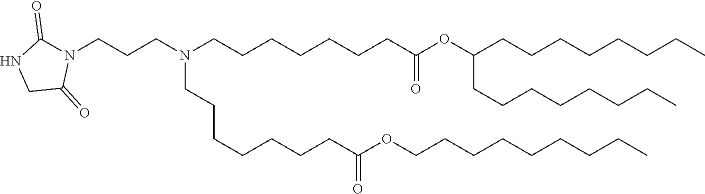 [SMILES: CCCCCCCCCOC(=O)CCCCCCCN(CCCCCCCC(=O)OC(CCCCCCCC)CCCCCCCC)CCCN1C(=O)CNC1=O] |
| I-175 | 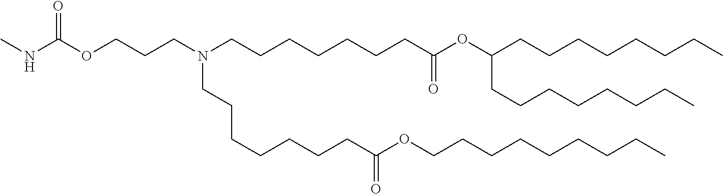 [SMILES: CCCCCCCCCOC(=O)CCCCCCCN(CCCCCCCC(=O)OC(CCCCCCCC)CCCCCCCC)CCCOC(=O)NC] |
| I-176 | 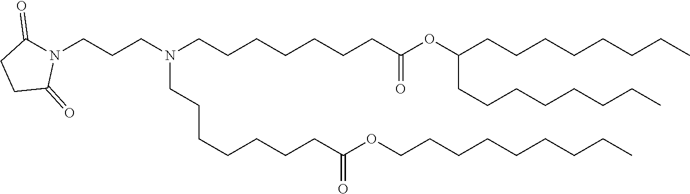 [SMILES: CCCCCCCCCOC(=O)CCCCCCCN(CCCCCCCC(=O)OC(CCCCCCCC)CCCCCCCC)CCCN1C(=O)CCC1=O] |
| I-177 | 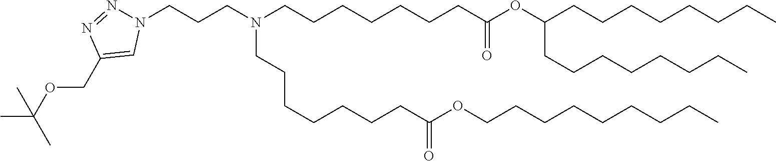 [SMILES: CCCCCCCCCOC(=O)CCCCCCCN(CCCCCCCC(=O)OC(CCCCCCCC)CCCCCCCC)CCCn1cc(COC(C)(C)C)nn1] |
| I-178 | 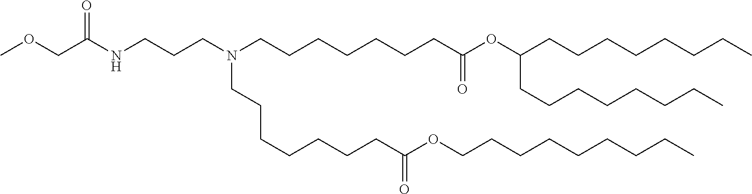 [SMILES: CCCCCCCCCOC(=O)CCCCCCCN(CCCCCCCC(=O)OC(CCCCCCCC)CCCCCCCC)CCCNC(=O)COC] |
| I-179 | 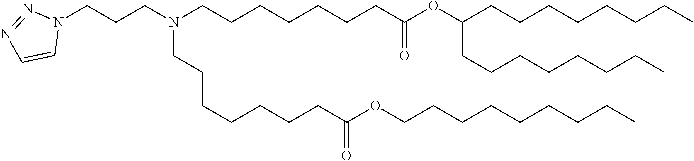 [SMILES: CCCCCCCCCOC(=O)CCCCCCCN(CCCCCCCC(=O)OC(CCCCCCCC)CCCCCCCC)CCCn1ccnn1] |
| I-180 | 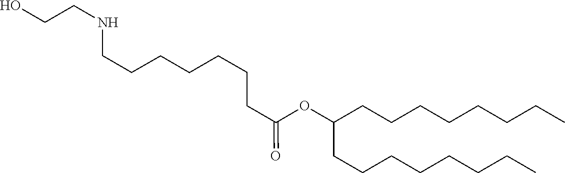 [SMILES: CCCCCCCCC(CCCCCCCC)OC(=O)CCCCCCCNCCO] |
| I-181 | 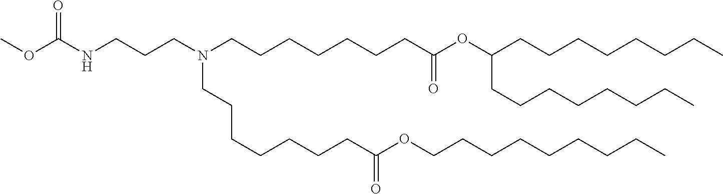 [SMILES: CCCCCCCCCOC(=O)CCCCCCCN(CCCCCCCC(=O)OC(CCCCCCCC)CCCCCCCC)CCCNC(=O)OC] |
| I-182 | 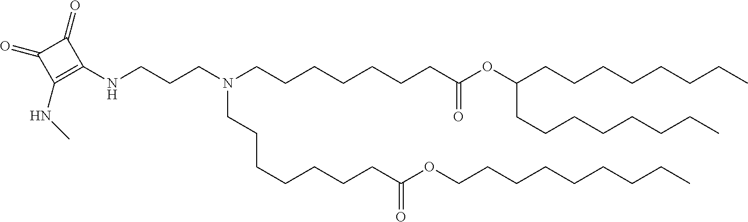 [SMILES: CCCCCCCCCOC(=O)CCCCCCCN(CCCCCCCC(=O)OC(CCCCCCCC)CCCCCCCC)CCCNc1c(NC)c(=O)c1=O] |
| I-183 | 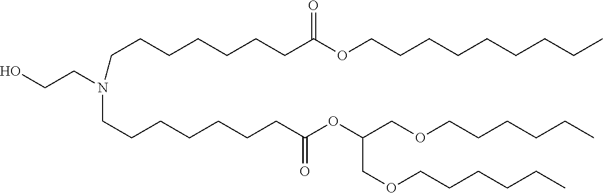 [SMILES: CCCCCCCCCOC(=O)CCCCCCCN(CCO)CCCCCCCC(=O)OC(COCCCCCC)COCCCCCC] |
| I-184 | 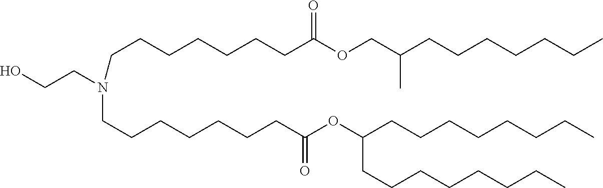 [SMILES: CCCCCCCCC(CCCCCCCC)OC(=O)CCCCCCCN(CCO)CCCCCCCC(=O)OCC(C)CCCCCCC] |
| I-185 | 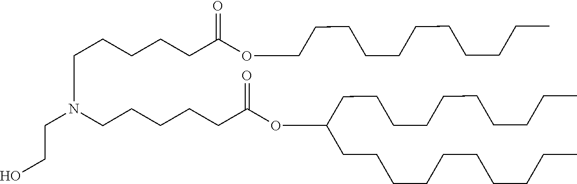 [SMILES: CCCCCCCCCCCOC(=O)CCCCCN(CCO)CCCCCC(=O)OC(CCCCCCCCCC)CCCCCCCCCC] |
| I-186 | 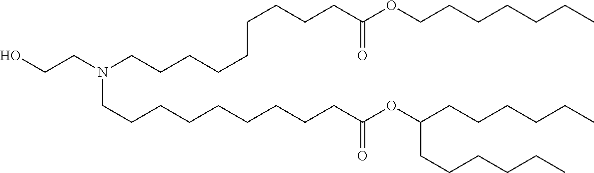 [SMILES: CCCCCCCOC(=O)CCCCCCCCCN(CCO)CCCCCCCCCC(=O)OC(CCCCCC)CCCCCC] |
| I-187 | 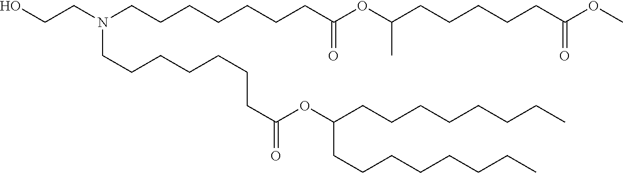 [SMILES: CCCCCCCCC(CCCCCCCC)OC(=O)CCCCCCCN(CCO)CCCCCCCC(=O)OC(C)CCCCCC(=O)OC] |
| I-188 | 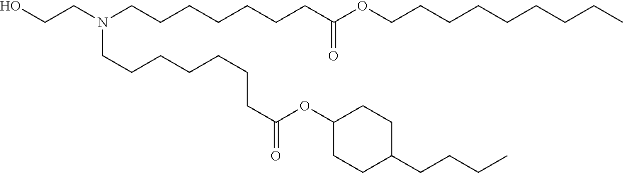 [SMILES: CCCCCCCCCOC(=O)CCCCCCCN(CCO)CCCCCCCC(=O)OC1CCC(CCCC)CC1] |
| I-189 | 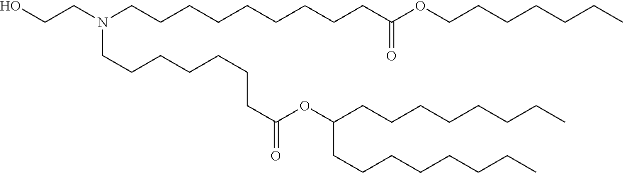 [SMILES: CCCCCCCCC(CCCCCCCC)OC(=O)CCCCCCCN(CCO)CCCCCCCCCC(=O)OCCCCCCC] |
| I-190 | 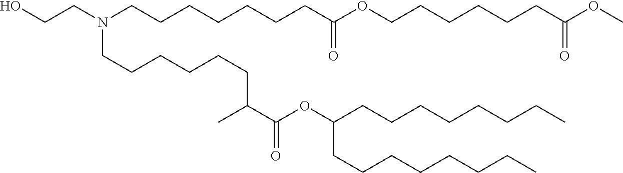 [SMILES: CCCCCCCCC(CCCCCCCC)OC(=O)C(C)CCCCCCN(CCO)CCCCCCCC(=O)OCCCCCCC(=O)OC] |
| I-191 | 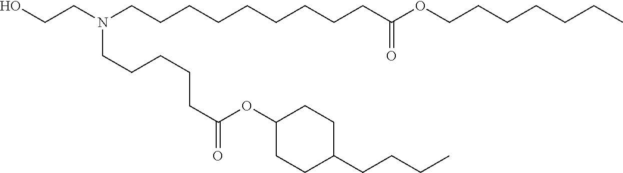 [SMILES: CCCCCCCOC(=O)CCCCCCCCCN(CCO)CCCCCC(=O)OC1CCC(CCCC)CC1] |
| I-192 | 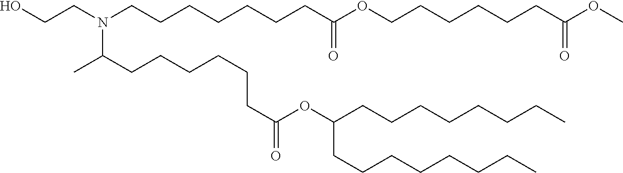 [SMILES: CCCCCCCCC(CCCCCCCC)OC(=O)CCCCCCC(C)N(CCO)CCCCCCCC(=O)OCCCCCCC(=O)OC] |
| I-193 | 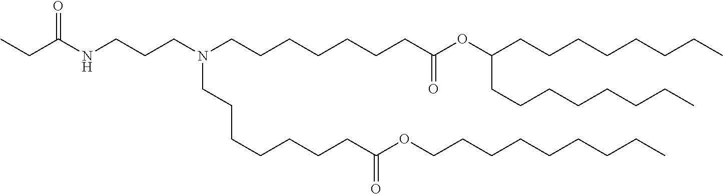 [SMILES: CCCCCCCCCOC(=O)CCCCCCCN(CCCCCCCC(=O)OC(CCCCCCCC)CCCCCCCC)CCCNC(=O)CC] |
| I-194 | 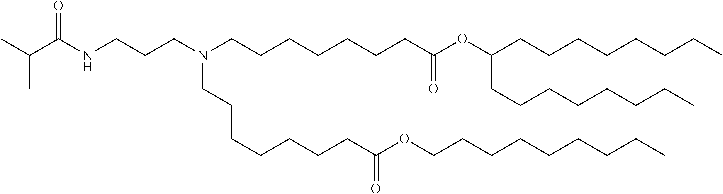 [SMILES: CCCCCCCCCOC(=O)CCCCCCCN(CCCCCCCC(=O)OC(CCCCCCCC)CCCCCCCC)CCCNC(=O)C(C)C] |
| I-195 | 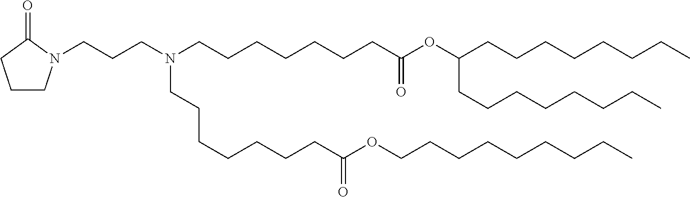 [SMILES: CCCCCCCCCOC(=O)CCCCCCCN(CCCCCCCC(=O)OC(CCCCCCCC)CCCCCCCC)CCCN1CCCC1=O] |
| I-196 | 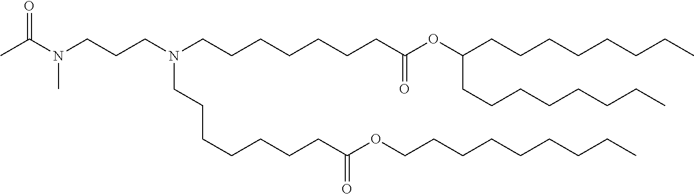 [SMILES: CCCCCCCCCOC(=O)CCCCCCCN(CCCCCCCC(=O)OC(CCCCCCCC)CCCCCCCC)CCCN(C)C(C)=O] |
| I-197 | 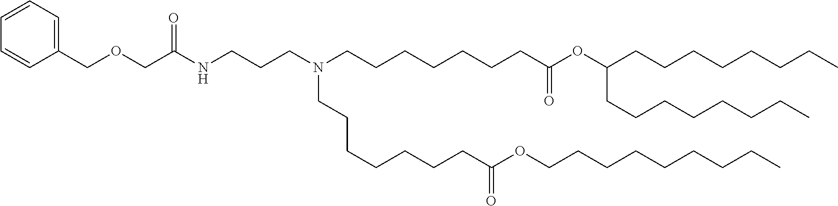 [SMILES: CCCCCCCCCOC(=O)CCCCCCCN(CCCCCCCC(=O)OC(CCCCCCCC)CCCCCCCC)CCCNC(=O)COCc1ccccc1] |
| I-198 | 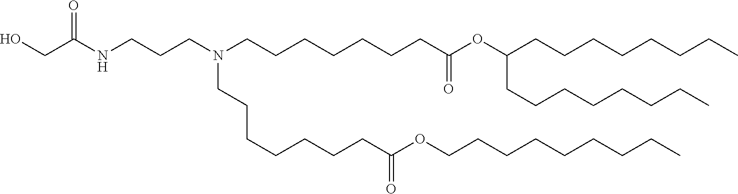 [SMILES: CCCCCCCCCOC(=O)CCCCCCCN(CCCCCCCC(=O)OC(CCCCCCCC)CCCCCCCC)CCCNC(=O)CO] |
| I-199 | 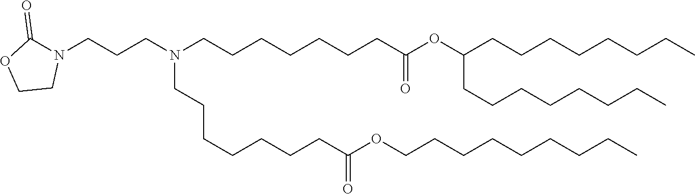 [SMILES: CCCCCCCCCOC(=O)CCCCCCCN(CCCCCCCC(=O)OC(CCCCCCCC)CCCCCCCC)CCCN1CCOC1=O] |
| I-200 | 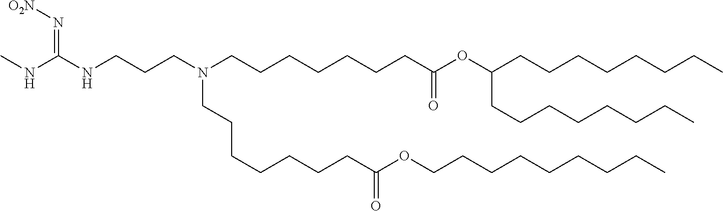 [SMILES: CCCCCCCCCOC(=O)CCCCCCCN(CCCCCCCC(=O)OC(CCCCCCCC)CCCCCCCC)CCCN/C(=N/[N+](=O)[O-])NC] |
| I-201 | 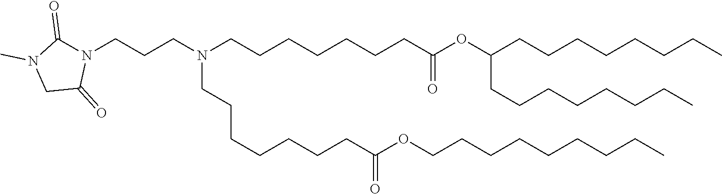 [SMILES: CCCCCCCCCOC(=O)CCCCCCCN(CCCCCCCC(=O)OC(CCCCCCCC)CCCCCCCC)CCCN1C(=O)CN(C)C1=O] |
| I-202 | 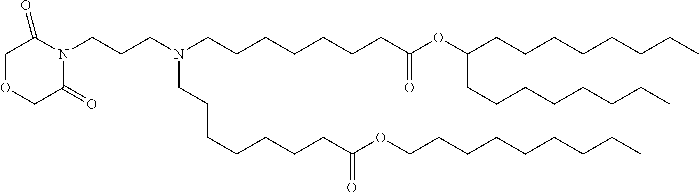 [SMILES: CCCCCCCCCOC(=O)CCCCCCCN(CCCCCCCC(=O)OC(CCCCCCCC)CCCCCCCC)CCCN1C(=O)COCC1=O] |
| I-203 | 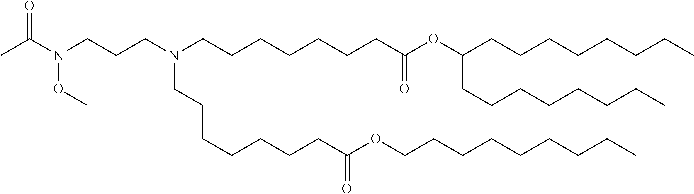 [SMILES: CCCCCCCCCOC(=O)CCCCCCCN(CCCCCCCC(=O)OC(CCCCCCCC)CCCCCCCC)CCCN(OC)C(C)=O] |
| I-204 | 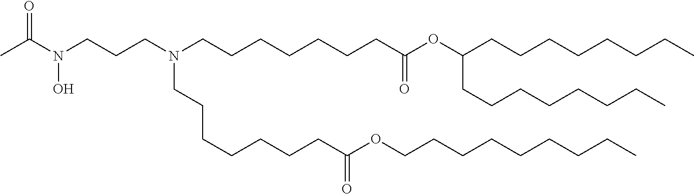 [SMILES: CCCCCCCCCOC(=O)CCCCCCCN(CCCCCCCC(=O)OC(CCCCCCCC)CCCCCCCC)CCCN(O)C(C)=O] |
| I-205 | 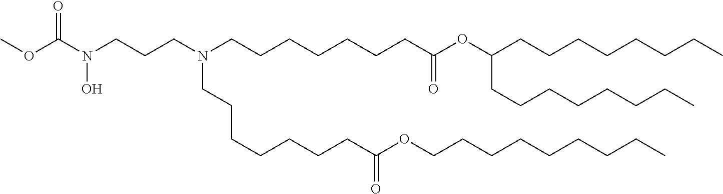 [SMILES: CCCCCCCCCOC(=O)CCCCCCCN(CCCCCCCC(=O)OC(CCCCCCCC)CCCCCCCC)CCCN(O)C(=O)OC] |
| I-206 | 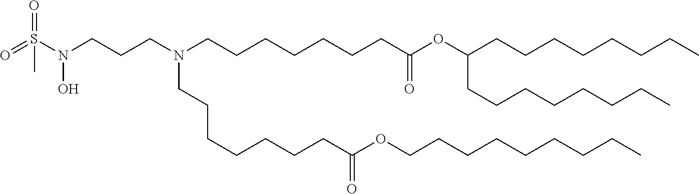 [SMILES: CCCCCCCCCOC(=O)CCCCCCCN(CCCCCCCC(=O)OC(CCCCCCCC)CCCCCCCC)CCCN(O)S(C)(=O)=O] |
| I-207 | 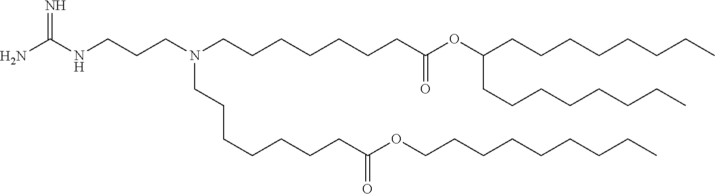 [SMILES: CCCCCCCCCOC(=O)CCCCCCCN(CCCCCCCC(=O)OC(CCCCCCCC)CCCCCCCC)CCCNC(=N)N] |
| I-208 | 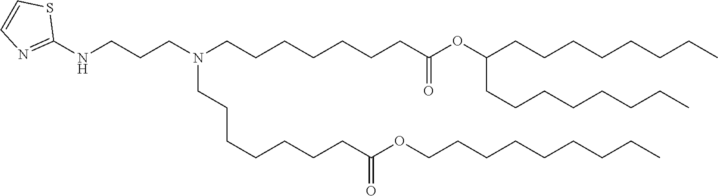 [SMILES: CCCCCCCCCOC(=O)CCCCCCCN(CCCCCCCC(=O)OC(CCCCCCCC)CCCCCCCC)CCCNc1nccs1] |
| I-209 | 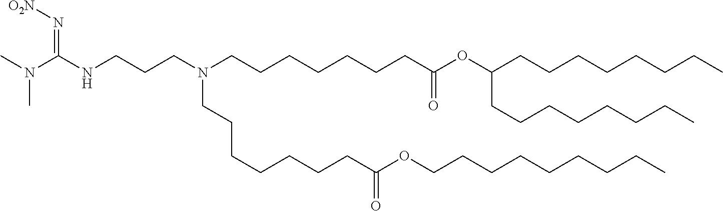 [SMILES: CCCCCCCCCOC(=O)CCCCCCCN(CCCCCCCC(=O)OC(CCCCCCCC)CCCCCCCC)CCCN/C(=N/[N+](=O)[O-])N(C)C] |
| I-210 | 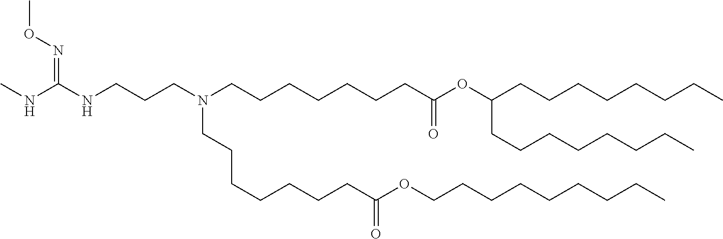 [SMILES: CCCCCCCCCOC(=O)CCCCCCCN(CCCCCCCC(=O)OC(CCCCCCCC)CCCCCCCC)CCCN/C(=N/OC)NC] |
| I-211 | 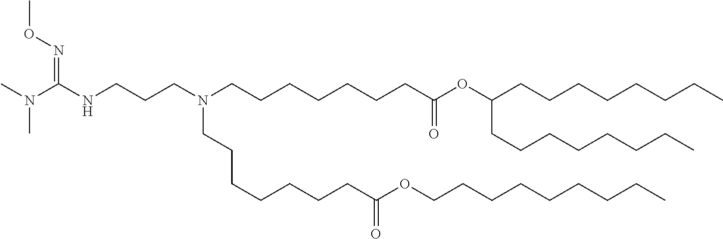 [SMILES: CCCCCCCCCOC(=O)CCCCCCCN(CCCCCCCC(=O)OC(CCCCCCCC)CCCCCCCC)CCCN/C(=N/OC)N(C)C] |
| I-212 | 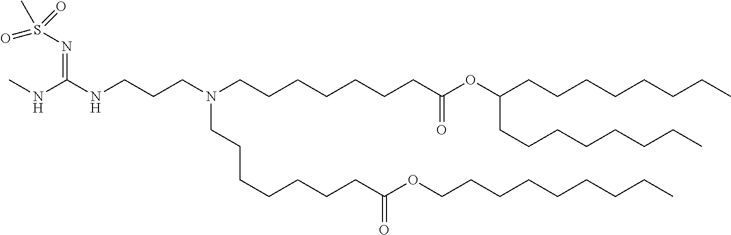 [SMILES: CCCCCCCCCOC(=O)CCCCCCCN(CCCCCCCC(=O)OC(CCCCCCCC)CCCCCCCC)CCCN/C(=N/S(C)(=O)=O)NC] |
| I-213 | 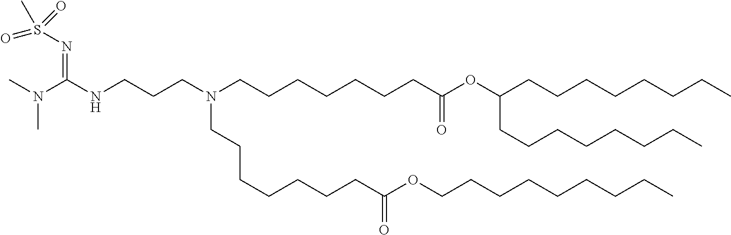 [SMILES: CCCCCCCCCOC(=O)CCCCCCCN(CCCCCCCC(=O)OC(CCCCCCCC)CCCCCCCC)CCCN/C(=N/S(C)(=O)=O)N(C)C] |
| I-214 | 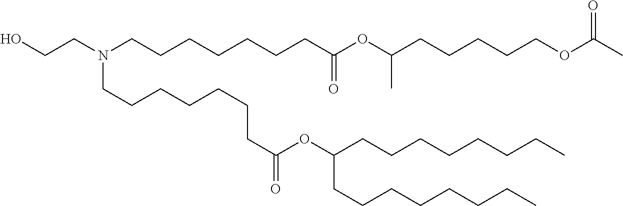 [SMILES: CCCCCCCCC(CCCCCCCC)OC(=O)CCCCCCCN(CCO)CCCCCCCC(=O)OC(C)CCCCCOC(C)=O] |
| I-215 | 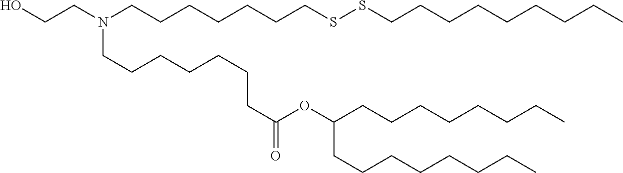 [SMILES: CCCCCCCCCSSCCCCCCCN(CCO)CCCCCCCC(=O)OC(CCCCCCCC)CCCCCCCC] |
| I-216 | 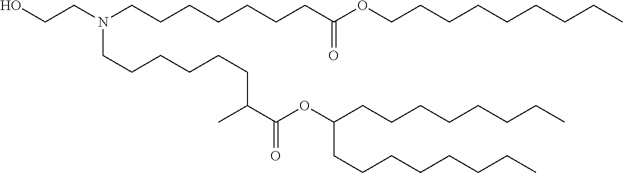 [SMILES: CCCCCCCCCOC(=O)CCCCCCCN(CCO)CCCCCCC(C)C(=O)OC(CCCCCCCC)CCCCCCCC] |
| I-217 | 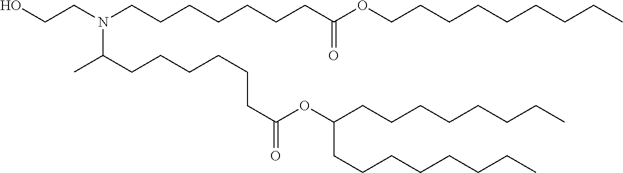 [SMILES: CCCCCCCCCOC(=O)CCCCCCCN(CCO)C(C)CCCCCCC(=O)OC(CCCCCCCC)CCCCCCCC] |
| I-218 | 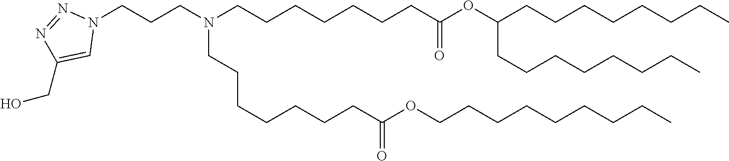 [SMILES: CCCCCCCCCOC(=O)CCCCCCCN(CCCCCCCC(=O)OC(CCCCCCCC)CCCCCCCC)CCCn1cc(CO)nn1] |
| I-219 | 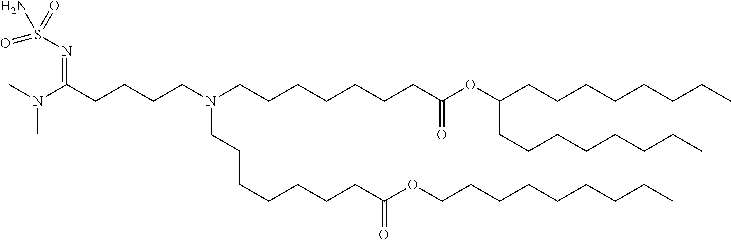 [SMILES: CCCCCCCCCOC(=O)CCCCCCCN(CCCCCCCC(=O)OC(CCCCCCCC)CCCCCCCC)CCCC/C(=N/S(N)(=O)=O)N(C)C] |
| I-220 | 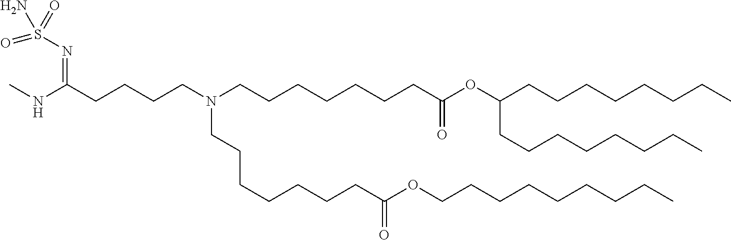 [SMILES: CCCCCCCCCOC(=O)CCCCCCCN(CCCCCCCC(=O)OC(CCCCCCCC)CCCCCCCC)CCCC/C(=N/S(N)(=O)=O)NC] |
| I-221 | 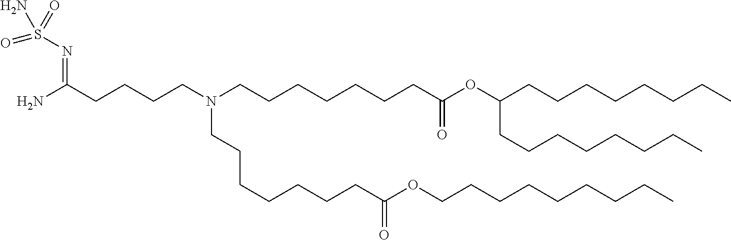 [SMILES: CCCCCCCCCOC(=O)CCCCCCCN(CCCCCCCC(=O)OC(CCCCCCCC)CCCCCCCC)CCCC/C(N)=N/S(N)(=O)=O] |
| I-222 | 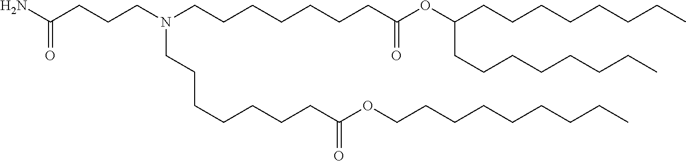 [SMILES: CCCCCCCCCOC(=O)CCCCCCCN(CCCCCCCC(=O)OC(CCCCCCCC)CCCCCCCC)CCCC(N)=O] |
| I-223 | 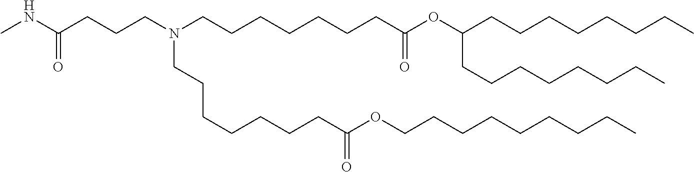 [SMILES: CCCCCCCCCOC(=O)CCCCCCCN(CCCCCCCC(=O)OC(CCCCCCCC)CCCCCCCC)CCCC(=O)NC] |
| I-224 | 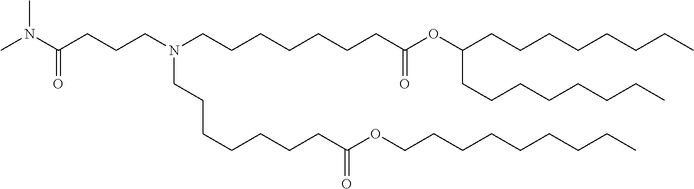 [SMILES: CCCCCCCCCOC(=O)CCCCCCCN(CCCCCCCC(=O)OC(CCCCCCCC)CCCCCCCC)CCCC(=O)N(C)C] |
| I-225 | 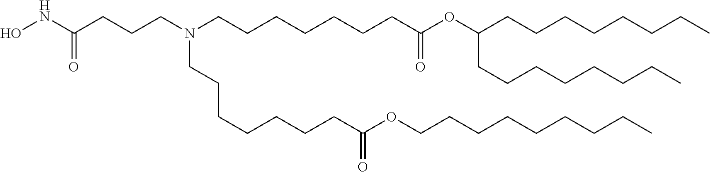 [SMILES: CCCCCCCCCOC(=O)CCCCCCCN(CCCCCCCC(=O)OC(CCCCCCCC)CCCCCCCC)CCCC(=O)NO] |
| I-226 | 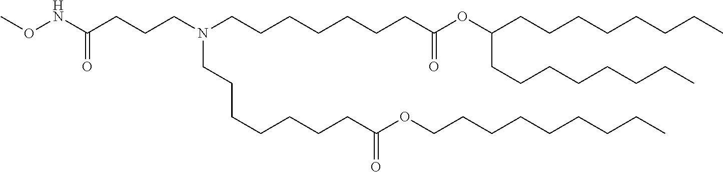 [SMILES: CCCCCCCCCOC(=O)CCCCCCCN(CCCCCCCC(=O)OC(CCCCCCCC)CCCCCCCC)CCCC(=O)NOC] |
| I-227 | 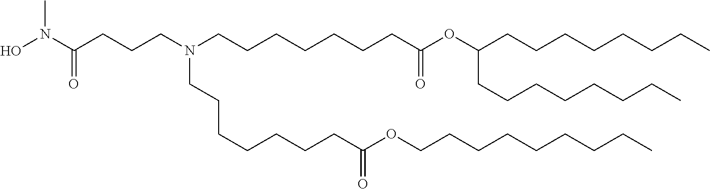 [SMILES: CCCCCCCCCOC(=O)CCCCCCCN(CCCCCCCC(=O)OC(CCCCCCCC)CCCCCCCC)CCCC(=O)N(C)O] |
| I-228 | 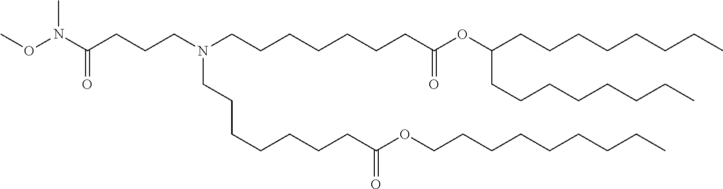 [SMILES: CCCCCCCCCOC(=O)CCCCCCCN(CCCCCCCC(=O)OC(CCCCCCCC)CCCCCCCC)CCCC(=O)N(C)OC] |
| I-229 | 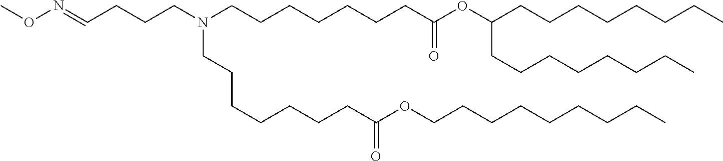 [SMILES: CCCCCCCCCOC(=O)CCCCCCCN(CCC/C=N/OC)CCCCCCCC(=O)OC(CCCCCCCC)CCCCCCCC] |
| I-230 | 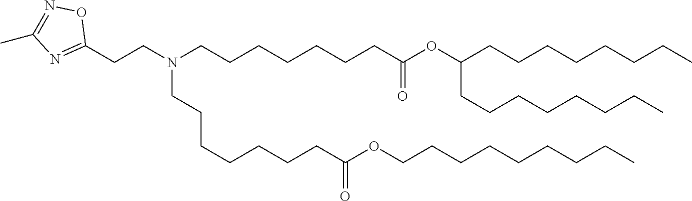 [SMILES: CCCCCCCCCOC(=O)CCCCCCCN(CCCCCCCC(=O)OC(CCCCCCCC)CCCCCCCC)CCc1nc(C)no1] |
| I-231 | 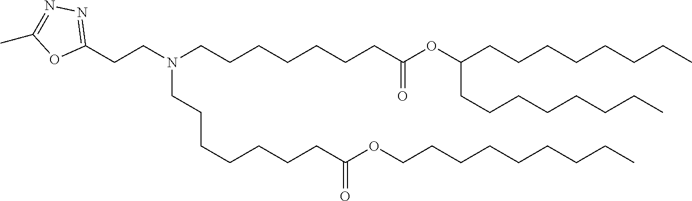 [SMILES: CCCCCCCCCOC(=O)CCCCCCCN(CCCCCCCC(=O)OC(CCCCCCCC)CCCCCCCC)CCc1nnc(C)o1] |
| I-232 | 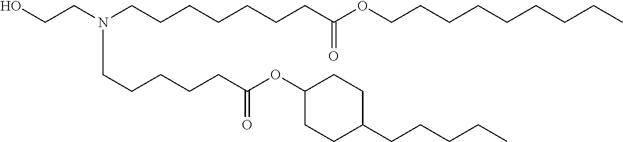 [SMILES: CCCCCCCCCOC(=O)CCCCCCCN(CCO)CCCCCC(=O)OC1CCC(CCCCC)CC1] |
| I-233 | 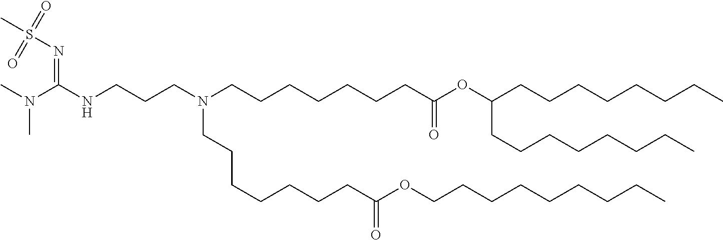 [SMILES: CCCCCCCCCOC(=O)CCCCCCCN(CCCCCCCC(=O)OC(CCCCCCCC)CCCCCCCC)CCCN/C(=N/S(C)(=O)=O)N(C)C] |
| I-234 | 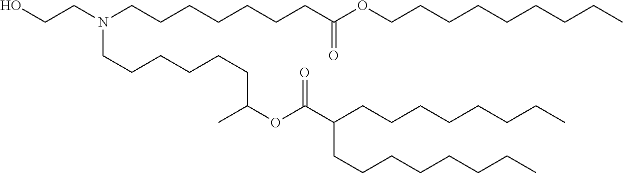 [SMILES: CCCCCCCCCOC(=O)CCCCCCCN(CCO)CCCCCCC(C)OC(=O)C(CCCCCCCC)CCCCCCCC] |
| I-235 | 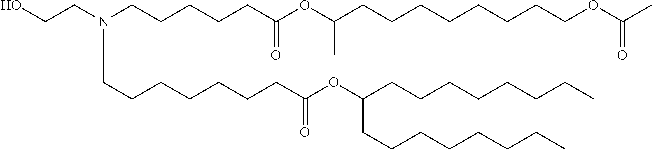 [SMILES: CCCCCCCCC(CCCCCCCC)OC(=O)CCCCCCCN(CCO)CCCCCC(=O)OC(C)CCCCCCCCOC(C)=O] |
| I-236 | 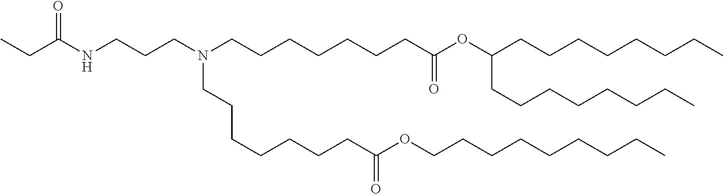 [SMILES: CCCCCCCCCOC(=O)CCCCCCCN(CCCCCCCC(=O)OC(CCCCCCCC)CCCCCCCC)CCCNC(=O)CC] |
| I-237 | 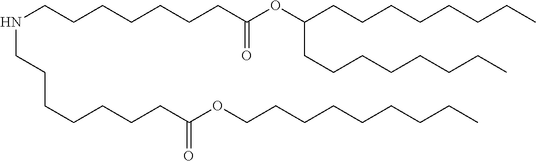 [SMILES: CCCCCCCCCOC(=O)CCCCCCCNCCCCCCCC(=O)OC(CCCCCCCC)CCCCCCCC] |
| I-238 | 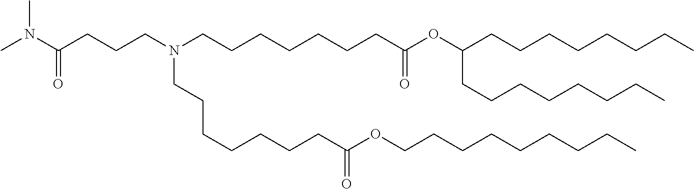 [SMILES: CCCCCCCCCOC(=O)CCCCCCCN(CCCCCCCC(=O)OC(CCCCCCCC)CCCCCCCC)CCCC(=O)N(C)C] |
| I-239 | 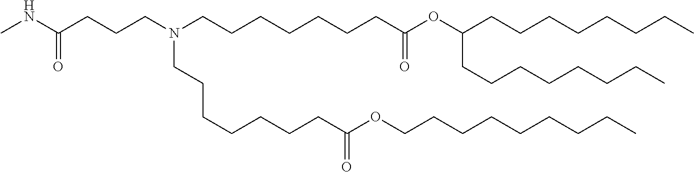 [SMILES: CCCCCCCCCOC(=O)CCCCCCCN(CCCCCCCC(=O)OC(CCCCCCCC)CCCCCCCC)CCCC(=O)NC] |
| I-240 | 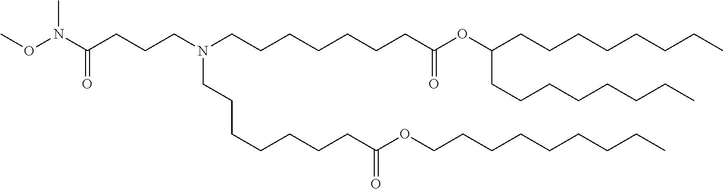 [SMILES: CCCCCCCCCOC(=O)CCCCCCCN(CCCCCCCC(=O)OC(CCCCCCCC)CCCCCCCC)CCCC(=O)N(C)OC] |
| I-241 | 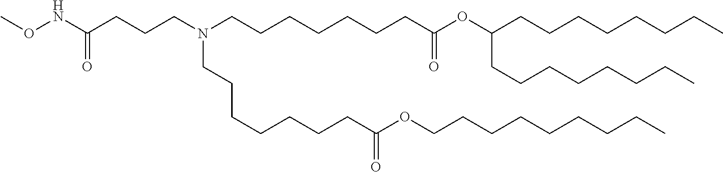 [SMILES: CCCCCCCCCOC(=O)CCCCCCCN(CCCCCCCC(=O)OC(CCCCCCCC)CCCCCCCC)CCCC(=O)NOC] |
| I-242 | 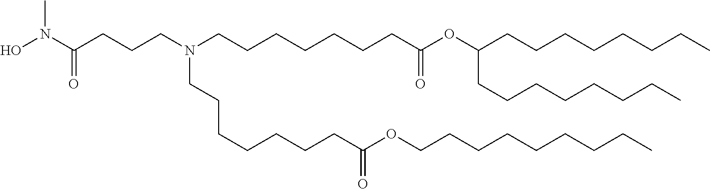 [SMILES: CCCCCCCCCOC(=O)CCCCCCCN(CCCCCCCC(=O)OC(CCCCCCCC)CCCCCCCC)CCCC(=O)N(C)O] |
| I-243 | 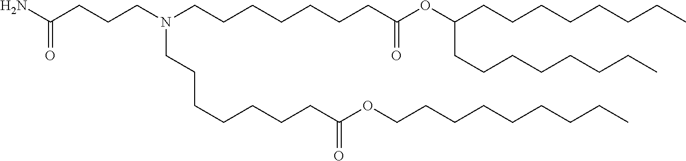 [SMILES: CCCCCCCCCOC(=O)CCCCCCCN(CCCCCCCC(=O)OC(CCCCCCCC)CCCCCCCC)CCCC(N)=O] |
| I-244 | 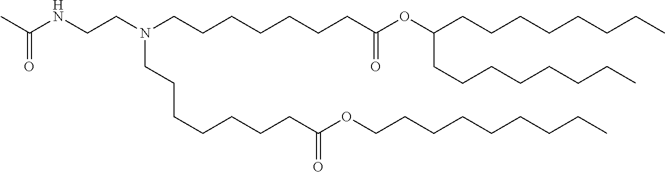 [SMILES: CCCCCCCCCOC(=O)CCCCCCCN(CCCCCCCC(=O)OC(CCCCCCCC)CCCCCCCC)CCNC(C)=O] |
| I-245 | 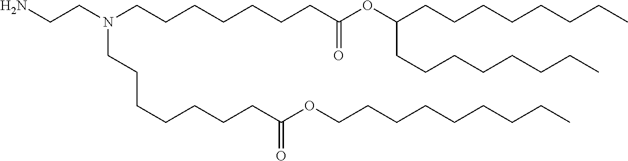 [SMILES: CCCCCCCCCOC(=O)CCCCCCCN(CCN)CCCCCCCC(=O)OC(CCCCCCCC)CCCCCCCC] |
| I-246 | 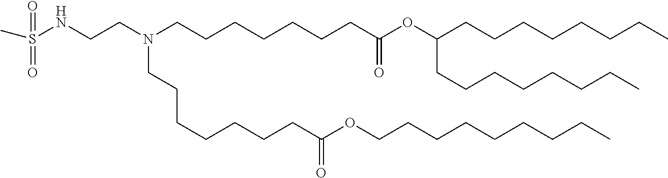 [SMILES: CCCCCCCCCOC(=O)CCCCCCCN(CCCCCCCC(=O)OC(CCCCCCCC)CCCCCCCC)CCNS(C)(=O)=O] |
| I-247 | 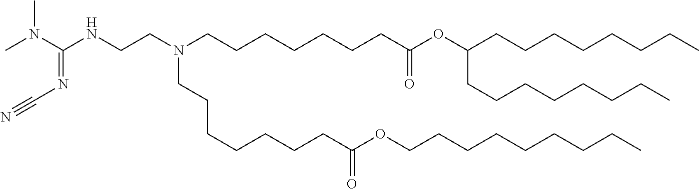 [SMILES: CCCCCCCCCOC(=O)CCCCCCCN(CCCCCCCC(=O)OC(CCCCCCCC)CCCCCCCC)CCN/C(=N/C#N)N(C)C] |
| I-248 | 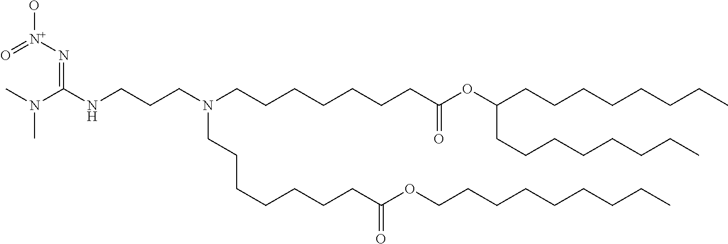 |
| I-249 | 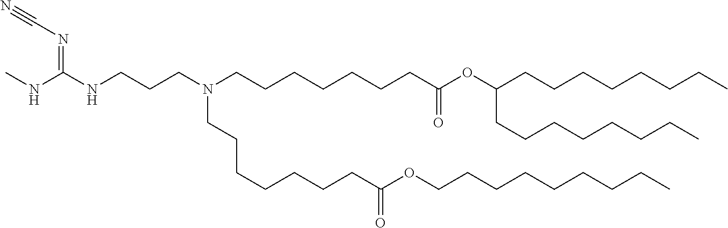 [SMILES: CCCCCCCCCOC(=O)CCCCCCCN(CCCCCCCC(=O)OC(CCCCCCCC)CCCCCCCC)CCCN/C(=N/C#N)NC] |
| I-250 | 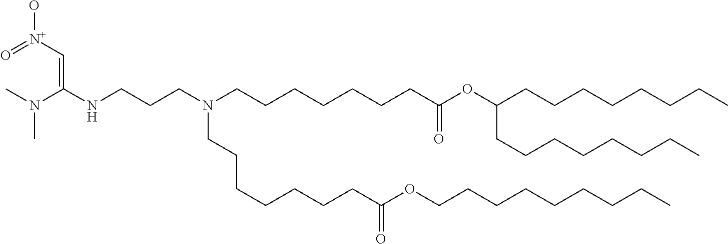 |
| I-251 | 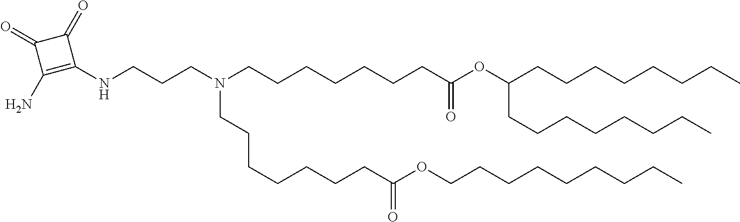 [SMILES: CCCCCCCCCOC(=O)CCCCCCCN(CCCCCCCC(=O)OC(CCCCCCCC)CCCCCCCC)CCCNc1c(N)c(=O)c1=O] |
| I-252 | 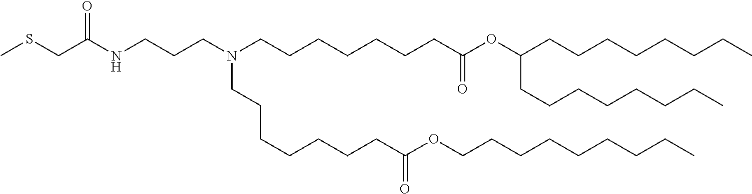 [SMILES: CCCCCCCCCOC(=O)CCCCCCCN(CCCCCCCC(=O)OC(CCCCCCCC)CCCCCCCC)CCCNC(=O)CSC] |
| I-253 | 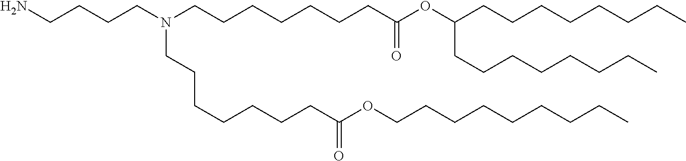 [SMILES: CCCCCCCCCOC(=O)CCCCCCCN(CCCCN)CCCCCCCC(=O)OC(CCCCCCCC)CCCCCCCC] |
| I-254 | 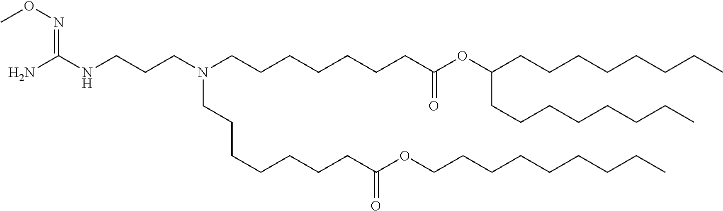 [SMILES: CCCCCCCCCOC(=O)CCCCCCCN(CCCCCCCC(=O)OC(CCCCCCCC)CCCCCCCC)CCCN/C(N)=N/OC] |
| I-255 | 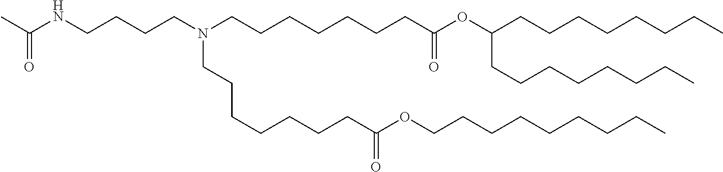 [SMILES: CCCCCCCCCOC(=O)CCCCCCCN(CCCCCCCC(=O)OC(CCCCCCCC)CCCCCCCC)CCCCNC(C)=O] |
| I-256 | 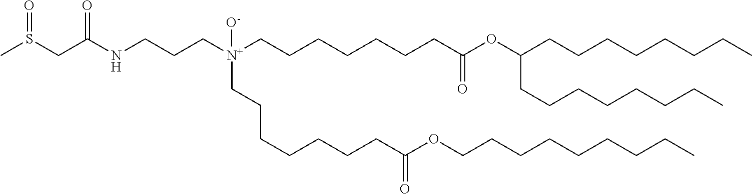 [SMILES: CCCCCCCCCOC(=O)CCCCCCC[N+]([O-])(CCCCCCCC(=O)OC(CCCCCCCC)CCCCCCCC)CCCNC(=O)CS(C)=O] |
| I-257 | 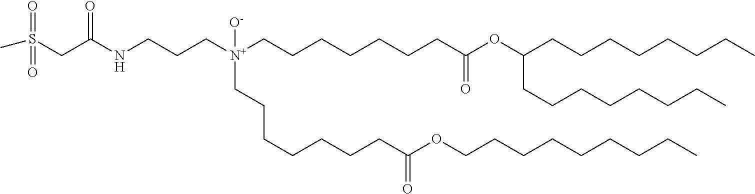 [SMILES: CCCCCCCCCOC(=O)CCCCCCC[N+]([O-])(CCCCCCCC(=O)OC(CCCCCCCC)CCCCCCCC)CCCNC(=O)CS(C)(=O)=O] |
| I-258 | 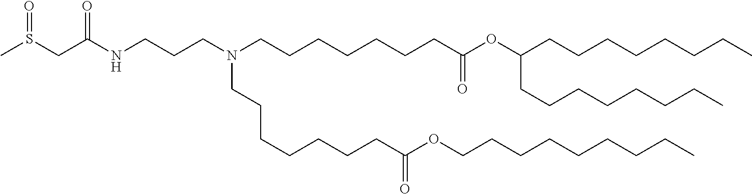 [SMILES: CCCCCCCCCOC(=O)CCCCCCCN(CCCCCCCC(=O)OC(CCCCCCCC)CCCCCCCC)CCCNC(=O)CS(C)=O] |
| I-259 | 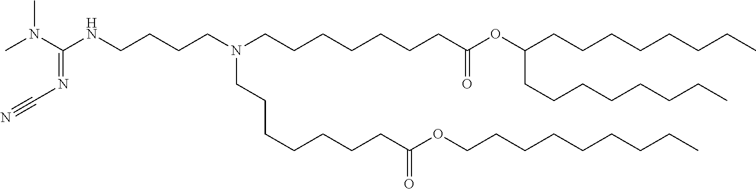 [SMILES: CCCCCCCCCOC(=O)CCCCCCCN(CCCCCCCC(=O)OC(CCCCCCCC)CCCCCCCC)CCCCN/C(=N/C#N)N(C)C] |
| I-260 | 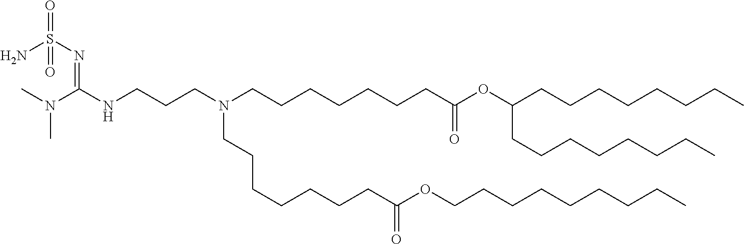 [SMILES: CCCCCCCCCOC(=O)CCCCCCCN(CCCCCCCC(=O)OC(CCCCCCCC)CCCCCCCC)CCCN/C(=N/S(N)(=O)=O)N(C)C] |
| I-261 | 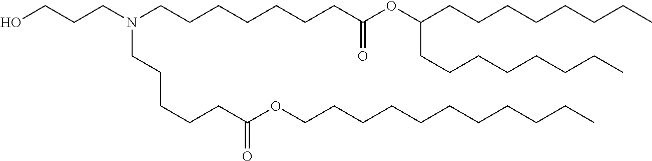 [SMILES: CCCCCCCCCCCOC(=O)CCCCCN(CCCO)CCCCCCCC(=O)OC(CCCCCCCC)CCCCCCCC] |
| I-262 | 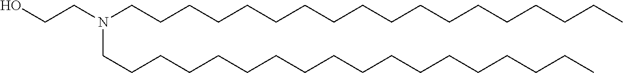 [SMILES: CCCCCCCCCCCCCCCCCCN(CCO)CCCCCCCCCCCCCCCCCC] |
| I-263 | 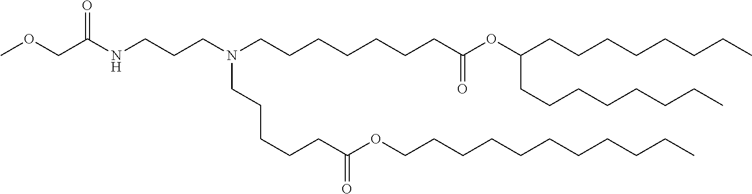 [SMILES: CCCCCCCCCCCOC(=O)CCCCCN(CCCCCCCC(=O)OC(CCCCCCCC)CCCCCCCC)CCCNC(=O)COC] |
| I-264 | 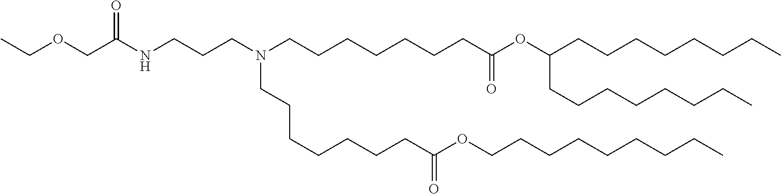 [SMILES: CCCCCCCCCOC(=O)CCCCCCCN(CCCCCCCC(=O)OC(CCCCCCCC)CCCCCCCC)CCCNC(=O)COCC] |
| I-265 | 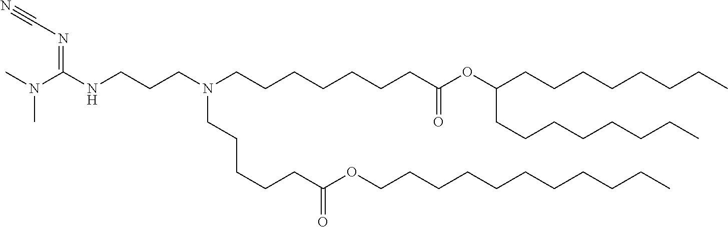 [SMILES: CCCCCCCCCCCOC(=O)CCCCCN(CCCCCCCC(=O)OC(CCCCCCCC)CCCCCCCC)CCCN/C(=N/C#N)N(C)C] |
| I-266 | 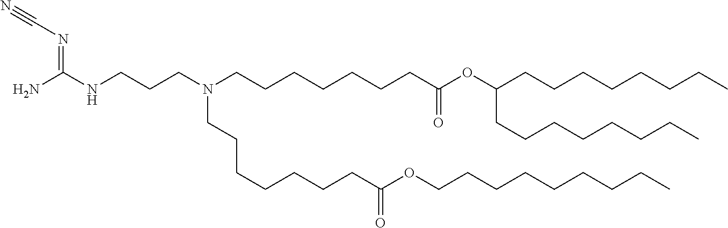 [SMILES: CCCCCCCCCOC(=O)CCCCCCCN(CCCCCCCC(=O)OC(CCCCCCCC)CCCCCCCC)CCCN/C(N)=N/C#N] |
| I-267 | 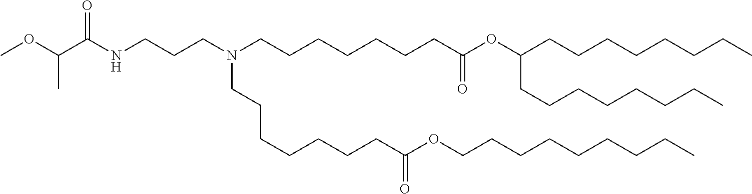 [SMILES: CCCCCCCCCOC(=O)CCCCCCCN(CCCCCCCC(=O)OC(CCCCCCCC)CCCCCCCC)CCCNC(=O)C(C)OC] |
| I-268 | 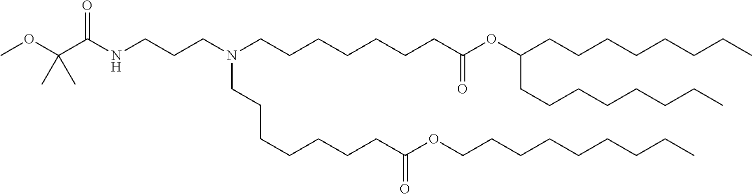 [SMILES: CCCCCCCCCOC(=O)CCCCCCCN(CCCCCCCC(=O)OC(CCCCCCCC)CCCCCCCC)CCCNC(=O)C(C)(C)OC] |
| I-269 | 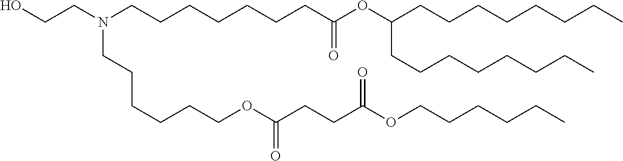 [SMILES: CCCCCCCCC(CCCCCCCC)OC(=O)CCCCCCCN(CCO)CCCCCCOC(=O)CCC(=O)OCCCCCC] |
| I-270 | 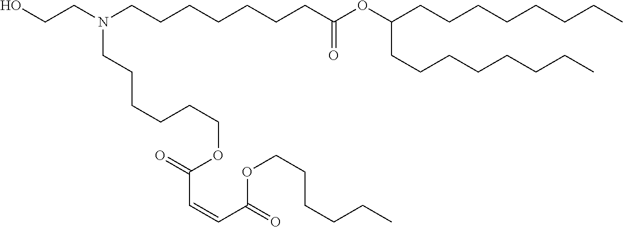 [SMILES: CCCCCCCCC(CCCCCCCC)OC(=O)CCCCCCCN(CCO)CCCCCCOC(=O)/C=C\C(=O)OCCCCCC] |
| I-271 | 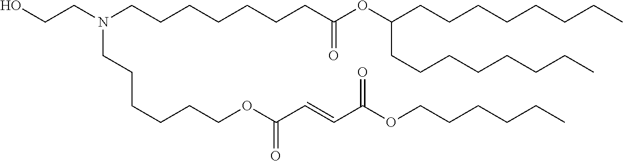 [SMILES: CCCCCCCCC(CCCCCCCC)OC(=O)CCCCCCCN(CCO)CCCCCCOC(=O)/C=C/C(=O)OCCCCCC] |
| I-272 | 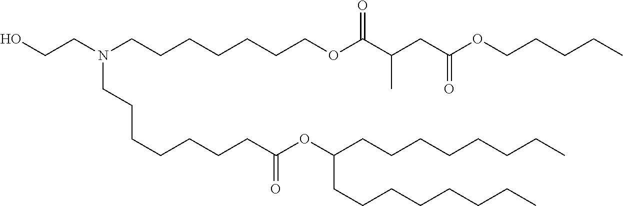 [SMILES: CCCCCCCCC(CCCCCCCC)OC(=O)CCCCCCCN(CCO)CCCCCCCOC(=O)C(C)CC(=O)OCCCCC] |
| I-273 | 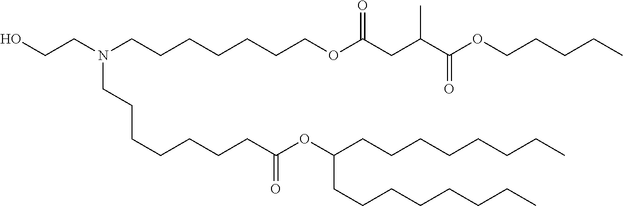 [SMILES: CCCCCCCCC(CCCCCCCC)OC(=O)CCCCCCCN(CCO)CCCCCCCOC(=O)CC(C)C(=O)OCCCCC] |
| I-274 | 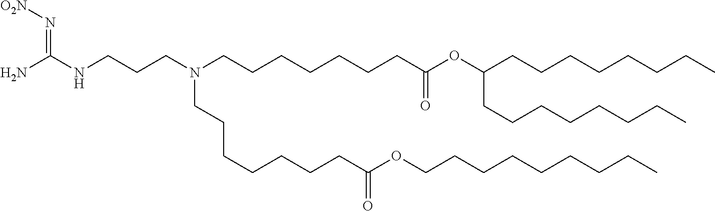 [SMILES: CCCCCCCCCOC(=O)CCCCCCCN(CCCCCCCC(=O)OC(CCCCCCCC)CCCCCCCC)CCCN/C(N)=N/[N+](=O)[O-]] |
| I-275 | 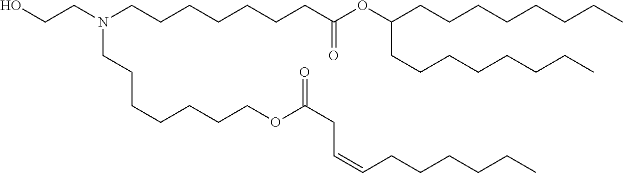 [SMILES: CCCCCC/C=C\CC(=O)OCCCCCCCN(CCO)CCCCCCCC(=O)OC(CCCCCCCC)CCCCCCCC] |
| I-276 | 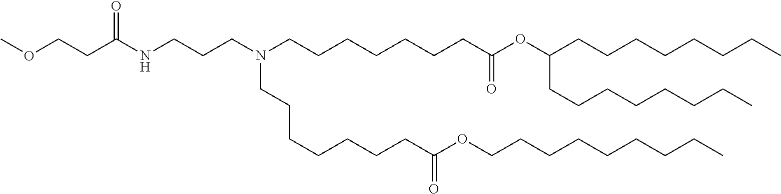 [SMILES: CCCCCCCCCOC(=O)CCCCCCCN(CCCCCCCC(=O)OC(CCCCCCCC)CCCCCCCC)CCCNC(=O)CCOC] |
| I-277 | 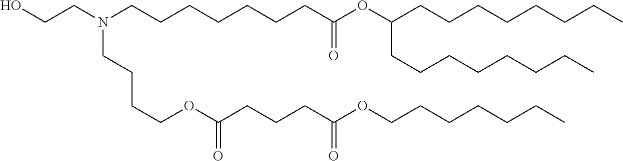 [SMILES: CCCCCCCCC(CCCCCCCC)OC(=O)CCCCCCCN(CCO)CCCCOC(=O)CCCC(=O)OCCCCCCC] |
| I-278 | 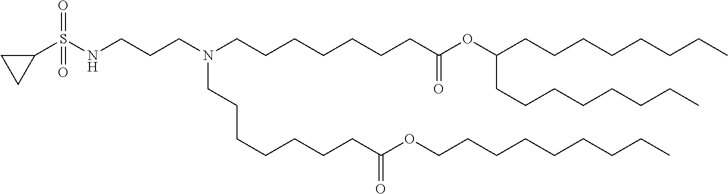 [SMILES: CCCCCCCCCOC(=O)CCCCCCCN(CCCCCCCC(=O)OC(CCCCCCCC)CCCCCCCC)CCCNS(=O)(=O)C1CC1] |
| I-279 | 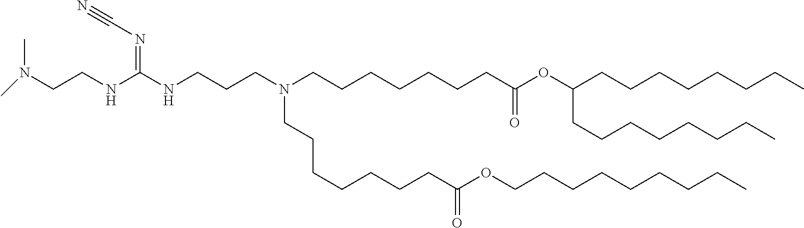 [SMILES: CCCCCCCCCOC(=O)CCCCCCCN(CCCCCCCC(=O)OC(CCCCCCCC)CCCCCCCC)CCCN/C(=N/C#N)NCCN(C)C] |
| I-280 | 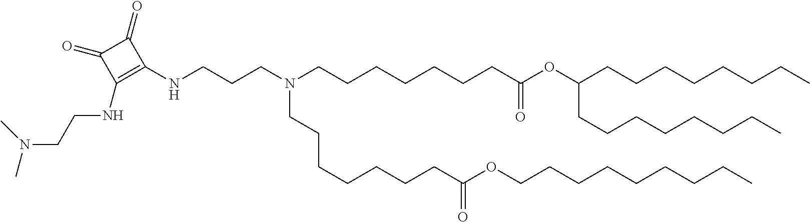 [SMILES: CCCCCCCCCOC(=O)CCCCCCCN(CCCCCCCC(=O)OC(CCCCCCCC)CCCCCCCC)CCCNc1c(NCCN(C)C)c(=O)c1=O] |
| I-281 | 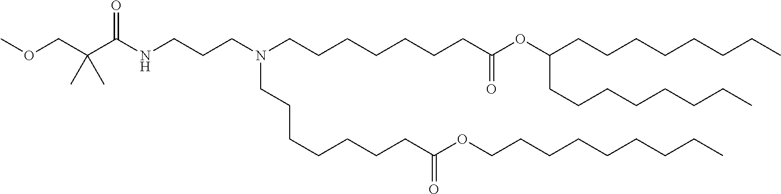 [SMILES: CCCCCCCCCOC(=O)CCCCCCCN(CCCCCCCC(=O)OC(CCCCCCCC)CCCCCCCC)CCCNC(=O)C(C)(C)COC] |
| I-282 | 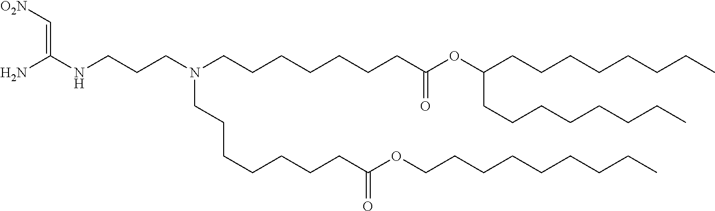 [SMILES: CCCCCCCCCOC(=O)CCCCCCCN(CCCCCCCC(=O)OC(CCCCCCCC)CCCCCCCC)CCCN/C(N)=C/[N+](=O)[O-]] |
| I-283 | 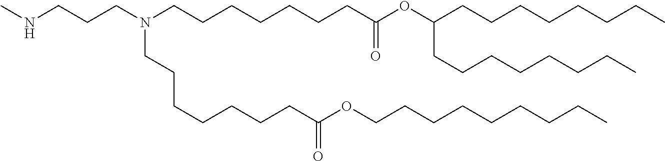 [SMILES: CCCCCCCCCOC(=O)CCCCCCCN(CCCCCCCC(=O)OC(CCCCCCCC)CCCCCCCC)CCCNC] |
| I-284 | 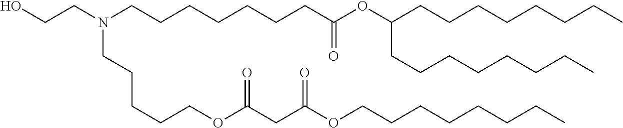 [SMILES: CCCCCCCCOC(=O)CC(=O)OCCCCCN(CCO)CCCCCCCC(=O)OC(CCCCCCCC)CCCCCCCC] |
| I-285 | 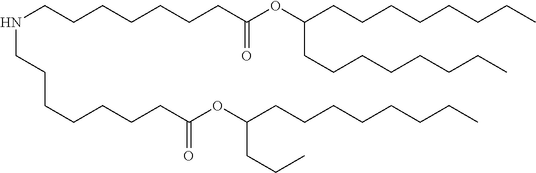 [SMILES: CCCCCCCCC(CCC)OC(=O)CCCCCCCNCCCCCCCC(=O)OC(CCCCCCCC)CCCCCCCC] |
| I-286 | 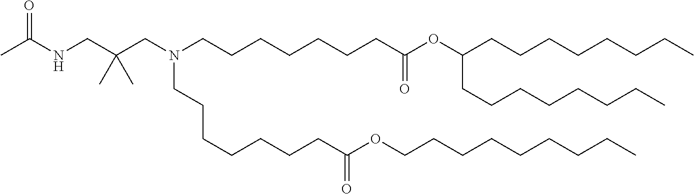 [SMILES: CCCCCCCCCOC(=O)CCCCCCCN(CCCCCCCC(=O)OC(CCCCCCCC)CCCCCCCC)CC(C)(C)CNC(C)=O] |
| I-287 | 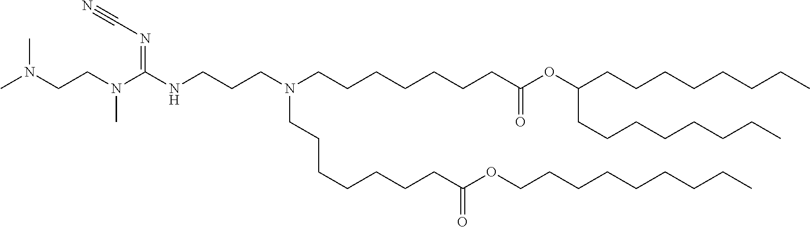 [SMILES: CCCCCCCCCOC(=O)CCCCCCCN(CCCCCCCC(=O)OC(CCCCCCCC)CCCCCCCC)CCCN/C(=N/C#N)N(C)CCN(C)C] |
| I-288 | 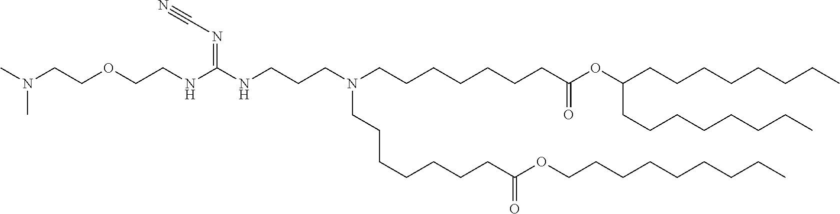 [SMILES: CCCCCCCCCOC(=O)CCCCCCCN(CCCCCCCC(=O)OC(CCCCCCCC)CCCCCCCC)CCCN/C(=N/C#N)NCCOCCN(C)C] |
| I-289 | 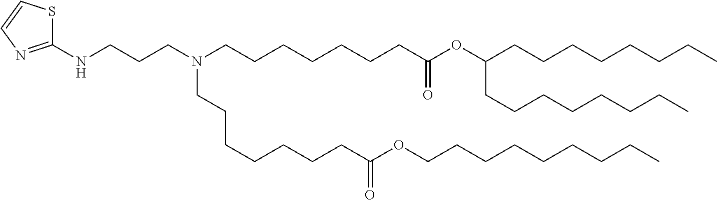 [SMILES: CCCCCCCCCOC(=O)CCCCCCCN(CCCCCCCC(=O)OC(CCCCCCCC)CCCCCCCC)CCCNc1nccs1] |
| I-290 | 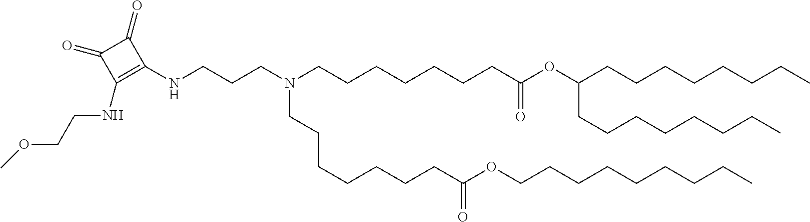 [SMILES: CCCCCCCCCOC(=O)CCCCCCCN(CCCCCCCC(=O)OC(CCCCCCCC)CCCCCCCC)CCCNc1c(NCCOC)c(=O)c1=O] |
| I-291 | 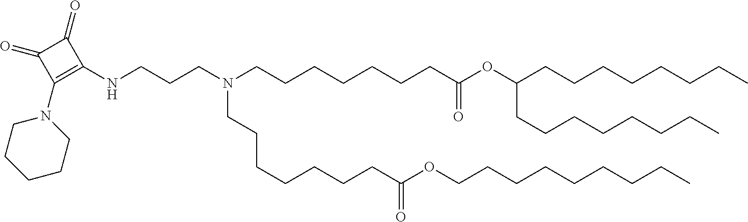 [SMILES: CCCCCCCCCOC(=O)CCCCCCCN(CCCCCCCC(=O)OC(CCCCCCCC)CCCCCCCC)CCCNc1c(N2CCCCC2)c(=O)c1=O] |
| I-292 | 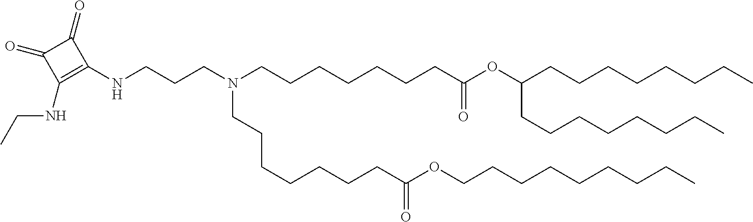 [SMILES: CCCCCCCCCOC(=O)CCCCCCCN(CCCCCCCC(=O)OC(CCCCCCCC)CCCCCCCC)CCCNc1c(NCC)c(=O)c1=O] |
| I-293 | 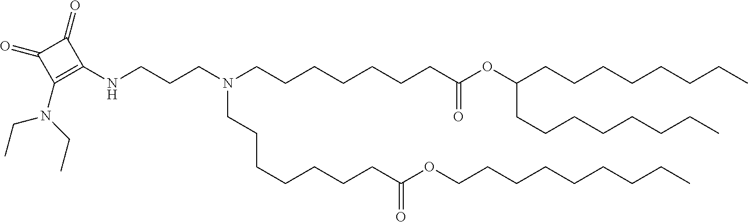 [SMILES: CCCCCCCCCOC(=O)CCCCCCCN(CCCCCCCC(=O)OC(CCCCCCCC)CCCCCCCC)CCCNc1c(N(CC)CC)c(=O)c1=O] |
| I-294 | 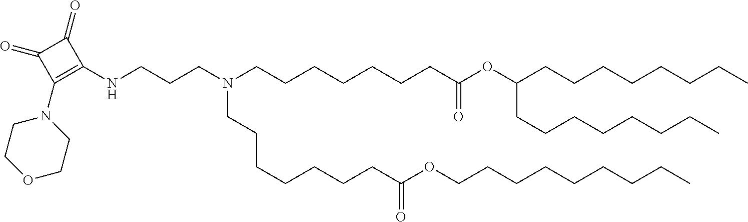 [SMILES: CCCCCCCCCOC(=O)CCCCCCCN(CCCCCCCC(=O)OC(CCCCCCCC)CCCCCCCC)CCCNc1c(N2CCOCC2)c(=O)c1=O] |
| I-295 | 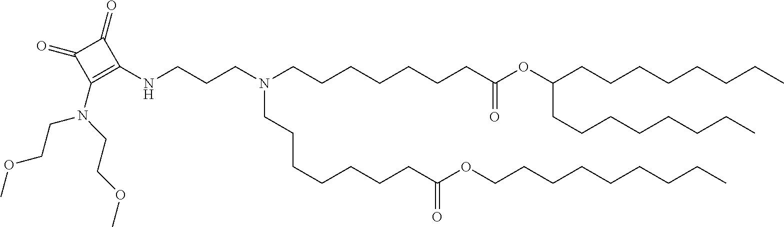 [SMILES: CCCCCCCCCOC(=O)CCCCCCCN(CCCCCCCC(=O)OC(CCCCCCCC)CCCCCCCC)CCCNc1c(N(CCOC)CCOC)c(=O)c1=O] |
| I-296 | 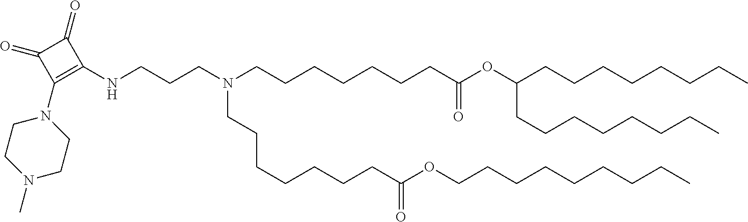 [SMILES: CCCCCCCCCOC(=O)CCCCCCCN(CCCCCCCC(=O)OC(CCCCCCCC)CCCCCCCC)CCCNc1c(N2CCN(C)CC2)c(=O)c1=O] |
| I-297 | 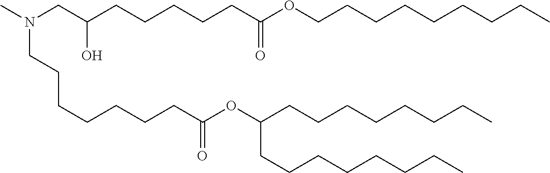 [SMILES: CCCCCCCCCOC(=O)CCCCCC(O)CN(C)CCCCCCCC(=O)OC(CCCCCCCC)CCCCCCCC] |
| I-298 | 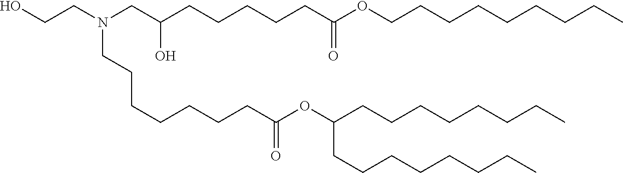 [SMILES: CCCCCCCCCOC(=O)CCCCCC(O)CN(CCO)CCCCCCCC(=O)OC(CCCCCCCC)CCCCCCCC] |
| I-299 | 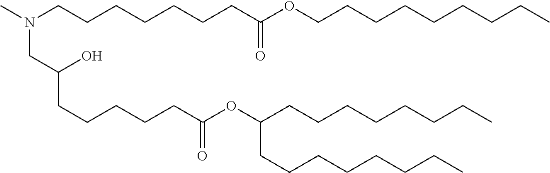 [SMILES: CCCCCCCCCOC(=O)CCCCCCCN(C)CC(O)CCCCCC(=O)OC(CCCCCCCC)CCCCCCCC] |
| I-300 | 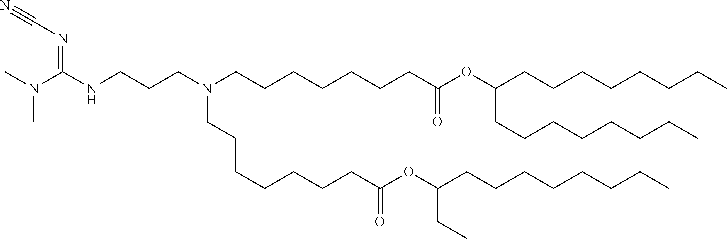 [SMILES: CCCCCCCCC(CC)OC(=O)CCCCCCCN(CCCCCCCC(=O)OC(CCCCCCCC)CCCCCCCC)CCCN/C(=N/C#N)N(C)C] |
| I-301 | 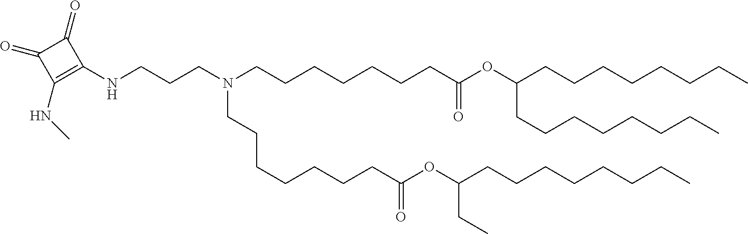 [SMILES: CCCCCCCCC(CC)OC(=O)CCCCCCCN(CCCCCCCC(=O)OC(CCCCCCCC)CCCCCCCC)CCCNc1c(NC)c(=O)c1=O] |
| I-302 | 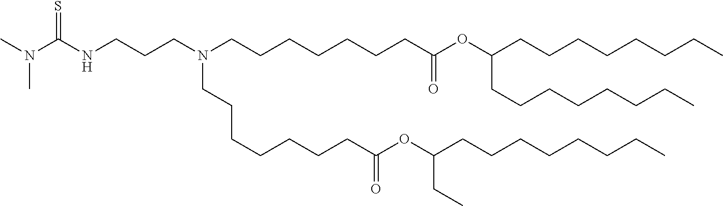 [SMILES: CCCCCCCCC(CC)OC(=O)CCCCCCCN(CCCCCCCC(=O)OC(CCCCCCCC)CCCCCCCC)CCCNC(=S)N(C)C] |
| I-303 | 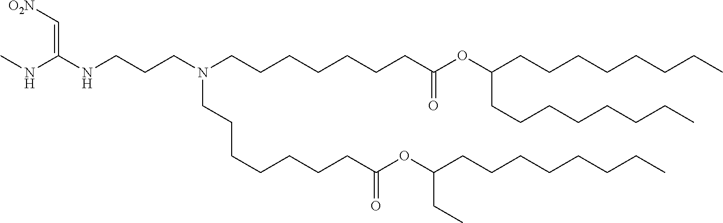 [SMILES: CCCCCCCCC(CC)OC(=O)CCCCCCCN(CCCCCCCC(=O)OC(CCCCCCCC)CCCCCCCC)CCCN/C(=C/[N+](=O)[O-])NC] |
| I-304 | 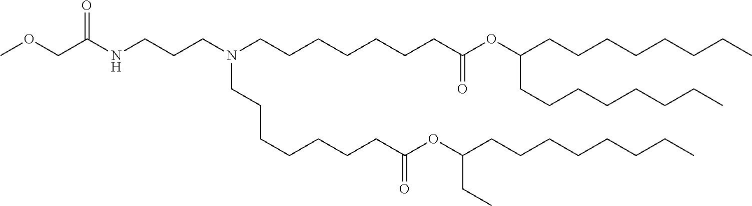 [SMILES: CCCCCCCCC(CC)OC(=O)CCCCCCCN(CCCCCCCC(=O)OC(CCCCCCCC)CCCCCCCC)CCCNC(=O)COC] |
| I-305 | 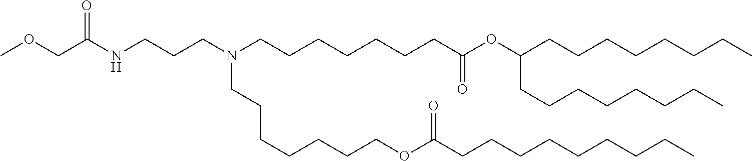 [SMILES: CCCCCCCCCC(=O)OCCCCCCCN(CCCCCCCC(=O)OC(CCCCCCCC)CCCCCCCC)CCCNC(=O)COC] |
| I-306 | 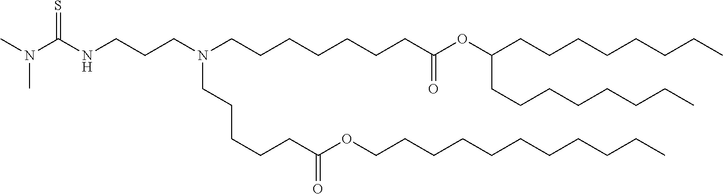 [SMILES: CCCCCCCCCCCOC(=O)CCCCCN(CCCCCCCC(=O)OC(CCCCCCCC)CCCCCCCC)CCCNC(=S)N(C)C] |
| I-307 | 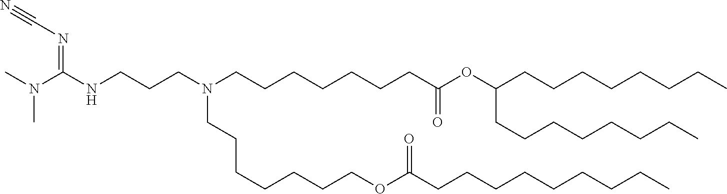 [SMILES: CCCCCCCCCC(=O)OCCCCCCCN(CCCCCCCC(=O)OC(CCCCCCCC)CCCCCCCC)CCCN/C(=N/C#N)N(C)C] |
| I-308 | 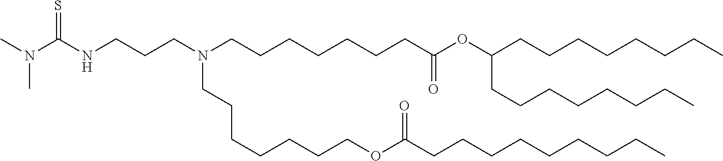 [SMILES: CCCCCCCCCC(=O)OCCCCCCCN(CCCCCCCC(=O)OC(CCCCCCCC)CCCCCCCC)CCCNC(=S)N(C)C] |
| I-309 | 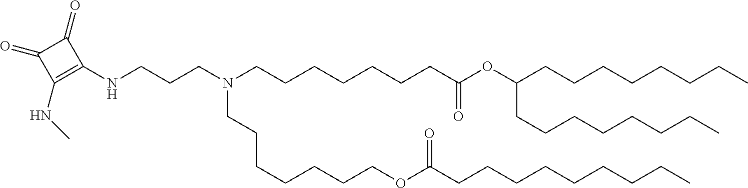 [SMILES: CCCCCCCCCC(=O)OCCCCCCCN(CCCCCCCC(=O)OC(CCCCCCCC)CCCCCCCC)CCCNc1c(NC)c(=O)c1=O] |
| I-310 | 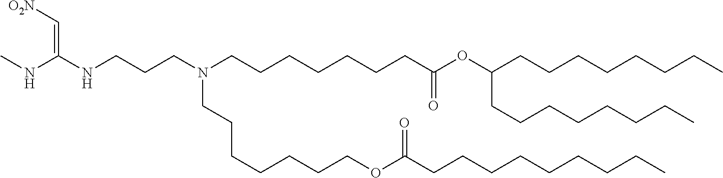 [SMILES: CCCCCCCCCC(=O)OCCCCCCCN(CCCCCCCC(=O)OC(CCCCCCCC)CCCCCCCC)CCCN/C(=C/[N+](=O)[O-])NC] |
| I-311 | 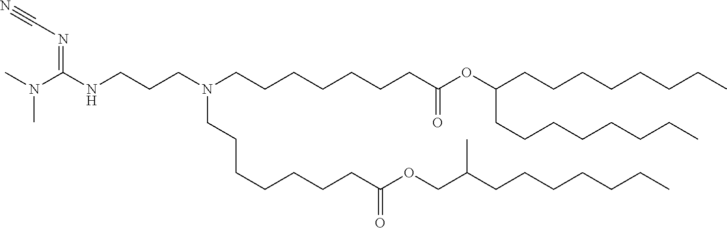 [SMILES: CCCCCCCCC(CCCCCCCC)OC(=O)CCCCCCCN(CCCCCCCC(=O)OCC(C)CCCCCCC)CCCN/C(=N/C#N)N(C)C] |
| I-312 | 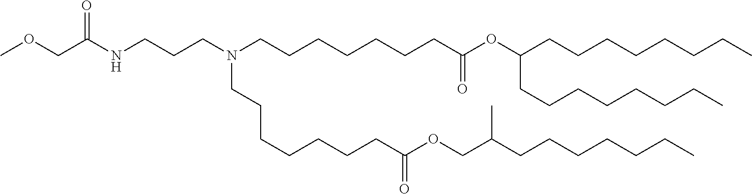 [SMILES: CCCCCCCCC(CCCCCCCC)OC(=O)CCCCCCCN(CCCCCCCC(=O)OCC(C)CCCCCCC)CCCNC(=O)COC] |
| I-313 | 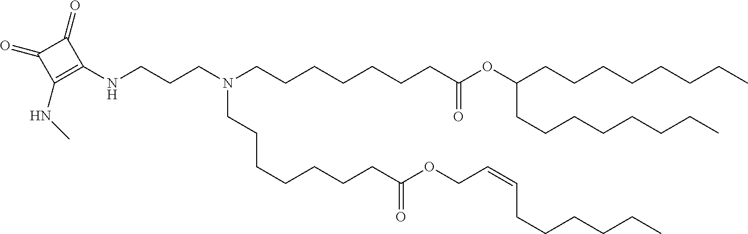 [SMILES: CCCCCC/C=C\COC(=O)CCCCCCCN(CCCCCCCC(=O)OC(CCCCCCCC)CCCCCCCC)CCCNc1c(NC)c(=O)c1=O] |
| I-314 | 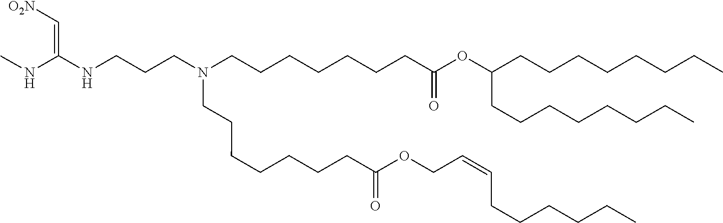 [SMILES: CCCCCC/C=C\COC(=O)CCCCCCCN(CCCCCCCC(=O)OC(CCCCCCCC)CCCCCCCC)CCCN/C(=C/[N+](=O)[O-])NC] |
| I-315 | 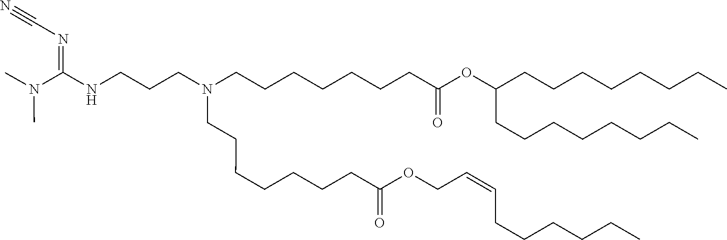 [SMILES: CCCCCC/C=C\COC(=O)CCCCCCCN(CCCCCCCC(=O)OC(CCCCCCCC)CCCCCCCC)CCCN/C(=N/C#N)N(C)C] |
| I-316 | 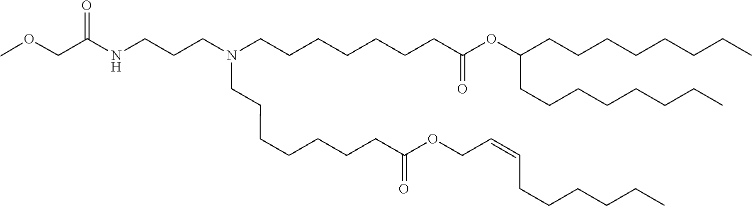 [SMILES: CCCCCC/C=C\COC(=O)CCCCCCCN(CCCCCCCC(=O)OC(CCCCCCCC)CCCCCCCC)CCCNC(=O)COC] |
| I-317 | 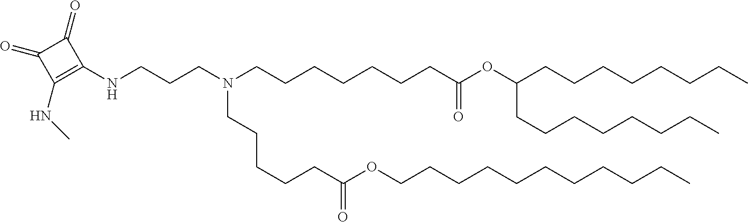 [SMILES: CCCCCCCCCCCOC(=O)CCCCCN(CCCCCCCC(=O)OC(CCCCCCCC)CCCCCCCC)CCCNc1c(NC)c(=O)c1=O] |
| I-318 | 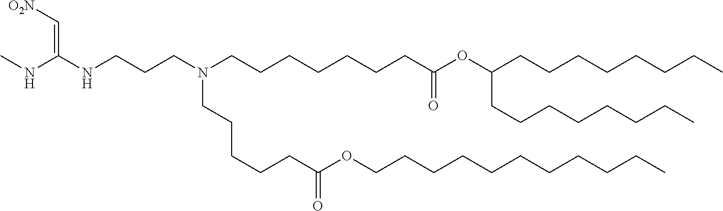 [SMILES: CCCCCCCCCCCOC(=O)CCCCCN(CCCCCCCC(=O)OC(CCCCCCCC)CCCCCCCC)CCCN/C(=C/[N+](=O)[O-])NC] |
| I-319 | 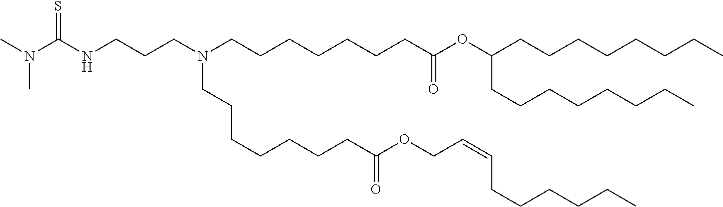 [SMILES: CCCCCC/C=C\COC(=O)CCCCCCCN(CCCCCCCC(=O)OC(CCCCCCCC)CCCCCCCC)CCCNC(=S)N(C)C] |
| I-320 | 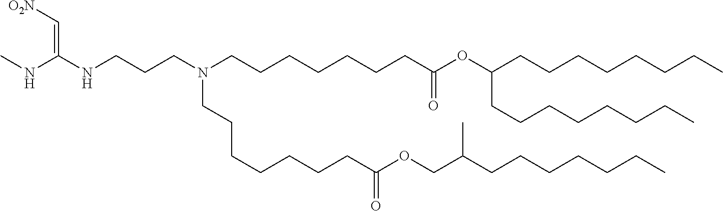 [SMILES: CCCCCCCCC(CCCCCCCC)OC(=O)CCCCCCCN(CCCCCCCC(=O)OCC(C)CCCCCCC)CCCN/C(=C/[N+](=O)[O-])NC] |
| I-321 | 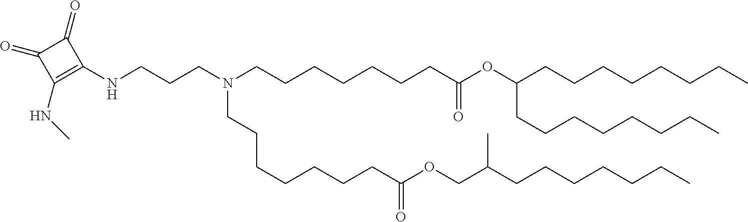 [SMILES: CCCCCCCCC(CCCCCCCC)OC(=O)CCCCCCCN(CCCCCCCC(=O)OCC(C)CCCCCCC)CCCNc1c(NC)c(=O)c1=O] |
| I-322 | 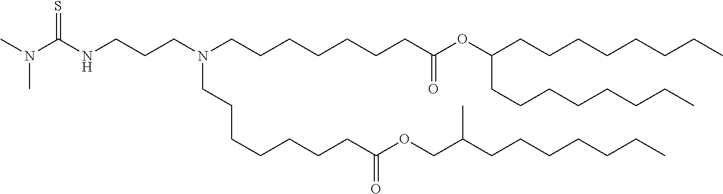 [SMILES: CCCCCCCCC(CCCCCCCC)OC(=O)CCCCCCCN(CCCCCCCC(=O)OCC(C)CCCCCCC)CCCNC(=S)N(C)C] |
| I-323 | 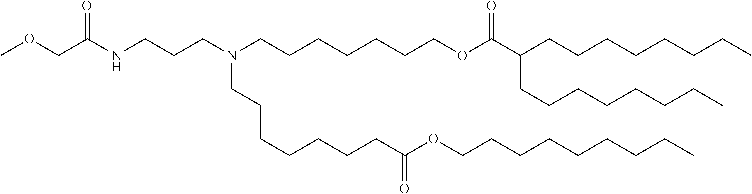 [SMILES: CCCCCCCCCOC(=O)CCCCCCCN(CCCCCCCOC(=O)C(CCCCCCCC)CCCCCCCC)CCCNC(=O)COC] |
| I-324 | 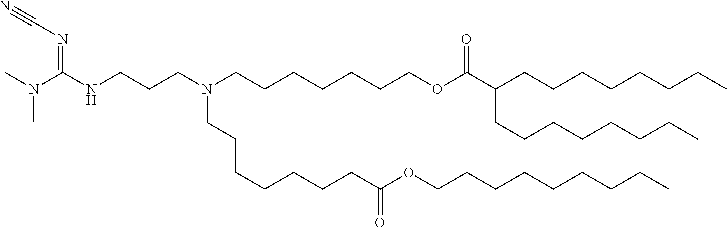 [SMILES: CCCCCCCCCOC(=O)CCCCCCCN(CCCCCCCOC(=O)C(CCCCCCCC)CCCCCCCC)CCCN/C(=N/C#N)N(C)C] |
| I-325 | 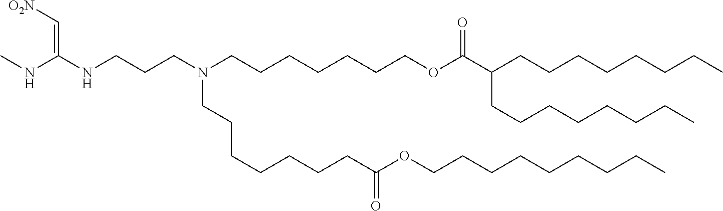 [SMILES: CCCCCCCCCOC(=O)CCCCCCCN(CCCCCCCOC(=O)C(CCCCCCCC)CCCCCCCC)CCCN/C(=C/[N+](=O)[O-])NC] |
| I-326 | 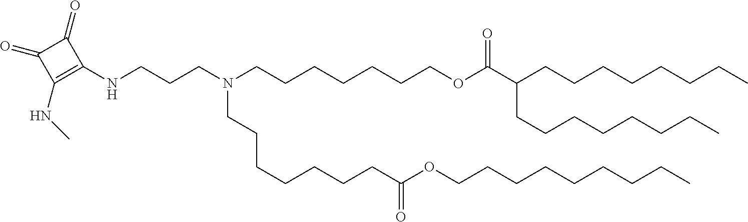 [SMILES: CCCCCCCCCOC(=O)CCCCCCCN(CCCCCCCOC(=O)C(CCCCCCCC)CCCCCCCC)CCCNc1c(NC)c(=O)c1=O] |
| I-327 | 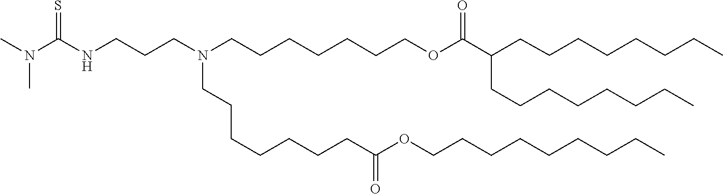 [SMILES: CCCCCCCCCOC(=O)CCCCCCCN(CCCCCCCOC(=O)C(CCCCCCCC)CCCCCCCC)CCCNC(=S)N(C)C] |
| I-328 | 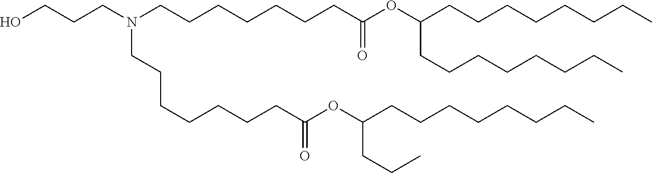 [SMILES: CCCCCCCCC(CCC)OC(=O)CCCCCCCN(CCCO)CCCCCCCC(=O)OC(CCCCCCCC)CCCCCCCC] |
| I-329 | 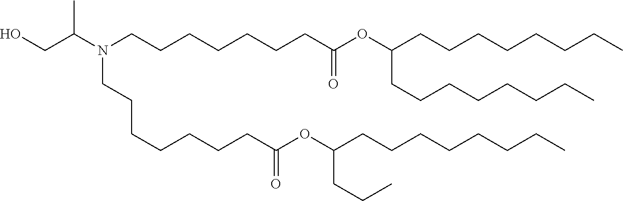 [SMILES: CCCCCCCCC(CCC)OC(=O)CCCCCCCN(CCCCCCCC(=O)OC(CCCCCCCC)CCCCCCCC)C(C)CO] |
| I-330 | 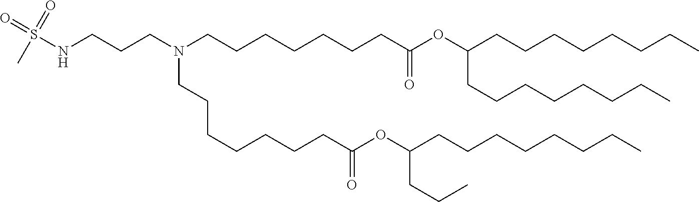 [SMILES: CCCCCCCCC(CCC)OC(=O)CCCCCCCN(CCCCCCCC(=O)OC(CCCCCCCC)CCCCCCCC)CCCNS(C)(=O)=O] |
| I-331 | 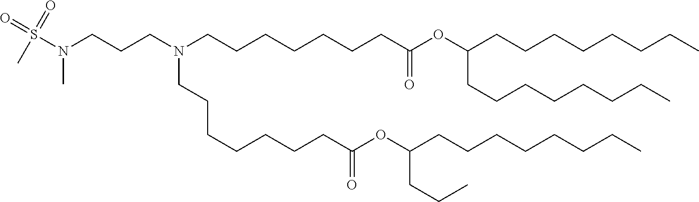 [SMILES: CCCCCCCCC(CCC)OC(=O)CCCCCCCN(CCCCCCCC(=O)OC(CCCCCCCC)CCCCCCCC)CCCN(C)S(C)(=O)=O] |
| I-332 | 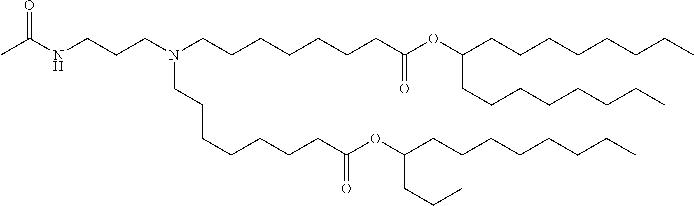 [SMILES: CCCCCCCCC(CCC)OC(=O)CCCCCCCN(CCCCCCCC(=O)OC(CCCCCCCC)CCCCCCCC)CCCNC(C)=O] |
| I-333 | 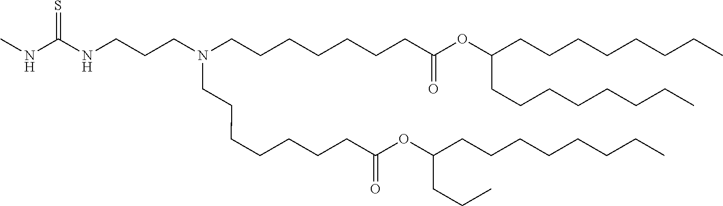 [SMILES: CCCCCCCCC(CCC)OC(=O)CCCCCCCN(CCCCCCCC(=O)OC(CCCCCCCC)CCCCCCCC)CCCNC(=S)NC] |
| I-334 | 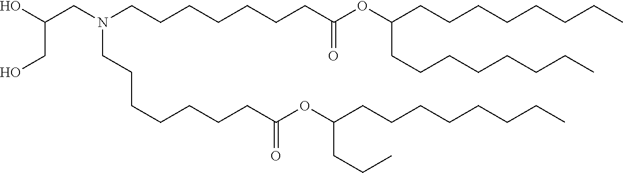 [SMILES: CCCCCCCCC(CCC)OC(=O)CCCCCCCN(CCCCCCCC(=O)OC(CCCCCCCC)CCCCCCCC)CC(O)CO] |
| I-335 | 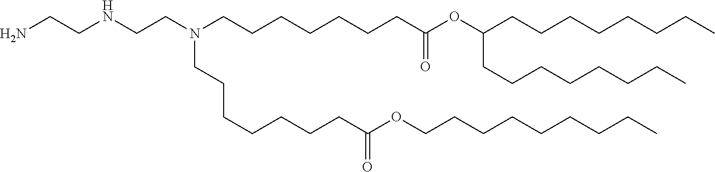 [SMILES: CCCCCCCCCOC(=O)CCCCCCCN(CCCCCCCC(=O)OC(CCCCCCCC)CCCCCCCC)CCNCCN] |
| I-336 | 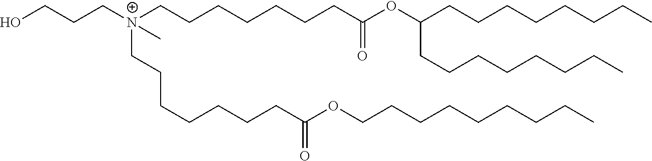 [SMILES: CCCCCCCCCOC(=O)CCCCCCC[N+](C)(CCCO)CCCCCCCC(=O)OC(CCCCCCCC)CCCCCCCC] |
| I-337 | 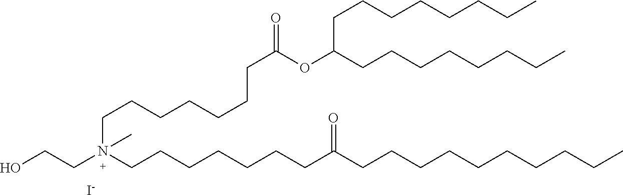 |
| I-338 | 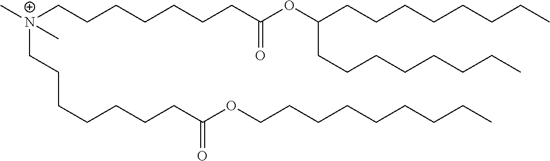 [SMILES: CCCCCCCCCOC(=O)CCCCCCC[N+](C)(C)CCCCCCCC(=O)OC(CCCCCCCC)CCCCCCCC] |
| I-339 | 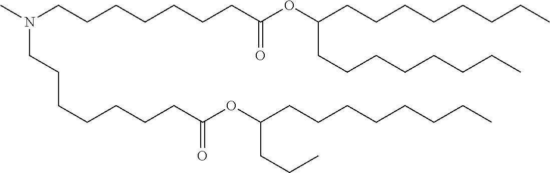 [SMILES: CCCCCCCCC(CCC)OC(=O)CCCCCCCN(C)CCCCCCCC(=O)OC(CCCCCCCC)CCCCCCCC] |
| I-340 | 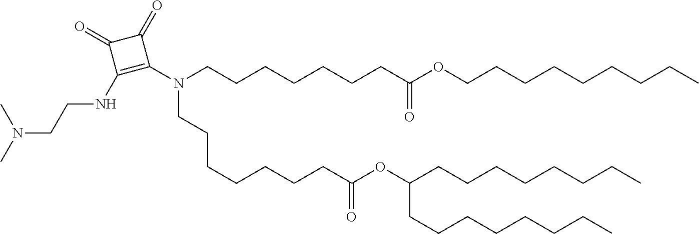 [SMILES: CCCCCCCCCOC(=O)CCCCCCCN(CCCCCCCC(=O)OC(CCCCCCCC)CCCCCCCC)c1c(NCCN(C)C)c(=O)c1=O] |
| I-341 | 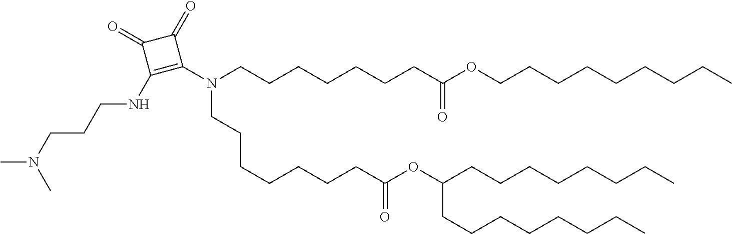 [SMILES: CCCCCCCCCOC(=O)CCCCCCCN(CCCCCCCC(=O)OC(CCCCCCCC)CCCCCCCC)c1c(NCCCN(C)C)c(=O)c1=O] |
| I-342 | 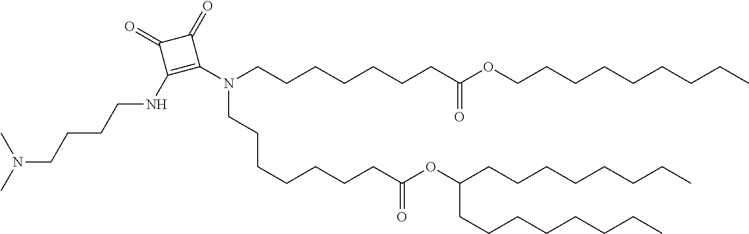 [SMILES: CCCCCCCCCOC(=O)CCCCCCCN(CCCCCCCC(=O)OC(CCCCCCCC)CCCCCCCC)c1c(NCCCCN(C)C)c(=O)c1=O] |
| I-343 | 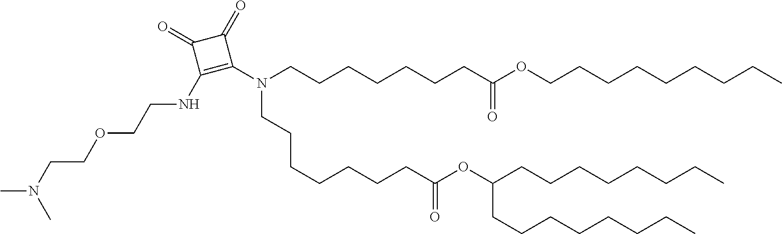 [SMILES: CCCCCCCCCOC(=O)CCCCCCCN(CCCCCCCC(=O)OC(CCCCCCCC)CCCCCCCC)c1c(NCCOCCN(C)C)c(=O)c1=O] |
| I-344 | 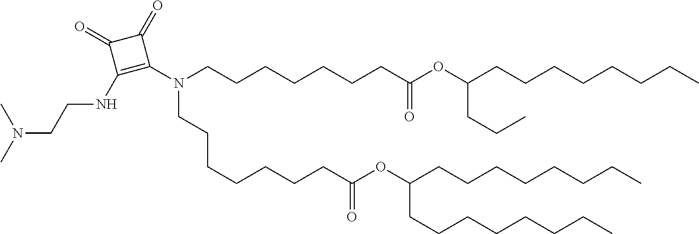 [SMILES: CCCCCCCCC(CCC)OC(=O)CCCCCCCN(CCCCCCCC(=O)OC(CCCCCCCC)CCCCCCCC)c1c(NCCN(C)C)c(=O)c1=O] |
| I-345 | 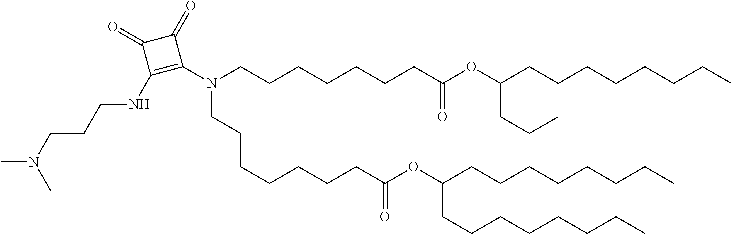 [SMILES: CCCCCCCCC(CCC)OC(=O)CCCCCCCN(CCCCCCCC(=O)OC(CCCCCCCC)CCCCCCCC)c1c(NCCCN(C)C)c(=O)c1=O] |
| I-346 | 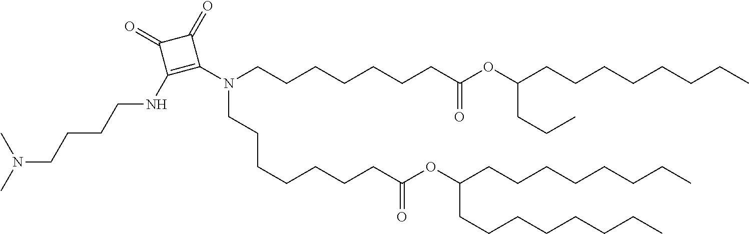 [SMILES: CCCCCCCCC(CCC)OC(=O)CCCCCCCN(CCCCCCCC(=O)OC(CCCCCCCC)CCCCCCCC)c1c(NCCCCN(C)C)c(=O)c1=O] |
| I-347 | 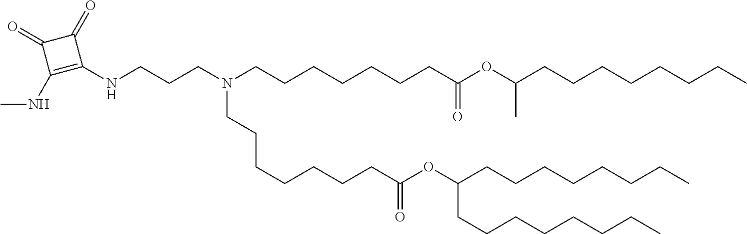 [SMILES: CCCCCCCCC(C)OC(=O)CCCCCCCN(CCCCCCCC(=O)OC(CCCCCCCC)CCCCCCCC)CCCNc1c(NC)c(=O)c1=O] |
| I-348 | 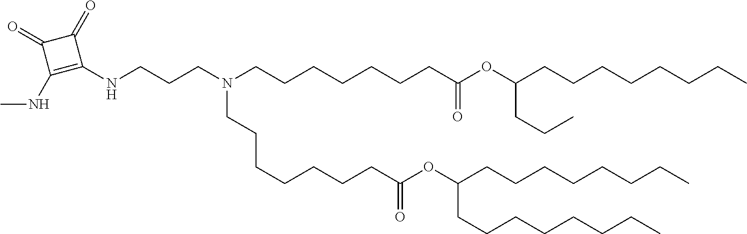 [SMILES: CCCCCCCCC(CCC)OC(=O)CCCCCCCN(CCCCCCCC(=O)OC(CCCCCCCC)CCCCCCCC)CCCNc1c(NC)c(=O)c1=O] |
| I-349 | 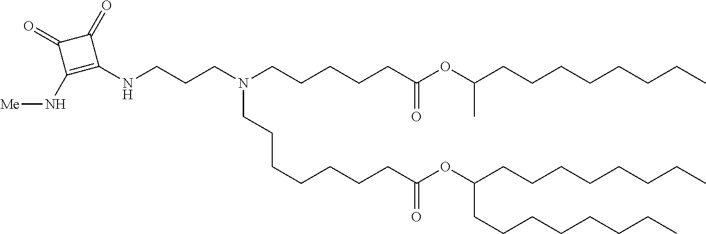 [SMILES: CCCCCCCCC(C)OC(=O)CCCCCN(CCCCCCCC(=O)OC(CCCCCCCC)CCCCCCCC)CCCNc1c(NC)c(=O)c1=O] |
| I-350 | 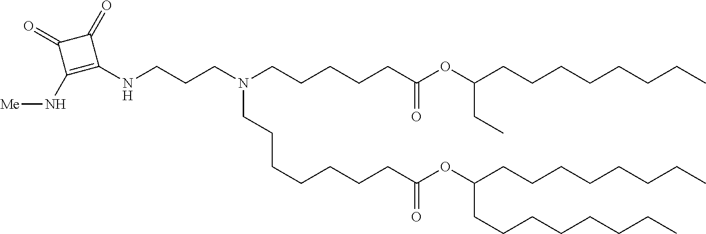 [SMILES: CCCCCCCCC(CC)OC(=O)CCCCCN(CCCCCCCC(=O)OC(CCCCCCCC)CCCCCCCC)CCCNc1c(NC)c(=O)c1=O] |
| I-351 | 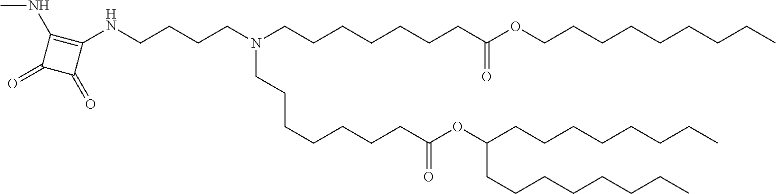 [SMILES: CCCCCCCCCOC(=O)CCCCCCCN(CCCCCCCC(=O)OC(CCCCCCCC)CCCCCCCC)CCCCNc1c(NC)c(=O)c1=O] |
| I-352 | 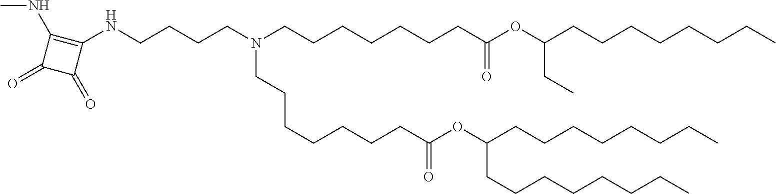 [SMILES: CCCCCCCCC(CC)OC(=O)CCCCCCCN(CCCCCCCC(=O)OC(CCCCCCCC)CCCCCCCC)CCCCNc1c(NC)c(=O)c1=O] |
| I-353 | 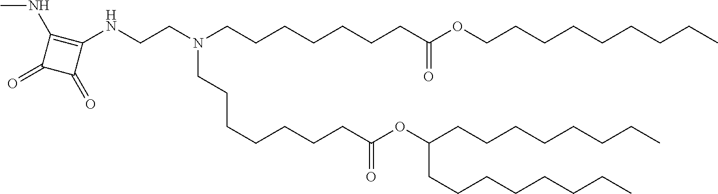 [SMILES: CCCCCCCCCOC(=O)CCCCCCCN(CCCCCCCC(=O)OC(CCCCCCCC)CCCCCCCC)CCNc1c(NC)c(=O)c1=O] |
| I-354 | 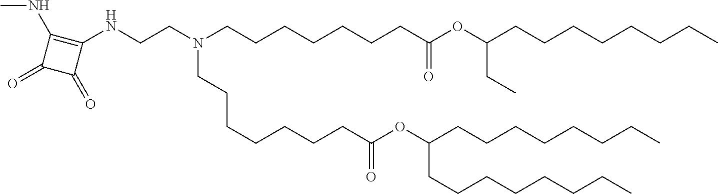 [SMILES: CCCCCCCCC(CC)OC(=O)CCCCCCCN(CCCCCCCC(=O)OC(CCCCCCCC)CCCCCCCC)CCNc1c(NC)c(=O)c1=O] |
| I-355 | 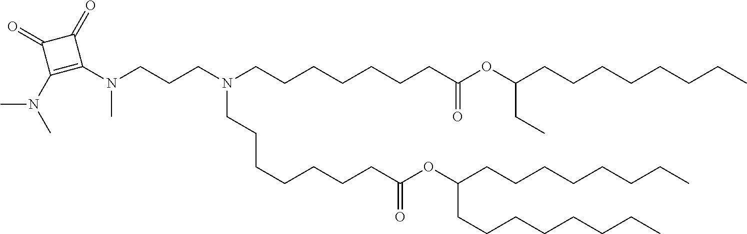 [SMILES: CCCCCCCCC(CC)OC(=O)CCCCCCCN(CCCCCCCC(=O)OC(CCCCCCCC)CCCCCCCC)CCCN(C)c1c(N(C)C)c(=O)c1=O] |
| I-356 | 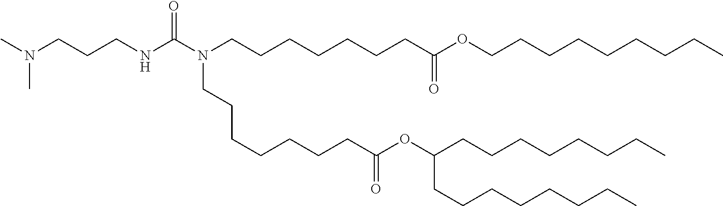 [SMILES: CCCCCCCCCOC(=O)CCCCCCCN(CCCCCCCC(=O)OC(CCCCCCCC)CCCCCCCC)C(=O)NCCCN(C)C] |
| I-357 | 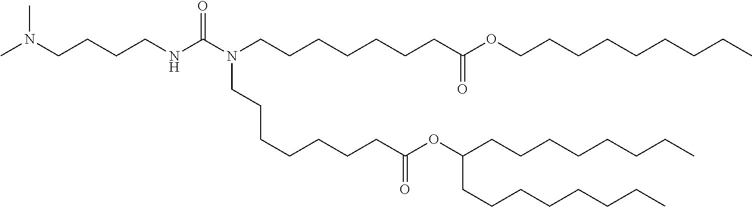 [SMILES: CCCCCCCCCOC(=O)CCCCCCCN(CCCCCCCC(=O)OC(CCCCCCCC)CCCCCCCC)C(=O)NCCCCN(C)C] |
| I-358 | 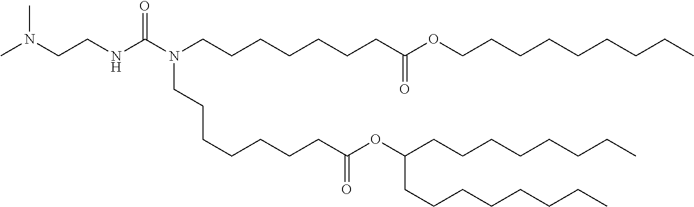 [SMILES: CCCCCCCCCOC(=O)CCCCCCCN(CCCCCCCC(=O)OC(CCCCCCCC)CCCCCCCC)C(=O)NCCN(C)C] |
| I-359 | 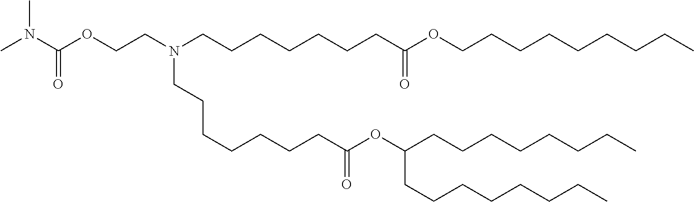 [SMILES: CCCCCCCCCOC(=O)CCCCCCCN(CCCCCCCC(=O)OC(CCCCCCCC)CCCCCCCC)CCOC(=O)N(C)C] |
| I-360 | 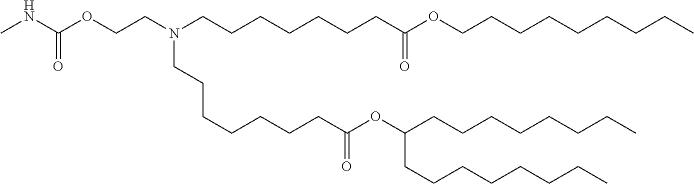 [SMILES: CCCCCCCCCOC(=O)CCCCCCCN(CCCCCCCC(=O)OC(CCCCCCCC)CCCCCCCC)CCOC(=O)NC] |
| I-361 | 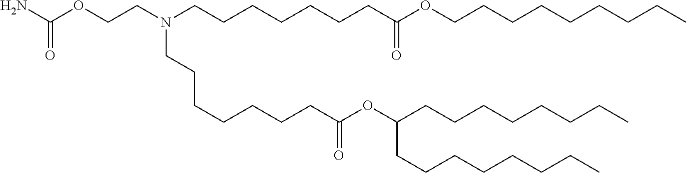 [SMILES: CCCCCCCCCOC(=O)CCCCCCCN(CCCCCCCC(=O)OC(CCCCCCCC)CCCCCCCC)CCOC(N)=O] |
| I-362 | 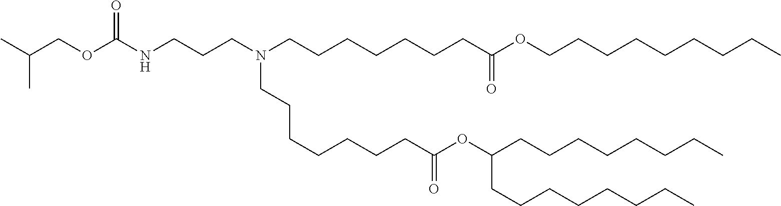 [SMILES: CCCCCCCCCOC(=O)CCCCCCCN(CCCCCCCC(=O)OC(CCCCCCCC)CCCCCCCC)CCCNC(=O)OCC(C)C] |
| I-363 | 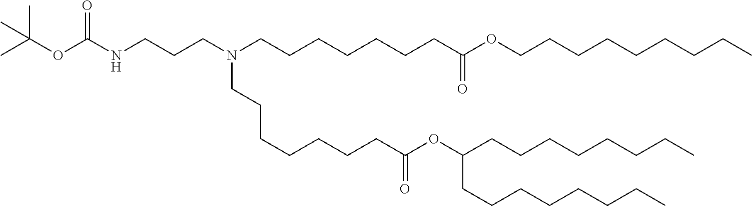 [SMILES: CCCCCCCCCOC(=O)CCCCCCCN(CCCCCCCC(=O)OC(CCCCCCCC)CCCCCCCC)CCCNC(=O)OC(C)(C)C] |
| I-364 | 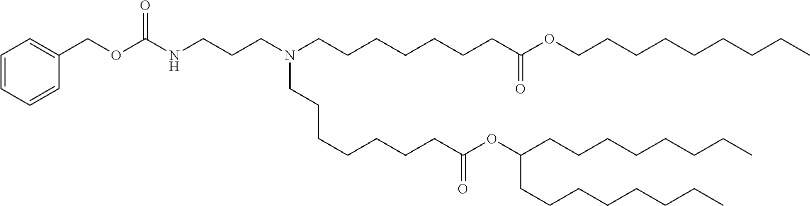 [SMILES: CCCCCCCCCOC(=O)CCCCCCCN(CCCCCCCC(=O)OC(CCCCCCCC)CCCCCCCC)CCCNC(=O)OCc1ccccc1] |
| I-365 | 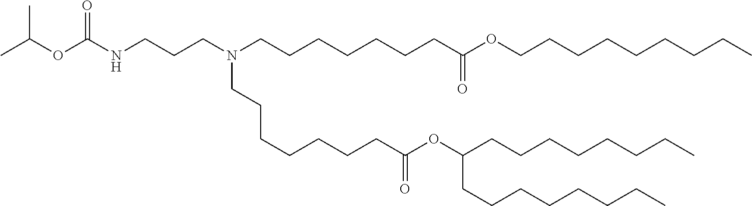 [SMILES: CCCCCCCCCOC(=O)CCCCCCCN(CCCCCCCC(=O)OC(CCCCCCCC)CCCCCCCC)CCCNC(=O)OC(C)C] |
| I-366 | 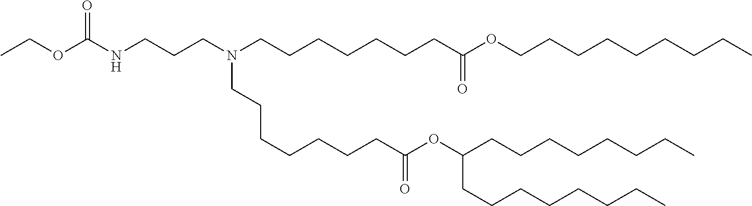 [SMILES: CCCCCCCCCOC(=O)CCCCCCCN(CCCCCCCC(=O)OC(CCCCCCCC)CCCCCCCC)CCCNC(=O)OCC] |
| I-367 | 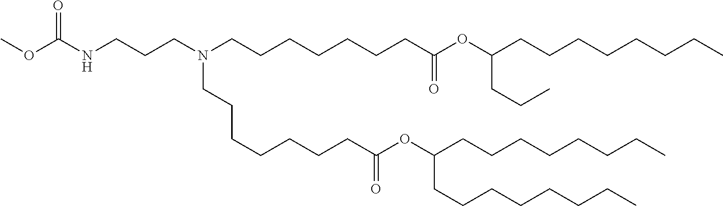 [SMILES: CCCCCCCCC(CCC)OC(=O)CCCCCCCN(CCCCCCCC(=O)OC(CCCCCCCC)CCCCCCCC)CCCNC(=O)OC] |
| I-368 | 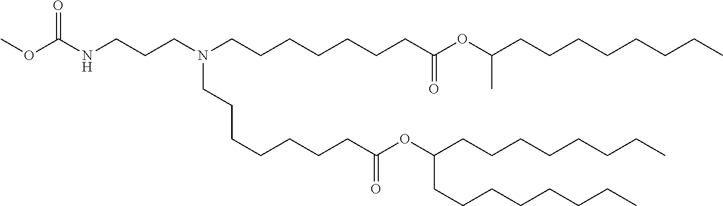 [SMILES: CCCCCCCCC(C)OC(=O)CCCCCCCN(CCCCCCCC(=O)OC(CCCCCCCC)CCCCCCCC)CCCNC(=O)OC] |
| I-369 | 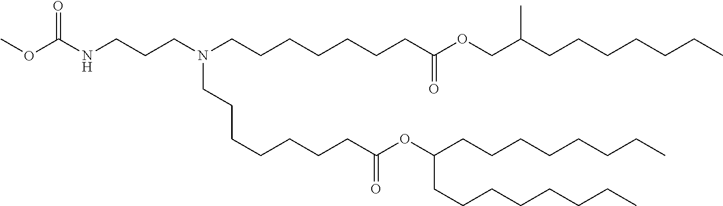 [SMILES: CCCCCCCCC(CCCCCCCC)OC(=O)CCCCCCCN(CCCCCCCC(=O)OCC(C)CCCCCCC)CCCNC(=O)OC] |
| I-370 | 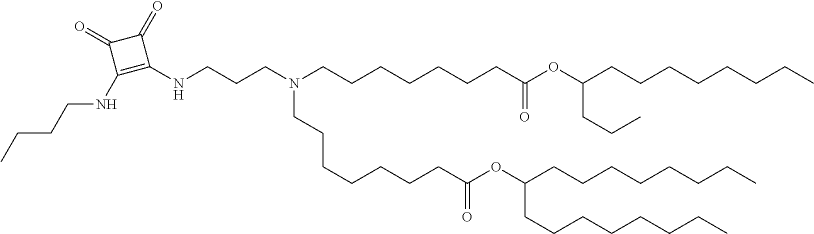 [SMILES: CCCCCCCCC(CCC)OC(=O)CCCCCCCN(CCCCCCCC(=O)OC(CCCCCCCC)CCCCCCCC)CCCNc1c(NCCCC)c(=O)c1=O] |
| I-371 | 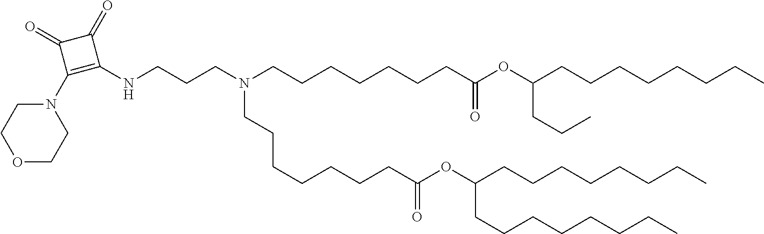 [SMILES: CCCCCCCCC(CCC)OC(=O)CCCCCCCN(CCCCCCCC(=O)OC(CCCCCCCC)CCCCCCCC)CCCNc1c(N2CCOCC2)c(=O)c1=O] |
| I-372 | 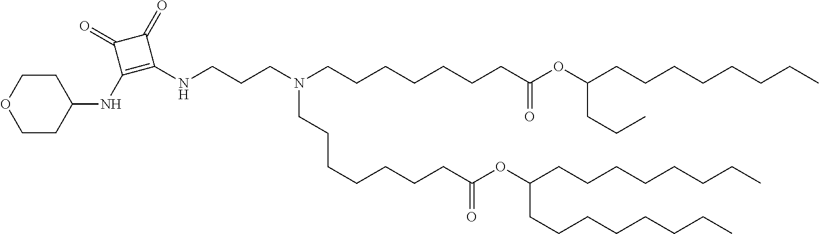 [SMILES: CCCCCCCCC(CCC)OC(=O)CCCCCCCN(CCCCCCCC(=O)OC(CCCCCCCC)CCCCCCCC)CCCNc1c(NC2CCOCC2)c(=O)c1=O] |
| I-373 | 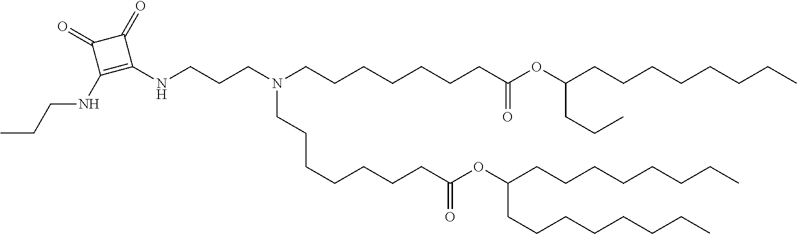 [SMILES: CCCCCCCCC(CCC)OC(=O)CCCCCCCN(CCCCCCCC(=O)OC(CCCCCCCC)CCCCCCCC)CCCNc1c(NCCC)c(=O)c1=O] |
| I-374 | 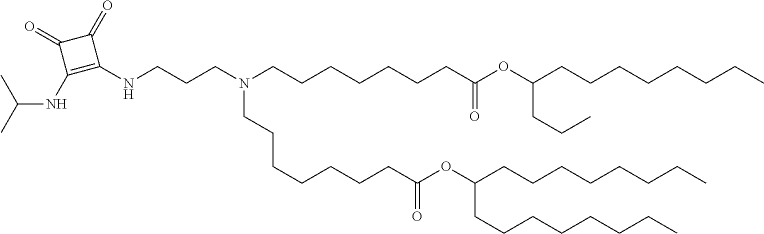 [SMILES: CCCCCCCCC(CCC)OC(=O)CCCCCCCN(CCCCCCCC(=O)OC(CCCCCCCC)CCCCCCCC)CCCNc1c(NC(C)C)c(=O)c1=O] |
| I-375 | 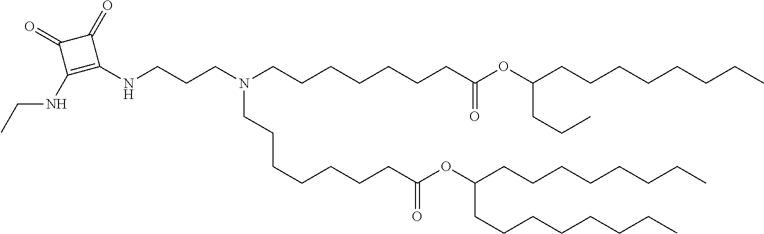 [SMILES: CCCCCCCCC(CCC)OC(=O)CCCCCCCN(CCCCCCCC(=O)OC(CCCCCCCC)CCCCCCCC)CCCNc1c(NCC)c(=O)c1=O] |
| I-376 | 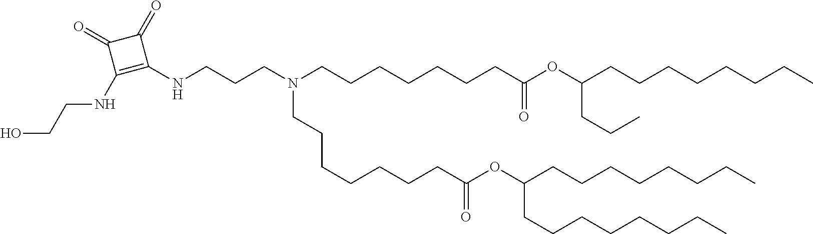 [SMILES: CCCCCCCCC(CCC)OC(=O)CCCCCCCN(CCCCCCCC(=O)OC(CCCCCCCC)CCCCCCCC)CCCNc1c(NCCO)c(=O)c1=O] |
| I-377 | 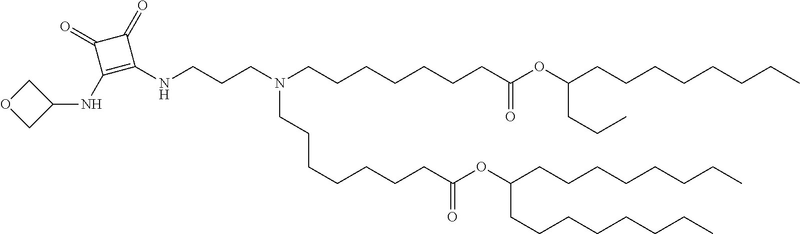 [SMILES: CCCCCCCCC(CCC)OC(=O)CCCCCCCN(CCCCCCCC(=O)OC(CCCCCCCC)CCCCCCCC)CCCNc1c(NC2COC2)c(=O)c1=O] |
| I-378 | 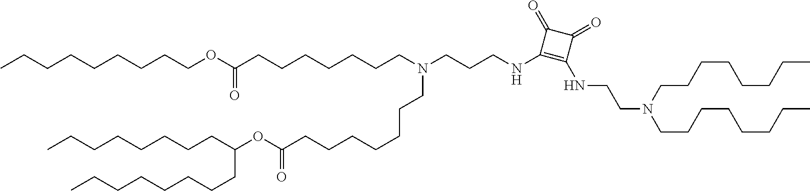 [SMILES: CCCCCCCCCOC(=O)CCCCCCCN(CCCCCCCC(=O)OC(CCCCCCCC)CCCCCCCC)CCCNc1c(NCCN(CCCCCCCC)CCCCCCCC)c(=O)c1=O] |
| I-379 | 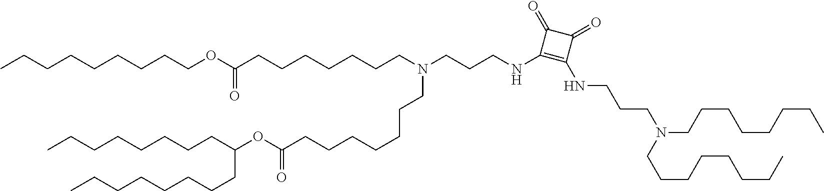 [SMILES: CCCCCCCCCOC(=O)CCCCCCCN(CCCCCCCC(=O)OC(CCCCCCCC)CCCCCCCC)CCCNc1c(NCCCN(CCCCCCCC)CCCCCCCC)c(=O)c1=O] |
| I-380 | 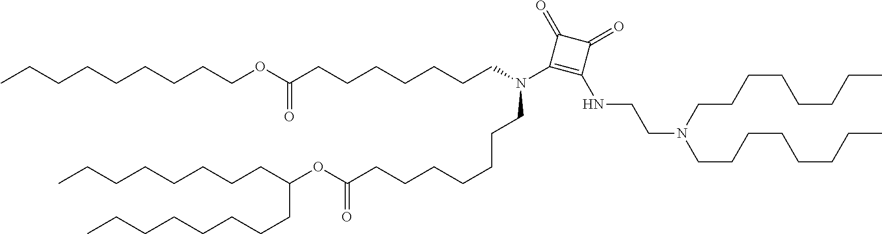 |
| I-381 | 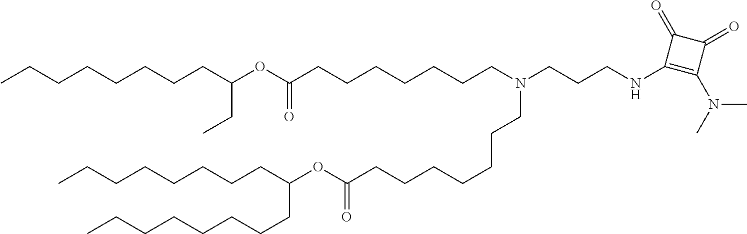 [SMILES: CCCCCCCCC(CC)OC(=O)CCCCCCCN(CCCCCCCC(=O)OC(CCCCCCCC)CCCCCCCC)CCCNc1c(N(C)C)c(=O)c1=O] |
| I-382 | 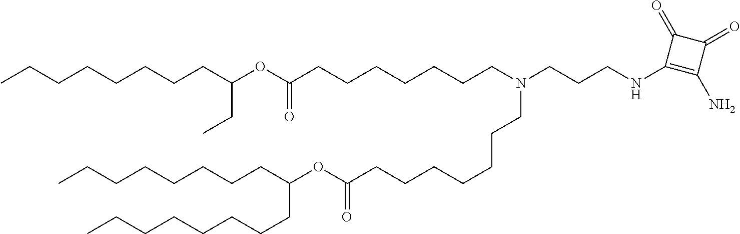 [SMILES: CCCCCCCCC(CC)OC(=O)CCCCCCCN(CCCCCCCC(=O)OC(CCCCCCCC)CCCCCCCC)CCCNc1c(N)c(=O)c1=O] |
| I-383 | 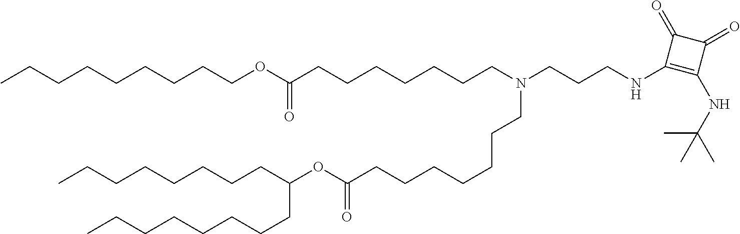 [SMILES: CCCCCCCCCOC(=O)CCCCCCCN(CCCCCCCC(=O)OC(CCCCCCCC)CCCCCCCC)CCCNc1c(NC(C)(C)C)c(=O)c1=O] |
| I-384 | 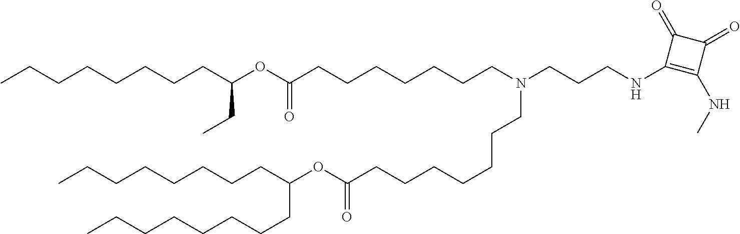 [SMILES: CCCCCCCCC(CCCCCCCC)OC(=O)CCCCCCCN(CCCCCCCC(=O)O[C@@H](CC)CCCCCCCC)CCCNc1c(NC)c(=O)c1=O] |
| I-385 | 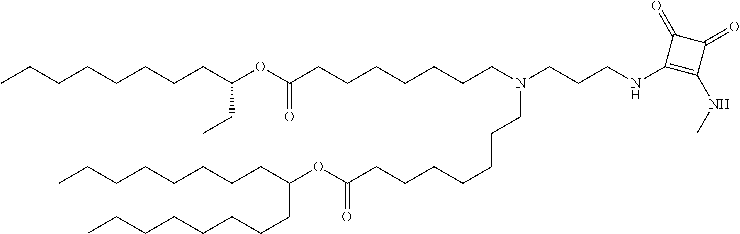 [SMILES: CCCCCCCCC(CCCCCCCC)OC(=O)CCCCCCCN(CCCCCCCC(=O)O[C@H](CC)CCCCCCCC)CCCNc1c(NC)c(=O)c1=O] |
| I-386 | 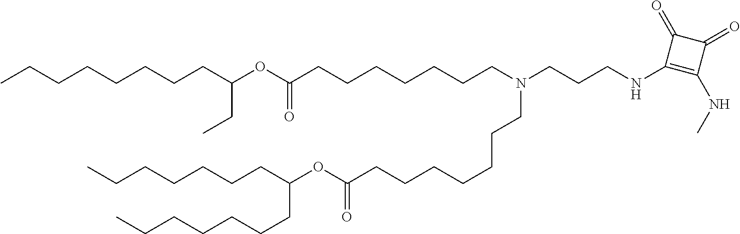 [SMILES: CCCCCCCCC(CC)OC(=O)CCCCCCCN(CCCCCCCC(=O)OC(CCCCCCC)CCCCCCC)CCCNc1c(NC)c(=O)c1=O] |
| I-387 | 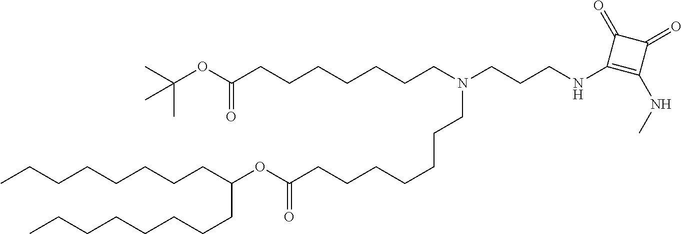 [SMILES: CCCCCCCCC(CCCCCCCC)OC(=O)CCCCCCCN(CCCCCCCC(=O)OC(C)(C)C)CCCNc1c(NC)c(=O)c1=O] |
| I-388 | 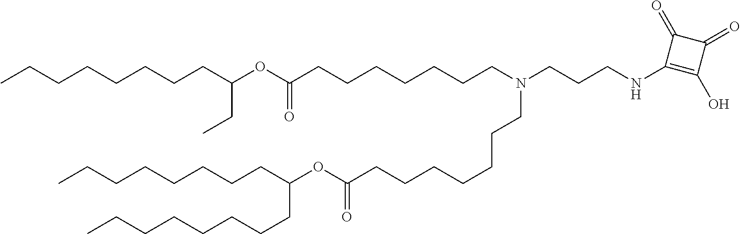 [SMILES: CCCCCCCCC(CC)OC(=O)CCCCCCCN(CCCCCCCC(=O)OC(CCCCCCCC)CCCCCCCC)CCCNc1c(O)c(=O)c1=O] |
| I-389 | 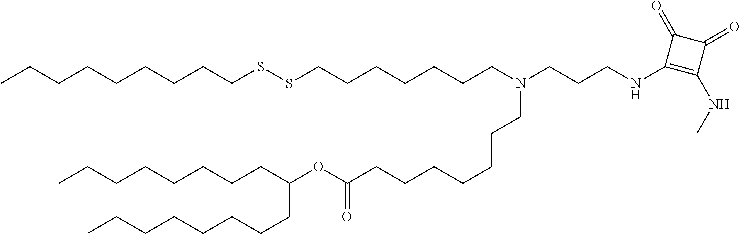 [SMILES: CCCCCCCCCSSCCCCCCCN(CCCCCCCC(=O)OC(CCCCCCCC)CCCCCCCC)CCCNc1c(NC)c(=O)c1=O] |
| I-390 | 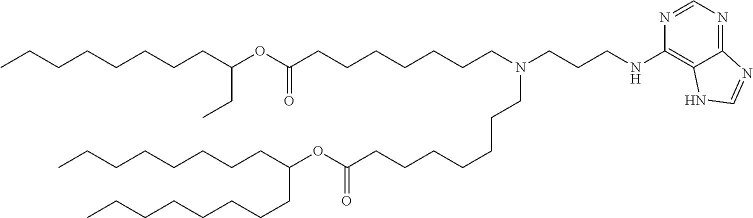 [SMILES: CCCCCCCCC(CC)OC(=O)CCCCCCCN(CCCCCCCC(=O)OC(CCCCCCCC)CCCCCCCC)CCCNc1ncnc2nc[nH]c12] |
| I-391 | 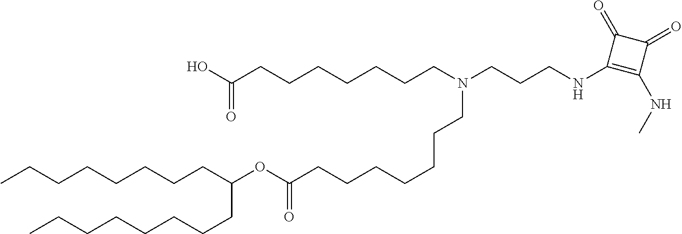 [SMILES: CCCCCCCCC(CCCCCCCC)OC(=O)CCCCCCCN(CCCCCCCC(=O)O)CCCNc1c(NC)c(=O)c1=O] |
| I-392 | 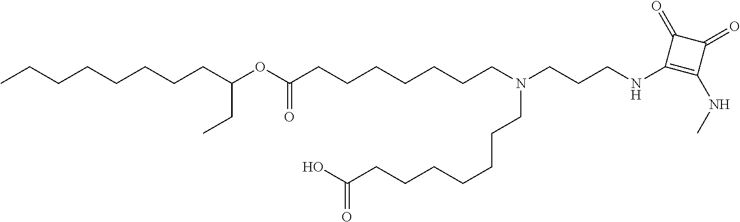 [SMILES: CCCCCCCCC(CC)OC(=O)CCCCCCCN(CCCCCCCC(=O)O)CCCNc1c(NC)c(=O)c1=O] |

In some embodiments, the ionizable lipid is a compound selected from Table IL-2.

TABLE IL-2
Ionizable lipids

| Cpd | |
|---|---|
| # | Structure |
| II-1 | 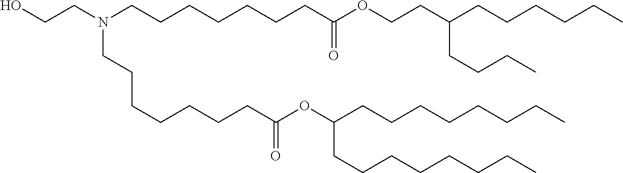 [SMILES: CCCCCCCCC(CCCCCCCC)OC(=O)CCCCCCCN(CCO)CCCCCCCC(=O)OCCC(CCCC)CCCCCC] |
| II-2 | 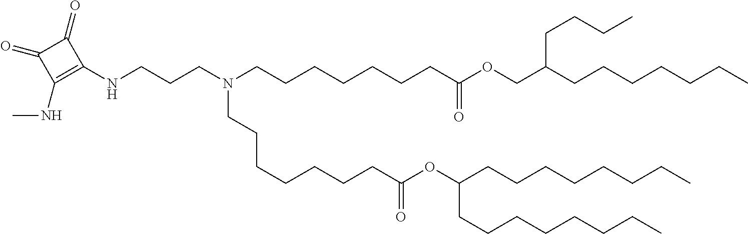 [SMILES: CCCCCCCCC(CCCCCCCC)OC(=O)CCCCCCCN(CCCCCCCC(=O)OCC(CCCC)CCCCCCC)CCCNc1c(NC)c(=O)c1=O] |
| II-3 | 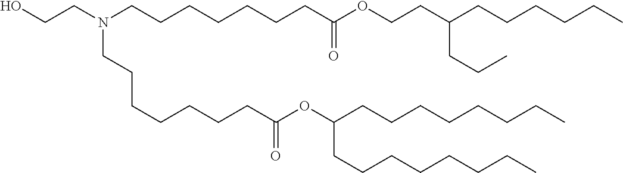 [SMILES: CCCCCCCCC(CCCCCCCC)OC(=O)CCCCCCCN(CCO)CCCCCCCC(=O)OCCC(CCC)CCCCCC] |
| II-4 | 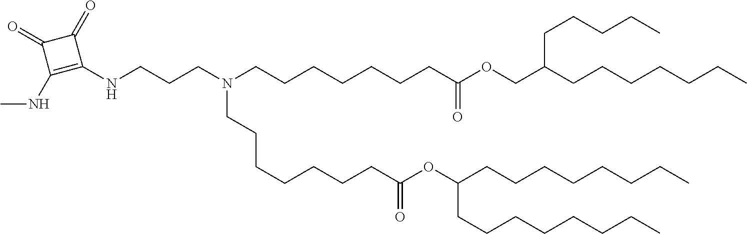 [SMILES: CCCCCCCCC(CCCCCCCC)OC(=O)CCCCCCCN(CCCCCCCC(=O)OCC(CCCCC)CCCCCCC)CCCNc1c(NC)c(=O)c1=O] |
| II-5 | 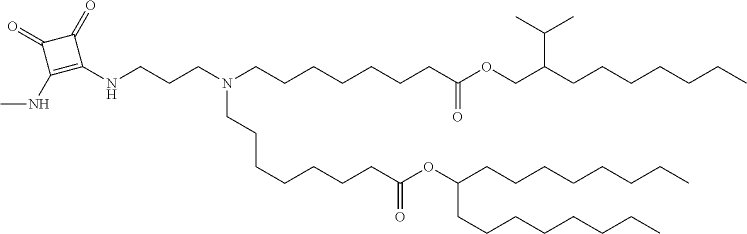 [SMILES: CCCCCCCCC(CCCCCCCC)OC(=O)CCCCCCCN(CCCCCCCC(=O)OCC(CCCCCCC)C(C)C)CCCNc1c(NC)c(=O)c1=O] |
| II-6 | 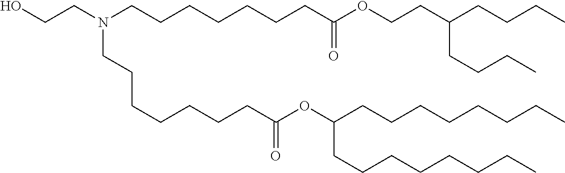 [SMILES: CCCCCCCCC(CCCCCCCC)OC(=O)CCCCCCCN(CCO)CCCCCCCC(=O)OCCC(CCCC)CCCC] |
| II-7 | 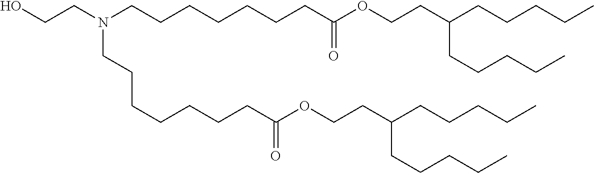 [SMILES: CCCCCC(CCCCC)CCOC(=O)CCCCCCCN(CCO)CCCCCCCC(=O)OCCC(CCCCC)CCCCC] |
| II-8 | 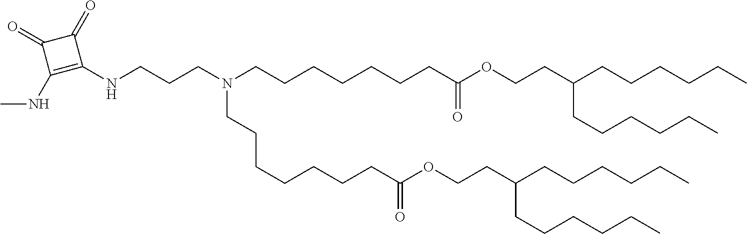 [SMILES: CCCCCCC(CCCCCC)CCOC(=O)CCCCCCCN(CCCCCCCC(=O)OCCC(CCCCCC)CCCCCC)CCCNc1c(NC)c(=O)c1=O] |
| II-9 | 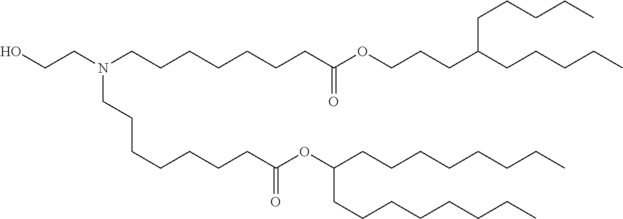 [SMILES: CCCCCCCCC(CCCCCCCC)OC(=O)CCCCCCCN(CCO)CCCCCCCC(=O)OCCCC(CCCCC)CCCCC] |
| II-10 | 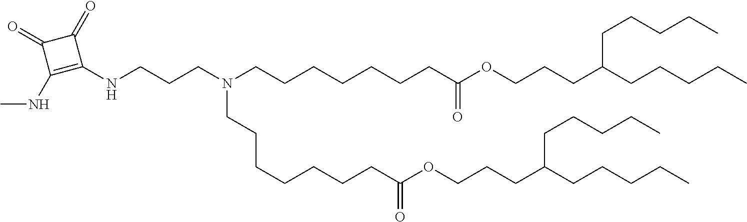 [SMILES: CCCCCC(CCCCC)CCCOC(=O)CCCCCCCN(CCCCCCCC(=O)OCCCC(CCCCC)CCCCC)CCCNc1c(NC)c(=O)c1=O] |
| II-11 | 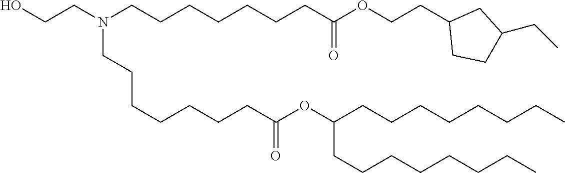 [SMILES: CCCCCCCCC(CCCCCCCC)OC(=O)CCCCCCCN(CCO)CCCCCCCC(=O)OCCC1CCC(CC)C1] |
| II-12 | 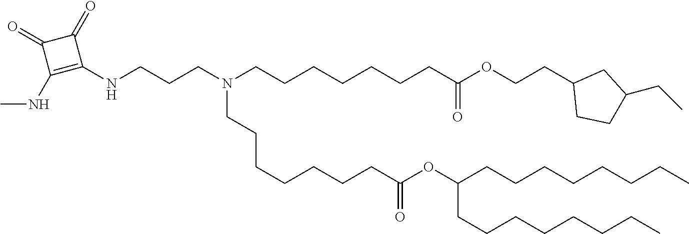 [SMILES: CCCCCCCCC(CCCCCCCC)OC(=O)CCCCCCCN(CCCCCCCC(=O)OCCC1CCC(CC)C1)CCCNc1c(NC)c(=O)c1=O] |
| II-13 | 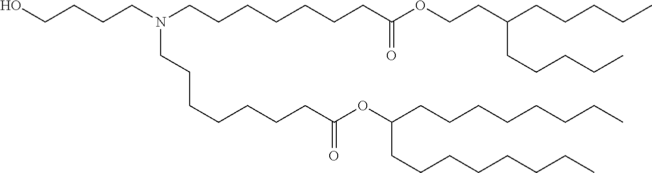 [SMILES: CCCCCCCCC(CCCCCCCC)OC(=O)CCCCCCCN(CCCCO)CCCCCCCC(=O)OCCC(CCCCC)CCCCC] |
| II-14 | 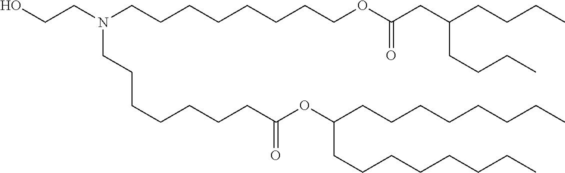 [SMILES: CCCCCCCCC(CCCCCCCC)OC(=O)CCCCCCCN(CCO)CCCCCCCCOC(=O)CC(CCCC)CCCC] |
| II-15 | 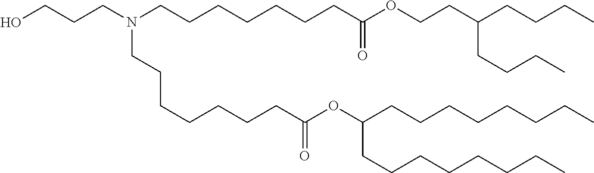 [SMILES: CCCCCCCCC(CCCCCCCC)OC(=O)CCCCCCCN(CCCO)CCCCCCCC(=O)OCCC(CCCC)CCCC] |
| II-16 | 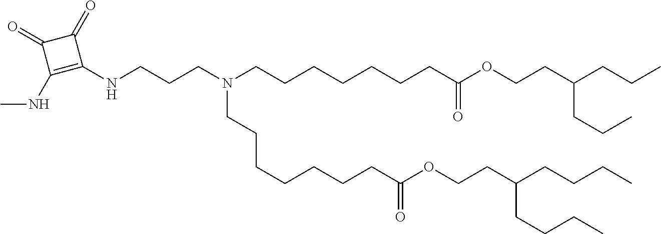 [SMILES: CCCCC(CCCC)CCOC(=O)CCCCCCCN(CCCCCCCC(=O)OCCC(CCC)CCC)CCCNc1c(NC)c(=O)c1=O] |
| II-17 | 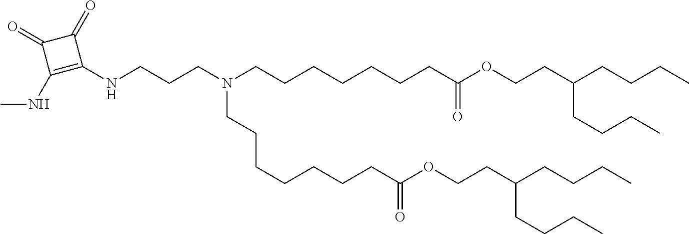 [SMILES: CCCCC(CCCC)CCOC(=O)CCCCCCCN(CCCCCCCC(=O)OCCC(CCCC)CCCC)CCCNc1c(NC)c(=O)c1=O] |
| II-18 | 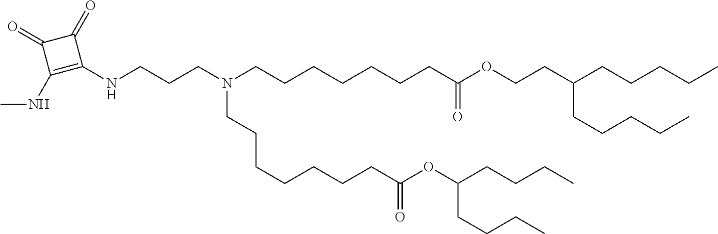 [SMILES: CCCCCC(CCCCC)CCOC(=O)CCCCCCCN(CCCCCCCC(=O)OC(CCCC)CCCC)CCCNc1c(NC)c(=O)c1=O] |
| II-19 | 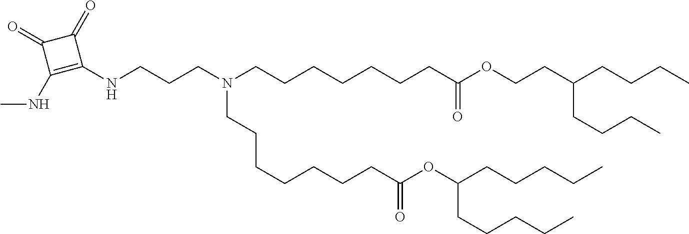 [SMILES: CCCCCC(CCCCC)OC(=O)CCCCCCCN(CCCCCCCC(=O)OCCC(CCCC)CCCC)CCCNc1c(NC)c(=O)c1=O] |
| II-20 | 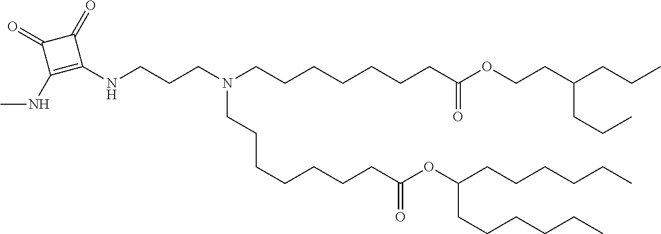 [SMILES: CCCCCCC(CCCCCC)OC(=O)CCCCCCCN(CCCCCCCC(=O)OCCC(CCC)CCC)CCCNc1c(NC)c(=O)c1=O] |
| II-21 | 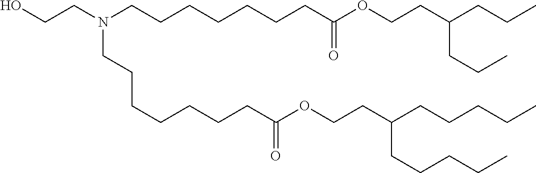 [SMILES: CCCCCC(CCCCC)CCOC(=O)CCCCCCCN(CCO)CCCCCCCC(=O)OCCC(CCC)CCC] |
| II-22 | 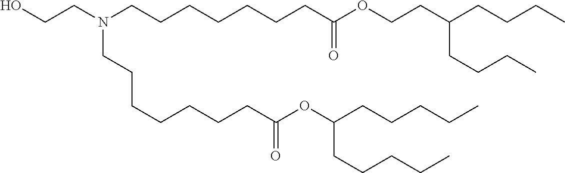 [SMILES: CCCCCC(CCCCC)OC(=O)CCCCCCCN(CCO)CCCCCCCC(=O)OCCC(CCCC)CCCC] |
| II-23 | 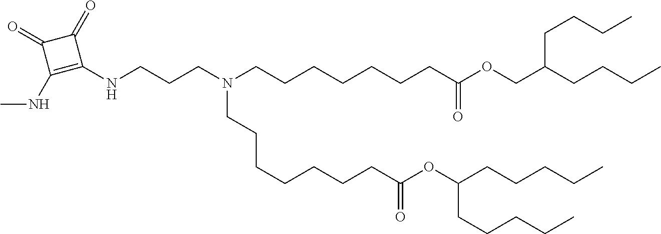 [SMILES: CCCCCC(CCCCC)OC(=O)CCCCCCCN(CCCCCCCC(=O)OCC(CCCC)CCCC)CCCNc1c(NC)c(=O)c1=O] |
| II-24 | 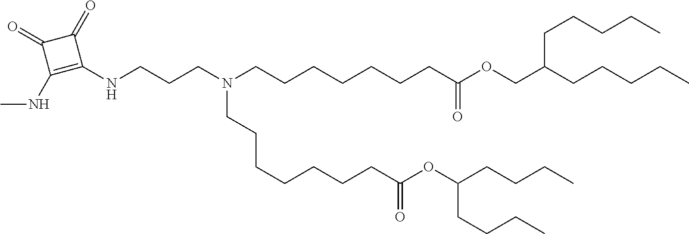 [SMILES: CCCCCC(CCCCC)COC(=O)CCCCCCCN(CCCCCCCC(=O)OC(CCCC)CCCC)CCCNc1c(NC)c(=O)c1=O] |
| II-25 | 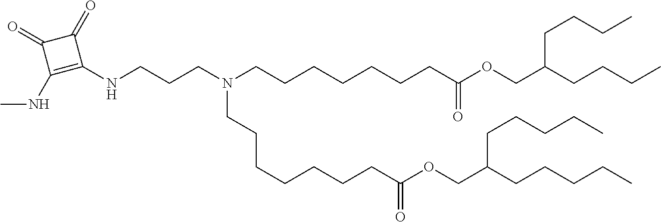 [SMILES: CCCCCC(CCCCC)COC(=O)CCCCCCCN(CCCCCCCC(=O)OCC(CCCC)CCCC)CCCNc1c(NC)c(=O)c1=O] |
| II-26 | 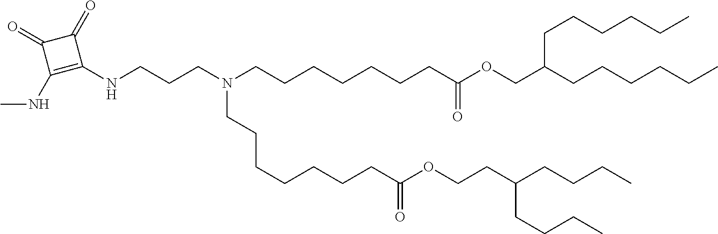 [SMILES: CCCCCCC(CCCCCC)COC(=O)CCCCCCCN(CCCCCCCC(=O)OCCC(CCCC)CCCC)CCCNc1c(NC)c(=O)c1=O] |
| II-27 | 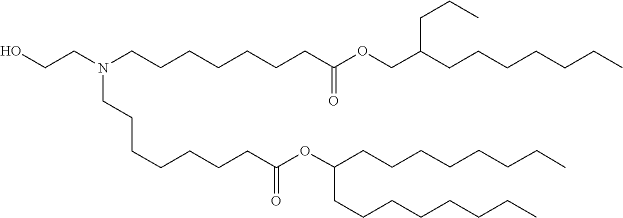 [SMILES: CCCCCCCCC(CCCCCCCC)OC(=O)CCCCCCCN(CCO)CCCCCCCC(=O)OCC(CCC)CCCCCCC] |
| II-28 | 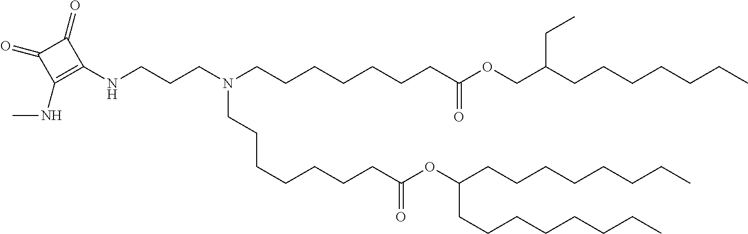 [SMILES: CCCCCCCCC(CCCCCCCC)OC(=O)CCCCCCCN(CCCCCCCC(=O)OCC(CC)CCCCCCC)CCCNc1c(NC)c(=O)c1=O] |
| II-29 | 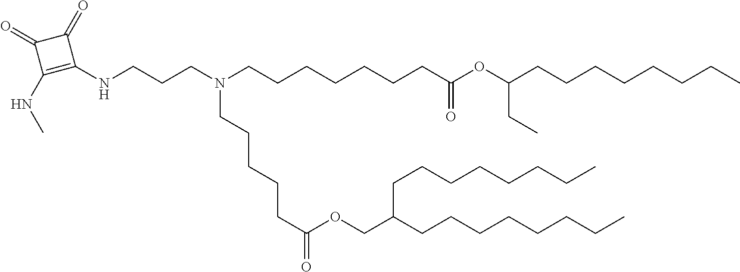 [SMILES: CCCCCCCCC(CCCCCCCC)COC(=O)CCCCCN(CCCCCCCC(=O)OC(CC)CCCCCCCC)CCCNc1c(NC)c(=O)c1=O] |
| II-30 | 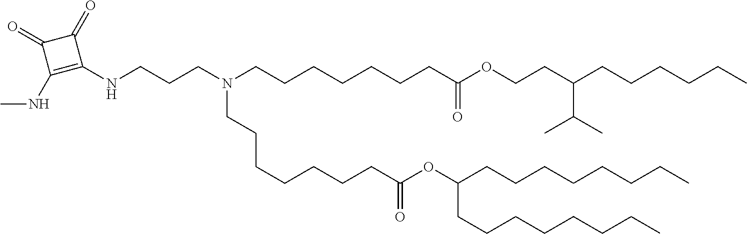 [SMILES: CCCCCCCCC(CCCCCCCC)OC(=O)CCCCCCCN(CCCCCCCC(=O)OCCC(CCCCCC)C(C)C)CCCNc1c(NC)c(=O)c1=O] |
| II-31 | 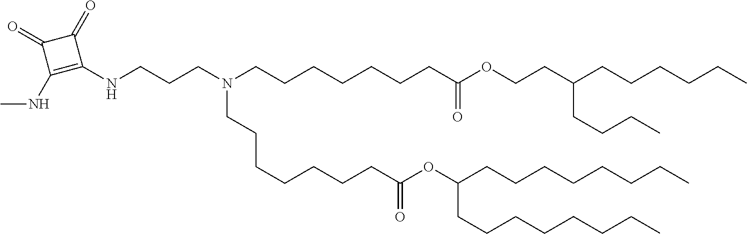 [SMILES: CCCCCCCCC(CCCCCCCC)OC(=O)CCCCCCCN(CCCCCCCC(=O)OCCC(CCCC)CCCCCC)CCCNc1c(NC)c(=O)c1=O] |
| II-32 | 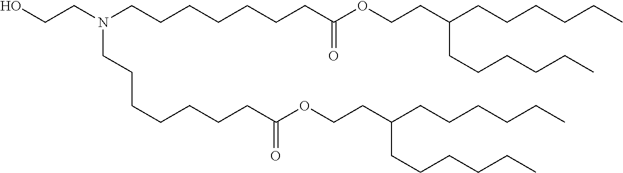 [SMILES: CCCCCCC(CCCCCC)CCOC(=O)CCCCCCCN(CCO)CCCCCCCC(=O)OCCC(CCCCCC)CCCCCC] |
| II-33 | 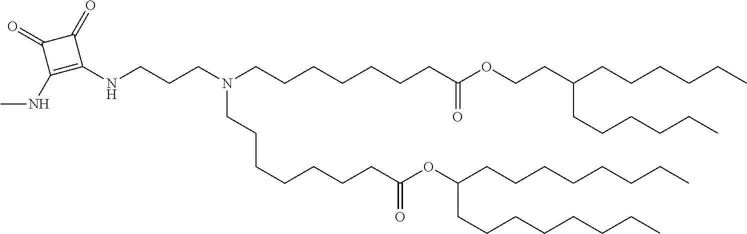 [SMILES: CCCCCCCCC(CCCCCCCC)OC(=O)CCCCCCCN(CCCCCCCC(=O)OCCC(CCCCCC)CCCCCC)CCCNc1c(NC)c(=O)c1=O] |
| II-34 | 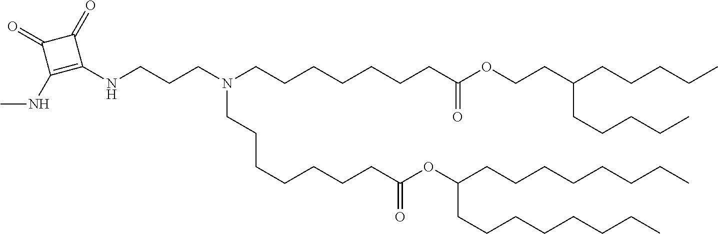 [SMILES: CCCCCCCCC(CCCCCCCC)OC(=O)CCCCCCCN(CCCCCCCC(=O)OCCC(CCCCC)CCCCC)CCCNc1c(NC)c(=O)c1=O] |
| II-35 | 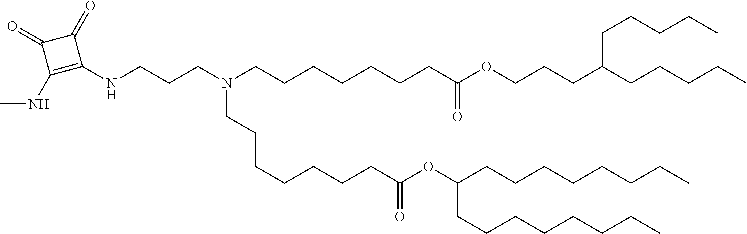 [SMILES: CCCCCCCCC(CCCCCCCC)OC(=O)CCCCCCCN(CCCCCCCC(=O)OCCCC(CCCCC)CCCCC)CCCNc1c(NC)c(=O)c1=O] |
| II-36 | 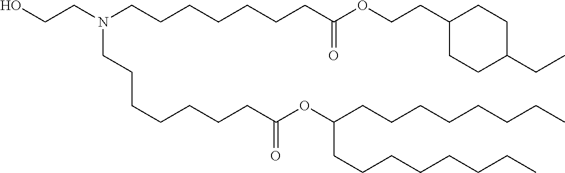 [SMILES: CCCCCCCCC(CCCCCCCC)OC(=O)CCCCCCCN(CCO)CCCCCCCC(=O)OCCC1CCC(CC)CC1] |
| II-37 | 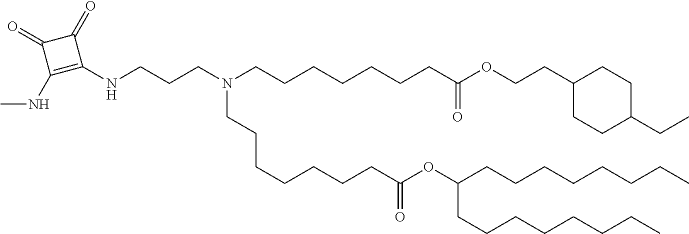 [SMILES: CCCCCCCCC(CCCCCCCC)OC(=O)CCCCCCCN(CCCCCCCC(=O)OCCC1CCC(CC)CC1)CCCNc1c(NC)c(=O)c1=O] |
| II-38 | 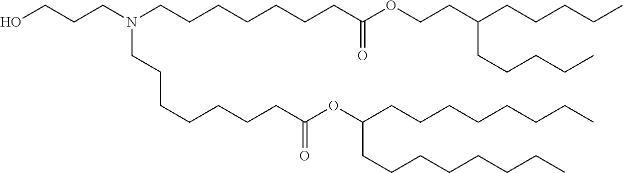 [SMILES: CCCCCCCCC(CCCCCCCC)OC(=O)CCCCCCCN(CCCO)CCCCCCCC(=O)OCCC(CCCCC)CCCCC] |
| II-39 | 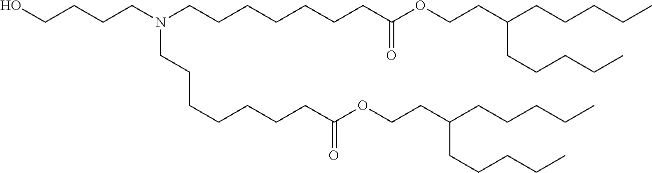 [SMILES: CCCCCC(CCCCC)CCOC(=O)CCCCCCCN(CCCCO)CCCCCCCC(=O)OCCC(CCCCC)CCCCC] |
| II-40 | 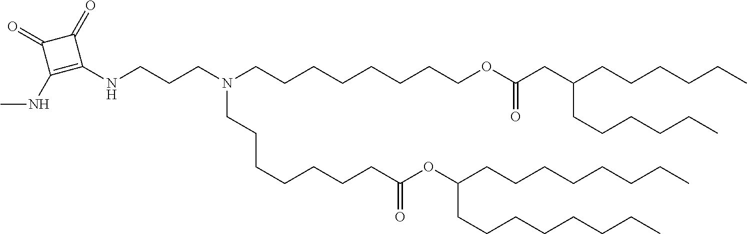 [SMILES: CCCCCCCCC(CCCCCCCC)OC(=O)CCCCCCCN(CCCCCCCCOC(=O)CC(CCCCCC)CCCCCC)CCCNc1c(NC)c(=O)c1=O] |
| II-41 | 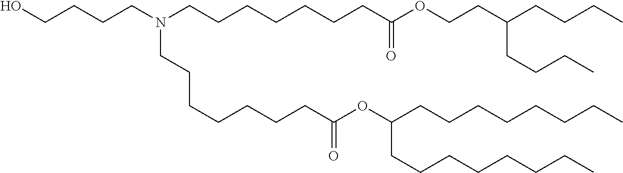 [SMILES: CCCCCCCCC(CCCCCCCC)OC(=O)CCCCCCCN(CCCCO)CCCCCCCC(=O)OCCC(CCCC)CCCC] |
| II-42 | 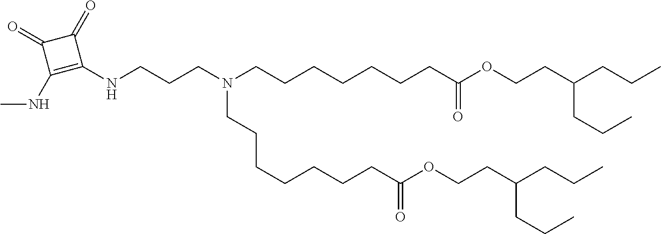 [SMILES: CCCC(CCC)CCOC(=O)CCCCCCCN(CCCCCCCC(=O)OCCC(CCC)CCC)CCCNc1c(NC)c(=O)c1=O] |
| II-43 | 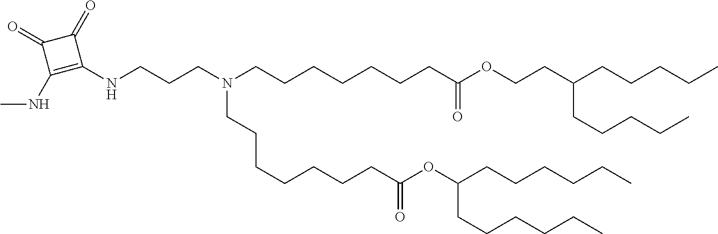 [SMILES: CCCCCCC(CCCCCC)OC(=O)CCCCCCCN(CCCCCCCC(=O)OCCC(CCCCC)CCCCC)CCCNc1c(NC)c(=O)c1=O] |
| II-44 | 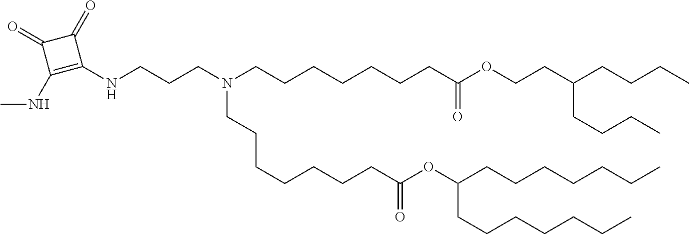 [SMILES: CCCCCCCC(CCCCCCC)OC(=O)CCCCCCCN(CCCCCCCC(=O)OCCC(CCCC)CCCC)CCCNc1c(NC)c(=O)c1=O] |
| II-45 | 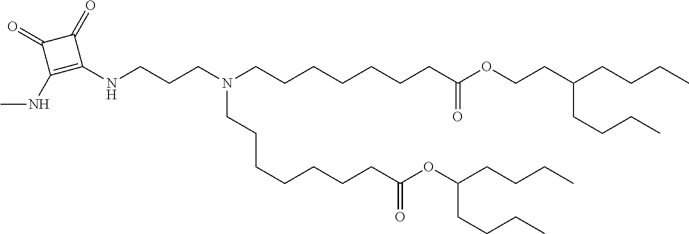 [SMILES: CCCCC(CCCC)CCOC(=O)CCCCCCCN(CCCCCCCC(=O)OC(CCCC)CCCC)CCCNc1c(NC)c(=O)c1=O] |
| II-46 | 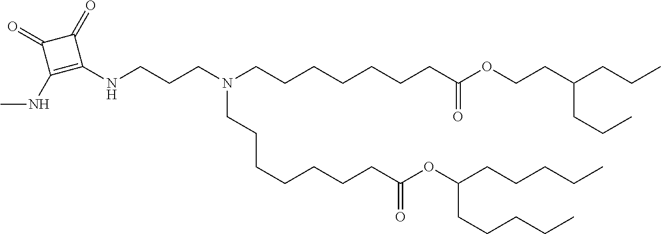 [SMILES: CCCCCC(CCCCC)OC(=O)CCCCCCCN(CCCCCCCC(=O)OCCC(CCC)CCC)CCCNc1c(NC)c(=O)c1=O] |
| II-47 | 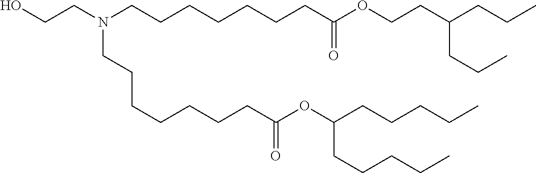 [SMILES: CCCCCC(CCCCC)OC(=O)CCCCCCCN(CCO)CCCCCCCC(=O)OCCC(CCC)CCC] |
| II-48 | 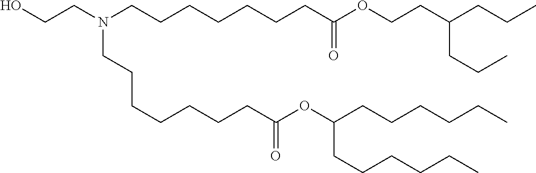 [SMILES: CCCCCCC(CCCCCC)OC(=O)CCCCCCCN(CCO)CCCCCCCC(=O)OCCC(CCC)CCC] |
| II-50 | 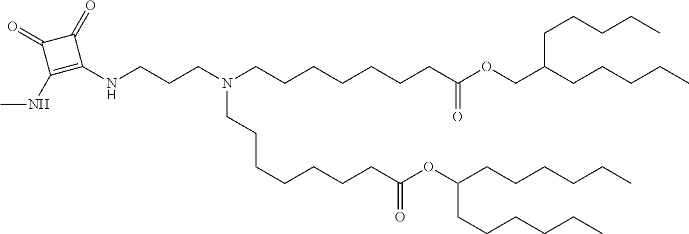 [SMILES: CCCCCCC(CCCCCC)OC(=O)CCCCCCCN(CCCCCCCC(=O)OCC(CCCCC)CCCCC)CCCNc1c(NC)c(=O)c1=O] |
| II-51 | 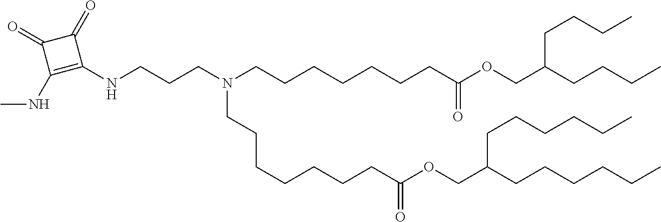 [SMILES: CCCCCCC(CCCCCC)COC(=O)CCCCCCCN(CCCCCCCC(=O)OCC(CCCC)CCCC)CCCNc1c(NC)c(=O)c1=O] |
| II-52 | 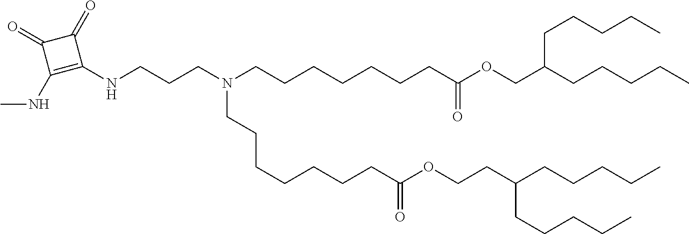 [SMILES: CCCCCC(CCCCC)CCOC(=O)CCCCCCCN(CCCCCCCC(=O)OCC(CCCCC)CCCCC)CCCNc1c(NC)c(=O)c1=O] |
| II-53 | 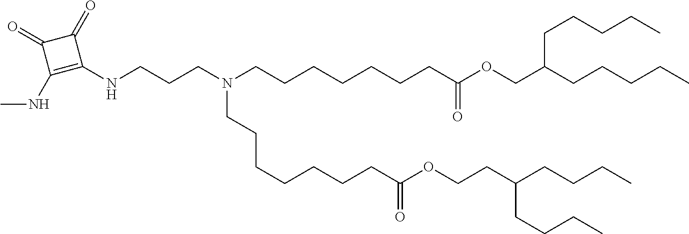 [SMILES: CCCCCC(CCCCC)COC(=O)CCCCCCCN(CCCCCCCC(=O)OCCC(CCCC)CCCC)CCCNc1c(NC)c(=O)c1=O] |
| II-54 | 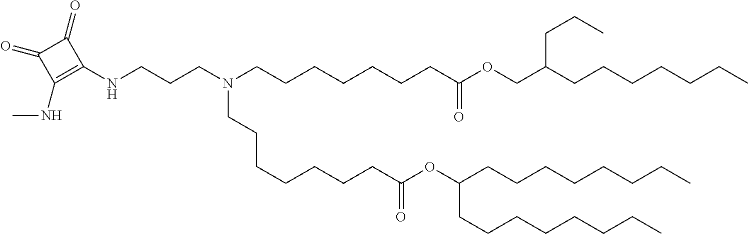 [SMILES: CCCCCCCCC(CCCCCCCC)OC(=O)CCCCCCCN(CCCCCCCC(=O)OCC(CCC)CCCCCCC)CCCNc1c(NC)c(=O)c1=O] |
| II-55 | 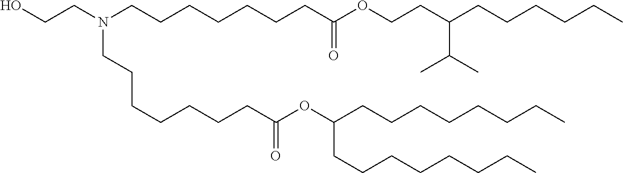 [SMILES: CCCCCCCCC(CCCCCCCC)OC(=O)CCCCCCCN(CCO)CCCCCCCC(=O)OCCC(CCCCCC)C(C)C] |
| II-56 | 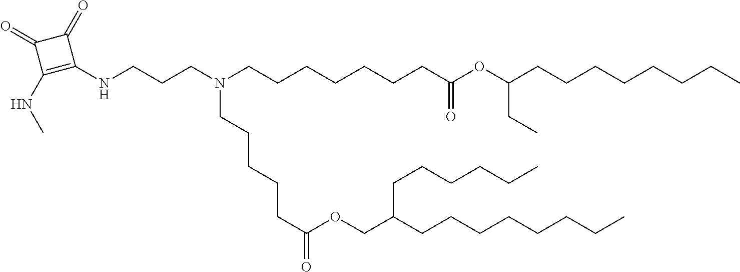 [SMILES: CCCCCCCCC(CCCCCC)COC(=O)CCCCCN(CCCCCCCC(=O)OC(CC)CCCCCCCC)CCCNc1c(NC)c(=O)c1=O] |
| II-57 | 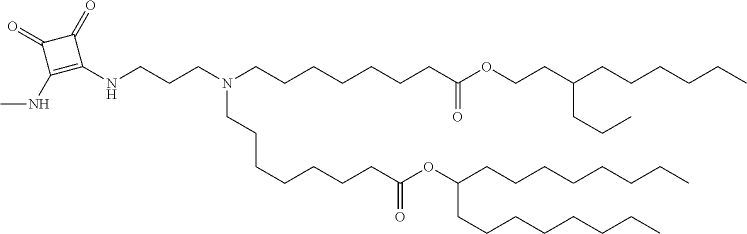 [SMILES: CCCCCCCCC(CCCCCCCC)OC(=O)CCCCCCCN(CCCCCCCC(=O)OCCC(CCC)CCCCCC)CCCNc1c(NC)c(=O)c1=O] |
| II-58 | 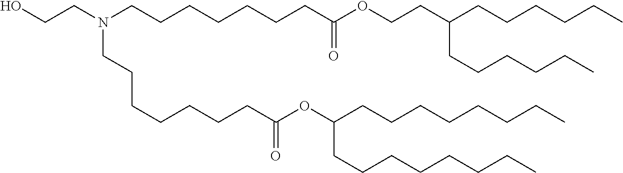 [SMILES: CCCCCCCCC(CCCCCCCC)OC(=O)CCCCCCCN(CCO)CCCCCCCC(=O)OCCC(CCCCCC)CCCCCC] |
| II-59 | 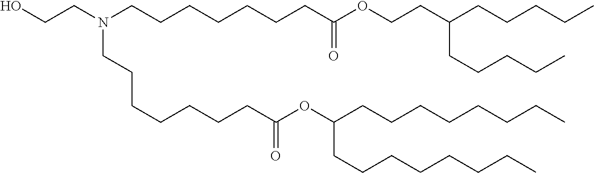 [SMILES: CCCCCCCCC(CCCCCCCC)OC(=O)CCCCCCCN(CCO)CCCCCCCC(=O)OCCC(CCCCC)CCCCC] |
| II-60 | 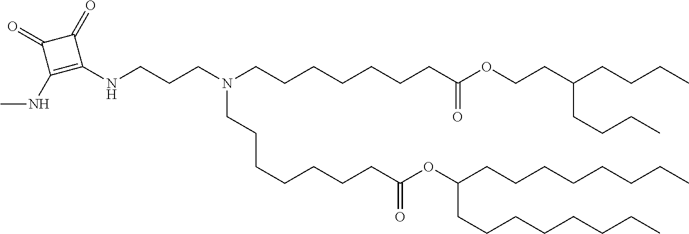 [SMILES: CCCCCCCCC(CCCCCCCC)OC(=O)CCCCCCCN(CCCCCCCC(=O)OCCC(CCCC)CCCC)CCCNc1c(NC)c(=O)c1=O] |
| II-61 | 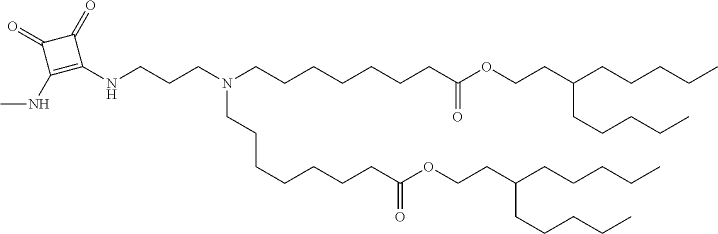 [SMILES: CCCCCC(CCCCC)CCOC(=O)CCCCCCCN(CCCCCCCC(=O)OCCC(CCCCC)CCCCC)CCCNc1c(NC)c(=O)c1=O] |
| II-62 | 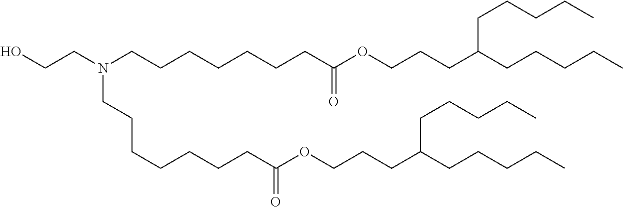 [SMILES: CCCCCC(CCCCC)CCCOC(=O)CCCCCCCN(CCO)CCCCCCCC(=O)OCCCC(CCCCC)CCCCC] |
| II-63 | 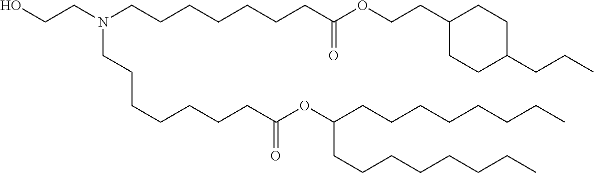 [SMILES: CCCCCCCCC(CCCCCCCC)OC(=O)CCCCCCCN(CCO)CCCCCCCC(=O)OCCC1CCC(CCC)CC1] |
| II-64 | 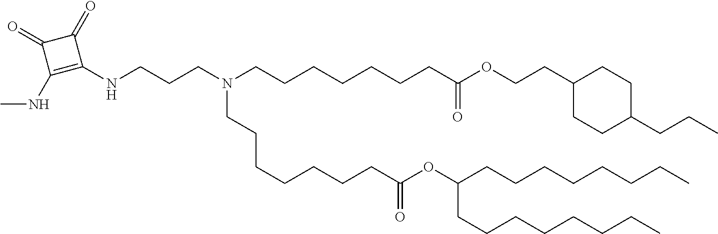 [SMILES: CCCCCCCCC(CCCCCCCC)OC(=O)CCCCCCCN(CCCCCCCC(=O)OCCC1CCC(CCC)CC1)CCCNc1c(NC)c(=O)c1=O] |
| II-65 | 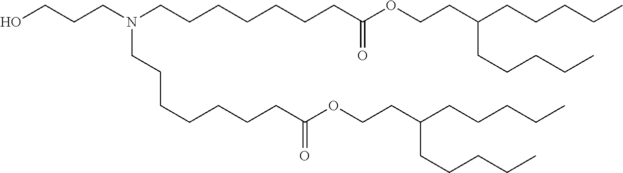 [SMILES: CCCCCC(CCCCC)CCOC(=O)CCCCCCCN(CCCO)CCCCCCCC(=O)OCCC(CCCCC)CCCCC] |
| II-66 | 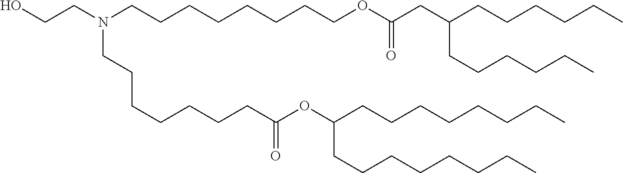 [SMILES: CCCCCCCCC(CCCCCCCC)OC(=O)CCCCCCCN(CCO)CCCCCCCCOC(=O)CC(CCCCCC)CCCCCC] |
| II-67 | 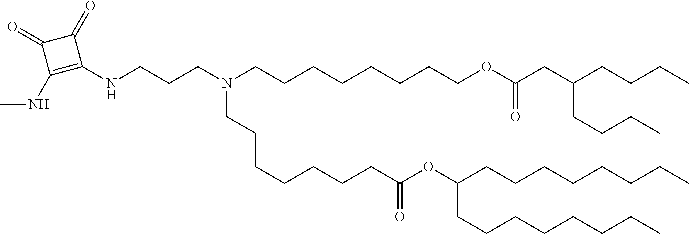 [SMILES: CCCCCCCCC(CCCCCCCC)OC(=O)CCCCCCCN(CCCCCCCCOC(=O)CC(CCCC)CCCC)CCCNc1c(NC)c(=O)c1=O] |
| II-68 | 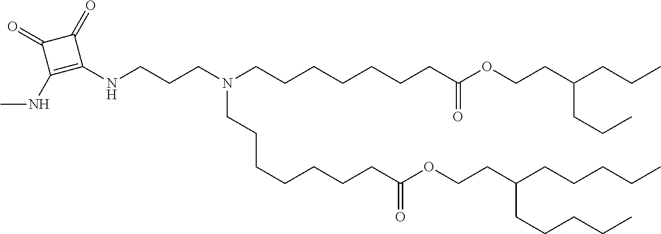 [SMILES: CCCCCC(CCCCC)CCOC(=O)CCCCCCCN(CCCCCCCC(=O)OCCC(CCC)CCC)CCCNc1c(NC)c(=O)c1=O] |
| II-68 | 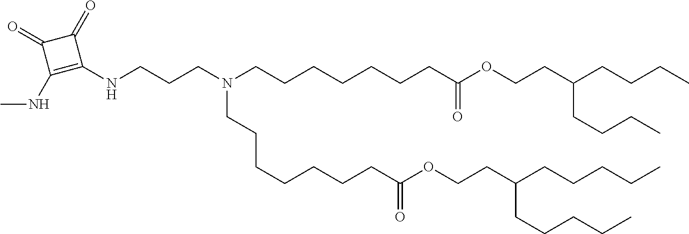 [SMILES: CCCCCC(CCCCC)CCOC(=O)CCCCCCCN(CCCCCCCC(=O)OCCC(CCCC)CCCC)CCCNc1c(NC)c(=O)c1=O] |
| II-69 | 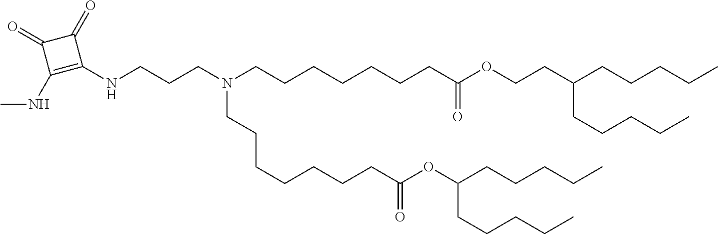 [SMILES: CCCCCC(CCCCC)CCOC(=O)CCCCCCCN(CCCCCCCC(=O)OC(CCCCC)CCCCC)CCCNc1c(NC)c(=O)c1=O] |
| II-70 | 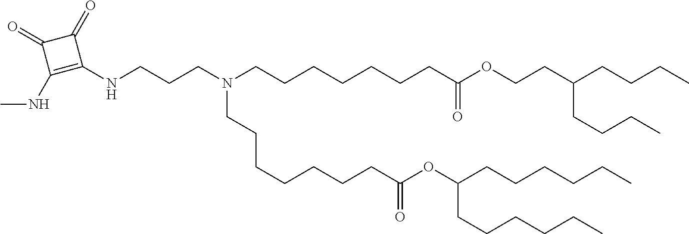 [SMILES: CCCCCCC(CCCCCC)OC(=O)CCCCCCCN(CCCCCCCC(=O)OCCC(CCCC)CCCC)CCCNc1c(NC)c(=O)c1=O] |
| II-71 | 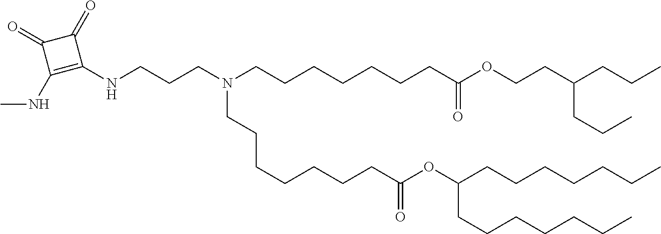 [SMILES: CCCCCCCC(CCCCCCC)OC(=O)CCCCCCCN(CCCCCCCC(=O)OCCC(CCC)CCC)CCCNc1c(NC)c(=O)c1=O] |
| II-72 | 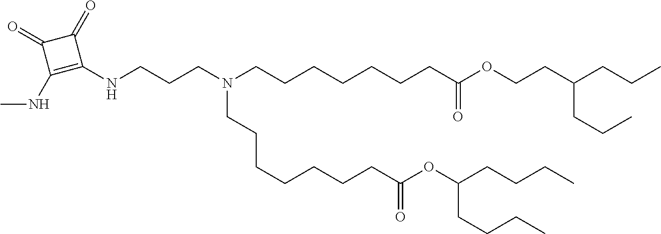 [SMILES: CCCCC(CCCC)OC(=O)CCCCCCCN(CCCCCCCC(=O)OCCC(CCC)CCC)CCCNc1c(NC)c(=O)c1=O] |
| II-73 | 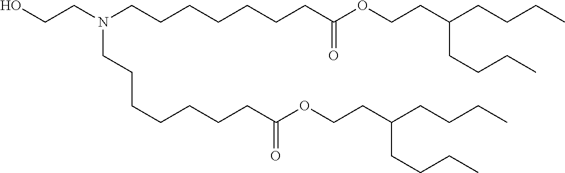 [SMILES: CCCCC(CCCC)CCOC(=O)CCCCCCCN(CCO)CCCCCCCC(=O)OCCC(CCCC)CCCC] |
| II-74 | 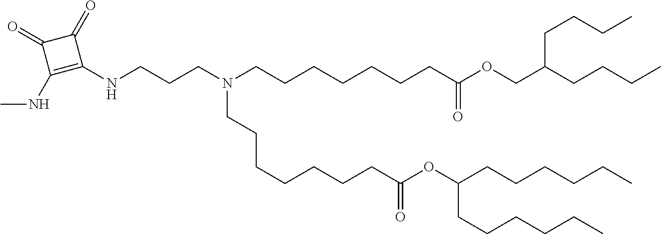 [SMILES: CCCCCCC(CCCCCC)OC(=O)CCCCCCCN(CCCCCCCC(=O)OCC(CCCC)CCCC)CCCNc1c(NC)c(=O)c1=O] |
| II-75 | 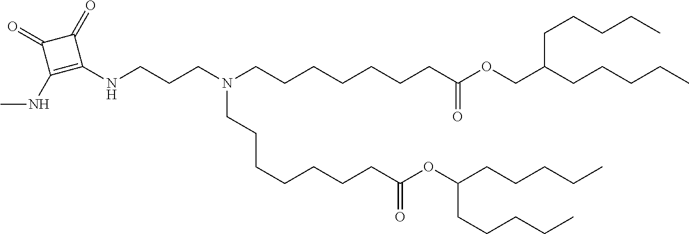 [SMILES: CCCCCC(CCCCC)COC(=O)CCCCCCCN(CCCCCCCC(=O)OC(CCCCC)CCCCC)CCCNc1c(NC)c(=O)c1=O] |
| II-76 | 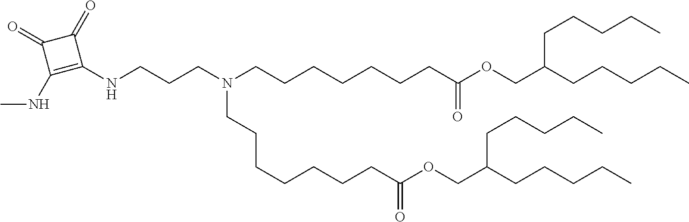 [SMILES: CCCCCC(CCCCC)COC(=O)CCCCCCCN(CCCCCCCC(=O)OCC(CCCCC)CCCCC)CCCNc1c(NC)c(=O)c1=O] |
| II-77 | 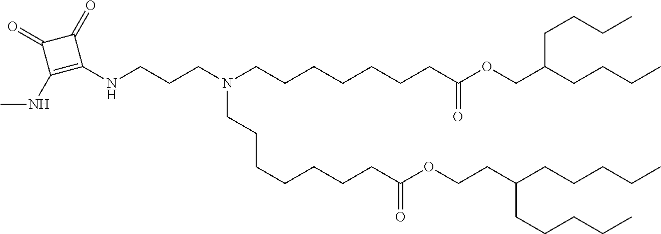 [SMILES: CCCCCC(CCCCC)CCOC(=O)CCCCCCCN(CCCCCCCC(=O)OCC(CCCC)CCCC)CCCNc1c(NC)c(=O)c1=O] |
| II-78 | 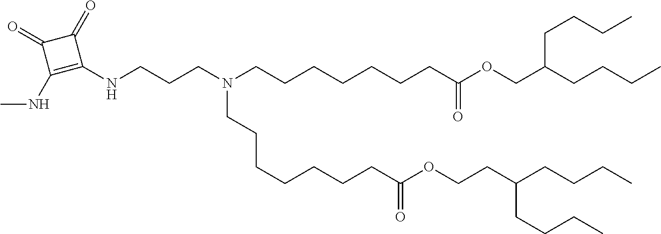 [SMILES: CCCCC(CCCC)CCOC(=O)CCCCCCCN(CCCCCCCC(=O)OCC(CCCC)CCCC)CCCNc1c(NC)c(=O)c1=O] |

In some aspects the ionizable lipid is a compound of Formula (IL-IIA):

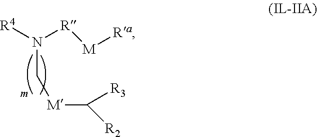

or its N-oxide, or a salt or isomer thereof, wherein:
m is selected from 5, 6, 7, 8, and 9;
R² and R³ are each independently selected from the group consisting of H, C₁₋₁₄ alkyl, and C₂₋₁₄ alkenyl;
R⁴ is selected from —(CH₂)ₙOH, wherein n is selected from 1, 2, 3, 4, and 5, and

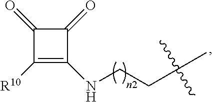

wherein n2 is selected from 1, 2, 3, 4, 5, 6, 7, 8, 9, and 10; and
R¹⁰ is —N(R)₂, wherein each R is independently selected from the group consisting of C₁₋₆ alkyl, C₂₋₃ alkenyl, and H;
M is selected from —OC(O)O—, —C(O)O—, —O-M″-O—, and —N(R^{M})C(O)—, in which M″ is —(CH₂)_{z}C(O)—, wherein z is 1, 2, 3, or 4;
M′ is selected from —OC(O)O—, —C(O)O—, —O-M″-O—, —N(R^{M})C(O)O—, and —O—N═C(R^{M})—, wherein:
   M″ is —(CH₂)_{z}C(O)—, C₁₋₁₃ alkyl, —B(R**)—, or —Si(R**)₂—;
   z is 1, 2, 3, or 4;
   each R^{M} is independently selected from H and C₁₋₆ alkyl;
   each R** is independently selected from H and C₁₋₁₂ alkyl;
R′^{a} is C₁₋₁₈ alkyl, C₂₋₁₈ alkenyl, or —R*YR*″, wherein:
   each R*″ is independently C₁₋₁₅ alkyl;
   each R* is independently C₁₋₁₂ alkyl;
   each Y is independently a C₃₋₆ carbocycle; and
R″ is a C₃-C₁₃ alkyl, optionally substituted with OH.

In some aspects, the ionizable lipid is a compound of Formula (IL-IIAX):

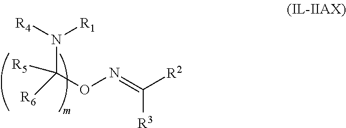

or its N-oxide, or a salt or isomer thereof, wherein:
R¹ is —R″M′R′, wherein:
   each R′ is independently C₁₋₁₈ alkyl;
   M′ is selected from —C(O)O— and —O—N═C(R^{M})—, wherein each R^{M} is independently selected from H and C₁₋₆ alkyl;
   each R″ is independently C₃₋₁₅ alkyl;
R² and R³ are each independently selected from the group consisting of H, C₁₋₁₄ alkyl, and C₂₋₁₄ alkenyl;
R⁴ is selected from —(CH₂)ₙOH, wherein n is selected from 1, 2, 3, 4, and 5, and

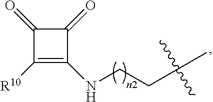

wherein n2 is selected from 1, 2, 3, 4, 5, 6, 7, 8, 9, and 10; and
R¹⁰ is —N(R)₂, wherein each R is independently selected from the group consisting of C₁₋₆ alkyl, C₂₋₃ alkenyl, and H;
each R⁵ is H;
each R⁶ is H; and
m is selected from 5, 6, 7, 8, 9, 10, 11, 12, and 13.

In some embodiments, the ionizable lipid is a compound selected from Table IL-3.

TABLE IL-3
Ionizable lipids

| Cpd | |
|---|---|
| # | Structure |
| III-1 | 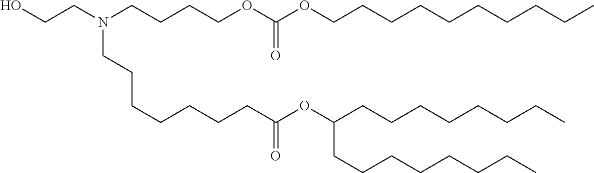 [SMILES: CCCCCCCCCCOC(=O)OCCCCN(CCO)CCCCCCCC(=O)OC(CCCCCCCC)CCCCCCCC] |
| III-2 | 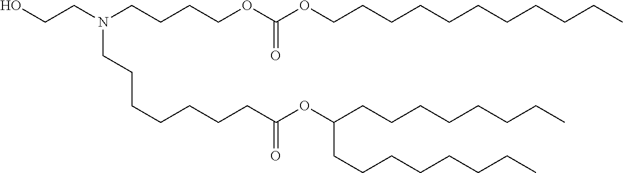 [SMILES: CCCCCCCCCCCOC(=O)OCCCCN(CCO)CCCCCCCC(=O)OC(CCCCCCCC)CCCCCCCC] |
| III-3 | 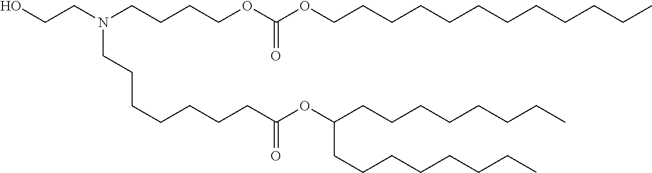 [SMILES: CCCCCCCCCCCCOC(=O)OCCCCN(CCO)CCCCCCCC(=O)OC(CCCCCCCC)CCCCCCCC] |
| III-4 | 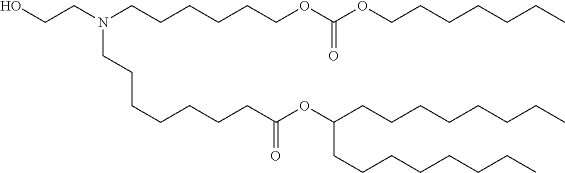 [SMILES: CCCCCCCCC(CCCCCCCC)OC(=O)CCCCCCCN(CCO)CCCCCCOC(=O)OCCCCCCC] |
| III-5 | 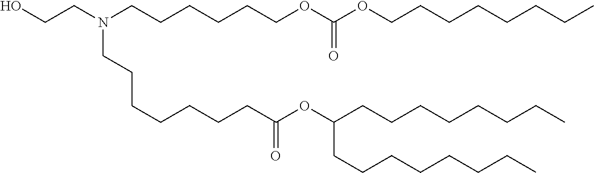 [SMILES: CCCCCCCCOC(=O)OCCCCCCN(CCO)CCCCCCCC(=O)OC(CCCCCCCC)CCCCCCCC] |
| III-6 | 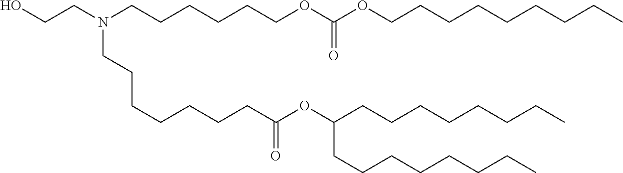 [SMILES: CCCCCCCCCOC(=O)OCCCCCCN(CCO)CCCCCCCC(=O)OC(CCCCCCCC)CCCCCCCC] |
| III-7 | 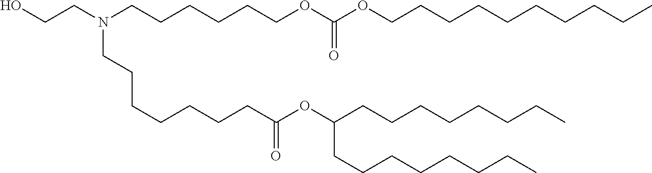 [SMILES: CCCCCCCCCCOC(=O)OCCCCCCN(CCO)CCCCCCCC(=O)OC(CCCCCCCC)CCCCCCCC] |
| III-8 | 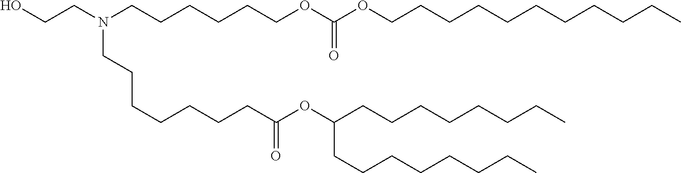 [SMILES: CCCCCCCCCCCOC(=O)OCCCCCCN(CCO)CCCCCCCC(=O)OC(CCCCCCCC)CCCCCCCC] |
| III-9 | 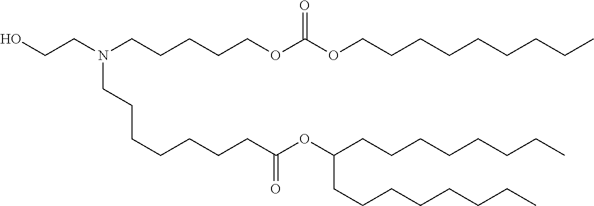 [SMILES: CCCCCCCCCOC(=O)OCCCCCN(CCO)CCCCCCCC(=O)OC(CCCCCCCC)CCCCCCCC] |
| III-10 | 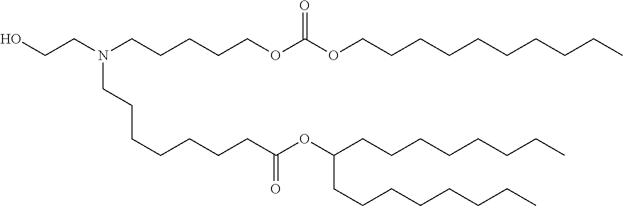 [SMILES: CCCCCCCCCCOC(=O)OCCCCCN(CCO)CCCCCCCC(=O)OC(CCCCCCCC)CCCCCCCC] |
| III-11 | 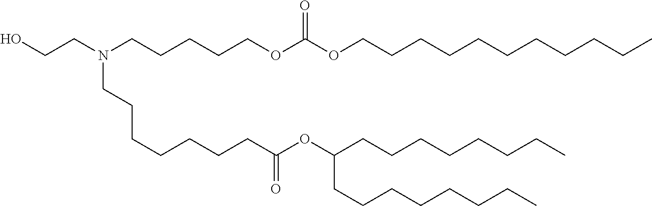 [SMILES: CCCCCCCCCCCOC(=O)OCCCCCN(CCO)CCCCCCCC(=O)OC(CCCCCCCC)CCCCCCCC] |
| III-12 | 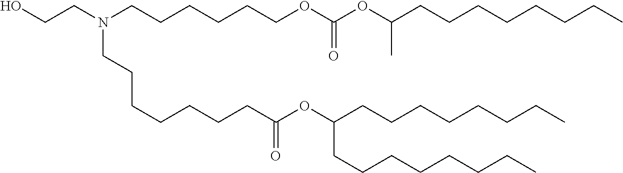 [SMILES: CCCCCCCCC(C)OC(=O)OCCCCCCN(CCO)CCCCCCCC(=O)OC(CCCCCCCC)CCCCCCCC] |
| III-13 | 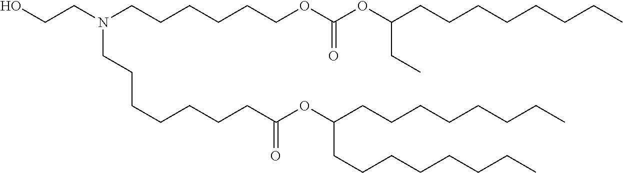 [SMILES: CCCCCCCCC(CCCCCCCC)OC(=O)CCCCCCCN(CCO)CCCCCCOC(=O)OC(CC)CCCCCCCC] |
| III-14 | 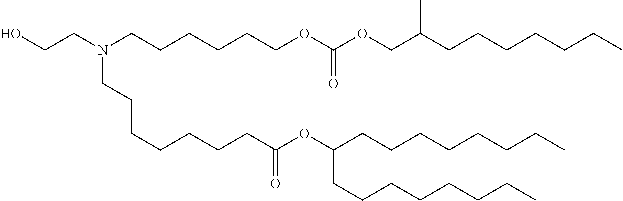 [SMILES: CCCCCCCCC(CCCCCCCC)OC(=O)CCCCCCCN(CCO)CCCCCCOC(=O)OCC(C)CCCCCCC] |
| III-15 | 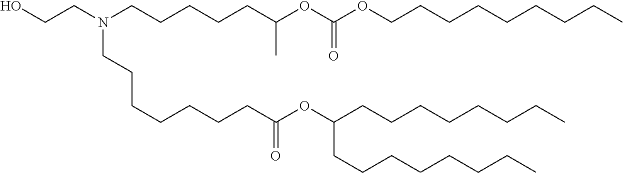 [SMILES: CCCCCCCCCOC(=O)OC(C)CCCCCN(CCO)CCCCCCCC(=O)OC(CCCCCCCC)CCCCCCCC] |
| III-16 | 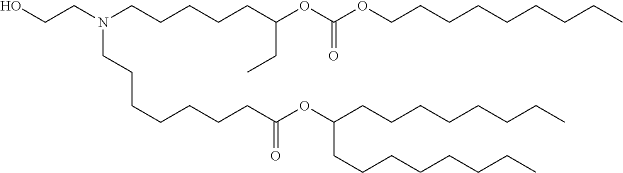 [SMILES: CCCCCCCCCOC(=O)OC(CC)CCCCCN(CCO)CCCCCCCC(=O)OC(CCCCCCCC)CCCCCCCC] |
| III-17 | 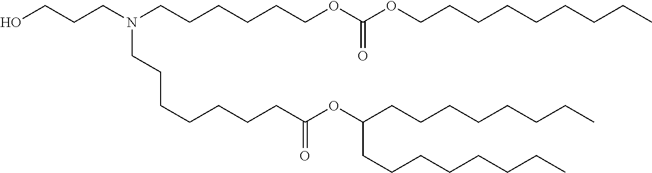 [SMILES: CCCCCCCCCOC(=O)OCCCCCCN(CCCO)CCCCCCCC(=O)OC(CCCCCCCC)CCCCCCCC] |
| III-18 | 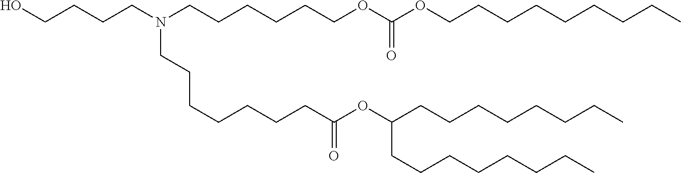 [SMILES: CCCCCCCCCOC(=O)OCCCCCCN(CCCCO)CCCCCCCC(=O)OC(CCCCCCCC)CCCCCCCC] |
| III-19 | 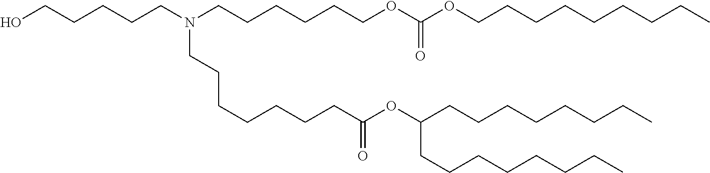 [SMILES: CCCCCCCCCOC(=O)OCCCCCCN(CCCCCO)CCCCCCCC(=O)OC(CCCCCCCC)CCCCCCCC] |
| III-20 | 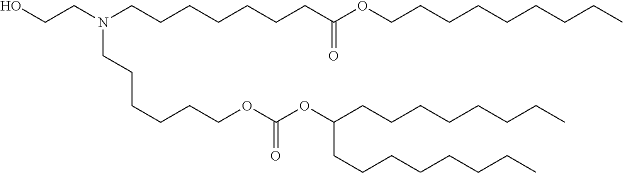 [SMILES: CCCCCCCCCOC(=O)CCCCCCCN(CCO)CCCCCCOC(=O)OC(CCCCCCCC)CCCCCCCC] |
| III-21 | 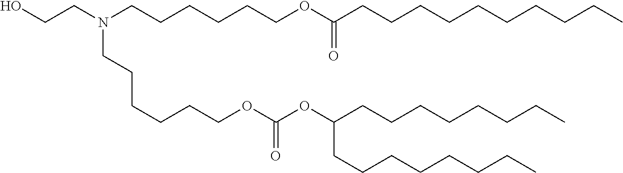 [SMILES: CCCCCCCCCCC(=O)OCCCCCCN(CCO)CCCCCCOC(=O)OC(CCCCCCCC)CCCCCCCC] |
| III-22 | 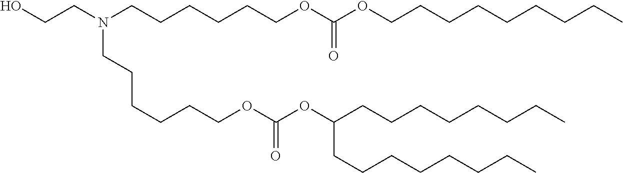 [SMILES: CCCCCCCCCOC(=O)OCCCCCCN(CCO)CCCCCCOC(=O)OC(CCCCCCCC)CCCCCCCC] |
| III-23 | 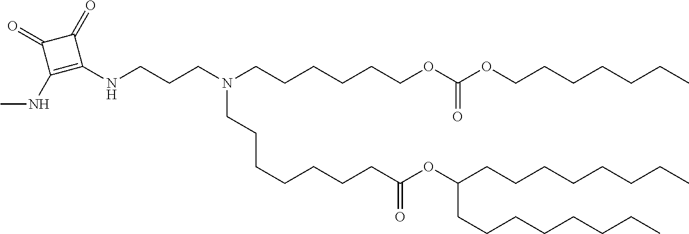 [SMILES: CCCCCCCCC(CCCCCCCC)OC(=O)CCCCCCCN(CCCCCCOC(=O)OCCCCCCC)CCCNc1c(NC)c(=O)c1=O] |
| III-24 | 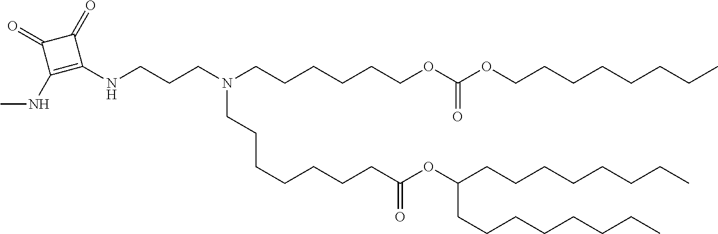 [SMILES: CCCCCCCCOC(=O)OCCCCCCN(CCCCCCCC(=O)OC(CCCCCCCC)CCCCCCCC)CCCNc1c(NC)c(=O)c1=O] |
| III-25 | 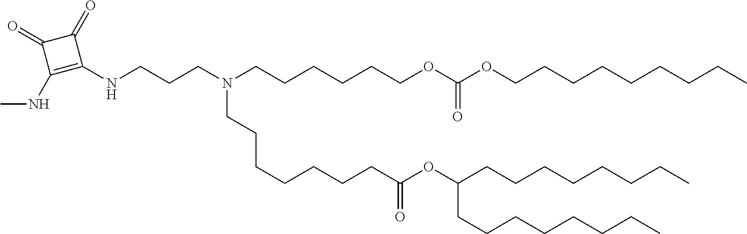 [SMILES: CCCCCCCCCOC(=O)OCCCCCCN(CCCCCCCC(=O)OC(CCCCCCCC)CCCCCCCC)CCCNc1c(NC)c(=O)c1=O] |
| III-26 | 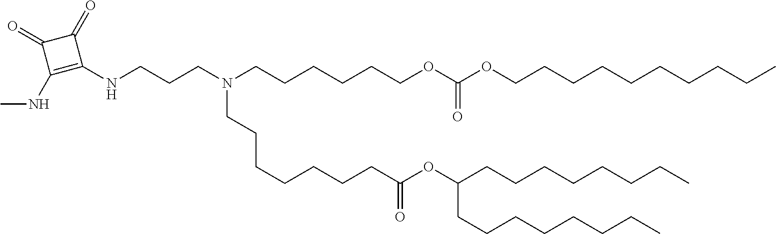 [SMILES: CCCCCCCCCCOC(=O)OCCCCCCN(CCCCCCCC(=O)OC(CCCCCCCC)CCCCCCCC)CCCNc1c(NC)c(=O)c1=O] |
| III-27 | 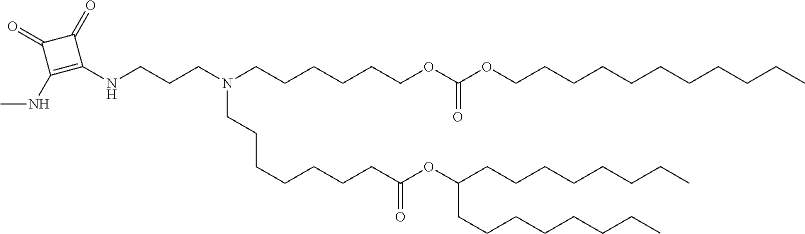 [SMILES: CCCCCCCCCCCOC(=O)OCCCCCCN(CCCCCCCC(=O)OC(CCCCCCCC)CCCCCCCC)CCCNc1c(NC)c(=O)c1=O] |
| III-29 | 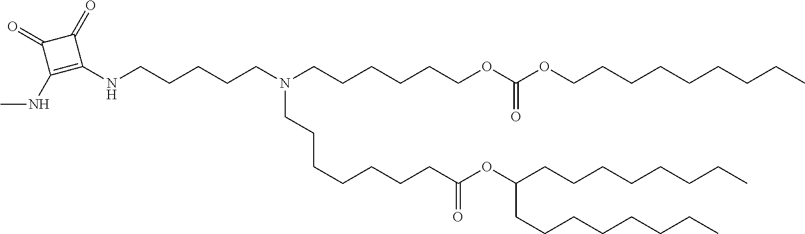 [SMILES: CCCCCCCCCOC(=O)OCCCCCCN(CCCCCCCC(=O)OC(CCCCCCCC)CCCCCCCC)CCCCCNc1c(NC)c(=O)c1=O] |
| III-30 | 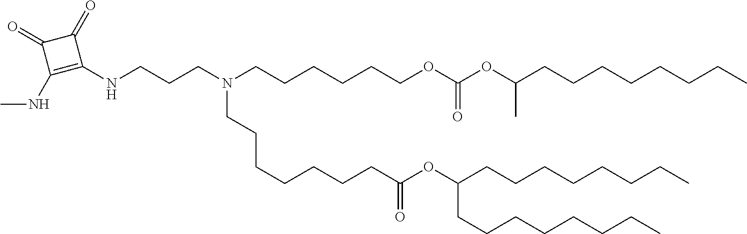 [SMILES: CCCCCCCCC(C)OC(=O)OCCCCCCN(CCCCCCCC(=O)OC(CCCCCCCC)CCCCCCCC)CCCNc1c(NC)c(=O)c1=O] |
| III-31 | 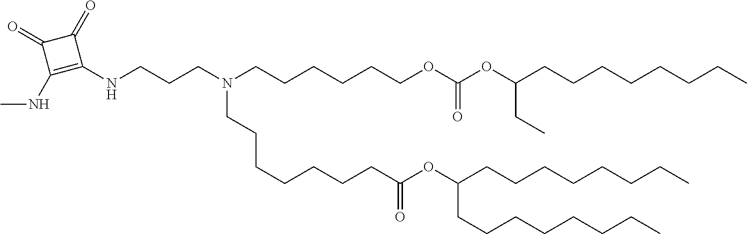 [SMILES: CCCCCCCCC(CCCCCCCC)OC(=O)CCCCCCCN(CCCCCCOC(=O)OC(CC)CCCCCCCC)CCCNc1c(NC)c(=O)c1=O] |
| III-32 | 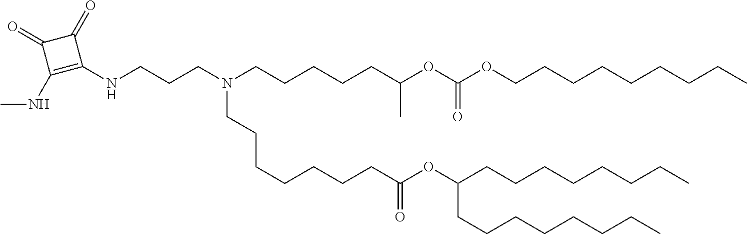 [SMILES: CCCCCCCCCOC(=O)OC(C)CCCCCN(CCCCCCCC(=O)OC(CCCCCCCC)CCCCCCCC)CCCNc1c(NC)c(=O)c1=O] |
| III-33 | 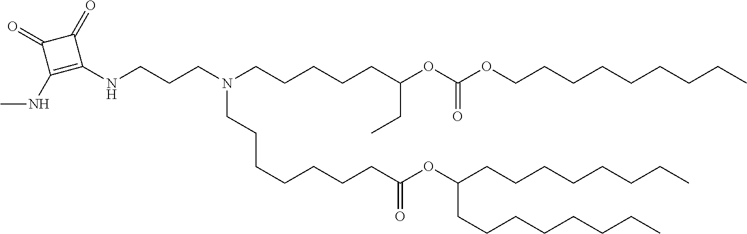 [SMILES: CCCCCCCCCOC(=O)OC(CC)CCCCCN(CCCCCCCC(=O)OC(CCCCCCCC)CCCCCCCC)CCCNc1c(NC)c(=O)c1=O] |
| III-34 | 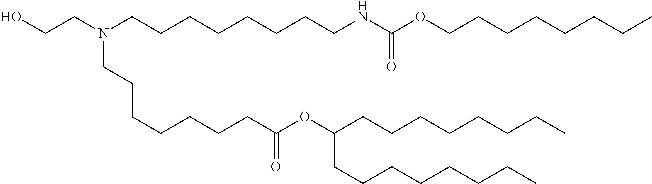 [SMILES: CCCCCCCCOC(=O)NCCCCCCCCN(CCO)CCCCCCCC(=O)OC(CCCCCCCC)CCCCCCCC] |
| III-35 | 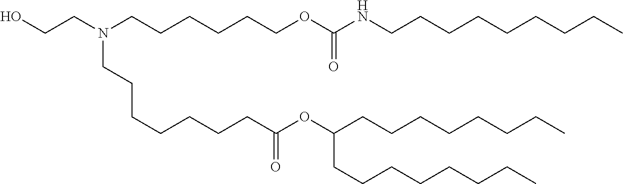 [SMILES: CCCCCCCCCNC(=O)OCCCCCCN(CCO)CCCCCCCC(=O)OC(CCCCCCCC)CCCCCCCC] |
| III-36 | 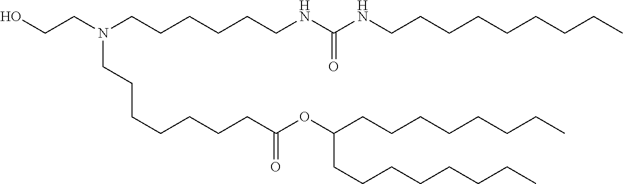 [SMILES: CCCCCCCCCNC(=O)NCCCCCCN(CCO)CCCCCCCC(=O)OC(CCCCCCCC)CCCCCCCC] |
| III-37 | 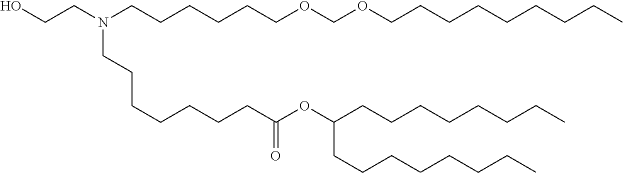 [SMILES: CCCCCCCCCOCOCCCCCCN(CCO)CCCCCCCC(=O)OC(CCCCCCCC)CCCCCCCC] |
| III-38 | 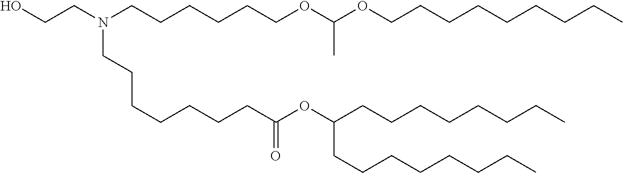 [SMILES: CCCCCCCCCOC(C)OCCCCCCN(CCO)CCCCCCCC(=O)OC(CCCCCCCC)CCCCCCCC] |
| III-39 | 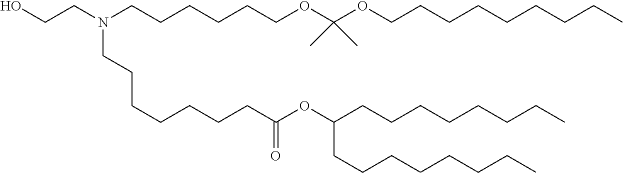 [SMILES: CCCCCCCCCOC(C)(C)OCCCCCCN(CCO)CCCCCCCC(=O)OC(CCCCCCCC)CCCCCCCC] |
| III-40 | 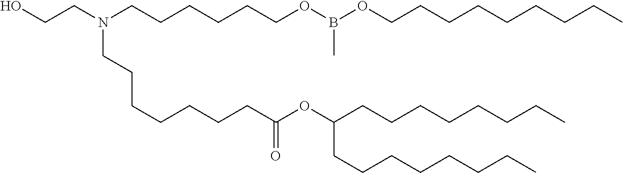 [SMILES: CCCCCCCCCOB(C)OCCCCCCN(CCO)CCCCCCCC(=O)OC(CCCCCCCC)CCCCCCCC] |
| III-41 | 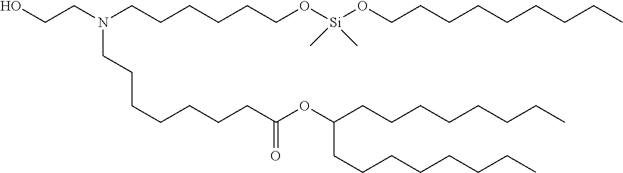 [SMILES: CCCCCCCCCO[Si](C)(C)OCCCCCCN(CCO)CCCCCCCC(=O)OC(CCCCCCCC)CCCCCCCC] |
| III-42 | 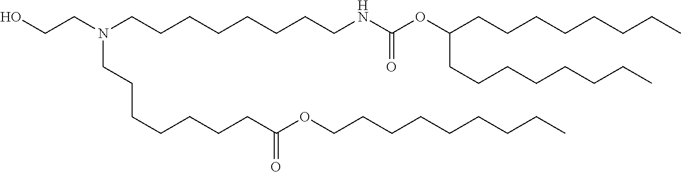 [SMILES: CCCCCCCCCOC(=O)CCCCCCCN(CCO)CCCCCCCCNC(=O)OC(CCCCCCCC)CCCCCCCC] |
| III-43 | 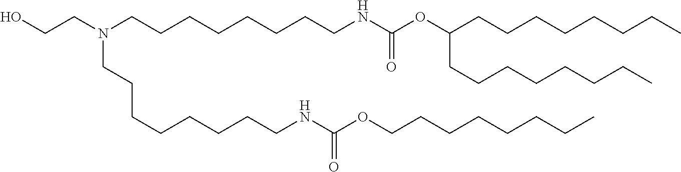 [SMILES: CCCCCCCCOC(=O)NCCCCCCCCN(CCO)CCCCCCCCNC(=O)OC(CCCCCCCC)CCCCCCCC] |
| III-44 | 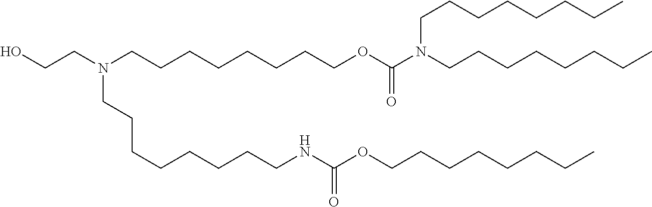 [SMILES: CCCCCCCCOC(=O)NCCCCCCCCN(CCO)CCCCCCCCOC(=O)N(CCCCCCCC)CCCCCCCC] |
| III-45 | 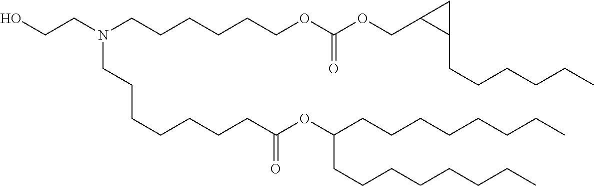 [SMILES: CCCCCCCCC(CCCCCCCC)OC(=O)CCCCCCCN(CCO)CCCCCCOC(=O)OCC1CC1CCCCCC] |
| III-46 | 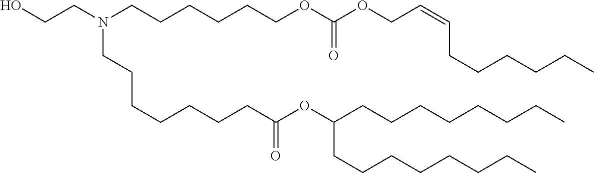 [SMILES: CCCCCC/C=C\COC(=O)OCCCCCCN(CCO)CCCCCCCC(=O)OC(CCCCCCCC)CCCCCCCC] |
| III-47 | 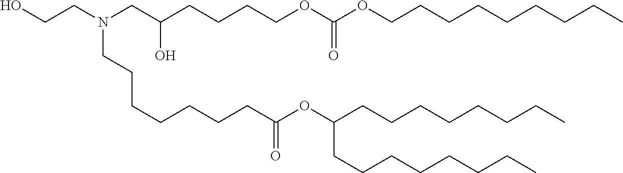 [SMILES: CCCCCCCCCOC(=O)OCCCCC(O)CN(CCO)CCCCCCCC(=O)OC(CCCCCCCC)CCCCCCCC] |
| III-48 | 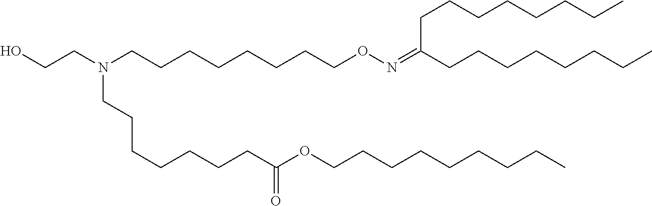 [SMILES: CCCCCCCCCOC(=O)CCCCCCCN(CCO)CCCCCCCCON=C(CCCCCCCC)CCCCCCCC] |
| III-49 | 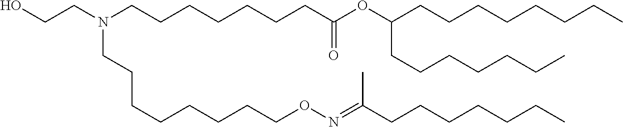 [SMILES: CCCCCCCCC(CCCCCCC)OC(=O)CCCCCCCN(CCO)CCCCCCCCO/N=C(\C)CCCCCCC] |
| III-50 | 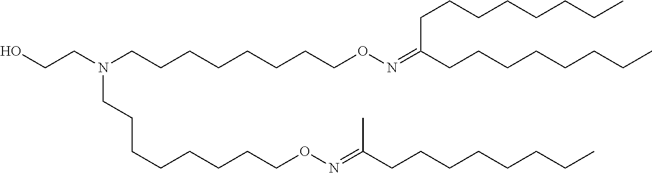 [SMILES: CCCCCCCCC(CCCCCCCC)=NOCCCCCCCCN(CCO)CCCCCCCCO/N=C(\C)CCCCCCCC] |
| III-51 | 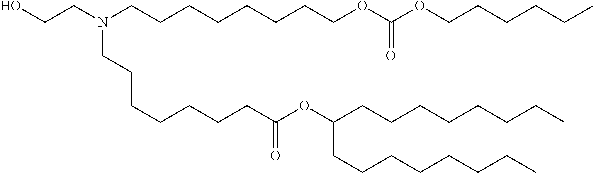 [SMILES: CCCCCCCCC(CCCCCCCC)OC(=O)CCCCCCCN(CCO)CCCCCCCCOC(=O)OCCCCCC] |
| III-52 | 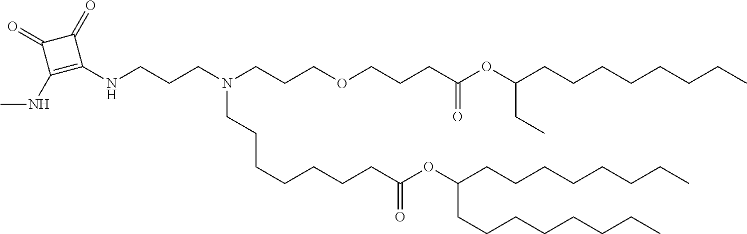 [SMILES: CCCCCCCCC(CC)OC(=O)CCCOCCCN(CCCCCCCC(=O)OC(CCCCCCCC)CCCCCCCC)CCCNc1c(NC)c(=O)c1=O] |
| III-53 | 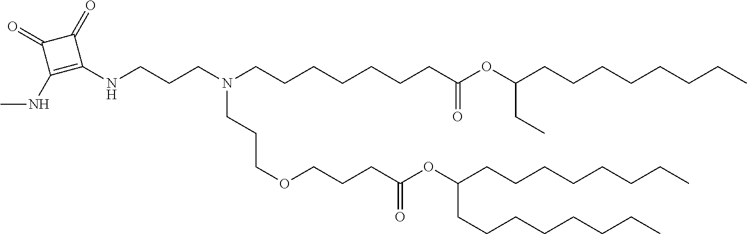 [SMILES: CCCCCCCCC(CC)OC(=O)CCCCCCCN(CCCNc1c(NC)c(=O)c1=O)CCCOCCCC(=O)OC(CCCCCCCC)CCCCCCCC] |
| III-54 | 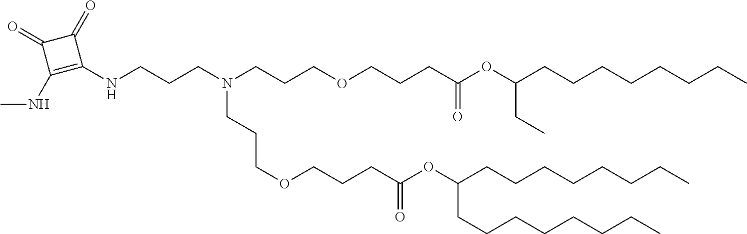 [SMILES: CCCCCCCCC(CC)OC(=O)CCCOCCCN(CCCNc1c(NC)c(=O)c1=O)CCCOCCCC(=O)OC(CCCCCCCC)CCCCCCCC] |
| III-55 | 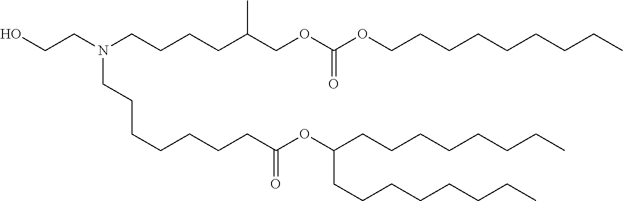 [SMILES: CCCCCCCCCOC(=O)OCC(C)CCCCN(CCO)CCCCCCCC(=O)OC(CCCCCCCC)CCCCCCCC] |
| III-56 | 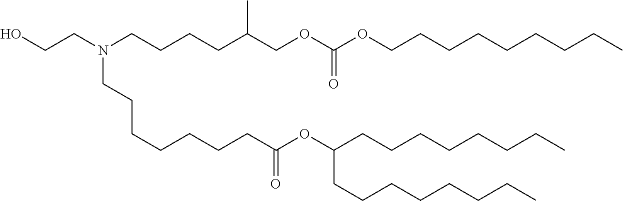 [SMILES: CCCCCCCCCOC(=O)OCC(C)CCCCN(CCO)CCCCCCCC(=O)OC(CCCCCCCC)CCCCCCCC] |
| III-57 | 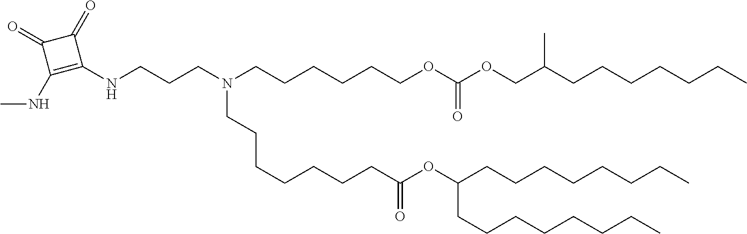 [SMILES: CCCCCCCCC(CCCCCCCC)OC(=O)CCCCCCCN(CCCCCCOC(=O)OCC(C)CCCCCCC)CCCNc1c(NC)c(=O)c1=O] |
| III-58 | 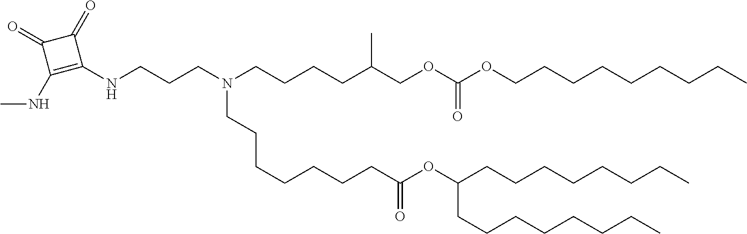 [SMILES: CCCCCCCCCOC(=O)OCC(C)CCCCN(CCCCCCCC(=O)OC(CCCCCCCC)CCCCCCCC)CCCNc1c(NC)c(=O)c1=O] |
| III-59 | 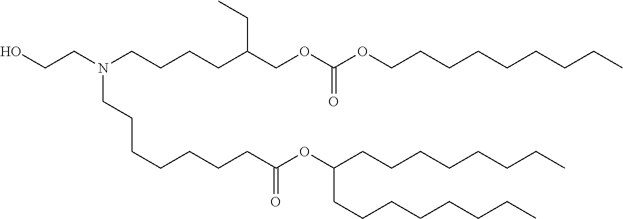 [SMILES: CCCCCCCCCOC(=O)OCC(CC)CCCCN(CCO)CCCCCCCC(=O)OC(CCCCCCCC)CCCCCCCC] |
| III-60 | 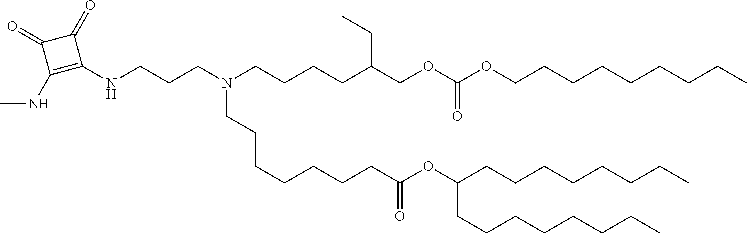 [SMILES: CCCCCCCCCOC(=O)OCC(CC)CCCCN(CCCCCCCC(=O)OC(CCCCCCCC)CCCCCCCC)CCCNc1c(NC)c(=O)c1=O] |
| III-61 | 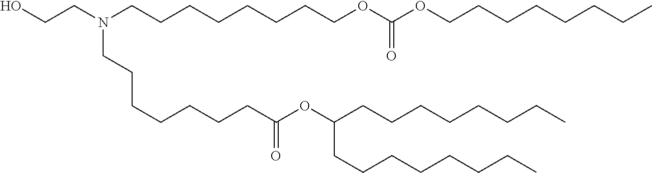 [SMILES: CCCCCCCCOC(=O)OCCCCCCCCN(CCO)CCCCCCCC(=O)OC(CCCCCCCC)CCCCCCCC] |
| III-62 | 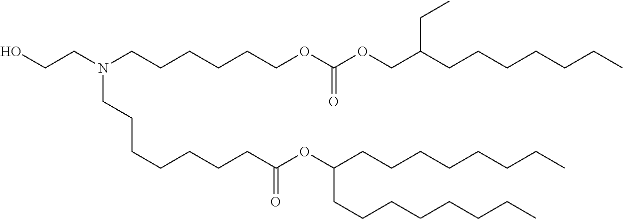 [SMILES: CCCCCCCCC(CCCCCCCC)OC(=O)CCCCCCCN(CCO)CCCCCCOC(=O)OCC(CC)CCCCCCC] |
| III-63 | 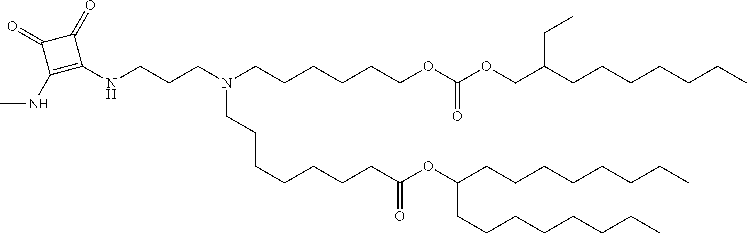 [SMILES: CCCCCCCCC(CCCCCCCC)OC(=O)CCCCCCCN(CCCCCCOC(=O)OCC(CC)CCCCCCC)CCCNc1c(NC)c(=O)c1=O] |
| III-64 | 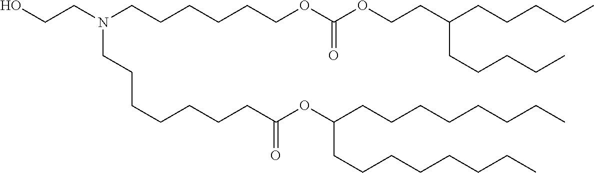 [SMILES: CCCCCCCCC(CCCCCCCC)OC(=O)CCCCCCCN(CCO)CCCCCCOC(=O)OCCC(CCCCC)CCCCC] |
| III-65 | 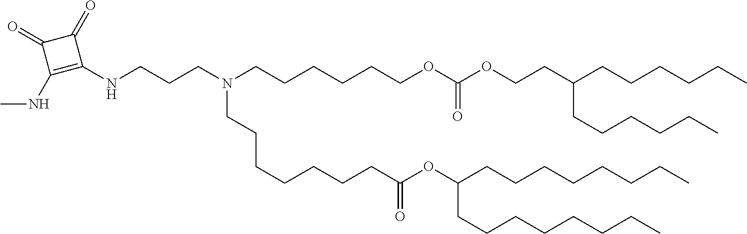 [SMILES: CCCCCCCCC(CCCCCCCC)OC(=O)CCCCCCCN(CCCCCCOC(=O)OCCC(CCCCCC)CCCCCC)CCCNc1c(NC)c(=O)c1=O] |
| III-66 | 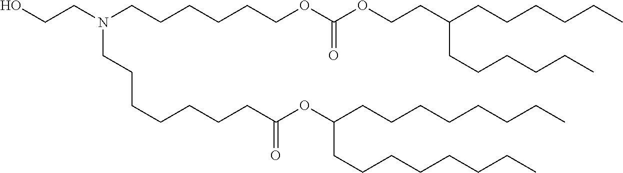 [SMILES: CCCCCCCCC(CCCCCCCC)OC(=O)CCCCCCCN(CCO)CCCCCCOC(=O)OCCC(CCCCCC)CCCCCC] |
| III-67 | 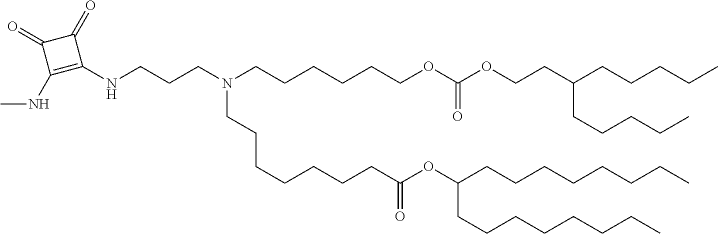 [SMILES: CCCCCCCCC(CCCCCCCC)OC(=O)CCCCCCCN(CCCCCCOC(=O)OCCC(CCCCC)CCCCC)CCCNc1c(NC)c(=O)c1=O] |

In some aspects, the ionizable lipid is a compound of Formula (IL-IIB):

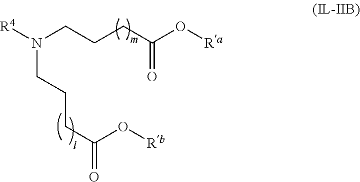

or its N-oxide, or a salt or isomer thereof, wherein R″ is:

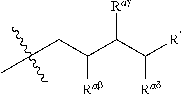

### and R′b is:

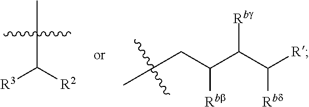

wherein

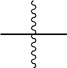

denotes a point of attachment;
R^{aβ}, R^{aγ}, and R^{aδ} are each independently selected from the group consisting of H, C₁₋₁₂ alkyl, and C₂₋₁₂ alkenyl;
R^{bβ}, R^{bγ}, and R^{bδ} are each independently selected from the group consisting of H, C₁₋₁₂ alkyl, and C₂₋₁₂ alkenyl, wherein at least one of R^{bβ}, R^{bγ}, and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from —(CH₂)ₙNRTQ, —(CH₂),NRS(O)₂TQ, —(CH₂)ₙNRC(O)H and —(CH₂)ₙNRC(O)TQ wherein n is selected from 1, 2, 3, 4, and 5;
T is a bond or a C₁₋₃ alkyl linker, C₂₋₃ alkenyl linker, or C₂₋₃ alkynyl linker;
Q is selected from 3-14 membered heterocycle containing 1-5 heteroatoms selected from N, O, and S, C₃₋₁₀ carbocycle, C₁₋₆ alkyl, and C₂₋₆ alkenyl, wherein the alkyl, alkenyl, heterocycle, and carbocycle are each optionally substituted with one or more R^{Q};
each R^{Q} independently is selected from the group consisting of oxo, hydroxyl, cyano, amino, C₁₋₆ alkylamino, di-C₁₋₆ alkylamino, C₁₋₆ alkyl, C₁₋₆ alkoxy, C₂₋₆ alkenyl, C₁₋₆ alkanolyl, —C(O)C₁₋₆ alkyl, and —NRC(O)C₁₋₆ alkyl;
each R is independently selected from H, C₁₋₆ alkyl, and C₂₋₆ alkenyl;
each R′ is independently selected from C₁₋₁₂ alkyl and C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments, the ionizable lipid is a compound selected from Table IL-4.

TABLE IL-4
Ionizable lipids

| Cpd. | |
|---|---|
| # | Structure |
| IV-1 | 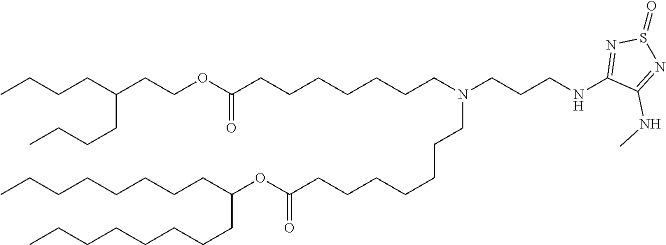 [SMILES: CCCCCCCCC(CCCCCCCC)OC(=O)CCCCCCCN(CCCCCCCC(=O)OCCC(CCCC)CCCC)CCCNC1=NS(=O)N=C1NC] |
| IV-2 | 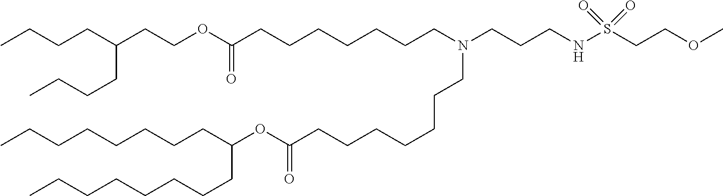 [SMILES: CCCCCCCCC(CCCCCCCC)OC(=O)CCCCCCCN(CCCCCCCC(=O)OCCC(CCCC)CCCC)CCCNS(=O)(=O)CCOC] |
| IV-15 | 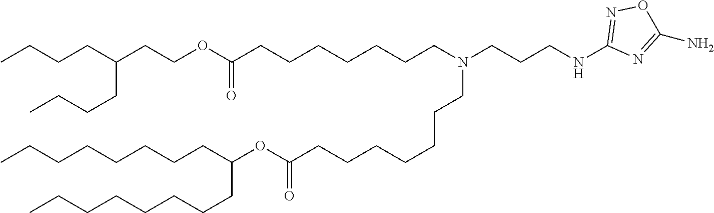 [SMILES: CCCCCCCCC(CCCCCCCC)OC(=O)CCCCCCCN(CCCCCCCC(=O)OCCC(CCCC)CCCC)CCCNc1noc(N)n1] |
| IV-16 | 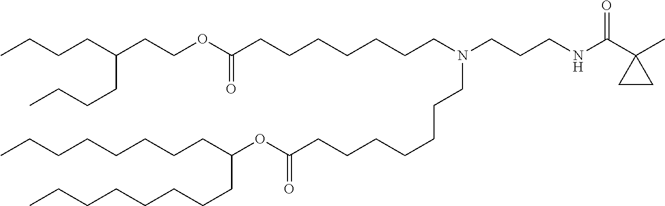 [SMILES: CCCCCCCCC(CCCCCCCC)OC(=O)CCCCCCCN(CCCCCCCC(=O)OCCC(CCCC)CCCC)CCCNC(=O)C1(C)CC1] |
| IV-3 | 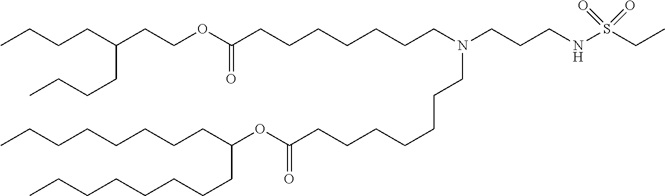 [SMILES: CCCCCCCCC(CCCCCCCC)OC(=O)CCCCCCCN(CCCCCCCC(=O)OCCC(CCCC)CCCC)CCCNS(=O)(=O)CC] |
| IV-4 | 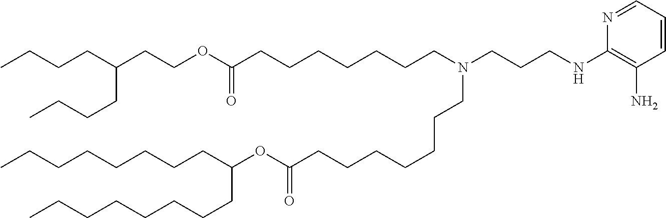 [SMILES: CCCCCCCCC(CCCCCCCC)OC(=O)CCCCCCCN(CCCCCCCC(=O)OCCC(CCCC)CCCC)CCCNc1ncccc1N] |
| IV-5 | 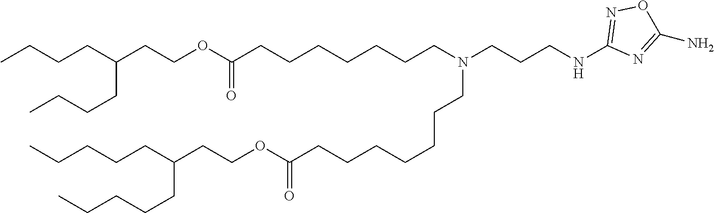 [SMILES: CCCCCC(CCCCC)CCOC(=O)CCCCCCCN(CCCCCCCC(=O)OCCC(CCCC)CCCC)CCCNc1noc(N)n1] |
| IV-6 | 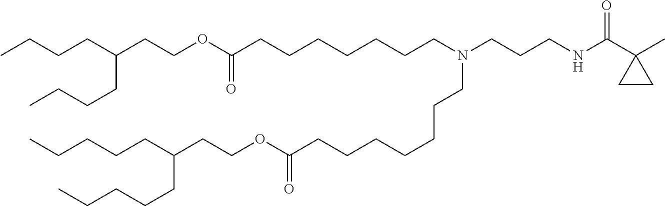 [SMILES: CCCCCC(CCCCC)CCOC(=O)CCCCCCCN(CCCCCCCC(=O)OCCC(CCCC)CCCC)CCCNC(=O)C1(C)CC1] |
| IV-7 | 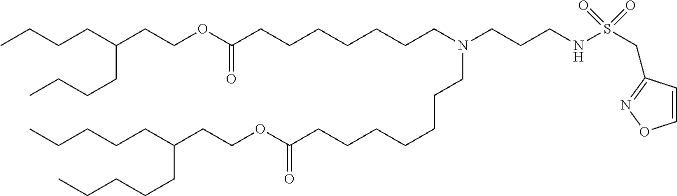 [SMILES: CCCCCC(CCCCC)CCOC(=O)CCCCCCCN(CCCCCCCC(=O)OCCC(CCCC)CCCC)CCCNS(=O)(=O)Cc1ccon1] |
| IV-8 | 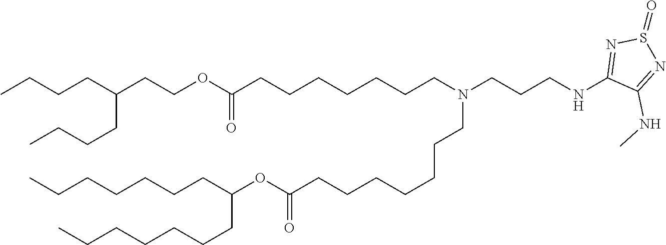 [SMILES: CCCCCCCC(CCCCCCC)OC(=O)CCCCCCCN(CCCCCCCC(=O)OCCC(CCCC)CCCC)CCCNC1=NS(=O)N=C1NC] |
| IV-9 | 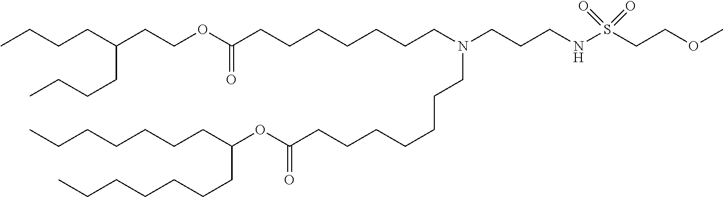 [SMILES: CCCCCCCC(CCCCCCC)OC(=O)CCCCCCCN(CCCCCCCC(=O)OCCC(CCCC)CCCC)CCCNS(=O)(=O)CCOC] |
| IV-10 | 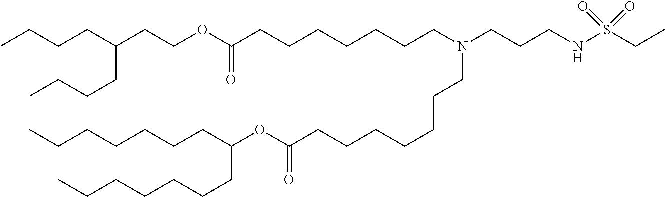 [SMILES: CCCCCCCC(CCCCCCC)OC(=O)CCCCCCCN(CCCCCCCC(=O)OCCC(CCCC)CCCC)CCCNS(=O)(=O)CC] |
| IV-11 | 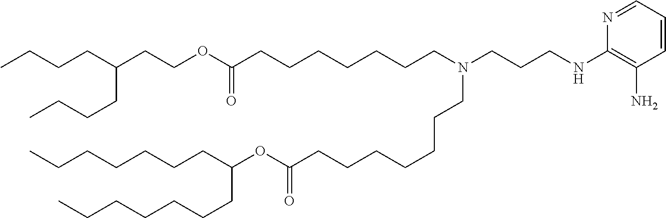 [SMILES: CCCCCCCC(CCCCCCC)OC(=O)CCCCCCCN(CCCCCCCC(=O)OCCC(CCCC)CCCC)CCCNc1ncccc1N] |
| IV-12 | 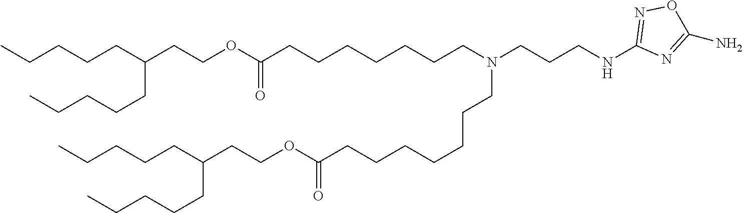 [SMILES: CCCCCC(CCCCC)CCOC(=O)CCCCCCCN(CCCCCCCC(=O)OCCC(CCCCC)CCCCC)CCCNc1noc(N)n1] |
| IV-17 | 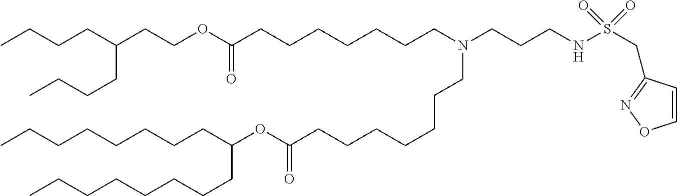 [SMILES: CCCCCCCCC(CCCCCCCC)OC(=O)CCCCCCCN(CCCCCCCC(=O)OCCC(CCCC)CCCC)CCCNS(=O)(=O)Cc1ccon1] |
| IV-18 | 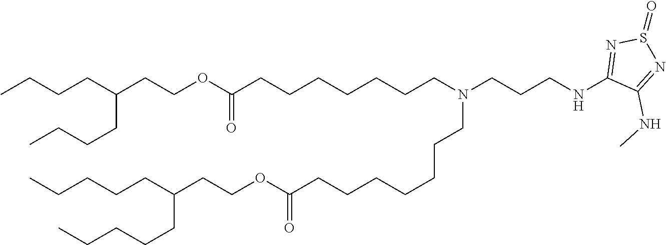 [SMILES: CCCCCC(CCCCC)CCOC(=O)CCCCCCCN(CCCCCCCC(=O)OCCC(CCCC)CCCC)CCCNC1=NS(=O)N=C1NC] |
| IV-19 | 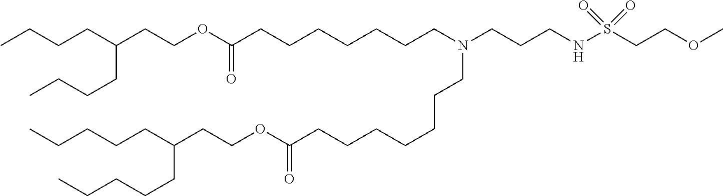 [SMILES: CCCCCC(CCCCC)CCOC(=O)CCCCCCCN(CCCCCCCC(=O)OCCC(CCCC)CCCC)CCCNS(=O)(=O)CCOC] |
| IV-20 | 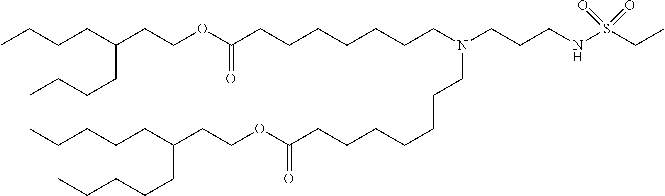 [SMILES: CCCCCC(CCCCC)CCOC(=O)CCCCCCCN(CCCCCCCC(=O)OCCC(CCCC)CCCC)CCCNS(=O)(=O)CC] |
| IV-21 | 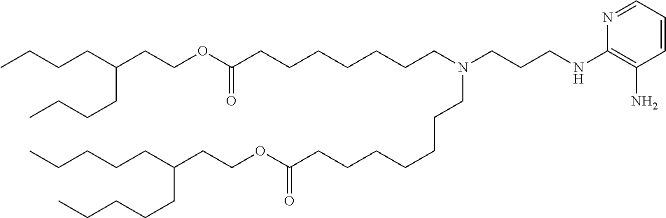 [SMILES: CCCCCC(CCCCC)CCOC(=O)CCCCCCCN(CCCCCCCC(=O)OCCC(CCCC)CCCC)CCCNc1ncccc1N] |
| IV-22 | 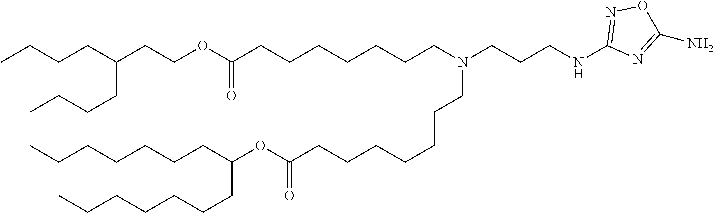 [SMILES: CCCCCCCC(CCCCCCC)OC(=O)CCCCCCCN(CCCCCCCC(=O)OCCC(CCCC)CCCC)CCCNc1noc(N)n1] |
| IV-23 | 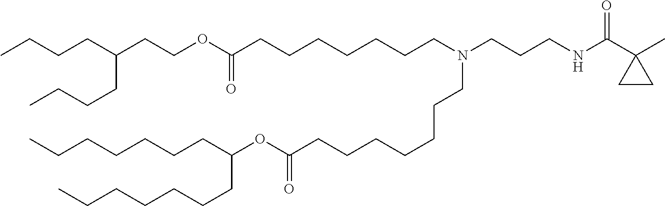 [SMILES: CCCCCCCC(CCCCCCC)OC(=O)CCCCCCCN(CCCCCCCC(=O)OCCC(CCCC)CCCC)CCCNC(=O)C1(C)CC1] |
| IV-24 | 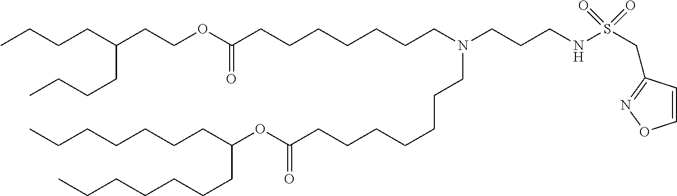 [SMILES: CCCCCCCC(CCCCCCC)OC(=O)CCCCCCCN(CCCCCCCC(=O)OCCC(CCCC)CCCC)CCCNS(=O)(=O)Cc1ccon1] |
| IV-25 | 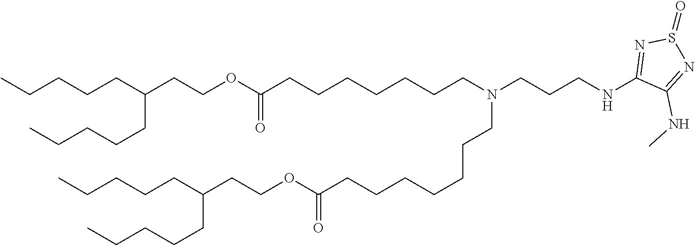 [SMILES: CCCCCC(CCCCC)CCOC(=O)CCCCCCCN(CCCCCCCC(=O)OCCC(CCCCC)CCCCC)CCCNC1=NS(=O)N=C1NC] |
| IV-26 | 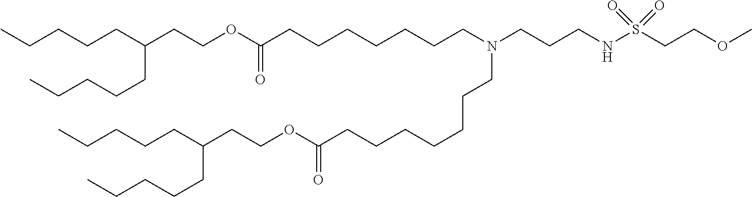 [SMILES: CCCCCC(CCCCC)CCOC(=O)CCCCCCCN(CCCCCCCC(=O)OCCC(CCCCC)CCCCC)CCCNS(=O)(=O)CCOC] |
| IV-13 | 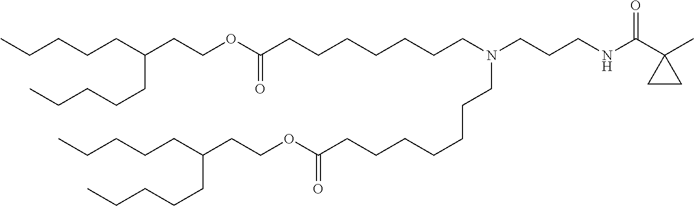 [SMILES: CCCCCC(CCCCC)CCOC(=O)CCCCCCCN(CCCCCCCC(=O)OCCC(CCCCC)CCCCC)CCCNC(=O)C1(C)CC1] |
| IV-14 | 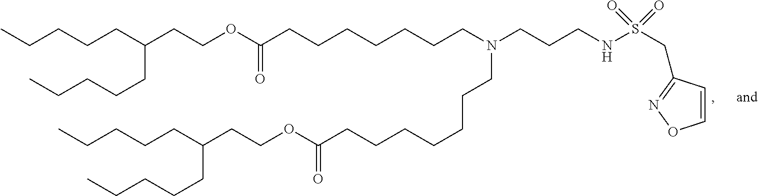 [SMILES: CCCCCC(CCCCC)CCOC(=O)CCCCCCCN(CCCCCCCC(=O)OCCC(CCCCC)CCCCC)CCCNS(=O)(=O)Cc1ccon1] |
| IV-27 | 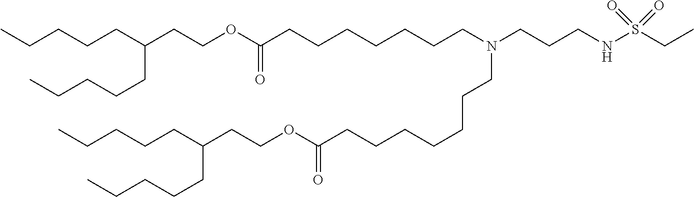 [SMILES: CCCCCC(CCCCC)CCOC(=O)CCCCCCCN(CCCCCCCC(=O)OCCC(CCCCC)CCCCC)CCCNS(=O)(=O)CC] |
| IV-28 | 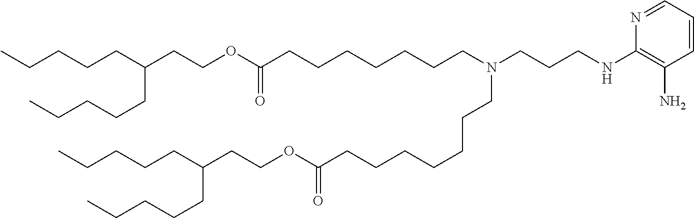 [SMILES: CCCCCC(CCCCC)CCOC(=O)CCCCCCCN(CCCCCCCC(=O)OCCC(CCCCC)CCCCC)CCCNc1ncccc1N] |

In some embodiments, the ionizable lipid is a compound selected from Table IL-5.

TABLE IL-5
Ionizable lipids

| Cpd. | |
|---|---|
| # | Structure |
| IV-29 | 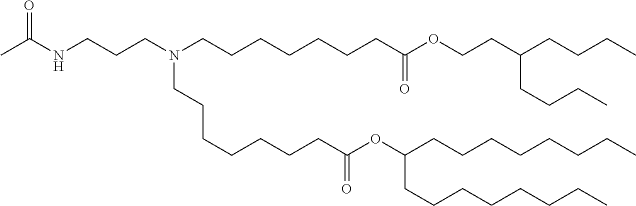 [SMILES: CCCCCCCCC(CCCCCCCC)OC(=O)CCCCCCCN(CCCCCCCC(=O)OCCC(CCCC)CCCC)CCCNC(C)=O] |
| IV-30 | 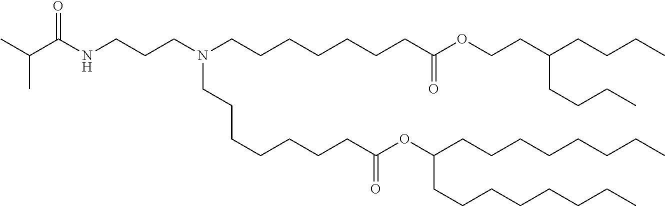 [SMILES: CCCCCCCCC(CCCCCCCC)OC(=O)CCCCCCCN(CCCCCCCC(=O)OCCC(CCCC)CCCC)CCCNC(=O)C(C)C] |
| IV-31 | 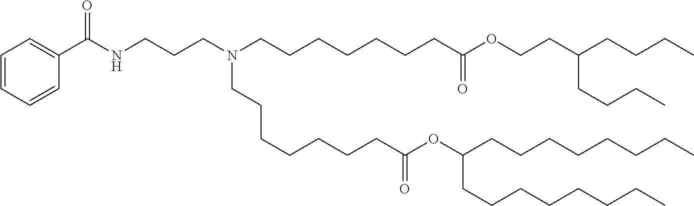 [SMILES: CCCCCCCCC(CCCCCCCC)OC(=O)CCCCCCCN(CCCCCCCC(=O)OCCC(CCCC)CCCC)CCCNC(=O)c1ccccc1] |
| IV-32 | 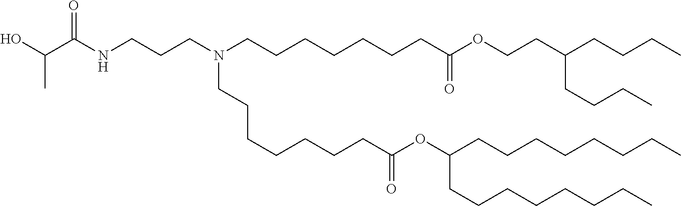 [SMILES: CCCCCCCCC(CCCCCCCC)OC(=O)CCCCCCCN(CCCCCCCC(=O)OCCC(CCCC)CCCC)CCCNC(=O)C(C)O] |
| IV-33 | 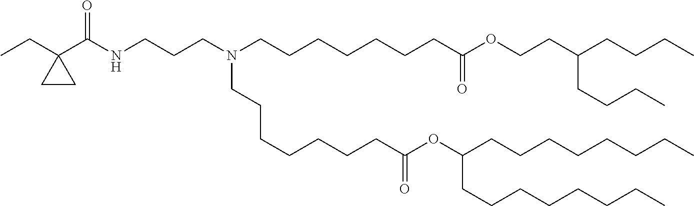 [SMILES: CCCCCCCCC(CCCCCCCC)OC(=O)CCCCCCCN(CCCCCCCC(=O)OCCC(CCCC)CCCC)CCCNC(=O)C1(CC)CC1] |
| IV-34 | 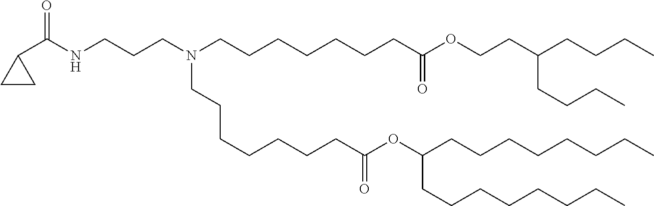 [SMILES: CCCCCCCCC(CCCCCCCC)OC(=O)CCCCCCCN(CCCCCCCC(=O)OCCC(CCCC)CCCC)CCCNC(=O)C1CC1] |
| IV-46 | 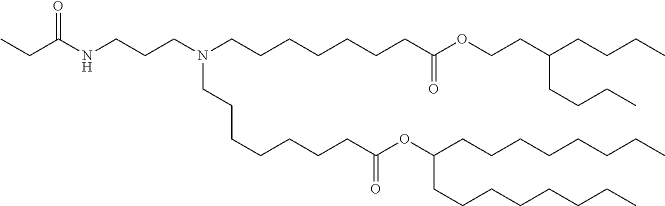 [SMILES: CCCCCCCCC(CCCCCCCC)OC(=O)CCCCCCCN(CCCCCCCC(=O)OCCC(CCCC)CCCC)CCCNC(=O)CC] |
| IV-47 | 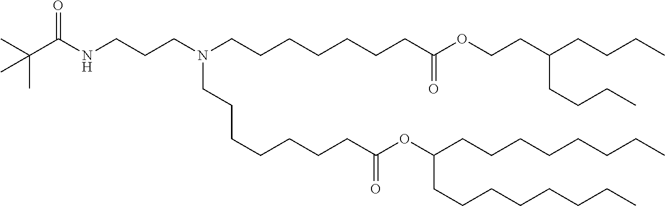 [SMILES: CCCCCCCCC(CCCCCCCC)OC(=O)CCCCCCCN(CCCCCCCC(=O)OCCC(CCCC)CCCC)CCCNC(=O)C(C)(C)C] |
| IV-48 | 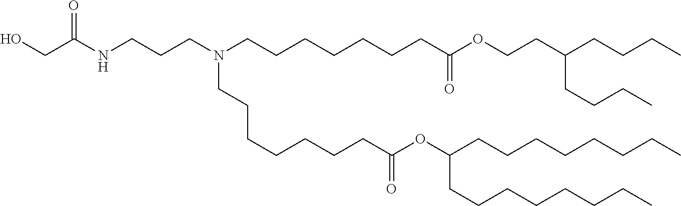 [SMILES: CCCCCCCCC(CCCCCCCC)OC(=O)CCCCCCCN(CCCCCCCC(=O)OCCC(CCCC)CCCC)CCCNC(=O)CO] |
| IV-49 | 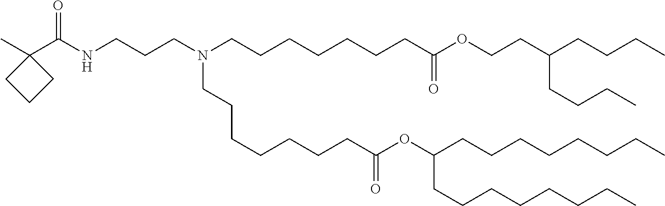 [SMILES: CCCCCCCCC(CCCCCCCC)OC(=O)CCCCCCCN(CCCCCCCC(=O)OCCC(CCCC)CCCC)CCCNC(=O)C1(C)CCC1] |
| IV-50 | 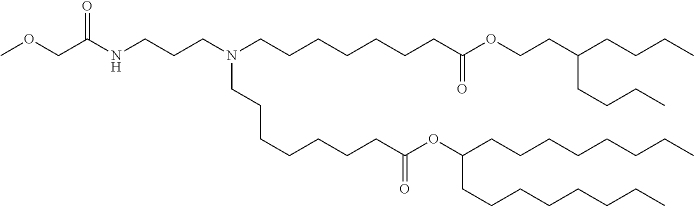 [SMILES: CCCCCCCCC(CCCCCCCC)OC(=O)CCCCCCCN(CCCCCCCC(=O)OCCC(CCCC)CCCC)CCCNC(=O)COC] |
| IV-51 | 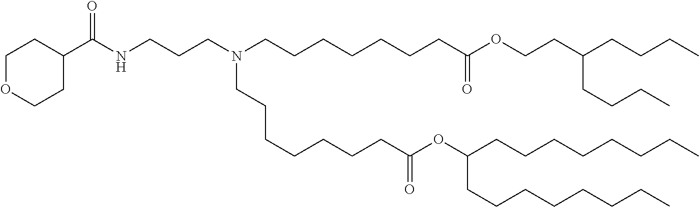 [SMILES: CCCCCCCCC(CCCCCCCC)OC(=O)CCCCCCCN(CCCCCCCC(=O)OCCC(CCCC)CCCC)CCCNC(=O)C1CCOCC1] |
| IV-35 | 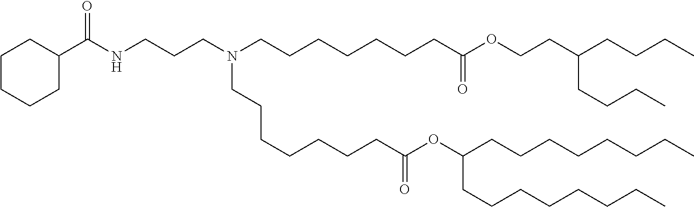 [SMILES: CCCCCCCCC(CCCCCCCC)OC(=O)CCCCCCCN(CCCCCCCC(=O)OCCC(CCCC)CCCC)CCCNC(=O)C1CCCCC1] |
| IV-36 | 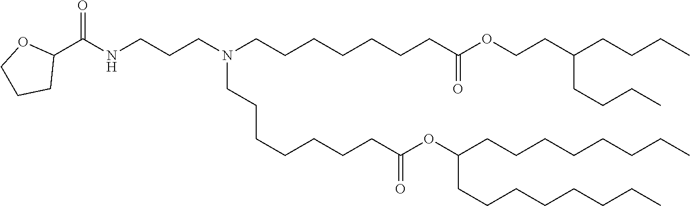 [SMILES: CCCCCCCCC(CCCCCCCC)OC(=O)CCCCCCCN(CCCCCCCC(=O)OCCC(CCCC)CCCC)CCCNC(=O)C1CCCO1] |
| IV-37 | 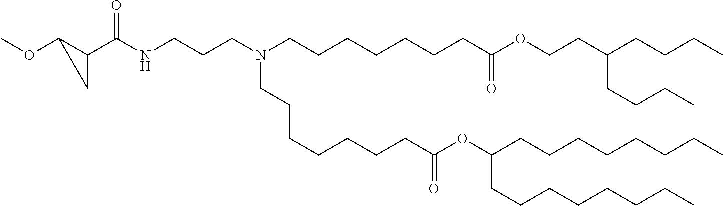 [SMILES: CCCCCCCCC(CCCCCCCC)OC(=O)CCCCCCCN(CCCCCCCC(=O)OCCC(CCCC)CCCC)CCCNC(=O)C1CC1OC] |
| IV-38 | 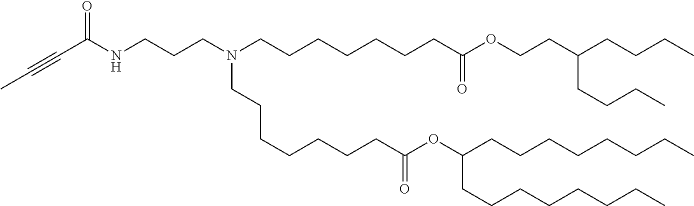 [SMILES: CC#CC(=O)NCCCN(CCCCCCCC(=O)OCCC(CCCC)CCCC)CCCCCCCC(=O)OC(CCCCCCCC)CCCCCCCC] |
| IV-39 | 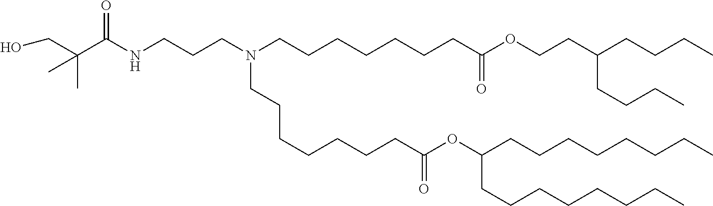 [SMILES: CCCCCCCCC(CCCCCCCC)OC(=O)CCCCCCCN(CCCCCCCC(=O)OCCC(CCCC)CCCC)CCCNC(=O)C(C)(C)CO] |
| IV-40 | 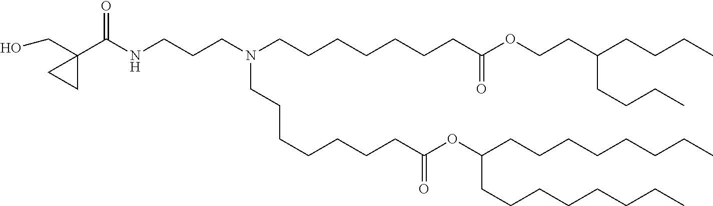 [SMILES: CCCCCCCCC(CCCCCCCC)OC(=O)CCCCCCCN(CCCCCCCC(=O)OCCC(CCCC)CCCC)CCCNC(=O)C1(CO)CC1] |
| IV-41 | 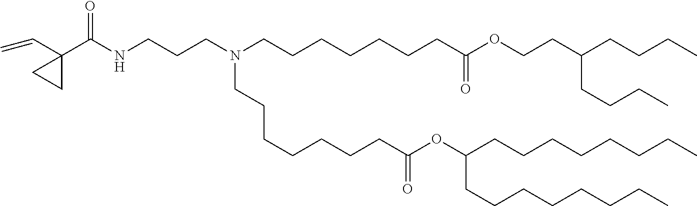 [SMILES: C=CC1(C(=O)NCCCN(CCCCCCCC(=O)OCCC(CCCC)CCCC)CCCCCCCC(=O)OC(CCCCCCCC)CCCCCCCC)CC1] |
| IV-42 | 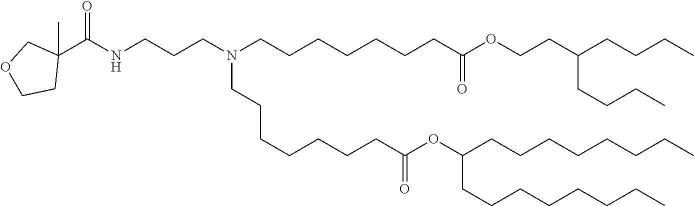 [SMILES: CCCCCCCCC(CCCCCCCC)OC(=O)CCCCCCCN(CCCCCCCC(=O)OCCC(CCCC)CCCC)CCCNC(=O)C1(C)CCOC1] |
| IV-43 | 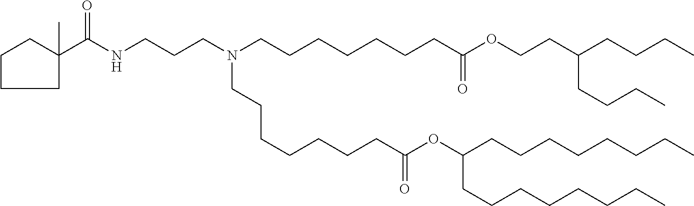 [SMILES: CCCCCCCCC(CCCCCCCC)OC(=O)CCCCCCCN(CCCCCCCC(=O)OCCC(CCCC)CCCC)CCCNC(=O)C1(C)CCCC1] |
| IV-44 | 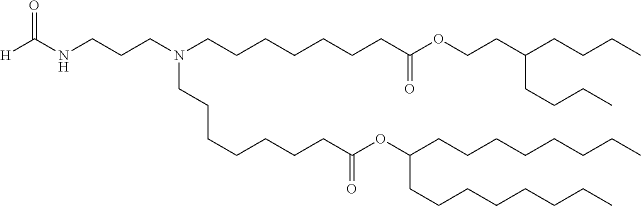 [SMILES: CCCCCCCCC(CCCCCCCC)OC(=O)CCCCCCCN(CCCCCCCC(=O)OCCC(CCCC)CCCC)CCCNC=O] |
| IV-52 | 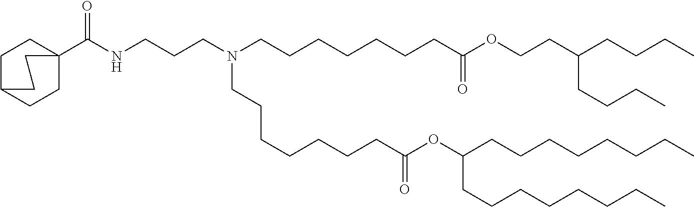 [SMILES: CCCCCCCCC(CCCCCCCC)OC(=O)CCCCCCCN(CCCCCCCC(=O)OCCC(CCCC)CCCC)CCCNC(=O)C12CCC(CC1)CC2] |
| IV-53 | 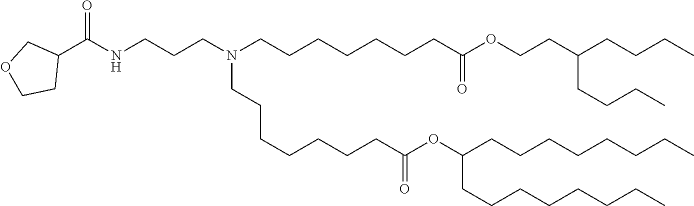 [SMILES: CCCCCCCCC(CCCCCCCC)OC(=O)CCCCCCCN(CCCCCCCC(=O)OCCC(CCCC)CCCC)CCCNC(=O)C1CCOC1] |
| IV-54 | 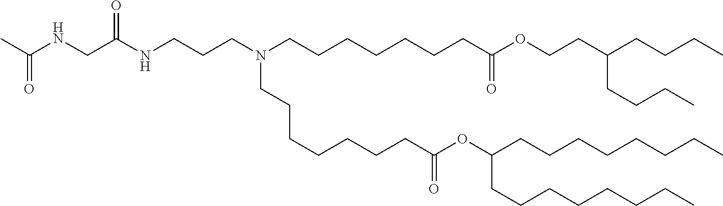 [SMILES: CCCCCCCCC(CCCCCCCC)OC(=O)CCCCCCCN(CCCCCCCC(=O)OCCC(CCCC)CCCC)CCCNC(=O)CNC(C)=O] |
| IV-55 | 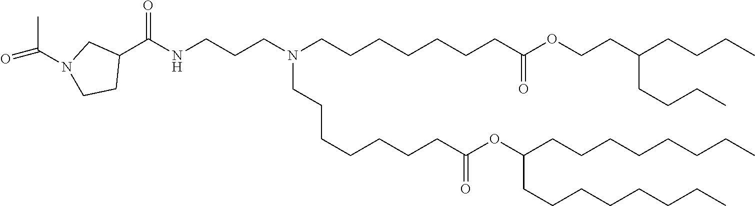 [SMILES: CCCCCCCCC(CCCCCCCC)OC(=O)CCCCCCCN(CCCCCCCC(=O)OCCC(CCCC)CCCC)CCCNC(=O)C1CCN(C(C)=O)C1] |
| IV-56 | 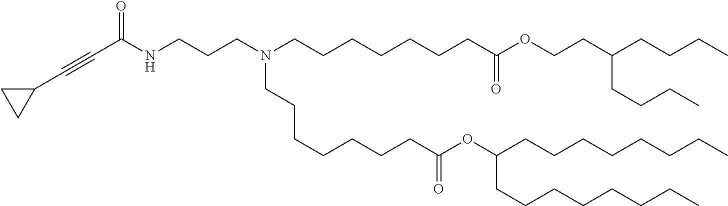 [SMILES: CCCCCCCCC(CCCCCCCC)OC(=O)CCCCCCCN(CCCCCCCC(=O)OCCC(CCCC)CCCC)CCCNC(=O)C#CC1CC1] |
| IV-57 | 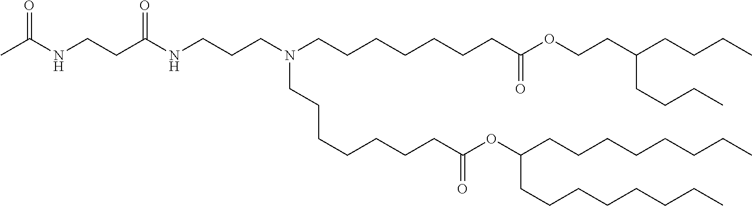 [SMILES: CCCCCCCCC(CCCCCCCC)OC(=O)CCCCCCCN(CCCCCCCC(=O)OCCC(CCCC)CCCC)CCCNC(=O)CCNC(C)=O] |
| IV-58 | 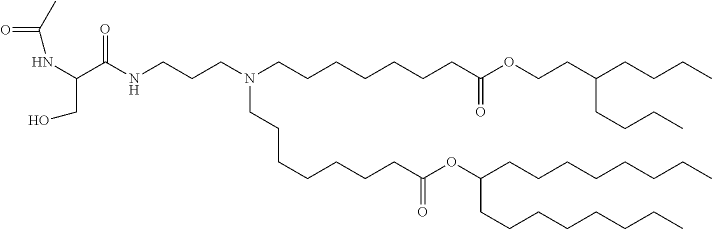 [SMILES: CCCCCCCCC(CCCCCCCC)OC(=O)CCCCCCCN(CCCCCCCC(=O)OCCC(CCCC)CCCC)CCCNC(=O)C(CO)NC(C)=O] |
| IV-59 | 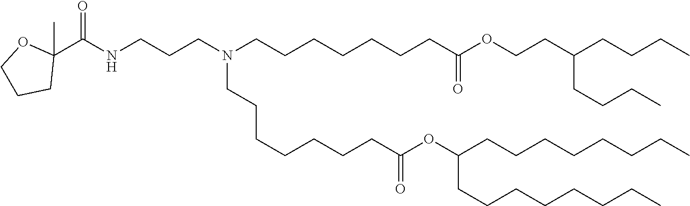 [SMILES: CCCCCCCCC(CCCCCCCC)OC(=O)CCCCCCCN(CCCCCCCC(=O)OCCC(CCCC)CCCC)CCCNC(=O)C1(C)CCCO1] |
| IV-60 | 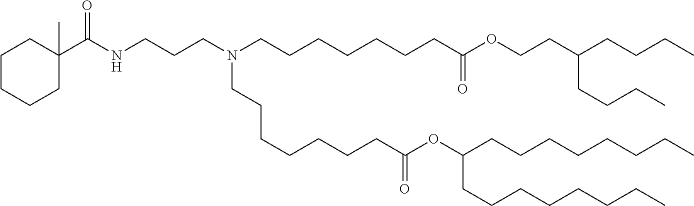 [SMILES: CCCCCCCCC(CCCCCCCC)OC(=O)CCCCCCCN(CCCCCCCC(=O)OCCC(CCCC)CCCC)CCCNC(=O)C1(C)CCCCC1] |
| IV-61 | 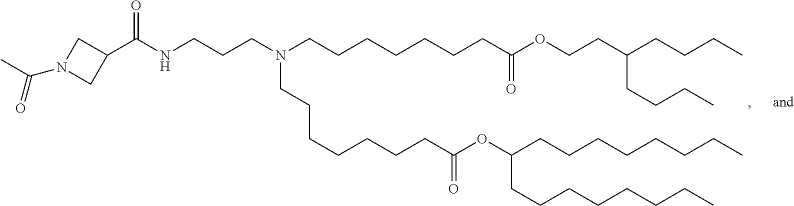 [SMILES: CCCCCCCCC(CCCCCCCC)OC(=O)CCCCCCCN(CCCCCCCC(=O)OCCC(CCCC)CCCC)CCCNC(=O)C1CN(C(C)=O)C1] |
| IV-45 | 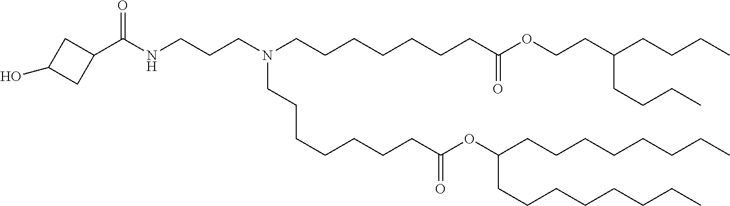 [SMILES: CCCCCCCCC(CCCCCCCC)OC(=O)CCCCCCCN(CCCCCCCC(=O)OCCC(CCCC)CCCC)CCCNC(=O)C1CC(O)C1] |

In some aspects, the ionizable lipid is a compound of Formula (IL-IIC):

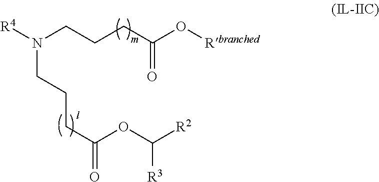

or its N-oxide, or a salt or isomer thereof,
wherein:
R′^{branched} is

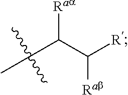

wherein

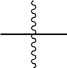

denotes a point of attachment;
wherein R^{aα} and R^{aβ} are each independently selected from the group consisting of H and C₁₋₂ alkyl, wherein at least one of R^{aα} and R^{aβ} is a C₁ or C₂ alkyl;
R′ is selected from the group consisting of C₁₋₁₈ alkyl and C₂₋₁₈ alkenyl;
R² and R³ are each independently selected from the group consisting of C₁₋₁₄ alkyl and C₂₋₁₄ alkenyl;
R⁴ is —(CH₂)ₙQ, wherein n is independently selected from 1, 2, 3, 4, and 5, where Q is selected from NRS(O)₂R^{SX} and

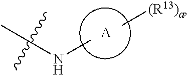

wherein A is a 3-14 membered heterocycle containing one or more heteroatoms selected from N, O and S; and a is 1, 2, 3, or 4; wherein

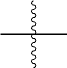

denotes a point of attachment;
R is selected from H and C₁₋₃ alkyl;
R^{SX} is selected from a C₃₋₈ carbocycle, a 3-14 membered heterocycle containing one or more heteroatoms selected from N, O and S, C₁₋₆ alkyl, C₂₋₆ alkenyl, (C₁₋₃ alkoxy)C₁₋₃ alkyl, (CH₂)ₚ₁O(CH₂)ₚ₂R^{SX1}, and (CH₂)ₚ₁R^{SX1}, wherein the carbocycle and heterocycle are optionally substituted with one or more groups selected from oxo, C₁₋₆ alkyl, and (C₁₋₃ alkoxy)C₁₋₃ alkyl;
R^{SX1} is selected from C(O)NR¹⁴R^{14′}, a C₃₋₈ carbocycle, and a 3-14 membered heterocycle containing one or more heteroatoms selected from N, O and S, wherein the carbocycle and heterocycle are each optionally substituted with one or more groups selected from oxo, halo, C₁₋₃ alkyl, (C₁₋₃ alkoxy)C₁₋₃ alkyl, C₁₋₆ alkylamino, di-(C₁₋₆ alkyl) amino, and NH₂;
each R¹³ is selected from the group consisting of OH, oxo, halo, C₁₋₆ alkyl, C₁₋₆ alkoxy, C₂₋₆ alkenyl, C₁₋₆ alkylamino, di-(C₁₋₆ alkyl) amino, NH₂, C(O)NH₂, CN, and NO₂;
R¹⁴ and R^{14′} are each independently selected from the group consisting of H and C₁₋₆ alkyl;
m is selected from 1, 2, 3, 4, 5, 6, 7, 8, and 9;
l is selected from 1, 2, 3, 4, 5, 6, 7, 8, and 9;
p₁ is selected from 1, 2, 3, 4, and 5; and
p₂ is selected from 1, 2, 3, 4, and 5.

In some embodiments, the ionizable lipid is a compound selected from Table TL-6.

TABLE IL-6
Ionizable lipids

| Cpd | |
|---|---|
| # | Structure |
| V-1 | 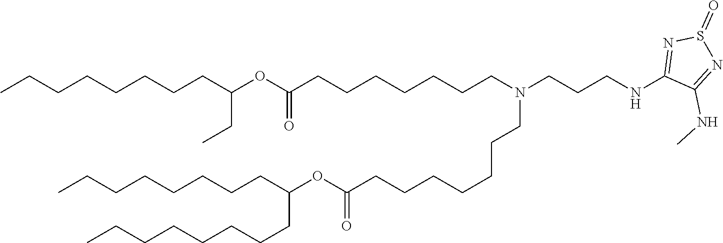 [SMILES: CCCCCCCCC(CC)OC(=O)CCCCCCCN(CCCCCCCC(=O)OC(CCCCCCCC)CCCCCCCC)CCCNC1=NS(=O)N=C1NC] |
| V-2 | 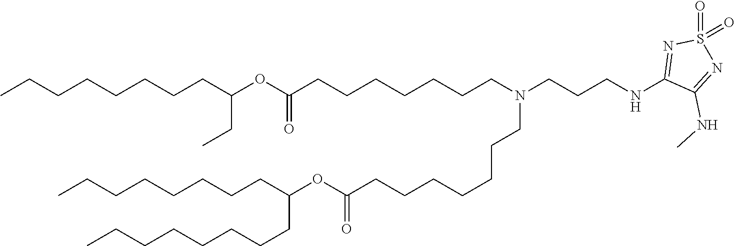 [SMILES: CCCCCCCCC(CC)OC(=O)CCCCCCCN(CCCCCCCC(=O)OC(CCCCCCCC)CCCCCCCC)CCCNC1=NS(=O)(=O)N=C1NC] |
| V-3 | 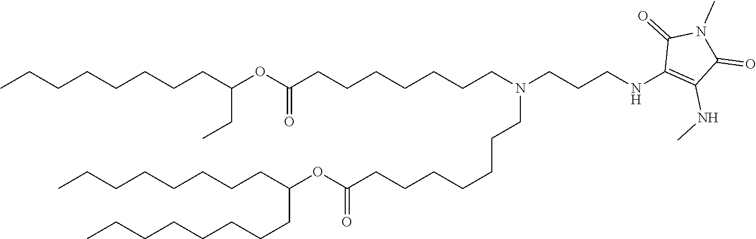 [SMILES: CCCCCCCCC(CC)OC(=O)CCCCCCCN(CCCCCCCC(=O)OC(CCCCCCCC)CCCCCCCC)CCCNC1=C(NC)C(=O)N(C)C1=O] |
| V-4 | 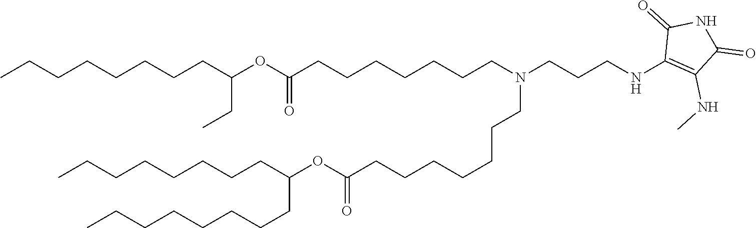 [SMILES: CCCCCCCCC(CC)OC(=O)CCCCCCCN(CCCCCCCC(=O)OC(CCCCCCCC)CCCCCCCC)CCCNC1=C(NC)C(=O)NC1=O] |
| V-5 | 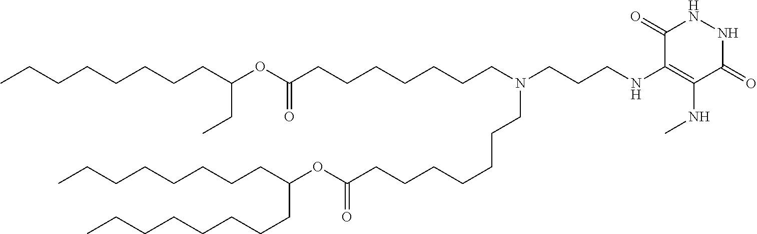 [SMILES: CCCCCCCCC(CC)OC(=O)CCCCCCCN(CCCCCCCC(=O)OC(CCCCCCCC)CCCCCCCC)CCCNc1c(NC)c(=O)[nH][nH]c1=O] |
| V-6 | 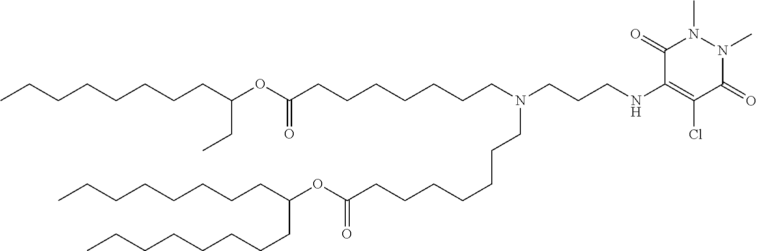 [SMILES: CCCCCCCCC(CC)OC(=O)CCCCCCCN(CCCCCCCC(=O)OC(CCCCCCCC)CCCCCCCC)CCCNc1c(Cl)c(=O)n(C)n(C)c1=O] |
| V-7 | 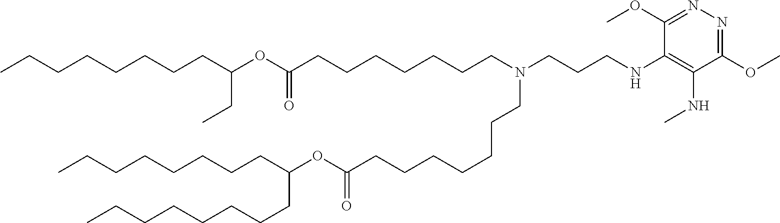 [SMILES: CCCCCCCCC(CC)OC(=O)CCCCCCCN(CCCCCCCC(=O)OC(CCCCCCCC)CCCCCCCC)CCCNc1c(OC)nnc(OC)c1NC] |
| V-8 | 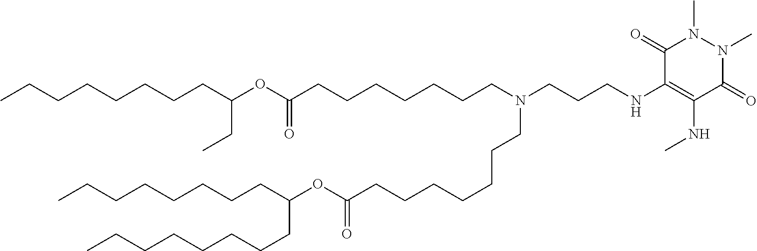 [SMILES: CCCCCCCCC(CC)OC(=O)CCCCCCCN(CCCCCCCC(=O)OC(CCCCCCCC)CCCCCCCC)CCCNc1c(NC)c(=O)n(C)n(C)c1=O] |
| V-9 | 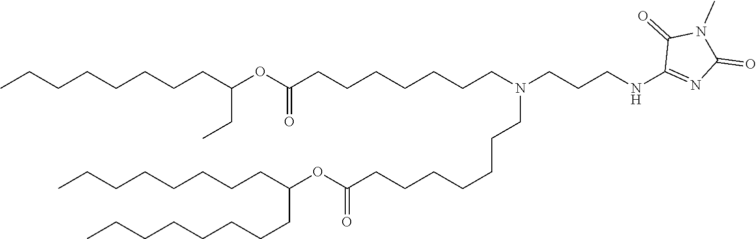 [SMILES: CCCCCCCCC(CC)OC(=O)CCCCCCCN(CCCCCCCC(=O)OC(CCCCCCCC)CCCCCCCC)CCCNC1=NC(=O)N(C)C1=O] |
| V-10 | 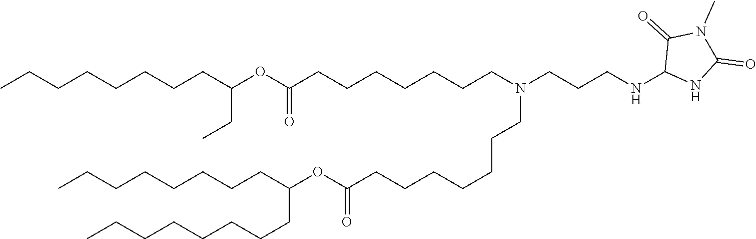 [SMILES: CCCCCCCCC(CC)OC(=O)CCCCCCCN(CCCCCCCC(=O)OC(CCCCCCCC)CCCCCCCC)CCCNC1NC(=O)N(C)C1=O] |
| V-11 | 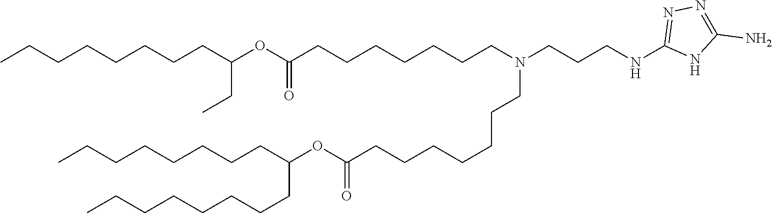 [SMILES: CCCCCCCCC(CC)OC(=O)CCCCCCCN(CCCCCCCC(=O)OC(CCCCCCCC)CCCCCCCC)CCCNc1nnc(N)[nH]1] |
| V-12 | 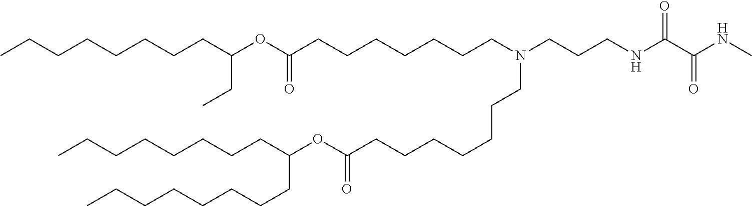 [SMILES: CCCCCCCCC(CC)OC(=O)CCCCCCCN(CCCCCCCC(=O)OC(CCCCCCCC)CCCCCCCC)CCCNC(=O)C(=O)NC] |
| V-13 | 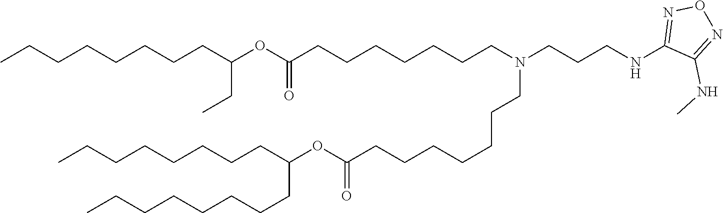 [SMILES: CCCCCCCCC(CC)OC(=O)CCCCCCCN(CCCCCCCC(=O)OC(CCCCCCCC)CCCCCCCC)CCCNc1nonc1NC] |
| V-14 | 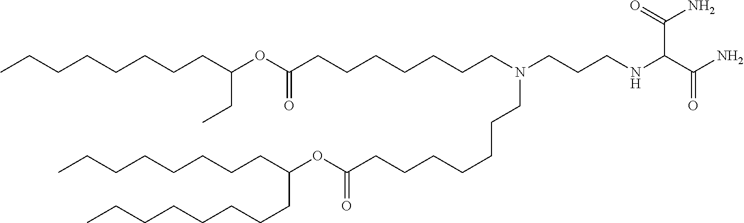 [SMILES: CCCCCCCCC(CC)OC(=O)CCCCCCCN(CCCCCCCC(=O)OC(CCCCCCCC)CCCCCCCC)CCCNC(C(N)=O)C(N)=O] |
| V-15 | 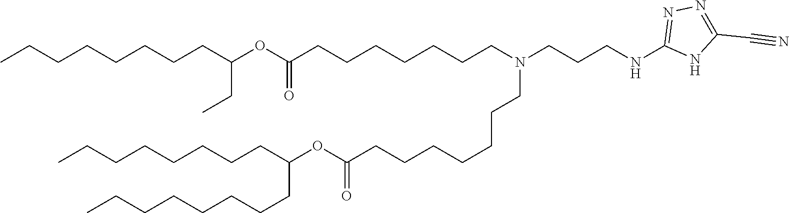 [SMILES: CCCCCCCCC(CC)OC(=O)CCCCCCCN(CCCCCCCC(=O)OC(CCCCCCCC)CCCCCCCC)CCCNc1nnc(C#N)[nH]1] |
| V-16 | 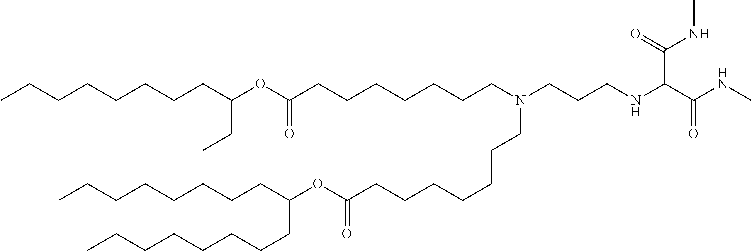 [SMILES: CCCCCCCCC(CC)OC(=O)CCCCCCCN(CCCCCCCC(=O)OC(CCCCCCCC)CCCCCCCC)CCCNC(C(=O)NC)C(=O)NC] |
| V-17 | 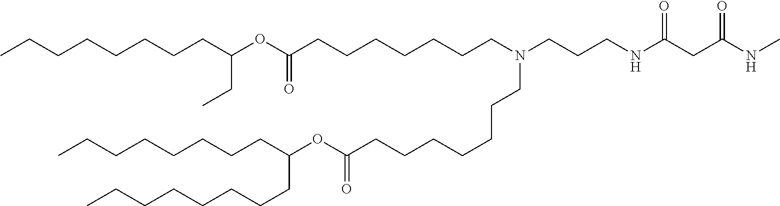 [SMILES: CCCCCCCCC(CC)OC(=O)CCCCCCCN(CCCCCCCC(=O)OC(CCCCCCCC)CCCCCCCC)CCCNC(=O)CC(=O)NC] |
| V-18 | 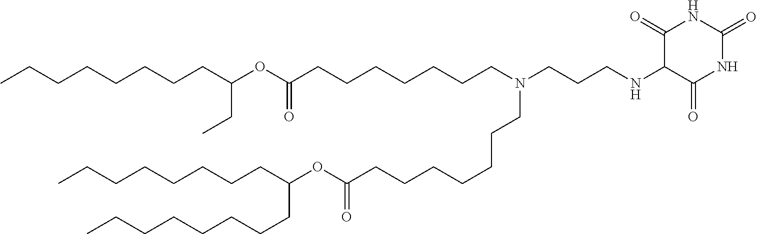 [SMILES: CCCCCCCCC(CC)OC(=O)CCCCCCCN(CCCCCCCC(=O)OC(CCCCCCCC)CCCCCCCC)CCCNC1C(=O)NC(=O)NC1=O] |
| V-19 | 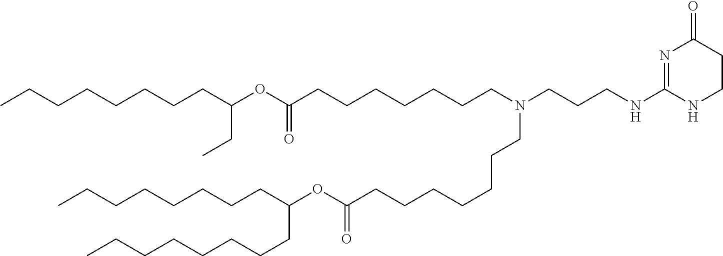 [SMILES: CCCCCCCCC(CC)OC(=O)CCCCCCCN(CCCCCCCC(=O)OC(CCCCCCCC)CCCCCCCC)CCCNC1=NC(=O)CCN1] |
| V-20 | 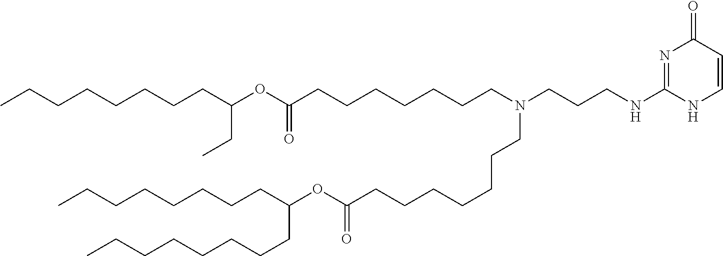 [SMILES: CCCCCCCCC(CC)OC(=O)CCCCCCCN(CCCCCCCC(=O)OC(CCCCCCCC)CCCCCCCC)CCCNc1nc(=O)cc[nH]1] |
| V-21 | 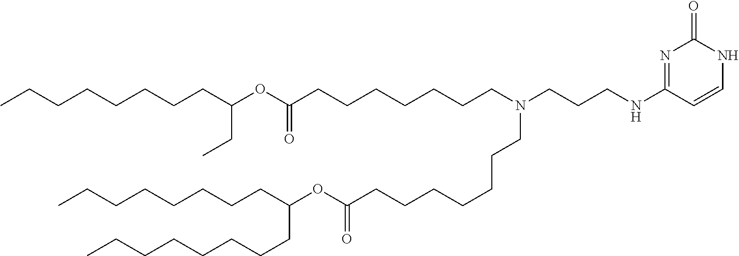 [SMILES: CCCCCCCCC(CC)OC(=O)CCCCCCCN(CCCCCCCC(=O)OC(CCCCCCCC)CCCCCCCC)CCCNc1cc[nH]c(=O)n1] |
| V-22 | 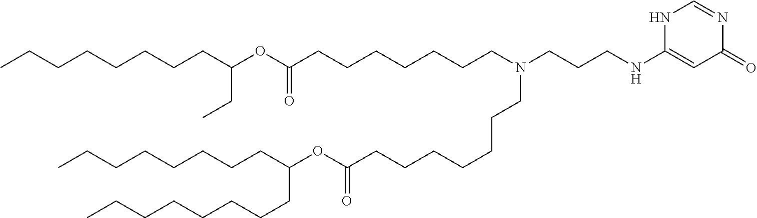 [SMILES: CCCCCCCCC(CC)OC(=O)CCCCCCCN(CCCCCCCC(=O)OC(CCCCCCCC)CCCCCCCC)CCCNc1cc(=O)nc[nH]1] |
| V-23 | 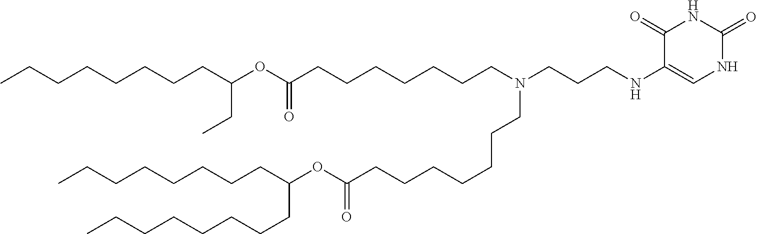 [SMILES: CCCCCCCCC(CC)OC(=O)CCCCCCCN(CCCCCCCC(=O)OC(CCCCCCCC)CCCCCCCC)CCCNc1c[nH]c(=O)[nH]c1=O] |
| V-24 | 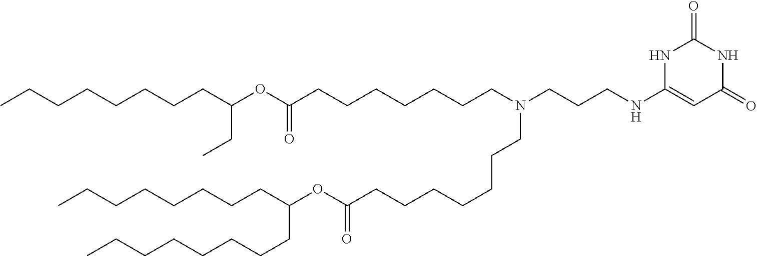 [SMILES: CCCCCCCCC(CC)OC(=O)CCCCCCCN(CCCCCCCC(=O)OC(CCCCCCCC)CCCCCCCC)CCCNc1cc(=O)[nH]c(=O)[nH]1] |
| V-25 | 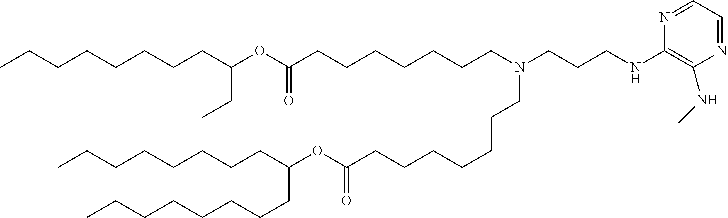 [SMILES: CCCCCCCCC(CC)OC(=O)CCCCCCCN(CCCCCCCC(=O)OC(CCCCCCCC)CCCCCCCC)CCCNc1nccnc1NC] |
| V-26 | 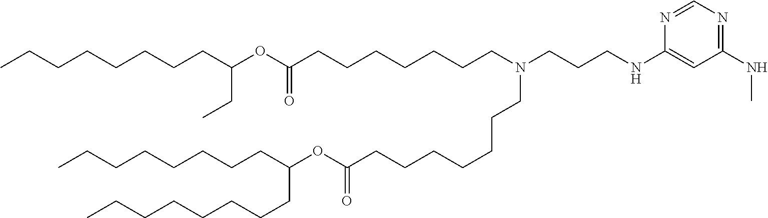 [SMILES: CCCCCCCCC(CC)OC(=O)CCCCCCCN(CCCCCCCC(=O)OC(CCCCCCCC)CCCCCCCC)CCCNc1cc(NC)ncn1] |
| V-27 | 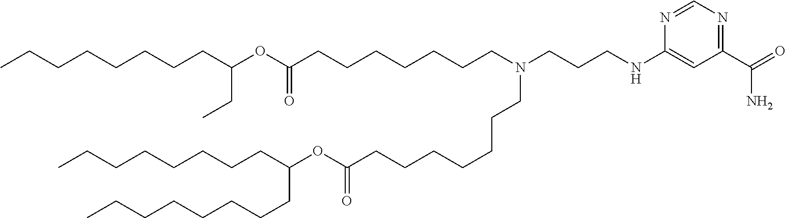 [SMILES: CCCCCCCCC(CC)OC(=O)CCCCCCCN(CCCCCCCC(=O)OC(CCCCCCCC)CCCCCCCC)CCCNc1cc(C(N)=O)ncn1] |
| V-28 | 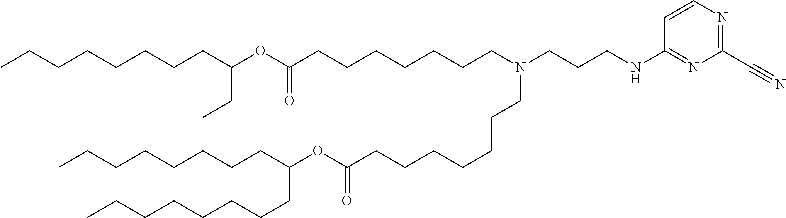 [SMILES: CCCCCCCCC(CC)OC(=O)CCCCCCCN(CCCCCCCC(=O)OC(CCCCCCCC)CCCCCCCC)CCCNc1ccnc(C#N)n1] |
| V-29 | 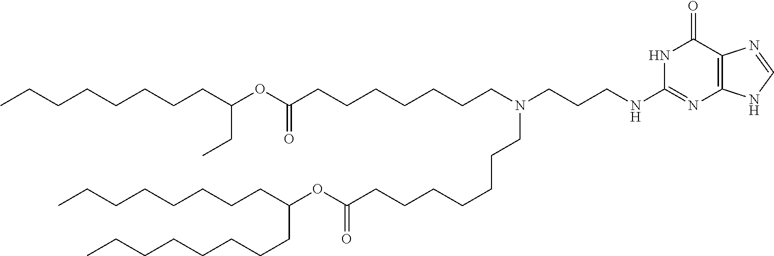 [SMILES: CCCCCCCCC(CC)OC(=O)CCCCCCCN(CCCCCCCC(=O)OC(CCCCCCCC)CCCCCCCC)CCCNc1nc2[nH]cnc2c(=O)[nH]1] |
| V-30 | 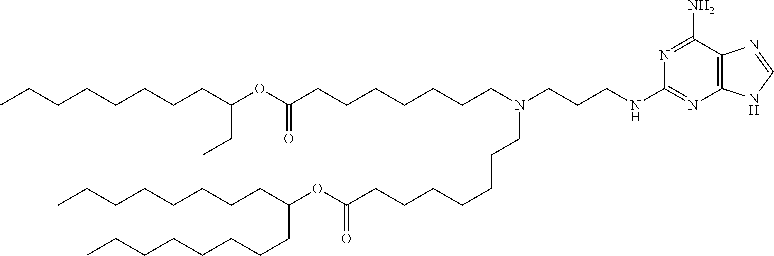 [SMILES: CCCCCCCCC(CC)OC(=O)CCCCCCCN(CCCCCCCC(=O)OC(CCCCCCCC)CCCCCCCC)CCCNc1nc(N)c2nc[nH]c2n1] |
| V-31 | 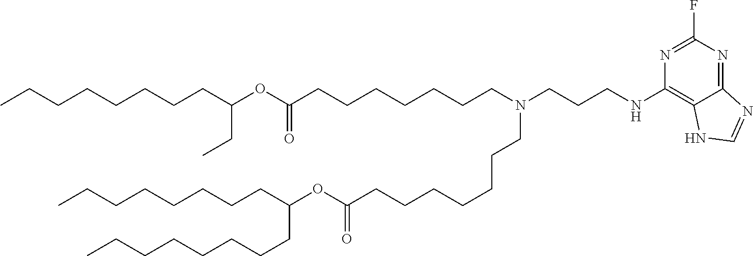 [SMILES: CCCCCCCCC(CC)OC(=O)CCCCCCCN(CCCCCCCC(=O)OC(CCCCCCCC)CCCCCCCC)CCCNc1nc(F)nc2nc[nH]c12] |
| V-32 | 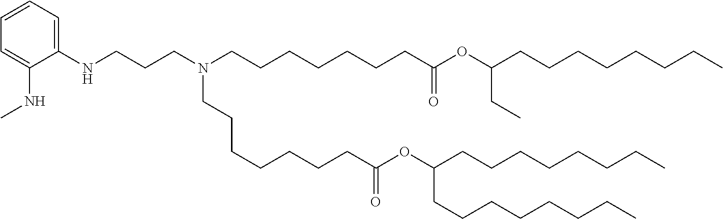 [SMILES: CCCCCCCCC(CC)OC(=O)CCCCCCCN(CCCCCCCC(=O)OC(CCCCCCCC)CCCCCCCC)CCCNc1ccccc1NC] |
| V-33 | 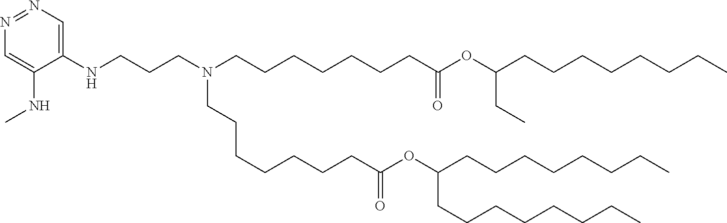 [SMILES: CCCCCCCCC(CC)OC(=O)CCCCCCCN(CCCCCCCC(=O)OC(CCCCCCCC)CCCCCCCC)CCCNc1cnncc1NC] |
| V-34 | 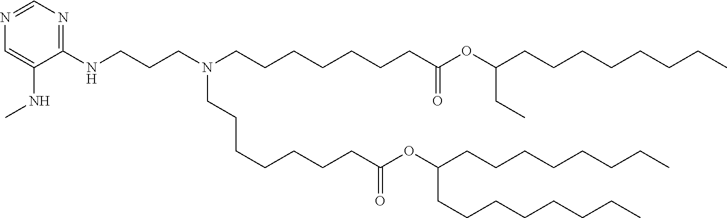 [SMILES: CCCCCCCCC(CC)OC(=O)CCCCCCCN(CCCCCCCC(=O)OC(CCCCCCCC)CCCCCCCC)CCCNc1ncncc1NC] |
| V-35 | 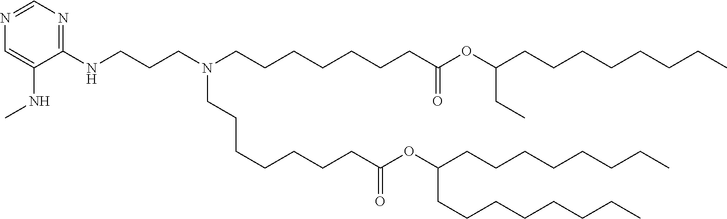 [SMILES: CCCCCCCCC(CC)OC(=O)CCCCCCCN(CCCCCCCC(=O)OC(CCCCCCCC)CCCCCCCC)CCCNc1ncncc1NC] |
| V-36 | 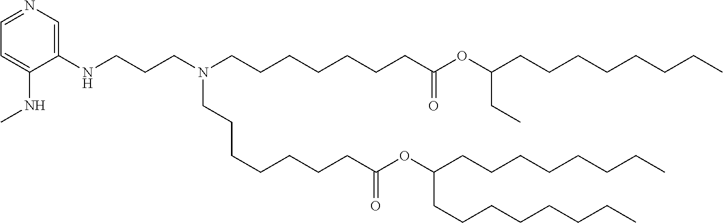 [SMILES: CCCCCCCCC(CC)OC(=O)CCCCCCCN(CCCCCCCC(=O)OC(CCCCCCCC)CCCCCCCC)CCCNc1cnccc1NC] |
| V-37 | 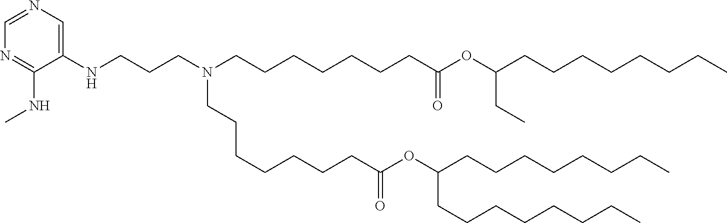 [SMILES: CCCCCCCCC(CC)OC(=O)CCCCCCCN(CCCCCCCC(=O)OC(CCCCCCCC)CCCCCCCC)CCCNc1cncnc1NC] |
| V-38 | 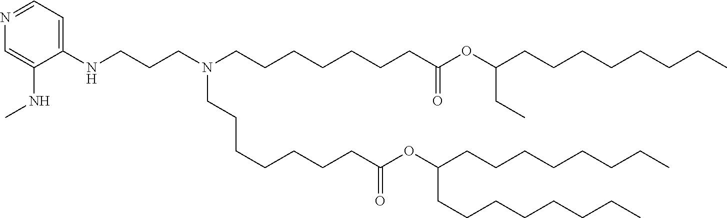 [SMILES: CCCCCCCCC(CC)OC(=O)CCCCCCCN(CCCCCCCC(=O)OC(CCCCCCCC)CCCCCCCC)CCCNc1ccncc1NC] |
| V-39 | 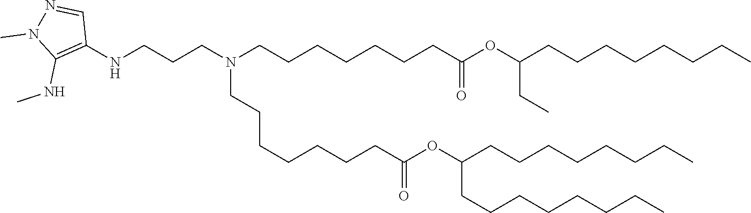 [SMILES: CCCCCCCCC(CC)OC(=O)CCCCCCCN(CCCCCCCC(=O)OC(CCCCCCCC)CCCCCCCC)CCCNc1cnn(C)c1NC] |
| V-40 | 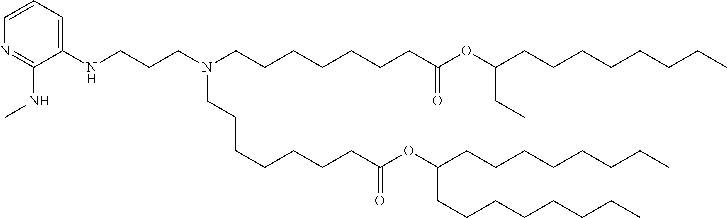 [SMILES: CCCCCCCCC(CC)OC(=O)CCCCCCCN(CCCCCCCC(=O)OC(CCCCCCCC)CCCCCCCC)CCCNc1cccnc1NC] |
| V-41 | 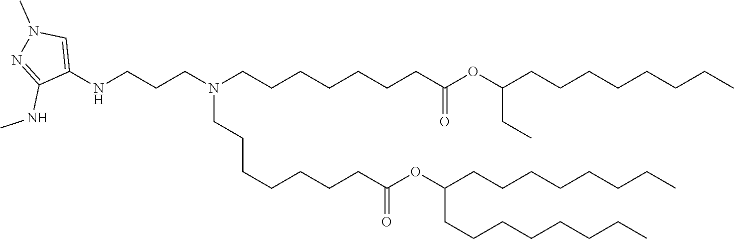 [SMILES: CCCCCCCCC(CC)OC(=O)CCCCCCCN(CCCCCCCC(=O)OC(CCCCCCCC)CCCCCCCC)CCCNc1cn(C)nc1NC] |
| V-42 | 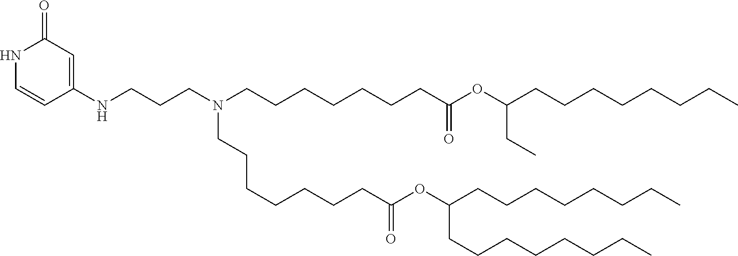 [SMILES: CCCCCCCCC(CC)OC(=O)CCCCCCCN(CCCCCCCC(=O)OC(CCCCCCCC)CCCCCCCC)CCCNc1cc[nH]c(=O)c1] |
| V-43 | 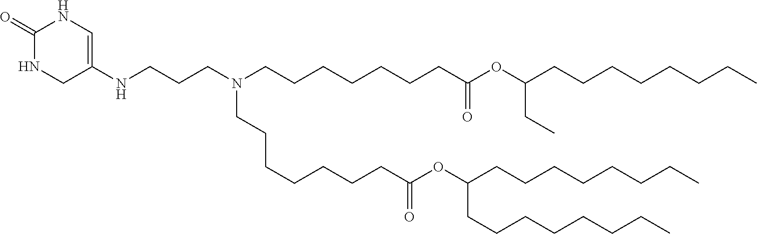 [SMILES: CCCCCCCCC(CC)OC(=O)CCCCCCCN(CCCCCCCC(=O)OC(CCCCCCCC)CCCCCCCC)CCCNC1=CNC(=O)NC1] |
| V-44 | 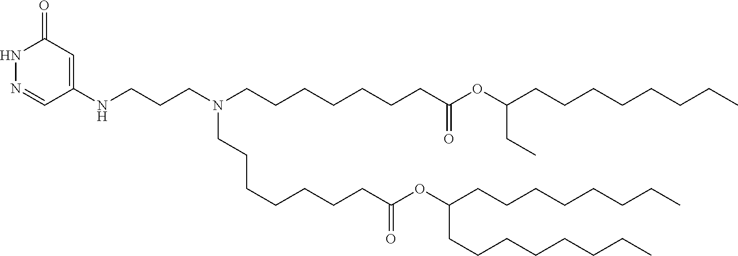 [SMILES: CCCCCCCCC(CC)OC(=O)CCCCCCCN(CCCCCCCC(=O)OC(CCCCCCCC)CCCCCCCC)CCCNc1cn[nH]c(=O)c1] |
| V-45 | 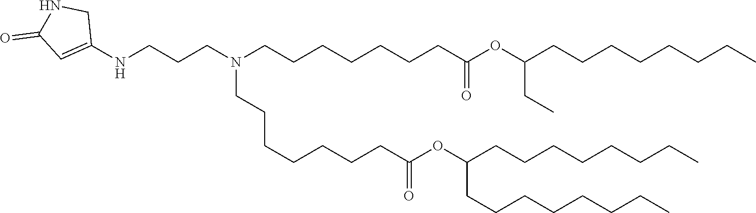 [SMILES: CCCCCCCCC(CC)OC(=O)CCCCCCCN(CCCCCCCC(=O)OC(CCCCCCCC)CCCCCCCC)CCCNC1=CC(=O)NC1] |
| V-46 | 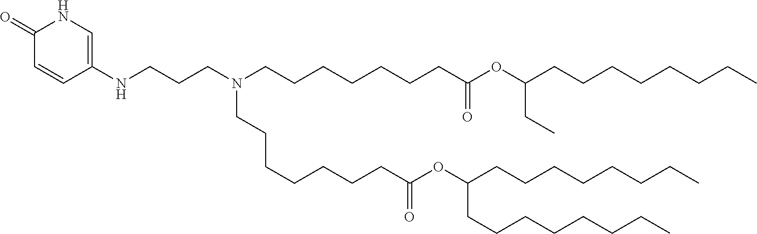 [SMILES: CCCCCCCCC(CC)OC(=O)CCCCCCCN(CCCCCCCC(=O)OC(CCCCCCCC)CCCCCCCC)CCCNc1ccc(=O)[nH]c1] |
| V-47 | 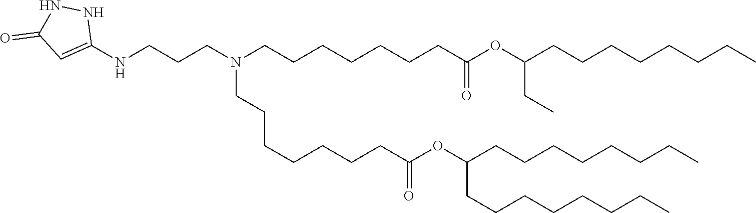 [SMILES: CCCCCCCCC(CC)OC(=O)CCCCCCCN(CCCCCCCC(=O)OC(CCCCCCCC)CCCCCCCC)CCCNc1cc(=O)[nH][nH]1] |
| V-48 | 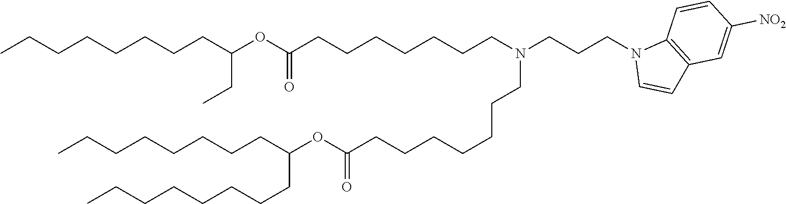 [SMILES: CCCCCCCCC(CC)OC(=O)CCCCCCCN(CCCCCCCC(=O)OC(CCCCCCCC)CCCCCCCC)CCCn1ccc2cc([N+](=O)[O-])ccc21] |
| V-49 | 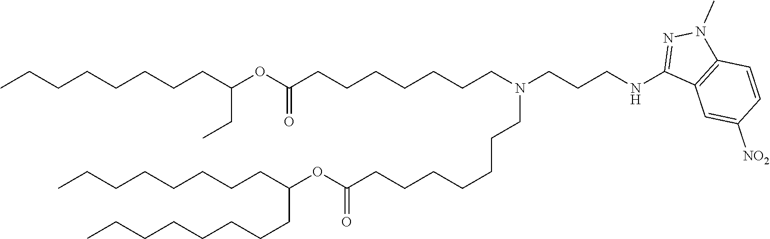 [SMILES: CCCCCCCCC(CC)OC(=O)CCCCCCCN(CCCCCCCC(=O)OC(CCCCCCCC)CCCCCCCC)CCCNc1nn(C)c2ccc([N+](=O)[O-])cc12] |
| V-50 | 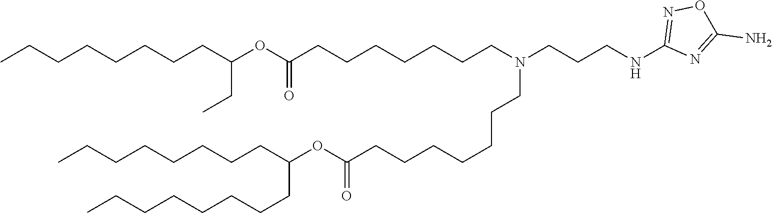 [SMILES: CCCCCCCCC(CC)OC(=O)CCCCCCCN(CCCCCCCC(=O)OC(CCCCCCCC)CCCCCCCC)CCCNc1noc(N)n1] |
| V-51 | 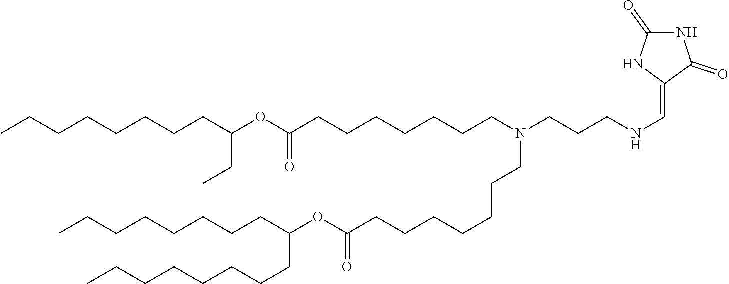 [SMILES: CCCCCCCCC(CC)OC(=O)CCCCCCCN(CCCCCCCC(=O)OC(CCCCCCCC)CCCCCCCC)CCCN/C=C1\NC(=O)NC1=O] |
| V-52 | 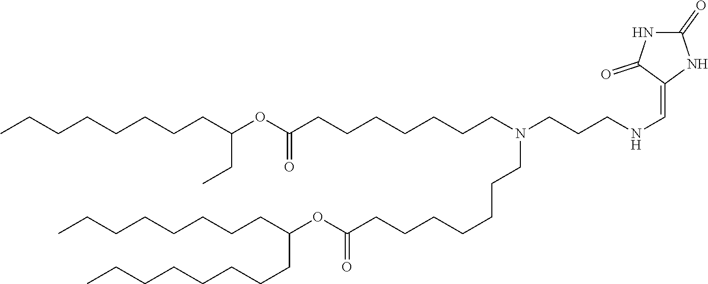 [SMILES: CCCCCCCCC(CC)OC(=O)CCCCCCCN(CCCCCCCC(=O)OC(CCCCCCCC)CCCCCCCC)CCCN/C=C1/NC(=O)NC1=O] |
| V-53 | 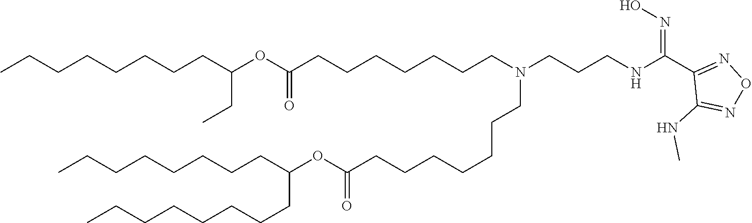 [SMILES: CCCCCCCCC(CC)OC(=O)CCCCCCCN(CCCCCCCC(=O)OC(CCCCCCCC)CCCCCCCC)CCCN/C(=N\O)c1nonc1NC] |
| V-54 | 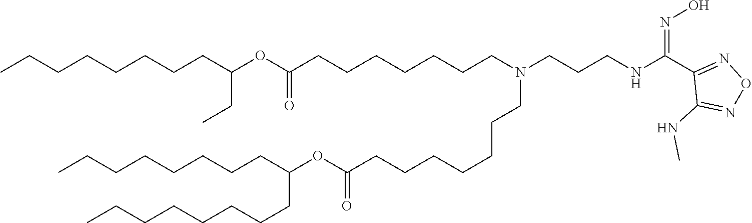 [SMILES: CCCCCCCCC(CC)OC(=O)CCCCCCCN(CCCCCCCC(=O)OC(CCCCCCCC)CCCCCCCC)CCCN/C(=N/O)c1nonc1NC] |
| V-55 | 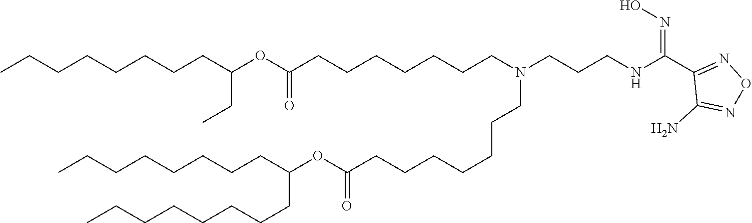 [SMILES: CCCCCCCCC(CC)OC(=O)CCCCCCCN(CCCCCCCC(=O)OC(CCCCCCCC)CCCCCCCC)CCCN/C(=N\O)c1nonc1N] |
| V-56 | 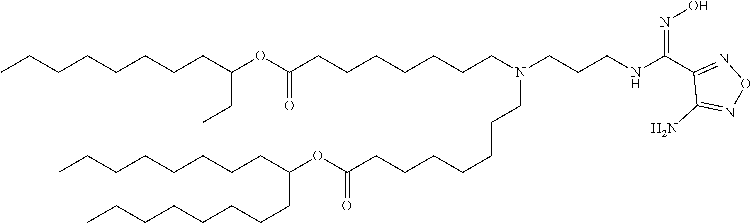 [SMILES: CCCCCCCCC(CC)OC(=O)CCCCCCCN(CCCCCCCC(=O)OC(CCCCCCCC)CCCCCCCC)CCCN/C(=N/O)c1nonc1N] |
| V-57 | 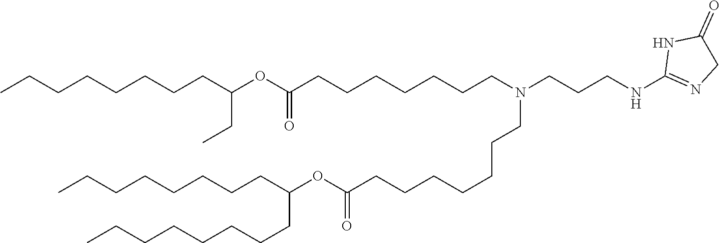 [SMILES: CCCCCCCCC(CC)OC(=O)CCCCCCCN(CCCCCCCC(=O)OC(CCCCCCCC)CCCCCCCC)CCCNC1=NCC(=O)N1] |

In some aspects, the ionizable lipid is a compound of Formula (IL-III):

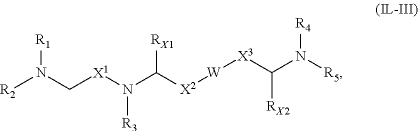

or salts or isomers thereof, wherein,
W is

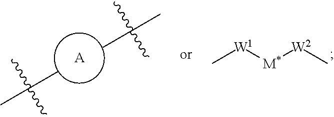

ring A is

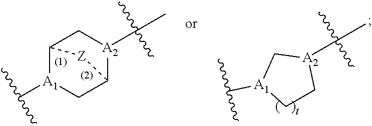

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;
M* is C₁-C₆ alkyl, W¹ and W² are each independently selected from the group consisting of —O— and —N(R₆)—;
each R₆ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6.

In some aspects, the ionizable lipid is a compound of Formula (IL-IIIA):

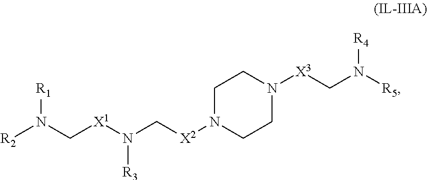

or a salt or isomer thereof, wherein
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
X¹, X², and X³ are independently selected from the group consisting of a bond, —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H; and
each R″ is independently selected from the group consisting of C₃₋₁₂ alkyl and C₃₋₁₂ alkenyl.

In some embodiments, the ionizable lipid is a compound selected from Table IL-7.

TABLE IL-7
Ionizable lipids

| Cpd. | |
|---|---|
| # | Structure |
| VI-1 | 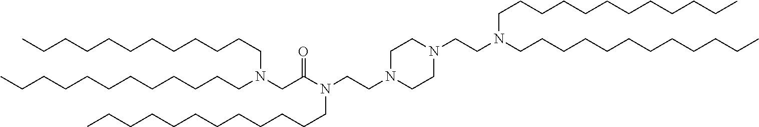 [SMILES: CCCCCCCCCCCCN(CCCCCCCCCCCC)CCN1CCN(CCN(CCCCCCCCCCCC)C(=O)CN(CCCCCCCCCCCC)CCCCCCCCCCCC)CC1] |
| VI-2 | 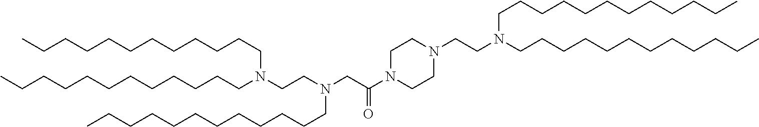 [SMILES: CCCCCCCCCCCCN(CCCCCCCCCCCC)CCN1CCN(C(=O)CN(CCCCCCCCCCCC)CCN(CCCCCCCCCCCC)CCCCCCCCCCCC)CC1] |
| VI-3 | 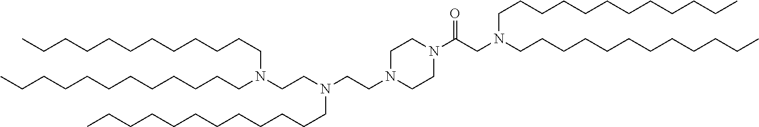 [SMILES: CCCCCCCCCCCCN(CCCCCCCCCCCC)CCN(CCCCCCCCCCCC)CCN1CCN(C(=O)CN(CCCCCCCCCCCC)CCCCCCCCCCCC)CC1] |
| VI-4 | 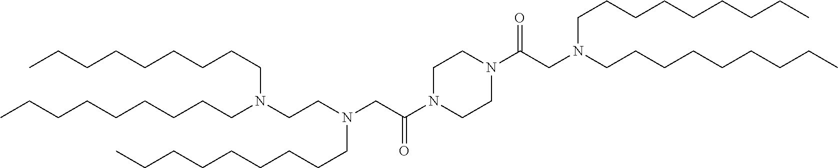 [SMILES: CCCCCCCCCN(CCCCCCCCC)CCN(CCCCCCCCC)CC(=O)N1CCN(C(=O)CN(CCCCCCCCC)CCCCCCCCC)CC1] |
| VI-5 | 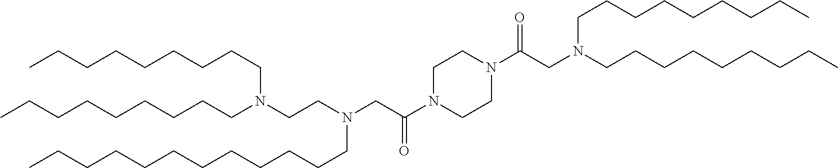 [SMILES: CCCCCCCCCCCCN(CCN(CCCCCCCCC)CCCCCCCCC)CC(=O)N1CCN(C(=O)CN(CCCCCCCCC)CCCCCCCCC)CC1] |
| VI-6 | 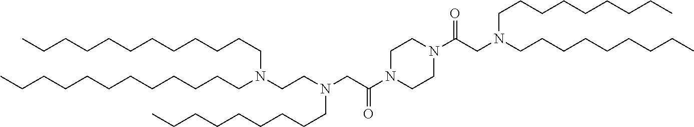 [SMILES: CCCCCCCCCCCCN(CCCCCCCCCCCC)CCN(CCCCCCCCC)CC(=O)N1CCN(C(=O)CN(CCCCCCCCC)CCCCCCCCC)CC1] |
| VI-7 | 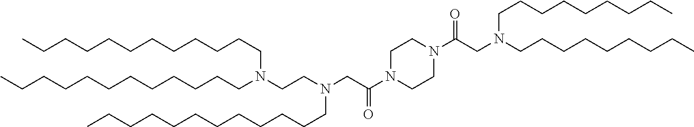 [SMILES: CCCCCCCCCCCCN(CCCCCCCCCCCC)CCN(CCCCCCCCCCCC)CC(=O)N1CCN(C(=O)CN(CCCCCCCCC)CCCCCCCCC)CC1] |
| VI-8 | 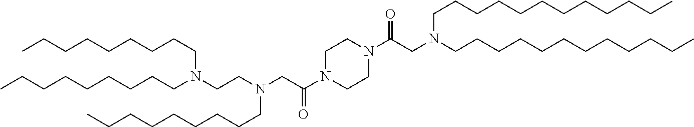 [SMILES: CCCCCCCCCCCCN(CCCCCCCCCCCC)CC(=O)N1CCN(C(=O)CN(CCCCCCCCC)CCN(CCCCCCCCC)CCCCCCCCC)CC1] |
| VI-9 | 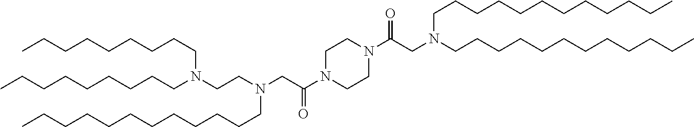 [SMILES: CCCCCCCCCCCCN(CCCCCCCCCCCC)CC(=O)N1CCN(C(=O)CN(CCCCCCCCCCCC)CCN(CCCCCCCCC)CCCCCCCCC)CC1] |
| VI-10 | 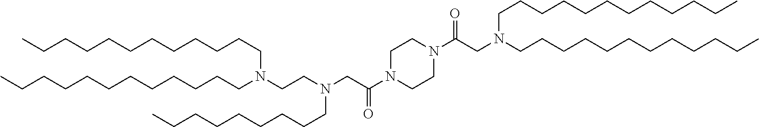 [SMILES: CCCCCCCCCCCCN(CCCCCCCCCCCC)CCN(CCCCCCCCC)CC(=O)N1CCN(C(=O)CN(CCCCCCCCCCCC)CCCCCCCCCCCC)CC1] |
| VI-11 | 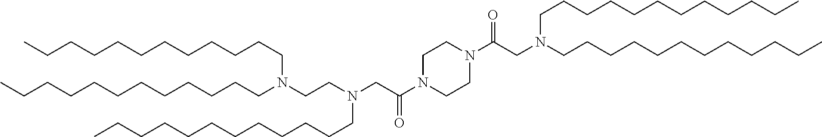 [SMILES: CCCCCCCCCCCCN(CCCCCCCCCCCC)CCN(CCCCCCCCCCCC)CC(=O)N1CCN(C(=O)CN(CCCCCCCCCCCC)CCCCCCCCCCCC)CC1] |
| VI-12 | 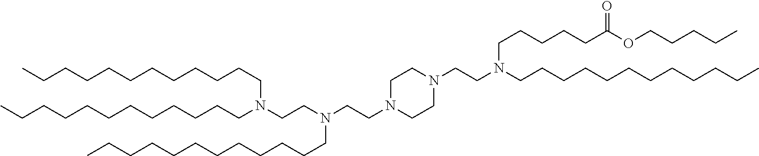 [SMILES: CCCCCCCCCCCCN(CCCCCCCCCCCC)CCN(CCCCCCCCCCCC)CCN1CCN(CCN(CCCCCCCCCCCC)CCCCCC(=O)OCCCCC)CC1] |
| VI-13 | 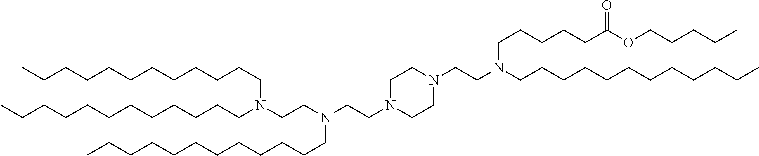 [SMILES: CCCCCCCCCCCCN(CCCCCCCCCCCC)CCN(CCCCCCCCCCCC)CCN1CCN(CCN(CCCCCCCCCCCC)CCCCCC(=O)OCCCCC)CC1] |
| VI-14 | 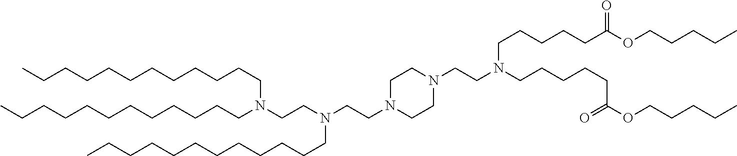 [SMILES: CCCCCCCCCCCCN(CCCCCCCCCCCC)CCN(CCCCCCCCCCCC)CCN1CCN(CCN(CCCCCC(=O)OCCCCC)CCCCCC(=O)OCCCCC)CC1] |
| VI-15 | 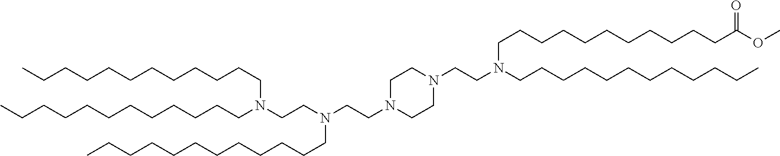 [SMILES: CCCCCCCCCCCCN(CCCCCCCCCCCC)CCN(CCCCCCCCCCCC)CCN1CCN(CCN(CCCCCCCCCCCC)CCCCCCCCCCCC(=O)OC)CC1] |
| VI-16 | 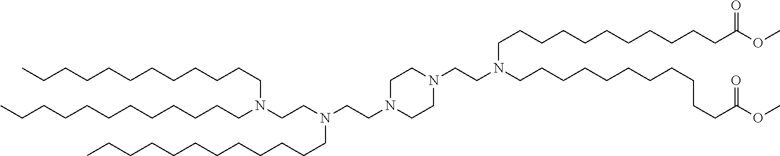 [SMILES: CCCCCCCCCCCCN(CCCCCCCCCCCC)CCN(CCCCCCCCCCCC)CCN1CCN(CCN(CCCCCCCCCCCC(=O)OC)CCCCCCCCCCCC(=O)OC)CC1] |
| VI-17 | 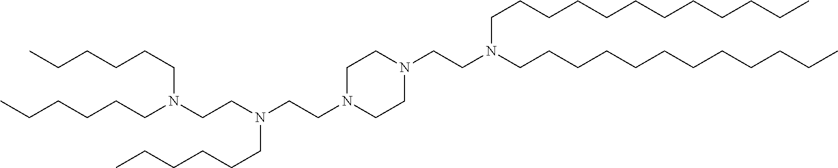 [SMILES: CCCCCCCCCCCCN(CCCCCCCCCCCC)CCN1CCN(CCN(CCCCCC)CCN(CCCCCC)CCCCCC)CC1] |
| VI-18 | 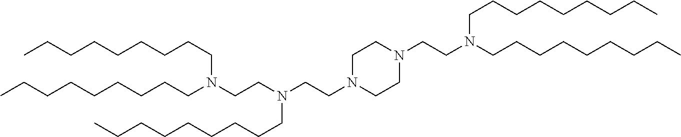 [SMILES: CCCCCCCCCN(CCCCCCCCC)CCN(CCCCCCCCC)CCN1CCN(CCN(CCCCCCCCC)CCCCCCCCC)CC1] |
| VI-19 | 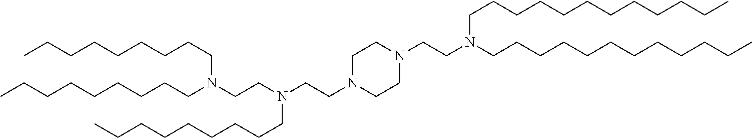 [SMILES: CCCCCCCCCCCCN(CCCCCCCCCCCC)CCN1CCN(CCN(CCCCCCCCC)CCN(CCCCCCCCC)CCCCCCCCC)CC1] |
| VI-20 | 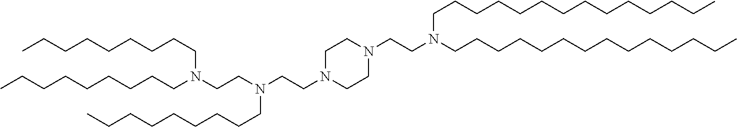 [SMILES: CCCCCCCCCCCCCCN(CCCCCCCCCCCCCC)CCN1CCN(CCN(CCCCCCCCC)CCN(CCCCCCCCC)CCCCCCCCC)CC1] |
| VI-21 | 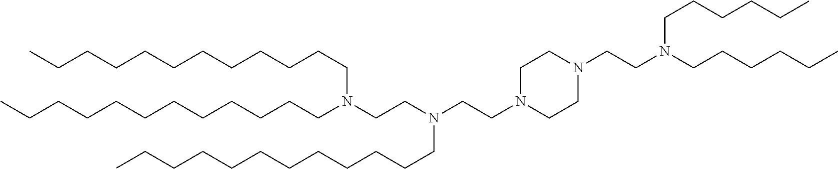 [SMILES: CCCCCCCCCCCCN(CCCCCCCCCCCC)CCN(CCCCCCCCCCCC)CCN1CCN(CCN(CCCCCC)CCCCCC)CC1] |
| VI-22 | 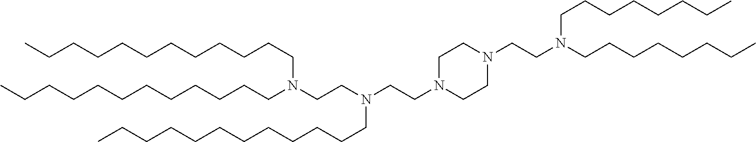 [SMILES: CCCCCCCCCCCCN(CCCCCCCCCCCC)CCN(CCCCCCCCCCCC)CCN1CCN(CCN(CCCCCCCC)CCCCCCCC)CC1] |
| VI-23 | 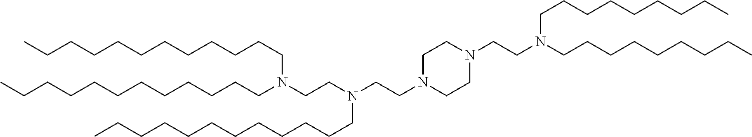 [SMILES: CCCCCCCCCCCCN(CCCCCCCCCCCC)CCN(CCCCCCCCCCCC)CCN1CCN(CCN(CCCCCCCCC)CCCCCCCCC)CC1] |
| VI-24 | 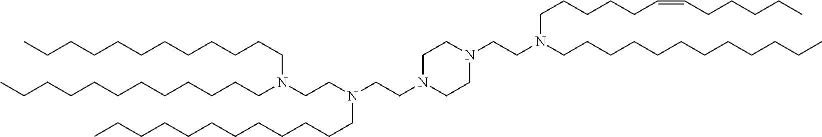 [SMILES: CCCCC/C=C\CCCCCN(CCCCCCCCCCCC)CCN1CCN(CCN(CCCCCCCCCCCC)CCN(CCCCCCCCCCCC)CCCCCCCCCCCC)CC1] |
| VI-25 | 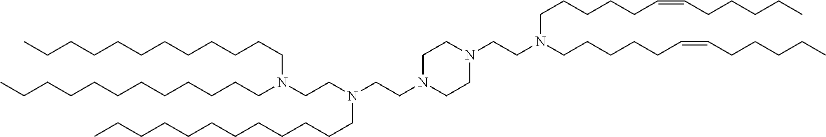 [SMILES: CCCCC/C=C\CCCCCN(CCCCC/C=C\CCCCC)CCN1CCN(CCN(CCCCCCCCCCCC)CCN(CCCCCCCCCCCC)CCCCCCCCCCCC)CC1] |
| VI-26 | 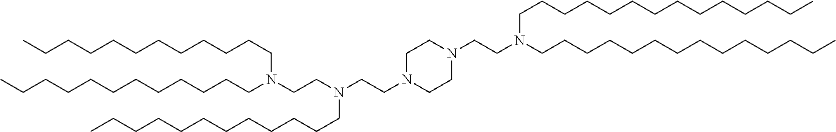 [SMILES: CCCCCCCCCCCCCCN(CCCCCCCCCCCCCC)CCN1CCN(CCN(CCCCCCCCCCCC)CCN(CCCCCCCCCCCC)CCCCCCCCCCCC)CC1] |
| VI-27 | 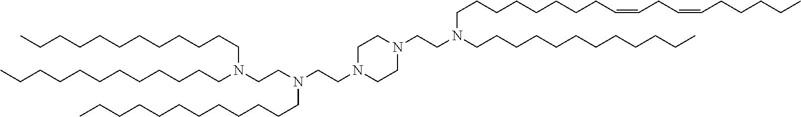 [SMILES: CCCCC/C=C\C/C=C\CCCCCCCCN(CCCCCCCCCCCC)CCN1CCN(CCN(CCCCCCCCCCCC)CCN(CCCCCCCCCCCC)CCCCCCCCCCCC)CC1] |
| VI-28 | 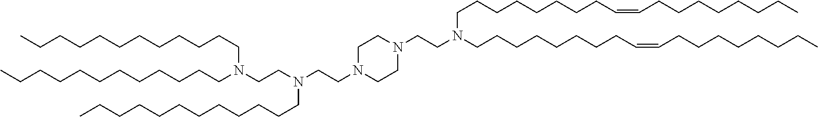 [SMILES: CCCCCCCC/C=C\CCCCCCCCN(CCCCCCCC/C=C\CCCCCCCC)CCN1CCN(CCN(CCCCCCCCCCCC)CCN(CCCCCCCCCCCC)CCCCCCCCCCCC)CC1] |
| VI-29 | 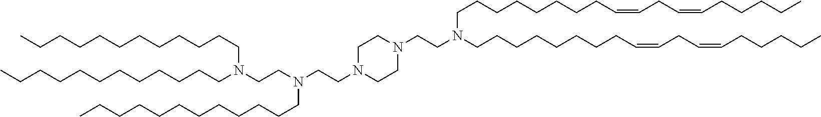 [SMILES: CCCCC/C=C\C/C=C\CCCCCCCCN(CCCCCCCC/C=C\C/C=C\CCCCC)CCN1CCN(CCN(CCCCCCCCCCCC)CCN(CCCCCCCCCCCC)CCCCCCCCCCCC)CC1] |
| VI-30 | 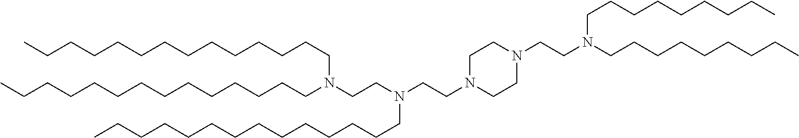 [SMILES: CCCCCCCCCCCCCCN(CCCCCCCCCCCCCC)CCN(CCCCCCCCCCCCCC)CCN1CCN(CCN(CCCCCCCCC)CCCCCCCCC)CC1] |
| VI-31 | 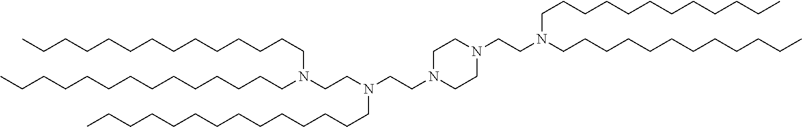 [SMILES: CCCCCCCCCCCCCCN(CCCCCCCCCCCCCC)CCN(CCCCCCCCCCCCCC)CCN1CCN(CCN(CCCCCCCCCCCC)CCCCCCCCCCCC)CC1] |
| VI-32 | 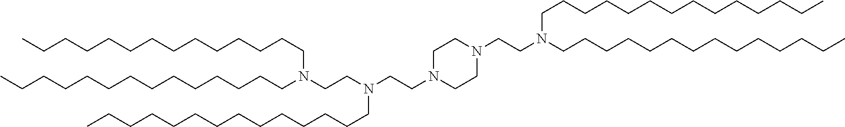 [SMILES: CCCCCCCCCCCCCCN(CCCCCCCCCCCCCC)CCN(CCCCCCCCCCCCCC)CCN1CCN(CCN(CCCCCCCCCCCCCC)CCCCCCCCCCCCCC)CC1] |
| VI-33 | 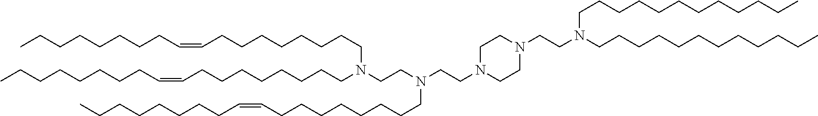 [SMILES: CCCCCCCC/C=C\CCCCCCCCN(CCCCCCCC/C=C\CCCCCCCC)CCN(CCCCCCCC/C=C\CCCCCCCC)CCN1CCN(CCN(CCCCCCCCCCCC)CCCCCCCCCCCC)CC1] |
| VI-34 | 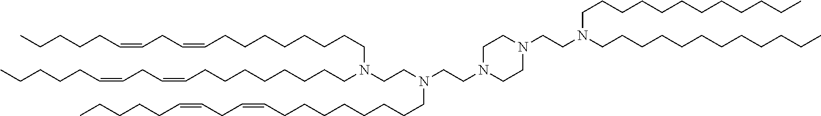 [SMILES: CCCCC/C=C\C/C=C\CCCCCCCCN(CCCCCCCC/C=C\C/C=C\CCCCC)CCN(CCCCCCCC/C=C\C/C=C\CCCCC)CCN1CCN(CCN(CCCCCCCCCCCC)CCCCCCCCCCCC)CC1] |
| VI-35 | 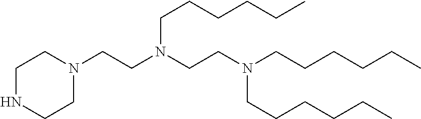 [SMILES: CCCCCCN(CCCCCC)CCN(CCCCCC)CCN1CCNCC1] |
| VI-36 | 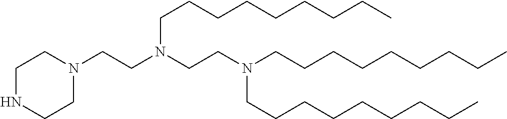 [SMILES: CCCCCCCCCN(CCCCCCCCC)CCN(CCCCCCCCC)CCN1CCNCC1] |
| VI-37 | 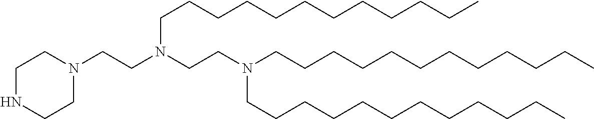 [SMILES: CCCCCCCCCCCCN(CCCCCCCCCCCC)CCN(CCCCCCCCCCCC)CCN1CCNCC1] |
| VI-38 | 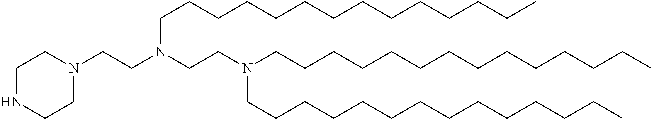 [SMILES: CCCCCCCCCCCCCCN(CCCCCCCCCCCCCC)CCN(CCCCCCCCCCCCCC)CCN1CCNCC1] |
| VI-39 | 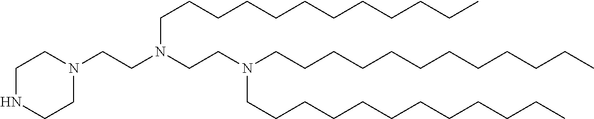 [SMILES: CCCCCCCCCCCCN(CCCCCCCCCCCC)CCN(CCCCCCCCCCCC)CCN1CCNCC1] |
| VI-40 | 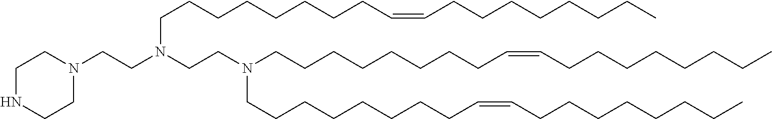 [SMILES: CCCCCCCC/C=C\CCCCCCCCN(CCCCCCCC/C=C\CCCCCCCC)CCN(CCCCCCCC/C=C\CCCCCCCC)CCN1CCNCC1] |
| VI-41 | 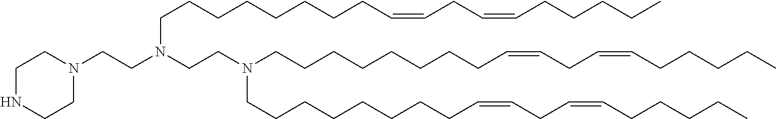 [SMILES: CCCCC/C=C\C/C=C\CCCCCCCCN(CCCCCCCC/C=C\C/C=C\CCCCC)CCN(CCCCCCCC/C=C\C/C=C\CCCCC)CCN1CCNCC1] |
| VI-IV | 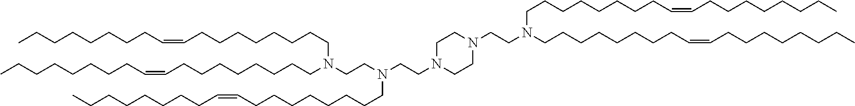 [SMILES: CCCCCCCC/C=C\CCCCCCCCN(CCCCCCCC/C=C\CCCCCCCC)CCN(CCCCCCCC/C=C\CCCCCCCC)CCN1CCN(CCN(CCCCCCCC/C=C\CCCCCCCC)CCCCCCCC/C=C\CCCCCCCC)CC1] |
| VI-42 | 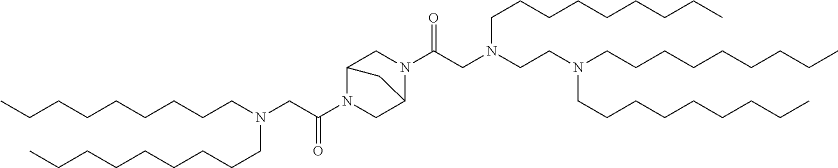 [SMILES: CCCCCCCCCN(CCCCCCCCC)CCN(CCCCCCCCC)CC(=O)N1CC2CC1CN2C(=O)CN(CCCCCCCCC)CCCCCCCCC] |
| VI-43 | 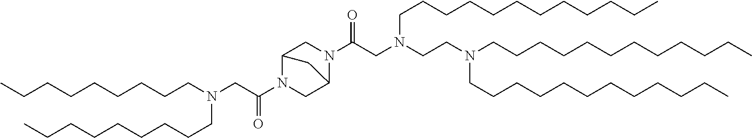 [SMILES: CCCCCCCCCCCCN(CCCCCCCCCCCC)CCN(CCCCCCCCCCCC)CC(=O)N1CC2CC1CN2C(=O)CN(CCCCCCCCC)CCCCCCCCC] |
| VI-44 | 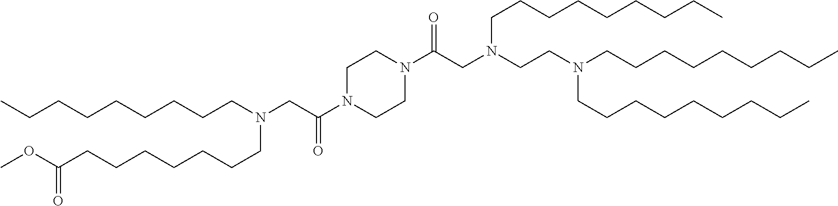 [SMILES: CCCCCCCCCN(CCCCCCCCC)CCN(CCCCCCCCC)CC(=O)N1CCN(C(=O)CN(CCCCCCCCC)CCCCCCCC(=O)OC)CC1] |
| VI-45 | 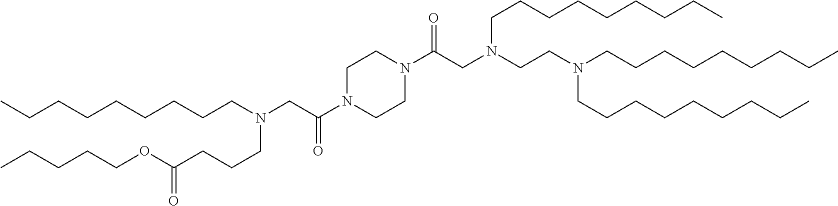 [SMILES: CCCCCCCCCN(CCCCCCCCC)CCN(CCCCCCCCC)CC(=O)N1CCN(C(=O)CN(CCCCCCCCC)CCCC(=O)OCCCCC)CC1] |
| VI-46 | 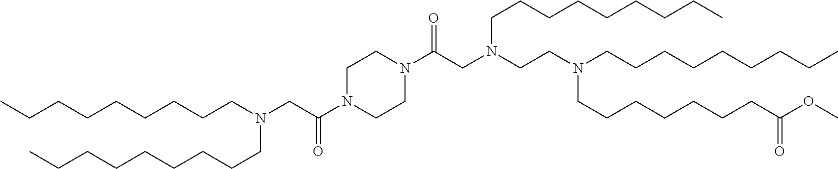 [SMILES: CCCCCCCCCN(CCCCCCCC(=O)OC)CCN(CCCCCCCCC)CC(=O)N1CCN(C(=O)CN(CCCCCCCCC)CCCCCCCCC)CC1] |
| VI-47 | 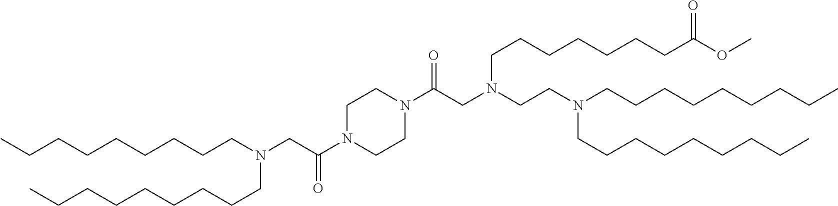 [SMILES: CCCCCCCCCN(CCCCCCCCC)CCN(CCCCCCCC(=O)OC)CC(=O)N1CCN(C(=O)CN(CCCCCCCCC)CCCCCCCCC)CC1] |
| VI-48 | 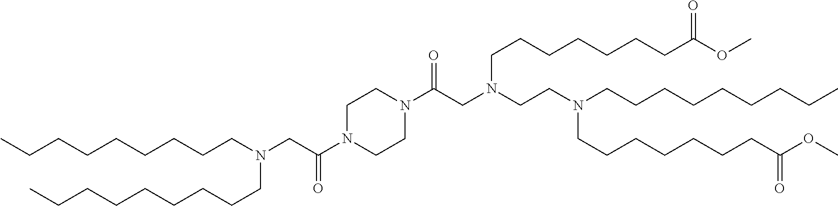 [SMILES: CCCCCCCCCN(CCCCCCCC(=O)OC)CCN(CCCCCCCC(=O)OC)CC(=O)N1CCN(C(=O)CN(CCCCCCCCC)CCCCCCCCC)CC1] |
| VI-49 | 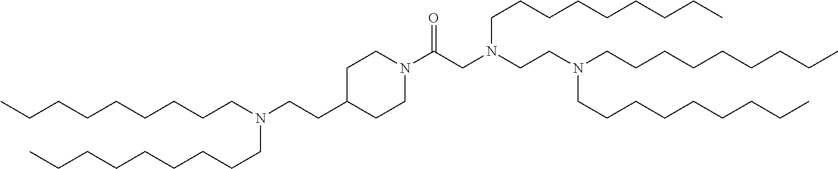 [SMILES: CCCCCCCCCN(CCCCCCCCC)CCC1CCN(C(=O)CN(CCCCCCCCC)CCN(CCCCCCCCC)CCCCCCCCC)CC1] |
| VI-50 | 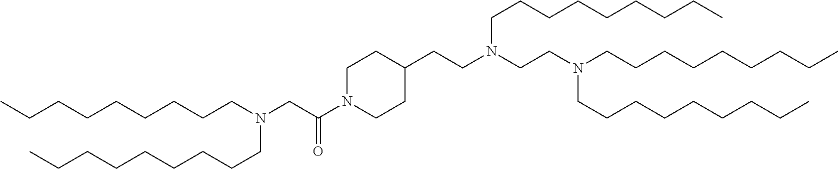 [SMILES: CCCCCCCCCN(CCCCCCCCC)CCN(CCCCCCCCC)CCC1CCN(C(=O)CN(CCCCCCCCC)CCCCCCCCC)CC1] |
| VI-51 | 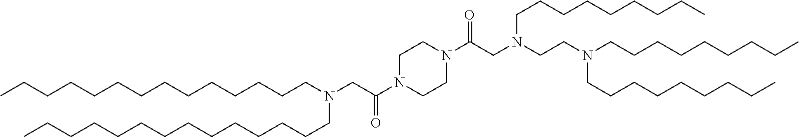 [SMILES: CCCCCCCCCCCCCCN(CCCCCCCCCCCCCC)CC(=O)N1CCN(C(=O)CN(CCCCCCCCC)CCN(CCCCCCCCC)CCCCCCCCC)CC1] |
| VI-52 | 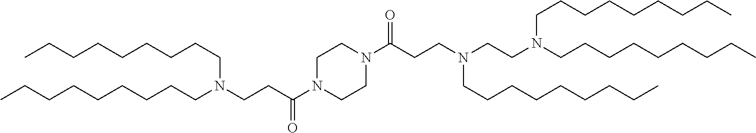 [SMILES: CCCCCCCCCN(CCCCCCCCC)CCC(=O)N1CCN(C(=O)CCN(CCCCCCCCC)CCN(CCCCCCCCC)CCCCCCCCC)CC1] |
| VI-53 | 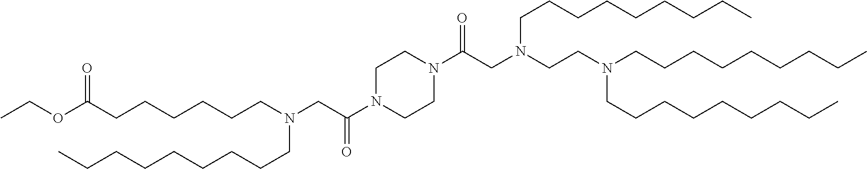 [SMILES: CCCCCCCCCN(CCCCCCCCC)CCN(CCCCCCCCC)CC(=O)N1CCN(C(=O)CN(CCCCCCCCC)CCCCCCC(=O)OCC)CC1] |
| VI-54 | 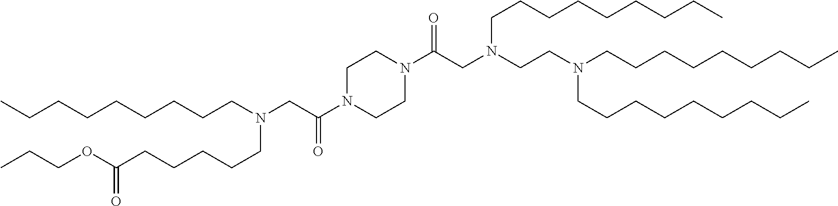 [SMILES: CCCCCCCCCN(CCCCCCCCC)CCN(CCCCCCCCC)CC(=O)N1CCN(C(=O)CN(CCCCCCCCC)CCCCCC(=O)OCCC)CC1] |
| VI-55 | 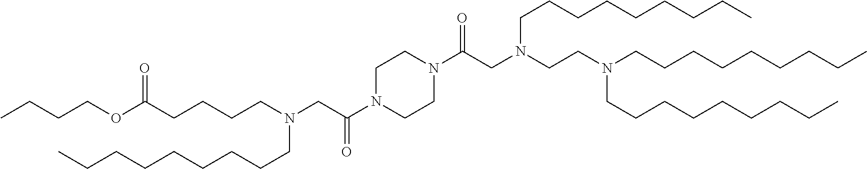 [SMILES: CCCCCCCCCN(CCCCCCCCC)CCN(CCCCCCCCC)CC(=O)N1CCN(C(=O)CN(CCCCCCCCC)CCCCC(=O)OCCCC)CC1] |
| VI-56 | 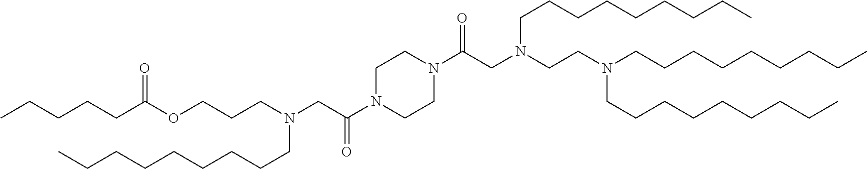 [SMILES: CCCCCCCCCN(CCCCCCCCC)CCN(CCCCCCCCC)CC(=O)N1CCN(C(=O)CN(CCCCCCCCC)CCCOC(=O)CCCCC)CC1] |
| VI-57 | 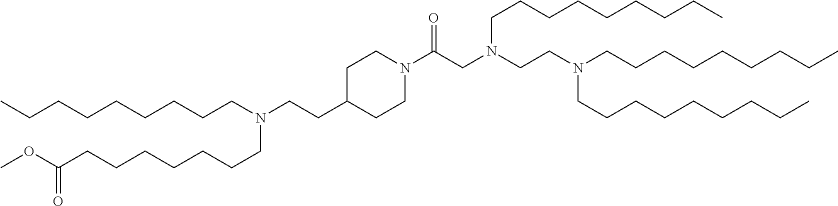 [SMILES: CCCCCCCCCN(CCCCCCCC(=O)OC)CCC1CCN(C(=O)CN(CCCCCCCCC)CCN(CCCCCCCCC)CCCCCCCCC)CC1] |
| VI-58 | 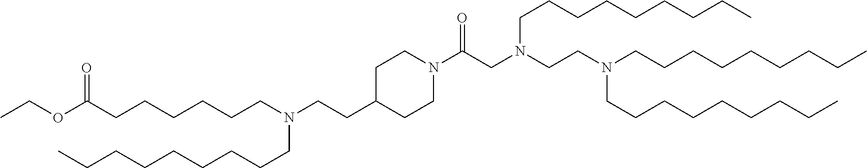 [SMILES: CCCCCCCCCN(CCCCCCC(=O)OCC)CCC1CCN(C(=O)CN(CCCCCCCCC)CCN(CCCCCCCCC)CCCCCCCCC)CC1] |
| VI-59 | 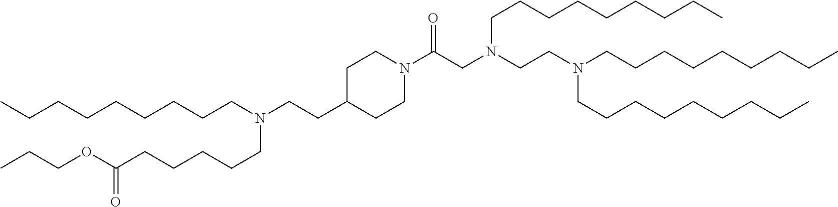 [SMILES: CCCCCCCCCN(CCCCCC(=O)OCCC)CCC1CCN(C(=O)CN(CCCCCCCCC)CCN(CCCCCCCCC)CCCCCCCCC)CC1] |
| VI-60 | 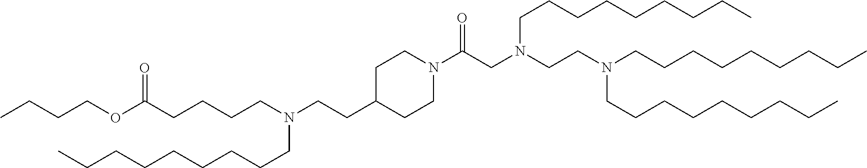 [SMILES: CCCCCCCCCN(CCCCC(=O)OCCCC)CCC1CCN(C(=O)CN(CCCCCCCCC)CCN(CCCCCCCCC)CCCCCCCCC)CC1] |
| VI-61 | 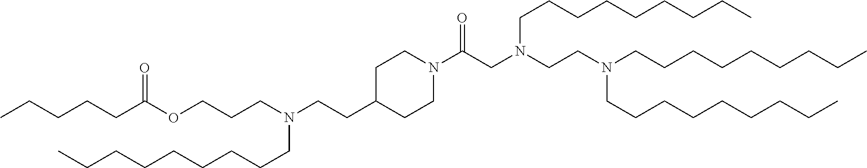 [SMILES: CCCCCCCCCN(CCCOC(=O)CCCCC)CCC1CCN(C(=O)CN(CCCCCCCCC)CCN(CCCCCCCCC)CCCCCCCCC)CC1] |
| VI-62 | 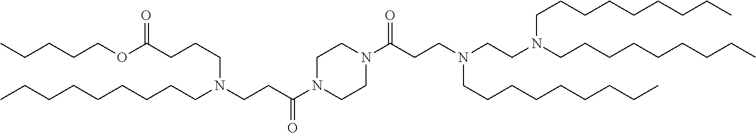 [SMILES: CCCCCCCCCN(CCCC(=O)OCCCCC)CCC(=O)N1CCN(C(=O)CCN(CCCCCCCCC)CCN(CCCCCCCCC)CCCCCCCCC)CC1] |
| VI-63 | 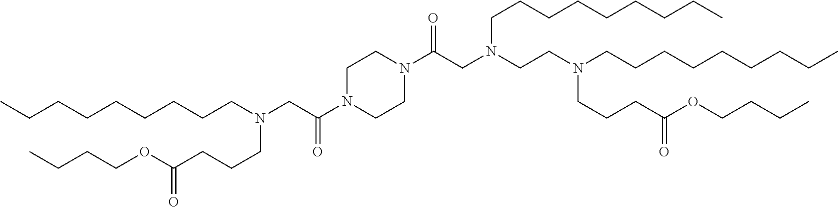 [SMILES: CCCCCCCCCN(CCCC(=O)OCCCC)CCN(CCCCCCCCC)CC(=O)N1CCN(C(=O)CN(CCCCCCCCC)CCCC(=O)OCCCC)CC1] |
| VI-64 | 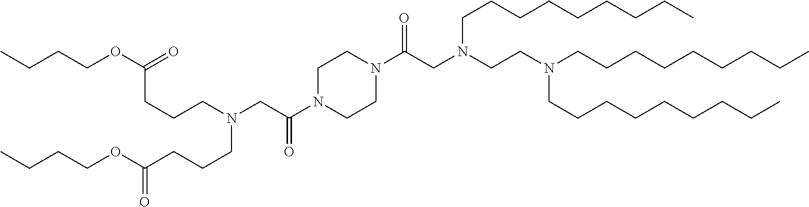 [SMILES: CCCCCCCCCN(CCCCCCCCC)CCN(CCCCCCCCC)CC(=O)N1CCN(C(=O)CN(CCCC(=O)OCCCC)CCCC(=O)OCCCC)CC1] |
| VI-65 | 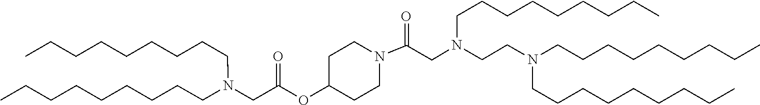 [SMILES: CCCCCCCCCN(CCCCCCCCC)CCN(CCCCCCCCC)CC(=O)N1CCC(OC(=O)CN(CCCCCCCCC)CCCCCCCCC)CC1] |
| VI-66 | 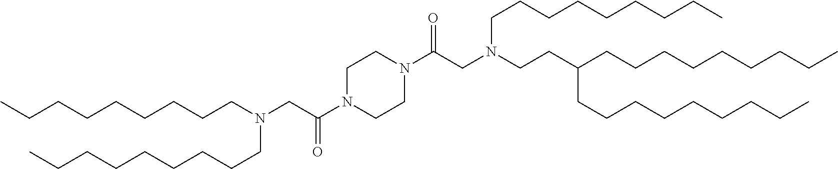 [SMILES: CCCCCCCCCC(CCCCCCCCC)CCN(CCCCCCCCC)CC(=O)N1CCN(C(=O)CN(CCCCCCCCC)CCCCCCCCC)CC1] |
| VI-67 | 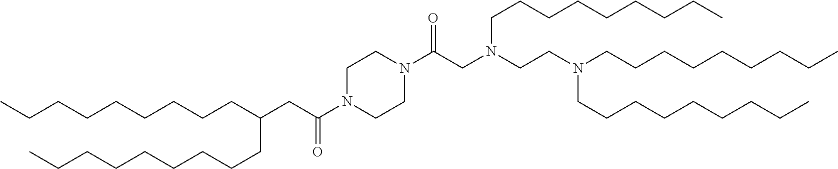 [SMILES: CCCCCCCCCC(CCCCCCCCC)CC(=O)N1CCN(C(=O)CN(CCCCCCCCC)CCN(CCCCCCCCC)CCCCCCCCC)CC1] |
| VI-68 | 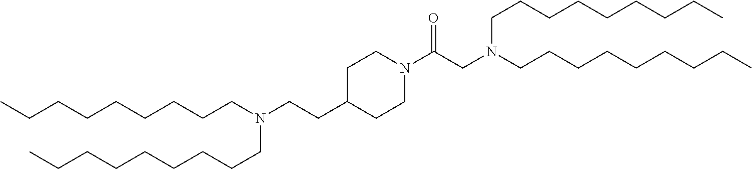 [SMILES: CCCCCCCCCN(CCCCCCCCC)CCC1CCN(C(=O)CN(CCCCCCCCC)CCCCCCCCC)CC1] |
| VI-69 | 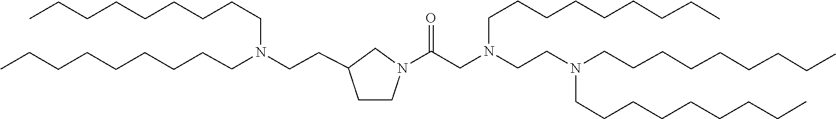 [SMILES: CCCCCCCCCN(CCCCCCCCC)CCC1CCN(C(=O)CN(CCCCCCCCC)CCN(CCCCCCCCC)CCCCCCCCC)C1] |
| VI-70 | 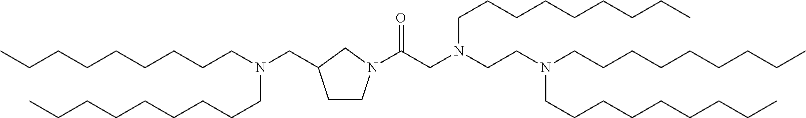 [SMILES: CCCCCCCCCN(CCCCCCCCC)CCN(CCCCCCCCC)CC(=O)N1CCC(CN(CCCCCCCCC)CCCCCCCCC)C1] |
| VI-71 | 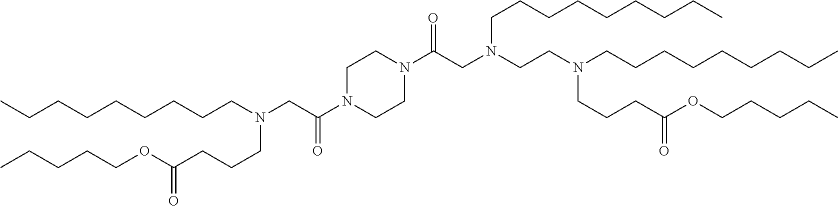 [SMILES: CCCCCCCCCN(CCCC(=O)OCCCCC)CCN(CCCCCCCCC)CC(=O)N1CCN(C(=O)CN(CCCCCCCCC)CCCC(=O)OCCCCC)CC1] |
| VI-72 | 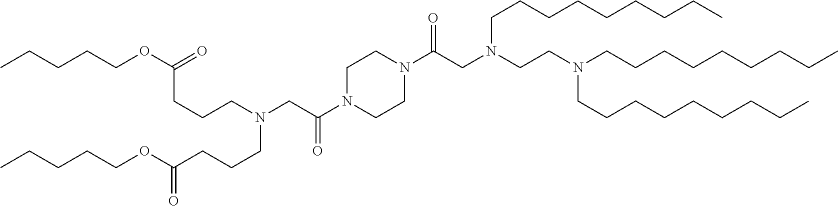 [SMILES: CCCCCCCCCN(CCCCCCCCC)CCN(CCCCCCCCC)CC(=O)N1CCN(C(=O)CN(CCCC(=O)OCCCCC)CCCC(=O)OCCCCC)CC1] |
| VI-73 | 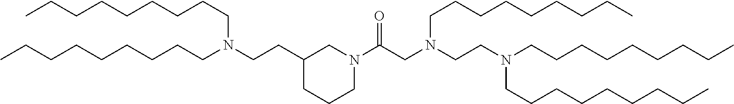 [SMILES: CCCCCCCCCN(CCCCCCCCC)CCC1CCCN(C(=O)CN(CCCCCCCCC)CCN(CCCCCCCCC)CCCCCCCCC)C1] |
| VI-74 | 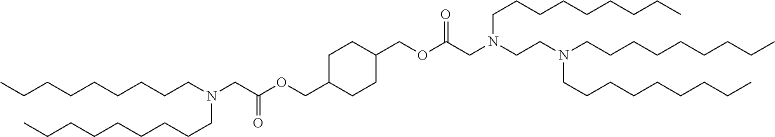 [SMILES: CCCCCCCCCN(CCCCCCCCC)CCN(CCCCCCCCC)CC(=O)OCC1CCC(COC(=O)CN(CCCCCCCCC)CCCCCCCCC)CC1] |
| VI-75 | 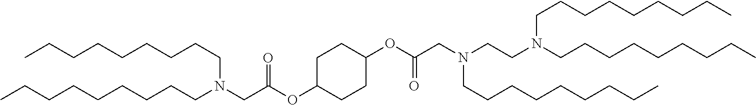 [SMILES: CCCCCCCCCN(CCCCCCCCC)CCN(CCCCCCCCC)CC(=O)OC1CCC(OC(=O)CN(CCCCCCCCC)CCCCCCCCC)CC1] |
| VI-76 | 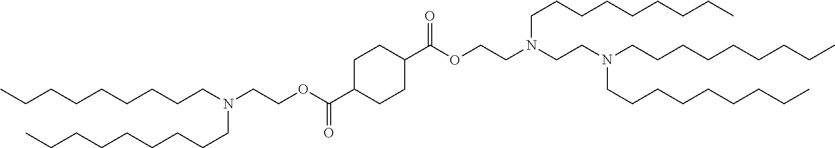 [SMILES: CCCCCCCCCN(CCCCCCCCC)CCOC(=O)C1CCC(C(=O)OCCN(CCCCCCCCC)CCN(CCCCCCCCC)CCCCCCCCC)CC1] |
| VI-77 | 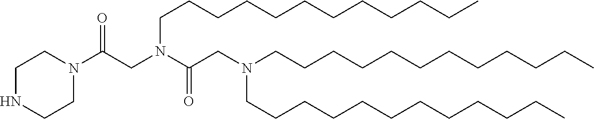 [SMILES: CCCCCCCCCCCCN(CCCCCCCCCCCC)CC(=O)N(CCCCCCCCCCCC)CC(=O)N1CCNCC1] |
| VI-78 | 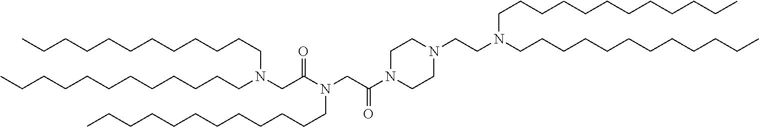 [SMILES: CCCCCCCCCCCCN(CCCCCCCCCCCC)CCN1CCN(C(=O)CN(CCCCCCCCCCCC)C(=O)CN(CCCCCCCCCCCC)CCCCCCCCCCCC)CC1] |
| VI-79 | 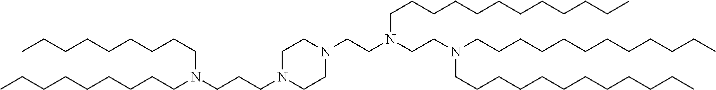 [SMILES: CCCCCCCCCCCCN(CCCCCCCCCCCC)CCN(CCCCCCCCCCCC)CCN1CCN(CCCN(CCCCCCCCC)CCCCCCCCC)CC1] |
| VI-80 | 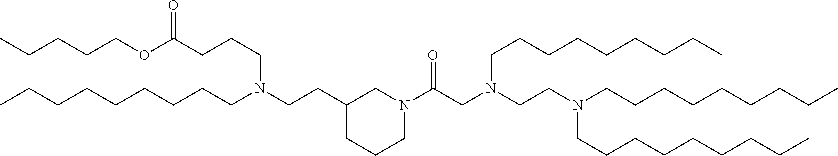 [SMILES: CCCCCCCCCN(CCCC(=O)OCCCCC)CCC1CCCN(C(=O)CN(CCCCCCCCC)CCN(CCCCCCCCC)CCCCCCCCC)C1] |
| VI-81 | 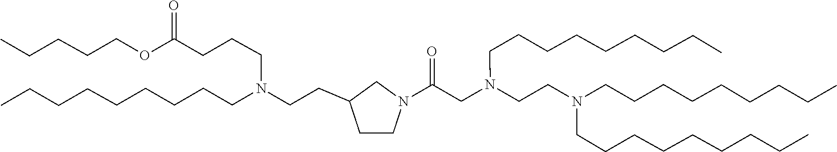 [SMILES: CCCCCCCCCN(CCCC(=O)OCCCCC)CCC1CCN(C(=O)CN(CCCCCCCCC)CCN(CCCCCCCCC)CCCCCCCCC)C1] |
| VI-82 | 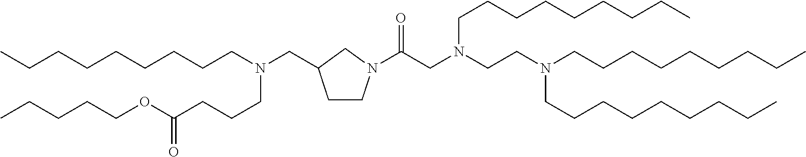 [SMILES: CCCCCCCCCN(CCCCCCCCC)CCN(CCCCCCCCC)CC(=O)N1CCC(CN(CCCCCCCCC)CCCC(=O)OCCCCC)C1] |
| VI-83 | 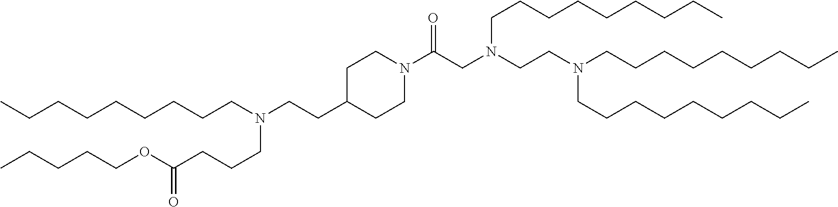 [SMILES: CCCCCCCCCN(CCCC(=O)OCCCCC)CCC1CCN(C(=O)CN(CCCCCCCCC)CCN(CCCCCCCCC)CCCCCCCCC)CC1] |
| VI-84 | 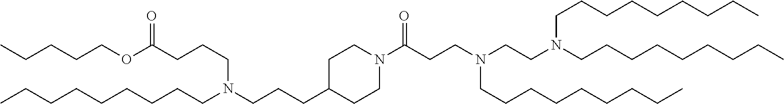 [SMILES: CCCCCCCCCN(CCCC(=O)OCCCCC)CCCC1CCN(C(=O)CCN(CCCCCCCCC)CCN(CCCCCCCCC)CCCCCCCCC)CC1] |
| VI-85 | 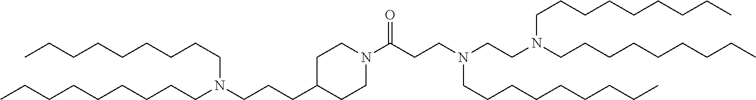 [SMILES: CCCCCCCCCN(CCCCCCCCC)CCCC1CCN(C(=O)CCN(CCCCCCCCC)CCN(CCCCCCCCC)CCCCCCCCC)CC1] |
| VI-86 | 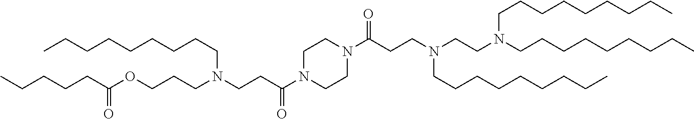 [SMILES: CCCCCCCCCN(CCCOC(=O)CCCCC)CCC(=O)N1CCN(C(=O)CCN(CCCCCCCCC)CCN(CCCCCCCCC)CCCCCCCCC)CC1] |
| VI-87 | 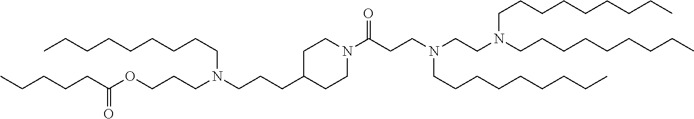 [SMILES: CCCCCCCCCN(CCCOC(=O)CCCCC)CCCC1CCN(C(=O)CCN(CCCCCCCCC)CCN(CCCCCCCCC)CCCCCCCCC)CC1] |
| VI-88 | 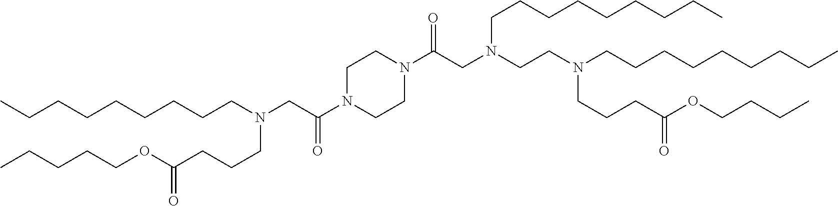 [SMILES: CCCCCCCCCN(CCCC(=O)OCCCC)CCN(CCCCCCCCC)CC(=O)N1CCN(C(=O)CN(CCCCCCCCC)CCCC(=O)OCCCCC)CC1] |
| VI-89 | 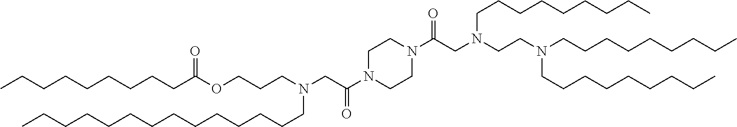 [SMILES: CCCCCCCCCCCCCCN(CCCOC(=O)CCCCCCCCC)CC(=O)N1CCN(C(=O)CN(CCCCCCCCC)CCN(CCCCCCCCC)CCCCCCCCC)CC1] |
| VI-90 | 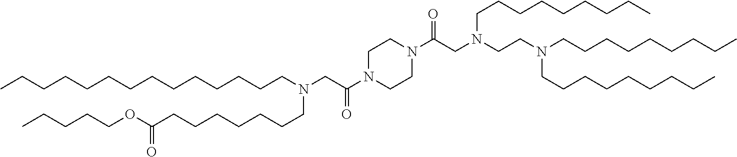 [SMILES: CCCCCCCCCCCCCCN(CCCCCCCC(=O)OCCCCC)CC(=O)N1CCN(C(=O)CN(CCCCCCCCC)CCN(CCCCCCCCC)CCCCCCCCC)CC1] |
| VI-91 | 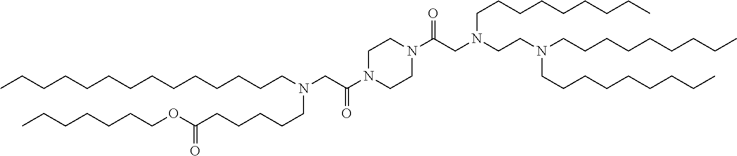 [SMILES: CCCCCCCCCCCCCCN(CCCCCC(=O)OCCCCCCC)CC(=O)N1CCN(C(=O)CN(CCCCCCCCC)CCN(CCCCCCCCC)CCCCCCCCC)CC1] |
| VI-92 | 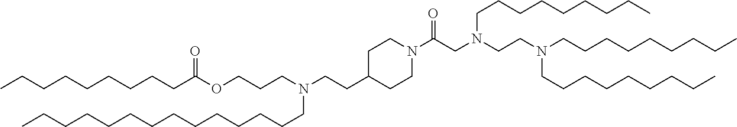 [SMILES: CCCCCCCCCCCCCCN(CCCOC(=O)CCCCCCCCC)CCC1CCN(C(=O)CN(CCCCCCCCC)CCN(CCCCCCCCC)CCCCCCCCC)CC1] |
| VI-93 | 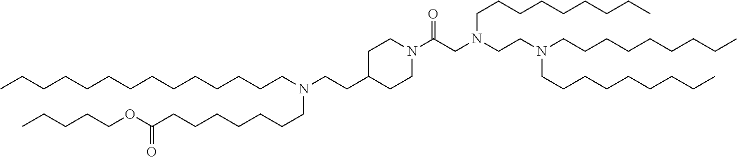 [SMILES: CCCCCCCCCCCCCCN(CCCCCCCC(=O)OCCCCC)CCC1CCN(C(=O)CN(CCCCCCCCC)CCN(CCCCCCCCC)CCCCCCCCC)CC1] |
| VI-94 | 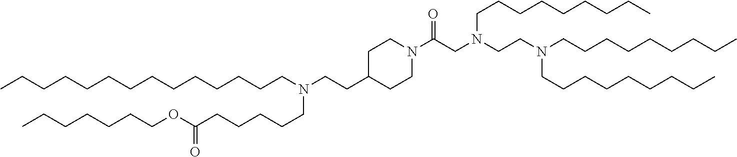 |
| VI-95 | 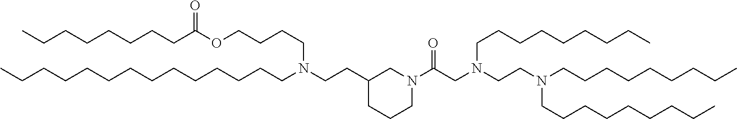 [SMILES: CCCCCCCCCCCCCCN(CCCCOC(=O)CCCCCCCC)CCC1CCCN(C(=O)CN(CCCCCCCCC)CCN(CCCCCCCCC)CCCCCCCCC)C1] |
| VI-96 | 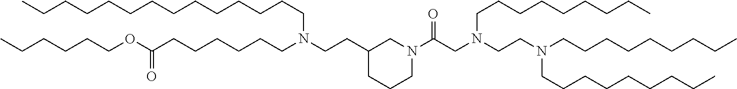 [SMILES: CCCCCCCCCCCCCCN(CCCCCCC(=O)OCCCCCC)CCC1CCCN(C(=O)CN(CCCCCCCCC)CCN(CCCCCCCCC)CCCCCCCCC)C1] |
| VI-97 | 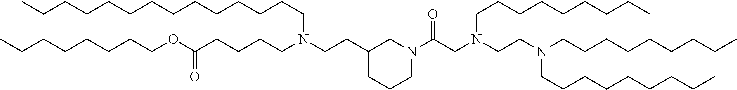 [SMILES: CCCCCCCCCCCCCCN(CCCCC(=O)OCCCCCCCC)CCC1CCCN(C(=O)CN(CCCCCCCCC)CCN(CCCCCCCCC)CCCCCCCCC)C1] |
| VI-98 | 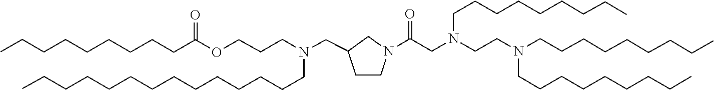 [SMILES: CCCCCCCCCCCCCCN(CCCOC(=O)CCCCCCCCC)CC1CCN(C(=O)CN(CCCCCCCCC)CCN(CCCCCCCCC)CCCCCCCCC)C1] |
| VI-99 | 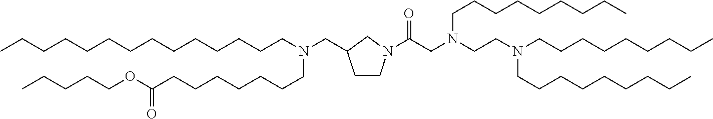 [SMILES: CCCCCCCCCCCCCCN(CCCCCCCC(=O)OCCCCC)CC1CCN(C(=O)CN(CCCCCCCCC)CCN(CCCCCCCCC)CCCCCCCCC)C1] |
| VI- 100 | 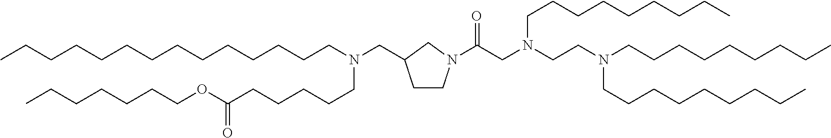 [SMILES: CCCCCCCCCCCCCCN(CCCCCC(=O)OCCCCCCC)CC1CCN(C(=O)CN(CCCCCCCCC)CCN(CCCCCCCCC)CCCCCCCCC)C1] |
| VI- 101 | 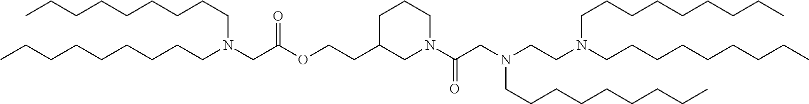 [SMILES: CCCCCCCCCN(CCCCCCCCC)CCN(CCCCCCCCC)CC(=O)N1CCCC(CCOC(=O)CN(CCCCCCCCC)CCCCCCCCC)C1] |
| VI- 102 | 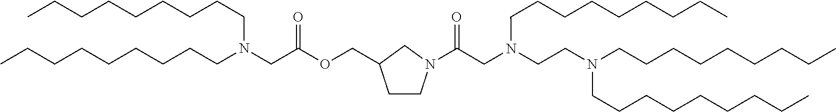 [SMILES: CCCCCCCCCN(CCCCCCCCC)CCN(CCCCCCCCC)CC(=O)N1CCC(COC(=O)CN(CCCCCCCCC)CCCCCCCCC)C1] |
| VI- 103 | 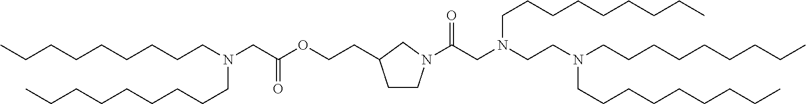 [SMILES: CCCCCCCCCN(CCCCCCCCC)CCN(CCCCCCCCC)CC(=O)N1CCC(CCOC(=O)CN(CCCCCCCCC)CCCCCCCCC)C1] |
| VI- 104 | 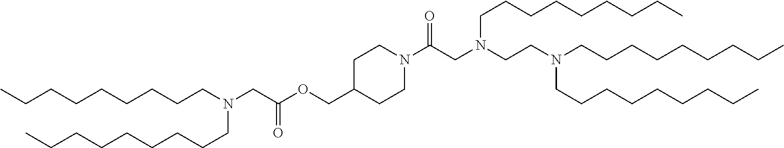 [SMILES: CCCCCCCCCN(CCCCCCCCC)CCN(CCCCCCCCC)CC(=O)N1CCC(COC(=O)CN(CCCCCCCCC)CCCCCCCCC)CC1] |
| VI- 105 | 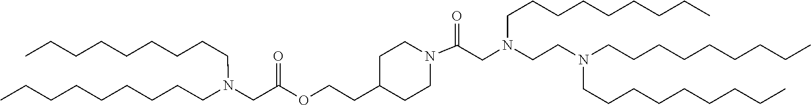 [SMILES: CCCCCCCCCN(CCCCCCCCC)CCN(CCCCCCCCC)CC(=O)N1CCC(CCOC(=O)CN(CCCCCCCCC)CCCCCCCCC)CC1] |
| VI- 106 | 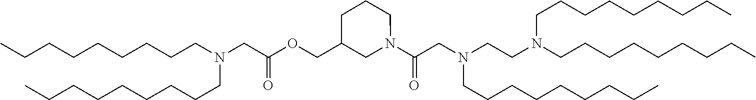 [SMILES: CCCCCCCCCN(CCCCCCCCC)CCN(CCCCCCCCC)CC(=O)N1CCCC(COC(=O)CN(CCCCCCCCC)CCCCCCCCC)C1] |
| VI- 107 | 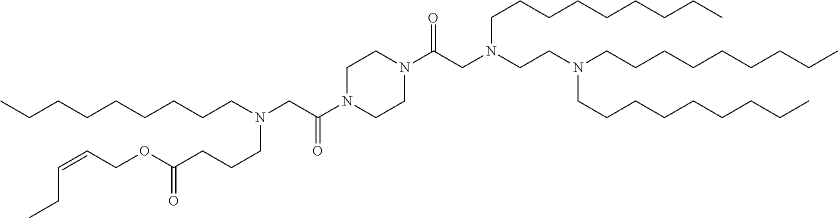 [SMILES: CC/C=C\COC(=O)CCCN(CCCCCCCCC)CC(=O)N1CCN(C(=O)CN(CCCCCCCCC)CCN(CCCCCCCCC)CCCCCCCCC)CC1] |
| VI- 108 | 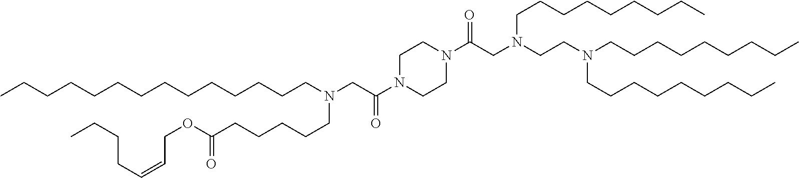 [SMILES: CCCC/C=C\COC(=O)CCCCCN(CCCCCCCCCCCCCC)CC(=O)N1CCN(C(=O)CN(CCCCCCCCC)CCN(CCCCCCCCC)CCCCCCCCC)CC1] |
| VI- 109 | 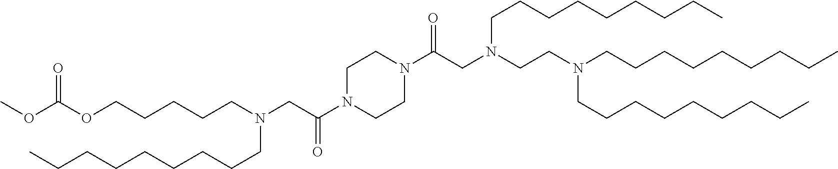 [SMILES: CCCCCCCCCN(CCCCCCCCC)CCN(CCCCCCCCC)CC(=O)N1CCN(C(=O)CN(CCCCCCCCC)CCCCCOC(=O)OC)CC1] |
| VI- 110 | 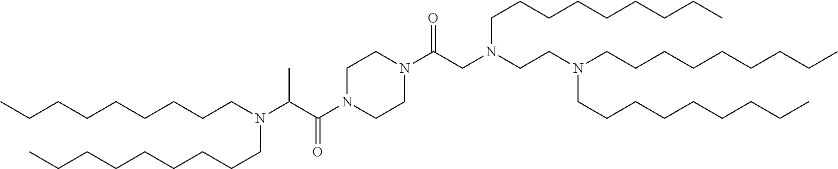 [SMILES: CCCCCCCCCN(CCCCCCCCC)CCN(CCCCCCCCC)CC(=O)N1CCN(C(=O)C(C)N(CCCCCCCCC)CCCCCCCCC)CC1] |
| VI- 111 | 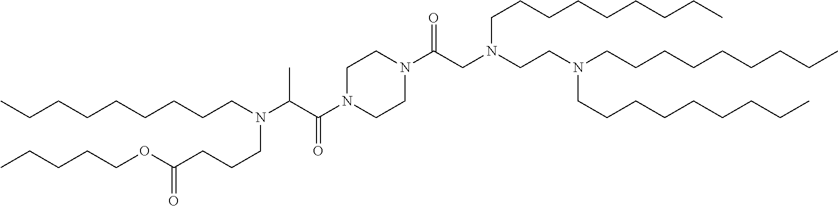 [SMILES: CCCCCCCCCN(CCCCCCCCC)CCN(CCCCCCCCC)CC(=O)N1CCN(C(=O)C(C)N(CCCCCCCCC)CCCC(=O)OCCCCC)CC1] |
| VI- 112 | 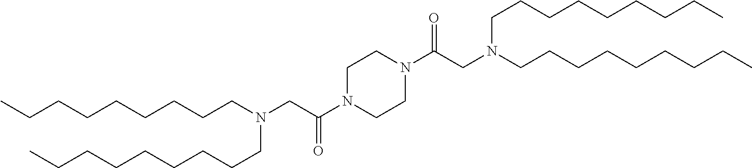 [SMILES: CCCCCCCCCN(CCCCCCCCC)CC(=O)N1CCN(C(=O)CN(CCCCCCCCC)CCCCCCCCC)CC1] |
| VI- 113 | 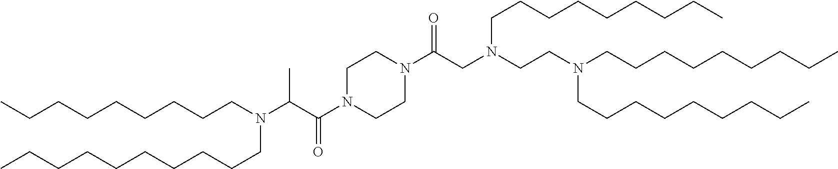 [SMILES: CCCCCCCCCCN(CCCCCCCCC)C(C)C(=O)N1CCN(C(=O)CN(CCCCCCCCC)CCN(CCCCCCCCC)CCCCCCCCC)CC1] |
| VI- 114 | 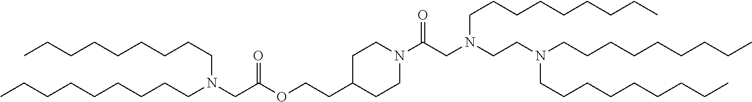 [SMILES: CCCCCCCCCN(CCCCCCCCC)CCN(CCCCCCCCC)CC(=O)N1CCC(CCOC(=O)CN(CCCCCCCCC)CCCCCCCCC)CC1] |
| VI- 115 | 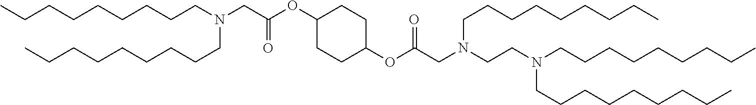 [SMILES: CCCCCCCCCN(CCCCCCCCC)CCN(CCCCCCCCC)CC(=O)OC1CCC(OC(=O)CN(CCCCCCCCC)CCCCCCCCC)CC1] |
| VI- 116 | 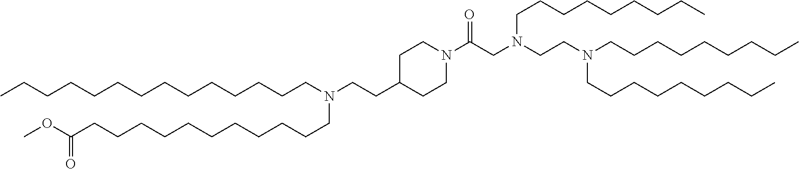 [SMILES: CCCCCCCCCCCCCCN(CCCCCCCCCCCC(=O)OC)CCC1CCN(C(=O)CN(CCCCCCCCC)CCN(CCCCCCCCC)CCCCCCCCC)CC1] |
| VI- 117 | 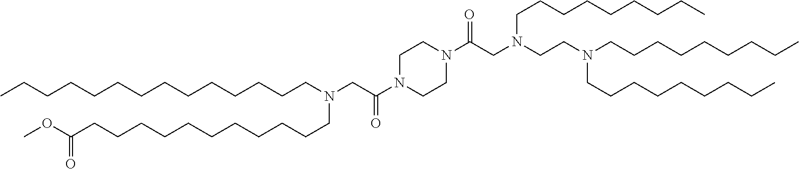 [SMILES: CCCCCCCCCCCCCCN(CCCCCCCCCCCC(=O)OC)CC(=O)N1CCN(C(=O)CN(CCCCCCCCC)CCN(CCCCCCCCC)CCCCCCCCC)CC1] |
| VI- 118 | 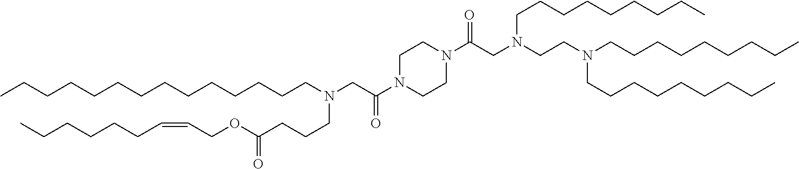 [SMILES: CCCCCC/C=C\COC(=O)CCCN(CCCCCCCCCCCCCC)CC(=O)N1CCN(C(=O)CN(CCCCCCCCC)CCN(CCCCCCCCC)CCCCCCCCC)CC1] |
| VI- 119 | 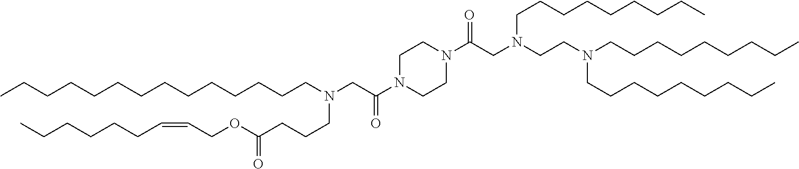 [SMILES: CCCCCC/C=C\COC(=O)CCCN(CCCCCCCCCCCCCC)CC(=O)N1CCN(C(=O)CN(CCCCCCCCC)CCN(CCCCCCCCC)CCCCCCCCC)CC1] |
| VI- 120 | 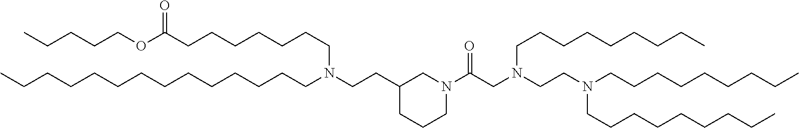 [SMILES: CCCCCCCCCCCCCCN(CCCCCCCC(=O)OCCCCC)CCC1CCCN(C(=O)CN(CCCCCCCCC)CCN(CCCCCCCCC)CCCCCCCCC)C1] |
| VI- 121 | 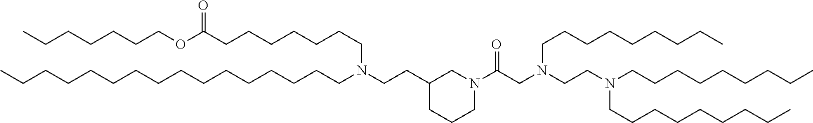 [SMILES: CCCCCCCCCCCCCCCCN(CCCCCCCC(=O)OCCCCCCC)CCC1CCCN(C(=O)CN(CCCCCCCCC)CCN(CCCCCCCCC)CCCCCCCCC)C1] |
| VI- 122 | 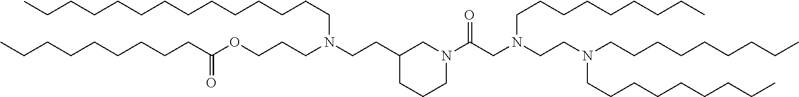 [SMILES: CCCCCCCCCCCCCCN(CCCOC(=O)CCCCCCCCC)CCC1CCCN(C(=O)CN(CCCCCCCCC)CCN(CCCCCCCCC)CCCCCCCCC)C1] |
| VI- 123 | 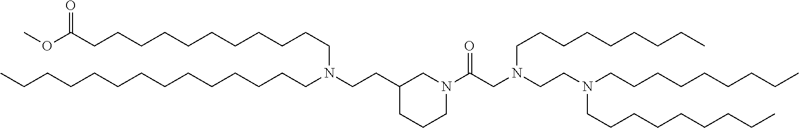 [SMILES: CCCCCCCCCCCCCCN(CCCCCCCCCCCC(=O)OC)CCC1CCCN(C(=O)CN(CCCCCCCCC)CCN(CCCCCCCCC)CCCCCCCCC)C1] |
| VI- 124 | 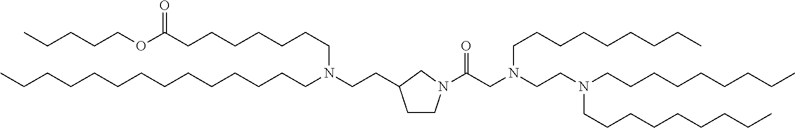 [SMILES: CCCCCCCCCCCCCCN(CCCCCCCC(=O)OCCCCC)CCC1CCN(C(=O)CN(CCCCCCCCC)CCN(CCCCCCCCC)CCCCCCCCC)C1] |
| VI- 125 | 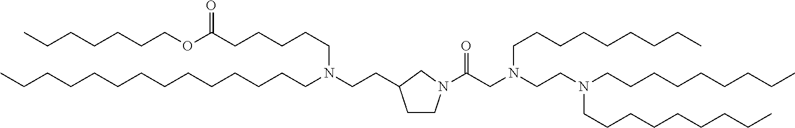 [SMILES: CCCCCCCCCCCCCCN(CCCCCC(=O)OCCCCCCC)CCC1CCN(C(=O)CN(CCCCCCCCC)CCN(CCCCCCCCC)CCCCCCCCC)C1] |
| VI- 126 | 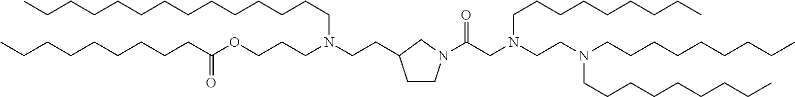 [SMILES: CCCCCCCCCCCCCCN(CCCOC(=O)CCCCCCCCC)CCC1CCN(C(=O)CN(CCCCCCCCC)CCN(CCCCCCCCC)CCCCCCCCC)C1] |
| VI- 127 | 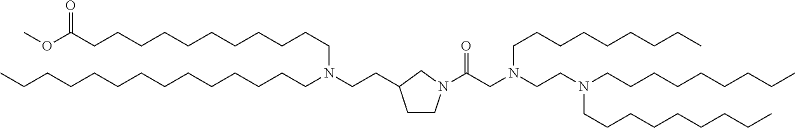 [SMILES: CCCCCCCCCCCCCCN(CCCCCCCCCCCC(=O)OC)CCC1CCN(C(=O)CN(CCCCCCCCC)CCN(CCCCCCCCC)CCCCCCCCC)C1] |
| VI- 128 | 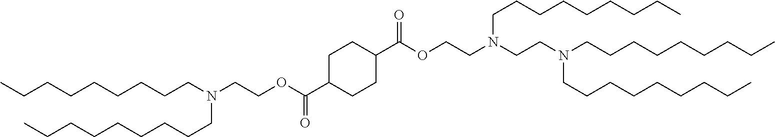 [SMILES: CCCCCCCCCN(CCCCCCCCC)CCOC(=O)C1CCC(C(=O)OCCN(CCCCCCCCC)CCN(CCCCCCCCC)CCCCCCCCC)CC1] |
| VI- 129 | 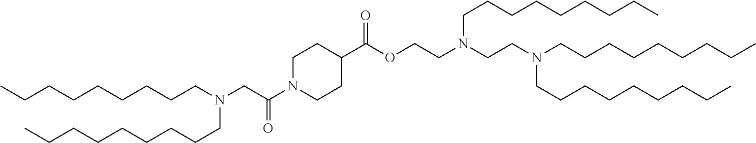 [SMILES: CCCCCCCCCN(CCCCCCCCC)CCN(CCCCCCCCC)CCOC(=O)C1CCN(C(=O)CN(CCCCCCCCC)CCCCCCCCC)CC1] |
| VI- 130 | 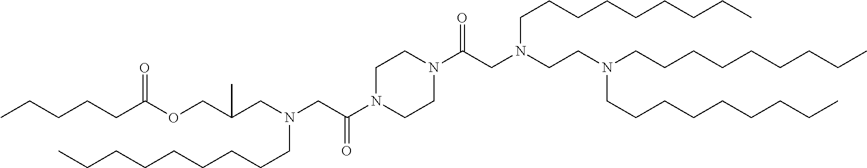 [SMILES: CCCCCCCCCN(CCCCCCCCC)CCN(CCCCCCCCC)CC(=O)N1CCN(C(=O)CN(CCCCCCCCC)CC(C)COC(=O)CCCCC)CC1] |
| VI- 131 | 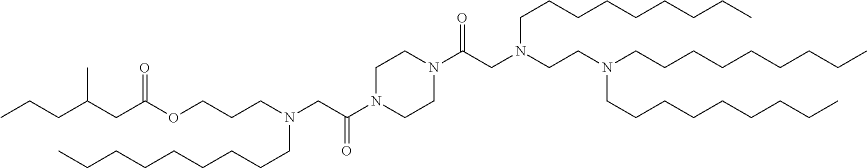 [SMILES: CCCCCCCCCN(CCCCCCCCC)CCN(CCCCCCCCC)CC(=O)N1CCN(C(=O)CN(CCCCCCCCC)CCCOC(=O)CC(C)CCC)CC1] |
| VI- 132 | 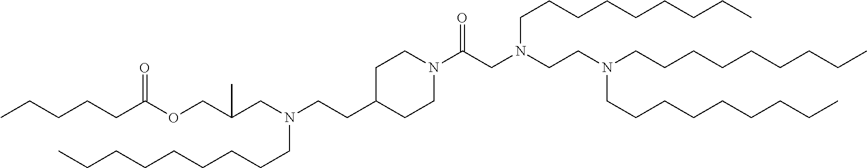 [SMILES: CCCCCCCCCN(CCCCCCCCC)CCN(CCCCCCCCC)CC(=O)N1CCC(CCN(CCCCCCCCC)CC(C)COC(=O)CCCCC)CC1] |
| VI- 133 | 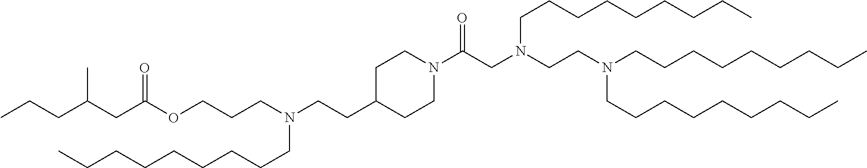 [SMILES: CCCCCCCCCN(CCCOC(=O)CC(C)CCC)CCC1CCN(C(=O)CN(CCCCCCCCC)CCN(CCCCCCCCC)CCCCCCCCC)CC1] |
| VI- 134 | 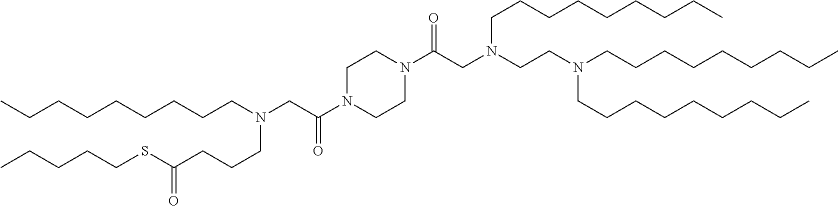 [SMILES: CCCCCCCCCN(CCCCCCCCC)CCN(CCCCCCCCC)CC(=O)N1CCN(C(=O)CN(CCCCCCCCC)CCCC(=O)SCCCCC)CC1] |
| VI- 135 | 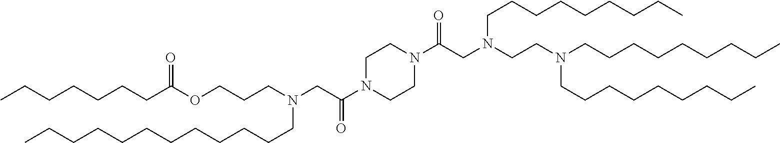 [SMILES: CCCCCCCCCCCCN(CCCOC(=O)CCCCCCC)CC(=O)N1CCN(C(=O)CN(CCCCCCCCC)CCN(CCCCCCCCC)CCCCCCCCC)CC1] |
| VI- 136 | 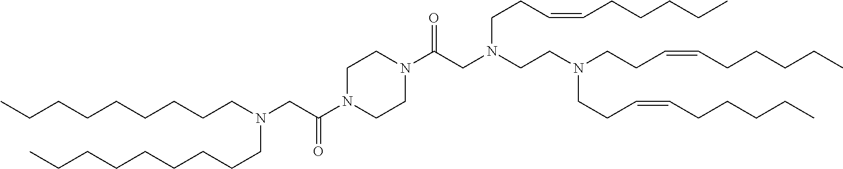 [SMILES: CCCCC/C=C\CCN(CC/C=C\CCCCC)CCN(CC/C=C\CCCCC)CC(=O)N1CCN(C(=O)CN(CCCCCCCCC)CCCCCCCCC)CC1] |
| VI- 137 | 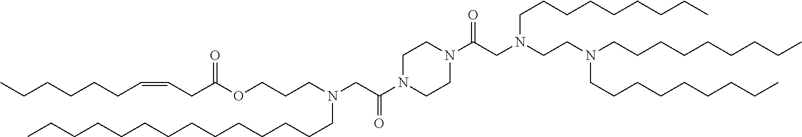 [SMILES: CCCCCC/C=C\CC(=O)OCCCN(CCCCCCCCCCCCCC)CC(=O)N1CCN(C(=O)CN(CCCCCCCCC)CCN(CCCCCCCCC)CCCCCCCCC)CC1] |
| VI- 138 | 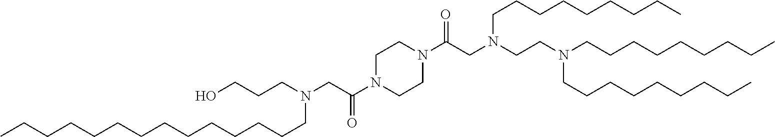 [SMILES: CCCCCCCCCCCCCCN(CCCO)CC(=O)N1CCN(C(=O)CN(CCCCCCCCC)CCN(CCCCCCCCC)CCCCCCCCC)CC1] |
| VI- 139 | 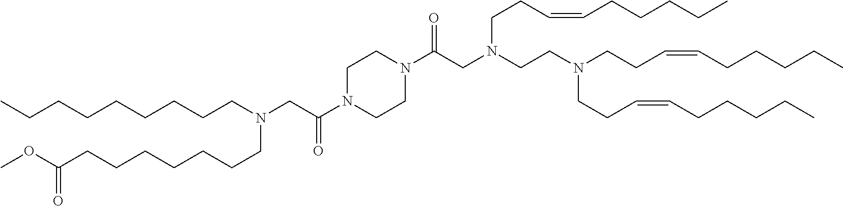 [SMILES: CCCCC/C=C\CCN(CC/C=C\CCCCC)CCN(CC/C=C\CCCCC)CC(=O)N1CCN(C(=O)CN(CCCCCCCCC)CCCCCCCC(=O)OC)CC1] |
| VI- 140 | 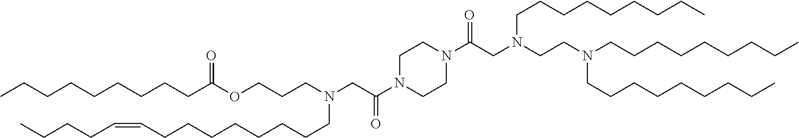 [SMILES: CCCC/C=C\CCCCCCCCN(CCCOC(=O)CCCCCCCCC)CC(=O)N1CCN(C(=O)CN(CCCCCCCCC)CCN(CCCCCCCCC)CCCCCCCCC)CC1] |
| VI- 141 | 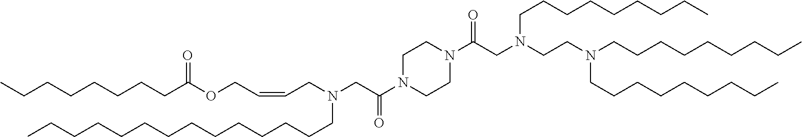 [SMILES: CCCCCCCCCCCCCCN(C/C=C\COC(=O)CCCCCCCC)CC(=O)N1CCN(C(=O)CN(CCCCCCCCC)CCN(CCCCCCCCC)CCCCCCCCC)CC1] |
| VI- 142 | 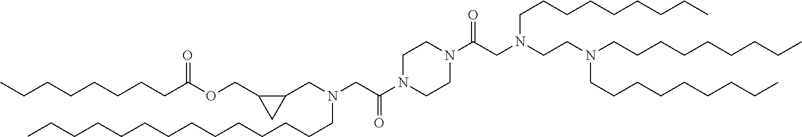 [SMILES: CCCCCCCCCCCCCCN(CC(=O)N1CCN(C(=O)CN(CCCCCCCCC)CCN(CCCCCCCCC)CCCCCCCCC)CC1)CC1CC1COC(=O)CCCCCCCC] |
| VI- 143 | 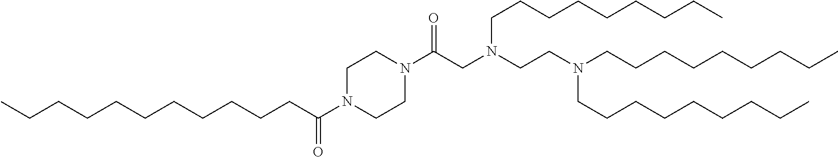 [SMILES: CCCCCCCCCCCC(=O)N1CCN(C(=O)CN(CCCCCCCCC)CCN(CCCCCCCCC)CCCCCCCCC)CC1] |
| VI- 144 | 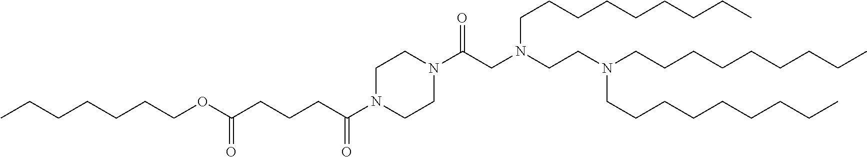 [SMILES: CCCCCCCCCN(CCCCCCCCC)CCN(CCCCCCCCC)CC(=O)N1CCN(C(=O)CCCC(=O)OCCCCCCC)CC1] |
| VI- 145 | 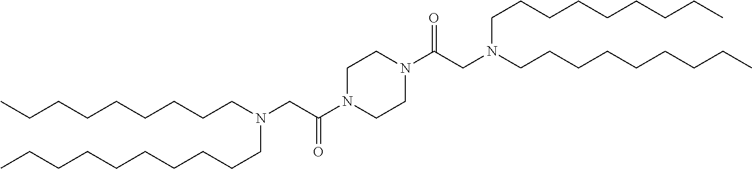 [SMILES: CCCCCCCCCCN(CCCCCCCCC)CC(=O)N1CCN(C(=O)CN(CCCCCCCCC)CCCCCCCCC)CC1] |
| VI- 146 | 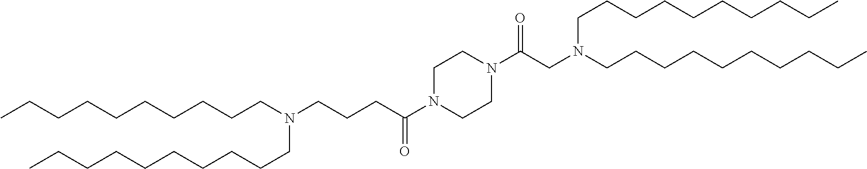 [SMILES: CCCCCCCCCCN(CCCCCCCCCC)CCCC(=O)N1CCN(C(=O)CN(CCCCCCCCCC)CCCCCCCCCC)CC1] |
| VI- 147 | 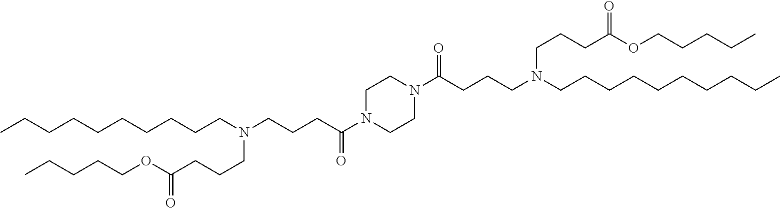 [SMILES: CCCCCCCCCCN(CCCC(=O)OCCCCC)CCCC(=O)N1CCN(C(=O)CCCN(CCCCCCCCCC)CCCC(=O)OCCCCC)CC1] |
| VI- 148 | 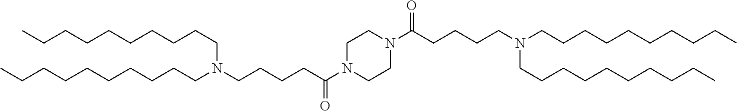 [SMILES: CCCCCCCCCCN(CCCCCCCCCC)CCCCC(=O)N1CCN(C(=O)CCCCN(CCCCCCCCCC)CCCCCCCCCC)CC1] |
| VI- 149 | 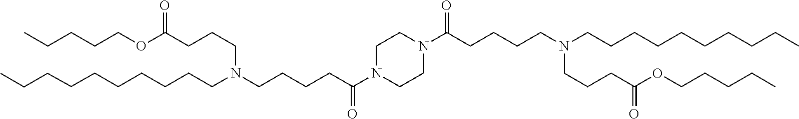 |
| VI- 150 | 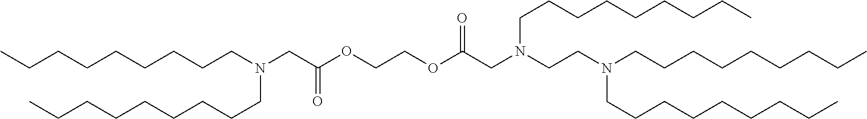 [SMILES: CCCCCCCCCN(CCCCCCCCC)CCN(CCCCCCCCC)CC(=O)OCCOC(=O)CN(CCCCCCCCC)CCCCCCCCC] |
| VI- 151 | 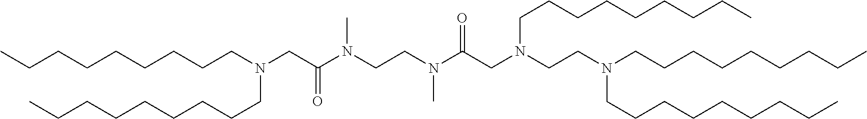 [SMILES: CCCCCCCCCN(CCCCCCCCC)CCN(CCCCCCCCC)CC(=O)N(C)CCN(C)C(=O)CN(CCCCCCCCC)CCCCCCCCC] |
| VI- 152 | 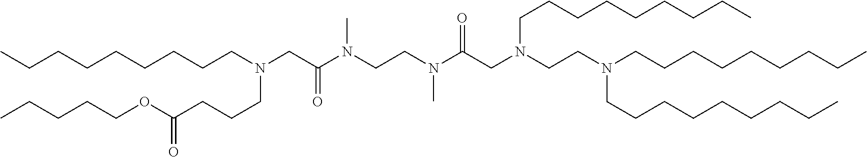 [SMILES: CCCCCCCCCN(CCCCCCCCC)CCN(CCCCCCCCC)CC(=O)N(C)CCN(C)C(=O)CN(CCCCCCCCC)CCCC(=O)OCCCCC] |
| VI- 153 | 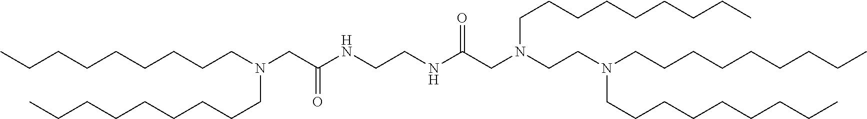 [SMILES: CCCCCCCCCN(CCCCCCCCC)CCN(CCCCCCCCC)CC(=O)NCCNC(=O)CN(CCCCCCCCC)CCCCCCCCC] |
| VI- 154 | 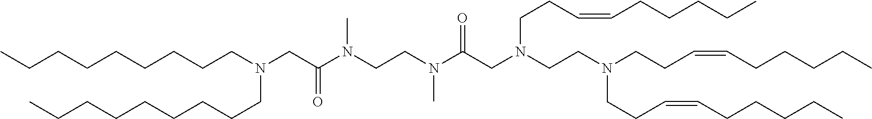 [SMILES: CCCCC/C=C\CCN(CC/C=C\CCCCC)CCN(CC/C=C\CCCCC)CC(=O)N(C)CCN(C)C(=O)CN(CCCCCCCCC)CCCCCCCCC] |
| VI- 155 | 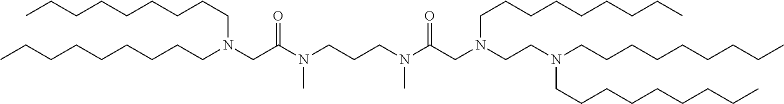 [SMILES: CCCCCCCCCN(CCCCCCCCC)CCN(CCCCCCCCC)CC(=O)N(C)CCCN(C)C(=O)CN(CCCCCCCCC)CCCCCCCCC] |
| VI- 156 | 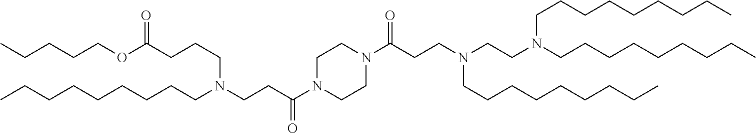 [SMILES: CCCCCCCCCN(CCCC(=O)OCCCCC)CCC(=O)N1CCN(C(=O)CCN(CCCCCCCCC)CCN(CCCCCCCCC)CCCCCCCCC)CC1] |
| VI- 157 | 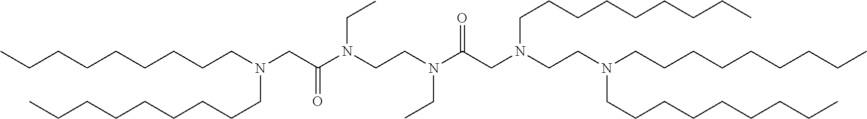 [SMILES: CCCCCCCCCN(CCCCCCCCC)CCN(CCCCCCCCC)CC(=O)N(CC)CCN(CC)C(=O)CN(CCCCCCCCC)CCCCCCCCC] |
| VI- 158 | 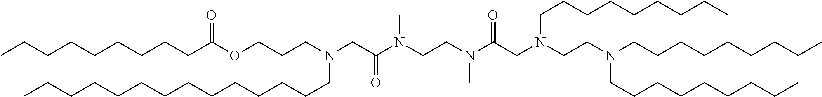 [SMILES: CCCCCCCCCCCCCCN(CCCOC(=O)CCCCCCCCC)CC(=O)N(C)CCN(C)C(=O)CN(CCCCCCCCC)CCN(CCCCCCCCC)CCCCCCCCC] |
| VI- 159 | 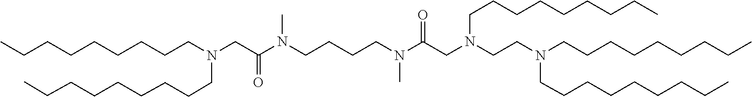 [SMILES: CCCCCCCCCN(CCCCCCCCC)CCN(CCCCCCCCC)CC(=O)N(C)CCCCN(C)C(=O)CN(CCCCCCCCC)CCCCCCCCC] |
| VI- 160 | 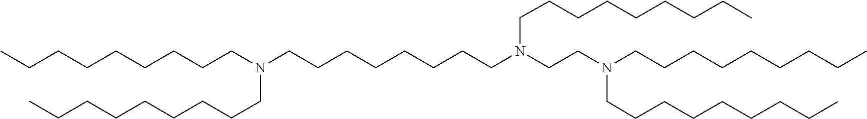 [SMILES: CCCCCCCCCN(CCCCCCCCC)CCCCCCCCN(CCCCCCCCC)CCN(CCCCCCCCC)CCCCCCCCC] |
| VI- 161 | 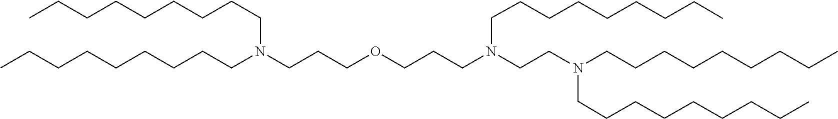 [SMILES: CCCCCCCCCN(CCCCCCCCC)CCCOCCCN(CCCCCCCCC)CCN(CCCCCCCCC)CCCCCCCCC] |
| VI- 162 | 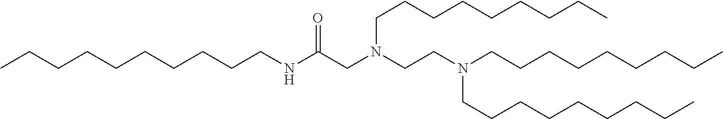 [SMILES: CCCCCCCCCCNC(=O)CN(CCCCCCCCC)CCN(CCCCCCCCC)CCCCCCCCC] |
| VI- 163 | 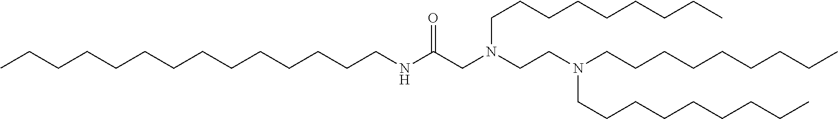 [SMILES: CCCCCCCCCCCCCCNC(=O)CN(CCCCCCCCC)CCN(CCCCCCCCC)CCCCCCCCC] |
| VI- 164 | 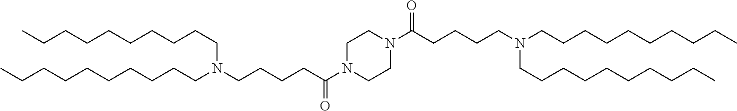 [SMILES: CCCCCCCCCCN(CCCCCCCCCC)CCCCC(=O)N1CCN(C(=O)CCCCN(CCCCCCCCCC)CCCCCCCCCC)CC1] |
| VI- 165 | 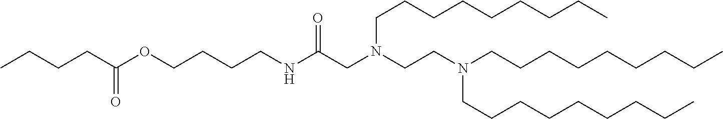 [SMILES: CCCCCCCCCN(CCCCCCCCC)CCN(CCCCCCCCC)CC(=O)NCCCCOC(=O)CCCC] |
| VI- 166 | 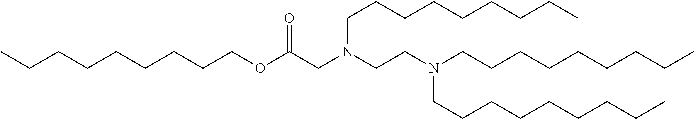 [SMILES: CCCCCCCCCOC(=O)CN(CCCCCCCCC)CCN(CCCCCCCCC)CCCCCCCCC] |
| VI- 167 | 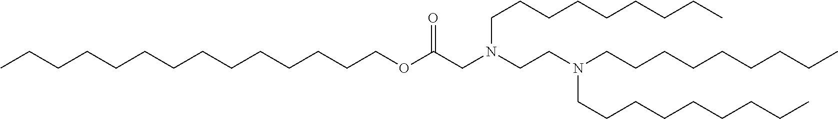 [SMILES: CCCCCCCCCCCCCCOC(=O)CN(CCCCCCCCC)CCN(CCCCCCCCC)CCCCCCCCC] |
| VI- 168 | 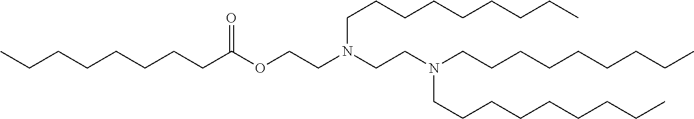 [SMILES: CCCCCCCCCN(CCCCCCCCC)CCN(CCCCCCCCC)CCOC(=O)CCCCCCCC] |
| VI- 169 | 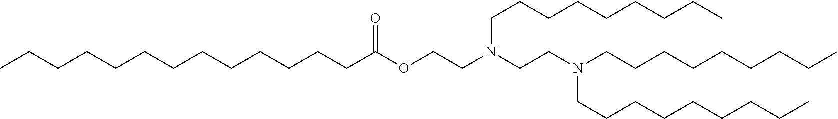 [SMILES: CCCCCCCCCCCCCC(=O)OCCN(CCCCCCCCC)CCN(CCCCCCCCC)CCCCCCCCC] |
| VI- 170 | 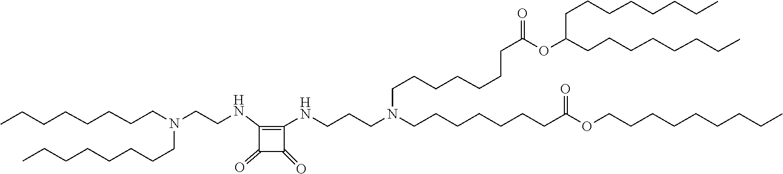 [SMILES: CCCCCCCCCOC(=O)CCCCCCCN(CCCCCCCC(=O)OC(CCCCCCCC)CCCCCCCC)CCCNc1c(NCCN(CCCCCCCC)CCCCCCCC)c(=O)c1=O] |
| VI- 171 | 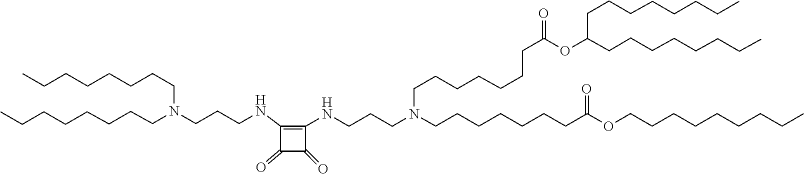 [SMILES: CCCCCCCCCOC(=O)CCCCCCCN(CCCCCCCC(=O)OC(CCCCCCCC)CCCCCCCC)CCCNc1c(NCCCN(CCCCCCCC)CCCCCCCC)c(=O)c1=O] |
| VI- 172 | 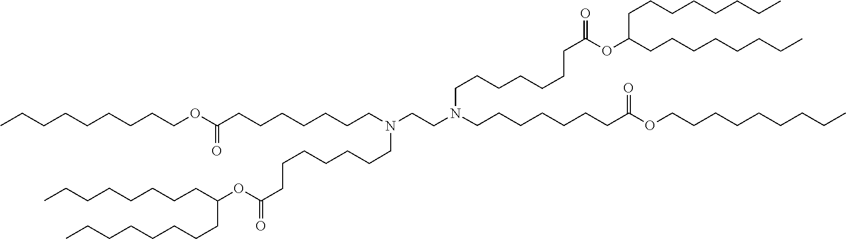 [SMILES: CCCCCCCCCOC(=O)CCCCCCCN(CCCCCCCC(=O)OC(CCCCCCCC)CCCCCCCC)CCN(CCCCCCCC(=O)OCCCCCCCCC)CCCCCCCC(=O)OC(CCCCCCCC)CCCCCCCC] |
| VI- 173 | 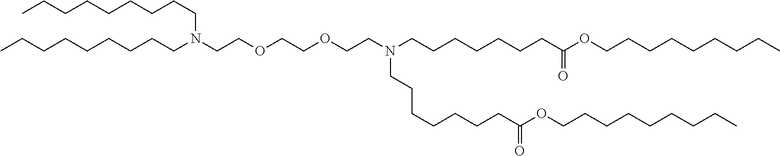 [SMILES: CCCCCCCCCOC(=O)CCCCCCCN(CCCCCCCC(=O)OCCCCCCCCC)CCOCCOCCN(CCCCCCCCC)CCCCCCCCC] |
| VI- 174 | 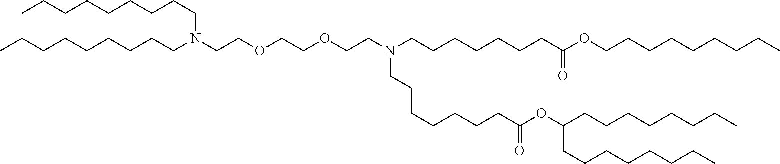 [SMILES: CCCCCCCCCOC(=O)CCCCCCCN(CCCCCCCC(=O)OC(CCCCCCCC)CCCCCCCC)CCOCCOCCN(CCCCCCCCC)CCCCCCCCC] |
| VI- 175 | 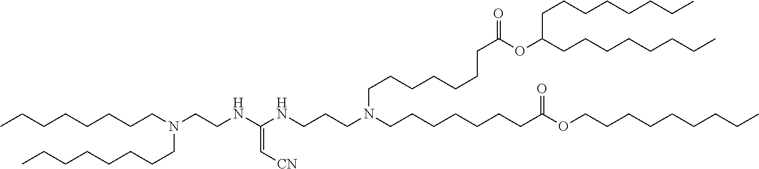 [SMILES: CCCCCCCCCOC(=O)CCCCCCCN(CCCCCCCC(=O)OC(CCCCCCCC)CCCCCCCC)CCCN/C(=C\C#N)NCCN(CCCCCCCC)CCCCCCCC] |
| VI- 176 | 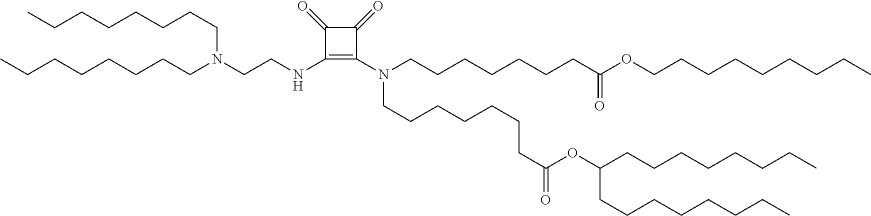 [SMILES: CCCCCCCCCOC(=O)CCCCCCCN(CCCCCCCC(=O)OC(CCCCCCCC)CCCCCCCC)c1c(NCCN(CCCCCCCC)CCCCCCCC)c(=O)c1=O] |
| VI- 177 | 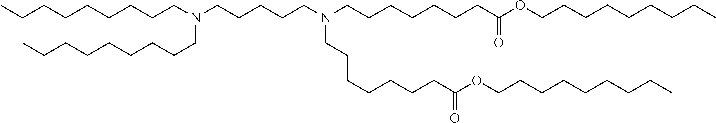 [SMILES: CCCCCCCCCOC(=O)CCCCCCCN(CCCCCCCC(=O)OCCCCCCCCC)CCCCCN(CCCCCCCCC)CCCCCCCCC] |
| VI- 178 | 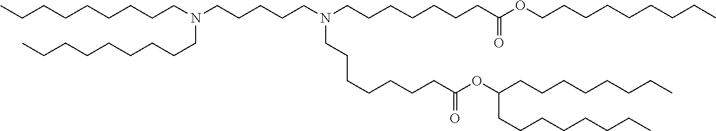 [SMILES: CCCCCCCCCOC(=O)CCCCCCCN(CCCCCCCC(=O)OC(CCCCCCCC)CCCCCCCC)CCCCCN(CCCCCCCCC)CCCCCCCCC] |
| VI- 179 | 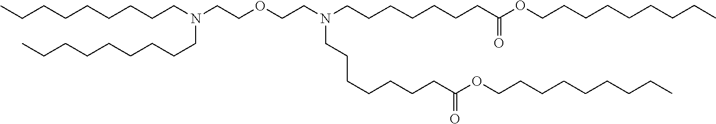 [SMILES: CCCCCCCCCOC(=O)CCCCCCCN(CCCCCCCC(=O)OCCCCCCCCC)CCOCCN(CCCCCCCCC)CCCCCCCCC] |
| VI- 180 | 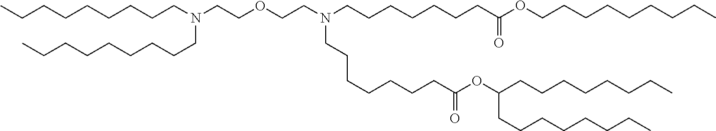 [SMILES: CCCCCCCCCOC(=O)CCCCCCCN(CCCCCCCC(=O)OC(CCCCCCCC)CCCCCCCC)CCOCCN(CCCCCCCCC)CCCCCCCCC] |
| VI- 181 | 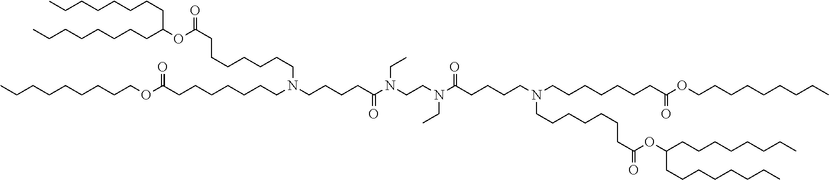 [SMILES: CCCCCCCCCOC(=O)CCCCCCCN(CCCCCCCC(=O)OC(CCCCCCCC)CCCCCCCC)CCCCC(=O)N(CC)CCN(CC)C(=O)CCCCN(CCCCCCCC(=O)OCCCCCCCCC)CCCCCCCC(=O)OC(CCCCCCCC)CCCCCCCC] |
| VI- 182 | 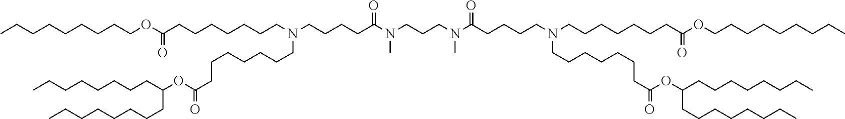 [SMILES: CCCCCCCCCOC(=O)CCCCCCCN(CCCCCCCC(=O)OC(CCCCCCCC)CCCCCCCC)CCCCC(=O)N(C)CCCN(C)C(=O)CCCCN(CCCCCCCC(=O)OCCCCCCCCC)CCCCCCCC(=O)OC(CCCCCCCC)CCCCCCCC] |
| VI- 183 | 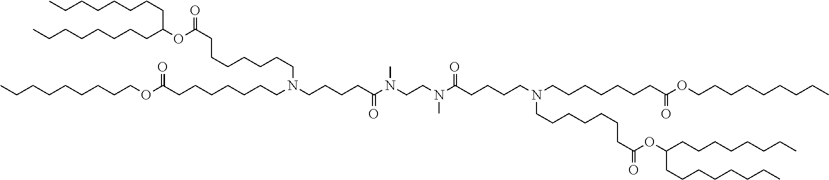 [SMILES: CCCCCCCCCOC(=O)CCCCCCCN(CCCCCCCC(=O)OC(CCCCCCCC)CCCCCCCC)CCCCC(=O)N(C)CCN(C)C(=O)CCCCN(CCCCCCCC(=O)OCCCCCCCCC)CCCCCCCC(=O)OC(CCCCCCCC)CCCCCCCC] |
| VI- 184 | 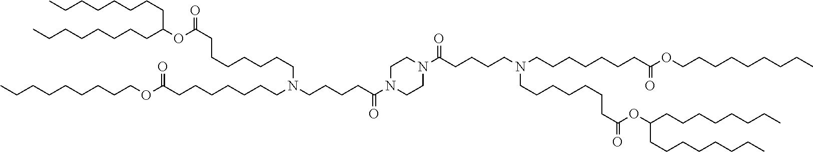 [SMILES: CCCCCCCCCOC(=O)CCCCCCCN(CCCCCCCC(=O)OC(CCCCCCCC)CCCCCCCC)CCCCC(=O)N1CCN(C(=O)CCCCN(CCCCCCCC(=O)OCCCCCCCCC)CCCCCCCC(=O)OC(CCCCCCCC)CCCCCCCC)CC1] |
| VI- 185 | 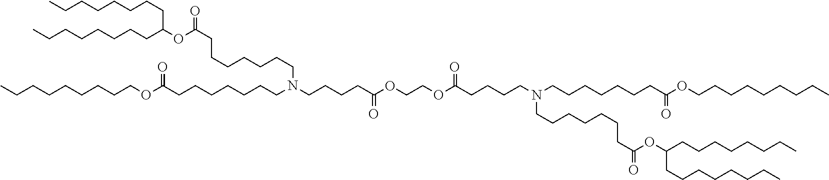 [SMILES: CCCCCCCCCOC(=O)CCCCCCCN(CCCCCCCC(=O)OC(CCCCCCCC)CCCCCCCC)CCCCC(=O)OCCOC(=O)CCCCN(CCCCCCCC(=O)OCCCCCCCCC)CCCCCCCC(=O)OC(CCCCCCCC)CCCCCCCC] |
| VI- 186 | 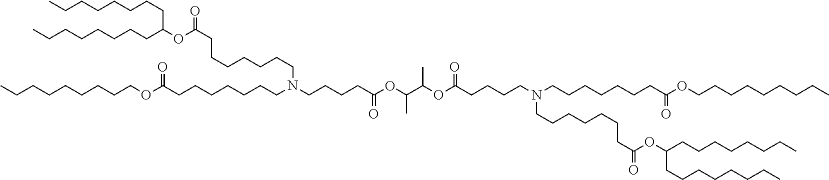 [SMILES: CCCCCCCCCOC(=O)CCCCCCCN(CCCCCCCC(=O)OC(CCCCCCCC)CCCCCCCC)CCCCC(=O)OC(C)C(C)OC(=O)CCCCN(CCCCCCCC(=O)OCCCCCCCCC)CCCCCCCC(=O)OC(CCCCCCCC)CCCCCCCC] |
| VI- 187 | 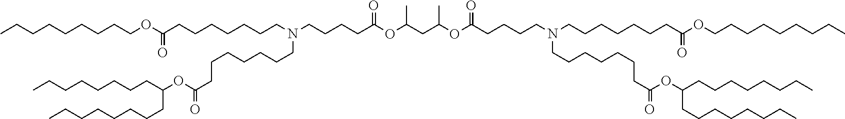 [SMILES: CCCCCCCCCOC(=O)CCCCCCCN(CCCCCCCC(=O)OC(CCCCCCCC)CCCCCCCC)CCCCC(=O)OC(C)CC(C)OC(=O)CCCCN(CCCCCCCC(=O)OCCCCCCCCC)CCCCCCCC(=O)OC(CCCCCCCC)CCCCCCCC] |
| VI- 188 | 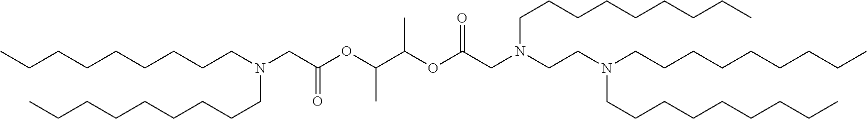 [SMILES: CCCCCCCCCN(CCCCCCCCC)CCN(CCCCCCCCC)CC(=O)OC(C)C(C)OC(=O)CN(CCCCCCCCC)CCCCCCCCC] |
| VI- 189 | 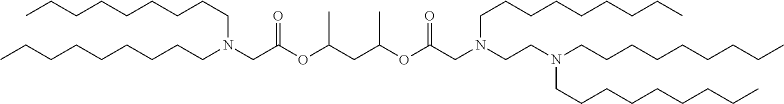 [SMILES: CCCCCCCCCN(CCCCCCCCC)CCN(CCCCCCCCC)CC(=O)OC(C)CC(C)OC(=O)CN(CCCCCCCCC)CCCCCCCCC] |

In some embodiments, the ionizable lipid is a compound selected from:

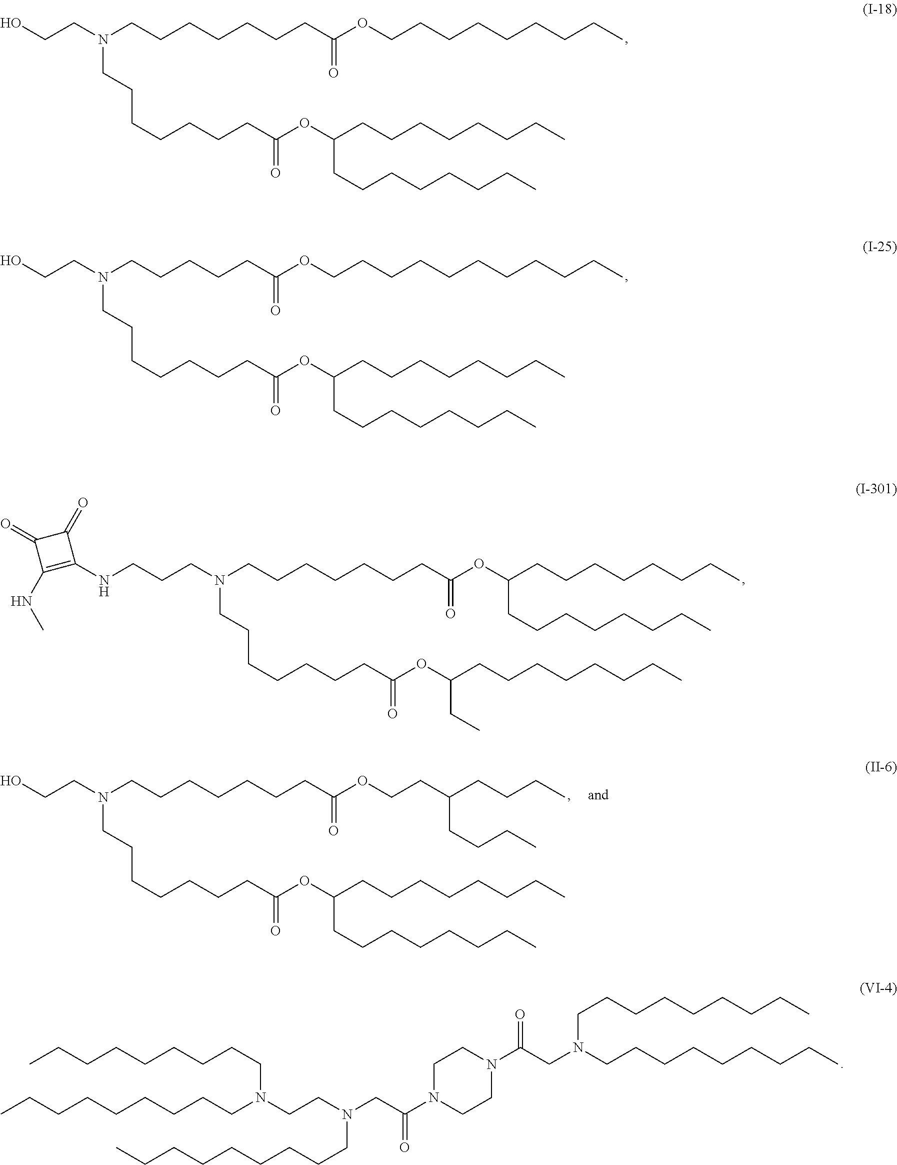

In some embodiments, the ionizable lipid is a lipid disclosed in Published International Patent Application Nos. WO/2017/049245, WO/2017/112865, WO/2018/170306, WO/2018/232120, WO/2021/055835, WO/2021/055833, and WO/2021/055849, each of which is incorporated by reference herein in its entirety.

### Structural Lipids

The lipid nanoparticle (e.g., an empty LNP or a loaded LNP) may include one or more structural lipids. Structural lipids can be selected from the group consisting of, but are not limited to, cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the structural lipid includes cholesterol and a corticosteroid (such as prednisolone, dexamethasone, prednisone, and hydrocortisone), or a combination thereof. In some embodiments, the structural lipid is:

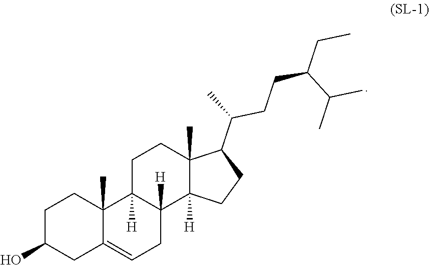

### Phospholipids

The lipid nanoparticle (e.g., an empty LNP or a loaded LNP) may include one or more phospholipids, such as one or more (poly)unsaturated lipids. Phospholipids may assemble into one or more lipid bilayers. In general, phospholipids may include a phospholipid moiety and one or more fatty acid moieties. For example, a phospholipid may be a lipid according to Formula (PhL-IV):

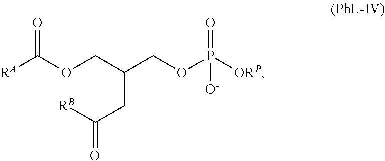

in which Rₚ represents a phospholipid moiety and R^{A} and R^{B} represent fatty acid moieties with or without unsaturation that may be the same or different. A phospholipid moiety may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a lipid nanoparticle (e.g., an empty LNP or a loaded LNP) to facilitate membrane permeation or cellular recognition or in conjugating a lipid nanoparticle (e.g., an empty LNP or a loaded LNP) to a useful component such as a targeting or imaging moiety (e.g., a dye).

Phospholipids useful in the compositions and methods may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidyethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), and mixtures thereof. In some embodiments, a lipid nanoparticle (e.g., an empty LNP or a loaded LNP) includes DSPC. In certain embodiments, a lipid nanoparticle (e.g., an empty LNP or a loaded LNP) includes DOPE. In some embodiments, a lipid nanoparticle (e.g., an empty LNP or a loaded LNP) includes both DSPC and DOPE.

### PEG Lipids

The lipid nanoparticle (e.g., an empty LNP or a loaded LNP) may include one or more PEG or PEG-modified lipids. Such species may be alternately referred to as PEGylated lipids. A PEG lipid is a lipid modified with polyethylene glycol. A PEG lipid may be selected from the non-limiting group consisting of PEG-modified phosphatidylethanolamines, PEG-modified phosphatidic acids, PEG-modified ceramides (PEG-CER), PEG-modified dialkylamines, PEG-modified diacylglycerols (PEG-DEG), PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In certain embodiments, the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol.

In certain embodiments, PEG lipid is selected from the group consisting of 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). For example, in some embodiments, the PEG lipid is PEG-DMG.

In certain embodiments, the PEG lipid is a compound of Formula (PL-I):

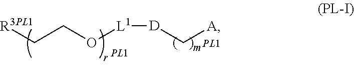

or a salt thereof, wherein:
R^{3PL1} is —OR^{OPL1}.
R^{OPL1} is hydrogen, optionally substituted alkyl, or an oxygen protecting group;
r^{PL1} is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{NPL1}), S, C(O), C(O)N(R^{NPL1}), NR^{NPL1}C(O), —C(O)O, OC(O), OC(O)O, OC(O)N(R^{NPL1}), NR^{NPL1}C(O)O, or NR^{NPL1}C(O)N(R^{NPL1});
   D is a moiety obtained by click chemistry or a moiety cleavable under physiological conditions;
   m^{PL1} is 0, 1, 2, 3, 4, 5, 6, 7, 8, 9, or 10;
A is of the formula:

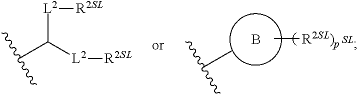

each instance of of L² is independently a bond or optionally substituted C₁₋₆ alkylene, wherein one methylene unit of the optionally substituted C₁₋₆ alkylene is optionally replaced with O, N(R^{NLP1}), S, C(O), C(O)N(R^{NLP1}), NR^{NPL1}C(O), C(O)O, OC(O), OC(O)O, —OC(O)N(R^{NPL1}), NR^{NPL1}C(O)O, or NR^{NPL1}C(O)N(R^{NPL1});
each instance of R^{25L} is independently optionally substituted C₁₋₃₀ alkyl, optionally substituted C₁₋₃₀ alkenyl, or optionally substituted C₁₋₃₀ alkynyl; optionally wherein one or more methylene units of R^{2SL} are independently replaced with optionally substituted carbocyclylene, optionally substituted heterocyclylene, optionally substituted arylene, optionally substituted heteroarylene, N(R^{NPL1}), O, S, C(O), C(O)N(R^{NPL1}), NR^{NPL1}C(O), —NR^{NPL1}C(O)N(R^{NPL1}), C(O)O, OC(O), OC(O)O, OC(O)N(R^{NPL1}), NR^{NPL1}C(O)O, C(O)S, —SC(O), C(═NR^{NPL1}), C(═NR^{NPL1})N(R^{NPL1}), NR^{NPL1}C(═NR^{NPL1}) NR^{NPL1}C(═NR^{NPL1})N(R^{NPL1}) C(S), C(S)N(R^{NPL1}), NR^{NPL1}C(S), NR^{NPL1}C(S)N(R^{NPL1}), S(O), OS(O), S(O)O, OS(O)O, —OS(O)₂, S(O)₂O, OS(O)₂O, N(R^{NPL1})S(O), S(O)N(R^{NPL1}), N(R^{NPL1})S(O)N(R^{NPL1}), —OS(O)N(R^{NPL1}), N(R^{NPL1})S(O)O, S(O)₂, N(R^{NPL1})S(O)₂, S(O)₂N(R^{NPL1}), —N(R^{NPL1})S(O)₂N(R^{NPL1}) OS(O)₂N(R^{NPL1}), or N(R^{NPL1})S(O)₂O;
each instance of R^{NPL1} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p^{SL} is 1 or 2.

In certain embodiments, the PEG lipid is a compound of Formula (PL-I-OH):

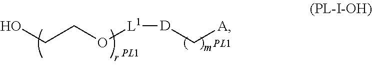

or a salt thereof.

In certain embodiments, the PEG lipid is a compound of Formula (PL-II-OH):

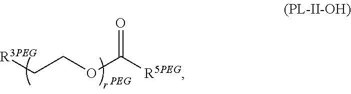

or a salt or isomer thereof, wherein:
R^{3PEG} is —OR^{O};
R^{O} is hydrogen, C₁₋₆ alkyl or an oxygen protecting group;
r^{PEG} is an integer between 1 and 100;
R^{5PEG} is C₁₀₋₄₀ alkyl, C₁₀₋₄₀ alkenyl, or C₁₀₋₄₀ alkynyl; and optionally one or more methylene groups of R⁵PEG are independently replaced with C₃₋₁₀ carbocyclylene, 4 to 10 membered heterocyclylene, C₆₋₁₀ arylene, 4 to 10 membered heteroarylene, —N(R^{NPEG})—, —O—, —S—, —C(O)—, —C(O)N(R^{NPEG})—, —NR^{NPEG}C(O)—, —NR^{NPEG}C(O)N(R^{NPEG})—, —C(O)O—, —OC(O)—, —OC(O)O—, —OC(O)N(R^{NPEG})—, —NR^{NPEG}C(O)O—, —C(O)S—, —SC(O)—, —C(═NR^{NPEG})—, —C(═NR^{NPEG})N(R^{NPEG})—, —NR^{NPEG}C(═NR^{NPEG})—, —NR^{NPEG}C(═NR^{NPEG})N(R^{NPEG})—, —C(S)—, —C(S)N(R^{NPEG})—, —NR^{NPEG}C(S)—, —NR^{NPEG}C(S)N(R^{NPEG})—, —S(O)—, —OS(O)—, —S(O)O—, —OS(O)O—, —OS(O)₂—, —S(O)₂O—, —OS(O)₂O—, —N(R^{NPEG})S(O)—, —S(O)N(R^{NPEG})—, —N(R^{NPEG})S(O)N(R^{NPEG})—, —OS(O)N(R^{NPEG})—, —N(R^{NPEG})S(O)O—, —S(O)₂—, —N(R^{NPEG})S(O)₂—, —S(O)₂N(R^{NPEG})—, —N(R^{NPEG})S(O)₂N(R^{NPEG})—, —OS(O)₂N(R^{NPEG})—, or —N(R^{NPEG})S(O)₂O—; and each instance of R^{NPEG} is independently hydrogen, C₁₋₆ alkyl, or a nitrogen protecting group.

In certain embodiments, in the PEG lipid of Formula (PL-II-OH), r is an integer between 40 and 50. For example, r is selected from the group consisting of 40, 41, 42, 43, 44, 45, 46, 47, 48, 49 and 50. For example, r is 45.

In certain embodiments, in the PEG lipid of Formula (PL-II-OH), R⁵ is C₁₇ alkyl.

In certain embodiments, the PEG lipid is a compound of Formula (PL-II):

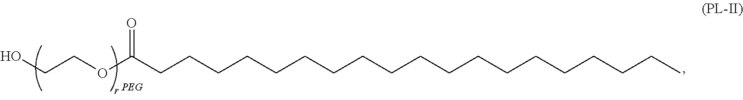

wherein r is an integer between 1 and 100.

In certain embodiments, the PEG lipid is a compound of Formula (PEG-1):

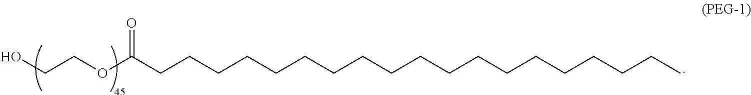

In certain embodiments, the PEG lipid is a compound of Formula (PL-III):

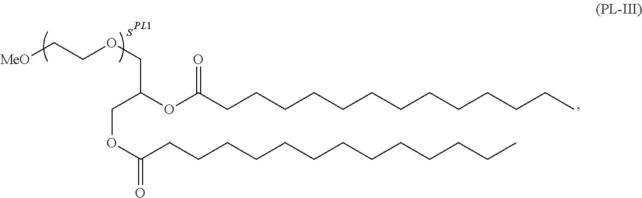

or a salt or isomer thereof, wherein s^{PL1} is an integer between 1 and 100.

In certain embodiments, the PEG lipid is a compound of following formula:

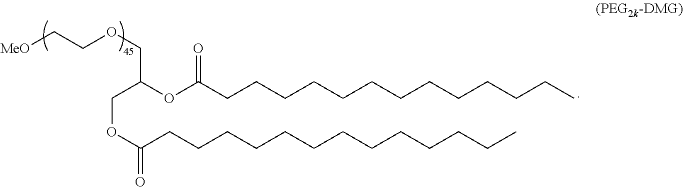

In certain embodiments, the incorporation of a lipid of formula (PL-I), PL-I-OH), (PL-II), (PL-II-OH), (PL-III), PEG₂ₖ-DMG, or PEG-1 in the nanoparticle formulation can improve the pharmacokinetics and/or biodistribution of the lipid nanoparticle formulations. For example, incorporation of a lipid of formula (PL-II-OH), (PL-IIa-OH), (PL-II), or PEG-1in the nanoparticle formulation can reduce the accelerated blood clearance (ABC) effect.

### Adjuvants

In some embodiments, a lipid nanoparticle (e.g., an empty LNP or a loaded LNP) that includes one or more lipids described herein may further include one or more adjuvants, e.g., Glucopyranosyl Lipid Adjuvant (GLA), CpG oligodeoxynucleotides (e.g., Class A or B), poly(I.C), aluminum hydroxide, and Pam3CSK4.

### Therapeutic Agents

Lipid nanoparticles (e.g., empty LNPs or loaded LNPs) may include one or more therapeutic and/or prophylactics. The disclosure features methods of delivering a therapeutic and/or prophylactic to a mammalian cell or organ, producing a polypeptide of interest in a mammalian cell, and treating a disease or disorder in a mammal in need thereof comprising administering to a mammal and/or contacting a mammalian cell with a lipid nanoparticle (e.g., an empty LNP or a loaded LNP) including a therapeutic and/or prophylactic.

Therapeutic and/or prophylactics include biologically active substances and are alternately referred to as “active agents.” A therapeutic and/or prophylactic may be a substance that, once delivered to a cell or organ, brings about a desirable change in the cell, organ, or other bodily tissue or system. Such species may be useful in the treatment of one or more diseases, disorders, or conditions. In some embodiments, a therapeutic and/or prophylactic is a small molecule drug useful in the treatment of a particular disease, disorder, or condition.

In some embodiments, a therapeutic and/or prophylactic is a vaccine, a compound (e.g., a polynucleotide or nucleic acid molecule that encodes a protein or polypeptide or peptide or a protein or polypeptide or protein) that elicits an immune response, and/or another therapeutic and/or prophylactic. Vaccines include compounds and preparations that are capable of providing immunity against one or more conditions related to infectious diseases and can include mRNAs encoding infectious disease derived antigens and/or epitopes. Vaccines also include compounds and preparations that direct an immune response against cancer cells and can include mRNAs encoding tumor cell derived antigens, epitopes, and/or neoepitopes. In some embodiments, a vaccine and/or a compound capable of eliciting an immune response is administered intramuscularly via a composition of the disclosure.

In other embodiments, a therapeutic and/or prophylactic is a protein, for example a protein needed to augment or replace a naturally-occurring protein of interest. Such proteins or polypeptides may be naturally occurring, or may be modified using methods known in the art, e.g., to increase half life. Exemplary proteins are intracellular, transmembrane, or secreted.

### Polynucleotides and Nucleic Acids

In some embodiments, the therapeutic agent is an agent that enhances (i.e., increases, stimulates, upregulates) protein expression. Non-limiting examples of types of therapeutic agents that can be used for enhancing protein expression include RNAs, mRNAs, dsRNAs, CRISPR/Cas9 technology, ssDNAs and DNAs (e.g., expression vectors). The agent that upregulates protein expression may upregulate expression of a naturally occurring or non-naturally occurring protein (e.g., a chimeric protein that has been modified to improve half life, or one that comprises desirable amino acid changes). Exemplary proteins include intracellular, transmembrane, or secreted proteins, peptides, or polypeptides.

In some embodiments, the therapeutic agent is a DNA therapeutic agent. The DNA molecule can be a double-stranded DNA, a single-stranded DNA (ssDNA), or a molecule that is a partially double-stranded DNA, i.e., has a portion that is double-stranded and a portion that is single-stranded. In some cases the DNA molecule is triple-stranded or is partially triple-stranded, i.e., has a portion that is triple stranded and a portion that is double stranded. The DNA molecule can be a circular DNA molecule or a linear DNA molecule.

A DNA therapeutic agent can be a DNA molecule that is capable of transferring a gene into a cell, e.g., that encodes and can express a transcript. In other embodiments, the DNA molecule is a synthetic molecule, e.g., a synthetic DNA molecule produced in vitro. In some embodiments, the DNA molecule is a recombinant molecule. Non-limiting exemplary DNA therapeutic agents include plasmid expression vectors and viral expression vectors.

The DNA therapeutic agents described herein, e.g., DNA vectors, can include a variety of different features. The DNA therapeutic agents described herein, e.g., DNA vectors, can include a non-coding DNA sequence. For example, a DNA sequence can include at least one regulatory element for a gene, e.g., a promoter, enhancer, termination element, polyadenylation signal element, splicing signal element, and the like. In some embodiments, the non-coding DNA sequence is an intron. In some embodiments, the non-coding DNA sequence is a transposon. In some embodiments, a DNA sequence described herein can have a non-coding DNA sequence that is operatively linked to a gene that is transcriptionally active. In other embodiments, a DNA sequence described herein can have a non-coding DNA sequence that is not linked to a gene, i.e., the non-coding DNA does not regulate a gene on the DNA sequence.

In some embodiments, in the loaded LNP of the disclosure, the one or more therapeutic and/or prophylactic agents is a nucleic acid. In some embodiments, the one or more therapeutic and/or prophylactic agents is selected from the group consisting of a ribonucleic acid (RNA) and a deoxyribonucleic acid (DNA).

For example, in some embodiments, when the therapeutic and/or prophylactic agents is a DNA, the DNA is selected from the group consisting of a double-stranded DNA, a single-stranded DNA (ssDNA), a partially double-stranded DNA, a triple stranded DNA, and a partially triple-stranded DNA. In some embodiments, the DNA is selected from the group consisting of a circular DNA, a linear DNA, and mixtures thereof.

In some embodiments, in the loaded LNP of the disclosure, the one or more therapeutic and/or prophylactic agents is selected from the group consisting of a plasmid expression vector, a viral expression vector, and mixtures thereof.

For example, in some embodiments, when the therapeutic and/or prophylactic agents is a RNA, the RNA is selected from the group consisting of a single-stranded RNA, a double-stranded RNA (dsRNA), a partially double-stranded RNA, and mixtures thereof. In some embodiments, the RNA is selected from the group consisting of a circular RNA, a linear RNA, and mixtures thereof.

For example, in some embodiments, when the therapeutic and/or prophylactic agents is a RNA, the RNA is selected from the group consisting of a short interfering RNA (siRNA), an asymmetrical interfering RNA (aiRNA), a RNA interference (RNAi) molecule, a microRNA (miRNA), an antagomir, an antisense RNA, a ribozyme, a Dicer-substrate RNA (dsRNA), a small hairpin RNA (shRNA), a messenger RNA (mRNA), locked nucleic acids (LNAs) and CRISPR/Cas9 technology, and mixtures thereof.

For example, in some embodiments, when the therapeutic and/or prophylactic agents is a RNA, the RNA is selected from the group consisting of a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and mixtures thereof.

In some embodiments, the one or more therapeutic and/or prophylactic agents is an mRNA. In some embodiments, the one or more therapeutic and/or prophylactic agents is a modified mRNA (mmRNA).

In some embodiments, the one or more therapeutic and/or prophylactic agents is an mRNA that incorporates a micro-RNA binding site (miR binding site). Further, in some embodiments, an mRNA includes one or more of a stem loop, a chain terminating nucleoside, a polyA sequence, a polyadenylation signal, and/or a 5′ cap structure.

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. In some embodiments, all uracils or uridines are modified. When all nucleobases, nucleosides, or nucleotides are modified, e.g., all uracils or uridines, the mRNA can be referred to as “fully modified”, e.g., for uracil or uridine.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m27,03′GpppG, m27,03′GppppG, m27,02′GppppG, m7Gpppm7G, m73′dGpppG, m27,03′GpppG, m27,03′GppppG, and m27,02′GppppG.

An mRNA may instead or additionally include a chain terminating nucleoside. For example, a chain terminating nucleoside may include those nucleosides deoxygenated at the 2′ and/or 3′ positions of their sugar group. Such species may include 3′ deoxyadenosine (cordycepin), 3′ deoxyuridine, 3′ deoxycytosine, 3′ deoxyguanosine, 3′ deoxythymine, and 2′,3′ dideoxynucleosides, such as 2′,3′ dideoxyadenosine, 2′,3′ dideoxyuridine, 2′,3′ dideoxycytosine, 2′,3′ dideoxyguanosine, and 2′,3′ dideoxythymine. In some embodiments, incorporation of a chain terminating nucleotide into an mRNA, for example at the 3′-terminus, may result in stabilization of the mRNA.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A poly A sequence may also comprise stabilizing nucleotides or analogs. For example, a poly A sequence can include deoxythymidine, e.g., inverted (or reverse linkage) deoxythymidine (dT), as a stabilizing nucleotide or analog. Details on using inverted dT and other stabilizing poly A sequence modifications can be found, for example, in WO2017/049275 A2, the content of which is incorporated herein by reference. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site. MicroRNA binding sites (or miR binding sites) can be used to regulate mRNA expression in various tissues or cell types. In exemplary embodiments, miR binding sites are engineered into 3′ UTR sequences of an mRNA to regulate, e.g., enhance degradation of mRNA in cells or tissues expressing the cognate miR. Such regulation is useful to regulate or control “off-target” expression ir mRNAs, i.e., expression in undesired cells or tissues in vivo. Details on using mir binding sites can be found, for example, in WO 2017/062513 A2, the content of which is incorporated herein by reference.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine(τm5s2U), 1-taurinomethyl-4-thio-pseudouridine, 5-methyl-uridine (m5U, i.e., having the nucleobase deoxythymine), 1-methyl-pseudouridine (m1ψ), 5-methyl-2-thio-uridine (m5s2U), 1-methyl-4-thio-pseudouridine (m1s4ψ), 4-thio-1-methyl-pseudouridine, 3-methyl-pseudouridine (m3yr), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (Wm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include α-thio-adenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, α-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include α-thio-guanosine, inosine (I), 1-methyl-inosine (mlI), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ0), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m22G), N2,7-dimethyl-guanosine (m2,7G), N2, N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 2′-O-ribosylguanosine (phosphate) (Gr(p)), 1-thio-guanosine, 06-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is pseudouridine (ψ), N1-methylpseudouridine (m1ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases.) In some embodiments, the modified nucleobase is N1-methylpseudouridine (m1ψ) and the mRNA of the disclosure is fully modified with N1-methylpseudouridine (m1ψ). In some embodiments, N1-methylpseudouridine (m1ψ) represents from 75-100% of the uracils in the mRNA. In some embodiments, N1-methylpseudouridine (m1ψ) represents 100% of the uracils in the mRNA.

In some embodiments, the modified nucleobase is a modified cytosine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified adenine. Exemplary nucleobases and nucleosides having a modified adenine include 7-deaza-adenine, 1-methyl-adenosine (m1A), 2-methyl-adenine (m2A), N6-methyl-adenosine (m6A). In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is 1-methyl-pseudouridine (m1ψ), 5-methoxy-uridine (mo5U), 5-methyl-cytidine (m5C), pseudouridine (ψ), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases.)

In some embodiments, the mRNA comprises pseudouridine (ψ). In some embodiments, the mRNA comprises pseudouridine (ψ) and 5-methyl-cytidine (m5C). In some embodiments, the mRNA comprises 1-methyl-pseudouridine (m1ψ). In some embodiments, the mRNA comprises 1-methyl-pseudouridine (m1ψ) and 5-methyl-cytidine (m5C). In some embodiments, the mRNA comprises 2-thiouridine (s2U). In some embodiments, the mRNA comprises 2-thiouridine and 5-methyl-cytidine (m5C). In some embodiments, the mRNA comprises 5-methoxy-uridine (mo5U). In some embodiments, the mRNA comprises 5-methoxy-uridine (mo5U) and 5-methyl-cytidine (m5C). In some embodiments, the mRNA comprises 2′-O-methyl uridine. In some embodiments, the mRNA comprises 2′-O-methyl uridine and 5-methyl-cytidine (m5C). In some embodiments, the mRNA comprises comprises N6-methyl-adenosine (m6A). In some embodiments, the mRNA comprises N6-methyl-adenosine (m6A) and 5-methyl-cytidine (m5C).

In certain embodiments, an mRNA of the disclosure is uniformly modified (i.e., fully modified, modified through-out the entire sequence) for a particular modification. For example, an mRNA can be uniformly modified with N1-methylpseudouridine (m1ψ) or 5-methyl-cytidine (m5C), meaning that all uridines or all cytosine nucleosides in the mRNA sequence are replaced with N1-methylpseudouridine (m1ψ) or 5-methyl-cytidine (m5C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

The mmRNAs of the disclosure can include a combination of modifications to the sugar, the nucleobase, and/or the internucleoside linkage. These combinations can include any one or more modifications described herein.

Where a single modification is listed, the listed nucleoside or nucleotide represents 100 percent of that A, U, G or C nucleotide or nucleoside having been modified. Where percentages are listed, these represent the percentage of that particular A, U, G or C nucleobase triphosphate of the total amount of A, U, G, or C triphosphate present. For example, the combination: 25% 5-Aminoallyl-CTP+75% CTP/25% 5-Methoxy-UTP+75% UTP refers to a polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed then the naturally occurring ATP, UTP, GTP and/or CTP is used at 100% of the sites of those nucleotides found in the polynucleotide. In this example all of the GTP and ATP nucleotides are left unmodified.

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, CA) and/or proprietary methods. In some embodiments, the mRNA sequence is optimized using optimization algorithms, e.g., to optimize expression in mammalian cells or enhance mRNA stability.

In certain embodiments, the present disclosure includes polynucleotides having at least 80%, at least 85%, at least 90%, at least 95%, at least 98%, or at least 99% sequence identity to any of the polynucleotide sequences described herein.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In some embodiments, mRNAs are made using IVT enzymatic synthesis methods. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme.

Either enzymatic or chemical ligation methods may be used to conjugate polynucleotides or their regions with different functional moieties, such as targeting or delivery agents, fluorescent labels, liquids, nanoparticles, etc. Therapeutic Agents for Reducing Protein Expression

In some embodiments, the therapeutic agent is a therapeutic agent that reduces (i.e., decreases, inhibits, downregulates) protein expression. Non-limiting examples of types of therapeutic agents that can be used for reducing protein expression include mRNAs that incorporate a micro-RNA binding site(s) (miR binding site), microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNAs), locked nucleic acids (LNAs) and CRISPR/Cas9 technology.

### Peptide Polypeptide Therapeutic Agents

In some embodiments, the therapeutic agent is a peptide therapeutic agent. In some embodiments the therapeutic agent is a polypeptide therapeutic agent.

In some embodiments, the peptide or polypeptide is naturally-derived, e.g., isolated from a natural source. In other embodiments, the peptide or polypeptide is a synthetic molecule, e.g., a synthetic peptide or polypeptide produced in vitro. In some embodiments, the peptide or polypeptide is a recombinant molecule. In some embodiments, the peptide or polypeptide is a chimeric molecule. In some embodiments, the peptide or polypeptide is a fusion molecule. In some embodiments, the peptide or polypeptide therapeutic agent of the composition is a naturally occurring peptide or polypeptide. In some embodiments, the peptide or polypeptide therapeutic agent of the composition is a modified version of a naturally occurring peptide or polypeptide (e.g., contains less than 3, less than 5, less than 10, less than 15, less than 20, or less than 25 amino substitutions, deletions, or additions compared to its wild type, naturally occurring peptide or polypeptide counterpart).

In some embodiments, in the loaded LNP of the disclosure, the one or more therapeutic and/or prophylactic agents is a polynucleotide or a polypeptide.

### Other Components

A lipid nanoparticle (e.g., an empty LNP or a loaded LNP) may include one or more components in addition to those described in the preceding sections. For example, a lipid nanoparticle (e.g., an empty LNP or a loaded LNP) may include one or more small hydrophobic molecules such as a vitamin (e.g., vitamin A or vitamin E) or a sterol.

Lipid nanoparticles (e.g., empty LNPs or loaded LNPs) may also include one or more permeability enhancer molecules, carbohydrates, polymers, surface altering agents, or other components. Carbohydrates may include simple sugars (e.g., glucose) and polysaccharides (e.g., glycogen and derivatives and analogs thereof).

A polymer may be included in and/or used to encapsulate or partially encapsulate a nanoparticle composition. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, polyoxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a lipid nanoparticle (e.g., an empty LNP or a loaded LNP) (e.g., by coating, adsorption, covalent linkage, or other process).

A lipid nanoparticle (e.g., an empty LNP or a loaded LNP) may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a lipid nanoparticle (e.g., an empty LNP or a loaded LNP) may also be conjugated with one or more useful antibodies. Functional groups and conjugates useful in targeted cell delivery, imaging, and membrane permeation are well known in the art.

In addition to these components, lipid nanoparticles (e.g., empty LNPs or loaded LNPs) may include any substance useful in pharmaceutical compositions. For example, the lipid nanoparticle (e.g., an empty LNP or a loaded LNP) may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included.

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent may be starch (e.g. cornstarch and starch paste); gelatin; sugars (e.g. sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

### Production of Nanoparticle Compositions

In some embodiments, nanoparticles comprising lipids of the disclosure are prepared by combining a cationic lipid according to Formula (I), an ionizable lipid according to Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), a phospholipid (e.g., DSPC), a PEG lipid (e.g., 1,2-dimyristoyl-sn-glycerol methoxypolyethylene glycol, also known as PEG-DMG, e.g., PEG₂ₖ-DMG, or PEG-1), and a structural lipid (e.g., cholesterol) using, for e.g., ethanol drop nanoprecipitation followed by solvent exchange into suitable aqueous buffer using dialysis.

### Characterization of Nanoparticle Compositions

Zeta potential measures the electrokinetic potential in colloidal dispersions. The magnitude of the zeta potential indicates the degree of electrostatic repulsion between adjacent, similarly charged particles in the dispersion. Zeta potential can be measured on a Wyatt Technologies Mobius Zeta Potential instrument. This instrument characterizes the mobility and zeta potential by the principle of “Massively Parallel Phase Analysis Light Scattering” or MP-PALS. Without wishing to be bound by theory, this measurement is more sensitive and less stress inducing than ISO Method 13099-1:2012 which only uses one angle of detection and required higher voltage for operation. In some embodiments, the zeta potential of an empty or loaded LNP of the disclosure is measured using an instrument employing the principle of MP-PALS.

Ultraviolet-visible spectroscopy can be used to determine the concentration of a therapeutic and/or prophylactic (e.g., RNA) in loaded LNPs. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, CA). The concentration of therapeutic and/or prophylactic in the loaded LNP can be calculated based on the extinction coefficient of the therapeutic and/or prophylactic used in the composition and on the difference between the absorbance at a wavelength of, for example, 260 nm and the baseline value at a wavelength of, for example, 330 nm.

For loaded LNPs including an RNA, a QUANT-IT™ RIBOGREEN® RNA assay (Invitrogen Corporation Carlsbad, CA) can be used to evaluate the encapsulation of an RNA by the LNP. The samples are diluted to a concentration of approximately 5 g/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2% Triton X-100 solution is added to the wells. The plate is incubated at a temperature of 37° C. for 15 minutes. The RIBOGREEN® reagent is diluted 1:100 in TE buffer, and 100 μL of this solution is added to each well. The fluorescence intensity can be measured using a fluorescence plate reader (Wallac Victor 1420 Multilablel Counter; Perkin Elmer, Waltham, MA) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

### In Vivo Formulation Studies

In order to monitor how effectively various nanoparticle compositions deliver therapeutic and/or prophylactics to targeted cells, different nanoparticle compositions including a particular therapeutic and/or prophylactic (for example, a modified or naturally occurring RNA such as an mRNA) are prepared and administered to animal populations. Animals (e.g., mice, rats, or non-human primates) are intravenously, intramuscularly, intraarterially, or intratumorally administered a single dose including a nanoparticle composition comprising a lipid of the disclosure and an mRNA expressing a protein, e.g., OX40L, or tdTomato. A control composition including PBS may also be employed.

Upon administration of nanoparticle compositions to an animal, dose delivery profiles, dose responses, and toxicity of particular formulations and doses thereof can be measured by enzyme-linked immunosorbent assays (ELISA), bioluminescent imaging, or other methods. For nanoparticle compositions including mRNA, time courses of protein expression can also be evaluated. Samples collected from the animals for evaluation may include blood, sera, and tissue (for example, muscle tissue from the site of an intramuscular injection and internal tissue); sample collection may involve sacrifice of the animals.

Nanoparticle compositions (e.g. loaded LNPs) including mRNA are useful in the evaluation of the efficacy and usefulness of various formulations for the delivery of therapeutic and/or prophylactics. Higher levels of protein expression induced by administration of a composition including an mRNA will be indicative of higher mRNA translation and/or nanoparticle composition mRNA delivery efficiencies. As the non-RNA components are not thought to affect translational machineries themselves, a higher level of protein expression is likely indicative of a higher efficiency of delivery of the therapeutic and/or prophylactic by a given nanoparticle composition relative to other nanoparticle compositions or the absence thereof.

### Formulations

Lipid nanoparticles (e.g., empty LNPs or loaded LNPs) may include a lipid component and one or more additional components, such as a therapeutic and/or prophylactic. A lipid nanoparticle (e.g., an empty LNP or a loaded LNP) may be designed for one or more specific applications or targets. The elements of a lipid nanoparticle (e.g., an empty LNP or a loaded LNP) may be selected based on a particular application or target, and/or based on the efficacy, toxicity, expense, ease of use, availability, or other feature of one or more elements. Similarly, the particular formulation of a nanoparticle composition may be selected for a particular application or target according to, for example, the efficacy and toxicity of particular combinations of elements.

In some embodiments, the lipid component of a lipid nanoparticle composition (e.g., an empty LNP or a loaded LNP) includes, for example, a cationic lipid according to Formula (I), an ionizable lipid according to Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), a phospholipid (such as an unsaturated lipid, e.g., DOPE or DSPC), a PEG lipid, and a structural lipid. The elements of the lipid component may be provided in specific fractions.

In some embodiments, the lipid component of an empty LNP or a loaded LNP includes a cationic lipid according to Formula (I), an ionizable lipid according to Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), a phospholipid, a PEG lipid, and a structural lipid. In certain embodiments, the lipid component of the nanoparticle composition includes from about 20 mol % to about 40 mol % cationic lipid according to Formula (I), from about 15 mol % to about 40 mol % ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), from about 0 mol % to about 30 mol % phospholipid, from about 18.5 mol % to about 48.5 mol % structural lipid, and from about 0 mol % to about 10 mol % of PEG lipid, provided that the total mol % does not exceed 100%. In some embodiments, the lipid component of the nanoparticle composition includes from about 20 mol % to about 40 mol % cationic lipid according to Formula (I), from about about 20 mol % to about 25 mol % ionizable lipid of Formula (TL-A), (TL-B), (TL-C), (TL-D), (IL-I), (IL-IA), (IL-IB), (IL-IC), (IL-IIA), (IL-IIAX), (IL-IIB), (IL-IIC), (IL-III), or (IL-IIIA), from about 5 mol % to about 25 mol % phospholipid, from about 30 mol % to about 40 mol % structural lipid, and from about 0 mol % to about 10 mol % of PEG lipid.

In some embodiments an empty lipid nanoparticle (empty LNP) comprises a cationic lipid of Formula (I), an ionizable lipid according to Formula (TL-A), (IL-B), (TL-C), (TL-D), (IL-I), (IL-IA), (IL-IB), (IL-IC), (IL-IIA), (IL-IIAX), (IL-IIB), (IL-IIC), (IL-III), or (IL-IIIA), a phospholipid, a structural lipid, and a PEG lipid.

In some embodiments a loaded lipid nanoparticle (loaded LNP) comprises a cationic lipid of Formula (I), an ionizable lipid according to Formula (TL-A), (IL-B), (TL-C), (IL-D), (IL-I), (IL-IA), (IL-IB), (IL-IC), (IL-IIA), (IL-IIAX), (IL-IIB), (IL-IIC), (IL-III), or (IL-IIIA), a phospholipid, a structural lipid, a PEG lipid, and one or more therapeutic and/or prophylactic agents.

In some embodiments, the empty LNP or loaded LNP comprises the cationic lipid of Formula (I), in an amount from about 20 mol % to about 40 mol %.

In some embodiments, the empty LNP or loaded LNP comprises an ionizable lipid of Formula (IL-A), (IL-B), (IL-C), (TL-D), (IL-I), (IL-IA), (IL-IB), (IL-IC), (IL-IIA), (IL-IIAX), (IL-IIB), (IL-IIC), (IL-III), or (IL-IIIA), in an amount from about 15 mol % to about 40 mol %.

In some embodiments, the empty LNP or loaded LNP comprises the phospholipid in an amount from about 0 mol % to about 20 mol %. For example, in some embodiments, the empty LNP or loaded LNP comprises DSPC in an amount from about 0 mol % to about 20 mol 0.

In some embodiments, the empty LNP or loaded LNP comprises the structural lipid in an amount from about 30 mol % to about 50 mol %. For example, in some embodiments, the empty LNP or loaded LNP comprises cholesterol in an amount from about 30 mol % to about 50 mol %.

In some embodiments, the empty LNP or loaded LNP comprises the PEG lipid in an amount from about 0 mol % to about 5 mol %. For example, in some embodiments, the empty LNP or loaded LNP comprises PEG-1 or PEG₂ₖ-DMG in an amount from about 0 mol % to about 5 mol %.

In some embodiments, the empty LNP or loaded LNP comprises about 20 mol % to about 40 mol % of the cationic lipid of Formula (I), about 15 mol % to about 40 mol % of an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), about 0 mol % to about 20 mol % phospholipid, about 30 mol % to about 50 mol % structural lipid, and about 0 mol % to about 5 mol % PEG lipid.

In some embodiments, the empty LNP or loaded LNP comprises about 20 mol % to about 40 mol % of the cationic lipid of Formula (I), about 15 mol % to about 40 mol % of the ionizable lipid of Formula (TL-A), (TL-B), (TL-C), (TL-D), (IL-I), (IL-IA), (IL-IB), (IL-IC), (IL-IIA), (IL-IIAX), (IL-IIB), (IL-IIC), (IL-III), or (IL-IIIA), about 0 mol % to about 20 mol % DSPC, about 30 mol % to about 50 mol % cholesterol, and about 0 mol % to about 5 mol % PEG₂ₖ-DMG. In some embodiments, the empty LNP or loaded LNP comprises about 20 mol % to about 40 mol % of a lipid of Table 1, about 15 mol % to about 40 mol % of a lipid of Tables IL-1 to IL-7, about 0 mol % to about 20 mol % DSPC, about 30 mol % to about 50 mol % cholesterol, and about 0 mol % to about 5 mol % PEG₂ₖ-DMG.

In some embodiments, the empty LNP or loaded LNP comprises about 20 mol % to about 40 mol % of the cationic lipid of Formula (I), about 15 mol % to about 40 mol % of the ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), about 0 mol % to about 20 mol % DSPC, about 30 mol % to about 50 mol % cholesterol, and about 0 mol % to about 5 mol % PEG-1. In some embodiments, the empty LNP or loaded LNP comprises about 20 mol % to about 40 mol % of a lipid of Table 1, about 15 mol % to about 40 mol % of a lipid of Tables IL-1 to IL-7, about 0 mol % to about 20 mol % DSPC, about 30 mol % to about 50 mol % cholesterol, and about 0 mol % to about 5 mol % PEG-1.

In some embodiments, the empty LNP or loaded LNP comprises a cationic lipid of Formula (I), an ionizable lipid of Formula (TL-A), (TL-B), (TL-C), (TL-D), (IL-I), (IL-IA), (IL-IB), (IL-IC), (IL-IIA), (IL-IIAX), (IL-IIB), (IL-IIC), (IL-III), or (IL-IIIA), a phospholipid, a structural lipid, and a PEG lipid, wherein the phospholipid is DSPC and the structural lipid is cholesterol. In some embodiments, the empty LNP or loaded LNP comprises a cationic lipid of Table 1, an ionizable lipid of Tables IL-1 to IL-7, a phospholipid, a structural lipid, and a PEG lipid, wherein the phospholipid is DSPC and the structural lipid is cholesterol.

In some embodiments, the empty LNP or loaded LNP comprises a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), a phospholipid, a structural lipid, and a PEG lipid, wherein the structural lipid is cholesterol and the PEG lipid is PEG₂ₖ-DMG. In some embodiments, the empty LNP or loaded LNP comprises a lipid of Table 1, an ionizable lipid of Tables IL-1 to IL-7, a phospholipid, a structural lipid, and a PEG lipid, wherein the structural lipid is cholesterol and the PEG lipid is PEG₂ₖ-DMG.

In some embodiments, the empty LNP or loaded LNP comprises a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), a phospholipid, a structural lipid, and a PEG lipid, wherein the structural lipid is cholesterol and the PEG lipid is PEG-1. In some embodiments, the empty LNP or loaded LNP comprises a cationic lipid of Table 1, an ionizable lipid of Tables IL-1 to IL-7, a phospholipid, a structural lipid, and a PEG lipid, wherein the structural lipid is cholesterol and the PEG lipid is PEG-1.

In some embodiments, the empty LNP or loaded LNP comprises a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-ITB), (IL-IIC), (IL-III), or (IL-IIIA), a phospholipid, a structural lipid, and a PEG lipid, wherein the phospholipid is DSPC and the PEG lipid is PEG₂ₖ-DMG. In some embodiments, the empty LNP or loaded LNP comprises a lipid of Table 1, an ionizable lipid of Tables IL-1 to IL-7, a phospholipid, a structural lipid, and a PEG lipid, wherein the phospholipid is DSPC and the PEG lipid is PEG₂ₖ-DMG.

In some embodiments, the empty LNP or loaded LNP comprises a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), a phospholipid, a structural lipid, and a PEG lipid, wherein the phospholipid is DSPC and the PEG lipid is PEG-1. In some embodiments, the empty LNP or loaded LNP comprises a lipid of Table 1, an ionizable lipid of Tables IL-1 to IL-7, a phospholipid, a structural lipid, and a PEG lipid, wherein the phospholipid is DSPC and the PEG lipid is PEG-1.

In some embodiments, the empty LNP or loaded LNP comprises a lipid of Formula (I), an ionizable lipid of Formula (TL-A), (TL-B), (TL-C), (TL-D), (IL-I), (IL-IA), (IL-IB), (IL-IC), (IL-IIA), (IL-IIAX), (IL-IIB), (IL-IIC), (IL-III), or (IL-IIIA), a phospholipid, a structural lipid, and a PEG lipid, wherein the phospholipid is DSPC, the structural lipid is cholesterol, and the PEG lipid is PEG₂ₖ-DMG. In some embodiments, the empty LNP or loaded LNP comprises a cationic lipid of Table 1, an ionizable lipid of Tables IL-1 to IL-7, a phospholipid, a structural lipid, and a PEG lipid, wherein the phospholipid is DSPC, the structural lipid is cholesterol, and the PEG lipid is PEG₂ₖ-DMG. In some embodiments, the empty LNP or loaded LNP comprises a cationic lipid of Table 1, an ionizable lipid of Tables IL-1 to IL-7, a phospholipid, a structural lipid, and a PEG lipid, wherein the phospholipid is DSPC, the structural lipid is cholesterol, and the PEG lipid is PEG₂ₖ-DMG.

In some embodiments, the empty LNP or loaded LNP comprises a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), a phospholipid, a structural lipid, and a PEG lipid, wherein the phospholipid is DSPC, the structural lipid is cholesterol, and the PEG lipid is PEG-1. In some embodiments, the empty LNP or loaded LNP comprises a cationic lipid of Table 1, an ionizable lipid of Tables IL-1 to IL-7, a phospholipid, a structural lipid, and a PEG lipid, wherein the phospholipid is DSPC, the structural lipid is cholesterol, and the PEG lipid is PEG-1.

Lipid nanoparticles (e.g., empty LNPs or loaded LNPs) may be designed for one or more specific applications or targets. For example, a nanoparticle composition may be designed to deliver a therapeutic and/or prophylactic such as an RNA to a particular cell, tissue, organ, or system or group thereof in a mammal's body. Physiochemical properties of lipid nanoparticles (e.g., empty LNPs or loaded LNPs) may be altered in order to increase selectivity for particular bodily targets. For instance, particle sizes may be adjusted based on the fenestration sizes of different organs. The therapeutic and/or prophylactic included in a nanoparticle composition may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a nanoparticle composition may include an mRNA encoding a polypeptide of interest capable of being translated within a cell to produce the polypeptide of interest. Such a composition may be designed to be specifically delivered to a particular organ. In some embodiments, a composition may be designed to be specifically delivered to a mammalian liver. In some embodiments, a composition may be designed to be specifically delivered to a mammalian lung.

The amount of a therapeutic and/or prophylactic in a nanoparticle composition may depend on the size, composition, desired target and/or application, or other properties of the nanoparticle composition as well as on the properties of the therapeutic and/or prophylactic. For example, the amount of an RNA useful in a nanoparticle composition may depend on the size, sequence, and other characteristics of the RNA. The relative amounts of a therapeutic and/or prophylactic and other elements (e.g., lipids) in a nanoparticle composition may also vary. In some embodiments, the wt/wt ratio of the lipid component to a therapeutic and/or prophylactic in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1.

The amount of a therapeutic and/or prophylactic in a nanoparticle composition may, for example, be measured using absorption spectroscopy (e.g., ultraviolet-visible spectroscopy).

In some embodiments, a nanoparticle composition includes one or more RNAs, and the one or more RNAs, lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1. In some embodiments, the N:P ratio is about 5.67:1. In some embodiments, the N:P ratio is about 4.9:1.

### Physical Properties

The characteristics of a lipid nanoparticle (e.g., an empty LNP or a loaded LNP) may depend on the components thereof. For example, a lipid nanoparticle (e.g., an empty LNP or a loaded LNP) including cholesterol as a structural lipid may have different characteristics than a lipid nanoparticle (e.g., an empty LNP or a loaded LNP) that includes a different structural lipid. Similarly, the characteristics of a lipid nanoparticle (e.g., an empty LNP or a loaded LNP) may depend on the absolute or relative amounts of its components. For instance, a lipid nanoparticle (e.g., an empty LNP or a loaded LNP) including a higher molar fraction of a phospholipid may have different characteristics than a lipid nanoparticle (e.g., an empty LNP or a loaded LNP) including a lower molar fraction of a phospholipid. Characteristics may also vary depending on the method and conditions of preparation of the nanoparticle composition.

Lipid nanoparticles (e.g., empty LNPs or loaded LNPs) may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure zeta potentials. Dynamic light scattering may also be utilized to determine particle sizes. Zeta potential can be measured on a Wyatt Technologies Mobius Zeta Potential instrument. This instrument characterizes the mobility and zeta potential by the principle of “Massively Parallel Phase Analysis Light Scattering” or MP-PALS. Without wishing to be bound by theory, this measurement is more sensitive and less stress inducing than ISO Method 13099-1:2012 which only uses one angle of detection and required higher voltage for operation. In some embodiments, the zeta potential of the herein described empty LNP compositions lipid is measured using an instrument employing the principle of MP-PALS. Zeta potential can be measured on a Malvern Zetasizer (Nano ZS).

In some embodiments, the mean diameter of a lipid nanoparticle of the disclosure (e.g., an empty LNP or a loaded LNP) is between 10 s of nm and 100 s of nm as measured by dynamic light scattering (DLS). For example, in some embodiments, the mean diameter of a lipid nanoparticle of the disclosure is from about 40 nm to about 150 nm. In some embodiments, the mean diameter of a lipid nanoparticle of the disclosure is about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean diameter of a lipid nanoparticle (e.g., an empty LNP or a loaded LNP) is from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 150 nm, from about 70 nm to about 130 nm, from about 70 nm to about 100 nm, from about 70 nm to about 90 nm, from about 70 nm to about 80 nm, from about 80 nm to about 150 nm, from about 80 nm to about 130 nm, from about 80 nm to about 100 nm, from about 80 nm to about 90 nm, from about 90 nm to about 150 nm, from about 90 nm to about 130 nm, or from about 90 nm to about 100 nm. In certain embodiments, the mean diameter of a lipid nanoparticle (e.g., an empty LNP or a loaded LNP) of the disclosure is from about 70 nm to about 130 nm or from about 70 nm to about 100 nm. In some embodiments, the mean diameter of a nanoparticle of the disclosure is about 80 nm. In some embodiments, the mean diameter of a nanoparticle of the disclosure is about 100 nm. In some embodiments, the mean diameter of a nanoparticle of the disclosure is about 110 nm. In some embodiments, the mean diameter of a nanoparticle of the disclosure is about 120 nm.

In some embodiments, the polydispersity index (“PDI”) of a plurality of lipid nanoparticles (e.g., empty LNPs or loaded LNPs) formulated with lipids of the disclosure is less than 0.3. In some embodiments, plurality of lipid nanoparticles (e.g., empty LNPs or loaded LNPs) formulated with lipids of the disclosure has a polydispersity index of from about 0 to about 0.25. In some embodiments, plurality of lipid nanoparticles (e.g., empty LNPs or loaded LNPs) formulated with lipids of the disclosure has a polydispersity index of from about 0.10 to about 0.20.

Surface hydrophobicity of nanoparticles of the disclosure can be measured by Generalized Polarization by Laurdan (GPL). In this method, Laurdan, a fluorescent aminonaphthalene ketone lipid, is post-inserted into the nanoparticle surface and the fluorescence spectrum of Laurdan is collected to determine the normalized Generalized Polarization (N-GP). In some embodiments, nanoparticles formulated with lipids of the disclosure have a surface hydrophobicity expressed as N-GP of between about 0.5 and about 1.5. For example, in some embodiments, nanoparticles formulated with lipids of the disclosure have a surface hydrophobicity expressed as N-GP of about 0.5, about 0.6, about 0.7, about 0.8, about 0.9, about 1.0, about 1.1, about 1.2, about 1.3, about 1.4, or about 1.5. In some embodiments, nanoparticles formulated with lipids of the disclosure have a surface hydrophobicity expressed as N-GP of about 1.0 or about 1.1.

The zeta potential of a lipid nanoparticle (e.g., an empty LNP or a loaded LNP) may be used to indicate the electrokinetic potential of the composition. For example, the zeta potential may describe the surface charge of colloidal dispersions, e.g., a nanoparticle composition. Lipid nanoparticles (e.g., empty LNPs or loaded LNPs) with relatively low charges, positive or negative, are generally desirable, as more highly charged species may interact undesirably with cells, tissues, and other elements in the body. The magnitude of the zeta potential indicates the degree of electrostatic repulsion between adjacent, similarly charged particles in the dispersion. In some embodiments, the zeta potential of a lipid nanoparticle (e.g., an empty LNP or a loaded LNP) may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic describes the amount of therapeutic and/or prophylactic that is encapsulated or otherwise associated with a lipid nanoparticle (e.g., an empty LNP or a loaded LNP) after preparation, relative to the initial amount provided. The encapsulation efficiency is desired to be high (e.g., close to 100%). The encapsulation efficiency may be measured, for example, by comparing the amount of therapeutic and/or prophylactic in a solution containing a loaded LNP before and after breaking up the loaded LNP with one or more organic solvents or detergents. Fluorescence may be used to measure the amount of free therapeutic and/or prophylactic (e.g., RNA) in a solution. For the loaded LNPs formulated with lipids of the disclosure, the encapsulation efficiency of a therapeutic and/or prophylactic is at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency is at least 80%. In some embodiments, the encapsulation efficiency is at least 90%. In some embodiments, the encapsulation efficiency of the therapeutic and/or prophylactic agent is between 80% and 100%.

### Pharmaceutical Compositions

Lipid nanoparticles (e.g., empty LNPs or loaded LNPs) may be formulated in whole or in part as pharmaceutical compositions. Pharmaceutical compositions may include one or more lipid nanoparticles (e.g., empty LNPs or loaded LNPs). In one embodiment, a pharmaceutical composition comprises a population of lipid nanoparticles (e.g., empty LNPs or loaded LNPs). For example, a pharmaceutical composition may include one or more lipid nanoparticles (e.g., empty LNPs or loaded LNPs) including one or more different therapeutic and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a nanoparticle composition. An excipient or accessory ingredient may be incompatible with a component of a lipid nanoparticle (e.g., an empty LNP or a loaded LNP) if its combination with the component may result in any undesirable biological effect or otherwise deleterious effect.

In some embodiments, one or more excipients or accessory ingredients may make up greater than 50% of the total mass or volume of a pharmaceutical composition including a nanoparticle composition. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more lipid nanoparticles (e.g., empty LNPs or loaded LNPs), the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more lipid nanoparticles (e.g., empty LNPs or loaded LNPs).

In certain embodiments, the lipid nanoparticles (e.g., empty LNPs or loaded LNPs) and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising a cationic lipid of Formula (I) is a solution that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles (e.g., empty LNPs or loaded LNPs) and/or pharmaceutical compositions comprising a cationic lipid of any of Formula (I) by storing the lipid nanoparticles (e.g., empty LNPs or loaded LNPs) and/or pharmaceutical compositions at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.). For example, the lipid nanoparticles (e.g., empty LNPs or loaded LNPs) and/or pharmaceutical compositions disclosed herein are stable for about at least 1 week, at least 2 weeks, at least 3 weeks, at least 4 weeks, at least 5 weeks, at least 6 weeks, at least 1 month, at least 2 months, at least 4 months, at least 6 months, at least 8 months, at least 10 months, at least 12 months, at least 14 months, at least 16 months, at least 18 months, at least 20 months, at least 22 months, or at least 24 months, e.g., at a temperature of 4° C. or lower (e.g., between about 4° C. and −20° C.). In some embodiments, the formulation is stabilized for at least 4 weeks at about 4° C. In certain embodiments, the pharmaceutical composition of the disclosure comprises a lipid nanoparticle (e.g., an empty LNP or a loaded LNP) disclosed herein and a pharmaceutically acceptable carrier selected from one or more of Tris, an acetate (e.g., sodium acetate), an citrate (e.g., sodium citrate), saline, PBS, and sucrose. In certain embodiments, the pharmaceutical composition of the disclosure has a pH value between about 7 and 8 (e.g., 6.8 6.9, 7.0, 7.1, 7.2, 7.3, 7.4, 7.5, 7.6, 7.7, 7.8, 7.9 or 8.0, or between 7.5 and 8 or between 7 and 7.8). For example, a pharmaceutical composition of the disclosure comprises a lipid nanoparticle (e.g., an empty LNP or a loaded LNP) disclosed herein, Tris, saline and sucrose, and has a pH of about 7.5-8, which is suitable for storage and/or shipment at, for example, about −20° C. For example, a pharmaceutical composition of the disclosure comprises a lipid nanoparticle (e.g., an empty LNP or a loaded LNP) disclosed herein and PBS and has a pH of about 7-7.8, suitable for storage and/or shipment at, for example, about 4° C. or lower. “Stability,” “stabilized,” and “stable” in the context of the present disclosure refers to the resistance of lipid nanoparticles (e.g., empty LNPs or loaded LNPs) and/or pharmaceutical compositions disclosed herein to chemical or physical changes (e.g., degradation, particle size change, aggregation, change in encapsulation, etc.) under given manufacturing, preparation, transportation, storage and/or in-use conditions, e.g., when stress is applied such as shear force, freeze/thaw stress, etc.

In some embodiments, a pharmaceutical composition of the disclosure comprises a empty LNP or a loaded LNP, a cryoprotectant, a buffer, or a combination thereof.

In some embodiments, the cryoprotectant comprises one or more cryoprotective agents, and each of the one or more cryoprotective agents is independently a polyol (e.g., a diol or a triol such as propylene glycol (i.e., 1,2-propanediol), 1,3-propanediol, glycerol, (+/−)-2-methyl-2,4-pentanediol, 1,6-hexanediol, 1,2-butanediol, 2,3-butanediol, ethylene glycol, or diethylene glycol), a nondetergent sulfobetaine (e.g., NDSB-201 (3-(1-pyridino)-1-propane sulfonate), an osmolyte (e.g., L-proline or trimethylamine N-oxide dihydrate), a polymer (e.g., polyethylene glycol 200 (PEG 200), PEG 400, PEG 600, PEG 1000, PEG₂ₖ-DMG, PEG 3350, PEG 4000, PEG 8000, PEG 10000, PEG 20000, polyethylene glycol monomethyl ether 550 (mPEG 550), mPEG 600, mPEG 2000, mPEG 3350, mPEG 4000, mPEG 5000, polyvinylpyrrolidone (e.g., polyvinylpyrrolidone K 15), pentaerythritol propoxylate, or polypropylene glycol P 400), an organic solvent (e.g., dimethyl sulfoxide (DMSO) or ethanol), a sugar (e.g., D-(+)-sucrose, D-sorbitol, trehalose, D-(+)-maltose monohydrate, meso-erythritol, xylitol, myo-inositol, D-(+)-raffinose pentahydrate, D-(+)-trehalose dihydrate, or D-(+)-glucose monohydrate), or a salt (e.g., lithium acetate, lithium chloride, lithium formate, lithium nitrate, lithium sulfate, magnesium acetate, sodium acetate, sodium chloride, sodium formate, sodium malonate, sodium nitrate, sodium sulfate, or any hydrate thereof), or any combination thereof. In some embodiments, the cryoprotectant comprises sucrose. In some embodiments, the cryoprotectant and/or excipient is sucrose. In some embodiments, the cryoprotectant comprises sodium acetate. In some embodiments, the cryoprotectant and/or excipient is sodium acetate. In some embodiments, the cryoprotectant comprises sucrose and sodium acetate.

In some embodiments, wherein the buffer is selected from the group consisting of an acetate buffer, a citrate buffer, a phosphate buffer, a tris buffer, and combinations thereof.

Lipid nanoparticles (e.g., empty LNPs or loaded LNPs) and/or pharmaceutical compositions including one or more lipid nanoparticles (e.g., empty LNPs or loaded LNPs) may be administered to any patient or subject, including those patients or subjects that may benefit from a therapeutic effect provided by the delivery of a therapeutic and/or prophylactic to one or more particular cells, tissues, organs, or systems or groups thereof. Although the descriptions provided herein of lipid nanoparticles (e.g., empty LNPs or loaded LNPs) and pharmaceutical compositions including lipid nanoparticles (e.g., empty LNPs or loaded LNPs) are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats. The subject lipid nanoparticles can also be employed for in vitro and ex vivo uses.

A pharmaceutical composition including one or more lipid nanoparticles (e.g., empty LNPs or loaded LNPs) may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., nanoparticle composition). The amount of the active ingredient is generally equal to the dosage of the active ingredient which would be administered to a subject and/or a convenient fraction of such a dosage such as, for example, one-half or one-third of such a dosage.

Pharmaceutical compositions may be prepared in a variety of forms suitable for a variety of routes and methods of administration. For example, pharmaceutical compositions may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutic and/or prophylactics, additional agents such as wetting agents, emulsifying and suspending agents, sweetening, flavoring, and/or perfuming agents. In certain embodiments for parenteral administration, compositions are mixed with solubilizing agents such as Cremophor*, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, films, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g., carboxymethylcellulose, alginates, gelatin, polyvinylpyrrolidinone, sucrose, and acacia), humectants (e.g., glycerol), disintegrating agents (e.g., agar, calcium carbonate, potato or tapioca starch, alginic acid, certain silicates, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay, silicates), and lubricants (e.g., talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants, and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices. Intradermal compositions may be administered by devices which limit the effective penetration length of a needle into the skin. Jet injection devices which deliver liquid compositions to the dermis via a liquid jet injector and/or via a needle which pierces the stratum corneum and produces a jet which reaches the dermis are suitable. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (wt/wt) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (wt/wt) of the composition, and active ingredient may constitute 0.1% to 20% (wt/wt) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (wt/wt) and as much as 100% (wt/wt) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (wt/wt) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (wt/wt) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this present disclosure.

mRNA Therapies

mRNA as a drug modality has the potential to deliver secreted proteins as well as intracellular proteins and transmembrane proteins. mRNA as a drug modality has the potential to deliver transmembrane and intracellular proteins, i.e., targets that standard biologics are unable to access owing to their inability to cross the cell membrane when delivered in protein form. One major challenge to making mRNA based therapies a reality is the identification of an optimal delivery vehicle. Due to its large size, chemical instability and potential immunogenicity, mRNA requires a delivery vehicle that can offer protection from endo- and exo-nucleases, as well as shield the cargo from immune sentinels. Lipid nanoparticles (LNPs) have been identified as a leading option in this regard.

Key performance criteria for a lipid nanoparticle delivery system are to maximize cellular uptake and enable efficient release of mRNA from the endosome. In one embodiment, the subject LNPs comprising the novel lipids disclosed herein, demonstrate improvements in at least one of cellular uptake and endosomal release. At the same time the LNP must provide a stable drug product and be able to be dosed safely at therapeutically relevant levels. LNPs are multi-component systems which typically consist of an amino lipid, phospholipid, cholesterol, and a PEG-lipid. Each component is required for aspects of efficient delivery of the nucleic acid cargo and stability of the particle. The key component thought to drive cellular uptake, endosomal escape, and tolerability is the amino lipid. Cholesterol and the PEG-lipid contribute to the stability of the drug product both in vivo and on the shelf, while the phospholipid provides additional fusogenicity to the LNP, thus helping to drive endosomal escape and rendering the nucleic acid bioavailable in the cytosol of cells.

Several amino lipid series have been developed for oligonucleotide delivery over the past couple of decades, including the amino lipid MC3 (DLin-MC3-DMA). MC3-based LNPs have been shown to be effective in delivering mRNA. LNPs of this class are quickly opsonized by apolipoprotein E (ApoE) when delivered intravenously, which enables cellular uptake by the low density lipoprotein receptor (LDLr). However, concerns remain that MC3's long tissue half-life could contribute to unfavorable side effects hindering its use for chronic therapies. In addition, extensive literature evidence suggests that chronic dosing of lipid nanoparticles can produce several toxic sides effects including complement activation-related pseudo allergy (CARPA) and liver damage. Hence, to unleash the potential of mRNA and other nucleic acid, nucleoptide or peptide based therapies for humans, a class of LNPs with increased delivery efficiency along with a metabolic and toxicity profile that would enable chronic dosing in humans is needed.

The ability to treat a broad swath of diseases requires the flexibility to safely dose chronically at varying dose levels. Through systematic optimization of the amino lipid structure, the lipids of the disclosure were identified as lipids that balance chemical stability, improved efficiency of delivery due to improved endosomal escape, rapid in vivo metabolism, and a clean toxicity profile. The combination of these features provides a drug candidate that can be dosed chronically without activation of the immune system. Initial rodent screens led to the identification of a lead lipid with good delivery efficiency and pharmacokinetics. The lead LNP was profiled further in non-human primate for efficiency of delivery after single and repeat dosing. Finally, the optimized LNPs were evaluated in one-month repeat dose toxicity studies in rat and non-human primate. Without wishing to be bound by theory, the novel ionizable lipids of the instant disclosure have the improved cellular delivery, improved protein expression, and improved biodegradability properties that can lead to greater than 2 fold, 5 fold, 10 fold, 15 fold, or 20 fold increase in mRNA expression in cells as compared to LNPs which lack a lipid of the invention. In another embodiment, an LNP comprising a lipid of the invention can result in specific (e.g., preferential) delivery to a certain cell type or types as compared other cell types, thereby resulting in a greater than 2 fold, 5 fold, 10 fold, 15 fold, or 20 fold increase in mRNA expression in certain cells or tissues as compared to LNPs which lack a lipid of the invention. These improvements over the art allow for the safe and effective use of mRNA-based therapies in acute and chronic diseases.

### Methods of Use

In some aspects, the disclosure provides a method of delivering a therapeutic and/or prophylactic to a cell (e.g., a mammalian cell). This method includes the step of contacting the cell with a loaded LNP or a pharmaceutical composition of the disclosure, whereby the therapeutic and/or prophylactic is delivered to the cell. In some embodiments, the cell is in a subject and the contacting comprises administering the cell to the subject. In some embodiments, the method comprises the step of administering to the subject a lipid nanoparticle comprising a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), a phospholipid, a structural lipid, a PEG lipid, and one or more therapeutic and/or prophylactic agents, whereby the therapeutic and/or prophylactic is delivered to the cell.

In some embodiments, the disclosure provides a method of delivering a therapeutic and/or prophylactic to a cell within a subject, wherein the method comprises the step of administering to the subject a lipid nanoparticle comprising a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), DSPC, cholesterol, and PEG₂ₖ-DMG, and one or more therapeutic and/or prophylactic agents selected from a nucleotide, a polypeptide, and a nucleic acid (e.g., an RNA).

In some embodiments, the disclosure provides a method of delivering a therapeutic and/or prophylactic to a cell within a subject, wherein the method comprises the step of administering to the subject a lipid nanoparticle comprising a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), DSPC, cholesterol, and PEG-1, and one or more therapeutic and/or prophylactic agents selected from a nucleotide, a polypeptide, and a nucleic acid (e.g., an RNA).

In some aspects, the disclosure provides a method of delivering (e.g., specifically delivering) a therapeutic and/or prophylactic to a mammalian organ or tissue (e.g., a liver, kidney, spleen, or lung). This method includes the step of contacting the cell with a loaded LNP or a pharmaceutical composition of the disclosure, whereby the therapeutic and/or prophylactic is delivered to the target organ or tissue. In some embodiments, the method comprises the step of administering to the subject a lipid nanoparticle comprising a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), a phospholipid, a structural lipid, a PEG lipid, and one or more therapeutic and/or prophylactic agents, whereby the therapeutic and/or prophylactic is delivered to the target organ or tissue. In some embodiments, the target organ is the lung or the target tissue is tissue is the pulmonary endothelium.

In some embodiments, the disclosure provides a method of specifically delivering a therapeutic and/or prophylactic to an organ of a subject, wherein the method comprises the step of administering to the subject a lipid nanoparticle comprising a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), DSPC, cholesterol, and PEG₂ₖ-DMG, and one or more therapeutic and/or prophylactic agents selected from a nucleotide, a polypeptide, and a nucleic acid (e.g., an RNA).

In some embodiments, the disclosure provides a method of specifically delivering a therapeutic and/or prophylactic to an organ of a subject, wherein the method comprises the step of administering to the subject a lipid nanoparticle comprising a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), DSPC, cholesterol, and PEG-1, and one or more therapeutic and/or prophylactic agents selected from a nucleotide, a polypeptide, and a nucleic acid (e.g., an RNA).

In some aspects, the disclosure features a method for the enhanced delivery of a therapeutic and/or prophylactic (e.g., an mRNA) to a target tissue (e.g., a liver, spleen, or lung). This method includes the step of contacting the cell with a loaded LNP or a pharmaceutical composition of the disclosure, whereby the therapeutic and/or prophylactic is delivered to the target tissue (e.g., a liver, kidney, spleen, or lung). In some embodiments, the target tissue is the pulmonary endothelium. In some embodiments, the method comprises the step of administering to the subject a lipid nanoparticle comprising a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), a phospholipid, a structural lipid, a PEG lipid, and one or more therapeutic and/or prophylactic agents, whereby the therapeutic and/or prophylactic is delivered to the target tissue (e.g., a liver, kidney, spleen, or lung). In some embodiments, the target tissue is the pulmonary endothelium.

In some embodiments, the disclosure provides a method for the enhanced delivery of a therapeutic and/or prophylactic to a target tissue, wherein the method comprises the step of administering to the subject a lipid nanoparticle comprising a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), DSPC, cholesterol, and PEG₂ₖ-DMG, and one or more therapeutic and/or prophylactic agents selected from a nucleotide, a polypeptide, and a nucleic acid (e.g., an RNA).

In some embodiments, the disclosure provides a method for the enhanced delivery of a therapeutic and/or prophylactic to a target tissue, wherein the method comprises the step of administering to the subject a lipid nanoparticle comprising a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), DSPC, cholesterol, and PEG-1, and one or more therapeutic and/or prophylactic agents selected from a nucleotide, a polypeptide, and a nucleic acid (e.g., an RNA). In some embodiments, the target tissue is the pulmonary endothelium.

In some aspects, the disclosure provides a method of producing a polypeptide of interest in a cell (e.g., a mammalian cell). This method includes the step of contacting the cell with a loaded LNP or a pharmaceutical composition of the disclosure, wherein the loaded LNP or pharmaceutical composition comprises an mRNA, whereby the mRNA is capable of being translated in the cell to produce the polypeptide. In some embodiments, the cell is in a subject and the contacting comprises administering the cell to the subject. In some embodiments, the method comprises the step of administering to the subject a lipid nanoparticle comprising a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), a phospholipid, a structural lipid, a PEG lipid, and an mRNA, whereby the mRNA is capable of being translated in the cell to produce the polypeptide.

In some embodiments, the disclosure provides a method of producing a polypeptide of interest in a cell, wherein the method comprises the step of administering to the subject a lipid nanoparticle comprising a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), DSPC, cholesterol, and PEG₂ₖ-DMG, and an mRNA. For example, in some embodiments, the disclosure provides a method of producing a polypeptide of interest in a cell, wherein the method comprises the step of administering to the subject a lipid nanoparticle comprising a cationic lipid of Table 1, an ionizable lipid of Tables IL-1 to IL-7, DSPC, cholesterol, and PEG₂ₖ-DMG, and an mRNA.

In some embodiments, the disclosure provides a method of producing a polypeptide of interest in a cell, wherein the method comprises the step of administering to the subject a lipid nanoparticle comprising a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), DSPC, cholesterol, and PEG-1, and an mRNA. For example, in some embodiments, the disclosure provides a method of producing a polypeptide of interest in a cell, wherein the method comprises the step of administering to the subject a lipid nanoparticle comprising a cationic lipid of Table 1, an ionizable lipid of Tables IL-1 to IL-7, DSPC, cholesterol, and PEG-1, and an mRNA.

In some aspects, the disclosure provides a method of treating a disease or disorder in a mammal (e.g., a human) in need thereof. The method includes the step of administering to the mammal a therapeutically effective amount of loaded LNP or a pharmaceutical composition of the disclosure. In some embodiments, the method comprises the step of administering to the subject a lipid nanoparticle comprising a cationic lipid of Formula (I), an ionizable lipid of Formula (TL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), a phospholipid, a structural lipid, a PEG lipid, and one or more therapeutic and/or prophylactic agents, whereby the therapeutic and/or prophylactic is delivered to the cell. In some embodiments, the disease or disorder is characterized by dysfunctional or aberrant protein or polypeptide activity. For example, the disease or disorder is selected from the group consisting of rare diseases, infectious diseases, cancer and proliferative diseases, genetic diseases, autoimmune diseases, diabetes, neurodegenerative diseases, cardio- and reno-vascular diseases, and metabolic diseases.

In some embodiments, the disclosure provides a method of treating a disease or disorder in a subject, wherein the method comprises the step of administering to the subject a lipid nanoparticle comprising a cationic lipid of Formula (I), an ionizable lipid of Formula (IL-A), (TL-B), (TL-C), (TL-D), (IL-I), (IL-IA), (IL-IB), (IL-IC), (IL-IIA), (IL-IIAX), (IL-IIB), (IL-IIC), (IL-III), or (IL-IIIA), DSPC, cholesterol, and PEG₂ₖ-DMG, and one or more therapeutic and/or prophylactic agents selected from a nucleotide, a polypeptide, and a nucleic acid (e.g., an RNA). For example, in some embodiments, the disclosure provides a method of treating a disease or disorder in a subject, wherein the method comprises the step of administering to the subject a lipid nanoparticle comprising a cationic lipid of Table 1, an ionizable lipid of Tables IL-1 to IL-7, DSPC, cholesterol, and PEG₂ₖ-DMG, and one or more therapeutic and/or prophylactic agents selected from a nucleotide, a polypeptide, and a nucleic acid (e.g., an RNA).

In some embodiments, the disclosure provides a method of treating a disease or disorder in a subject, wherein the method comprises the step of administering to the subject a lipid nanoparticle comprising a cationic lipid of Formula (I), an ionizable lipid of Formula (IL-A), (TL-B), (TL-C), (TL-D), (IL-I), (IL-IA), (IL-IB), (IL-IC), (IL-IIA), (IL-IIAX), (IL-IIB), (IL-IIC), (IL-III), or (IL-IIIA), DSPC, cholesterol, and PEG-1, and one or more therapeutic and/or prophylactic agents selected from a nucleotide, a polypeptide, and a nucleic acid (e.g., an RNA). For example, in some embodiments, the disclosure provides a method of treating a disease or disorder in a subject, wherein the method comprises the step of administering to the subject a lipid nanoparticle comprising a cationic lipid of Table 1, an ionizable lipid of Tables IL-1 to IL-7, DSPC, cholesterol, and PEG-1, and one or more therapeutic and/or prophylactic agents selected from a nucleotide, a polypeptide, and a nucleic acid (e.g., an RNA).

In yet another aspect, the disclosure features a method of lowering immunogenicity comprising introducing loaded LNP or a pharmaceutical composition of the disclosure into cells, wherein the loaded LNP or a pharmaceutical composition reduces the induction of the cellular immune response of the cells to the loaded LNP or a pharmaceutical composition, as compared to the induction of the cellular immune response in cells induced by a reference composition. In some embodiments, the cell is in a subject and the contacting comprises administering the cell to the subject. In some embodiments, the method comprises the step of administering to the subject a lipid nanoparticle comprising a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), a phospholipid, a structural lipid, a PEG lipid, and one or more therapeutic and/or prophylactic agents selected from a nucleotide, a polypeptide, and a nucleic acid (e.g., an RNA), wherein the lipid nanoparticle comprising a cationic lipid of Formula (I), and an ionizable lipid of Formula (IL-A), (TL-B), (TL-C), (TL-D), (IL-I), (IL-IA), (IL-IB), (IL-IC), (IL-IIA), (IL-IIAX), (IL-IIB), (IL-IIC), (IL-III), or (IL-IIIA) reduces the induction of the cellular immune response of the cells to the lipid nanoparticle comprising a cationic lipid of Formula (I), and an ionizable lipid of Formula (TL-A), (TL-B), (TL-C), (IL-D), (IL-I), (IL-IA), (IL-IB), (IL-IC), (IL-IIA), (IL-IIAX), (IL-IIB), (IL-IIC), (IL-III), or (IL-IIIA), as compared to the induction of the cellular immune response in cells induced by a reference composition. For example, the cellular immune response is an innate immune response, an adaptive immune response, or both.

In some embodiments, the disclosure provides a method of lowering immunogenicity in a subject, wherein the method comprises the step of administering to the subject a lipid nanoparticle comprising a cationic lipid of Formula (I), an ionizable lipid of Formula (IL-A), (IL-B), (TL-C), (TL-D), (IL-I), (IL-IA), (IL-IB), (IL-IC), (IL-IIA), (IL-IIAX), (IL-IIB), (IL-IIC), (IL-III), or (IL-IIIA), DSPC, cholesterol, and PEG₂ₖ-DMG, and one or more therapeutic and/or prophylactic agents selected from a nucleotide, a polypeptide, and a nucleic acid (e.g., an RNA). For example, in some embodiments, the disclosure provides a method of lowering immunogenicity in a subject, wherein the method comprises the step of administering to the subject a lipid nanoparticle comprising a cationic lipid of Table 1, an ionizable lipid of Tables IL-1 to IL-7, DSPC, cholesterol, and PEG₂ₖ-DMG, and one or more therapeutic and/or prophylactic agents selected from a nucleotide, a polypeptide, and a nucleic acid (e.g., an RNA).

In some embodiments, the disclosure provides a method of lowering immunogenicity in a subject, wherein the method comprises the step of administering to the subject a lipid nanoparticle comprising a cationic lipid of Formula (I), an ionizable lipid of Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), DSPC, cholesterol, and PEG-1, and one or more therapeutic and/or prophylactic agents selected from a nucleotide, a polypeptide, and a nucleic acid (e.g., an RNA). For example, in some embodiments, the disclosure provides a method of lowering immunogenicity in a subject, wherein the method comprises the step of administering to the subject a lipid nanoparticle comprising a cationic lipid of Table 1, an ionizable lipid of Tables IL-1 to IL-7, DSPC, cholesterol, and PEG-1, and one or more therapeutic and/or prophylactic agents selected from a nucleotide, a polypeptide, and a nucleic acid (e.g., an RNA).

The disclosure also includes methods of synthesizing a cationic lipid of Formula (I), and methods of making a lipid nanoparticle (e.g., an empty LNP or a loaded LNP) including a lipid component comprising the cationic lipid of Formula (I).

### Methods of Producing Polypeptides in Cells

The present disclosure provides methods of producing a polypeptide of interest in a mammalian cell. Methods of producing polypeptides involve contacting a cell with a lipid nanoparticle (e.g., an empty LNP or a loaded LNP) including an mRNA encoding the polypeptide of interest. Upon contacting the cell with the nanoparticle composition, the mRNA may be taken up and translated in the cell to produce the polypeptide of interest.

In general, the step of contacting a mammalian cell with a lipid nanoparticle (e.g., an empty LNP or a loaded LNP) including an mRNA encoding a polypeptide of interest may be performed in vivo, ex vivo, in culture, or in vitro. The amount of lipid nanoparticle (e.g., an empty LNP or a loaded LNP) contacted with a cell, and/or the amount of mRNA therein, may depend on the type of cell or tissue being contacted, the means of administration, the physiochemical characteristics of the lipid nanoparticle (e.g., an empty LNP or a loaded LNP) and the mRNA (e.g., size, charge, and chemical composition) therein, and other factors. In general, an effective amount of the lipid nanoparticle (e.g., an empty LNP or a loaded LNP) will allow for efficient polypeptide production in the cell. Metrics for efficiency may include polypeptide translation (indicated by polypeptide expression), level of mRNA degradation, and immune response indicators.

The step of contacting a lipid nanoparticle (e.g., an empty LNP or a loaded LNP) including an mRNA with a cell may involve or cause transfection. A phospholipid including in the lipid component of a lipid nanoparticle (e.g., an empty LNP or a loaded LNP) may facilitate transfection and/or increase transfection efficiency, for example, by interacting and/or fusing with a cellular or intracellular membrane. Transfection may allow for the translation of the mRNA within the cell.

In some embodiments, the lipid nanoparticles (e.g., empty LNPs or loaded LNPs) described herein may be used therapeutically. For example, an mRNA included in a lipid nanoparticle (e.g., an empty LNP or a loaded LNP) may encode a therapeutic polypeptide (e.g., in a translatable region) and produce the therapeutic polypeptide upon contacting and/or entry (e.g., transfection) into a cell. In other embodiments, an mRNA included in a lipid nanoparticle (e.g., an empty LNP or a loaded LNP) may encode a polypeptide that may improve or increase the immunity of a subject. For example, an mRNA may encode a granulocyte-colony stimulating factor or trastuzumab.

In certain embodiments, an mRNA included in a lipid nanoparticle (e.g., an empty LNP or a loaded LNP) may encode a recombinant polypeptide that may replace one or more polypeptides that may be substantially absent in a cell contacted with the nanoparticle composition. The one or more substantially absent polypeptides may be lacking due to a genetic mutation of the encoding gene or a regulatory pathway thereof. Alternatively, a recombinant polypeptide produced by translation of the mRNA may antagonize the activity of an endogenous protein present in, on the surface of, or secreted from the cell. An antagonistic recombinant polypeptide may be desirable to combat deleterious effects caused by activities of the endogenous protein, such as altered activities or localization caused by mutation. In another alternative, a recombinant polypeptide produced by translation of the mRNA may indirectly or directly antagonize the activity of a biological moiety present in, on the surface of, or secreted from the cell. Antagonized biological moieties may include, but are not limited to, lipids (e.g., cholesterol), lipoproteins (e.g., low density lipoprotein), nucleic acids, carbohydrates, and small molecule toxins. Recombinant polypeptides produced by translation of the mRNA may be engineered for localization within the cell, such as within a specific compartment such as the nucleus, or may be engineered for secretion from the cell or for translocation to the plasma membrane of the cell.

In some embodiments, contacting a cell with a lipid nanoparticle (e.g., an empty LNP or a loaded LNP) including an mRNA may reduce the innate immune response of a cell to an exogenous nucleic acid. A cell may be contacted with a first lipid nanoparticle (e.g., an empty LNP or a loaded LNP) including a first amount of a first exogenous mRNA including a translatable region and the level of the innate immune response of the cell to the first exogenous mRNA may be determined. Subsequently, the cell may be contacted with a second composition including a second amount of the first exogenous mRNA, the second amount being a lesser amount of the first exogenous mRNA compared to the first amount. Alternatively, the second composition may include a first amount of a second exogenous mRNA that is different from the first exogenous mRNA. The steps of contacting the cell with the first and second compositions may be repeated one or more times. Additionally, efficiency of polypeptide production (e.g., translation) in the cell may be optionally determined, and the cell may be re-contacted with the first and/or second composition repeatedly until a target protein production efficiency is achieved.

### Methods of Delivering Therapeutic Agents to Cells and Organs

The present disclosure provides methods of delivering a therapeutic and/or prophylactic to a mammalian cell or organ. Delivery of a therapeutic and/or prophylactic to a cell involves administering a lipid nanoparticle (e.g., an empty LNP or a loaded LNP) including the therapeutic and/or prophylactic to a subject, where administration of the composition involves contacting the cell with the composition. For example, a protein, cytotoxic agent, radioactive ion, chemotherapeutic agent, or nucleic acid (such as an RNA, e.g., mRNA) may be delivered to a cell or organ. In the instance that a therapeutic and/or prophylactic is an mRNA, upon contacting a cell with the nanoparticle composition, a translatable mRNA may be translated in the cell to produce a polypeptide of interest. However, mRNAs that are substantially not translatable may also be delivered to cells. Substantially non-translatable mRNAs may be useful as vaccines and/or may sequester translational components of a cell to reduce expression of other species in the cell.

In some embodiments, a lipid nanoparticle (e.g., an empty LNP or a loaded LNP) may target a particular type or class of cells (e.g., cells of a particular organ or system thereof). For example, a lipid nanoparticle (e.g., an empty LNP or a loaded LNP) including a therapeutic and/or prophylactic of interest may be specifically delivered to a mammalian liver, kidney, spleen, or lung. Specific delivery to a particular class of cells, an organ, or a system or group thereof implies that a higher proportion of lipid nanoparticles (e.g., loaded LNPs) including a therapeutic and/or prophylactic are delivered to the destination (e.g., tissue) of interest relative to other destinations. In some embodiments, specific delivery of a loaded LNP comprising an mRNA may result in a greater than 2 fold, 5 fold, 10 fold, 15 fold, or 20 fold increase in mRNA expression in cells of the targeted destination (e.g., tissue of interest, such as a liver) as compared to cells of another destination (e.g., the spleen). In some embodiments, the tissue of interest is selected from the group consisting of a liver, a kidney, a lung, a spleen, and tumor tissue (e.g., via intratumoral injection).

As another example of targeted or specific delivery, an mRNA that encodes a protein-binding partner (e.g., an antibody or functional fragment thereof, a scaffold protein, or a peptide) or a receptor on a cell surface may be included in a nanoparticle composition. An mRNA may additionally or instead be used to direct the synthesis and extracellular localization of lipids, carbohydrates, or other biological moieties. Alternatively, other therapeutic and/or prophylactics or elements (e.g., lipids or ligands) of a lipid nanoparticle (e.g., an empty LNP or a loaded LNP) may be selected based on their affinity for particular receptors (e.g., low density lipoprotein receptors) such that a lipid nanoparticle (e.g., an empty LNP or a loaded LNP) may more readily interact with a target cell population including the receptors. For example, ligands may include, but are not limited to, members of a specific binding pair, antibodies, monoclonal antibodies, Fv fragments, single chain Fv (scFv) fragments, Fab′ fragments, F(ab′)2 fragments, single domain antibodies, camelized antibodies and fragments thereof, humanized antibodies and fragments thereof, and multivalent versions thereof, multivalent binding reagents including mono- or bi-specific antibodies such as disulfide stabilized Fv fragments, scFv tandems, diabodies, tribodies, or tetrabodies; and aptamers, receptors, and fusion proteins.

In some embodiments, a ligand may be a surface-bound antibody, which can permit tuning of cell targeting specificity. This is especially useful since highly specific antibodies can be raised against an epitope of interest for the desired targeting site. In some embodiments, multiple antibodies are expressed on the surface of a cell, and each antibody can have a different specificity for a desired target. Such approaches can increase the avidity and specificity of targeting interactions.

A ligand can be selected, e.g., by a person skilled in the biological arts, based on the desired localization or function of the cell.

Targeted cells may include, but are not limited to, hepatocytes, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells.

### Methods of Treating Diseases and Disorders

Lipid nanoparticles (e.g., empty LNPs or loaded LNPs) may be useful for treating a disease, disorder, or condition. In particular, such compositions may be useful in treating a disease, disorder, or condition characterized by missing or aberrant protein or polypeptide activity. For example, a lipid nanoparticle (e.g., an empty LNP or a loaded LNP) comprising an mRNA encoding a missing or aberrant polypeptide may be administered or delivered to a cell. Subsequent translation of the mRNA may produce the polypeptide, thereby reducing or eliminating an issue caused by the absence of or aberrant activity caused by the polypeptide. Because translation may occur rapidly, the methods and compositions may be useful in the treatment of acute diseases, disorders, or conditions such as sepsis, stroke, and myocardial infarction. A therapeutic and/or prophylactic included in a lipid nanoparticle (e.g., an empty LNP or a loaded LNP) may also be capable of altering the rate of transcription of a given species, thereby affecting gene expression.

Diseases, disorders, and/or conditions characterized by dysfunctional or aberrant protein or polypeptide activity for which a composition may be administered include, but are not limited to, rare diseases, infectious diseases (as both vaccines and therapeutics), cancer and proliferative diseases, genetic diseases, autoimmune diseases, diabetes, neurodegenerative diseases, cardio- and reno-vascular diseases, and metabolic diseases. Multiple diseases, disorders, and/or conditions may be characterized by missing (or substantially diminished such that proper protein function does not occur) protein activity. Such proteins may not be present, or they may be essentially non-functional. The present disclosure provides a method for treating such diseases, disorders, and/or conditions in a subject by administering a lipid nanoparticle (e.g., an empty LNP or a loaded LNP) including an RNA and a cationic lipid component including a lipid according to Formula (I), an ionizable lipid component including a lipid according to Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), a phospholipid (optionally unsaturated), a PEG lipid, and a structural lipid, wherein the RNA may be an mRNA encoding a polypeptide that antagonizes or otherwise overcomes an aberrant protein activity present in the cell of the subject.

The disclosure provides methods involving administering lipid nanoparticles (e.g., empty LNPs or loaded LNPs) including one or more therapeutic and/or prophylactic agents and pharmaceutical compositions including the same. The terms therapeutic and prophylactic can be used interchangeably herein with respect to features and embodiments of the present disclosure. Therapeutic compositions, or imaging, diagnostic, or prophylactic compositions thereof, may be administered to a subject using any reasonable amount and any route of administration effective for preventing, treating, diagnosing, or imaging a disease, disorder, and/or condition and/or any other purpose. The specific amount administered to a given subject may vary depending on the species, age, and general condition of the subject; the purpose of the administration; the particular composition; the mode of administration; and the like. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment. The specific therapeutically effective, prophylactically effective, or otherwise appropriate dose level (e.g., for imaging) for any particular patient will depend upon a variety of factors including the severity and identify of a disorder being treated, if any; the one or more therapeutic and/or prophylactics employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

A loaded LNP may be administered by any route. In some embodiments, compositions, including prophylactic, diagnostic, or imaging compositions including one or more loaded LNPs described herein, are administered by one or more of a variety of routes, including oral, intravenous, intramuscular, intra-arterial, subcutaneous, trans- or intra-dermal, interdermal, intraperitoneal, mucosal, nasal, intratumoral, intranasal; by inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by any other parenteral route of administration or by inhalation. However, the present disclosure encompasses the delivery or administration of compositions described herein by any appropriate route taking into consideration likely advances in the sciences of drug delivery. In general, the most appropriate route of administration will depend upon a variety of factors including the nature of the loaded LNP including one or more therapeutic and/or prophylactics (e.g., its stability in various bodily environments such as the bloodstream and gastrointestinal tract), the condition of the patient (e.g., whether the patient is able to tolerate particular routes of administration), etc.

In certain embodiments, compositions in accordance with the present disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.05 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, from about 1 mg/kg to about 5 mg/kg, from about 2 mg/kg to about 5 mg/kg, from about 0.0001 mg/kg to about 2.5 mg/kg, from about 0.001 mg/kg to about 2.5 mg/kg, from about 0.005 mg/kg to about 2.5 mg/kg, from about 0.01 mg/kg to about 2.5 mg/kg, from about 0.05 mg/kg to about 2.5 mg/kg, from about 0.1 mg/kg to about 2.5 mg/kg, from about 1 mg/kg to about 2.5 mg/kg, from about 2 mg/kg to about 2.5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, from about 0.05 mg/kg to about 1 mg/kg, from about 0.1 mg/kg to about 1 mg/kg, from about 0.0001 mg/kg to about 0.25 mg/kg, from about 0.001 mg/kg to about 0.25 mg/kg, from about 0.005 mg/kg to about 0.25 mg/kg, from about 0.01 mg/kg to about 0.25 mg/kg, from about 0.05 mg/kg to about 0.25 mg/kg, or from about 0.1 mg/kg to about 0.25 mg/kg of a therapeutic and/or prophylactic (e.g., an mRNA) in a given dose, where a dose of 1 mg/kg (mpk) provides 1 mg of a therapeutic and/or prophylactic per 1 kg of subject body weight. In some embodiments, a dose of about 0.001 mg/kg to about 10 mg/kg of a therapeutic and/or prophylactic of a loaded LNP may be administered. In other embodiments, a dose of about 0.005 mg/kg to about 2.5 mg/kg of a therapeutic and/or prophylactic may be administered. In certain embodiments, a dose of about 0.1 mg/kg to about 1 mg/kg may be administered. In other embodiments, a dose of about 0.05 mg/kg to about 0.25 mg/kg may be administered. A dose may be administered one or more times per day, in the same or a different amount, to obtain a desired level of mRNA expression and/or therapeutic, diagnostic, prophylactic, or imaging effect. The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In some embodiments, a single dose may be administered, for example, prior to or after a surgical procedure or in the instance of an acute disease, disorder, or condition.

Lipid nanoparticles (e.g., empty LNPs or loaded LNPs) including one or more therapeutic and/or prophylactics may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. For example, one or more lipid nanoparticles (e.g., empty LNPs or loaded LNPs) including one or more different therapeutic and/or prophylactics may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects, such as infusion related reactions).

A lipid nanoparticle (e.g., an empty LNP or a loaded LNP) may be used in combination with an agent to increase the effectiveness and/or therapeutic window of the composition. Such an agent may be, for example, an anti-inflammatory compound, a steroid (e.g., a corticosteroid), a statin, an estradiol, a BTK inhibitor, an S1P1 agonist, a glucocorticoid receptor modulator (GRM), or an anti-histamine. In some embodiments, a lipid nanoparticle (e.g., an empty LNP or a loaded LNP) may be used in combination with dexamethasone, methotrexate, acetaminophen, an H1 receptor blocker, or an H2 receptor blocker. In some embodiments, a method of treating a subject in need thereof or of delivering a therapeutic and/or prophylactic to a subject (e.g., a mammal) may involve pre-treating the subject with one or more agents prior to administering a nanoparticle composition. For example, a subject may be pre-treated with a useful amount (e.g., 10 mg, 20 mg, 30 mg, 40 mg, 50 mg, 60 mg, 70 mg, 80 mg, 90 mg, 100 mg, or any other useful amount) of dexamethasone, methotrexate, acetaminophen, an H1 receptor blocker, or an H2 receptor blocker. Pre-treatment may occur 24 or fewer hours (e.g., 24 hours, 20 hours, 16 hours, 12 hours, 8 hours, 4 hours, 2 hours, 1 hour, 50 minutes, 40 minutes, 30 minutes, 20 minutes, or 10 minutes) before administration of the lipid nanoparticle (e.g., an empty LNP or a loaded LNP) and may occur one, two, or more times in, for example, increasing dosage amounts.

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all, of the group members are present in, employed in, or otherwise relevant to a given product or process. As used herein, the expressions “one or more of A, B, or C,” “one or more A, B, or C,” “one or more of A, B, and C,” “one or more A, B, and C”, “selected from A, B, and C,” “selected from the group consisting of A, B, and C,” and the like are used interchangeably and all refer to a selection from a group consisting of A, B, and/or C, i.e., one or more As, one or more Bs, one or more Cs, or any combination thereof, unless otherwise specified.

It is also noted that the term “comprising” is intended to be open and permits but does not require the inclusion of additional elements or steps. When the term “comprising” is used herein, the terms “consisting essentially of” and “consisting of” are thus also encompassed and disclosed. Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

Where ranges are given, endpoints are included. Furthermore, it is to be understood that unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

The synthetic processes of the disclosure can tolerate a wide variety of functional groups, therefore various substituted starting materials can be used. The processes generally provide the desired final lipid at or near the end of the overall process, although it may be desirable in certain instances to further convert the lipid to a pharmaceutically acceptable salt thereof.

Lipids of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3d edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art. The following descriptions of synthetic methods are designed to illustrate, but not to limit, general procedures for the preparation of lipids of the present disclosure.

The cationic lipids of this disclosure having the formula described herein may be prepared according to the procedures illustrated in the scheme below, from commercially available starting materials or starting materials which can be prepared using literature procedures. One of ordinary skill in the art will note that, during the reaction sequences and synthetic schemes described herein, the order of certain steps may be changed.

One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 5^{th} edition, John Wiley & Sons: New York, 2014.

Preferred protecting groups include, but are not limited to:
For a hydroxyl moiety: TBS, benzyl, THP, Ac.
For carboxylic acids: benzyl ester, methyl ester, ethyl ester, allyl ester.
For amines: Fmoc, Cbz, BOC, DMB, Ac, Bn, Tr, Ts, trifluoroacetyl, phthalimide, benzylideneamine.
For diols: Ac (×2) TBS (×2), or when taken together acetonides.
For thiols: Ac.
For benzimidazoles: SEM, benzyl, PMB, DMB.
For aldehydes: di-alkyl acetals such as dimethoxy acetal or diethyl acetyl.

In the reaction schemes described herein, multiple stereoisomers may be produced. When no particular stereoisomer is indicated, it is understood to mean all possible stereoisomers that could be produced from the reaction. A person of ordinary skill in the art will recognize that the reactions can be optimized to give one isomer preferentially, or new schemes may be devised to produce a single isomer. If mixtures are produced, techniques such as preparative thin layer chromatography, preparative HPLC, preparative chiral HPLC, or preparative SFC may be used to separate the isomers.

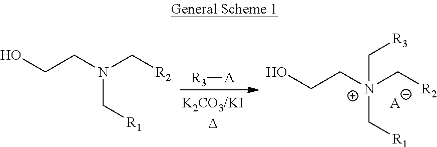

As illustrated in General Scheme 1 above, an ionizable lipid is reacted with a desired tail in base (e.g. K₂CO₃) in the presence of potassium iodide, and is heated to reflux, to provide the cationic lipid of the present disclosure. A⁻ is any pharmaceutically acceptable anion.

A person of ordinary skill in the art will recognize that in the above scheme the order of certain steps may be interchangeable.

In certain aspects, the disclosure also includes methods of synthesizing a cationic lipid of Formula (I) and intermediate(s) for synthesizing the lipid. Without wishing to be bound by theory, it is understood that the anion accompanying the cationic lipid of Formula (I) (i.e., “A⁻”) may be supplied by starting materials involved in the synthesis of the cationic lipid of Formula (I). For example, a Br⁻ ion may be supplied by undecyl 6-bromohexanoate, nonyl 8-bromooctanoate, or 3-butylheptyl 8-bromooctanoate. Without wishing to be bound by theory, identification of the anion may require additional analysis by known methods (e.g., elemental analysis). It is understood, however, that identification of the anion is not a necessary step in the synthesis of the cationic lipid of Formula (I). Without wishing to be bound by theory, during any of the syntheses described herein, the cationic lipid of Formula (I) may be formed with an anion that is replaced with another anion during a purification step. For example, in some embodiments, the cationic lipid of Formula (I) is initially formed with a Br⁻ ion or a Cl⁻ ion, but the Br⁻ ion or Cl⁻ ion is replaced with a OH⁻ ion during a purification step. For example, when NH₄OH is comprised in the eluent for a silica gel column purification, the initial counter ion (e.g., Br⁻ or Cl⁻) may be replaced with a OH⁻ anion. It is understood that, in some embodiments, wherein more than one molecule of a compound of Formula (I) is present, mixtures of anions may be present. For example, without wishing to be bound by theory, if two molecules of a compound of Formula (I) are present, each molecule may have a different counter ion. For example, the couter ion may be Br⁻ of one molecule of the compound of Formula (I) and OH⁻ for the other.

The ionizable lipids described herein may be prepared according to the procedures disclosed in Published International Patent Application Nos. WO/2017/049245, WO/2017/112865, WO/2018/170306, WO/2018/232120, WO/2021/055835, WO/2021/055833, and WO/2021/055849, each of which is incorporated by reference herein in its entirety.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

### Example 1: Synthesis of Cationic Lipids of Table 1

### A. General Considerations

All solvents and reagents used were obtained commercially and used as such unless noted otherwise. ¹H NMR spectra were recorded in CDCl₃, at 300 K using a Bruker Ultrashield 300 MHz instrument. Chemical shifts are reported as parts per million (ppm) relative to TMS (0.00) for ¹H. Silica gel chromatographies were performed on ISCO CombiFlash Rf+ Lumen Instruments using ISCO RediSep Rf Gold Flash Cartridges (particle size: 20-40 microns). Reverse phase chromatographies were performed on ISCO CombiFlash Rf+ Lumen Instruments using RediSep Rf Gold C18 High Performance columns. All final lipids were determined to be greater than 85% pure via analysis by reverse phase UPLC-MS (retention times, RT, in minutes) using Waters Acquity UPLC instrument with DAD and ELSD and a ZORBAX Rapid Resolution High Definition (RRHD) SB-C18 LC column, 2.1 mm, 50 mm, 1.8 μm, and a gradient of 65 to 100% acetonitrile in water with 0.1% TFA over 5 minutes at 1.2 mL/min. Injection volume was 5 μL and the column temperature was 80° C. Detection was based on electrospray ionization (ESI) in positive mode using Waters SQD mass spectrometer (Milford, MA, USA) and evaporative light scattering detector.

LCMS method:
Instrument Information: HPLC/MS-Agilent 1100
Column: Agela Technologies Durashell C18 3.5 m, 100 Å, 4.6×50 mm
Mobile Phase A: Water/0.1% Trifluoroacetic Acid
Mobile Phase B: Acetonitrile/0.1% Trifluoroacetic Acid
Flow Rate: 1 mL/min
Gradient: 70% B to 100% B in 5 minutes, hold 100% B for 10 minutes, 100% B to 70% B in minute, and then stop.
Column Temperature: Ambient
Detector: ELSD

The procedures described below are useful in the synthesis of lipids of Table 1.

The following abbreviations are employed herein:
THF: Tetrahydrofuran
TLC: Thin layer chromatography
MeCN: Acetonitrile
LAH: Lithium Aluminum Hydride
DCM: Dichloromethane
DMAP: 4-Dimethylaminopyridine
LDA: Lithium Diisopropylamide
rt: Room Temperature
DME: 1,2-Dimethoxyethane
n-BuLi: n-Butyllithium
CPME: Cyclopentyl methyl ether
i-Pr₂EtN: N,N-Diisopropylethylamine

### Preparation of 8-(heptadecan-9-yloxy)-N-(2-hydroxyethyl)-8-oxo-N,N-bis(6-oxo-6-(undecyloxy)hexyl)octan-1-aminium (Compound 1)

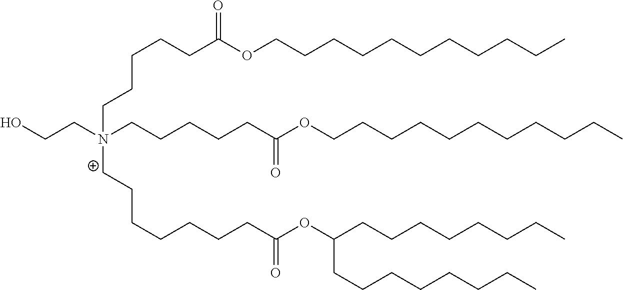

Heptadecan-9-yl 8-((2-hydroxyethyl)(6-oxo-6-(undecyloxy)hexyl)amino)-octanoate (40 g, 51 mmol) and undecyl 6-bromohexanoate (90 g, 258 mmol) were dissolved into acetonitrile (220 mL). The flask was equipped with an air-cooled reflux condenser and stirred at 90° C. The solution was stirred under reflux under N₂ (g). After 5 days, the reaction was cooled down to room temperature and the acetonitrile was dried. The residual oil was dissolved in heptane (600 mL) and the heptane solution was extracted with acetonitrile (3×250 mL). The heptane layer was collected and dried. Portion of the crude oil (4.84 g) was purified by two silica gel column chromatography. First the column was run with 100% isopropyl acetate for multiple column volumes followed by 20% MeOH in dichloromethane. Second column was ran using gradient method [0-100% (mixture of 1% NH₄OH, 20% MeOH in dichloromethane) in dichloromethane] to obtain 998.4 mg of 8-(heptadecan-9-yloxy)-N-(2-hydroxyethyl)-8-oxo-N,N-bis(6-oxo-6-(undecyloxy)hexyl)octan-1-aminium. UPLC/ELSD: RT=3.13 min. HRMS (ESI): m/z calculated for C₆₁H₁₂NO₇⁺ (M+H), 978.91; observed, 979.40. ¹H NMR (300 MHz, CDCl3) δ: ppm 4.85 (m, 1H); 4.49-4.40 (br, m, 1H); 4.19-4.09 (br, 2H); 4.05 (t, 4H, J=6.0 Hz); 3.64-3.57 (br, m, 2H); 3.45-3.31 (br, m, 6H); 2.45-2.23 (m, 6H); 1.83-1.55 (m, 19H); 1.54-1.14 (m, 64H); 0.98-0.82 (m, 15H).

Compound heptadecan-9-yl 8-((2-hydroxyethyl)(6-oxo-6-(undecyloxy)hexyl)amino)octanoate and undecyl 6-bromohexanoate were prepared as described in Sabnis, S.; Kumarasinghe, E. S.; Salerno, T.; Cosmin, M.; Ketoba, T.; Senn, J. J.; Lynn, A.; Bulychev, A.; Mcfadyen, I.; Chan, J.; Almarsson, Ö.; Stanton, M. G. A Novel Amino Lipid Series for mRNA Delivery: Improved Endosomal Escape and Sustained Pharmacology and Safety in Non-human Primates, Molecular Therapy, 2018, 1509-1519; incorporated herein by reference in it's entirety.

### Preparation of 8-(heptadecan-9-yloxy)-N-(2-hydroxyethyl)-N,N-bis(8-(nonyloxy)-8-oxooctyl)-8-oxooctan-1-aminium (Compound 2)

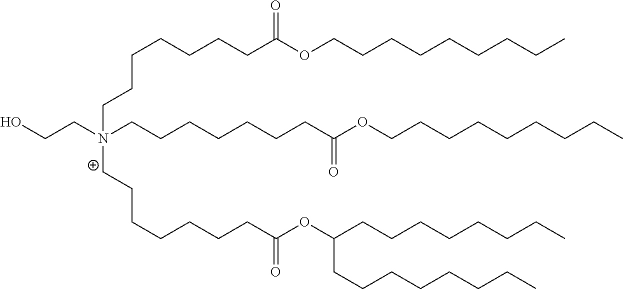

A 12 L three neck round bottom flask equipped with an overhead stirrer, heating mantle, temperature probe, air-cooled reflux condenser, and N₂ inlet was charged with potassium carbonate (225 g, 1.63 mol), potassium iodide (74 g, 0.45 mol) and nonyl 8-((2-hydroxyethyl)amino)octanoate (215 g, 0.40 mol). The solids were suspended in acetonitrile (2.2 L). Nonyl 8-bromooctanoate (167.3 g, 0.47 mol) was added to the stirred suspension, followed by cyclopentyl methyl ether (2200 mL). The resulting mixture was heated at 81.0° C. After 48 h, the reaction was cooled down to room temperature and white inorganic salts were filtered. The filtrate was concentrated to an oil and the crude oil was dissolved in heptane (3 L) and washed with acetonitrile (5×1000 mL). The acetonitrile layer was collected and concentrated. A portion (2 g) of the crude product was purified by two silica gel column chromatography. First silica gel column used gradient method [0-80% (mixture of 1% NH₄OH, 20% MeOH in dichloromethane) in dichloromethane] and the second column was ran with 100% isopropyl acetate for multiple column volumes followed by mixture of 1% NH₄OH, 20% MeOH in dichloromethane. The desired product 8-(heptadecan-9-yloxy)-N-(2-hydroxyethyl)-N,N-bis(8-(nonyloxy)-8-oxooctyl)-8-oxooctan-1-aminium was obtained as light yellow oil (988.4 mg). UPLC/ELSD: RT=3.16 min. IRMS (ESI): m/z calculated for C61H₁₂₀NO₇⁺ (M+H), 978.91; observed, 979.64. ¹H NMR (300 MHz, CDCl3) δ: ppm 4.85 (m, 1H); 4.57-4.36 (br, m, 1H); 4.18-4.09 (br, 2H); 4.05 (t, 4H, J=6.0 Hz); 3.65-3.55 (br, m, 2H); 3.45-3.30 (br, m, 6H); 2.37-2.22 (m, 6H); 1.75-1.55 (m, 19H); 1.54-1.15 (m, 64H); 0.97-0.82 (m, 15H).

Compound nonyl 8-((2-hydroxyethyl)amino)octanoate and nonyl 8-bromooctanoate were prepared as described in Sabnis, S.; Kumarasinghe, E. S.; Salerno, T.; Cosmin, M.; Ketoba, T.; Senn, J. J.; Lynn, A.; Bulychev, A.; Mcfadyen, I.; Chan, J.; Almarsson, Ö.; Stanton, M. G. A Novel Amino Lipid Series for mRNA Delivery: Improved Endosomal Escape and Sustained Pharmacology and Safety in Non-human Primates, Molecular Therapy, 2018, 1509-1519; incorporated herein by reference in it's entirety.

### Preparation of 8-((3-butylheptyl)oxy)-N-(8-(heptadecan-9-yloxy)-8-oxooctyl)-N-(2-hydroxyethyl)-N-(8-(nonyloxy)-8-oxooctyl)-8-oxooctan-1-aminium (Compound 3)

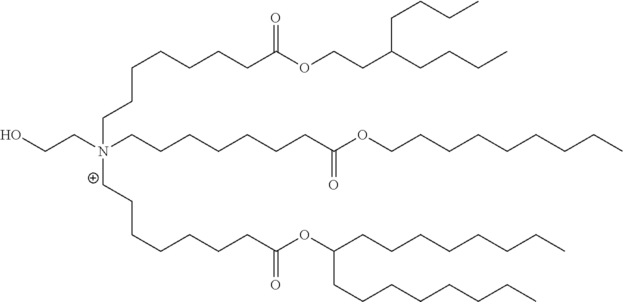

Heptadecan-9-yl 8-((2-hydroxyethyl)(8-(nonyloxy)-8-oxooctyl)amino)octanoate (2 g, 2.82 mmol) and 3-butylheptyl 8-bromooctanoate (5.31 g, 14.08 mmol) were dissolved into acetonitrile (10 mL). The reaction was stirred at 80° C. for 6 days. The reaction change to light yellow orange after a day and light yellow eventually. The reaction was cooled down to room temperature. Heptane (10 mL) was added to the solution and the heptane layer was washed with acetonitrile (6×10 mL). The acetonitrile layer was concentrated and purified by two silica gel column chromatography. First silica gel column used 100% isopropyl acetate for multiple column volumes followed by mixture of 1% NH₄OH, 20% MeOH in dichloromethane. The second silica gel chromatography used gradient method [0-80% (mixture of 1% NH₄OH, 20% MeOH in dichloromethane) in dichloromethane to yield the product (1.06 g), 8-((3-butylheptyl)oxy)-N-(8-(heptadecan-9-yloxy)-8-oxooctyl)-N-(2-hydroxyethyl)-N-(8-(nonyloxy)-8-oxooctyl)-8-oxooctan-1-aminium. UPLC/ELSD: RT=3.07 min. HRMS (ESI): m/z calculated for C₆₃H₁₂₄NO₇⁺ (M+H), 1006.94; observed, 1007.40. ¹H NMR (300 MHz, CDCl3) δ: ppm 4.85 (m, 1H); 4.17-4.00 (m, 7H); 3.59-3.50 (br, m, 2H); 3.45-3.33 (br, m, 6H); 2.35-2.22 (m, 6H); 1.76-1.45 (m, 23H); 1.44-1.17 (m, 64H); 0.96-0.82 (m, 15H).

Compound heptadecan-9-yl 8-((2-hydroxyethyl)(8-(nonyloxy)-8-oxooctyl)amino)octanoate was prepared as described in Sabnis, S.; Kumarasinghe, E. S.; Salerno, T.; Cosmin, M.; Ketoba, T.; Senn, J. J.; Lynn, A.; Bulychev, A.; Mcfadyen, I.; Chan, J.; Almarsson, Ö.; Stanton, M. G. A Novel Amino Lipid Series for mRNA Delivery: Improved Endosomal Escape and Sustained Pharmacology and Safety in Non-human Primates, Molecular Therapy, 2018, 1509-1519; and compound 3-butylheptyl 8-bromooctanoate was prepared as described in Benenato, K. E.; Cornebise, M.; Hennessy, E.; Kumarasinghe, E. S. Branched Tail Lipid Compounds and Compositions for Intracellular Delivery of Therapeutic Agents, U.S. Ser. No. 11/066,355B2; incorporated herein by reference in it's entirety.

### Preparation of 8-((3-butylheptyl)oxy)-N-(8-((3-butylheptyl)oxy)-8-oxooctyl)-N-(8-(heptadecan-9-yloxy)-8-oxooctyl)-N-(2-hydroxyethyl)-8-oxooctan-1-aminium (Compound 4)

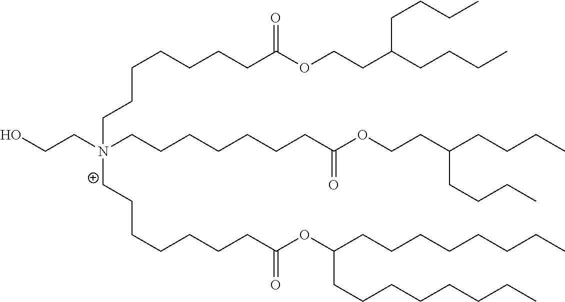

To a round bottom flask, heptadecan-9-yl 8-((2-hydroxyethyl)(8-(nonyloxy)-8-oxooctyl)amino)octanoate (2 g, 2.709 mmol) and 3-butylheptyl 8-bromooctanoate (5.112 g, 13.55 mmol) was dissolved into acetonitrile (10 mL). The reaction was stirred at 80° C. for 6 days, where the reaction color changed from clear to light yellow. After the reaction was cooled down to room temperature, 10 mL of heptane was added to the solution. The heptane layer was washed with acetonitrile (6×10 mL). The acetonitrile fractions were collected and concentrated to be purified by silica gel chromatography. First silica gel column was ran with 100% isopropyl acetate for multiple column volumes followed by mixture of 1% NH₄OH, 20% MeOH in dichloromethane. After the first column, a second silica gel chromatography used gradient method [0-80% (mixture of 1% NH₄OH, 20% MeOH in dichloromethane) in dichloromethane to collect the targeted product, 8-((3-butylheptyl)oxy)-N-(8-((3-butylheptyl)oxy)-8-oxooctyl)-N-(8-(heptadecan-9-yloxy)-8-oxooctyl)-N-(2-hydroxyethyl)-8-oxooctan-1-aminium (1.274 g). UPLC/ELSD: RT=3.08 min. IRMS (ESI): m/z calculated for C₆₅H₁₂₈NO₇⁺ (M+H), 1034.97; observed, 1035.03. ¹H NMR (300 MHz, CDCl3) δ: ppm 4.85 (m, 1H); 4.20-4.01 (m, 6H); 3.56-3.50 (br, m, 2H); 3.45-3.32 (br, m, 6H); 2.35-2.21 (m, 6H); 1.75-1.46 (m, 25H); 1.45-1.17 (m, 64H); 0.98-0.81 (m, 18H).

Compound heptadecan-9-yl 8-((2-hydroxyethyl)(8-(nonyloxy)-8-oxooctyl)amino)-octanoate and 3-butylheptyl 8-bromooctanoate were prepared as described in Benenato, K. E.; Cornebise, M.; Hennessy, E.; Kumarasinghe, E. S. Branched Tail Lipid Compounds and Compositions for Intracellular Delivery of Therapeutic Agents, U.S. Ser. No. 11/066,355B2; incorporated herein by reference in it's entirety.

### Example 2: LNP Formulations

Lipid nanoparticles (e.g., empty LNPs or loaded LNPs) including a therapeutic and/or prophylactic can be optimized according to the selection of a cationic lipid according to Formula (I), the selection of additional lipids, the amount of each lipid in the lipid component, and the wt:wt ratio of the lipid component to the therapeutic and/or prophylactic.

Lipid nanoparticles (e.g., empty LNPs or loaded LNPs), including DSPC and DOPE as a phospholipid, cholesterol as a structural lipid, PEG-1 as a PEG lipid, an ionizable lipid according to Formula (IL-A), (IL-B), (IL-C), (IL-D), (IL-I), (IL-IA), (IL-IB), (IL-IC), (IL-IIA), (IL-IIAX), (IL-IIB), (IL-IIC), (IL-III), or (IL-IIIA), and a cationic lipid according to Formula (I) were prepared.

Exemplary lipid nanoparticle compositions were prepared by dissolving lipids (e.g., an ionizable lipid according to Formula (TL-A), (IL-B), (TL-C), (TL-D), (IL-I), (IL-IA), (IL-IB), (IL-IC), (IL-IIA), (IL-IIAX), (IL-IIB), (IL-IIC), (IL-III), or (IL-IIIA), a cationic lipid according to Formula (I), DSPC, cholesterol and PEG 1) in ethanol at a concentration of 12.5 mM and molar ratios as summarized in Table 2. The lipid to mRNA ratio was maintained at a N/P ratio of 4.9. The mRNA was then diluted with 25 mM sodium acetate (pH 5.0) and combined with the lipid mixture at a volume ratio of 3:1 (aqueous:ethanol). Resulting formulations were dialyzed against 20 mM tris/8% sucrose mM sodium chloride (pH 7.4) at a volume of 300 times that of the primary product, using Slide-A-Lyzer dialysis cassettes (Thermo Scientific, Rockford, IL, USA) with a molecular cutoff of 10 KDa for at least 18 h. The first dialysis was carried out at room temperature in a digital orbital shaker at 85 rpm for 3 h and then subsequently dialyzed overnight at 4° C. Formulations were concentrated using centrifugal filters, passed through a 0.22-μm filter and stored at 4° C. until use. Lipid nanoparticle solutions were typically adjusted to specific mRNA concentrations between 0.1 mg/mL and 1 mg/mL.

Table 2 summarizes the components and compositions of exemplary LNPs. Table 3 summarizes the characteristics of the LNP formulations, all of which were determined on the day of preparation and at room temperature. As shown in Table 3 the majority of LNPs comprising the cationic lipid of Formula (I) presented a small size (between 60 and 80 nm).

TABLE 2
Components and composition of exemplary LNPs.

| | Cationic | Ionizable | | Structural | | |
|---|---|---|---|---|---|---|
| | Lipid | Lipid | Phospholipid | lipid | PEG lipid | |
| LNP | (% mol) | (% mol) | (% mol) | (% mol) | (% mol) | N:P |
| LNP-1 | — | II-6 (48) | DSPC (11.0) | Cholesterol | PEG-1 (2.0) | 4.9 |
| | | | | (39) | | |
| LNP-2 | 1 (30) | VI-4 (25) | DOPE (16.7) | Cholesterol | PEG-DMG (1.5) | 4.9 |
| | | | | (26.8) | | |
| LNP-3 | 1 (30) | II-6 (33) | DSPC (7.7) | Cholesterol | PEG-1 (2.0) | 4.9 |
| | | | | (27.3) | | |
| LNP-4 | 2 (30) | II-6 (33) | DSPC (7.7) | Cholesterol | PEG-1 (2.0) | 4.9 |
| | | | | (27.3) | | |
| LNP-5 | 3 (30) | II-6 (33) | DSPC (7.7) | Cholesterol | PEG-1 (2.0) | 4.9 |
| | | | | (27.3) | | |
| LNP-6 | 4 (30) | II-6 (33) | DSPC (7.7) | Cholesterol | PEG-1 (2.0) | 4.9 |
| | | | | (27.3) | | |

TABLE 3
Characteristics of nanoparticles comprising lipids of the disclosure.

| | Cationic | | Diameter/ | | |
|---|---|---|---|---|---|
| LNP | Lipid (% mol) | mRNA | Size (nm) | PDI | N:P |
| LNP-1 | — | mOX40L | 56.7 | 0.154 | 4.9 |
| LNP-2 | 1 (30) | mOX40L | 64.1 | 0.182 | 4.9 |
| LNP-3 | 1 (30) | mOX40L | 68.9 | 0.299 | 4.9 |
| LNP-4 | 2 (30) | mOX40L | 70.6 | 0.331 | 4.9 |
| LNP-5 | 3 (30) | mOX40L | 79.1 | 0.136 | 4.9 |
| LNP-6 | 4 (30) | mOX40L | 146.5 | Multimodal | 4.9 |

### Example 3: Isolation of Murine Endothelial Lung Cells for Flow Cytometry

Reagents: Digestion Media: 3 mg/mL collagenase 1, 0.1 mg/mL DNase I, Dulbecco's Modified Eagle Medium (DMEM); Wash solution: Dulbecco's phosphate-buffered saline (DPBS)+0.5% Bovine Serum Albumin (BSA); FACS Buffer: Flow Cytometry Staining Buffer (eBioscience™, ThermoFisher Scientific).

### Minimum Marker Panel:

CD31: General Endothelial Marker;
CD45: Leukocyte Common Marker;
mOX40L: Transmembrane Reporter;
tdTomato: Fluorescence Protein Reporter from Cre-mediated recombination in Ail4 mice.

Six-week-old female mice (Ail4) were aged in-house to approximately 7-8 weeks of age. With an n=5 per group, Cre mRNA (i.e., mRNA leading to expression of tdTomato) formulated LNPs were intravenously dosed via the lateral tail vein 8 days before harvest. A week after the first dose, mice were intravenously (lateral tail vain) dosed with an loaded LNP of the disclosure comprising an mRNA expressing OX40L. Twenty-four hours after the second intravenous dose, mice were euthanized under CO₂ asphyxiation and the right atrium snipped to allow blood to perfuse out. The lungs were perfused with 5 mL PBS through the right ventricle of the heart and subsequently removed. The left lobe of the lung was stored in 3 mL PBS and placed on ice. The left lung lobes were cut into <1 mm pieces and each placed in 8 mL of digestion media. The tissue suspension was incubated in digestion media for 45 minutes at 37° C. with inversion every 15 minutes. The tissue suspension was passed through a 3 mL syringe with 20 G cannula attached until the mixture was triturated into a single cell suspension. The cell mixture was filtered through a 70 μm strainer into ice cold wash solution. Additional wash solution was subsequently added on top of strainer. The cell mixture was centrifuged at 300×g, 4° C. for 5 minutes. The supernatant was removed, and the cell pellet was resuspended in wash buffer. The cell mixture was centrifuged at 300×g, 4° C. for 5 minutes. The supernatant was removed, and the cell pellet was resuspended in Ammonium-Chloride-Potassium (ACK) lysis buffer for 1 minute and wash buffer was added (2× volume). The cell mixture was centrifuged at 300×g, 4° C. for 5 minutes and the supernatant subsequently removed. The cell pellet was resuspended in Flow Cytometry Staining (FACS) buffer and filtered through a 70 μm mesh. Lung cells were stained with viability dye and antibodies against the marker panel according to manufacturers' recommendations. Cells were resuspended in an anti-mouse CD16/32 antibody (TruStain FcX™, BioLegend) 10 minutes before staining with antibody cocktail. Compensation controls were run on flow cytometer. Lung samples were run on an acoustic focusing flow cytometer (Attune™ NxT, ThermoFisher Scientific). Collected data was analyzed using flow cytometry analysis software (FlowJo™).

### Example 4: Endothelial Delivery by LNPs of the Disclosure

To assess the delivery of a therapeutic agent comprised in loaded LNPs of the disclosure to endothelial cells, the expression of Cre and mOX40L mRNA encapsulated in loaded LNPs of the disclosure was measured in endothelial cells following administration of the loaded LNPs to Ail4 mice as described in Example 3. The results are summarized in Tables 4 and 5 below, and show the fraction of endothelial cells that exhibit expression following administration of an LNP of the disclosure in a sample endothelial cell population that is positive for a given reporter as detected by flow cytometry. The expression was evaluated in LNPs comprising 30 mol % of a cationic lipid of Formula (I) and in loaded LNPs not comprising the cationic of Formula (I) (i.e., LNPs not comprising a cationic lipid, or LNPs comprising dioleoyl-3-trimethylammonium propane (DOTAP)). The loaded LNPs also comprised an ionizable lipid and a PEG lipid as indicated in the tables.

TABLE 4
Expression of mOX40L in endothelial cells following
administration of loaded LNPs of the disclosure

| Ionizable | Cationic | % Endothelial cells | | | | | |
|---|---|---|---|---|---|---|---|
| lipid/PEG lipid | lipid | Sample 1 | Sample 2 | Sample 3 | Sample 4 | Sample 5 | Average |
| VI-4/PEG-DMG | None | | | | | | 0 |
| | 1 | 77.3 | 74.0 | 70.3 | 79.8 | 77.9 | 75.9 |
| | DOTAP | | | | | | 0 |
| I-18/ PEG-1 | None | | | | | | 0 |
| | 1 | | | | | | 0 |
| II-6/ PEG-1 | None | 0.4 | 0.4 | 0.9 | 0.4 | 0.4 | 0.5 |
| | 1 | 79.6 | 61.8 | 76.9 | 76.6 | 78.0 | 74.6 |
| | 2 | 62.2 | 83.2 | 71.2 | 89.1 | 79.2 | 77.0 |
| | 3 | 63.5 | 72.4 | 87.4 | 83.8 | 88.0 | 79.0 |
| | 4 | 65.2 | 78.5 | 80.7 | 58.1 | 80.5 | 72.6 |

TABLE 5
Expression of tdTomato in endothelial cells following
administration of loaded LNPs of the disclosure

| Ionizable | Cationic | % Endothelial cells | | | | | |
|---|---|---|---|---|---|---|---|
| lipid/PEG lipid | lipid | Sample 1 | Sample 2 | Sample 3 | Sample 4 | Sample 5 | Average |
| VI-4/PEG-DMG | None | | | | | | 0 |
| | 1 | 82.1 | 74.9 | 80.8 | 77.7 | 79.4 | 79.0 |
| | DOTAP | | | | | | 0 |
| I-18/ PEG-1 | None | | | | | | 0 |
| | 1 | | | | | | 0 |
| II-6/ PEG-1 | None | 7.0 | 4.1 | 5.0 | 2.7 | 3.2 | 4.4 |
| | 1 | 82.4 | 76.2 | 71.9 | 73.6 | 74.5 | 75.7 |
| | 2 | 83.2 | 73.9 | 76.5 | 79.6 | 80.4 | 78.7 |
| | 3 | 76.1 | 75.3 | 76.5 | 76.2 | 76.2 | 76.1 |
| | 4 | 69.4 | 72.2 | 69.2 | 70.2 | 70.9 | 70.4 |

### ENUMERATED EMBODIMENTS

Embodiment 1. A cationic lipid of Formula (I):

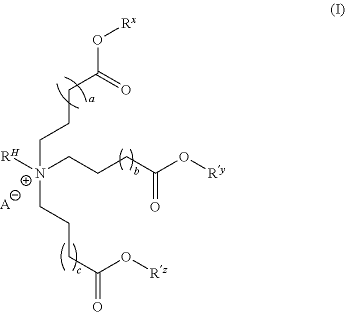

or an isomer thereof,
   wherein R′^{x} is

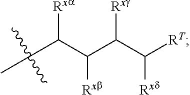

R′^{y} is

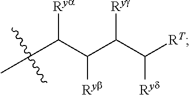

and R′^{z} is

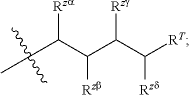

wherein

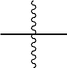

denotes a point of attachment;
R^{xα}, R^{xγ}, R^{xγ}, and R^{xδ} are each independently selected from the group consisting of H, C₁₋₁₂ alkyl, and C₂₋₁₂ alkenyl;
R^{yα}, R^{yβ}, R^{yγ}, and R^{yδ} are each independently selected from the group consisting of H, C₁₋₁₂ alkyl, and C₂₋₁₂ alkenyl;
R^{zα}, R^{zβ}, R^{zγ}, and R^{zδ} are each independently selected from the group consisting of H, C₁₋₁₂ alkyl, and C₂₋₁₂ alkenyl;
R^{H} is —(CH₂)_{q}OH, wherein q is selected from 1, 2, 3, 4, and 5;
each R^{T} is independently selected from C₁₋₁₂ alkyl and C₂₋₁₂ alkenyl;
a is selected from 1, 2, 3, 4, 5, 6, 7, 8, and 9;
b is selected from 1, 2, 3, 4, 5, 6, 7, 8, and 9;
c is selected from 1, 2, 3, 4, 5, 6, 7, 8, and 9; and
A⁻ is any pharmaceutically acceptable anion.
Embodiment 2. A cationic lipid of Formula (I-cat):

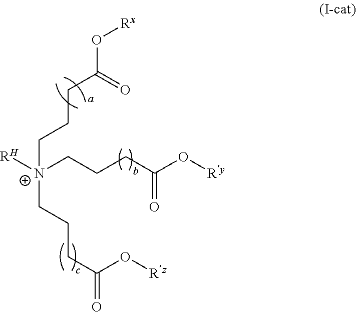

or an isomer thereof,
   wherein R″^{x} is

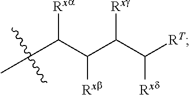

R′^{y} is

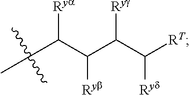

and R′z is

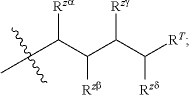

wherein

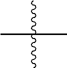

denotes a point of attachment;
R^{xα}, R^{xγ}, R^{xγ}, and R^{xδ} are each independently selected from the group consisting of H, C₁₋₁₂ alkyl, and C₂₋₁₂ alkenyl;
R^{yα}, R^{yβ}, R^{yγ}, and R^{yδ} are each independently selected from the group consisting of H, C₁₋₁₂ alkyl, and C₂₋₁₂ alkenyl;
R^{zα}, R^{zβ}, R^{zγ}, and R^{zδ} are each independently selected from the group consisting of H, C₁₋₁₂ alkyl, and C₂₋₁₂ alkenyl;
R^{H} is —(CH₂)_{q}OH, wherein q is selected from 1, 2, 3, 4, and 5;
each R^{T} is independently selected from C₁₋₁₂ alkyl and C₂₋₁₂ alkenyl;
a is selected from 1, 2, 3, 4, 5, 6, 7, 8, and 9;
b is selected from 1, 2, 3, 4, 5, 6, 7, 8, and 9;
c is selected from 1, 2, 3, 4, 5, 6, 7, 8, and 9.
Embodiment 3. The cationic lipid of embodiment 1 or 2, wherein
   R′^{x} is:

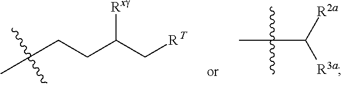

R′^{y} is:

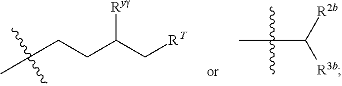

and R′^{z} is:

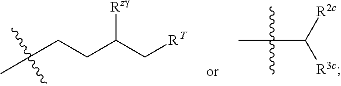

wherein

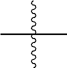

denotes a point of attachment;
R^{xγ} is selected from the group consisting of H, C₁₋₁₂ alkyl, and C₂₋₁₂ alkenyl;
R^{yγ} is selected from the group consisting of H, C₁₋₁₂ alkyl, and C₂₋₁₂ alkenyl;
R^{zγ} is selected from the group consisting of H, C₁₋₁₂ alkyl, and C₂₋₁₂ alkenyl; and
R^{2a}, R^{2b}, R^{2c}, R^{3a}, R^{3b}, and R^{3c} are each independently selected from the group consisting of C₁₋₁₄ alkyl and C₂₋₁₄ alkenyl.
Embodiment 4. The cationic lipid of any one of the preceding embodiments, wherein
   R′^{a} is

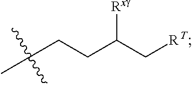

R′^{b} is

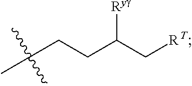

and R′^{c} is

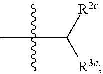

wherein

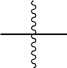

denotes a point of attachment;
R^{xγ} is selected from the group consisting of H, C₁₋₁₂ alkyl, and C₂₋₁₂ alkenyl;
R^{yγ} Tis selected from the group consisting of H, C₁₋₁₂ alkyl, and C₂₋₁₂ alkenyl; and
R^{2c} and R^{3c} are each independently selected from the group consisting of C₁₋₁₄ alkyl and C₂₋₁₄ alkenyl.
Embodiment 5. The compound of any one of the preceding embodiments, wherein R^{xγ} and R^{yγ} are each H.
Embodiment 6. The compound of any one of the preceding embodiments, wherein R^{xγ} and R^{yγ} are each C₁₋₁₂ alkyl or C₂₋₁₂ alkenyl.
Embodiment 7. The compound of any one of the preceding embodiments, wherein R^{X7} is C₁₋₁₂ alkyl or C₂₋₁₂ alkenyl and R^{yγ} is H.
Embodiment 8. The compound of any one of the preceding embodiments, wherein q is 2.
Embodiment 9. The compound of any one of the preceding embodiments, wherein the compound is selected from:

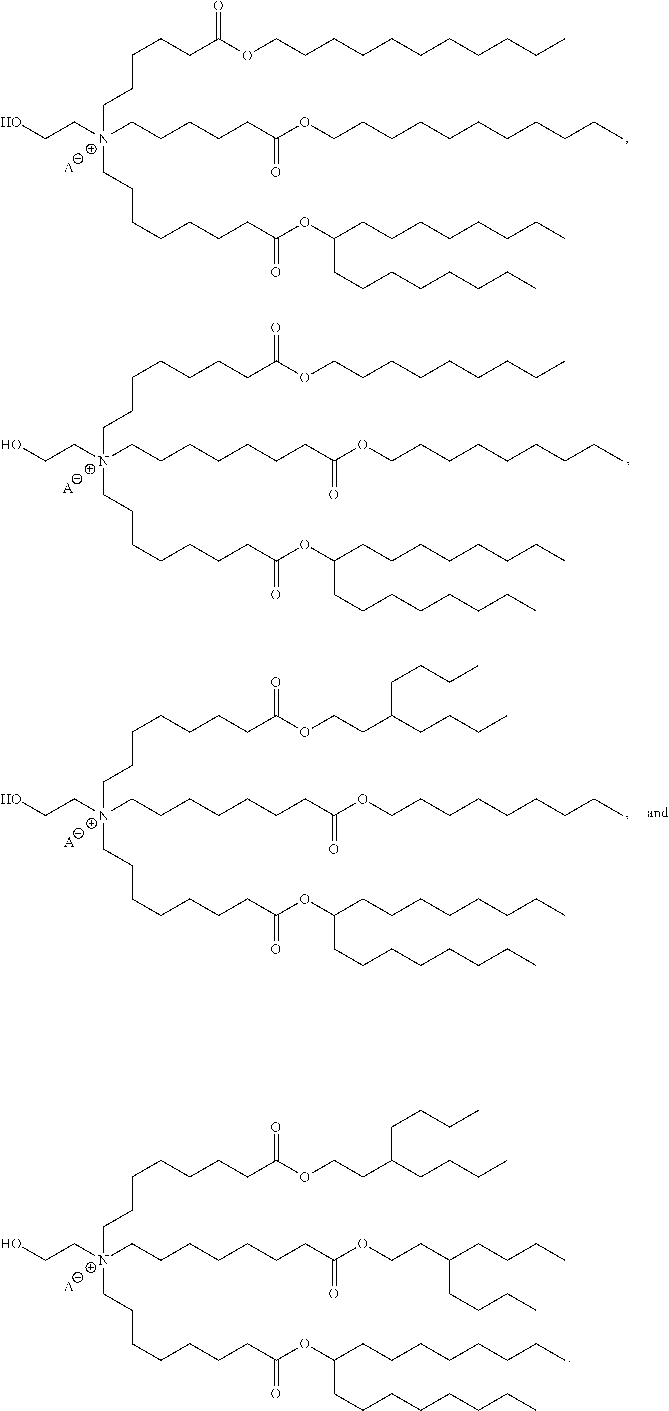

Embodiment 10. A compound of selected from:

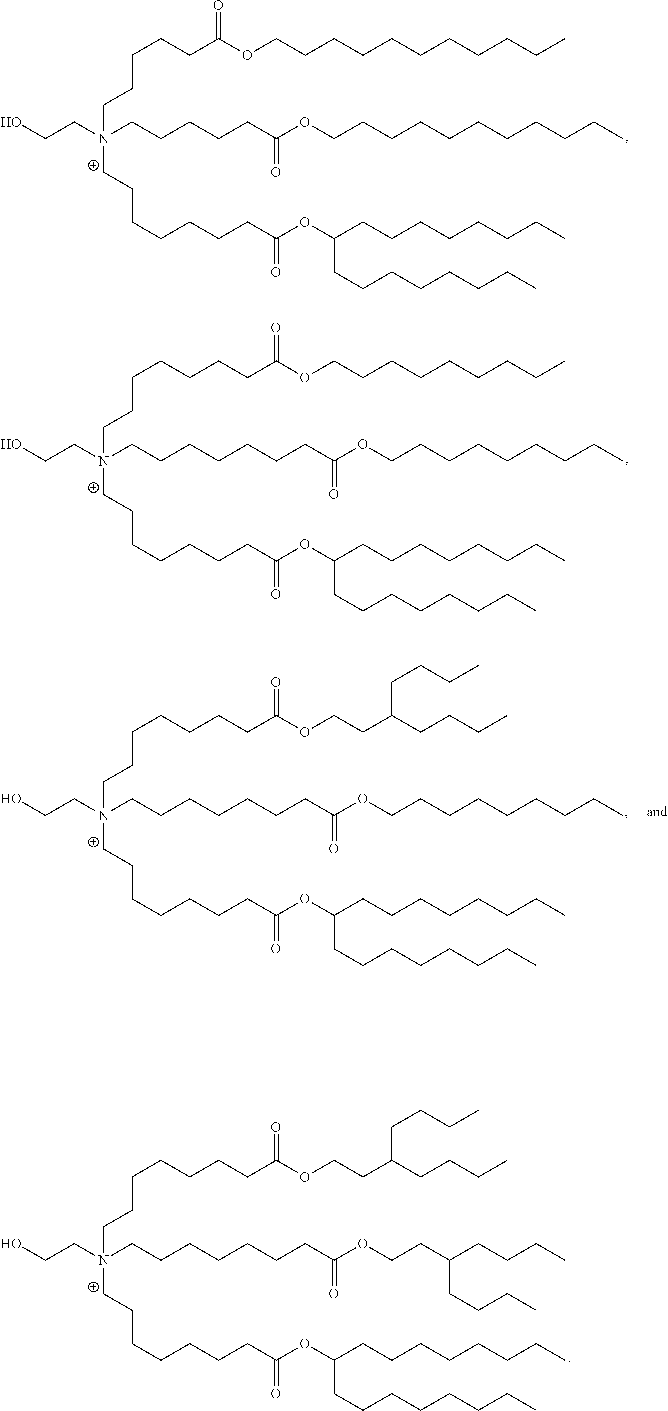

Embodiment 11. The compound of any one of the preceding embodiments, wherein A⁻ is selected from chloride, bromide, iodide, hydroxide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate.
Embodiment 12. The compound of any one of the preceding embodiments, wherein A⁻ is selected from bromide, chloride, and hydroxide.
Embodiment 13. The compound of any one of the preceding embodiments, wherein A⁻ is bromide.
Embodiment 14. The compound of any one of the preceding embodiments, wherein A⁻ is chloride.
Embodiment 15. The compound of any one of the preceding embodiments, wherein A⁻ is bromide or hydroxide.
Embodiment 16. The compound of any one of the preceding embodiments, wherein A⁻ is chloride or hydroxide.
Embodiment 17. The compound of any one of the preceding embodiments, wherein A⁻ is hydroxide.
Embodiment 18. An empty lipid nanoparticle (empty LNP) comprising a cationic lipid of any one of the preceding embodiments.
Embodiment 19. The empty LNP of any one of the preceding embodiments, further comprising an ionizable lipid.
Embodiment 20. The empty LNP of any one of the preceding embodiments, further comprising a phospholipid.
Embodiment 21. The empty LNP of any one of the preceding embodiments, further comprising a structural lipid.
Embodiment 22. The empty LNP of any one of the preceding embodiments, further comprising a PEG lipid.
Embodiment 23. An empty LNP comprising a lipid component which comprises from about 20 mol % to about 40 mol % of the cationic lipid of any one of the preceding embodiments; from about 15 mol % to about 40 mol % ionizable lipid, from about 0 mol % to about 30 mol % phospholipid, from about 15 mol % to about 50 mol % structural lipid, and from about 0 mol % to about 1 mol % PEG lipid.
Embodiment 24. A loaded lipid nanoparticle (loaded LNP) comprising the empty LNP of any one of the preceding embodiments and a therapeutic and/or prophylactic agent.
Embodiment 25. A loaded LNP comprising:
   (a) a lipid nanoparticle core,
   (b) a therapeutic and/or prophylactic agent encapsulated within the core for delivery into a cell, and
   (c) a cationic agent disposed primarily on the outer surface of the core, wherein the loaded LNP has a greater than neutral zeta potential at physiologic pH.
Embodiment 26. A loaded LNP comprising:
   (a) a loaded LNP core comprising:
      (i) an ionizable lipid,
      (ii) a phospholipid,
      (iii) a structural lipid, and
      (iv) a PEG-lipid, and
   (b) a therapeutic and/or prophylactic agent encapsulated within the core for delivery into a cell, and
   (c) a cationic agent.
Embodiment 27. A loaded LNP comprising:
(a) a lipid nanoparticle core comprising:
   (i) an ionizable lipid,
   (ii) a phospholipid,
   (iii) a structural lipid, and
   (iv) a PEG-lipid, and
(b) a polynucleotide or polypeptide payloadtherapeutic and/or prophylactic agent encapsulated within the core for delivery into a cell, and
(c) a cationic agent disposed primarily on the outer surface of the core.
Embodiment 28. A loaded LNP comprising:
   (a) a lipid nanoparticle core,
   (b) a therapeutic and/or prophylactic agent encapsulated within the core for delivery into a cell, and
   (c) a cationic agent,
wherein the loaded LNP exhibits a cellular accumulation of at least about 20% of cells and exhibits about 5% or greater expression in cells in a population of cells to which the loaded LNP is administered. In some embodiments, the loaded LNP exhibits a cellular accumulation in from about 1% to about 75%, from about 5% to about 50%, from about 10% to about 40%, or from about 15% to about 25% of cells in a population of cells to which the loaded LNP is administered.
Embodiment 29. The loaded LNP of any one of the preceeding embodiments, wherein the loaded LNP exhibits expression in from about 0.5% to about 50%, from about 1% to about 40%, from about 3% to about 20%, or from about 5% to about 15% of cells in which the loaded LNP is accumulated.
Embodiment 30. In some embodiments aspect, provided herein is a loaded LNP
Embodiment 31. comprising:
   (a) a lipid nanoparticle core,
   (b) a therapeutic and/or prophylactic agent encapsulated within the core for delivery into a cell, and
   (c) a cationic agent disposed primarily on the outer surface of the core,
wherein the loaded LNP exhibits a cellular accumulation in at least about 20% of cells in a population of cells to which the loaded LNP is administered and exhibits about 5% or greater expression in cells in which the loaded LNP is accumulated.
Embodiment 32. The loaded LNP of any one of the preceeding embodiments, wherein the loaded LNP exhibits a cellular accumulation in from about 1% to about 75%, from about 5% to about 50%, from about 10% to about 40%, or from about 15% to about 25% of cells in a population of cells to which the loaded LNP is administered.
Embodiment 33. The loaded LNP of any one of the preceeding embodiments, wherein the loaded LNP exhibits from about 0.5% to about 50%, from about 1% to about 40%, from about 3% to about 20%, or from about 5% to about 15% expression in cells in which the loaded LNP is accumulated.
Embodiment 34. A loaded LNP comprising:
   (a) a lipid nanoparticle core,
   (b) a therapeutic and/or prophylactic agent encapsulated within the core for delivery into a cell, and
   (c) a cationic agent disposed primarily on the outer surface of the core,
wherein the therapeutic and/or prophylactic agent expresses a protein and wherein loaded LNP exhibits protein expression of about 0.5% to 50% in cells in a population of cells to which the loaded LNP is administered.
Embodiment 35. The loaded LNP of any one of the preceeding embodiments, wherein the loaded LNP exhibits protein expression of from about 0.1% to about 60%, from about 0.5% to about 40%, from about 1% to about 30%, or from about 1% to about 20% in cells in which the loaded LNP is accumulated.
Embodiment 36. A loaded LNP comprising:
   (a) a lipid nanoparticle core,
   (b) a therapeutic and/or prophylactic agent encapsulated within the core for delivery into a cell, and
   (c) a cationic agent,
wherein the loaded LNP exhibits protein expression of from about 0.5% to 50% in cells in which the loaded LNP is accumulated.
Embodiment 37. The loaded LNP of any one of the preceeding embodiments, wherein the loaded LNP exhibits protein expression of from about 0.1% to about 60%, from about 0.5% to about 40%, from about 1% to about 30%, or from about 1% to about 20% in cells in which the loaded LNP is accumulated.
Embodiment 38. A loaded LNP comprising:
   (a) a lipid nanoparticle core,
   (b) a therapeutic and/or prophylactic agent encapsulated within the core for delivery into a cell, and
   (c) a cationic agent,
wherein the loaded LNP exhibits a cellular accumulation of at least about 20% in endothelial cells and exhibits about 5% or greater expression in endothelial cells in which the loaded LNP is accumulated.
Embodiment 39. The loaded LNP of any one of the preceeding embodiments, wherein the loaded LNP exhibits a cellular accumulation of from about 1% to about 75%, from about 5% to about 50%, from about 10% to about 40%, or from about 15% to about 25% of endothelial cells in a population of endothelial cells cells to which the loaded LNP is administered.
Embodiment 40. The loaded LNP of any one of the preceeding embodiments, wherein the loaded LNP exhibits from about 0.5% to about 50%, from about 1% to about 40%, from about 3% to about 20%, or from about 5% to about 15% expression in endothelial cells in which the loaded LNP is accumulated.
Embodiment 41. A loaded LNP comprising:
   (a) a lipid nanoparticle core,
   (b) a therapeutic and/or prophylactic agent encapsulated within the core for delivery into a cell, and
   (c) a cationic agent,
wherein the loaded LNP exhibits protein expression of from about 0.5% to 50% of endothelial cells in which the loaded LNP is accumulated.
Embodiment 42. The loaded LNP of any one of the preceeding embodiments, wherein the loaded LNP exhibits protein expression of from about 0.1% to about 60%, from about 0.5% to about 40%, from about 1% to about 30%, or from about 1% to about 20% of endothelial cells in which the loaded LNP is accumulated.
Embodiment 43. A loaded LNP comprising:
   (a) a lipid nanoparticle core,
   (b) a therapeutic and/or prophylactic agent encapsulated within the core for delivery into a cell, and
   (c) a cationic agent,
wherein the loaded LNP exhibits protein expression in from about 0.5% to about 50% of lung cells in which the loaded LNP is accumulated.
Embodiment 44. The loaded LNP of any one of the preceeding embodiments, wherein the loaded LNP exhibits protein expression of from about 0.1% to about 60%, from about 0.5% to about 40%, from about 1% to about 30%, or from about 1% to about 20% of pulmonary endothelial cells in which the loaded LNP is accumulated.
Embodiment 45. A loaded LNP comprising:
   (a) a lipid loaded LNP core,
   (b) a therapeutic and/or prophylactic agent encapsulated within the core for delivery into a cell, and
   (c) a cationic agent,
wherein the loaded LNP exhibits a cellular accumulation of at least about 20% in respiratory endothelial cells and exhibits about 5% or greater expression in respiratory endothelial cells in which the loaded LNP is accumulated.
Embodiment 46. The loaded LNP of any one of the preceeding embodiments, wherein the loaded LNP exhibits a cellular accumulation of from about 1% to about 75%, from about 5% to about 50%, from about 10% to about 40%, or from about 15% to about 25% of respiratory endothelial cells in a population of respiratory endothelial cells to which the loaded LNP is administered.
Embodiment 47. The loaded LNP of any one of the preceeding embodiments, wherein the loaded LNP exhibits from about 0.5% to about 50%, from about 1% to about 40%, from about 3% to about 20%, or from about 5% to about 15% expression in respiratory endothelial cells in which the loaded LNP is accumulated.
Embodiment 48. A loaded LNP comprising:
   (a) a lipid loaded LNP core,
   (b) a therapeutic and/or prophylactic agent encapsulated within the core for delivery into a cell, and
   (c) a cationic agent,
wherein the loaded LNP exhibits protein expression in about 0.5% to about 50% respiratory endothelial cells in which the loaded LNP is accumulated.
Embodiment 49. The loaded LNP of any one of the preceeding embodiments, wherein the loaded LNP exhibits protein expression of from about 0.1% to about 60%, from about 0.5% to about 40%, from about 1% to about 30%, or from about 1% to about 20% of respiratory endothelial cells in which the loaded LNP is accumulated.
Embodiment 50. A loaded LNP comprising:
   (a) a lipid loaded LNP core,
   (b) a therapeutic and/or prophylactic agent encapsulated within the core for delivery into a cell, and
   (c) a cationic agent,
wherein the loaded LNP exhibits protein expression in about 0.5% to about 50% of HeLa cells in which the loaded LNP is accumulated.
Embodiment 51. The loaded LNP of any one of the preceeding embodiments, wherein the loaded LNP exhibits protein expression of from about 0.1% to about 60%, from about 0.5% to about 40%, from about 1% to about 30%, or from about 1% to about 20% of HeLa cells in which the loaded LNP is accumulated.
Embodiment 52. A loaded LNP comprising:
   (a) a lipid loaded LNP core,
   (b) a therapeutic and/or prophylactic agent encapsulated within the core for delivery into a cell, and
   (c) a cationic agent,
wherein the loaded LNP exhibits a cellular accumulation of at least about 20% in bronchial endothelial cells in a population of bronchial endothelial cells to which the loaded LNP is administered and exhibits about 5% or greater expression in bronchial endothelial cells in which the loaded LNP is accumulated.
Embodiment 53. The loaded LNP of any one of the preceeding embodiments, wherein the cationic agent is a cationic lipid.
Embodiment 54. The loaded LNP of any one of the preceeding embodiments, wherein the cationic agent is a cationic lipid of Formula (I).
Embodiment 55. The loaded LNP of any one of the preceeding embodiments, wherein the cationic agent is a cationic lipid of any one of Embodiments 1-17.
Embodiment 56. The loaded LNP of any one of the preceeding embodiments, wherein the loaded LNP exhibits a cellular accumulation of from about 1% to about 75%, from about 5% to about 50%, from about 10% to about 40%, or from about 15% to about 25% of respiratory endothelial cells in a population of respiratory endothelial cells to which the loaded LNP is administered.
Embodiment 57. The loaded LNP of any one of the preceeding embodiments, wherein the loaded LNP exhibits from about 0.5% to about 50%, from about 1% to about 40%, from about 3% to about 20%, or from about 5% to about 15% expression in lung endothelial cells in which the loaded LNP is accumulated.
Embodiment 58. The loaded LNP of any one of the preceding embodiments, wherein a weight ratio of the cationic agent to the therapeutic and/or prophylactic agent is from about 0.1:1 to about 15:1.
Embodiment 59. The loaded LNP of any one of the preceding embodiments, wherein a weight ratio of the cationic agent to the therapeutic and/or prophylactic agent is from about 0.2:1 to about 10:1.
Embodiment 60. The loaded LNP of any one of the preceding embodiments, wherein a weight ratio of the cationic agent to the therapeutic and/or prophylactic agent is from about 1:1 to about 10:1.
Embodiment 61. The loaded LNP of any one of the preceding embodiments, wherein a weight ratio of the cationic agent to the therapeutic and/or prophylactic agent is from about 1:1 to about 8:1.
Embodiment 62. The loaded LNP of any one of the preceding embodiments, wherein a weight ratio of the cationic agent to the therapeutic and/or prophylactic agent is from about 1:1 to about 7:1.
Embodiment 63. The loaded LNP of any one of the preceding embodiments, wherein a weight ratio of the cationic agent to the therapeutic and/or prophylactic agent is from about 1:1 to about 6:1.
Embodiment 64. The loaded LNP of any one of the preceding embodiments, wherein a weight ratio of the cationic agent to the therapeutic and/or prophylactic agent is from about 1:1 to about 5:1.
Embodiment 65. The loaded LNP of any one of the preceding embodiments, wherein a weight ratio of the cationic agent to the therapeutic and/or prophylactic agent is from about 1:1 to about 4:1.
Embodiment 66. The loaded LNP of any one of the preceding embodiments, wherein a weight ratio of the cationic agent to the therapeutic and/or prophylactic agent is from about 1.25:1 to about 3.75:1.
Embodiment 67. The loaded LNP of any one of the preceding embodiments, wherein a weight ratio of the cationic agent to the therapeutic and/or prophylactic agent is from about 1.25:1.
Embodiment 68. The loaded LNP of any one of the preceding embodiments, wherein a weight ratio of the cationic agent to the therapeutic and/or prophylactic agent is from about 2.5:1.
Embodiment 69. The loaded LNP of any one of the preceding embodiments, wherein a weight ratio of the cationic agent to the therapeutic and/or prophylactic agent is from about 3.75:1.
Embodiment 70. The loaded LNP of any one of the preceding embodiments, wherein a molar ratio of the cationic agent to the therapeutic and/or prophylactic agent is from about 0.1:1 to about 20:1.
Embodiment 71. The loaded LNP of any one of the preceding embodiments, wherein a molar ratio of the cationic agent to the therapeutic and/or prophylactic agent is from about 1.5:1 to about 10:1.
Embodiment 72. The loaded LNP of any one of the preceding embodiments, wherein a molar ratio of the cationic agent to the therapeutic and/or prophylactic agent is from about 1.5:1 to about 9:1.
Embodiment 73. The loaded LNP of any one of the preceding embodiments, wherein a molar ratio of the cationic agent to the therapeutic and/or prophylactic agent is from about 1.5:1 to about 8:1.
Embodiment 74. The loaded LNP of any one of the preceding embodiments, wherein a molar ratio of the cationic agent to the therapeutic and/or prophylactic agent is from about 1.5:1 to about 7:1.
Embodiment 75. The loaded LNP of any one of the preceding embodiments, wherein a molar ratio of the cationic agent to the therapeutic and/or prophylactic agent is from about 1.5:1 to about 6:1.
Embodiment 76. The loaded LNP of any one of the preceding embodiments, wherein a molar ratio of the cationic agent to the therapeutic and/or prophylactic agent is from about 1.5:1 to about 5:1.
Embodiment 77. The loaded LNP of any one of the preceding embodiments, wherein a molar ratio of the cationic agent to the therapeutic and/or prophylactic agent is about 1.5:1.
Embodiment 78. The loaded LNP of any one of the preceding embodiments, wherein a molar ratio of the cationic agent to the therapeutic and/or prophylactic agent is about 2:1.
Embodiment 79. The loaded LNP of any one of the preceding embodiments, wherein a molar ratio of the cationic agent to the therapeutic and/or prophylactic agent is about 3:1.
Embodiment 80. The loaded LNP of any one of the preceding embodiments, wherein a molar ratio of the cationic agent to the therapeutic and/or prophylactic agent is about 4:1.
Embodiment 81. The loaded LNP of any one of the preceding embodiments, wherein a molar ratio of the cationic agent to the therapeutic and/or prophylactic agent is about 5:1.
Embodiment 82. The empty LNP or loaded LNP of any one of the preceding embodiments, wherein the ionizable lipid is a compound of Formula (TL-A):

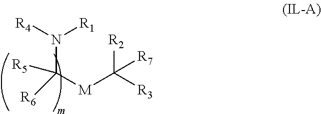

or its N-oxide,
   or a salt or isomer thereof, wherein:
   R¹ is selected from the group consisting of C₅₋₃₀ alkyl, C₅₋₂₀ alkenyl, —R*YR″, —YR″, and —R″M′R′;
   R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
   R⁴ is selected from the group consisting of hydrogen, a C₃₋₆ carbocycle, —(CH₂)ₙQ, —(CH₂)ₙCHQR, —(CH₂)ₒC(R¹²)₂(CH₂)ₙ₋ₒQ, —CHQR, —CQ(R)₂, —C(O)NQR and unsubstituted C₁₋₆ alkyl, where Q is selected from a carbocycle, heterocycle, —OR, —O(CH₂)ₙN(R)₂, —C(O)OR, —OC(O)R, —OC(O)O—, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R′, —N(R)S(O)₂R⁸, —O(CH₂)ₙOR, —N(R)C(═NR₉)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, —(CH₂)ₙN(R)₂, —C(R)N(R)₂C(O)OR, NR^{A}S(O)₂R^{SX}, and

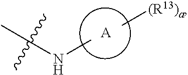

wherein A is a 3-14 membered heterocycle containing one or more heteroatoms selected from N, O and S; and a is 1, 2, 3, or 4; wherein

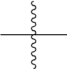

denotes a point of attachment;
each o is independently selected from 1, 2, 3, and 4, and each n is independently selected from 1, 2, 3, 4, and 5;
R′ is selected from the group consisting of C₃₋₆ carbocycle and heterocycle;
R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
R¹² is selected from the group consisting of H, OH, C₁₋₃ alkyl, and C₂₋₃ alkenyl;
each R is independently selected from the group consisting of C₁₋₆ alkyl, C₁₋₃ alkyl-aryl, C₂₋₃ alkenyl, and H;
R^{A} is selected from H and C₁₋₃ alkyl;
R^{SX} is selected from a C₃₋₈ carbocycle, a 3-14 membered heterocycle containing one or more heteroatoms selected from N, O and S, C₁₋₆ alkyl, C₂₋₆ alkenyl, (C₁₋₃ alkoxy)C₁₋₃ alkyl, (CH₂)ₚ₁O(CH₂)ₚ₂R^{SX1}, and (CH₂)ₚ₁R^{SX1}, wherein the carbocycle and heterocycle are optionally substituted with one or more groups selected from oxo, C₁₋₆ alkyl, and (C₁₋₃ alkoxy)C₁₋₃ alkyl;
R^{SX1} is selected from C(O)NR¹⁴R¹⁴′, a C₃₋₈ carbocycle, and a 3-14 membered heterocycle containing one or more heteroatoms selected from N, O and S, wherein the carbocycle and heterocycle are each optionally substituted with one or more groups selected from oxo, halo, C₁₋₃ alkyl, (C₁₋₃ alkoxy)C₁₋₃ alkyl, C₁₋₆ alkylamino, di-(C₁₋₆ alkyl) amino, and NH₂;
each R¹³ is selected from the group consisting of OH, oxo, halo, C₁₋₆ alkyl, C₁₋₆ alkoxy, C₂₋₆ alkenyl, C₁₋₆ alkylamino, di-(C₁₋₆ alkyl) amino, NH₂, C(O)NH₂, CN, and NO₂;
R¹⁴ and R^{14′} are each independently selected from the group consisting of H and C₁₋₆ alkyl;
p₁ is selected from 1, 2, 3, 4, and 5;
p₂ is selected from 1, 2, 3, 4, and 5;
each R⁵ is independently selected from the group consisting of OH, C₁₋₃ alkyl, C₂₋₃ alkenyl, and H;
each R⁶ is independently selected from the group consisting of OH, C₁₋₃ alkyl, C₂₋₃ alkenyl, and H;
R⁷ is selected from the group consisting of C₁₋₃ alkyl, C₂₋₃ alkenyl, and H;
M and M′ are independently selected from —C(O)O—, —OC(O)—, —OC(O)O—, —OC(O)-M″-C(O)O—, —C(O)N(R^{M})—, —N(R^{M})C(O)—, —C(O)—, —C(S)—, —C(S)S—, —SC(S)—, —CH(OH)—, —P(O)(OR^{M})O—, —S(O)₂—, —S—S—, an aryl group, and a heteroaryl group, in which M″ is a bond, C₁₋₃ alkyl or C₂₋₁₃ alkenyl;
each R^{M} is independently selected from the group consisting of H, C₁₋₆ alkyl and C₂₋₆ alkenyl;
each R′ is independently selected from the group consisting of C₁₋₁₈ alkyl, C₂₋₁₈ alkenyl, —R*YR″, —YR″, (CH₂)_{q′}OR*, and H,
   and each q′ is independently selected from 1, 2, and 3;
   each R″ is independently selected from the group consisting of C₃₋₁₅ alkyl and C₃₋₁₅ alkenyl;
   each R* is independently selected from the group consisting of C₁₋₁₂ alkyl and C₂₋₁₂ alkenyl;
   each Y is independently a C₃₋₆ carbocycle;
   each X is independently selected from the group consisting of F, Cl, Br, and I; and
   m is selected from 5, 6, 7, 8, 9, 10, 11, 12, and 13.
Embodiment 83. The empty LNP or loaded LNP of any one of the preceding embodiments, wherein the ionizable lipid is a compound of Formula (IL-B):

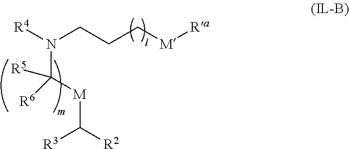

or its N-oxide, or a salt or isomer thereof, wherein R′^{a} is R′^{branched}; wherein
   R′^{branched} is:

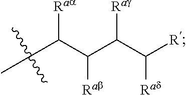

wherein

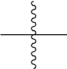

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and
C₂₋₁₄ alkenyl;
R⁴ is selected from the group consisting of —(CH₂)ₙOH, wherein n is selected from the group consisting of 1, 2, 3, 4, and 5, and

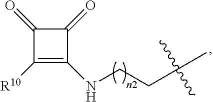

wherein

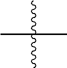

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and
—OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.
Embodiment 84. The empty LNP or loaded LNP of any one of the preceding embodiments, wherein the ionizable lipid is a compound of Formula (TL-C):

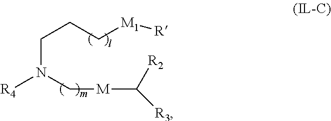

or a salt or isomer thereof, wherein
   l is selected from 1, 2, 3, 4, and 5;
   M₁ is M′;
   R⁴ is —(CH₂)ₙQ, in which Q is OH, and n is selected from 1, 2, 3, 4, or 5;
   M and M′ are independently selected from —C(O)O—, and —OC(O)—;
   R² and R³ are both C₁₋₁₄ alkyl, or C₂₋₁₄ alkenyl; and
   R′ is a C₁-C₁₂ linear alkyl.
Embodiment 85. The empty LNP or loaded LNP of any one of the preceding embodiments, wherein the ionizable lipid is a compound of Formula (IL-D):

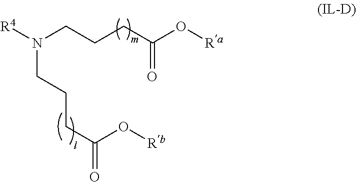

or its N-oxide, or a salt or isomer thereof,
   wherein R′^{a} is R′^{branched} or R′^{cyclic}; wherein
   R′^{branched} is

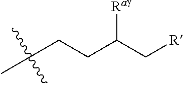

and R′^{b} is

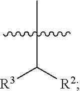

wherein

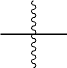

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a CI-12 alkyl or C₂₋₁₂ alkenyl;
m is selected from 1, 2, 3, 4, 5, 6, 7, 8, and 9;
l is selected from 1, 2, 3, 4, 5, 6, 7, 8, and 9.
Embodiment 86. The empty LNP or loaded LNP of any one of the preceding embodiments, wherein the ionizable lipid is a of compound of Formula (IL-I):

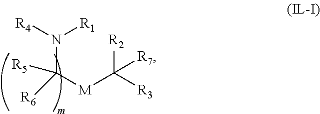

or their N-oxides, or salts or isomers thereof, wherein:
   R¹ is selected from the group consisting of C₅₋₃₀ alkyl, C₅₋₂₀ alkenyl, —R*YR″, —YR″, and —R″M′R′;
   R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
   R⁴ is selected from the group consisting of hydrogen, a C₃₋₆ carbocycle, —(CH₂)ₙQ, —(CH₂)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;
   each R⁵ is independently selected from the group consisting of OH, C₁₋₃ alkyl, C₂₋₃ alkenyl, and H;
   each R⁶ is independently selected from the group consisting of OH, C₁₋₃ alkyl, C₂₋₃ alkenyl, and H;
   M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
   R⁷ is selected from the group consisting of C₁₋₃ alkyl, C₂₋₃ alkenyl, and H;
   R⁸ is selected from the group consisting of C₃₋₆ carbocycle and heterocycle;
   R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
   R¹⁰ is selected from the group consisting of H, OH, C₁₋₃ alkyl, and C₂₋₃ alkenyl;
   each R is independently selected from the group consisting of C₁₋₃ alkyl, C₂₋₃ alkenyl, (CH₂)_{q}OR*, and H,
   and each q is independently selected from 1, 2, and 3;
   each R′ is independently selected from the group consisting of C₁₋₁₈ alkyl, C₂₋₁₈ alkenyl, —R*YR″, —YR″, and H;
   each R″ is independently selected from the group consisting of C₃₋₁₅ alkyl and C₃₋₁₅ alkenyl;
   each R* is independently selected from the group consisting of C₁₋₁₂ alkyl and C₂₋₁₂ alkenyl;
   each Y is independently a C₃₋₆ carbocycle;
   each X is independently selected from the group consisting of F, Cl, Br, and I; and
   m is selected from 5, 6, 7, 8, 9, 10, 11, 12, and 13.
Embodiment 87. The empty LNP or loaded LNP of any one of the preceding embodiments, wherein the ionizable lipid is a compound of Formula (IL-I) wherein R⁷ is H.
Embodiment 88. The empty LNP or loaded LNP of any one of the preceding embodiments, wherein the ionizable lipid is a compound of Formula (IL-I) wherein R⁵ and R⁵ are each H.
Embodiment 89. The empty LNP or loaded LNP of any one of the preceding embodiments, wherein the ionizable lipid is a compound of Formula (IL-I) wherein R¹ is selected from the group consisting of C₅₋₃₀ alkyl, C₅₋₂₀ alkenyl, and —R″M′R′.
Embodiment 90. The empty LNP or loaded LNP of any one of the preceding embodiments, wherein the ionizable lipid is a compound of Formula (IL-I) wherein R¹ is —R″M′R′.
Embodiment 91. The empty LNP or loaded LNP of any one of the preceding embodiments, wherein the ionizable lipid is a of compound of Formula (IL-IA):

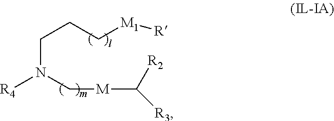

or a salt or isomer thereof, wherein
   l is selected from 1, 2, 3, 4, and 5;
   m is selected from 5, 6, 7, 8, and 9;
   M¹ is a bond or M′;
   R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, or heteroaryl, and each n is selected from 1, 2, 3, 4, or 5;
   M and M′ are independently selected from —C(O)O—, —OC(O)—, —C(O)N(R′)—, —P(O)(OR′)O—, —S—S—, an aryl group, and a heteroaryl group; and
      R₂ and R₃ are both C₁₋₁₄ alkyl or C₂₋₁₄ alkenyl;
   R₈ is selected from the group consisting of C₃₋₆ carbocycle and heterocycle;
   R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
   each R is independently selected from the group consisting of C₁₋₃ alkyl, C₂₋₃ alkenyl, and H; and
   R′ is a C₁₋₁₈ alkyl or C₂₋₁₈ alkenyl.
Embodiment 92. The empty LNP or loaded LNP of any one of the preceding embodiments, wherein the ionizable lipid is a compound of Formula (IL-I) or (IL-IA) wherein R₄ is —(CH₂)ₙQ, in which Q is OH or —N(R)R₈.
Embodiment 93. The empty LNP or loaded LNP of any one of the preceding embodiments, wherein the ionizable lipid is a compound of Formula (IL-IB):

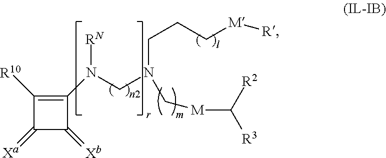

or its N-oxide, or a salt or isomer thereof, wherein
   l is selected from 1, 2, 3, 4, and 5;
   m is selected from 5, 6, 7, 8, and 9;
   R′ is selected from the group consisting of C₁₋₁₄ alkyl and C₂₋₁₄ alkenyl; and
   R₂ and R₃ are independently selected from the group consisting of C₁₋₁₄ alkyl, and C₂₋₁₄ alkenyl;
   M and M′ are independently selected from —C(O)O— and —OC(O)—;
   R_{N} is H, or C₁₋₃ alkyl;
   X^{a} and X^{b} are each independently O or S;
   R₁₀ is selected from the group consisting of H, halo, —OH, R, —N(R)₂, —CN, —N₃, —C(O)OH, —C(O)OR, —OC(O)R, —OR, —SR, —S(O)R, —S(O)OR, —S(O)₂OR, —NO₂, —S(O)₂N(R)₂, —N(R)S(O)₂R, —NH(CH₂)ₜ₁N(R)₂, —NH(CH₂)ₚ₁O(CH₂)_{q1}N(R)₂, —NH(CH₂)ₛ₁OR, —N((CH₂)ₛOR)₂, —N(R)-carbocycle, —N(R)-heterocycle, —N(R)-aryl, —N(R)-heteroaryl, —N(R)(CH₂)ₜ₁-carbocycle, —N(R)(CH₂)ₜ₁-heterocycle, —N(R)(CH₂)ₜ₁-aryl, —N(R)(CH₂)ₜ₁-heteroaryl, a carbocycle, a heterocycle, aryl and heteroaryl;
   each R is independently selected from the group consisting of C₁₋₁₂ alkyl, C₂₋₁₂ alkenyl, and H;
   m is selected from 5, 6, 7, 8, 9, 10, 11, 12, and 13;
   n2 is selected from 1, 2, 3, 4, 5, 6, 7, 8, 9, and 10;
   r is 0 or 1;
   t¹ is selected from 1, 2, 3, 4, and 5;
   p¹ is selected from 1, 2, 3, 4, and 5;
   q¹ is selected from 1, 2, 3, 4, and 5; and
   s¹ is selected from 1, 2, 3, 4.
Embodiment 94. The empty LNP or loaded LNP of any one of the preceding embodiments, wherein the ionizable lipid is a compound of Formula (IL-IB) wherein R^{N} is H.
Embodiment 95. The empty LNP or loaded LNP of any one of the preceding embodiments, wherein the ionizable lipid is a compound of Formula (IL-IB) wherein X^{a} is O.
Embodiment 96. The empty LNP or loaded LNP of any one of the preceding embodiments, wherein the ionizable lipid is a compound of Formula (IL-IB) wherein X^{b} is O.
Embodiment 97. The empty LNP or loaded LNP of any one of the preceding embodiments, wherein the ionizable lipid is a compound of Formula (IL-IB) wherein R¹⁰ is —N(R)₂.
Embodiment 98. The empty LNP or loaded LNP of any one of the preceding embodiments, wherein the ionizable lipid is a compound of Formula (IL-IB) wherein R¹⁰ is —NHCH₃.
Embodiment 99. The empty LNP or loaded LNP of any one of the preceding embodiments, wherein the ionizable lipid is a compound of Formula (IL-IA) or (IL-IB), wherein M₁ is M′.
Embodiment 100. The empty LNP or loaded LNP of any one of the preceding embodiments, wherein the ionizable lipid is a compound of Formula (IL-I), (IL-IA), or (IL-IB), wherein M and M′ are independently —C(O)O— or —OC(O)—.
Embodiment 101. The empty LNP or loaded LNP of any one of the preceding embodiments, wherein the ionizable lipid is a compound of Formula (IL-I), (IL-IA), or (IL-IB), wherein M and M′ are each —C(O)O—.
Embodiment 102. The empty LNP or loaded LNP of any one of the preceding embodiments, wherein the ionizable lipid is a compound of Formula (IL-I), (IL-IA), or (IL-IB), wherein R² and R³ are both C₁₋₁₄ alkyl.
Embodiment 103. The empty LNP or loaded LNP of any one of the preceding embodiments, wherein the ionizable lipid is a compound of Formula (IL-I), (IL-IA), or (IL-IB), wherein R′ is a C₁₋₁₈ alkyl.
Embodiment 104. The empty LNP or loaded LNP of any one of the preceding embodiments, wherein the ionizable lipid is a compound of Formula (IL-I), (IL-1A), or (IL-IB), wherein each R is independently selected from the group consisting of C₁₋₆ alkyl, C₂₋₆ alkenyl, and H.
Embodiment 105. The empty LNP or loaded LNP of any one of the preceding embodiments, wherein the ionizable lipid is a compound of Formula (IL-I), (IL-IA), or (IL-IB), wherein each R is independently selected from the group consisting of C₁₋₆ alkyl, C₂₋₆ alkenyl, and H.
Embodiment 106. The empty LNP or loaded LNP of any one of the preceding embodiments, wherein the ionizable lipid is a compound of Formula (IL-I), (IL-IA), or (IL-IB), wherein each R is independently selected from the group consisting of C₁₋₂ alkyl, C₂ alkenyl, and H.
Embodiment 107. The empty LNP or loaded LNP of any one of the preceding embodiments, wherein the ionizable lipid is a compound of Formula (IL-IC):

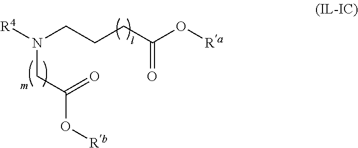

or its N-oxide, or a salt or isomer thereof,
   wherein R′^{a} is R′^{branched} or R′^{cyclic}; wherein
   R′^{branched} is

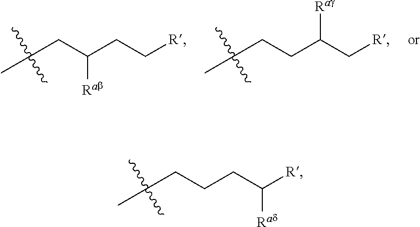

and R′^{cyclic} is:

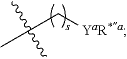

R′^{b} is:

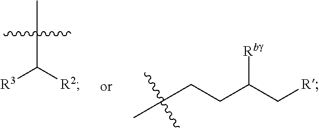

wherein

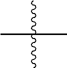

denotes a point of attachment;
wherein R^{aγ}, R^{aγ}, and R^{aγ} are each C₁₋₁₂ alkyl or C₂₋₁₂ alkenyl;
R^{bγ} is H, C₁₋₁₂ alkyl or C₂₋₁₂ alkenyl;
R² and R³ are each independently selected from the group consisting of C₁₋₁₄ alkyl and C₂₋₁₄ alkenyl;
R⁴ is —(CH₂)ₙOH; or

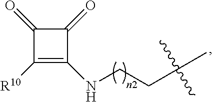

wherein

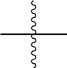

denotes a point of attachment;
each R′ independently is a C₁₋₁₂ alkyl or C₂₋₁₂ alkenyl;
R¹⁰ is N(R)₂; each R is independently selected from the group consisting of C₁₋₆ alkyl, C₂₋₃ alkenyl, and H; and n and n2 are each selected from the group consisting of 1, 2, 3, 4, and 5;
Y^{a} is a C₃₋₆ carbocycle;
R*″^{a} is selected from the group consisting of C₁₋₁₅ alkyl and C₂₋₁₅ alkenyl;
l is selected from 1, 2, 3, 4, and 5;
m is selected from 5, 6, 7, 8, and 9; and
s is 2 or 3.
Embodiment 108. The empty LNP or loaded LNP of any one of the preceding embodiments, wherein the ionizable lipid is a compound of Formula (IL-IC) wherein R^{aγ}, R^{aγ}, and R^{aγ} are each C₁₋₆ alkyl or C₂₋₆ alkenyl.
Embodiment 109. The empty LNP or loaded LNP of any one of the preceding embodiments, wherein the ionizable lipid is a compound of Formula (IL-IC) wherein R^{bγ} is C₁₋₆ alkyl or C₂₋₆ alkenyl.
Embodiment 110. The empty LNP or loaded LNP of any one of the preceding embodiments, wherein the ionizable lipid is a compound of Formula (IL-IC) wherein R′^{branched} is

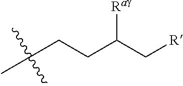

Embodiment 111. The empty LNP or loaded LNP of any one of the preceding embodiments, wherein the ionizable lipid is a compound of Formula (IL-IC) wherein R′^{branched} is

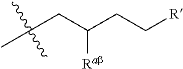

Embodiment 112. The empty LNP or loaded LNP of any one of the preceding embodiments, wherein the ionizable lipid is a compound of Formula (IL-IC) wherein

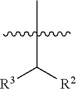

or

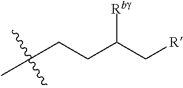

wherein R^{bγ} is C₁₋₆ alkyl;
Embodiment 113. The empty LNP or loaded LNP of any one of the preceding embodiments, wherein the ionizable lipid is a compound of Formula (IL-IB) or (IL-IC) wherein n2 is selected from 2, 3, and 4
Embodiment 114. The empty LNP or loaded LNP of any one of the preceding embodiments, wherein the ionizable lipid is a compound of Formula (IL-IA), (IL-IB), or (IL-IC), wherein 1 is selected from 3, 4, and 5;
Embodiment 115. The empty LNP or loaded LNP of any one of the preceding embodiments, wherein the ionizable lipid is a compound of Formula (IL-I), (IL-IA), (IL-IB), or (IL-IC), wherein m is selected from 6, 7, and 8;
Embodiment 116. The empty LNP or loaded LNP of any one of the preceding embodiments, wherein the ionizable lipid is a compound of Formula (IL-I), (IL-IA), or (IL-IC), wherein n is selected from 2, 3, and 4.
Embodiment 117. The empty LNP or loaded LNP of any one of the preceding embodiments, wherein the ionizable lipid is a compound of Formula (IL-I), (IL-IA), or (IL-IC), wherein R′ is a C₁₋₁₈ alkyl.
Embodiment 118. The empty LNP or loaded LNP of any one of the preceding embodiments, wherein the ionizable lipid is a compound of Formula (IL-I), (IL-IA), or (IL-IC), wherein R′ is a linear C₁₋₁₈ alkyl.
Embodiment 119. The empty LNP or loaded LNP of any one of the preceding embodiments, wherein the ionizable lipid is a compound of Formula (IL-I), (IL-IA), or (IL-IC), wherein R′ is a branched C₁₋₁₈ alkyl.
Embodiment 120. The empty LNP or loaded LNP of any one of the preceding embodiments, wherein the ionizable lipid is a compound selected from Table IL-1 or IL-2.
Embodiment 121. The empty LNP or loaded LNP of any one of the preceding embodiments, wherein the ionizable lipid is a compound of Formula (IL-IIA):

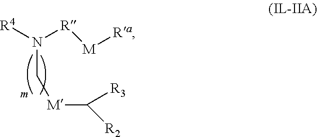

or its N-oxide, or a salt or isomer thereof, wherein:
   m is selected from 5, 6, 7, 8, and 9;
   R² and R³ are each independently selected from the group consisting of H, C₁₋₁₄ alkyl, and C₂₋₁₄ alkenyl;
   R⁴ is selected from —(CH₂)ₙOH, wherein n is selected from 1, 2, 3, 4, and 5, and

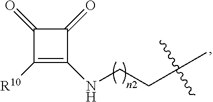

wherein n2 is selected from 1, 2, 3, 4, 5, 6, 7, 8, 9, and 10; and R¹⁰ is —N(R)₂, wherein each R is independently selected from the group consisting of C₁₋₆ alkyl, C₂₋₃ alkenyl, and H;
M is selected from —OC(O)O—, —C(O)O—, —O-M″-O—, and —N(R^{M})C(O)—, in which M″ is —(CH₂)_{z}C(O)—, wherein z is 1, 2, 3, or 4;
M′ is selected from —OC(O)O—, —C(O)O—, —O-M″-O—, —N(R^{M})C(O)O—, and —O—N═C(R^{M})—, wherein:
M″ is —(CH₂)_{z}C(O)—, C₁₋₁₃ alkyl, —B(R**)—, or —Si(R**)₂—;
z is 1, 2, 3, or 4;
each R^{M} is independently selected from H and C₁₋₆ alkyl;
each R** is independently selected from H and C₁₋₁₂ alkyl;
R′^{a} is C₁₋₁₈ alkyl, C₂₋₁₈ alkenyl, or —R*YR*″, wherein:
each R*″ is independently C₁₋₅ alkyl;
each R* is independently C₁₋₁₂ alkyl;
each Y is independently a C₃₋₆ carbocycle; and
R″ is a C₃-C₁₃ alkyl, optionally substituted with OH.
Embodiment 122. The empty LNP or loaded LNP of any one of the preceding embodiments, wherein the ionizable lipid is a compound of Formula (IL-IIA), wherein M is —OC(O)O—.
Embodiment 123. The empty LNP or loaded LNP of any one of the preceding embodiments, wherein the ionizable lipid is a compound of Formula (IL-IIA), wherein M′ is —OC(O)O—.
Embodiment 124. In some embodiments, the ionizable lipid is a compound of Formula (IL-IIAX):

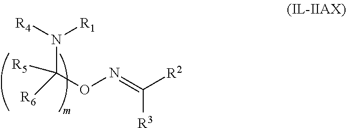

or its N-oxide, or a salt or isomer thereof, wherein:
   R¹ is —R″M′R′, wherein:
   each R′ is independently C₁₋₁₈ alkyl;
   M′ is selected from —C(O)O— and —O—N═C(R^{M})—, wherein each R^{M} is independently selected from H and C₁₋₆ alkyl;
   each R″ is independently C₃₋₁₅ alkyl;
   R² and R³ are each independently selected from the group consisting of H, C₁₋₁₄ alkyl, and C₂₋₁₄ alkenyl;
   R⁴ is selected from —(CH₂)ₙOH, wherein n is selected from 1, 2, 3, 4, and 5, and

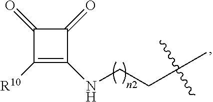

wherein n2 is selected from 1, 2, 3, 4, 5, 6, 7, 8, 9, and 10; and R¹⁰ is —N(R)₂, wherein each R is independently selected from the group consisting of C₁₋₆ alkyl, C₂₋₃ alkenyl, and H;
each R⁵ is H;
each R⁶ is H; and
m is selected from 5, 6, 7, 8, 9, 10, 11, 12, and 13.
Embodiment 125. The empty LNP or loaded LNP of any one of the preceding embodiments, wherein the ionizable lipid is a compound of Formula (IL-IIA) or (IL-IIAX), wherein R⁴ is —(CH₂)ₙOH.
Embodiment 126. The empty LNP or loaded LNP of any one of the preceding embodiments, wherein the ionizable lipid is a compound of Formula (IL-IIA) or (IL-IIAX), wherein R² and R³ are each independently selected from the group consisting of C₁₋₁₄ alkyl, and C₂₋₁₄ alkenyl.
Embodiment 127. The empty LNP or loaded LNP of any one of the preceding embodiments, wherein the ionizable lipid is a compound of Formula (IL-IIA) or (IL-IIAX), wherein R⁴ is

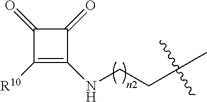

Embodiment 128. The empty LNP or loaded LNP of any one of the preceding embodiments, wherein the ionizable lipid is a compound selected from Table IL-3:
Embodiment 129. The empty LNP or loaded LNP of any one of the preceding embodiments, wherein the ionizable lipid is a compound of Formula (IL-IIB):

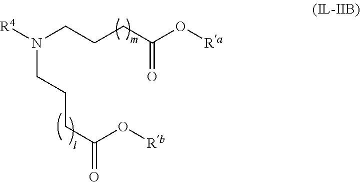

or its N-oxide, or a salt or isomer thereof,
   wherein R′^{a} is

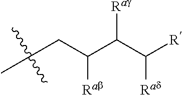

and R′^{b} is

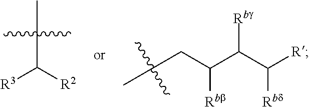

wherein

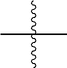

denotes a point of attachment;
R^{aβ}, R^{aγ}, and R^{aδ} are each independently selected from the group consisting of H, C₁₋₁₂ alkyl, and C₂₋₁₂ alkenyl;
R^{bβ}, R^{bγ}, and R^{bδ} are each independently selected from the group consisting of H, C₁₋₁₂ alkyl, and C₂₋₁₂ alkenyl, wherein at least one of RV, R^{bγ}, and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from —(CH₂)ₙNRTQ, —(CH₂)ₙNRS(O)₂TQ, —(CH₂)ₙNRC(O)H and —(CH₂)ₙNRC(O)TQ wherein n is selected from 1, 2, 3, 4, and 5;
T is a bond or a C₁₋₃ alkyl linker, C₂₋₃ alkenyl linker, or C₂₋₃ alkynyl linker;
Q is selected from 3-14 membered heterocycle containing 1-5 heteroatoms selected from N, O, and S, C₃₋₁₀ carbocycle, C₁₋₆ alkyl, and C₂₋₆ alkenyl, wherein the alkyl, alkenyl, heterocycle, and carbocycle are each optionally substituted with one or more R^{Q};
each R^{Q} independently is selected from the group consisting of oxo, hydroxyl, cyano, amino, C₁₋₆ alkylamino, di-C₁₋₆ alkylamino, C₁₋₆ alkyl, C₁₋₆ alkoxy, C₂₋₆ alkenyl, C₁₋₆ alkanolyl, —C(O)C₁₋₆ alkyl, and —NRC(O)C₁₋₆ alkyl;
each R is independently selected from H, C₁₋₆ alkyl, and C₂₋₆ alkenyl;
each R′ is independently selected from C₁₋₁₂ alkyl and C₂₋₁₂ alkenyl;
m is selected from 1, 2, 3, 4, 5, 6, 7, 8, and 9; and
l is selected from 1, 2, 3, 4, 5, 6, 7, 8, and 9.
Embodiment 130. The empty LNP or loaded LNP of any one of the preceding embodiments, wherein the ionizable lipid is a compound of Formula (IL-IIB) wherein R^{aβ} and R^{aδ} are each H.
Embodiment 131. The empty LNP or loaded LNP of any one of the preceding embodiments, wherein the ionizable lipid is a compound of Formula (IL-IIB) wherein R^{aγ} is C₁₋₁₂ alkyl or C₂₋₁₂ alkenyl.
Embodiment 132. The empty LNP or loaded LNP of any one of the preceding embodiments, wherein the ionizable lipid is a compound of Formula (IL-IIB) wherein R^{aγ} is C₁₋₆ alkyl or C₂₋₆ alkenyl.
Embodiment 133. The empty LNP or loaded LNP of any one of the preceding embodiments, wherein the ionizable lipid is a compound of Formula (IL-IIB) wherein R′^{b} is:

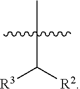

Embodiment 134. The empty LNP or loaded LNP of any one of the preceding embodiments, wherein the ionizable lipid is a compound of Formula (IL-IIB) wherein R′^{b} is

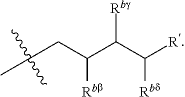

Embodiment 135. The empty LNP or loaded LNP of any one of the preceding embodiments, wherein the ionizable lipid is a compound of Formula (IL-IIB) wherein R^{bβ} and R^{bδ} are each H.
Embodiment 136. The empty LNP or loaded LNP of any one of the preceding embodiments, wherein the ionizable lipid is a compound of Formula (IL-IIB) wherein R^{bγ} is C₁₋₁₂ alkyl or C₂₋₁₂ alkenyl.
Embodiment 137. The empty LNP or loaded LNP of any one of the preceding embodiments, wherein the ionizable lipid is a compound of Formula (IL-IIB) wherein R^{bγ} is C₁₋₆ alkyl or C₂₋₆ alkenyl.
Embodiment 138. The empty LNP or loaded LNP of any one of the preceding embodiments, wherein the ionizable lipid is a compound of Formula (IL-IIB) wherein R⁴ is —(CH₂)ₙNRTQ.
Embodiment 139. The empty LNP or loaded LNP of any one of the preceding embodiments, wherein the ionizable lipid is a compound of Formula (IL-IIB) wherein R⁴ is —(CH₂)ₙNRS(O)₂TQ.
Embodiment 140. The empty LNP or loaded LNP of any one of the preceding embodiments, wherein the ionizable lipid is a compound of Formula (IL-IIB) wherein R⁴ is —(CH₂)ₙNRC(O)H.
Embodiment 141. The empty LNP or loaded LNP of any one of the preceding embodiments, wherein the ionizable lipid is a compound of Formula (IL-IIB) wherein R⁴ is —(CH₂)ₙNRC(O)TQ.
Embodiment 142. The empty LNP or loaded LNP of any one of the preceding embodiments, wherein the ionizable lipid is a compound of Formula (IL-IIB1), (IL-IIB2), (IL-IIB3), or (IL-IIB4):

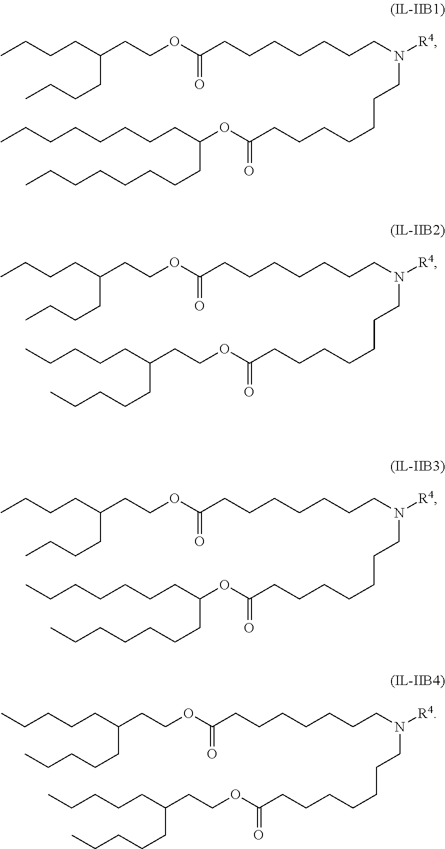

Embodiment 143. The empty LNP or loaded LNP of any one of the preceding embodiments, wherein the ionizable lipid is a compound of Formula (IL-IIB) wherein R⁴ is selected from:

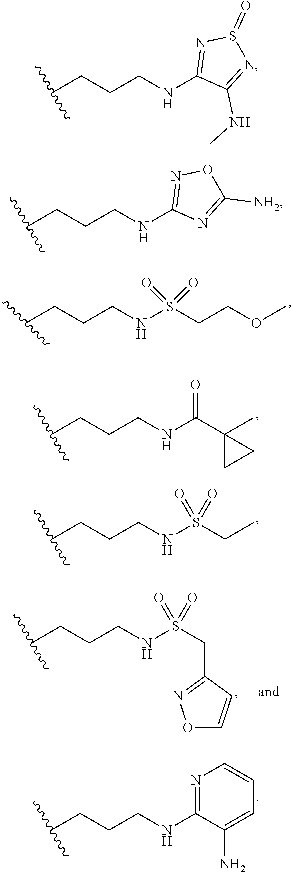

Embodiment 144. The empty LNP or loaded LNP of any one of the preceding embodiments, wherein the ionizable lipid is a compound of Formula (IL-IIB) wherein R⁴ is selected from:

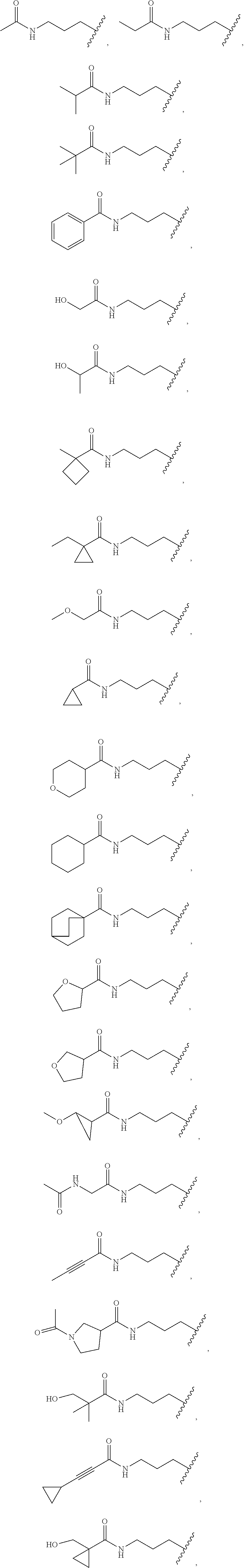

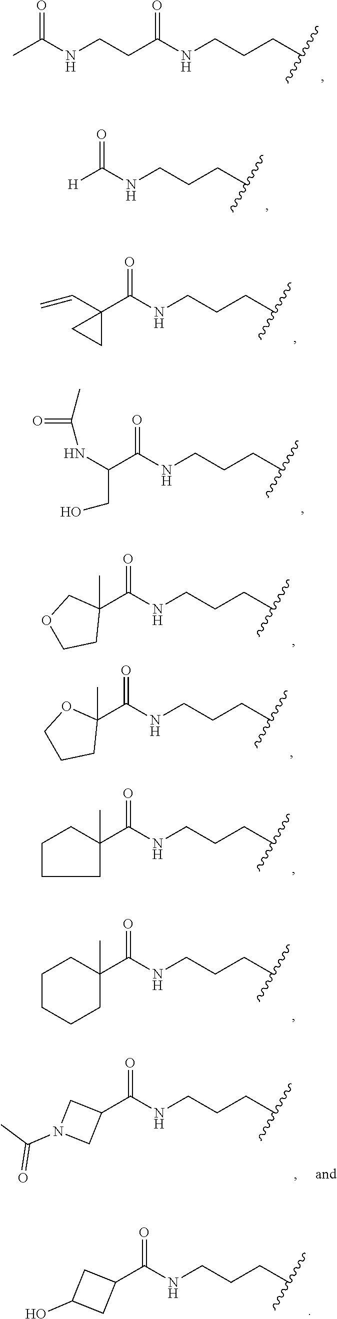

Embodiment 145. The empty LNP or loaded LNP of any one of the preceding embodiments, wherein the ionizable lipid is a compound selected from Table IL-4 or IL-5.
Embodiment 146. The empty LNP or loaded LNP of any one of the preceding embodiments, wherein the ionizable lipid is a compound of Formula (IL-IIC):

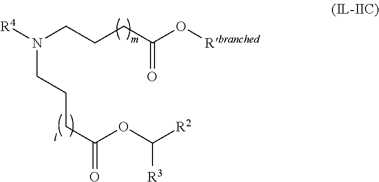

or its N-oxide, or a salt or isomer thereof, wherein:
   R′^{branched} is

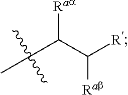

wherein

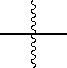

denotes a point of attachment;
wherein R^{aα} and R^{aβ} are each independently selected from the group consisting of H and C₁₋₂ alkyl, wherein at least one of R^{aα} and R^{aβ} is a C₁ or C₂ alkyl;
R′ is selected from the group consisting of C₁₋₁₈ alkyl and C₂₋₁₈ alkenyl;
R² and R³ are each independently selected from the group consisting of C₁₋₁₄ alkyl and C₂₋₁₄ alkenyl;
R⁴ is —(CH₂)ₙQ, wherein n is independently selected from 1, 2, 3, 4, and 5, where Q is selected from NRS(O)₂R^{SX} and

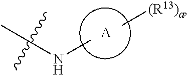

wherein A is a 3-14 membered heterocycle containing one or more heteroatoms selected from N, O and S; and a is 1, 2, 3, or 4; wherein

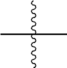

denotes a point of attachment;
R is selected from H and C₁₋₃ alkyl;
R^{SX} is selected from a C₃₋₈ carbocycle, a 3-14 membered heterocycle containing one or more heteroatoms selected from N, O and S, C₁₋₆ alkyl, C₂₋₆ alkenyl, (C₁₋₃ alkoxy)C₁₋₃ alkyl, (CH₂)ₚ₁O(CH₂)ₚ₂R^{SX1}, and (CH₂)ₚ₁R^{SX1}, wherein the carbocycle and heterocycle are optionally substituted with one or more groups selected from oxo, C₁₋₆ alkyl, and (C₁₋₃ alkoxy)C₁₋₃ alkyl;
R^{SX1} is selected from C(O)NR¹⁴R^{14′}, a C₃₋₈ carbocycle, and a 3-14 membered heterocycle containing one or more heteroatoms selected from N, O and S, wherein the carbocycle and heterocycle are each optionally substituted with one or more groups selected from oxo, halo, C₁₋₃ alkyl, (C₁₋₃ alkoxy)C₁₋₃ alkyl, C₁₋₆ alkylamino, di-(C₁₋₆ alkyl) amino, and NH₂;
each R¹³ is selected from the group consisting of OH, oxo, halo, C₁₋₆ alkyl, C₁₋₆ alkoxy, C₂₋₆ alkenyl, C₁₋₆ alkylamino, di-(C₁₋₆ alkyl) amino, NH₂, C(O)NH₂, CN, and NO₂;
R¹⁴ and R^{14′} are each independently selected from the group consisting of H and C₁₋₆ alkyl;
m is selected from 1, 2, 3, 4, 5, 6, 7, 8, and 9;
l is selected from 1, 2, 3, 4, 5, 6, 7, 8, and 9;
p₁ is selected from 1, 2, 3, 4, and 5; and
p₂ is selected from 1, 2, 3, 4.
Embodiment 147. The empty LNP or loaded LNP of any one of the preceding embodiments, wherein the ionizable lipid is a compound of Formula (IL-IIC1) or (IL-IIC2):

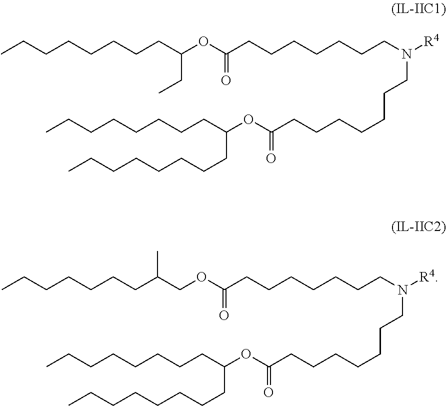

Embodiment 148. The empty LNP or loaded LNP of any one of the preceding embodiments, wherein the ionizable lipid is a compound of Formula (IL-IIC) wherein n is 3.
Embodiment 149. The empty LNP or loaded LNP of any one of the preceding embodiments, wherein the ionizable lipid is a compound of Formula (IL-IIC), (IL-IIC1), or (IL-IIC2), wherein Q is NRS(O)₂R^{SX}.
Embodiment 150. The empty LNP or loaded LNP of any one of the preceding embodiments, wherein the ionizable lipid is a compound of Formula (IL-IIC), (IL-IIC1), or (IL-IIC2), wherein R is H.
Embodiment 151. The empty LNP or loaded LNP of any one of the preceding embodiments, wherein the ionizable lipid is a compound of Formula (IL-IIC), (IL-IIC1), or (IL-IIC2), wherein R^{SX} is selected from a C₃₋₆ carbocycle and a C₁₋₃ alkyl.
Embodiment 152. The empty LNP or loaded LNP of any one of the preceding embodiments, wherein the ionizable lipid is a compound of Formula (IL-IIC), (IL-IIC1), or (IL-IIC2), wherein R^{SX} is ethyl or cyclopropyl.
Embodiment 153. The empty LNP or loaded LNP of any one of the preceding embodiments, wherein the ionizable lipid is a compound of Formula (IL-IIC), (IL-IIC1), or (IL-IIC2), wherein R^{SX} is (CH₂)ₚ₁R^{SX1}.
Embodiment 154. The empty LNP or loaded LNP of any one of the preceding embodiments, wherein the ionizable lipid is a compound of Formula (IL-IIC), (IL-IIC1), or (IL-IIC2), wherein p₁ is 1 and R^{SX1} is a 6-membered heterocycloalkyl, a 5-membered heteroaryl, or phenyl.
Embodiment 155. The empty LNP or loaded LNP of any one of the preceding embodiments, wherein the ionizable lipid is a compound of Formula (IL-IIC), (IL-IIC1), or (IL-IIC2), wherein R^{SX1} is oxazlole or isoxazole.
Embodiment 156. The empty LNP or loaded LNP of any one of the preceding embodiments, wherein the ionizable lipid is a compound of Formula (IL-IIC), (IL-IIC1), or (IL-IIC2), wherein Q is H

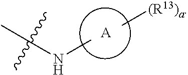

Embodiment 157. The empty LNP or loaded LNP of any one of the preceding embodiments, wherein the ionizable lipid is a compound of Formula (IL-IIC), (IL-IIC1), or (IL-IIC2), wherein A is a 5-membered heteroaryl.
Embodiment 158. The empty LNP or loaded LNP of any one of the preceding embodiments, wherein the ionizable lipid is a compound of Formula (IL-IIC), (IL-IIC1), or (IL-IIC2), wherein each R¹³ is selected from the group consisting of oxo, CI-s alkylamino, di-(C₁₋₆ alkyl) amino, and NH₂.
Embodiment 159. The empty LNP or loaded LNP of any one of the preceding embodiments, wherein the ionizable lipid is a compound of Formula (IL-IIC), (IL-IIC1), or (IL-IIC2), wherein R⁴ is

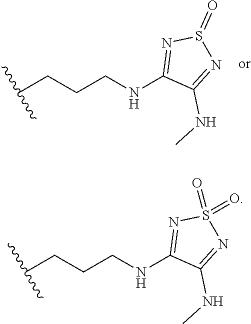

Embodiment 160. The empty LNP or loaded LNP of any one of the preceding embodiments, wherein the ionizable lipid is a compound selected from Table IL-6.
Embodiment 161. The empty LNP or loaded LNP of any one of the preceding embodiments wherein the ionizable lipid is a compound of Formula (IL-III):

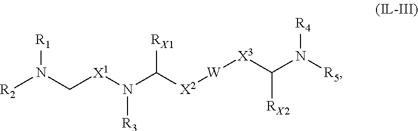

or salts or isomers thereof, wherein,

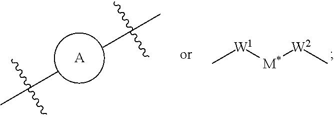

W is

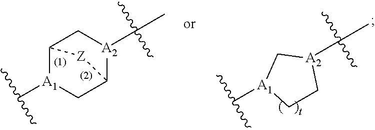

ring A is
t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R¹, R², R³, R⁴, and R⁵ are independently selected from the group consisting of C₅₋₂₀ alkyl, C₅₋₂₀ alkenyl, —R″MR′, —R*YR″, —YR″, and —R*OR″;
R^{X1} and R^{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)— each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6.
Embodiment 162. The empty LNP or loaded LNP of any one of the preceding embodiments, wherein the ionizable lipid is a compound of Formula (IL-III) wherein W is

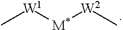

Embodiment 163. The empty LNP or loaded LNP of any one of the preceding embodiments, wherein the ionizable lipid is a compound of Formula (IL-III) wherein W is

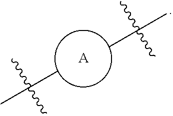

Embodiment 164. The empty LNP or loaded LNP of any one of the preceding embodiments, wherein the ionizable lipid is a compound of Formula (IL-III) wherein W is

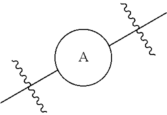

and ring A is

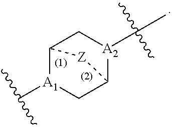

Embodiment 165. The empty LNP or loaded LNP of any one of the preceding embodiments, wherein the ionizable lipid is a compound of Formula (IL-III) wherein W is

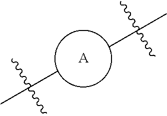

and ring A is

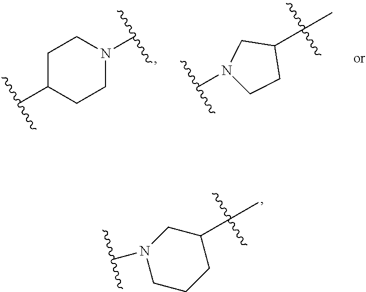 [SMILES: *C1CCN(*)CC1 ; *C1CCN(*)C1 ; *C1CCCN(*)C1]

in which the N atom is connected with X².
Embodiment 166. The empty LNP or loaded LNP of any one of the preceding embodiments, wherein the ionizable lipid is a compound of Formula (IL-III) wherein W is

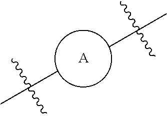

and ring A is

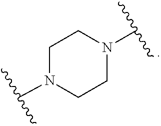 [SMILES: *N1CCN(*)CC1]

Embodiment 167. The empty LNP or loaded LNP of any one of the preceding embodiments, wherein the ionizable lipid is a compound of Formula (IL-IIIA):

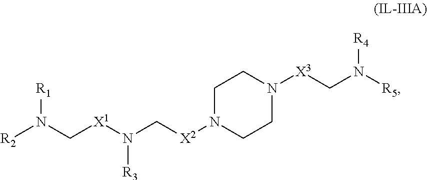

or a salt or isomer thereof, wherein
   R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
   each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
   X¹, X², and X³ are independently selected from the group consisting of a bond, —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
   each Y is independently a C₃₋₆ carbocycle;
   each R* is independently selected from the group consisting of C₁₋₁₂ alkyl and C₂₋₁₂ alkenyl;
   each R is independently selected from the group consisting of C₁₋₃ alkyl and a C₃₋₆ carbocycle;
   each R′ is independently selected from the group consisting of C₁₋₁₂ alkyl, C₂₋₁₂ alkenyl, and H; and
   each R″ is independently selected from the group consisting of C₃₋₁₂ alkyl and C₃₋₁₂ alkenyl.
Embodiment 168. The empty LNP or loaded LNP of any one of the preceding embodiments, wherein the ionizable lipid is a compound of Formula (IL-III) or (IL-IIIA), wherein R₁, R₂, R₃, R₄, and R₅ are each independently selected from C₅₋₂₀ alkyl and C₅₋₂₀ alkenyl.
Embodiment 169. The empty LNP or loaded LNP of any one of the preceding embodiments, wherein the ionizable lipid is a compound of Formula (IL-III) or (IL-IIIA), wherein R₁, R₂, R₃, R₄, and R₅ are each independently C₆ alkyl, C₉ alkyl, C₁₂ alkyl, or C₁₄ alkyl.
Embodiment 170. The empty LNP or loaded LNP of any one of the preceding embodiments, wherein the ionizable lipid is a compound of Formula (IL-III) or (IL-IIIA), wherein X¹ is —CH₂—.
Embodiment 171. The empty LNP or loaded LNP of any one of the preceding embodiments, wherein the ionizable lipid is a compound of Formula (IL-III) or (IL-IIIA), wherein X² is —C(O)—.
Embodiment 172. The empty LNP or loaded LNP of any one of the preceding embodiments, wherein the ionizable lipid is a compound of Formula (IL-III) or (IL-IIIA), wherein X³ is —C(O)—.
Embodiment 173. The empty LNP or loaded LNP of any one of the preceding embodiments, wherein the ionizable lipid is a compound selected from Table IL-7.
Embodiment 174. The empty LNP or loaded LNP of any one of the preceding embodiments, wherein the ionizable lipid is a compound selected from Compounds (I-18), (I-25), (I-301), (II-6), and (VI-4).
Embodiment 175. The empty LNP or loaded LNP of any one of the preceding embodiments, wherein the ionizable lipid is a compound selected from the compounds of Tables IL-1 to IL-7.
Embodiment 176. The empty LNP or loaded LNP of any one of the preceding embodiments, wherein the phospholipid is selected from the group consisting of
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine (18:0 Diether PC),
1-oleoyl-2-cholesterylhemisuccinoyl-sn-glycero-3-phosphocholine (OChemsPC),
1-hexadecyl-sn-glycero-3-phosphocholine (C16 Lyso PC),
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-dioleoyl-sn-glycero-3-phosphoethanolamine (DOPE),
1,2-diphytanoyl-sn-glycero-3-phosphoethanolamine (ME 16.0 PE),
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG),
dipalmitoylphosphatidylglycerol (DPPG), palmitoyloleoylphosphatidylethanolamine (POPE),
distearoyl-phosphatidyl-ethanolamine (DSPE),
dipalmitoyl phosphatidyl ethanolamine (DPPE),
dimyristoylphosphoethanolamine (DMPE),
1-stearoyl-2-oleoyl-phosphatidyethanolamine (SOPE),
1-stearoyl-2-oleoyl-phosphatidylcholine (SOPC),
sphingomyelin, phosphatidylcholine, phosphatidylethanolamine,
phosphatidylserine,
phosphatidylinositol,
phosphatidic acid,
palmitoyloleoyl phosphatidylcholine,
lysophosphatidylcholine,
lysophosphatidylethanolamine (LPE)
sphingomyelin, and
   mixtures thereof.
   Embodiment 177. The empty LNP or loaded LNP of any one of the preceding embodiments, wherein the phospholipid is DSPC.
   Embodiment 178. The empty LNP or loaded LNP of any one of the preceding embodiments, wherein the structural lipid is selected from the group consisting of cholesterol, fecosterol, sitosterol, ergosterol, campesterol, stigmasterol, brassicasterol, tomatidine, ursolic acid, alpha-tocopherol, and mixtures thereof.
   Embodiment 179. The empty LNP or loaded LNP of any one of the preceding embodiments, wherein the structural lipid is

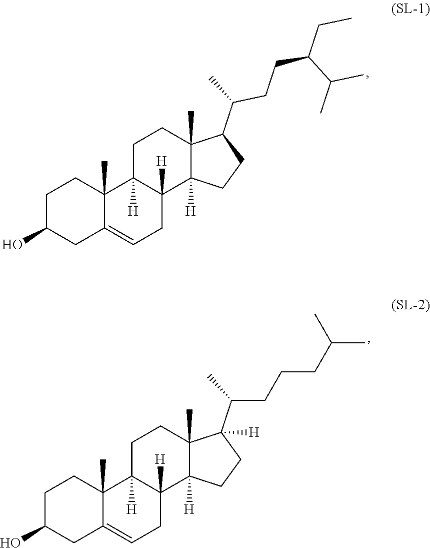

or a salt thereof.
Embodiment 180. The empty LNP or loaded LNP of any one of the preceding embodiments, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof
Embodiment 181. The empty LNP or loaded LNP of any one of the preceding embodiments, wherein the PEG lipid is a compound of one of the following structures:

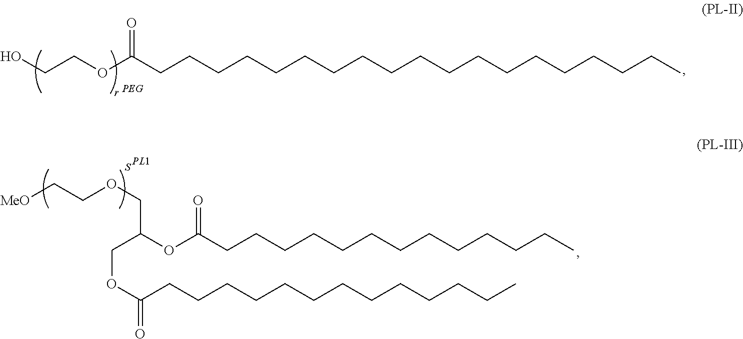

or a salt thereof, wherein r is an integer from 1 to 100; and s is an integer from 1 to 100.
Embodiment 182. The empty LNP or loaded LNP of any one of the preceding embodiments, wherein the PEG lipid is one of the following compounds:

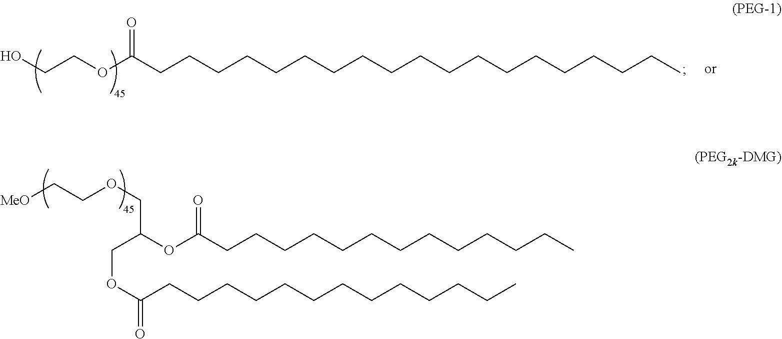

or a salt thereof.
Embodiment 183. The loaded LNP of any one of the preceding embodiments, wherein the therapeutic and/or prophylactic agent is a nucleic acid.
Embodiment 184. The loaded LNP of any one of the preceding embodiments, wherein the therapeutic and/or prophylactic agent is a ribonucleic acid (RNA).
Embodiment 185. The loaded LNP of any one of the preceding embodiments, wherein the RNA is selected from the group consisting of a short interfering RNA (siRNA), an asymmetrical interfering RNA (aiRNA), a RNA interference (RNAi) molecule, a microRNA (miRNA), an antagomir, an antisense RNA, a ribozyme, a Dicer substrate RNA (dsRNA), a small hairpin RNA (shRNA), a messenger RNA (mRNA), and mixtures thereof.
Embodiment 186. The loaded LNP of any one of the preceding embodiments, wherein the RNA is mRNA.
Embodiment 187. The loaded LNP of any one of the preceding embodiments, wherein the mRNA is a modified mRNA (mmRNA).
Embodiment 188. The loaded LNP of any one of the preceding embodiments, wherein the mRNA incorporates a micro-RNA binding site (miR binding site).
Embodiment 189. The loaded LNP of any one of the preceding embodiments, wherein the mRNA includes one or more of a stem loop, a chain terminating nucleoside, a polyA sequence, a polyadenylation signal, and/or a 5′ cap structure.
Embodiment 190. A pharmaceutical composition comprising the loaded LNP of any one of the preceding embodiments and a pharmaceutically acceptable carrier.
Embodiment 191. A method of delivering a therapeutic and/or prophylactic agent to a cell within a subject, the method comprising administering to the subject the loaded LNP of any one of the preceding embodiments.
Embodiment 192. A method of producing a polypeptide of interest in a cell within a subject, the method comprising administering to the subject the loaded LNP of any one of the preceding embodiments.
Embodiment 193. The loaded LNP of any one of the preceding embodiments, for use in a method of delivering a therapeutic and/or prophylactic agent to a cell within a subject, the method comprising administering to the subject said loaded LNP.
Embodiment 194. The loaded LNP of any one of the preceding embodiments, for use in a method of producing a polypeptide of interest in a cell within a subject, the method comprising administering to the subject said loaded LNP.
Embodiment 195. The method or loaded LNP for use of any one of the preceding embodiments, wherein the cell is an endothelial cell.
Embodiment 196. The method or loaded LNP for use of any one of the preceding embodiments, wherein the endothelial cell is a pulmonary endothelial cell.
Embodiment 197. The method or loaded LNP for use of any one of the preceding embodiments, wherein the endothelial cell is a respiratory endothelial cell.
Embodiment 198. The method or loaded LNP for use of any one of the preceding embodiments, wherein the endothelial cell is a bronchial endothelial cell.
Embodiment 199. A method of specifically delivering a therapeutic and/or prophylactic agent to an organ of a subject, the method comprising administering to the subject the loaded LNP of any one of any one of the preceding embodiments.
Embodiment 200. The loaded LNP of any one of the preceding embodiments, for use in a method of specifically delivering a therapeutic and/or prophylactic agent to an organ of a subject, the method comprising administering to the subject said loaded LNP.
Embodiment 201. The method or loaded LNP for use of any one of the preceding embodiments, wherein the organ is selected from the group consisting of liver, kidney, lung, and spleen.
Embodiment 202. The method or loaded LNP for use of any one of the preceding embodiments, wherein the organ is the lung.
Embodiment 203. A method of specifically delivering a therapeutic and/or prophylactic agent to a tissue of a subject, the method comprising administering to the subject the loaded LNP of any one of the preceding embodiments.
Embodiment 204. The loaded LNP of any one of the preceding embodiments, for use in a method of specifically delivering a therapeutic and/or prophylactic agent to a tissue of a subject, the method comprising administering to the subject said loaded LNP.
Embodiment 205. The method or loaded LNP for use of any one of the preceding embodiments, wherein the tissue is the endothelium.
Embodiment 206. The method or loaded LNP for use of any one of the preceding embodiments, wherein the tissue is the pulmonary endothelium.
Embodiment 207. A method of treating a disease or disorder in a subject in need thereof, the method comprising administering to the subject the loaded LNP of any one of the preceding embodiments.
Embodiment 208. The loaded LNP of any one of the preceding embodiments, for use in a method of treating a disease or disorder in a subject in need thereof, the method comprising administering to the subject said loaded LNP.
Embodiment 209. The method or loaded LNP for use of any one of the preceding embodiments, wherein the administering is performed parenterally, intramuscularly, intradermally, subcutaneously, and/or intravenously.

### EQUIVALENTS

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

1. A cationic lipid of Formula (I): 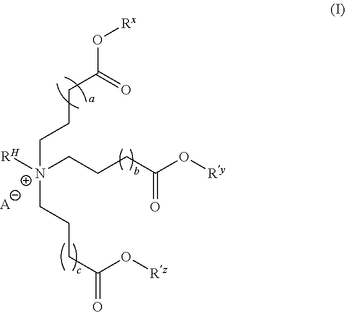
or an isomer thereof, wherein:
R′x is: 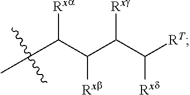
R′^{y} is: 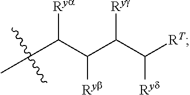
and R′^{z} is: 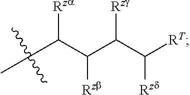
wherein 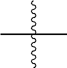
denotes a point of attachment;
R^{xα}, R^{xγ}, R^{xγ}, and R^{xδ} are each independently selected from the group consisting of H, C₁₋₁₂ alkyl, and C₂₋₁₂ alkenyl;
R^{yα}, R^{yβ}, R^{yγ}, and R^{yδ} are each independently selected from the group consisting of H, C₁₋₁₂ alkyl, and C₂₋₁₂ alkenyl;
R^{zα}, R^{zβ}, R^{zγ}, and R^{zδ} are each independently selected from the group consisting of H, C₁₋₁₂ alkyl, and C₂₋₁₂ alkenyl;
R^{H} is —(CH₂)_{q}OH, wherein q is selected from 1, 2, 3, 4, and 5;
each R^{T} is independently selected from C₁₋₁₂ alkyl and C₂₋₁₂ alkenyl;
a is selected from 1, 2, 3, 4, 5, 6, 7, 8, and 9;
b is selected from 1, 2, 3, 4, 5, 6, 7, 8, and 9;
c is selected from 1, 2, 3, 4, 5, 6, 7, 8, and 9; and
A⁻ is any pharmaceutically acceptable anion.

2. The cationic lipid of claim 1, wherein
R′^{x} is 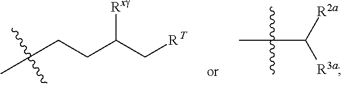
R′^{y} is: 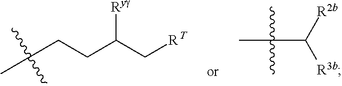
and
R′^{z} is: 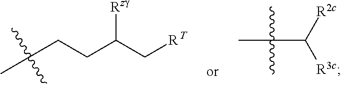
wherein 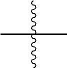
denotes a point of attachment;
R^{xγ} is selected from the group consisting of H, C₁₋₁₂ alkyl, and C₂₋₁₂ alkenyl;
R^{yγ} is selected from the group consisting of H, C₁₋₁₂ alkyl, and C₂₋₁₂ alkenyl;
R^{zγ} is selected from the group consisting of H, C₁₋₁₂ alkyl, and C₂₋₁₂ alkenyl; and
R^{2a}, R^{2b}, R^{2c}, R^{3a}, R^{3b}, and R^{3c} are each independently selected from the group consisting of C₁₋₁₄ alkyl and C₂₋₁₄ alkenyl.

3. The cationic lipid of claim 1, wherein
R′^{a} is: 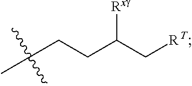
R′^{b} is: 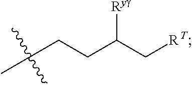
and R′^{c} is: 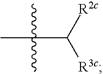
wherein 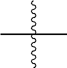
denotes a point of attachment;
R′^{xγ} is selected from the group consisting of H, C₁₋₁₂ alkyl, and C₂₋₁₂ alkenyl;
R^{yγ} is selected from the group consisting of H, C₁₋₁₂ alkyl, and C₂₋₁₂ alkenyl; and
R^{2c} and R^{3c} are each independently selected from the group consisting of C₁₋₁₄ alkyl and C₂₋₁₄ alkenyl.

4. The compound of any one of claims 1-3, wherein R^{xγ} and R^{yγ} are each H.

5. The compound of any one of claims 1-3, wherein R^{xγ} and R^{yγ} are each C₁₋₁₂ alkyl or C₂-12 alkenyl.

6. The compound of any one of claims 1-3, wherein R⁷ is C₁₋₁₂ alkyl or C₂₋₁₂ alkenyl and R^{yγ} is H.

7. The compound of any one of the preceding claims, wherein q is 2.

8. The compound of any one of claims 1-3, wherein the compound is selected from:

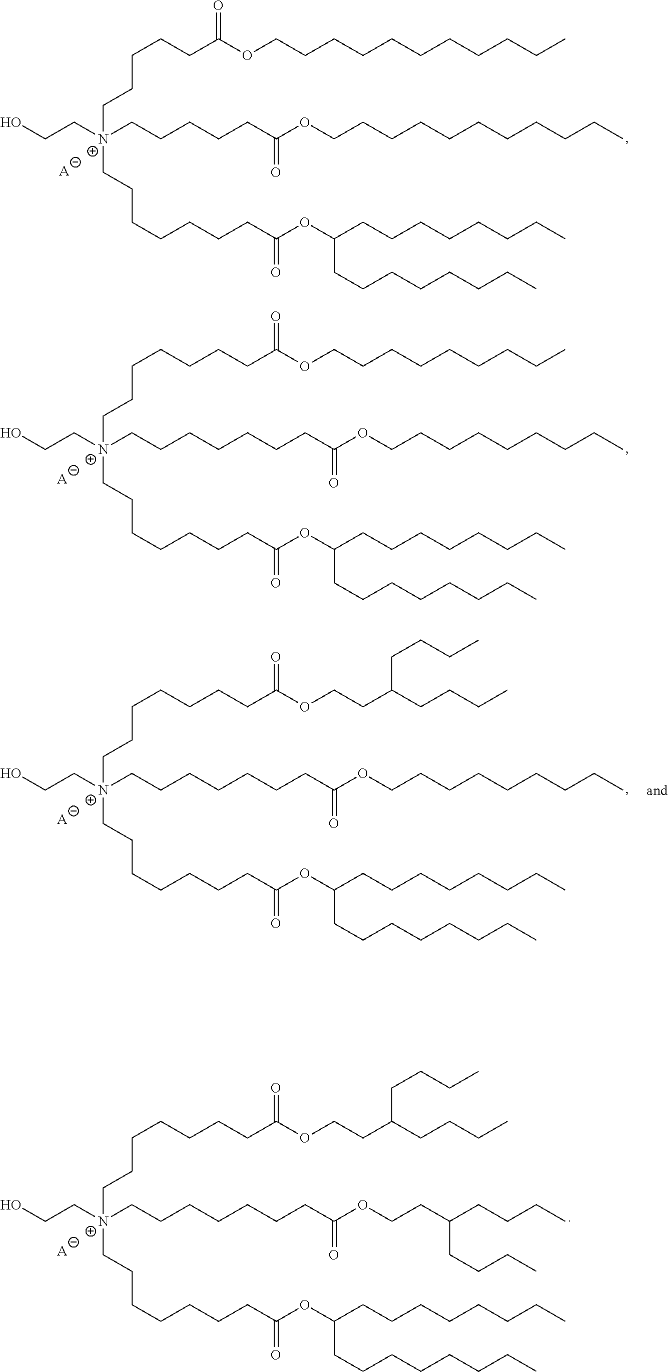

9. The compound of any one of the preceding claims, wherein A⁻ is selected from chloride, bromide, iodide, hydroxide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate.

10. The compound of any one of the preceding claims, wherein A⁻ is selected from bromide, chloride, and hydroxide.

11. An empty lipid nanoparticle (empty LNP) comprising a cationic lipid of any one of the preceding claims.

12. The empty LNP of claim 11, wherein the empty LNP further comprises an ionizable lipid.

13. The empty LNP of claim 11 or 12, wherein the empty LNP further comprises a phospholipid.

14. The empty LNP of any one of claims 11-13, wherein the empty LNP further comprises a structural lipid.

15. The empty LNP of any one of claims 11-14, wherein the empty LNP further comprises a PEG lipid.

16. A empty LNP comprising a lipid component which comprises from about 20 mol % to about 40 mol % of the compound of any one of claims 1-10; from about 15 mol % to about 40 mol % ionizable lipid, from about 0 mol % to about 30 mol % phospholipid, from about 15 mol % to about 50 mol % structural lipid, and from about 0 mol % to about 1 mol % PEG lipid.

17. A loaded lipid nanoparticle (loaded LNP) comprising the empty LNP of any one of claims 11-16 and a therapeutic and/or prophylactic agent.

18. A loaded LNP comprising:
(a) a loaded LNP core comprising:
(i) an ionizable lipid,
(ii) a phospholipid,
(iii) a structural lipid, and
(iv) a PEG-lipid, and
(b) a therapeutic and/or prophylactic agent encapsulated within the core for delivery into a cell, and
(c) a cationic agent, wherein the cationic agent is a cationic lipid of any one of claims 1-10.

19. A loaded LNP comprising:
(a) a loaded LNP core comprising:
(i) an ionizable lipid,
(ii) a phospholipid,
(iii) a structural lipid, and
(iv) a PEG-lipid, and
(b) a therapeutic and/or prophylactic agent encapsulated within the core for delivery into a cell, and
(c) a cationic agent.

20. The loaded LNP of claim 19, wherein the cationic agent is a cationic lipid.

21. The loaded LNP of any one of claims 17-20, wherein the therapeutic and/or prophylactic agent is a nucleic acid.

22. The loaded LNP of claim 21, wherein the therapeutic and/or prophylactic agent is a ribonucleic acid (RNA).

23. The loaded LNP of claim 22, wherein the RNA is selected from the group consisting of a short interfering RNA (siRNA), an asymmetrical interfering RNA (aiRNA), a RNA interference (RNAi) molecule, a microRNA (miRNA), an antagomir, an antisense RNA, a ribozyme, a Dicer substrate RNA (dsRNA), a small hairpin RNA (shRNA), a messenger RNA (mRNA), and mixtures thereof.

24. The loaded LNP of claim 23, wherein the RNA is an mRNA.

25. The loaded LNP of claim 24, wherein the mRNA is a modified mRNA (mmRNA).

26. The loaded LNP of claim 24 or 25, wherein the mRNA incorporates a micro-RNA binding site (miR binding site).

27. The loaded LNP of any one of claims 24-26, wherein the mRNA includes one or more of a stem loop, a chain terminating nucleoside, a polyA sequence, a polyadenylation signal, and/or a 5′ cap structure.

28. The empty LNP or loaded LNP of any one of claims 12-27, wherein the ionizable lipid is a compound of Formula (IL-A): 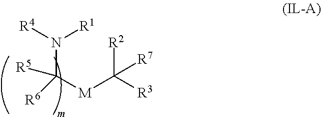
or its N-oxide, or a salt or isomer thereof, wherein:
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of hydrogen, a C₃₋₆ carbocycle, —(CH₂)ₙQ, —(CH₂)ₙCHQR, —(CH₂)ₒC(R¹²)₂(CH₂)ₙ₋ₒQ, —CHQR, —CQ(R)₂, —C(O)NQR and unsubstituted C₁₋₆ alkyl, where Q is selected from a carbocycle, heterocycle, —OR, —O(CH₂)ₙN(R)₂, —C(O)OR, —OC(O)R, —OC(O)O—, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R′, —N(R)S(O)₂R⁸, —O(CH₂)ₙOR, —N(R)C(═NR₉)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, —(CH₂)ₙN(R)₂ and —C(R)N(R)₂C(O)OR, NR^{A}S(O)₂R^{SX}, and 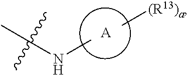
wherein A is a 3-14 membered heterocycle containing one or more heteroatoms selected from N, O and S; and a is 1, 2, 3, or 4; wherein 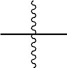
denotes a point of attachment;
each o is independently selected from 1, 2, 3, and 4, and each n is independently selected from 1, 2, 3, 4, and 5;
R⁸ is selected from the group consisting of C₃₋₆ carbocycle and heterocycle;
R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
R¹² is selected from the group consisting of H, OH, C₁₋₃ alkyl, and C₂₋₃ alkenyl;
each R is independently selected from the group consisting of C₁₋₆ alkyl, C₁₋₃ alkyl-aryl, C₂₋₃ alkenyl, and H;
R^{A} is selected from H and C₁₋₃ alkyl;
R^{SX} is selected from a C₃₋₈ carbocycle, a 3-14 membered heterocycle containing one or more heteroatoms selected from N, O and S, C₁₋₆ alkyl, C₂₋₆ alkenyl, (C₁₋₃ alkoxy)C₁₋₃ alkyl, (CH₂)ₚ₁O(CH₂)ₚ₂R^{SX1}, and (CH₂)ₚ₁R^{SX1}, wherein the carbocycle and heterocycle are optionally substituted with one or more groups selected from oxo, C₁₋₆ alkyl, and (C₁₋₃ alkoxy)C₁₋₃ alkyl;
R^{SX1} is selected from C(O)NR¹⁴R^{14′}, a C₃₋₈ carbocycle, and a 3-14 membered heterocycle containing one or more heteroatoms selected from N, O and S, wherein the carbocycle and heterocycle are each optionally substituted with one or more groups selected from oxo, halo, C₁₋₃ alkyl, (C₁₋₃ alkoxy)C₁₋₃ alkyl, C₁₋₆ alkylamino, di-(C₁₋₆ alkyl) amino, and NH₂;
each R¹³ is selected from the group consisting of OH, oxo, halo, C₁₋₆ alkyl, C₁₋₆ alkoxy, C₂₋₆ alkenyl, C₁₋₆ alkylamino, di-(C₁₋₆ alkyl) amino, NH₂, C(O)NH₂, CN, and NO₂;
R¹⁴ and R^{14′} are each independently selected from the group consisting of H and C₁₋₆ alkyl;
p₁ is selected from 1, 2, 3, 4, and 5;
p₂ is selected from 1, 2, 3, 4, and 5;
each R⁵ is independently selected from the group consisting of OH, C₁₋₃ alkyl, C₂₋₃ alkenyl, and H;
each R⁶ is independently selected from the group consisting of OH, C₁₋₃ alkyl, C₂₋₃ alkenyl, and H;
R⁷ is selected from the group consisting of C₁₋₃ alkyl, C₂₋₃ alkenyl, and H;
M and M′ are independently selected from —C(O)O—, —OC(O)—, —OC(O)O—, —OC(O)-M″-C(O)O—, —C(O)N(R^{M})—, —N(R^{M})C(O)—, —C(O)—, —C(S)—, —C(S)S—, —SC(S)—, —CH(OH)—, —P(O)(O R^{M})O—, —S(O)₂—, —S—S—, an aryl group, and a heteroaryl group, in which M″ is a bond, C₁₋₁₃ alkyl or C₂₋₁₃ alkenyl;
each R^{M} is independently selected from the group consisting of H, C₁₋₆ alkyl and C₂₋₆ alkenyl;
each R′ is independently selected from the group consisting of C₁₋₁₈ alkyl, C₂₋₁₈ alkenyl, —R*YR″, —YR″, (CH₂)_{q′}OR*, and H,
and each q′ is independently selected from 1, 2, and 3;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

29. The empty LNP or loaded LNP of claim 28, wherein the ionizable lipid is a compound of Formula (IL-B): 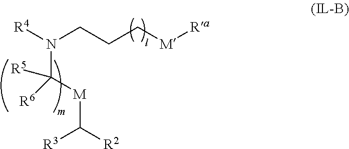
or its N-oxide, or a salt or isomer thereof, wherein:
R′^{a} is R′^{branched}; wherein R′^{branched} is: 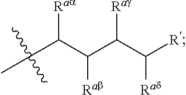
wherein 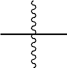
denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and 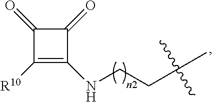
wherein 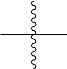
denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

30. The empty LNP or loaded LNP of claim 28, wherein the ionizable lipid is a compound of Formula (TL-C): 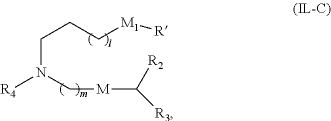
or a salt or isomer thereof, wherein
l is selected from 1, 2, 3, 4, and 5;
M₁ is M′;
R⁴ is —(CH₂)ₙQ, in which Q is OH, and n is selected from 1, 2, 3, 4, or 5;
M and M′ are independently selected from —C(O)O—, and —OC(O)—;
R² and R³ are both C₁₋₁₄ alkyl, or C₂₋₁₄ alkenyl; and
R′ is a C₁-C₁₂ linear alkyl.

31. The empty LNP or loaded LNP of claim 28, wherein the ionizable lipid is a compound of Formula (IL-D): 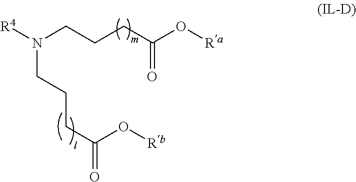
or its N-oxide, or a salt or isomer thereof,
wherein R′^{a} is R′^{branched} or R′^{cyclic}; wherein
R′^{branched} is 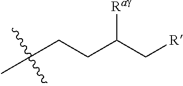
and R′b is: 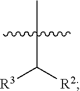
wherein 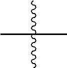
denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

32. The empty LNP or loaded LNP of any one of claims 12-27, wherein the ionizable lipid is a compound of Formula (IL-III): 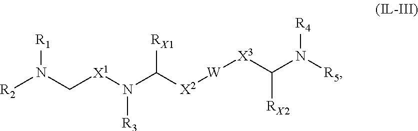
or a salt or isomers thereof, wherein,
W is 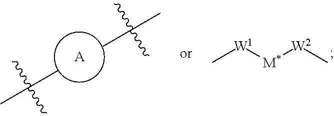
ring A is 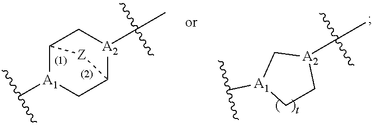
t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R¹, R², R³, R⁴, and R⁵ are independently selected from the group consisting of C₅₋₂₀ alkyl, C₅₋₂₀ alkenyl, —R″MR′, —R*YR″, —YR″, and —R*OR″;
R^{X1} and R^{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R⁶)—;
each R⁶ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6.

33. The empty LNP or loaded LNP of claim 32, wherein the ionizable lipid is a compound of Formula (IL-IIIA): 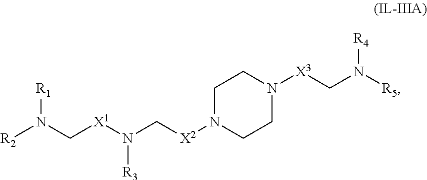
or a salt or isomer thereof, wherein
R¹, R², R³, R⁴, and R⁵ are independently selected from the group consisting of C₅₋₂₀ alkyl, C₅₋₂₀ alkenyl, —R″MR′, —R*YR″, —YR″, and —R*OR″;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
X¹, X², and X³ are independently selected from the group consisting of a bond, —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H; and
each R″ is independently selected from the group consisting of C₃₋₁₂ alkyl and C₃₋₁₂ alkenyl.

34. The empty LNP or loaded LNP of claim 32 or 33, wherein R¹, R², R³, R⁴, and R⁵ are each C₅₋₂₀ alkyl; X¹ is —CH₂—; and X² and X³ are each —C(O)—.

35. The empty LNP or loaded LNP of any one of claims 12-27, wherein the ionizable lipid is a compound selected from:

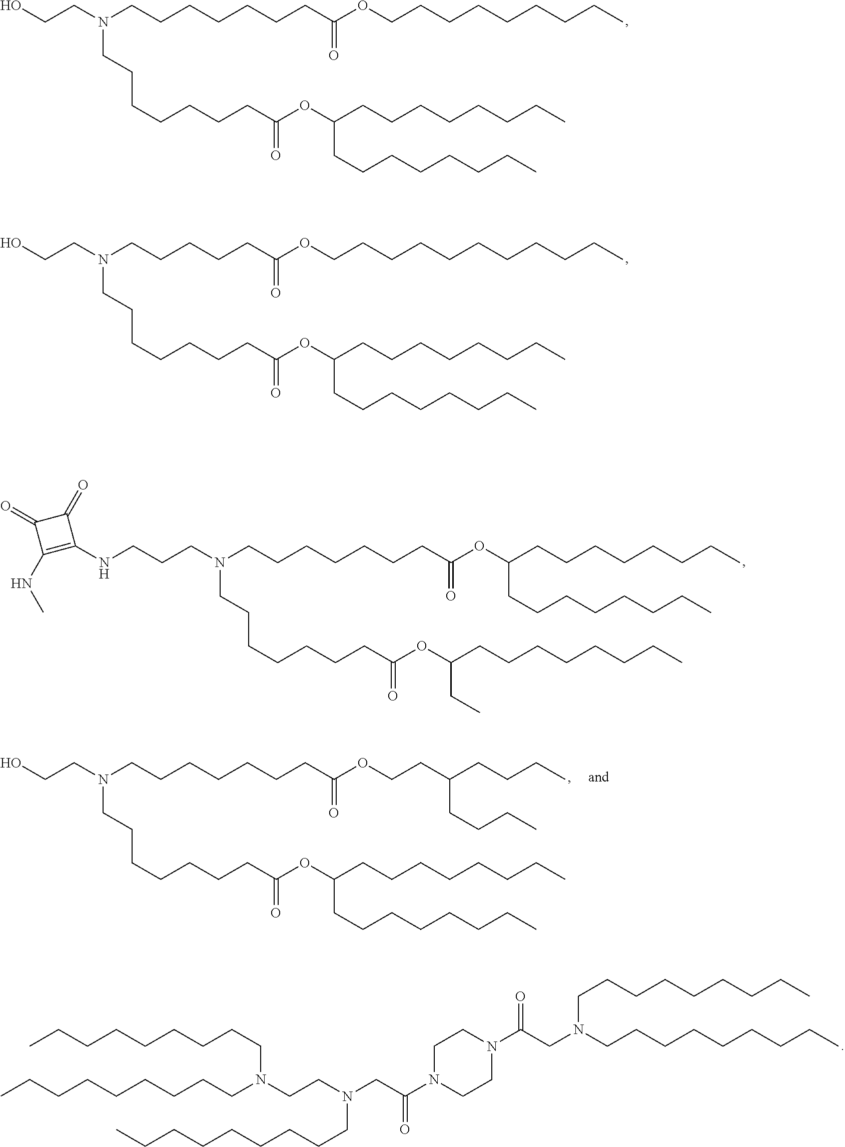

36. The empty LNP or loaded LNP of any one of claims 12-27, wherein the ionizable lipid is a compound selected from the compounds of Tables IL-1 to TL-7.

37. The empty LNP or loaded LNP of any one of claims 13-36, wherein the phospholipid is selected from the group consisting of
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine (18:0 Diether PC),
1-oleoyl-2-cholesterylhemisuccinoyl-sn-glycero-3-phosphocholine (OChemsPC),
1-hexadecyl-sn-glycero-3-phosphocholine (C16 Lyso PC),
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-dioleoyl-sn-glycero-3-phosphoethanolamine (DOPE),
1,2-diphytanoyl-sn-glycero-3-phosphoethanolamine (ME 16.0 PE),
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG),
dipalmitoylphosphatidylglycerol (DPPG), palmitoyloleoylphosphatidylethanolamine (POPE),
distearoyl-phosphatidyl-ethanolamine (DSPE),
dipalmitoyl phosphatidyl ethanolamine (DPPE),
dimyristoylphosphoethanolamine (DMPE),
1-stearoyl-2-oleoyl-phosphatidyethanolamine (SOPE),
1-stearoyl-2-oleoyl-phosphatidylcholine (SOPC),
sphingomyelin, phosphatidylcholine, phosphatidylethanolamine,
phosphatidylserine,
phosphatidylinositol,
phosphatidic acid,
palmitoyloleoyl phosphatidylcholine,
lysophosphatidylcholine,
lysophosphatidylethanolamine (LPE)
sphingomyelin, and
mixtures thereof.

38. The empty LNP or loaded LNP of claim 37, wherein the phospholipid is DSPC.

39. The empty LNP or loaded LNP of any one of claims 14-38, wherein the structural lipid is selected from the group consisting of cholesterol, fecosterol, sitosterol, ergosterol, campesterol, stigmasterol, brassicasterol, tomatidine, ursolic acid, alpha-tocopherol, and mixtures thereof.

40. The empty LNP or loaded LNP of claim 39, wherein the structural lipid is 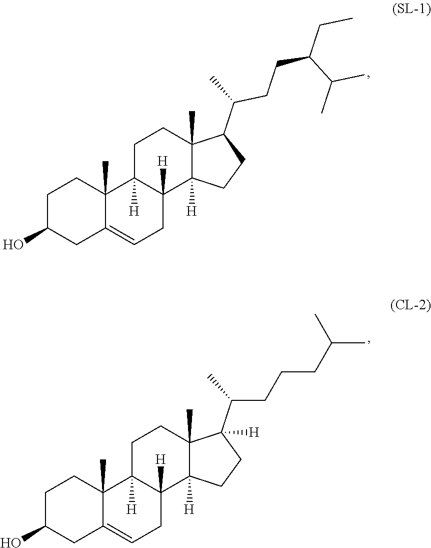
or a salt thereof.

41. The empty LNP or loaded LNP of any one of claims 15-40, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

42. The empty LNP or loaded LNP of claim 41, wherein the PEG lipid is a compound of one of the following structures: 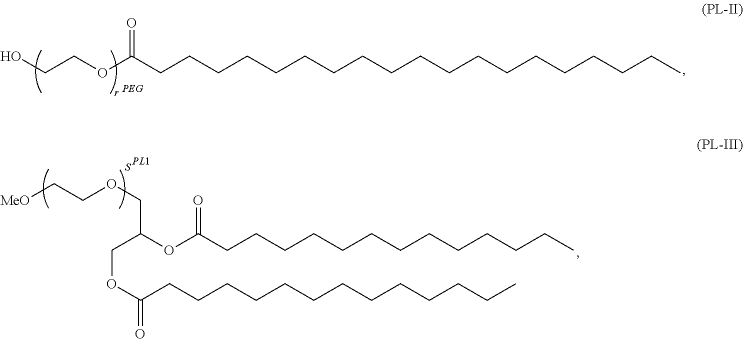
or a salt thereof, wherein r is an integer from 1 to 100; and s is an integer from 1 to 100.

43. The empty LNP or loaded LNP of claim 42, wherein the PEG lipid is one of the following compounds: 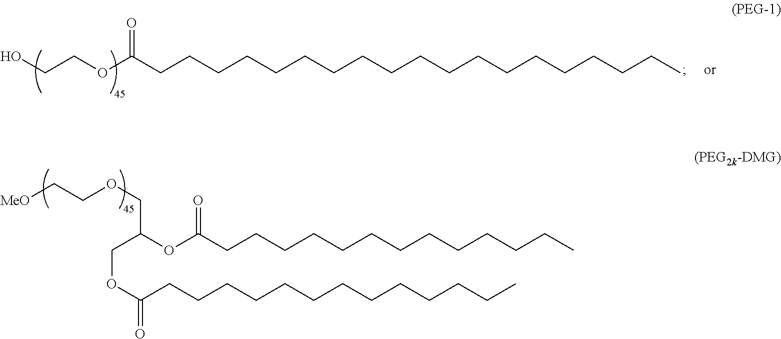
or a salt thereof.

44. A pharmaceutical composition comprising the loaded LNP of any one of claims 17-43 and a pharmaceutically acceptable carrier.

45. A method of delivering a therapeutic and/or prophylactic agent to a cell within a subject, the method comprising administering to the subject the loaded LNP of any one of claims 17-43, or a pharmaceutical composition of claim 44.

46. A method of producing a polypeptide of interest in a cell within a subject, the method comprising administering to the subject the loaded LNP of any one of claims 17-43, or a pharmaceutical composition of claim 44.

47. The method of claim 45 or 46, wherein the cell is an endothelial cell.

48. The method of claim 47, wherein the endothelial cell is a pulmonary endothelial cell, a respiratory endothelial cell, or a bronchial endothelial cell.

49. A method of specifically delivering a therapeutic and/or prophylactic agent to an organ of a subject, the method comprising administering to the subject the loaded LNP of any one of claims 17-43, or a pharmaceutical composition of claim 44.

50. The method of claim 49, wherein the organ is the lung.

51. A method of specifically delivering a therapeutic and/or prophylactic agent to a tissue of a subject, the method comprising administering to the subject the loaded LNP of any one of claims 17-43, or a pharmaceutical composition of claim 44.

52. The method of claim 51, wherein the tissue is the endothelium.

53. The method of claim 51, wherein the tissue is the pulmonary endothelium.

54. A method of treating a disease or disorder in a subject in need thereof, the method comprising administering to the subject a therapeutically effective amount of the loaded LNP of any one claims 17-43, or a pharmaceutical composition of claim 44.

55. The method of any one of claims 45-54, wherein the administering is performed parenterally, intramuscularly, intradermally, subcutaneously, and/or intravenously.
